(19) United States
(12) Patent Application Publication
(10) Pub. No.: US 2024/0208968 A1
(43) Pub. Date: Jun. 27, 2024

(54) ALPHA V BETA 6 AND ALPHA V BETA 1 INTEGRIN INHIBITORS AND USES THEREOF

(71) Applicant: DiCE Molecules SV, Inc., South San Francisco, CA (US)

(72) Inventors: Lalit Kumar SHARMA, South San Francisco, CA (US); John R. JACOBSEN, South San Francisco, CA (US); Paul Ross FATHEREE, South San Francisco, CA (US)

(21) Appl. No.: 18/549,767

(22) PCT Filed: Mar. 10, 2022
(86) PCT No.: PCT/US2022/019759

§ 371 Date: Sep. 8, 2023

Related U.S. Application Data
(60) Provisional application No. 63/159,063, filed on Mar. 10, 2021.

(51) Int. Cl. C07D 471/04; A61K 31/4375; A61K 31/4545; A61K 31/517; A61K 31/519; A61K 31/5377; A61K 31/5386; C07D 519/00

(52) U.S. Cl. CPC C07D 471/04; A61K 31/4375; A61K 31/4545; A61K 31/517; A61K 31/519; A61K 31/5377; A61K 31/5386; C07D 519/00

(57) ABSTRACT

Provided herein are alpha V beta 6 and alpha V beta 1 integrin inhibitors, methods of making such alpha V beta 6 and alpha V beta 1 integrin inhibitors, pharmaceutical compositions of alpha V beta 6 and alpha V beta 1 integrin inhibitors, and methods of treating and/or preventing various medical disorders in a subject by administering to the subject in need thereof alpha V beta 6 and alpha V beta 1 integrin inhibitors.

### CROSS REFERENCE TO RELATED APPLICATIONS

This application is a 371 U.S. National Stage of International Application No. PCT/US2022/019759, filed Mar. 10, 2022, which claims the benefit of U.S. Provisional Application No. 63/159,063, filed on Mar. 10, 2021, each of which are incorporated herein by reference in their entirety.

### FIELD

Provided herein are αVβ6 and αVβ1 integrin inhibitors, methods of making such αVβ6 and αVβ1 integrin inhibitors, pharmaceutical compositions of αVβ6 and αVβ1 integrin inhibitors and methods of treating and/or preventing various medical disorders in a subject by administering to the subject in need thereof αVβ6 and αVβ1 integrin inhibitors.

### BACKGROUND

Integrins are α/β heterodimeric transmembrane proteins involved in cell adhesion to a wide variety of extracellular matrix proteins, which mediate cell-cell interactions, cell migration, cell proliferation, cell survival and maintenance of tissue integrity (Barczyk et al., Cell and Tissue Research 2010, 339, 269). In mammals, there are 24 α/β integrin heterodimers which are derived from combinations of 18 alpha and 8 beta subunits. Transforming Growth Factor β (TGF β) has a central role in driving a number of pathological processes underlying fibrosis, cell growth, and autoimmune diseases. Alpha V (αV) Integrins, that include αVβ1, αVβ3, αVβ5, αVβ6, and αVβ8, are involved in a critical pathway that leads to the conversion of latent TGF β to an active form (Henderson, N. C.; Sheppard, D. Biochim, Biophys. Acta 2013, 1832, 891). Thus, antagonism of such αV integrin mediated activation of latent TGF β provides a viable therapeutic approach to intervene in TGF β driven pathological states (Sheppard, D. Eur. Resp. Rev. 2008, 17, 157; Goodman, S. L.; Picard, M. Trends Pharmacol. Sciences 2012, 33(7), 405; Hinz, B., Nature Medicine 2013, 19(12), 1567; Pozzi, A.; Zent, R. J. Am. Soc. Nephrol. 2013, 24(7), 1034). All five αV integrins belong to a small subset (8 out of 24) of integrins that recognize the Arginine Glycine Aspartic acid (RGD) motif present in native ligands such as fibronectin, vitronectin, and Latency Associated Peptide (LAP).

Integrins are expressed on the surface of most of human cells. For example, αVβ6 and αVol integrins are expressed on epithelial cells at very low levels in healthy tissue but significantly upregulated during inflammation and wound healing. Integrin pathology contributes to a diverse set of human diseases, including, for example, platelet disorders, atherosclerosis, cancer, osteoporosis, fibrosis, diabetic neuropathy of the kidney, macular degeneration and various autoimmune and chronic inflammation diseases.

Accordingly, αVβ6 and αVβ1 integrin inhibitors have been extensively investigated but despite immense effort, therapeutic success has been elusive. Accordingly, there is a need for αVβ6 and αVβ1 integrin inhibitors, which in some embodiments are orally deliverable and may, for example, treat and/or prevent platelet disorders, atherosclerosis, cancer, osteoporosis, fibrosis, diabetic neuropathy of the kidney, macular degeneration and various autoimmune and chronic inflammation diseases.

### BRIEF SUMMARY

In one aspect, provided herein is a compound of Formula (I) which satisfies this and other needs:

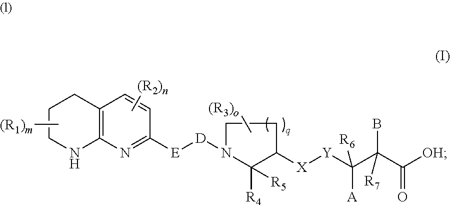

or pharmaceutically acceptable salts, hydrates or solvates thereof wherein:
each R₁ is independently hydrogen, alkyl, substituted alkyl, alkenyl, substituted alkenyl, alkynyl, substituted alkynyl, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl or substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl, substituted heteroarylalkynyl,
halo, —C(O)NR₈R₉, —C(O)OR₁₀, —NR₁₁C(O)OR¹², —NR₁₃C(O)OR¹⁴, —OC(O)OR₁₅, —CN, —CF₃, —NR₁₆S O₂R₁₇ or —OR₁₅; m is 0, 1, 2 or 3; each R₂ is independently hydrogen, alkyl, substituted alkyl, alkenyl, substituted alkenyl, alkynyl, substituted alkynyl, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkanoyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl, substituted heteroarylalkynyl, halo, —C(O)NR₁₉R₂₀, —C(O)OR₂₁, —NR₂₂C(O)OR²³, —NR₂₄C(O)OR₂₅, —OC(O)OR²⁶, —CN, —CF₃, —NR₂₇SO₂R₂₈ or —OR²⁹; n is 0, 1 or 2; each R₃ is independently hydrogen, alkyl, substituted alkyl, alkenyl, substituted alkenyl, alkynyl, substituted alkynyl, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl, substituted heteroarylalkynyl, halo, —C(O)NR₃₀R₃₁, —C(O)OR₃₂, —NR₃₃C(O)OR₃₄, —NR₃₅C(O)OR₃₆, —OC(O)OR₃₇, —CN, —CF₃, —NR₃₈SO₂R₃₉ or —OR⁴⁰; q is 0, 1, 2 or 3; o is 0, 1 or 2 when q is 0; o is 0, 1, 2 or 3 when q is 1; o is 0, 1, 2, 3 or 4 when q is 2; o is 0, 1, 2, 3, 4 or 5 when q is 3; R₄ is hydrogen, alkyl, substituted alkyl, alkenyl, substituted alkenyl, alkynyl, substituted alkynyl, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl, substituted heteroarylalkynyl, —F, —C(O)NR₄₁R₄₂, —C(O)R⁴³, —C(O)OR⁴⁴, —CN, —CF₃ or R₄ and R₅ together with the atom to which they are bonded form a C₄-C₈ cycloalkyl ring;
E is —CH₂— or —CH₂Z—; Z is —NR₄₆—, —S—, —SO₂— or —O—; D is —(CH₂)₂—, —(CH₂)₃—, —CH═CHCH₂—, —C(O)—, —C≡CCH₂—, phenyl, cyclohexyl or cyclopentyl when E is —CH₂—; D is —(CH₂)₂—, —(CH₂)₃—, —C(O)—, phenyl, cyclohexyl or cyclopentyl when Z is NR₄₅ or —O—; D is —(CH₂)₂—, —(CH₂)₃—, phenyl, cyclohexyl or cyclopentyl when Z is —SO₂— or —S—; X—Y is —C(O)NR₄₆—, —NR₄₇C(O)—, —C(O)O—, —CH₂CH₂—, —CH═CH—, —C≡C—, —NR₄₈CH₂—, —CH₂NR₄₉—, —O—CH₂—, —CH₂—O—, —SO₂NR₅₀—, —NR₅₁SO₂— or cyclopropyl; A is hydrogen, —OR⁵², alkyl, substituted alkyl, alkenyl, substituted alkenyl, alkynyl, substituted alkynyl, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl, substituted heteroarylalkynyl or halo; B is hydrogen, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl, substituted heteroarylalkynyl, halo, —NR₅₃R₅₄, —O—R₅₅, —S—R₅₆ or —SO₂-R₅₇; R₈-R₅₃ and R₅₈-R₆₄ are independently hydrogen, alkyl, substituted alkyl, alkenyl, substituted alkenyl, alkynyl, substituted alkynyl, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl or substituted heteroarylalkynyl; R₅₄ is alkyl, substituted alkyl, alkenyl, substituted alkenyl, alkynyl, substituted alkynyl, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl, substituted heteroarylalkynyl, —C(O)R₅₅, —C(O)OR⁵⁹, —C(O)NR₆₀R₆₁, or —SO₂R₆₂;
R₅₅-R₅₇ are alkyl, substituted alkyl, alkenyl, substituted alkenyl, alkynyl, substituted alkynyl, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl or substituted heteroarylalkynyl; R₅ is hydrogen or —F; R₆ is hydrogen, —F or —OR⁶³; and R₇ is hydrogen, —F or —OR₆₄; provided that R₅ is hydrogen when R₄ is —C(O)NR₄₁R₄₂, —C(O)R⁴³, —C(O)OR⁴⁴ or —CN; provided that A is not hydrogen, halo or —OR⁵² when B is hydrogen or halo; provided that B is not hydrogen or halo when A is hydrogen, halo or —OR⁵²; provided that R₇ is hydrogen when B is halo, —NR₅₃R₅₄, —O—R₅₅, —S—R₅₆ or —SO₂R₅₇; provided that R₆ is —OR³ only when X—Y are —CH₂CH₂—, —CH═CH—, —C≡C— or cyclopropyl; provided that A is not —OR₅₂ when R₆ is —OR⁶³; and provided that A is not —Cl, —Br or —I when R₆ is —F.

In another aspect, derivatives, including salts, esters, enol ethers, enol esters, solvates, hydrates, metabolites and prodrugs of the compounds of Formula (I) described herein are provided. Further provided are pharmaceutical compositions which include the compounds of Formula (I) provided herein and a pharmaceutically acceptable vehicle.

Methods of treating, preventing, or ameliorating symptoms of medical disorders such as, for example, platelet disorders, atherosclerosis, cancer, osteoporosis, fibrosis, diabetic neuropathy of the kidney, macular degeneration and various autoimmune and chronic inflammation diseases are provided herein. In practicing the methods, therapeutically effective amounts of the compounds of Formula (I) or pharmaceutical compositions thereof are administered to the patient with the disorder or condition.

Methods for inhibiting αVβ6 integrin in a patient are described herein. In practicing the methods, therapeutically effective amounts of the compounds of Formula (I) or pharmaceutical compositions thereof are administered to the patient.

Methods for inhibiting αVβ1 integrin in a patient are described herein. In practicing the methods, therapeutically effective amounts of the compounds of Formula (I) or pharmaceutical compositions thereof are administered to the patient.

Methods for inhibiting TGFβ activation in a cell are provided herein. In practicing the methods, effective amounts of the compounds or Formula (I) of pharmaceutical compositions thereof are administered to the cell.

In one aspect, provided herein is a compound of Formula (Ia) which satisfies this and other needs:

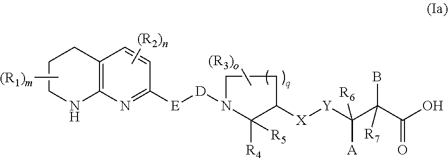

or pharmaceutically acceptable salts thereof, wherein:
q is 1, 2 or 3;
R¹ and R³ are each independently selected at each occurrence from halogen, C₁₋₄ alkyl, C₁₋₄ haloalkyl, —OR¹¹, —SR¹¹, —N(R¹¹)₂, —C(O)N(R¹¹)₂, —C(O)OR¹¹, ═O, ═S, and —CN;
m is selected from 0, 1, 2, 3, 4, 5, and 6;
o is selected from 0, 1, 2, 3, 4, 5, 6, 7, and 8;
R² is independently selected at each occurrence from halogen, C₁₋₄ alkyl, C₁₋₄ haloalkyl, —OR¹², —SR², —N(R¹²)₂, —CN, and —NO₂;
n is 0, 1 or 2;
R⁴ and R⁵ are each independently selected from hydrogen, halogen, C₁₋₄ alkyl, C₁₋₄haloalkyl. —OR¹³, —SR¹³, —N(R¹³)₂, and —CN; or R₄ and R₅ are taken together to form a double bonded substituent selected from ═O, ═S and ═N(R¹³);
D is selected from a bond, —C(O)—, —C≡CCH₂—, and —CH═CHCH₂—;
E is selected from C₁₋₄ alkylene and —(CH₂)Z—,
   wherein Z is selected from —NH—, —S—, —SO₂—, and —O—; X—Y is selected from: ^{λ}—C(O)N(R¹⁴)—, ^{λ}—N(R¹⁴)C(O)—, ^{λ}—N(R⁴)C(O)C(R¹⁴)₂—, ^{λ}—C(O)O—, ^{λ}—C(R¹⁵)₂C(R₁₅)₂—, ^{λ}—CH≡CH—, ^{λ}—C≡C—, ^{λ}—N(R¹⁴)C(R¹⁵)₂—, ^{λ}—C(R¹⁵)₂N(R¹⁴)—, ^{λ}—O—, ^{λ}—C(R¹⁵)₂—, ^{λ}—C(R¹⁵)₂—, ^{λ}—SO₂N(R¹⁴)—, and ^{λ}—N(R¹⁴)SO₂—;
wherein λ denotes the attachment of X—Y to

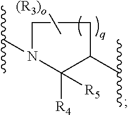

R⁶ and R⁷ are each independently selected at each occurrence from:
   hydrogen, halogen, C₁₋₄ alkyl, C₁₋₄haloalkyl, —OR¹⁶, and —CN:
A is selected from (i) and (ii):
   (i) hydrogen, halogen, and —CN, or A and R⁶ come together to form a C₃₋₆ carbocycle or 3- to 6-membered heterocycle;
   (ii) —OR¹⁷, —SR¹⁷, —N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —C(O)R¹⁷, —C(O)OR¹⁷, —OC(O)R¹⁷, —OC(O)N(R)₂, —C(O)N(R¹⁷)₂, —S(O)R¹⁷, —S(O)₂R¹⁷, and —S(O)₂N(R¹⁷)₂:
      C₁₋₆ alkyl optionally substituted with one or more substituents independently selected from:
         halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═N(R¹⁷), —N₃, and —CN, C₃₋₁₀ carbocycle, and 3- to 10-membered heterocycle,
         wherein the C₃₋₁₀ carbocycle and 3- to 10-membered heterocycle are each optionally substituted with one or more substituents independently selected from halogen, C₁₋₆ alkyl, C₁₋₆ haloalkyl, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═N(R¹⁷), —N₃, and —CN; and
      C₃₋₁₂ carbocycle and 3- to 12-membered heterocycle, any of which is optionally substituted with one or more substituents independently selected from:
         halogen, —OR¹⁷, —SR¹⁷, —N(R¹⁷)₂, —C(O)R¹⁷, —C(O)OR¹⁸, —OC(O)R¹⁸, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═N(R¹⁷), —N₃, and —CN;
         C₁₋₆ alkyl optionally substituted with one or more substituents independently selected from halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═N(R¹⁷), —N₃, and —CN; and
         C₃₋₁₀ carbocycle and 3- to 10-membered heterocycle, any of which is optionally substituted with one or more substituents independently selected from halogen, C₁₋₄ alkyl, C₁₋₄ haloalkyl, and ═O:
B is selected from (I) when A is selected from (ii), or
   B is selected from (11) when A is selected from (i):
   (I) hydrogen, halogen, and —CN, or B and R⁷ are taken together to form a C₃₋₆ carbocycle or a 3- to 6-membered heterocycle;
   (II) —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, and —S(O)₂N(R¹⁸)₂;
      C₁₋₆ alkyl optionally substituted with one or more substituents independently selected from:
         halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═N(R¹⁸), —N₃, —CN, C₃₋₁₀ carbocycle and 3- to 10-membered heterocycle,
         wherein the C₃₋₁₀ carbocycle and 3- to 10-membered heterocycle are optionally substituted with one or more substituents independently selected from halogen, C₁₋₆ alkyl, C₁₋₆ haloalkyl, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═N(R¹⁸), —N₃, and —CN; and
      C₃₋₁₂ carbocycle and 3- to 12-membered heterocycle, any of which is optionally substituted with one or more substituents independently selected from:
         halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═N(R¹⁸), —N₃, and —CN;
         C₁₋₆ alkyl optionally substituted with one or more substituents independently selected from:
         halogen, —OR¹⁸, —SR¹⁸, —N(R¹⁸)₂, —C(O)R¹⁸, —C(O)OR¹⁸. —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═N(R¹⁸), —N₃, —CN, C₃₋₆ carbocycle and 3- to 6-membered heterocycle,
         wherein the C₃₋₆ carbocycle and 3- to 6-membered heterocycle are each optionally substituted with one or more substituents independently selected from halogen, C₁₋₄alkyl, C₁₋₄ haloalkyl, and ═O; and
         C₃₋₁₀ carbocycle and 3- to 10-membered heterocycle, any of which is optionally substituted with one or more substituents independently selected from halogen, C₁₋₄ alkyl, C₁₋₄haloalkyl, and ═O;
R¹¹, R¹², R¹³, R¹⁴, and R¹⁵ are each independently selected at each occurrence from hydrogen, C₁₋₄ alkyl, and C₁₋₄haloalkyl;
R¹⁵ is independently selected at each occurrence from hydrogen, halogen, C₁₋₄ alkyl, and C₁₋₄ haloalkyl;
R¹⁷ is independently selected at each occurrence from:
   hydrogen;
   C₁₋₆ alkyl optionally substituted with one or more substituents independently selected from: halogen, —OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —NO₂, ═O, ═S, ═N(R²¹), —N₃, and —CN; and
   C₃₋₆ carbocycle and 3- to 6-membered heterocycle, any of which are optionally substituted with one or more substituents independently selected from: halogen, C₁₋₄ alkyl, C₁₋₄ haloalkyl, —OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —N(R²¹)C(O)OR²¹, —N(R²¹)C(O)N(R²¹)₂, —N(R²¹)C(S)N(R²¹)₂, —N(R²¹)S(O)₂(R²¹), —S(O)R²¹, —S(O)₂R²¹, —S(O)₂N(R²¹)₂, —NO₂, ═O, ═S, ═N(R²¹), —N₃, and —CN;
R¹⁸ is independently selected at each occurrence from:
   hydrogen;
   C₁₋₆ alkyl optionally substituted with one or more substituents independently selected from: halogen, —OR²², —SR²², —N(R²²)₂, —C(O)R²², —C(O)OR²², —OC(O)R²², —OC(O)N(R²)₂, —C(O)N(R²²)₂, —N(R²²)C(O)R²², —NO₂, ═O, ═S, ═N(R²²), —N₃, —CN, C₃₋₁₀ carbocycle, and 3- to 10-membered heterocycle,
      wherein the C₃₋₁₀ carbocycle and 3- to 10-membered heterocycle each are optionally substituted with one or more substituents independently selected from halogen, C₁₋₆ alkyl, C₁₋₆ haloalkyl, —OR²², —SR²², and —N(R²²)₂; and
   C₃₋₁₀ carbocycle and 3- to 10-membered heterocycle, any of which is optionally substituted with one or more substituents independently selected from:
      halogen, C₁₋₆ alkyl, C₁₋₆ haloalkyl, —OR²², —SR²², —N(R²²)₂, —C(O)R²², —C(O)OR²², —OC(O)R²², —OC(O)N(R²²)₂, —C(O)N(R²²)₂, —N(R²²)C(O)R²². —N(R²²)C(O)OR²², —N(R²²)C(O)N(R²²)₂, —N(R²²)C(S)N(R²²)₂, —N(R²²)S(O)₂(R²²), —S(O)R²², —S(O)₂R²², —S(O)₂N(R²²)₂, —NO₂, ═O, ═S, ═N(R²²), —N₃, —CN, C₃₋₆ carbocycle, and 3- to 6-membered heterocycle;
         wherein the C₃₋₆ carbocycle and 3- to 6-membered heterocycle are each optionally substituted with one or more substituents independently selected from halogen, C₁₋₄ alkyl, and C₁₋₄ haloalkyl:
R²¹ and R²² are each independently selected at each occurrence from:
   hydrogen;
   C₁₋₄ alkyl optionally substituted with one or more substituents independently selected from halogen, hydroxyl, C₃₋₄ carbocycle, and 3- to 6-membered heterocycle, wherein each C₃₋₆ carbocycle and 3- to 6-membered heterocycle is optionally substituted with one or more substituents independently selected from C₁₋₄ alkyl, —N(R²³)₂, and —C(O)N(R²³)₂; and
   C₃₋₆ carbocycle and 3- to 12-membered heterocycle, any of which is optionally substituted with one or more substituents independently selected from halogen, C₁₋₄ alkyl, C₁₋₄ haloalkyl, C₁₋₄ alkoxy, and ═O; and
R²³ is independently selected at each occurrence from hydrogen and C₁₋₄alkyl.

In one aspect, the present disclosure provides a pharmaceutical composition comprising pharmaceutically acceptable excipient and a compound or salt of Formula (Ia).

In one aspect, the present disclosure provides a method of modulating an alpha V integrin in a subject in need thereof, comprising administering to the subject a compound or salt of Formula (Ia) or a pharmaceutical composition of Formula (Ia).

In some embodiments, the alpha V integrin is an alpha V beta 1 integrin.

In some embodiments, the alpha V integrin is an alpha V beta 6 integrin.

In one aspect, the present disclosure provides a method of treating a disease or condition comprising administering to a subject in need thereof a compound or salt of Formula (Ia) or a pharmaceutical composition comprising a compound or salt of Formula (Ia).

In some embodiments, the disease or condition is selected from: idiopathic pulmonary fibrosis, systemic lupus erythematosus associated interstitial lung disease, rheumatoid arthritis, diabetic nephropathy, focal segmental glomerulosclerosis, chronic kidney disease, nonalcoholic steatohepatitis, primary biliary cholangitis, primary sclerosing cholangitis, solid tumors, hematological tumors, organ transplant, Alport syndrome, interstitial lung disease, radiation-induced fibrosis, bleomycin-induced fibrosis, asbestos-induced fibrosis, flu-induced fibrosis, coagulation-induced fibrosis, vascular injury-induced fibrosis, aortic stenosis, and cardiac fibrosis.

### BRIEF DESCRIPTION OF THE DRAWINGS

FIG. 1 illustrates Scheme 1 which describes the synthesis of intermediate 10.

FIG. 2 illustrates Scheme 2 which describes a synthesis of compounds of Formula (VII).

FIG. 3 illustrates Scheme 4 which describes another synthesis of compounds of Formula (VII).

FIG. 4 illustrates Scheme 6 which describes synthesis of compounds of Formula (VIII).

FIG. 5 illustrates Scheme 10 which describes another synthesis of compounds of Formula (VIII).

FIG. 6 illustrates Scheme 12 which describes the synthesis of amides and sulfonamides of Formula (VIII).

FIG. 7 illustrates Scheme 14 which describes the synthesis of compounds where the central piperdine ring is substituted and/or E-D are not propyl.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

### Definitions

Unless defined otherwise, all technical and scientific terms used herein have the same meaning as is commonly understood by one of ordinary skill in the art to which this invention belongs. If a plurality of definitions for a term exist herein, those in this section prevail unless stated otherwise.

As used herein, and unless otherwise specified, the terms “about” and “approximately,” when used in connection with a property with a numeric value or range of values indicate that the value or range of values may deviate to an extent deemed reasonable to one of ordinary skill in the art while still describing the particular property. Specifically, the terms “about” and “approximately,” when used in this context, indicate that the numeric value or range of values may vary by 5%, 4%, 3% 2%, 1%, 0.9%, 0.8%, 0.7%, 0.6%, 0.5%, 0.4%, 0.3%, 0.2% or 0.1% of the recited value or range of values.

“Alkyl,” by itself or as part of another substituent, refers to a saturated, branched or straight-chain monovalent hydrocarbon radical derived by the removal of one hydrogen atom from a single carbon atom of a parent alkane. Typical alkyl groups include, but are not limited to, methyl; ethyls; propyls such as propan-1-yl, propan-2-yl, etc.; butyls such as butan-1-yl, butan-2-yl, 2-methyl-propan-1-yl, 2-methyl-propan-2-yl, etc.; and the like. In some embodiments, an alkyl group comprises from 1 to 20 carbon atoms (C₁-C₂₀ alkyl). In other embodiments, an alkyl group comprises from 1 to 10 carbon atoms (C₁-C₁₀ alkyl). In still other embodiments, an alkyl group comprises from 1 to 6 carbon atoms (C₁-C₆ alkyl).

“Alkenyl,” by itself or as part of another substituent, refers to a branched or straight-chain alkyl radical having at least one carbon-carbon double bond. The radical is derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like. In some embodiments, an alkenyl group comprises from 1 to 20 carbon atoms (C₁-C₂₀ alkenyl). In other embodiments, an alkenyl group comprises from 1 to 10 carbon atoms (C₁-C₁₀ alkenyl). In still other embodiments, an alkenyl group comprises from 1 to 6 carbon atoms (C₁-C₆ alkenyl).

“Alkynyl,” by itself or as part of another substituent refers to a branched or straight-chain alkyl radical having at least one carbon-carbon triple bond. The radical is derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like. In some embodiments, an alkynyl group comprises from 1 to 20 carbon atoms (C₁-C₂₀ alkynyl). In other embodiments, an alkynyl group comprises from 1 to 10 carbon atoms (C₁-C₁₀ alkynyl). In still other embodiments, an alkynyl group comprises from 1 to 6 carbon atoms (C₁-C₆ alkynyl).

“Aryl,” by itself or as part of another substituent, refers to a monovalent aromatic hydrocarbon group derived by the removal of one hydrogen atom from a single carbon atom of a parent aromatic ring system, as defined herein. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In some embodiments, an aryl group comprises from 6 to 20 carbon atoms (C₆-C₂₀ aryl). In other embodiments, an aryl group comprises from 6 to 15 carbon atoms (C₆-C₁₅ aryl). In still other embodiments, an aryl group comprises from 6 to 10 carbon atoms (C₆-C₁₀ aryl).

“Arylalkyl.” by itself or as part of another substituent, refers to an acyclic alkyl group in which one of the hydrogen atoms bonded to a carbon atom, typically a terminal or sp³ carbon atom, is replaced with an aryl group as, as defined herein. Typical arylalkyl groups include, but are not limited to, benzyl. 2-phenylethan-1-yl. 2-phenylethen-1-yl, naphthylmethyl, 2-naphthylethan-1-yl, 2-naphthylethen-1-yl, naphthobenzyl, 2-naphthophenylethan-1-yl and the like. In some embodiments, an arylalkyl group is (C₆-C₃₀) arylalkyl, e.g., the alkyl moiety of the arylalkyl group is (C₁-C₁₀) alkyl and the aryl moiety is (C₆-C₂₀) aryl. In other embodiments, an arylalkyl group is (C₆-C₂₀) arylalkyl, e.g., the alkyl moiety of the arylalkyl group is (C₁-C₅) alkyl and the aryl moiety is (C₆-C₁₂) aryl. In still other embodiments, an arylalkyl group is (C₆-C₁₅) arylalkyl, e.g., the alkyl moiety of the arylalkyl group is (C₁-C₅) alkyl and the aryl moiety is (C₆-C₁₀) aryl.

“Arylalkenyl.” by itself or as part of another substituent, refers to an acyclic alkenyl group in which one of the hydrogen atoms bonded to a carbon atom, is replaced with an aryl group as, as defined herein. In some embodiments, an arylalkenyl group is (C₆-C₃₀) arylalkenyl, e.g., the alkenyl moiety of the arylalkenyl group is (C₁-C₁₀) alkenyl and the aryl moiety is (C₁-C₂₀) aryl. In other embodiments, an arylalkenyl group is (C₆-C₂₀) arylalkenyl, e.g., the alkenyl moiety of the arylalkenyl group is (C₁-C₅) alkenyl and the aryl moiety is (C₆-C₂) aryl. In still other embodiments, an arylalkenyl group is (C₆-C₁₅) arylalkenyl, e.g., the alkenyl moiety of the arylalkenyl group is (C₁-C₅) alkenyl and the aryl moiety is (C₆-C₁₀) aryl.

“Arylalkynyl,” by itself or as part of another substituent, refers to an acyclic alkynyl group in which one of the hydrogen atoms bonded to a carbon atom, is replaced with an aryl group as, as defined herein. In some embodiments, an arylalkynyl group is (C₆-C₃₀) arylalkynyl, e.g., the alkynyl moiety of the arylalkynyl group is (C₁-C₁₀) alkynyl and the aryl moiety is (C₆-C₂₀) aryl. In other embodiments, an arylalkynyl group is (C₆-C₂₀) arylalkynyl, e.g., the alkynyl moiety of the arylalkynyl group is (C₁-C₅) alkynyl and the aryl moiety is (C₆-C₁₂) aryl. In still other embodiments, an arylalkynyl group is (C₆-C₁₅) arylalkynyl, e.g., the alkynyl moiety of the arylalkynyl group is (C₁-C₅) alkynyl and the aryl moiety is (C₆-C₁₀) aryl.

“Carbocycle” as used herein refers to a saturated, unsaturated or aromatic ring in which each atom of the ring is carbon. Carbocycle include 3- to 10-membered monocyclic rings and 6- to 12-membered bicyclic rings. Each ring of a bicyclic carbocycle may be selected from saturated, unsaturated, and aromatic rings. Bicyclic carbocycles may be fused, bridged or spiro-ring systems.

In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl.

Carbocycle may be optionally substituted by one or more substituents such as those substituents described herein.

“Cycloalkyl,” by itself or as part of another substituent, refers to a saturated cyclic monovalent hydrocarbon radical derived by the removal of one hydrogen atom from a single carbon atom of a parent cycloalkane. Typical cycloalkyl groups include, but are not limited to, cyclopropyl, cyclobutyl, cyclopentyl cycopentenyl; etc.; and the like. In some embodiments, a cycloalkyl group comprises from 3 to 15 carbon atoms (C₃-C₁₅ cycloalkyl). In other embodiments, a cycloalkyl group comprises from 3 to 10 carbon atoms (C₃-C₁₀ cycloalkyl). In still other embodiments, a cycloalkyl group comprises from 3 to 8 carbon atoms (C₃-C₈ cycloalkyl). The term “cycloalkyl” also includes multicyclic hydrocarbon ring systems having a single radical and between 5 and 15 carbon atoms. Exemplary multicyclic cycloalkyl rings include bridged, fused, and spiro cycloalkyl ring systems, including, for example, norbornyl, pinyl, and adamantyl.

“Cycloalkenyl,” by itself or as part of another substituent, refers to an unsaturated cyclic monovalent hydrocarbon radical derived by the removal of one hydrogen atom from a single carbon atom of a parent cycloalkene. Typical cycloalkenyl groups include, but are not limited to, cyclopropene, cyclobutene cyclopentene; etc.; and the like. In some embodiments, a cycloalkenyl group comprises from 3 to 15 carbon atoms (C₃-C₁₅ cycloalkenyl). In other embodiments, a cycloalkenyl group comprises from 3 to 10 carbon atoms (C₃-C₁₀ cycloalkenyl). In still other embodiments, a cycloalkenyl group comprises from 3 to 8 carbon atoms (C₃-C₈ cycloalkenyl). The term “cycloalkenyl” also includes multicyclic hydrocarbon ring systems having a single radical and between 5 and 15 carbon atoms with an alkenyl group.

“Cycloheteroalkyl” by itself or as part of another substituent, refers to a cycloalkyl group as defined herein in which one or more one or more of the carbon atoms (and optionally any associated hydrogen atoms), are each, independently of one another, replaced with the same or different heteroatoms or heteroatomic groups as defined in “heteroalkyl” below. In some embodiments, a cycloheteroalkyl group comprises from 3 to 15 carbon atoms (C₃-C₁₅ cycloheteroalkyl). In other embodiments, a cycloheteroalkyl group comprises from 3 to 10 carbon atoms (C₃-C₁₀ cycloheteroalkyl). In still other embodiments, a cycloheteroalkyl group comprises from 3 to 8 carbon atoms (C₃-C₈ cycloheteroalkyl). The term “cycloheteroalkyl” also includes multicyclic hydrocarbon ring systems with at least one heteroatom having a single radical and between 5 and 15 carbon atoms.

“Cycloheteroalkenyl,” by itself or as part of another substituent, refers to a cycloalkenyl group as defined herein in which one or more one or more of the carbon atoms (and optionally any associated hydrogen atoms), are each, independently of one another, replaced with the same or different heteroatoms or heteroatomic groups as defined in “heteroalkenyl” below. In some embodiments, a cycloheteroalkenyl group comprises from 3 to 15 carbon atoms (C₃-Cis cycloheteroalkenyl). In other embodiments, a cycloheteroalkyl group comprises from 3 to 10 carbon atoms (C₃-C₁₀ cycloheteroalkenyl). In still other embodiments, a cycloheteroalkyl group comprises from 3 to 8 carbon atoms (C₃-C₈ cycloheteroalkenyl). The term “cycloheteroalkenyl” also includes multicyclic hydrocarbon ring systems with at least one heteroatom and one alkenyl group having a single radical and between 5 and 15 carbon atoms.

“Compounds,” refers to compounds encompassed by structural formulae disclosed herein and includes any specific compounds within these formulae whose structure is disclosed herein.

Compounds may be identified either by their chemical structure and/or chemical name. The compounds described herein may contain one or more chiral centers and/or double bonds and therefore, may exist as stereoisomers, such as double-bond isomers (i.e., geometric isomers), enantiomers or diastereomers. Accordingly, the chemical structures depicted herein encompass the stereoisomerically pure form depicted in the structure (e.g., geometrically pure, enantiomerically pure or diastereomerically pure). The chemical structures depicted herein also encompass the enantiomeric and stereoisomeric derivatives of the compound depicted. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁸O, ¹⁷O, etc.

Compounds may exist in unsolvated forms as well as solvated forms, including hydrated forms. In general, compounds may be hydrated or solvated. Certain compounds may exist in multiple crystalline or amorphous forms. In general, all physical forms are equivalent for the uses contemplated herein and are intended to be within the scope of the present disclosure. Further, it should be understood, when partial structures of the compounds are illustrated, that wavy lines indicate the point of attachment of the partial structure to the rest of the molecule.

“Halo,” by itself or as part of another substituent refers to a radical —F, —Cl, —Br or —I.

“Heteroalkyl,” by itself or as part of another substituent, refer to an alkyl group, in which one or more of the carbon atoms (and optionally any associated hydrogen atoms), are each, independently of one another, replaced with the same or different heteroatoms or heteroatomic groups. Typical heteroatoms or heteroatomic groups which can replace the carbon atoms include, but are not limited to, —O—, —S—, —N—, —Si—, —NH—, —S(O)—, —S(O)₂—, —S(O)NH—, —S(O)₂NH— and the like and combinations thereof. The heteroatoms or heteroatomic groups may be placed at any interior position of the alkyl group. Typical heteroatomic groups which can be included in these groups include, but are not limited to, —O—, —S—, —O—O—, —S—S—, —O—S—, —NR⁵⁰¹R⁵⁰², ═N—N═, —N═N—, —N═N—NR⁵⁰³R⁵⁰⁴, —PR⁵⁰⁵—, —P(O)₂—, —POR⁵⁰⁶—, —O—P(O)₂—, —SO—, —SO₂—, —SnR⁵⁰⁷R⁵⁰⁸ and the like, where R⁵⁰¹, R⁵⁰², R⁵⁰³, R⁵⁰⁴, R⁵⁰⁵, R⁵⁰⁶, R⁵⁰⁷ and R⁵⁰⁸ are independently hydrogen, alkyl, alkenyl, alkynyl, aryl, arylalkyl, arylalkenyl, arylalkynyl, heteroalkyl, heteroalkenyl, heteroalkynyl, heteroaryl, heteroarylalkyl, heteroarylalkenyl or heteroarylalkynyl and their substituted counterparts.

“Heteroalkenyl,” refers to an alkenyl group in which one or more of the carbon atoms (and optionally any associated hydrogen atoms), are each, independently of one another, replaced with the same or different heteroatoms or heteroatomic groups. Typical heteroatoms or heteroatomic groups which can replace the carbon atoms include, but are not limited to, —O—, —S—, —N—, —Si—, —NH—, —S(O)—, —S(O)₂—, —S(O)NH—, —S(O)₂NH— and the like and combinations thereof. The heteroatoms or heteroatomic groups may be placed at any interior position of the alkenyl group. Typical heteroatomic groups which can be included in these groups include, but are not limited to, —O—, —S—, —O—O—, —S—S—, —O—S—, —NR⁵⁰⁹R⁵¹⁰, ═N—N═, —N═N—, —N═N—NR⁵¹¹R⁵¹², —PR⁵¹⁴—, —P(O)₂—, —POR⁵¹⁴—, —O—P(O)₂—, —SO—, —SO₂—, —SnR⁵¹⁵R⁵¹⁶ and the like, where R⁵⁰⁹, R⁵¹⁰, R⁵¹¹, R⁵¹², R⁵¹³, R⁵¹⁴, R⁵¹⁵ and R⁵¹⁶ are independently hydrogen, alkyl, aryl, substituted aryl, heteroalkyl, heteroaryl or substituted heteroaryl.

“Heteroalkynyl,” by itself or as part of another substituent, refers to an alkynyl group in which one or more of the carbon atoms (and optionally any associated hydrogen atoms), are each, independently of one another, replaced with the same or different heteroatoms or heteroatomic groups. Typical heteroatoms or heteroatomic groups which can replace the carbon atoms include, but are not limited to, —O—, —S—, —N—, —Si—, —NH—, —S(O)—, —S(O)₂—, —S(O)NH—, —S(O)₂NH— and the like and combinations thereof. The heteroatoms or heteroatomic groups may be placed at any interior position of the alkynyl group. Typical heteroatomic groups which can be included in these groups include, but are not limited to, —O—, —S—, —O—O—, —S—S—, —O—S—, —NR⁵¹⁷R⁵¹⁸, ═N—N═, —N═N—, —N═N—NR⁵¹⁹R⁵²¹, —PR⁵²¹—, —P(O)₂—, —POR⁵²²—, —O—P(O)₂—, —SO—, —SO₂—, —SnR⁵²³R⁵²⁴ and the like, where R⁵¹⁷, R⁵¹⁸, R⁵¹⁹, R⁵²⁰, R⁵²¹, R⁵²², R⁵²³ and R⁵²⁴ are independently hydrogen, alkyl, aryl, substituted aryl, heteroalkyl, heteroaryl or substituted heteroaryl.

“Heteroaryl,” by itself or as part of another substituent, refers to a monovalent heteroaromatic radical derived by the removal of one hydrogen atom from a single atom of a parent heteroaromatic ring systems, as defined herein. Typical heteroaryl groups include, but are not limited to, groups derived from acridine, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In some embodiments, the heteroaryl group comprises from 5 to 20 ring atoms (5-20 membered heteroaryl). In other embodiments, the heteroaryl group comprises from 5 to 10 ring atoms (5-10 membered heteroaryl). Exemplary heteroaryl groups include those derived from furan, thiophene, pyrrole, benzothiophene, benzofuran, benzimidazole, indole, pyridine, pyrazole, quinoline, imidazole, oxazole, isoxazole and pyrazine.

“Heteroarylalkyl,” by itself or as part of another substituent refers to an acyclic alkyl group in which one of the hydrogen atoms bonded to a carbon atom, typically a terminal or sp³ carbon atom, is replaced with a heteroaryl group. In some embodiments, the heteroarylalkyl group is a 6-21 membered heteroarylalkyl, e.g., the alkyl moiety of the heteroarylalkyl is (C₁-C₆) alkyl and the heteroaryl moiety is a 5-15-membered heteroaryl. In other embodiments, the heteroarylalkyl is a 6-13 membered heteroarylalkyl, e.g., the heteroalkyl moiety is (C₁-C₃) alkyl and the heteroaryl moiety is a 5-10 membered heteroaryl.

“Heteroarylalkenyl,” by itself or as part of another substituent refers to an acyclic alkenyl group in which one of the hydrogen atoms bonded to a carbon atom, is replaced with a heteroaryl group. In some embodiments, the heteroarylalkenyl group is a 5-21 membered heteroarylalkenyl, e.g., the alkenyl moiety of the heteroarylalkenyl is (C₂-C₆) alkenyl and the heteroaryl moiety is a 3-15-membered heteroaryl. In other embodiments, the heteroarylalkenyl is a 6-13 membered heteroarylalkenyl, e.g., the alkenyl moiety is (C₃) alkenyl and the heteroaryl moiety is a 3-10 membered heteroaryl.

“Heteroarylalkynyl,” by itself or as part of another substituent refers to an acyclic alkynyl group in which one of the hydrogen atoms bonded to a carbon atom, is replaced with a heteroaryl group. In some embodiments, the heteroarylalkynyl group is a 5-21 membered heteroarylalkynyl, e.g., the alkynyl moiety of the heteroarylalkynyl is (C₂-C₆) alkynyl and the heteroaryl moiety is a 3-15-membered heteroaryl. In other embodiments, the heteroarylalkynyl is a 6-13 membered heteroarylalkynyl, e.g., the alkynyl moiety is (C₃) alkynyl and the heteroaryl moiety is a 3-10 membered heteroaryl.

“Heterocycle” as used herein refers to a saturated, unsaturated, non-aromatic or aromatic ring comprising one or more heteroatoms. Exemplary heteroatoms include N, O, Si, P, B, and S atoms. Heterocycles include 3- to 10-membered monocyclic rings and 6- to 12-membered bicyclic rings. Each ring of a bicyclic heterocycle may be selected from saturated, unsaturated, and aromatic rings. In some embodiments, the heterocycle comprises at least one heteroatom selected from oxygen, nitrogen, sulfur, or any combination thereof. In some embodiments, the heterocycle comprises at least one heteroatom selected from oxygen, nitrogen, or any combination thereof. In some embodiments, the heterocycle comprises at least one heteroatom selected from oxygen, sulfur, or any combination thereof. In some embodiments, the heterocycle comprises at least one heteroatom selected from nitrogen, sulfur, or any combination thereof. The heterocycle may be attached to the rest of the molecule through any atom of the heterocycle, valence permitting, such as a carbon or nitrogen atom of the heterocycle. In some embodiments, the heterocycle is a heteroaryl. In some embodiments, the heterocycle is a heterocycloalkyl. Exemplary heterocycles include pyrrolidinyl, pyrrolyl, imidazolyl, pyrazolyl, triazolyl, piperidinyl, pyridinyl, pyrimidinyl, pyridazinyl, pyrazinyl, thiophenyl, oxazolyl, thiazolyl, morpholinyl, indazolyl, indolyl, and quinolinyl. Bicyclic heterocycles may be fused, bridged or spiro-ring systems. In an exemplary embodiment, a heterocycle, e.g., pyridyl, may be fused to a saturated or unsaturated ring, e.g., cyclohexane, cyclopentane, or cyclohexene. Heterocycle may be optionally substituted by one or more substituents such as those substituents described herein.

“Hydrates,” refers to incorporation of water into to the crystal lattice of a compound described herein, in stoichiometric proportions, resulting in the formation of an adduct. The hydrated forms of the compounds presented herein are also considered to be disclosed herein. Methods of making hydrates include, but are not limited to, storage in an atmosphere containing water vapor, dosage forms that include water, or routine pharmaceutical processing steps such as, for example, crystallization (i.e., from water or mixed aqueous solvents), lyophilization, wet granulation, aqueous film coating, or spray drying. Hydrates may also be formed, under certain circumstances, from crystalline solvates upon exposure to water vapor, or upon suspension of the anhydrous material in water. Hydrates may also crystallize in more than one form resulting in hydrate polymorphism. See e.g., (Guillory, K., Chapter 5, pp. 202205 in Polmorphism in Pharmaceutical Solids, (Brittain, H, ed.), Marcel Dekker. Inc., New York, NY, 1999). The above methods for preparing hydrates are well within the ambit of those of skill in the art, are completely conventional and do not require any experimentation beyond what is typical in the art. Hydrates may be characterized and/or analyzed by methods well known to those of skill in the art such as, for example, single crystal X-ray diffraction, X-ray powder diffraction, polarizing optical microscopy, thermal microscopy, thermogravimetry, differential thermal analysis, differential scanning calorimetry. IR spectroscopy, Raman spectroscopy and NMR spectroscopy. (Brittain, H., Chapter 6, pp. 205-208 in Polymorphism in Pharmaceutical Solids, (Brittain, H, ed.), Marcel Dekker. Inc. New York, 1999). In addition, many commercial companies routine offer services that include preparation and/or characterization of hydrates such as, for example, HOLODIAG, Pharmaparc II, Voie de l′Innovation, 27 100 Val de Reuil, France (http.//www.holodiag.com).

“Parent Aromatic Ring System,” refers to an unsaturated cyclic or polycyclic ring system having a conjugated n electron system. Specifically included within the definition of “parent aromatic ring system” are fused ring systems in which one or more of the rings are aromatic and one or more of the rings are saturated or unsaturated, such as, for example, fluorene, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. The saturated ring system may include one or more heteroatoms.

“Parent Heteroaromatic Ring System.” refers to a parent aromatic ring system in which one or more carbon atoms (and optionally any associated hydrogen atoms) are each independently replaced with the same or different heteroatom. Typical heteroatoms to replace the carbon atoms include, but are not limited to, N, P, O, S, Si, etc. Specifically included within the definition of “parent heteroaromatic ring system” are fused ring systems in which one or more of the rings are aromatic and one or more of the rings are saturated or unsaturated, such as, for example, benzodioxan, benzofuran, chromane, chromene, indole, indoline, xanthene, etc. Typical parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like. The saturated ring system may include one or more heteroatoms.

“Pharmaceutically acceptable salt,” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) base addition salts, formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Preventing,” or “prevention,” refers to a reduction in risk of acquiring a disease or disorder (i.e., causing at least one of the clinical symptoms of the disease not to develop in a patient that may be exposed to or predisposed to the disease but does not yet experience or display symptoms of the disease). The application of a therapeutic for preventing or prevention of a disease or disorder is known as ‘prophylaxis.’ In some embodiments, the compounds provided herein provide superior prophylaxis because of lower long term side effects over long time periods.

“Protecting group,” refers to a grouping of atoms that when attached to a reactive functional group in a molecule masks, reduces or prevents reactivity of the functional group during chemical synthesis. Examples of protecting groups can be found in Green et al., “Protective Groups in Organic Chemistry”, (Wiley, 2^{nd} ed. 1991) and Harrison et al., “Compendium of Synthetic Organic Methods”, Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

“Solvates,” refers to incorporation of solvents into to the crystal lattice of a compound described herein, in stoichiometric proportions, resulting in the formation of an adduct. In addition, the compounds described herein can exist in unsolvated as well as solvated forms with pharmaceutically acceptable solvents such as water, ethanol, and the like. The solvated forms of the compounds presented herein are also considered to be disclosed herein Methods of making solvates include, but are not limited to, storage in an atmosphere containing a solvent, dosage forms that include the solvent, or routine pharmaceutical processing steps such as, for example, crystallization (i.e., from solvent or mixed solvents) vapor diffusion, etc. Solvates may also be formed, under certain circumstances, from other crystalline solvates or hydrates upon exposure to the solvent or upon suspension material in solvent. Solvates may crystallize in more than one form resulting in solvate polymorphism. See e.g., (Guillory, K., Chapter 5, pp. 205-208 in Polymorphism in Pharmaceutical Solids, (Brittain, H, ed.), Marcel Dekker. Inc., New York, NY, 1999)). The above methods for preparing solvates are well within the ambit of those of skill in the art, are completely conventional do not require any experimentation beyond what is typical in the art. Solvates may be characterized and/or analyzed by methods well known to those of skill in the art such as, for example, single crystal X-ray diffraction, X-ray powder diffraction, polarizing optical microscopy, thermal microscopy, thermogravimetry, differential thermal analysis, differential scanning calorimetry, IR spectroscopy, Raman spectroscopy and NMR spectroscopy. (Brittain, H., Chapter 6, pp. 205208 in Polymorphism in Pharmaceutical Solids, (Brittain, H, ed.), Marcel Dekker. Inc. New York, 1999). In addition, many commercial companies routine offer services that include preparation and/or characterization of solvates such as, for example, HOLODIAG, Pharmaparc II, Voie de l′Innovation, 27 100 Val de Reuil, France (http://www.holodiag.com).

“Substituted,” when used to modify a specified group or radical, means that one or more hydrogen atoms of the specified group or radical are each, independently of one another, replaced with the same or different substituent(s). Substituent groups useful for substituting saturated carbon atoms in the specified group or radical include R^{a}, halo, —O⁻, ═O, —OR^{b}, —SR^{b}, —S⁻, ═S, —NR^{c}R^{c}, ═NR^{b}, ═N—OR^{b}, trihalomethyl, —CF₃, —CN, —OCN, —SCN, —NO, —NO₂, —N—OR^{b}, —N—NR^{c}R^{c}, —NR^{b}S(O)₂R^{b}, ═N₂, —N₃, —S(O)₂R^{b}, —S(O)₂NR^{b}R^{b}, —S(O)₂O⁻, —S(O)₂OR^{b}, —OS(O)₂R^{b}, —OS(O)₂O⁻, —OS(O)₂OR^{b}, —OS(O)₂NR^{c}NR^{c}, —P(O)(O⁻)₂, —P(O)(OR^{b})(O⁻), —P(O)(OR^{b})(OR^{b}), —C(O)R^{b}, —C(O)NR^{b}—OR^{b}—C(S)R^{b}, —C(NR^{b})R^{b}, —C(O)O—, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(O)NR^{c}R^{c}, —OC(NCN)NR^{c}R^{c}—OC(S)OR^{b}, —NR^{b}C(O)R^{b}, —NR^{b}C(S)R^{b}, —NR^{b} C(O)O⁻, —NR^{b}C(O)OR^{b}, —NR^{b}C(NCN)OR^{b}, —NR^{b}S(O)₂NR^{c}R^{c}, —NR^{b}C(S)OR^{b}, —NR^{b}C(O)NR^{c}R^{c}, —NR^{b}C(S)NR^{c}R^{c}, —NR^{b}C(S)NR^{b}C(O)R^{a}, —NR^{b}S(O)₂OR^{b}, —NR^{b}S(O)₂R^{b}, —NR^{b}C(NCN)NR^{c}R^{c}, —NR^{b}C(NR^{b})R^{b} and —NR^{b}C(NR^{b})NR^{c}R^{c}, where each R^{a} is independently, aryl, substituted aryl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl or substituted heteroarylalkynyl; each R^{b} is independently hydrogen, alkyl, substituted alkyl, alkenyl, substituted alkenyl, alkynyl, substituted alkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, arylalkyl, substituted arylalkyl, arylalkenyl, substituted arylalkenyl, arylalkynyl, substituted arylalkynyl, heteroarylalkyl, substituted heteroarylalkyl, heteroarylalkenyl, substituted heteroarylalkenyl, heteroarylalkynyl or substituted heteroarylalkynyl; and each R^{c} is independently R^{b} or alternatively, the two R^{c}s taken together with the nitrogen atom to which they are bonded form a 4-, 5-, 6- or 7 membered-cycloheteroalkyl, substituted cycloheteroalkyl, cycloheteroalkenyl, substituted cycloheteroalkenyl or a cycloheteroalkyl or cycloheteroalkenyl fused with an aryl group which may optionally include from 1 to 4 of the same or different additional heteroatoms selected from the group consisting of O, N and S. As specific examples, —NR^{c}R^{c} is meant to include —NH₂, —NH-alkyl, N-alkenyl, N-pyrrolidinyl and N-morpholinyl. In other embodiments, substituent groups useful for substituting saturated carbon atoms in the specified group or radical include R^{a}, halo, —OR^{b}, —NR^{c}R^{c}, trihalomethyl, ═N—OR^{b}, —CN, —NR^{b}S(O)₂R^{b}, —C(O)R^{b}, —C(O)OR^{b}, —C(O)NR^{c}R^{c}, —OC(O)R^{b}, —OC(O)OR^{b}, —S(O)₂R^{b}, —S(O)₂NR^{c}NR^{c}, —OC(O)NR^{c}R^{c}, and —NR^{b}C(O)OR^{b}, where R^{a}, R^{b} and R^{c} are as previously defined above.

Substituent groups useful for substituting unsaturated carbon atoms in the specified group or radical include —R^{a}, halo, —O⁻, —OR^{b}, —SR^{b}, —S⁻, —NR^{c}R^{c}, trihalomethyl, —CF₃, —CN, —OCN, —SCN, —NO, —NO₂, —N₃, —S(O)₂O—, —S(O)₂OR^{b}, —OS(O)₂R^{b}, —OS(O) ₂OR^{b}, —OS(O)₂O, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O—, —OC(O)OR^{b}, —OC(S)OR^{b}, —OC(O)NR^{c}R^{c}, —OS(O)₂NR^{c}NR^{c}, —NR^{b}C(O)R^{b}, —NR^{b}C(S)R^{b}, —NR^{b}C(O)O⁻, —NR^{b}C(O)OR^{b}, —NR^{b}S(O)₂OR^{a}, —NR^{b}S(O)₂R^{a}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R are as previously defined. In other embodiments, substituent groups useful for substituting unsaturated carbon atoms in the specified group or radical include —R^{a}, halo, —OR^{b}, —SR^{b}, —NR^{c}R^{c}, trihalomethyl, —CN, —S(O)₂OR^{b}, —C(O)R^{b}, —C(O)OR^{b}, —C(O)NR^{c}R^{c}, —OC(O)R^{b}, —OC(O)OR^{b}, —S(O)₂NR^{c}NR^{c}, —NR^{b}C(O)R^{b} and —NR^{b}C(O)OR^{b}, where R^{a}, R^{b} and R^{c} are as previously defined.

Substituent groups useful for substituting nitrogen atoms in heteroalkyl and cycloheteroalkyl groups include, —R^{a}, —O—, —OR^{b}, —SR^{b}, —S⁻, —NR^{c}R^{c}, trihalomethyl, —CF₃, —CN, —NO, —NO₂, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)OR^{b}, —OC(S)OR^{b}, —NR^{c}(O)R^{b}, —NR^{c}(S)R^{b}, —NR^{c}(O)OR^{b}, —NR^{b}C(S)OR^{b}, —NR^{b}C(O)NR^{c}R^{c}, —NR^{b}C(NR)R^{b} and —NR^{b}C(NR^{b})NR^{c}R^{c}, where R^{a}, R^{b} and R^{c} are as previously defined in the first embodiment of “substituted” above. In some embodiments, substituent groups useful for substituting nitrogen atoms in heteroalkyl, heteroalkenyl, cycloheteroalkyl and cycloheteroalkenyl groups include, R^{a}, —OR^{b}, —NR^{c}R^{c}, trihalomethyl, —CN, —S(O)₂OR^{b}, —OS(O)₂R^{b}, —OS(O)₂OR^{b}, —C(O)R^{b}, —C(NR^{b})R^{b}, —C(O)OR^{b}, —C(O)NR^{c}R^{c}, —OC(O)R^{b}, —OC(O)OR^{b}, —OS(O)₂NR^{c}NR^{c}, —NR^{c}(O)R^{b} and —NR^{b}C(O)OR^{b}, where R^{a}, R^{b} and R^{c} are as previously defined in the first embodiment of “substituted” above.

In some embodiments, substituent groups useful for substituting saturated carbon atoms in the specified group or radical include R^{a}, halo, —OR^{b}, —NR^{c}R^{c}, trihalomethyl, ═N—OR^{b}, —CN, —NR^{b}S(O)₂R^{b}, —C(O)R^{b}, —C(O)OR^{b}, —C(O)NR^{c}R^{c}, —OC(O)R^{b}, —OC(O)OR^{b}, —S(O)₂R^{b}, —S(O)₂NR^{c}NR^{c}, —OC(O)NR^{c}R^{c}, and —NR^{b}C(O)OR^{b}, substituent groups useful for substituting unsaturated carbon atoms in the specified group or radical include —R^{a}, halo, —OR^{b}, —SR^{b}, —NR^{c}R^{c}, trihalomethyl, —CN, —S(O)₂OR^{b}, —C(O)R^{b}, —C(O)OR^{b}, —C(O)NR^{c}R^{c}, —OC(O)R^{b}, —OC(O)OR^{b}, —S(O)₂NR^{c}NR^{c}, —NR^{b}C(O)R^{b} and —NR^{b}C(O)OR^{b} and substituent groups useful for substituting nitrogen atoms in heteroalkyl, heteroalkenyl, cycloheteroalkyl or cycloheteroalkenyl groups include, R^{a}, —OR^{b}, —NR^{c}R^{c}, trihalomethyl, —CN, —S(O)₂OR^{b}, —OS(O)₂R^{b}, —OS(O)₂OR^{b}, —C(O)R^{b}, —C(NR^{b})R^{b}, —C(O)OR^{b}, —C(O)NR^{c}R^{c}, —OC(O)R^{b}, —OC(O)OR^{b}, —OS(O)₂NR^{c}NR^{c}, —NR^{b}C(O)R^{b} and —NR^{b}C(O)OR^{b}, where each R^{a} is independently aryl, arylalkenyl, arylalkynyl, cycloalkyl, cycloalkenyl, cycloheteroalkyl, cycloheteroalkenyl, heteroalkyl, heteroalkenyl, heteroalkynyl, heteroaryl, heteroarylalkyl, heteroarylalkenyl or heteroarylalkynyl; R^{b} is independently hydrogen, alkyl, alkenyl, alkynyl, cycloalkyl, cycloalkenyl, cycloheteroalkyl, cycloheteroalkenyl, heteroalkyl, heteroalkenyl, substituted heteroalkynyl, arylalkyl, arylalkenyl, arylalkynyl, heteroarylalkyl, heteroarylalkenyl or heteroarylalkynyl and each R^{c} is independently R^{b} or alternatively, the two Res taken together with the nitrogen atom to which they are bonded form a 4-, 5-, 6 or -7 membered-cycloheteroalkyl or cycloheteroalkenyl ring.

The substituents used to substitute a specified group can in some embodiments, be further substituted, typically with one or more of the same or different groups selected from the various groups specified above.

“Subject,” “individual,” or “patient,” is used interchangeably herein and refers to a vertebrate, preferably a mammal. Mammals include, but are not limited to, murines, rodents, simians, humans, farm animals, sport animals and pets.

“Treating,” or “treatment,” of any disease or disorder refers, in some embodiments, to ameliorating the disease or disorder (i.e., arresting or reducing the development of the disease or at least one of the clinical symptoms thereof). Treatment may also be considered to include preemptive or prophylactic administration to ameliorate, arrest or prevent the development of the disease or at least one of the clinical symptoms. In a further feature the treatment rendered has lower potential for long-term side effects over multiple years. In other embodiments “treating” or “treatment” refers to ameliorating at least one physical parameter, which may not be discernible by the patient. In yet other embodiments, “treating” or “treatment” refers to inhibiting the disease or disorder, either physically. (e.g., stabilization of a discernible symptom), physiologically, (e.g., stabilization of a physical parameter) or both. In yet other embodiments, “treating” or “treatment” refers to delaying the onset of the disease or disorder.

“Therapeutically effective amount,” means the amount of a compound that, when administered to a patient for treating a disease, is sufficient to treat the disease. The “therapeutically effective amount” will vary depending on the compound, the disease and its severity and the age, weight, adsorption, distribution, metabolism and excretion etc., of the patient to be treated.

“Vehicle,” refers to a diluent, excipient or carrier with which a compound is administered to a subject. In some embodiments, the vehicle is pharmaceutically acceptable

### Compounds

Provided herein are compounds of Formula (I):

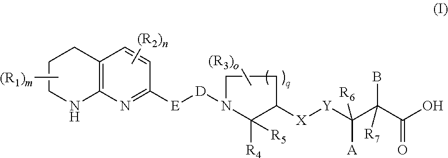

or pharmaceutically acceptable salts, hydrates or solvates thereof wherein:
each R₁ is independently hydrogen, alkyl, substituted alkyl, alkenyl, substituted alkenyl, alkynyl, substituted alkynyl, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl or substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl, substituted heteroarylalkynyl,
halo, —C(O)NR₁₉R₂₀, —C(O)OR₂₁, —NR₂₂C(O)OR₂₃, —NR₂₄C(O)OR₂₅, —OC(O)OR₂₆, —CN, —CF₃, —NR₂₇O₂R₁₇ or —OR₁₈; m is 0, 1, 2 or 3; each R₂ is independently hydrogen, alkyl, substituted alkyl, alkenyl, substituted alkenyl, alkynyl, substituted alkynyl, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl, substituted heteroarylalkynyl,
halo, —C(O)NR₁₉R₂₀, —C(O)OR²¹, —NR₂₂C(O)OR₂₃, —NR₂₄C(O)OR₂₅, —OC(O)OR₂₆, —CN, —CF₃, —NR₂₇SO₂R₂₈ or —OR₂₉; n is 0, 1 or 2; each R₃ is independently hydrogen, alkyl, substituted alkyl, alkenyl, substituted alkenyl, alkynyl, substituted alkynyl, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl, substituted heteroarylalkynyl,
halo, —C(O)NR₃₀R₃₁, —C(O)OR₃₂, —NR₃₃C(O)OR₃₄, —NR₃₅C(O)OR₃₆, —OC(O)OR₃₇, —CN, —CF₃, —NR₃₈SO₂R₃₉ or —OR⁴⁰; q is 0, 1, 2 or 3; o is 0, 1 or 2 when q is 0; o is 0, 1, 2 or 3 when q is 1; o is 0, 1, 2, 3 or 4 when q is 2; o is 0, 1, 2, 3, 4 or 5 when q is 3; R₄ is hydrogen, alkyl, substituted alkyl, alkenyl, substituted alkenyl, alkynyl, substituted alkynyl, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl, substituted heteroarylalkynyl, —F, —C(O)NR₄₁R₄₂, —C(O)R⁴³, —C(O)OR⁴⁴, —CN, —CF₃ or R₄ and R₅ together with the atom to which they are bonded form a C₄-C₈ cycloalkyl ring;
E is —CH₂— or —CH₂Z—; Z is —NR₄₆—, —S—, —SO₂— or —O—; D is —(CH₂)₂—, —(CH₂)₃—, —CH═CHCH₂—, —C(O)—, —C≡CCH₂—, phenyl, cyclohexyl or cyclopentyl when E is —CH₂—; D is —(CH₂)₂—, —(CH₂)₃—, —C(O)—, phenyl, cyclohexyl or cyclopentyl when Z is NR₄₅ or —O—; D is —(CH₂)₂—, —(CH₂)₃—, phenyl, cyclohexyl or cyclopentyl when Z is —SO₂— or —S—; X—Y is —C(O)NR₄₆—, —NR₄₇C(O)—, —C(O)O—, —CH₂CH₂—, —CH═CH—, —C≡C—, —NR₄₈CH₂—, —CH₂NR₄₉—, —O—CH₂—, —CH₂—O—, —SO₂NR₅₀—, —NR₅₁SO₂— or cyclopropyl; A is hydrogen, —OR₅₂, alkyl, substituted alkyl, alkenyl, substituted alkenyl, alkynyl, substituted alkynyl, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl, substituted heteroarylalkynyl or halo; B is hydrogen, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl, substituted heteroarylalkynyl, halo, —NR₅₃R₅₄, —O—R₅₅, —S—R₅₆ or —SO₂-R₅₁; R₈-R₅₃ and R₅₈-R₆₄ are independently hydrogen, alkyl, substituted alkyl, alkenyl, substituted alkenyl, alkynyl, substituted alkynyl, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl or substituted heteroarylalkynyl; R₅₄ is alkyl, substituted alkyl, alkenyl, substituted alkenyl, alkynyl, substituted alkynyl, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl, substituted heteroarylalkynyl, —C(O)R₅₈, —C(O)OR₅₉, —C(O)NR₆₀R₆₁, or —SO₂R₆₂;
R₅₅-R₅₇ are alkyl, substituted alkyl, alkenyl, substituted alkenyl, alkynyl, substituted alkynyl, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl or substituted heteroarylalkynyl; R₅ is hydrogen or —F; R is hydrogen, —F or —OR⁶³; and R₇; is hydrogen, —F or —OR₆₄; provided that R₅ is hydrogen when R₄ is —C(O)NR₄₁R₄₂, —C(O)R⁴³, —C(O)OR⁴⁴ or —CN; provided that A is not hydrogen, halo or —OR₅₂ when B is hydrogen or halo; provided that B is not hydrogen or halo when A is hydrogen, halo or —OR₅₂; provided that R₇ is hydrogen when B is halo, —NR₅₃R₅₄, —O—R₅₅, —S—R₅₆ or —SO₂R₅₇; provided that R₆ is —OR₆₃ only when X—Y are —CH₂CH₂—, —CH═CH—, —C≡C— or cyclopropyl; provided that A is not —OR⁵² when R₆ is —OR⁶³; and provided that A is not —Cl, —Br or —I when R₆ is —F.

In some embodiments, in compounds of Formula (I) each R₁ is independently hydrogen, alkyl, alkenyl, alkynyl, aryl, arylalkyl, arylalkenyl, arylalkynyl, cycloalkyl, cycloalkenyl, cycloheteroalkyl, cycloheteroalkenyl, heteroalkyl, heteroalkenyl, heteroalkynyl, heteroaryl, heteroarylalkyl, heteroarylalkenyl, heteroarylalkynyl,
halo, —C(O)NR₈R₉, —C(O)OR₁₀, —NR₁₁C(O)OR₁₂, —NR₁₃C(O)OR¹⁴, —OC(O)OR₁₅, —CN, —CF₃, —NR₁₆S O₂R₁₇ or —OR₁₈; m is 0, 1, 2 or 3; each R₂ is independently hydrogen, alkyl, alkenyl, alkynyl, aryl, arylalkyl, arylalkenyl, arylalkynyl, cycloalkyl, cycloalkenyl, cycloheteroalkyl, cycloheteroalkenyl, heteroalkyl, heteroalkenyl, heteroalkynyl, heteroaryl, heteroarylalkyl, heteroarylalkenyl, heteroarylalkynyl,
halo, —C(O)NR₁₉R₂₀, —C(O)OR²¹, —NR₂₂C(O)OR²³, —NR₂₄C(O)OR₂₅, —OC(O)OR²⁶, —CN, —CF₃, —NR₂₇SO₂R₂₈ or —OR₂₉; n is 0, 1 or 2; each R₃ is independently hydrogen, alkyl, alkenyl, alkynyl, aryl, arylalkyl, arylalkenyl, arylalkynyl, cycloalkyl, cycloalkenyl, substituted cycloalkenyl, cycloheteroalkyl, cycloheteroalkenyl, heteroalkyl, heteroalkenyl, heteroalkynyl, heteroaryl, heteroarylalkyl, heteroarylalkenyl, heteroarylalkynyl,
halo, —C(O)NR₃₀R₃₁, —C(O)OR₃₂, —NR₃₃C(O)OR₃₄, —NR₃₅C(O)OR₃₆, —OC(O)OR₃₇, —CN, —CF₃, —NR₃₈SO₂R₃₉ or —OR⁴⁰; q is 0, 1, 2 or 3; o is 0, 1 or 2 when q is 0; o is 0, 1, 2 or 3 when q is 1; o is 0, 1, 2, 3 or 4 when q is 2; o is 0, 1, 2, 3, 4 or 5 when q is 3; R₄ is hydrogen, alkyl, alkenyl, alkynyl, aryl, arylalkyl, arylalkenyl, arylalkynyl, cycloalkyl, cycloalkenyl, substituted cycloalkenyl, cycloheteroalkyl, cycloheteroalkenyl, heteroalkyl, heteroalkenyl, heteroalkynyl, heteroaryl, heteroarylalkyl, heteroarylalkenyl, heteroarylalkynyl,
halo, —C(O)NR₄₁R₄₂, —C(O)R⁴³, —C(O)OR⁴⁴, —CN, —CF₃ or R₄ and R₅ together with the atom to which they are bonded form a C₄-C₈ cycloalkyl ring;
R₅ is hydrogen, —F, —CF₃ or alkyl; E is —CH₂—, —CH₂Z—, Z is —NR₄₆—, —S—, —SO₂— or —O—; E is —CH₂—, —CH₂Z, Z is —NR₄₅—, —S—, —SO₂— or —O—; D is —(CH₂)₂—, —(CH₂)₃—, —CH═CHCH₂—, —C(O)—, —C≡CCH₂—, phenyl, cyclohexyl or cyclopentyl when E is —CH₂—; D is —(CH₂)₂—, —(CH₂)₃—, —C(O)—, phenyl, cyclohexyl or cyclopentyl when Z is NR₄₅ or —O—; D is —(CH₂)₂—, —(CH₂)₃—, phenyl, cyclohexyl or cyclopentyl when Z is —SO₂— or —S—;
X—Y is —C(O)NR₄₆—, —NR₄₇C(O)—, —C(O)O—, —CH₂CH₂—, —CH═CH—, —C≡C—, —NR₄₈CH₂—, —CH₂NR₄₉—, —O—CH₂—, —CH₂—O—, —SO₂NR₅₀—, —NR₅₁SO₂— or cyclopropyl; A is hydrogen, —OR₅₂, alkyl, alkenyl, alkynyl, aryl, arylalkyl, arylalkenyl, arylalkynyl, cycloalkyl, cycloalkenyl, cycloheteroalkyl, cycloheteroalkenyl, heteroalkyl, heteroalkenyl, heteroalkynyl, heteroaryl, heteroarylalkyl, heteroarylalkenyl, heteroarylalkynyl, or halo; B is hydrogen, aryl, arylalkyl, arylalkenyl, arylalkynyl, cycloalkyl, cycloalkenyl, cycloheteroalkyl, cycloheteroalkenyl, heteroalkyl, heteroalkenyl, heteroalkynyl, heteroaryl, heteroarylalkyl, heteroarylalkenyl, heteroarylalkynyl, halo, —NR₅₃R₅₄, —O—R₅₅, —S—R₅₆ or —SO₂-R₅₇; R₈-R₅₃ and R₅₈-R₆₄ are independently hydrogen, alkyl, alkenyl, alkynyl, aryl, arylalkyl, arylalkenyl, arylalkynyl, cycloalkyl, cycloalkenyl, cycloheteroalkyl, cycloheteroalkenyl, heteroalkyl, heteroalkenyl, heteroalkynyl, heteroaryl, heteroarylalkyl, aryl, arylalkyl, arylalkenyl, arylalkynyl, cycloalkyl, cycloalkenyl, cycloheteroalkyl, cycloheteroalkenyl, heteroalkyl, heteroalkenyl, heteroalkynyl, heteroaryl, heteroarylalkyl, heteroarylalkenyl, heteroarylalkynyl, —C(O)R₅₈, —C(O)OR⁵⁹, —C(O)NR₆₀R₆₁, or —SO₂R₆₂; R₅₅-R₅₇ are alkyl, alkenyl, alkynyl, aryl, arylalkyl, arylalkenyl, arylalkynyl, cycloalkyl, cycloalkenyl, cycloheteroalkyl, cycloheteroalkenyl, heteroalkyl, heteroalkenyl, heteroalkynyl, heteroaryl, heteroarylalkyl, heteroarylalkenyl or heteroarylalkynyl; R₅ is hydrogen or fluoro; R₆ is hydrogen, fluoro or —OR⁶³; and R₇ is hydrogen, fluoro or —OR₆₄.

In still other embodiments, in compounds of Formula (I) each R₁ is independently hydrogen, alkyl, alkenyl, aryl, arylalkyl, arylalkenyl, cycloalkyl, cycloalkenyl, cycloheteroalkyl, cycloheteroalkenyl, heteroalkyl, heteroalkenyl, heteroaryl, heteroarylalkyl, heteroarylalkenyl, halo, —C(O)NR₈R₉, —C(O)OR₁₀, —NR₁₁C(O)OR¹², —NR₁₃C(O)OR¹⁴, —OC(O)OR₁₅, —CN, —CF₃, —NR₁₆SO₂R₁₇ or —OR₁₈; m is 0 or 1; each R₂ is independently hydrogen, alkyl, alkenyl, alkynyl, aryl, arylalkyl, arylalkenyl, cycloalkyl, cycloalkenyl, cycloheteroalkyl, cycloheteroalkenyl, heteroalkyl, heteroalkenyl, heteroaryl, heteroarylalkyl, heteroarylalkenyl,
halo, —C(O)NR₁₉R₂₀, —C(O)OR₂₁, —NR₂₂C(O)OR²³, —NR₂₄C(O)OR₂₅, —OC(O)OR₂₆, —CN, —CF₃, —NR₂₇SO₂R₂₈ or —OR²⁹; n is 0, 1 or 2; each R₃ is independently hydrogen, alkyl, alkenyl, aryl, arylalkyl, arylalkenyl, cycloalkyl, cycloalkenyl, cycloheteroalkyl, cycloheteroalkenyl, heteroalkyl, heteroalkenyl, heteroalkynyl, heteroaryl, heteroarylalkyl, heteroarylalkenyl,
halo, —C(O)NR₃₀R₃₁, —C(O)OR₃₂, —NR₃₃C(O)OR₃₄, —NR₃₅C(O)OR₃₆, —OC(O)OR₃₇, —CN, —CF₃, —NR₃₈SO₂R₃₉ or —OR⁴⁰; q is 0, 1, 2 or 3; o is 0, 1 or 2 when q is 0; o is 0, 1, 2 or 3 when q is 1; o is 0, 1, 2, 3 or 4 when q is 2; o is 0, 1, 2, 3, 4 or 5 when q is 3; R₄ is hydrogen, alkyl, alkenyl, aryl, arylalkyl, arylalkenyl, arylalkynyl, cycloalkyl, cycloalkenyl, cycloheteroalkyl, cycloheteroalkenyl, heteroalkyl, heteroalkenyl, heteroaryl, heteroarylalkyl, heteroarylalkenyl,
halo, —C(O)NR₄₁R₄₂, —C(O)R⁴³, —C(O)OR⁴⁴, —CN, —CF₃ or R₄ and R₅ together with the atom to which they are bonded form a C₄-C₈ cycloalkyl ring;
R₅ is hydrogen, —F, —CF₃ or alkyl; E is —CH₂—, —CH₂Z—, Z is —NR₄₆—, —S—, —SO₂— or —O—; E is —CH₂—, —CH₂Z, Z is —NR₄₅—, —S—, —SO₂— or —O—; D is —(CH₂)₂—, —(CH₂)₃—, —CH═CHCH₂—, —C(O)—, —C≡CCH₂—, phenyl, cyclohexyl or cyclopentyl when E is —CH₂—; D is —(CH₂)₂—, —(CH₂)₃—, —C(O)—, phenyl, cyclohexyl or cyclopentyl when Z is NR₄₅ or —O—; D is —(CH₂)₂—, —(CH₂)₃—, phenyl, cyclohexyl or cyclopentyl when Z is —SO₂— or —S—;
X—Y is —C(O)NR₄₆—, —NR₄₇C(O)—, —C(O)O—, —CH₂CH₂—, —CH═CH—, —C≡C—, —NR₄₈CH₂—, —CH₂NR₄₉—, —O—CH₂—, —CH₂—O—, —SO₂NR₅₀—, —NR₅₁SO₂— or cyclopropyl; A is hydrogen, —OR₅₂, alkyl, alkenyl, aryl, arylalkyl, arylalkenyl, cycloalkyl, cycloalkenyl, cycloalkenyl, cycloheteroalkyl, cycloheteroalkenyl, heteroalkyl, heteroalkenyl, heteroalkynyl, heteroaryl, heteroarylalkyl, heteroarylalkenyl, or halo; B is hydrogen, aryl, arylalkyl, arylalkenyl, arylalkynyl, cycloalkyl, cycloalkenyl, cycloheteroalkyl, cycloheteroalkenyl, heteroalkyl, heteroalkenyl, heteroalkynyl, heteroaryl, heteroarylalkyl, heteroarylalkenyl, halo, —NR₅₃R₅₄, —O—R₅₅, —S—R₅₆ or —SO₂-R₅₇; R₈-R₅₃ and R₅₈-R₆₄ are independently hydrogen, alkyl, alkenyl, aryl, arylalkyl, arylalkenyl, cycloalkyl, cycloalkenyl, cycloheteroalkyl, cycloheteroalkenyl, heteroalkyl, heteroalkenyl, heteroaryl, heteroarylalkyl, aryl, arylalkyl, arylalkenyl, cycloalkyl, cycloalkenyl, cycloheteroalkyl, cycloheteroalkenyl, heteroalkyl, heteroalkenyl, heteroaryl, heteroarylalkyl, heteroarylalkenyl, heteroarylalkynyl, —C(O)R₅₅, —C(O)OR⁵⁹, —C(O)NR₆₀R₆₁, or —SO₂R₆₂; R₅₅-R₅₇ are alkyl, alkenyl, aryl, arylalkyl, arylalkenyl, cycloalkyl, cycloalkenyl, cycloheteroalkyl, cycloheteroalkenyl, heteroalkyl, heteroalkenyl, heteroaryl, heteroarylalkyl or heteroarylalkenyl; R₅ is hydrogen or fluoro; R₆ is hydrogen, fluoro or —OR₆₃; and R₇ is hydrogen, fluoro or —OR₆₄.

In still other embodiments, in compounds of Formula (I) each R₁ is independently hydrogen, alkyl, alkenyl, aryl, arylalkyl, arylalkenyl, cycloalkyl, cycloalkenyl, cycloheteroalkyl, cycloheteroalkenyl, heteroalkyl, heteroalkenyl, heteroaryl, heteroarylalkyl, heteroarylalkenyl, halo, —C(O)NR₈R₉, —C(O)OR₁₀, —NR₁₁C(O)OR₁₂, —NR₁₃C(O)OR₁₄, —OC(O)OR₁₅, —CN, —CF₃, —NR₁₆S O₂R₁₇ or —OR₁₈, m is 0 or 1; each R₂ is independently hydrogen, alkyl, alkenyl, aryl, arylalkyl, arylalkenyl, cycloalkyl, cycloalkenyl, cycloheteroalkyl, cycloheteroalkenyl, heteroalkyl, heteroalkenyl, heteroaryl, heteroarylalkyl, heteroarylalkenyl,
halo, —C(O)NR₁₉R₂₀, —C(O)OR₂₁, —NR₂₂C(O)OR²³, —NR₂₄C(O)OR₂₅, —OC(O)OR₂₆, —CN, —CFs, —NR₂₇SO₂R₂₈ or —OR²⁹; n is 0, 1 or 2; each R₃ is independently hydrogen, alkyl, alkenyl, aryl, arylalkyl, arylalkenyl, cycloalkyl, cycloalkenyl, cycloheteroalkyl, cycloheteroalkenyl, heteroalkyl, heteroalkenyl, heteroaryl, heteroarylalkyl, heteroarylalkenyl,
halo, —C(O)NR₃₀R₃₁, —C(O)OR₃₂, —NR₃₃C(O)OR₃₄, —NR₃₅C(O)OR₃₆, —OC(O)OR₃₇, —CN, —CF₃, —NR₃₈SO₂R₃₉ or —OR₄₀; q is 0, 1, 2 or 3; o is 0, 1 or 2 when q is 0; o is 0, 1, 2 or 3 when q is 1; o is 0, 1, 2, 3 or 4 when q is 2; o is 0, 1, 2, 3, 4 or 5 when q is 3; R₄ is hydrogen, alkyl, alkenyl, aryl, arylalkyl, arylalkenyl, cycloalkyl, cycloalkenyl, cycloheteroalkyl, cycloheteroalkenyl, heteroalkyl, heteroalkenyl, heteroaryl, heteroarylalkyl, heteroarylalkenyl,
halo, —C(O)NR₄₁R₄₂, —C(O)R⁴³, —C(O)OR₄₄, —CN, —CF₃ or R₄ and R₅ together with the atom to which they are bonded form a C₄-C₈ cycloalkyl ring;
R₅ is hydrogen, —F, —CF₃ or alkyl; E is —CH₂—, —CH₂Z—, Z is —NR₄₆—, —S—, —SO₂— or —O—; E is —CH₂—, —CH₂Z, Z is —NR₄₅—, —S—, —SO₂— or —O—; D is —(CH₂)₂—, —(CH₂)₃—, —CH═CHCH₂—, —C(O)—, —C≡CCH₂—, phenyl, cyclohexyl or cyclopentyl when E is —CH₂—; D is —(CH₂)₂—, —(CH₂)₃—, —C(O)—, phenyl, cyclohexyl or cyclopentyl when Z is NR₄₅ or —O—; D is —(CH₂)₂—, —(CH₂)₃—, phenyl, cyclohexyl or cyclopentyl when Z is —SO₂— or —S—; X—Y is —C(O)NR₄₆—, —NR₄₇C(O)—, —C(O)O—, —CH₂CH₂—, —CH═CH—, —C≡C—, —NR₄₈CH₂—, —CH₂NR₄₉—, —O—CH₂—, —CH₂—O—, —SO₂NR₅₀—, —NR₅₁SO₂— or cyclopropyl; A is hydrogen, —OR₅₂, alkyl, alkenyl, aryl, arylalkyl, arylalkenyl, cycloalkyl, cycloalkenyl, cycloalkenyl, cycloheteroalkyl, cycloheteroalkenyl, heteroalkyl, heteroalkenyl, heteroaryl, heteroarylalkyl, heteroarylalkenyl, or halo; B is hydrogen, aryl, arylalkyl, arylalkenyl, arylalkynyl, cycloalkyl, cycloalkenyl, cycloheteroalkyl, cycloheteroalkenyl, heteroalkyl, heteroalkenyl, heteroaryl, heteroarylalkyl, heteroarylalkenyl, halo, —NR₅₃R₅₄, —O—R₅₅, —S—R₅₆ or —SO₂-R₅₇; R₈-R₅₃ and R₅₈-R₆₄ are independently hydrogen, alkyl, alkenyl, aryl, arylalkyl, arylalkenyl, cycloalkyl, cycloalkenyl, cycloheteroalkyl, cycloheteroalkenyl, heteroalkyl, heteroalkenyl, heteroaryl, heteroarylalkyl, aryl, arylalkyl, arylalkenyl, cycloalkyl, cycloalkenyl, cycloheteroalkyl, cycloheteroalkenyl, heteroalkyl, heteroalkenyl, heteroaryl, heteroarylalkyl, heteroarylalkenyl, —C(O)R₅₈, —C(O)OR₅₉, —C(O)NR₆₀R₆₁, or —SO₂R₆₂; R₅₅-R₅₇ are alkyl, alkenyl, aryl, arylalkyl, arylalkenyl, cycloalkyl, cycloalkenyl, cycloheteroalkyl, cycloheteroalkenyl, heteroalkyl, heteroalkenyl, heteroaryl, heteroarylalkyl or heteroarylalkenyl; R₅ is hydrogen or fluoro; R₆ is hydrogen, fluoro or —OR₆₃; and R₇ is hydrogen, fluoro or —OR₆₄.

In still other embodiments, in compounds of Formula (I) each R₁ is independently hydrogen, alkyl, aryl, arylalkyl, cycloalkyl, cycloheteroalkyl, heteroalkyl, heteroaryl, halo, —C(O)NR₈R₉, —C(O)OR₁₀, —NR₁₁C(O)OR₁₂, —NR₁₃C(O)OR₁₄, —OC(O)OR₁₅, —CN, —CF₃, —NR₁₆S O₂R₁₇ or —OR₁₈; m is 0 or 1; each R₂ is independently hydrogen, alkyl, aryl, arylalkyl, cycloalkyl, cycloheteroalkyl, heteroalkyl, heteroaryl, heteroarylalkyl,
halo, —C(O)NR₁₉R₂₀, —C(O)OR²¹, —NR₂₂C(O)OR²³, —NR₂₄C(O)OR₂₅, —OC(O)OR₂₆, —CN, —CF₃, —NR₂₇SO₂R₂₈ or —OR²⁹; n is 0, 1 or 2, each R₃ is independently hydrogen, alkyl, alkenyl, aryl, arylalkyl, cycloalkyl, cycloheteroalkyl, heteroalkyl, heteroalkynyl, heteroaryl, heteroarylalkyl,
halo, —C(O)NR₃₀R₃₁, —C(O)OR₃₂, —NR₃₃C(O)OR₃₄, —NR₃SC(O)OR₃₆, —OC(O)OR³⁷, —CN, —CF₃, —NR₃₈SO₂R₃₉ or —OR⁴⁰; q is 0, 1, 2 or 3; o is 0, 1 or 2 when q is 0; o is 0, 1, 2 or 3 when q is 1; o is 0, 1, 2, 3 or 4 when q is 2; o is 0, 1, 2, 3, 4 or 5 when q is 3; R₄ is hydrogen, alkyl, alkenyl, aryl, arylalkyl, cycloalkyl, cycloheteroalkyl, cycloheteroalkenyl, heteroalkyl, heteroaryl, heteroarylalkyl, halo, —C(O)NR₄₁R₄₂, —C(O)R⁴³, —C(O)OR⁴⁴, —CN, —CF₃ or R₄ and R₅ together with the atom to which they are bonded form a C₄-C₈ cycloalkyl ring;
R₅ is hydrogen, —F, —CF₃ or alkyl; E is —CH₂—, —CH₂Z—, Z is —NR₄₆—, —S—, —SO₂— or —O—; E is —CH₂—, —CH₂Z, Z is —NR₄₅—, —S—, —SO₂— or —O—; D is —(CH₂)₂—, —(CH₂)₃—, —CH═CHCH₂—, —C(O)—, —C≡CCH₂—, phenyl, cyclohexyl or cyclopentyl when E is —CH₂—; D is —(CH₂)₂—, —(CH₂)₃—, —C(O)—, phenyl, cyclohexyl or cyclopentyl when Z is NR₄₅ or —O—; D is —(CH₂)₂—, —(CH₂)₃—, phenyl, cyclohexyl or cyclopentyl when Z is —SO₂— or —S—; X—Y is —C(O)NR₄₆—, —NR₄₇C(O)—, —C(O)O—, —CH₂CH₂—, —CH═CH—, —C≡C—, —NR₄₈CH₂—, —CH₂NR₄₉—, —O—CH₂—, —CH₂—O—, —SO₂NR₅₀—, —NR₅₁SO₂— or cyclopropyl; A is hydrogen, —OR₅₂, alkyl, alkenyl, aryl, arylalkyl, cycloalkyl, cycloalkenyl, cycloheteroalkyl, heteroalkyl, heteroalkenyl, heteroaryl, heteroarylalkyl or halo; B is hydrogen, aryl, arylalkyl, cycloalkyl, cycloheteroalkyl, heteroalkyl, heteroaryl, heteroarylalkyl, halo, —NR₅₃R₅₄, —O—R₅₅, —S—R₅₆ or —SO₂-R₅₇; R₈-R₅₃ and R₅₈-R₆₄ are independently hydrogen, alkyl, aryl, arylalkyl, cycloalkyl, cycloheteroalkyl, heteroalkyl, heteroaryl, aryl, arylalkyl, cycloalkyl, cycloheteroalkyl, heteroalkyl, heteroaryl,
heteroarylalkyl, —C(O)R₅₈, —C(O)OR₅₉, —C(O)NR₆₀R₆₁, or —SO₂R₆₂; R₅₅-R₅₇ are alkyl, aryl, arylalkyl, cycloalkyl, cycloheteroalkyl, heteroalkyl, heteroaryl or heteroarylalkyl; R₅ is hydrogen or fluoro; R₆ is hydrogen, fluoro or —OR⁶³; and R₇ is hydrogen, fluoro or —OR₆₄.

In some embodiments, a compound of Formula (II) is provided:

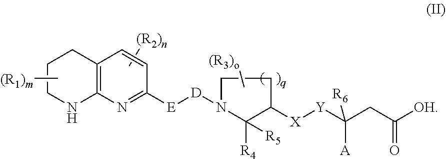

In other embodiments, a compound of Formula (III) is provided:

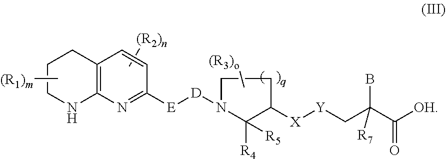

In still other embodiments, a compound of Formula (IV) is provided:

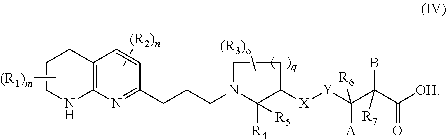

In still other embodiments, compound of Formula (V) is provided:

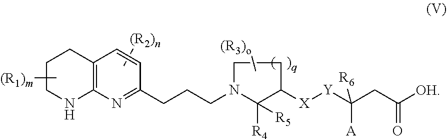

In still other embodiments, compound of Formula (VI) is provided:

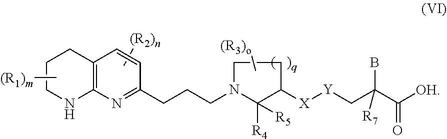

In some embodiments of compounds of Formulae (I-VI), each R₁ is independently alkyl, substituted alkyl, alkenyl, substituted alkenyl, phenyl, substituted phenyl, cycloalkyl, heteroalkyl, cycloheteroalkyl, —F, —C(O)NR₈R₉, —C(O)OR₁₀, —OC(O)OR₁₅, —CF₃, or —OR₁₅. In other embodiments, each R₁ is independently (C₁-C₄) alkyl, (C₂-C₄) alkenyl, phenyl, substituted phenyl, (C₅-C₇) cycloalkyl, (C₅-C₇) cycloheteroalkyl, —F, or —CF₃.

In some embodiments of compounds of Formulae (I-VI), m is 0 or 1. In other embodiments, n is 0 or 1.

In some embodiments of compounds of Formulae (I-VI), each R₂ is independently alkyl, substituted alkyl, alkenyl, substituted alkenyl, phenyl, substituted phenyl, cycloalkyl, substituted cycloalkyl, cycloalkenyl, substituted cycloalkenyl, heteroalkyl, cycloheteroalkyl or cycloheteroalkenyl,
halo, —C(O)NR₁₉R₂₀, —C(O)OR²¹, —NR₂₂C(O)OR²³, —NR₂₄C(O)OR₂₅, —OC(O)OR²⁶, —CN, —CF₃, —NR₂₇SO₂R₂₈ or —OR²⁹. In other embodiments, each R₂ is independently (C₁-C₄) alkyl, (C₂-C₄) alkenyl, phenyl, substituted phenyl, (C₅-C₇) cycloalkyl, (C₅-C₇) cycloheteroalkyl, halo, C(O)NR₁₉R₂₀, —C(O)OR²¹, —NR₂₂C(O)OR²³, —NR₂₄C(O)OR₂₅, —OC(O)OR₂₆, —CN, —CF₃, —NR₂₇SO₂R₂₈ or —OR²⁹.

In some embodiments of compounds of Formulae (I-VI), each R₃ is independently, alkyl, substituted alkyl, alkenyl, substituted alkenyl, phenyl, substituted phenyl, cycloalkyl, heteroalkyl, cycloheteroalkyl, —F, —C(O)NR₃₀R₃₁, —C(O)OR₃₂, —OC(O)OR₃₇, —CF₃, or —OR₄₀. In other embodiments, each R₃ is independently (C₁-C₄) alkyl, (C₂-C₄) alkenyl, phenyl, substituted phenyl, (C₅-C₇) cycloalkyl, (C₅-C₇) cycloheteroalkyl, —F, or —CF₃.

In some embodiments of compounds of Formulae (I-VI), o is 0 or 1. In other embodiments, o is 0, 1, 2 or 3. In some embodiments, o is 1, 2, or 3. In some embodiments, o is 1 or 2.

In some embodiments of compounds of Formulae (I-VI), R₄ is hydrogen, alkyl, substituted alkyl, alkenyl, substituted alkenyl, —F, —C(O)NR₄₁R₄₂, —C(O)R₄₃, —C(O)OR₄₄ or —CF₃. In other embodiments of compounds of Formulae (I-VI), R₄ is hydrogen, (C₁-C₄) alkyl, (C₂-C₄) alkenyl, —F or —CF₃.

In some embodiments of Formulae (I-VI), R₅ is hydrogen or —F.

In some embodiments of compounds of Formulae (I-VI), R₈-R₅₃ and R₅₈-R₆₄ are independently hydrogen, alkyl, alkenyl, aryl, substituted aryl, arylalkyl, substituted arylalkyl, cycloalkyl, substituted cycloalkyl, heteroalkyl, heteroalkenyl, heteroaryl, substituted heteroaryl, cycloheteroalkyl or substituted cycloheteroalkyl. In other embodiments, R₈-R₅₃ and R₅₈-R₆₄ are independently hydrogen, (C₁-C₄) alkyl, aryl, substituted aryl, cycloalkyl, substituted cycloalkyl, heteroaryl, substituted heteroaryl, cycloheteroalkyl or substituted cycloheteroalkyl.

In some embodiments of compounds of Formulae (I-VI), R₅₅-R₅₇ are independently alkyl, alkenyl, aryl, substituted aryl, arylalkyl, substituted arylalkyl, cycloalkyl, substituted cycloalkyl, heteroalkyl, heteroalkenyl, heteroaryl, substituted heteroaryl, cycloheteroalkyl or substituted cycloheteroalkyl. In other embodiments, R₅₅-R₅₇ are independently (C₁-C₄) alkyl, aryl, substituted aryl, cycloalkyl, substituted cycloalkyl, heteroaryl, substituted heteroaryl, cycloheteroalkyl or substituted cycloheteroalkyl.

In some embodiments of compounds of Formulae (I-VI), each R₁ is independently alkyl, substituted alkyl, alkenyl, substituted alkenyl, phenyl, substituted phenyl, cycloalkyl, heteroalkyl, cycloheteroalkyl, —F, —C(O)NR₈R₉, —C(O)OR₁₀, —OC(O)OR₁₅, —CF₃, or —OR₁₅, m is 0 or 1, each R₂ is independently alkyl, substituted alkyl, alkenyl, substituted alkenyl, phenyl, substituted phenyl, cycloalkyl, substituted cycloalkyl, cycloalkenyl, substituted cycloalkenyl, heteroalkyl, cycloheteroalkyl,
halo, —C(O)NR₁₉R₂₀, —C(O)OR²¹, —NR₂₂C(O)OR²³, —NR₂₄C(O)OR₂₅, —OC(O)OR²⁶, —CN, —CF₃, —NR₂₇SO₂R₂₈ or —OR²⁹, n is 0 or 1, each R₃ is independently, alkyl, substituted alkyl, alkenyl, substituted alkenyl, phenyl, substituted phenyl, cycloalkyl, heteroalkyl, cycloheteroalkyl, —F, —C(O)NR₃₀R₃₁, —C(O)OR₃₂, —OC(O)OR₃₇, —CF₃, or —OR₄₀, o is 0, 1, 2 or 3, R₄ is hydrogen, alkyl, substituted alkyl, alkenyl, substituted alkenyl, —F, —C(O)NR₄₁R₄₂, —C(O)OR⁴³, —C(O)OR⁴⁴ or —CF₃ and R₈-R₅₃ and R₅₈-R₆₄ are independently hydrogen, alkyl, alkenyl, aryl, substituted aryl, arylalkyl, substituted arylalkyl, cycloalkyl, substituted cycloalkyl, heteroalkyl, heteroalkenyl, heteroaryl, substituted heteroaryl, cycloheteroalkyl or substituted cycloheteroalkyl. In other embodiments, R₁ is independently (C₁-C₄) alkyl, (C₂-C₄) alkenyl, phenyl, substituted phenyl, (C₅-C₇) cycloalkyl, (C₅-C₇) cycloheteroalkyl, —F, or —CF₃, m is 0 or 1, each R₂ is independently (C₁-C₄) alkyl, (C₂-C₄) alkenyl, phenyl, substituted phenyl, (C₅-C₇) cycloalkyl, (C₅-C₇) cycloheteroalkyl, halo, C(O)NR₁₉R₂₀, —C(O)OR₂₁, —NR₂₂C(O)OR₂₃, —NR₂₄C(O)OR₂₅, —OC(O)OR₂₆, —CN, —CF₃, —NR₂₇SO₂R₂₈ or —OR²⁹, is 0 or 1, each R₃ is independently (C₁-C₄) alkyl, (C₂-C₄) alkenyl, phenyl, substituted phenyl, (C₅-C₇) cycloalkyl, (C₅-C₇) cycloheteroalkyl, —F, or —CF₃, o is 0 or 1, R₄ is hydrogen, C₁-C₄) alkyl, (C₂-C₄) alkenyl, —F or —CF₃ and R₈-R₅₃ and R₅₈-R₆₄ are independently hydrogen, (C₁-C₄) alkyl, aryl, substituted aryl, cycloalkyl, substituted cycloalkyl, heteroaryl, substituted heteroaryl, cycloheteroalkyl or substituted cycloheteroalkyl.

In some embodiments of compounds of Formulae (I, II, IV and V), A is aryl, substituted aryl, arylalkyl, substituted arylalkyl, arylalkenyl, substituted arylalkenyl, cycloalkyl, substituted cycloalkyl, cycloalkenyl, substituted cycloalkenyl, heteroaryl, substituted heteroaryl, heteroarylalkyl, substituted heteroarylalkyl, heteroarylalkenyl, substituted heteroarylalkenyl, cycloheteroalkyl, substituted cycloheteroalkyl, cycloheteroalkenyl or substituted cycloheteroalkenyl. In other embodiments, A is aryl, substituted aryl, arylalkyl, substituted arylalkyl, heteroaryl, substituted heteroaryl, heteroarylalkyl or substituted heteroarylalkyl. In still other embodiments of compounds of Formulae (I, II. IV and V), A is aryl, substituted aryl, heteroaryl or substituted heteroaryl.

In some embodiments of compounds of Formulae (I, III, IV and VI), B is aryl, substituted aryl, arylalkyl, substituted arylalkyl, arylalkenyl, substituted arylalkenyl, cycloalkyl, substituted cycloalkyl, cycloalkenyl, substituted cycloalkenyl, heteroaryl, substituted heteroaryl, heteroarylalkyl, substituted heteroarylalkyl, heteroarylalkenyl, substituted heteroarylalkenyl, cycloheteroalkyl, substituted cycloheteroalkyl, cycloheteroalkenyl or substituted cycloheteroalkenyl. In other embodiments. B is aryl, substituted aryl, arylalkyl, substituted arylalkyl, heteroaryl, substituted heteroaryl, heteroarylalkyl or substituted heteroarylalkyl. In still other embodiments, B is aryl, substituted aryl, heteroaryl or substituted heteroaryl. In still other embodiments, B is —NR₅₃R₅₄. In still other embodiments, B is —NHR₅₄, R₅₄ is aryl, substituted aryl, arylalkyl, substituted arylalkyl, heteroaryl, substituted heteroaryl, heteroarylalkyl or substituted heteroarylalkyl —C(O)R₅₈, —C(O)OR₅₉, or —SO₂R₆₂. In still other embodiments, B is —NHR₅₄ and R₅₄ is aryl, substituted aryl, heteroaryl, substituted heteroaryl, —C(O)R₅₈, —C(O)OR₅₉, or —SO₂R₆₂.

In some embodiments of compounds of Formulae (I) and (IV). A is aryl, substituted aryl, heteroaryl or substituted heteroaryl and B is aryl, substituted aryl, heteroaryl or substituted heteroaryl. In other embodiments of compounds of Formulae (I) and (IV), A is aryl, substituted aryl, heteroaryl or substituted heteroaryl and B is —NR₅₃R₅₄. In still other embodiments of compounds of Formulae (I) and (IV). A is aryl, substituted aryl, heteroaryl or substituted heteroaryl and B is —NHR₅₄, R₅₄ is aryl, substituted aryl, arylalkyl, substituted arylalkyl, heteroaryl, substituted heteroaryl, heteroarylalkyl or substituted heteroarylalkyl —C(O)R₅₈, —C(O)OR₅₉, or —SO₂R₆₂. In still other embodiments of compounds of Formulae (I) and (IV), A is aryl, substituted aryl, heteroaryl or substituted heteroaryl and B is —NHR₅₄ and R₅₄ is aryl, substituted aryl, heteroaryl, substituted heteroaryl, —C(O)R₅₈, —C(O)OR₅₉, or —SO₂R₆₂.

In some embodiments, a compound of Formula (VII) is provided:

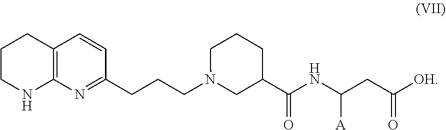

In some embodiments, A is aryl, substituted aryl, heteroaryl or substituted heteroaryl. In other embodiments, A is aryl, substituted phenyl, heteroaryl or substituted heteroaryl.

In some embodiments a compound of Formula (VIIII) is provided:

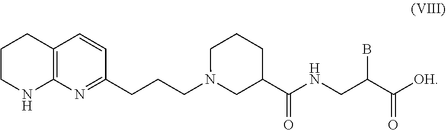

In some embodiments, B is aryl, substituted aryl, heteroaryl or substituted heteroaryl. In other embodiments, B is aryl, substituted phenyl, heteroaryl or substituted heteroaryl. In still other embodiments B is —NHR₅₄ and R₅₄ is aryl, substituted aryl, heteroaryl, substituted heteroaryl, —C(O)R₅₅, —C(O)OR₅₉, or —SO₂R₆₂.

In some embodiments, a compound of Formula (IX) is provided:

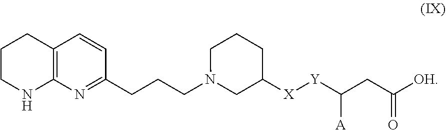

In some embodiments, A is phenyl or substituted phenyl.

In some embodiments, in a compound of Formula (Ia),

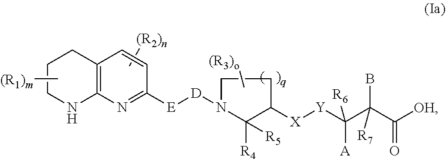

or a pharmaceutically acceptable salt thereof; wherein:
q is 1, 2 or 3;
R¹ and R³ are each independently selected at each occurrence from halogen, C₁₋₄ alkyl, C₁₋₄-haloalkyl, —OR¹¹, —SR¹¹, —N(R¹¹)₂, —C(O)N(R¹¹)₂, —C(O)OR¹¹, ═O, ═S, and —CN;
m is selected from 0, 1, 2, 3, 4, 5, and 6;
o is selected from 0, 1, 2, 3, 4, 5, 6, 7, and 8;
R² is independently selected at each occurrence from halogen, C₁₋₄ alkyl, C₁₋₄haloalkyl, —OR¹², —SR¹², —N(R²)₂, —CN, and —NO₂;
n is 0, 1 or 2;
R⁴ and R⁵ are each independently selected from hydrogen, halogen, C₁₋₄ alkyl, C₁₋₄ haloalkyl, —OR¹³, —SR¹³, —N(R¹³)₂, and —CN; or R⁴ and R⁵ are taken together to form a double bonded substituent selected from ═O, ═S and ═N(R¹³);
D is selected from a bond, —C(O)—, —C≡CCH₂—, and —CH═CHCH₂—,
E is selected from C₁₋₄ alkylene and —(CH₂)Z—,
   wherein Z is selected from —NH—, —S—, —SO₂—, and —O—;
X—Y is selected from: ^{λ}—C(O)N(R¹⁴)—, ^{λ}—N(R¹⁴)C(O)—, ^{λ}—N(R¹⁴)C(O)C(R¹⁵)₂—, ^{λ}—C(O)O—, ^{λ}—C(R¹⁵)₂C(R¹⁵)₂—, ^{λ}—CH═CH—, ^{λ}—C≡C—, ^{λ}—N(R¹⁸)C(R¹⁵)₂—, ^{λ}—C(R¹⁵)₂—, ^{λ}—N(R¹⁴)—, ^{λ}—O—, ^{λ}—OC(R¹⁵)₂—, ^{λ}—C(R¹⁵)₂O—, ^{λ}—SO₂N(R¹⁴)—, and ^{λ}—N(R¹⁴)SO₂—;
   wherein ^{λ} denotes the attachment of X—Y to

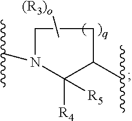

R⁶ and R⁷ are each independently selected at each occurrence from:
   hydrogen, halogen, C₁₋₄ alkyl, C₁₋₄ haloalkyl, —OR¹⁶, and —CN;
A is selected from (i) and (ii):
   (i) hydrogen, halogen, and —CN, or A and R⁶ come together to form a C₃₋₆ carbocycle or 3- to 6-membered heterocycle;
   (ii) —OR¹⁷, —SR¹⁷, —N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —C(O)R¹⁷, —C(O)OR¹⁷, —OC(O)R¹⁷, —OC(O)N(R¹⁷)₂, —C(O)N(R¹⁷)₂, —S(O)R¹⁷, —S(O)₂R¹⁷, and —S(O)₂N(R¹⁷)₂;
      C₁₋₆ alkyl optionally substituted with one or more substituents independently selected from:
         halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═N(R¹⁷), —N₃, and —CN, C₃₋₁₀ carbocycle, and 3- to 10-membered heterocycle,
         wherein the C₃₋₁₀ carbocycle and 3- to 10-membered heterocycle are each optionally substituted with one or more substituents independently selected from halogen, C₁₋₆ alkyl, C₁₋₆ haloalkyl, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂—N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═N(R¹⁷), —N₃, and —CN; and
         C₃₋₁₂ carbocycle and 3- to 12-membered heterocycle, any of which is optionally substituted with one or more substituents independently selected from:
         halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R′, —S(O)₂N(R¹⁷)₂, —NO₂, ═O, ═S, ═N(R¹⁷), —N₃, and —CN;
         C₁₋₆ alkyl optionally substituted with one or more substituents independently selected from halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═N(R¹⁷), —N₃, and —CN; and
         C₃₋₁₀ carbocycle and 3- to 10-membered heterocycle, any of which is optionally substituted with one or more substituents independently selected from halogen, C₁₋₄ alkyl, C₁₋₄haloalkyl, and ═O;
B is selected from (I) when A is selected from (ii), or B is selected from (II) when A is selected from (i):
   (I) hydrogen, halogen, and —CN, or B and R⁷ are taken together to form a C₃₋₆ carbocycle or a 3- to 6-membered heterocycle;
   (II) —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂—N(R¹⁸)C(S)N(R¹⁸)₂, —N(R¹⁸)S(O)₂(R¹⁸), —S(O)R¹⁸, —S(O)₂R¹⁸, and —S(O)₂N(R¹⁸)₂;
      C₁₋₆ alkyl optionally substituted with one or more substituents independently selected from:
         halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸. —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═N(R¹⁸), —N₃, —CN, C₃₋₁₀ carbocycle and 3- to 10-membered heterocycle,
         wherein the C₃₋₁₀ carbocycle and 3- to 10-membered heterocycle are optionally substituted with one or more substituents independently selected from halogen, C₁₋₆ alkyl, C₁₋₆ haloalkyl, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═N(R¹⁸), —N₃, and —CN; and
      C₃₋₁₂ carbocycle and 3- to 12-membered heterocycle, any of which is optionally substituted with one or more substituents independently selected from:
         C₃₋₆₆ carbocycle and 3- to 6-membered heterocycle, any of which is optionally substituted with one or more substituents independently selected from: halogen, C₁₋₄ alkyl, C₁₋₄haloalkyl, —OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —N(R²¹)C(O)OR²¹, —N(R²¹)C(O)N(R²¹)₂, —N(R²¹)C(S)N(R²¹)₂, —N(R²¹)S(O)₂(R²¹), —S(O)R²¹, —S(O)₂R²¹, —S(O)₂N(R²)₂, —NO₂, ═O, ═S, ═N(R²¹), —N₃, and —CN;
R¹¹, R¹², R¹³, R¹⁴, and R₁₆ are each independently selected at each occurrence from hydrogen, C₁₋₄ alkyl, and C₁₋₄ haloalkyl;
R¹⁵ is independently selected at each occurrence from hydrogen, halogen, C₁₋₄ alkyl, and C₁₋₄ haloalkyl;
R¹⁷ is independently selected at each occurrence from:
   hydrogen;
   C₁₋₆ alkyl optionally substituted with one or more substituents independently selected from: halogen, —OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —NO₂, ═O, ═S, ═N(R²¹), —N₃, and —CN; and
   C₃₋₆ carbocycle and 3- to 6-membered heterocycle, any of which is optionally substituted with one or more substituents independently selected from: halogen, C₁₋₄ alkyl, C₁₋₄ haloalkyl, —OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —N(R²¹)C(O)OR²¹, —N(R²¹)C(O)N(R¹¹)₂, —N(R²¹)C(S)N(R²¹)₂, —N(R²¹)S(O)₂(R¹¹), —S(O)R²¹, —S(O)₂R²¹, —S(O)₂N(R²¹)₂, —NO₂, ═O, ═S, ═N(R²¹), —N₃, and —CN;
R¹⁸ is independently selected at each occurrence from:
   hydrogen;
   C₁₋₆ alkyl optionally substituted with one or more substituents independently selected from:
      halogen, —OR²², —SR²², —N(R²²)₂, —C(O)R²², —C(O)OR²², —OC(O)R²², —OC(O)N(R²²)₂, —C(O)N(R²²)₂, —N(R²²)C(O)R²², —NO₂, ═O, ═S, ═N(R²²), —N₃, —CN, C₃₋₁₀ carbocycle, and 3- to 10-membered heterocycle,
         wherein the C₃₋₁₀ carbocycle and 3- to 10-membered heterocycle each are optionally substituted with one or more substituents independently selected from halogen, C₁₋₆ alkyl.
   C₁₋₆ haloalkyl, —OR²², —SR²², and —N(R); and C₃₋₁₀ carbocycle and 3- to 10-membered heterocycle, any of which is optionally substituted with one or more substituents independently selected from:
      halogen, C₁₋₆ alkyl, C₁₋₆ haloalkyl, —OR²², —SR²², —N(R²²)₂, —C(O)R²², —C(O)OR²², —OC(O)R²², —OC(O)N(R²²), —C(O)N(R²²)₂, —N(R²²)C(O)R²¹, —N(R²²)C(O)OR²², —N(R²²)C(O)N(R²²)₂, —N(R²²)C(S)N(R²²)₂, —N(R²²)S(O)₂(R²²), —S(O)R²², —S(O)₂R²², —S(O)₂N(R²²)₂, —NO₂, ═O, ═S, ═N(R²²), —N₃, —CN, C₃₋₆ carbocycle, and 3- to 6-membered heterocycle;
         wherein the C₃₋₆ carbocycle and 3- to 6-membered heterocycle are each optionally substituted with one or more substituents independently selected from halogen, C₁₋₄ alkyl, and C₁₋₄ haloalkyl:
R²¹ and R²² are each independently selected at each occurrence from:
   hydrogen;
   C₁₋₄ alkyl optionally substituted with one or more substituents independently selected from halogen, hydroxyl, C₃₋₆ carbocycle, and 3- to 6-membered heterocycle, wherein each C₆ carbocycle and 3- to 6-membered heterocycle is optionally substituted with one or more substituents independently selected from C₁₋₄ alkyl, —N(R²³)₂, and —C(O)N(R²³)₂; and
   C₃₋₆ carbocycle and 3- to 12-membered heterocycle, any of which is optionally substituted with one or more substituents independently selected from halogen, C₁₋₄ alkyl, C₁₋₄ haloalkyl, C₁₋₄ alkoxy, and ═O; and
R²³ is independently selected at each occurrence from hydrogen and C₁₋₄ alkyl.

In some embodiments, a compound of Formula (IIa) is provided:

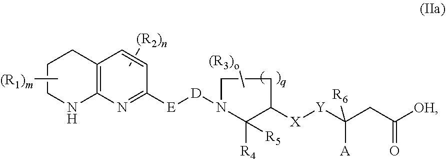

or a pharmaceutically acceptable salt thereof; wherein:
q is 1, 2 or 3;
R¹ and R³ are each independently selected at each occurrence from halogen, C₁₋₄ alkyl, C₁₋₄ haloalkyl, —OR¹¹, —SR¹¹, —N(R¹¹)₂, —C(O)N(R¹¹)₂, —C(O)OR¹¹, ═O, ═S, and —CN;
m is selected from 0, 1, 2, 3, 4, 5, and 6;
o is selected from 0, 1, 2, 3, 4, 5, 6, 7, and 8;
R² is independently selected at each occurrence from halogen, C₁₋₄ alkyl, C₁₋₄ haloalkyl, —OR¹², —SR¹², —N(R¹²)₂, —CN, and —NO₂;
n is 0, 1 or 2;
R⁴ and R⁵ are each independently selected from hydrogen, halogen, C₁₋₄ alkyl, C₁₋₄ haloalkyl, —OR¹³, —SR¹³, —N(R¹³)₂, and —CN; or R⁴ and R⁵ are taken together to form a double bonded substituent selected from ═O, ═S and ═N(R¹³);
D is selected from a bond, —C(O)—, —C≡CCH₂—, and —CH═CHCH₂—;
E is selected from C₁₋₄ alkylene and —(CH₂)Z—,
   wherein Z is selected from —NH—, —S—, —SO₂—, and —O—;
X—Y is selected from: ^{λ}—C(O)N(R¹⁴)—, ^{λ}—N(R¹⁴)C(O)—, ^{λ}—N(R¹⁴)C(O)C(R¹⁵)₂—, ^{λ}—C(O)O—, ^{λ}—C(R¹⁵)₂C(R¹⁵)₂—, ^{λ}—CH═CH—, ^{λ}—C≡C—, ^{λ}—N(R¹⁴)C(R¹⁵)₂—, ^{λ}—C(R¹⁵)₂N(R¹⁴)—, ^{λ}—O—, ^{λ}—OC(R¹⁵)₂—, ^{λ}—C(R¹⁵)₂O—, ^{λ}—SO₂N(R¹⁴)—, and ^{λ}—N(R¹⁴)SO₂—;
   wherein ^{λ} denotes the attachment of X—Y to

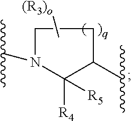

R⁶ and R⁷ are each independently selected at each occurrence from:
   hydrogen, halogen, C₁₋₄ alkyl, C₁₋₄haloalkyl, —OR¹⁶, and —CN;
A is selected from
   —OR¹⁷, —SR¹⁷, —N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R⁷), —C(O)R¹⁷, —C(O)OR¹⁷, —OC(O)R¹⁷, —OC(O)N(R¹⁷)₂, —C(O)N(R¹⁷)₂, —S(O)R¹⁷, —S(O)₂R¹⁷, and —S(O)₂N(R¹⁷)₂;
   C₁₋₆ alkyl optionally substituted with one or more substituents independently selected from:
      halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁸)₂, —N(R¹⁷)C(O)R⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═N(R¹⁷), —N₃, and —CN, C₃₋₁₀ carbocycle, and 3- to 10-membered heterocycle,
         wherein the C₃₋₁₀ carbocycle and 3- to 10-membered heterocycle are each optionally substituted with one or more substituents independently selected from halogen, C₁₋₆ alkyl, C₁₋₆ haloalkyl, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═N(R¹⁷), —N₃, and —CN; and
   C₃₋₁₂ carbocycle and 3- to 12-membered heterocycle, any of which is optionally substituted with one or more substituents independently selected from:
      halogen, —OR¹⁷, —SR¹⁷, —N(R¹⁷)₂, —C(O)R¹⁷, —C(O)OR¹⁷, —OC(O)R¹⁸, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═N(R¹⁷), —N₃, and —CN;
      C₁₋₆ alkyl optionally substituted with one or more substituents independently selected from halogen, —OR¹⁸, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R)C(S)N(R¹⁷)₂, —N(R¹⁷)S(O)₂(R¹⁷), —S(O)R¹⁷, —S(O)₂R′, —S(O)₂N(R¹⁷)₂, —NO₂, ═O, ═S, ═N(R¹⁷), —N₃, and —CN; and
      C₃₋₁₀ carbocycle and 3- to 10-membered heterocycle, any of which is optionally substituted with one or more substituents independently selected from halogen, C₁₋₄alkyl, C₁₋₄ haloalkyl, and ═O;
R¹¹, R¹², R¹³, R¹⁴, and R¹⁶ are each independently selected at each occurrence from hydrogen, C₁₋₄ alkyl, and C₁₋₄ haloalkyl;
R¹⁵ is independently selected at each occurrence from hydrogen, halogen, C₁₋₄ alkyl, and C₁₋₄ haloalkyl;
R¹⁷ is independently selected at each occurrence from:
   hydrogen;
   C₁₋₆ alkyl optionally substituted with one or more substituents independently selected from: halogen, —OR²¹, —SR¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —NO₂, ═O, ═S, ═N(R²), —N₃, and —CN; and
   C₃₋₆ carbocycle and 3- to 6-membered heterocycle, any of which is optionally substituted with one or more substituents independently selected from: halogen, C₁₋₄ alkyl, C₁₋₄ haloalkyl, —OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —N(R²¹)C(O)OR²¹, —N(R²¹)C(O)N(R²¹)₂, —N(R²¹)C(S)N(R²¹)₂, —N(R²¹)S(O)₂(R²¹), —S(O)R²¹, —S(O)₂R²¹, —S(O)₂N(R²¹)₂, —NO₂, ═O, ═S, ═N(R²¹), —N₃, and —CN;
R²¹ is independently selected at each occurrence from:
   hydrogen;
   C₁₋₄ alkyl optionally substituted with one or more substituents independently selected from halogen, hydroxyl, C₃₋₆ carbocycle, and 3- to 6-membered heterocycle, wherein each C₃₋₆ carbocycle and 3- to 6-membered heterocycle is optionally substituted with one or more substituents independently selected from C₁₋₄ alkyl, —N(R²)₂, and —C(O)N(R²³)₂; and
   C₃₋₆ carbocycle and 3- to 12-membered heterocycle, any of which is optionally substituted with one or more substituents independently selected from halogen, C₁₋₄ alkyl, C₁₋₄ haloalkyl, C₁₋₄alkoxy, and ═O; and
R⁷³ is independently selected at each occurrence from hydrogen and C₁₋₄ alkyl.

In other embodiments, a compound of Formula (IIIa) is provided:

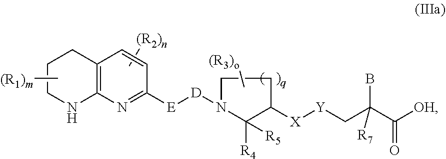

or a pharmaceutically acceptable salt thereof; wherein:
q is 1, 2 or 3;
R¹ and R³ are each independently selected at each occurrence from halogen, C₁₋₄ alkyl, C₁₋₄ haloalkyl, —OR¹¹, —SR¹¹, —N(R¹¹)₂, —C(O)N(R¹¹)₂, —C(O)OR¹¹, ═O, ═S, and —CN;
m is selected from 0, 1, 2, 3, 4, 5, and 6;
o is selected from 0, 1, 2, 3, 4, 5, 6, 7, and 8;
R² is independently selected at each occurrence from halogen, C₁₋₄ alkyl, C₁₋₄ haloalkyl, —OR¹², —SR¹², —N(R¹²)₂, —CN, and —NO₂;
n is 0, 1 or 2;
R⁴ and R⁵ are each independently selected from hydrogen, halogen, C₁₋₄ alkyl, C₁₋₄ haloalkyl, —OR¹³, —SR¹³, —N(R¹³)₂, and —CN; or R⁴ and R⁵ are taken together to form a double bonded substituent selected from ═O, ═S and ═N(R¹³);
D is selected from a bond, —C(O)—, —C≡CCH₂—, and —CH═CHCH₂—;
E is selected from C₁₋₄ alkylene and —(CH₂)Z—,
   wherein Z is selected from —NH—, —S—, —SO—, and —O—;
X—Y is selected from: ^{λ}—C(O)N(R¹⁴)—, ^{λ}—N(R¹⁴)C(O)—, ^{λ}—N(R¹⁴)C(O)C(R¹⁵)₂—, ^{λ}—C(O)O—, ^{λ}—C(R¹⁵)₂C(R¹⁵)₂—, ^{λ}—CH═CH—, ^{λ}—C≡C—, ^{λ}—N(R¹⁵)C(R¹⁵)₂—, ^{λ}—C(R¹⁵)₂N(R¹⁴)—, ^{λ}—O—, ^{λ}—OC(R¹⁵)₂—, ^{λ}—C(R¹⁵)₂O—, ^{λ}—SO₂N(R¹⁴)—, and ^{λ}—N(R¹⁴)SO₂—;

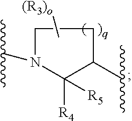

wherein ^{λ} denotes the attachment of X—Y to R₄
R⁶ and R⁷ are each independently selected at each occurrence from:
   hydrogen, halogen, C₁₋₄ alkyl, C₁₋₄haloalkyl, —OR¹⁶, and —CN;
B is selected from:
   —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, and —S(O)₂N(R¹⁸)₂;
   C₁₋₆ alkyl optionally substituted with one or more substituents independently selected from:
      halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═N(R¹⁸), —N₃, —CN, C₃₋₁₀ carbocycle and 3- to 10-membered heterocycle,
         wherein the C₃₋₁₀ carbocycle and 3- to 10-membered heterocycle are optionally substituted with one or more substituents independently selected from halogen, C₁₋₆ alkyl, C₁₋₆ haloalkyl, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R's)C(O)N(R¹⁸)₂—N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═N(R¹⁸), —N₃, and —CN; and
   C₃₋₁₂ carbocycle and 3- to 12-membered heterocycle, any of which is optionally substituted with one or more substituents independently selected from: C₃₋₆ carbocycle and 3- to 6-membered heterocycle, any of which is optionally substituted with one or more substituents independently selected from: halogen, C₁₋₄ alkyl, C₁₋₄ haloalkyl, —OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —N(R²¹)C(O)OR²¹, —N(R²¹)C(O)N(R²¹)₂, —N(R²¹)C(S)N(R²¹)₂, —N(R²¹)S(O)₂(R²¹), —S(O)R²¹, —S(O)₂R²¹, —S(O)₂N(R²¹)₂, —NO₂, ═O, ═S, ═N(R²¹), —N₃, and —CN;
R¹¹, R¹², R¹³, R¹⁴, and R¹⁶ are each independently selected at each occurrence from hydrogen, C₁₋₄ alkyl, and C₁₋₄ haloalkyl;
R¹⁵ is independently selected at each occurrence from hydrogen, halogen, C₁₋₄ alkyl, and C₁₋₄ haloalkyl;
R¹⁸ is independently selected at each occurrence from:
   hydrogen;
   C₁₋₆ alkyl optionally substituted with one or more substituents independently selected from:
      halogen, —OR²², —SR²², —N(R²²)₂, —C(O)R²², —C(O)OR²², —OC(O)R²², —OC(O)N(R²²)₂, —C(O)N(R²²)₂, —N(R²²)C(O)R²², —NO₂, ═O, ═S, ═N(R²²), —N₃, —CN, C₃₋₁₀ carbocycle, and 3- to 10-membered heterocycle,
         wherein the C₃₋₁₀ carbocycle and 3- to 10-membered heterocycle each are optionally substituted with one or more substituents independently selected from halogen, C₁₋₆ alkyl, C₁₋₆ haloalkyl, —OR²², —SR²², and —N(R²²)₂; and
   C₃₋₁₀ carbocycle and 3- to 10-membered heterocycle, any of which is optionally substituted with one or more substituents independently selected from:
      halogen, C₁₋₆ alkyl, C₁₋₆ haloalkyl, —OR²², —SR²², —N(R²²)₂, —C(O)R²², —C(O)OR²², —OC(O)R²², —OC(O)N(R²²)₂, —C(O)N(R²²)₂, —N(R²²)C(O)R²², —N(R²²)C(O)OR²², —N(R²²)C(O)N(R²²)₂, —N(R²²)C(S)N(R²²)₂, —N(R²²)S(O)₂(R²¹), —S(O)R²², —S(O)₂R²², —S(O)₂N(R²²)₂, —NO₂, ═O, ═S, ═N(R²²), —N₃, —CN, C₃₋₆ carbocycle, and 3- to 6-membered heterocycle;
         wherein the C₃₋₆ carbocycle and 3- to 6-membered heterocycle are each optionally substituted with one or more substituents independently selected from halogen, C₁₋₄ alkyl, and C₁₋₄ haloalkyl;
R²² is independently selected at each occurrence from:
   hydrogen;
   C₁₋₄ alkyl optionally substituted with one or more substituents independently selected from halogen, hydroxyl, C₃₋₆ carbocycle, and 3- to 6-membered heterocycle, wherein each C₆ carbocycle and 3- to 6-membered heterocycle is optionally substituted with one or more substituents independently selected from C₁₋₄ alkyl, —N(R²)₂, and —C(O)N(R²¹)₂; and
   C₃₋₆ carbocycle and 3- to 12-membered heterocycle, any of which is optionally substituted with one or more substituents independently selected from halogen, C₁₋₄ alkyl, C₁₋₄haloalkyl, C₁₋₄alkoxy, and ═O; and
R²³ is independently selected at each occurrence from hydrogen and C₁₋₄ alkyl.

In some embodiments, for the compound or salt of Formulas (Ia), (IIa) or (IIIa), q is 1, 2, or 3. In some embodiments, q is selected from 1, 2 and 3. In some embodiments, q is selected from 1 and 2. In some embodiments, q is selected from 2 and 3. In some embodiments. In some embodiments, q is 1. In some embodiments, q is 2. In some embodiments, q is 3.

In some embodiments, for the compound or salt of Formulas (Ia), (IIa) or (IIIa), R¹ and R³ are each independently selected at each occurrence from halogen, C₁₋₄ alkyl, C₁₋₄ haloalkyl, —OR¹¹, —N(R¹¹)₂, —C(O)N(R¹¹)₂, —C(O)OR¹¹, ═O, and —CN. In some embodiments, R¹ and R³ are each independently selected at each occurrence from halogen, C₁₋₄ alkyl, C₁₋₄haloalkyl, —OR¹¹, —N(R¹¹)₂, —C(O)N(R¹¹)₂, —C(O)OR¹¹, and ═O. In some embodiments, R¹ and R³ are each independently selected at each occurrence from halogen, C₁₋₄alkyl, C₁₋₄haloalkyl, —OR¹¹, —N(R¹¹)₂, —C(O)N(R¹¹)₂, —C(O)OR¹¹, and —CN. In some embodiments, R¹ and R³ are each independently selected at each occurrence from halogen, C₁₋₄ alkyl, C₁₋₄haloalkyl, —OR¹¹, —N(R¹¹)₂, —C(O)N(R¹¹)₂, and —C(O)OR¹¹. In some embodiments, R¹ and R³ are each independently selected at each occurrence from halogen, C₁₋₄alkyl, C₁₋₄ haloalkyl, —OR¹¹, —C(O)N(R¹¹)₂, and —C(O)OR¹¹. In some embodiments, R¹ and R³ are each independently selected at each occurrence from halogen, C₁₋₄ alkyl, C₁₋₄ haloalkyl, —C(O)N(R¹¹)₂, and —C(O)OR¹¹. In some embodiments. R¹ and R³ are each independently selected at each occurrence from halogen, C₁₋₄ alkyl, C₁₋₄haloalkyl, and —C(O)N(R¹¹)₂. In some embodiments, R¹ and R³ are each independently selected at each occurrence from halogen, C₁₋₄alkyl, —C(O)N(R¹¹)₂, and —C(O)OR¹¹. In some embodiments, R¹ and R³ are each independently selected at each occurrence from halogen, C₁₋₄ alkyl, and —C(O)N(R¹¹)₂.

In some embodiments, for the compound or salt of Formulas (Ia), (IIa) or (IIIa), m is selected from 0, 1, 2, 3, 4, 5, and 6. In some embodiments, m is selected from 0, 1, 2, 3, 4, and 5. In some embodiments, m is selected from 0, 1, 2, 3, and 4. In some embodiments, m is selected from 1, 2, 3, and 4. In some embodiments, m is selected from 0, 1, 2, and 3. In some embodiments, m is selected from 0, 1, and 2. In some embodiments, m is selected from 0, and 1. In some embodiments, m is 0. In some embodiments, m is 1. In some embodiments, m is 2. In some embodiments, m is 3. In some embodiments, m is 4. In some embodiments, m is 5. In some embodiments, m is 6.

In some embodiments, for the compound or salt of Formulas (Ia), (IIa) or (IIIa), o is selected from 0, 1, 2, 3, 4, 5, 6, 7, and 8. In some embodiments, o is selected from 0, 1, 2, 3, 4, 5, 6, and 7. In some embodiments, o is selected from 0, 1, 2, 3, 4, 5, and 6. In some embodiments, o is selected from 0, 1, 2, 3, 4, and 5. In some embodiments, o is selected from 0, 1, 2, 3, and 4. In some embodiments, o is selected from 1, 2, 3, and 4. In some embodiments, o is selected from 0, 1, 2, and 3. In some embodiments, o is selected from 0, 1, and 2. In some embodiments, o is selected from 0, and 1. In some embodiments, o is 0. In some embodiments, o is 1. In some embodiments, o is 2. In some embodiments, o is 3. In some embodiments, o is 4. In some embodiments, o is 5. In some embodiments, o is 6. In some embodiments, o is 7. In some embodiments, o is 8.

In some embodiments, for the compound or salt of Formulas (Ia), (IIa) or (IIIa), R² is independently selected at each occurrence from halogen, C₁₋₄ alkyl, C₁₋₄ haloalkyl, —OR¹², —SR¹², —N(R¹²)₂, and —CN. In some embodiments, R² is independently selected at each occurrence from halogen, C₁₋₄alkyl, C₁₋₄haloalkyl, —OR¹², —N(R¹²)₂, and —CN. In some embodiments, R² is independently selected at each occurrence from halogen, C₁₋₄ alkyl, C₁₋₄ haloalkyl, —OR¹², and —CN. In some embodiments, R² is independently selected at each occurrence from halogen, C₁₋₄ alkyl, C₁₋₄haloalkyl, —N(R¹²)₂, and —CN. In some embodiments, R² is independently selected at each occurrence from halogen, C₁₋₄ alkyl, C₁₋₄ haloalkyl, —OR¹², and —N(R¹²)₂. In some embodiments, R² is independently selected at each occurrence from halogen, C₁₋₄ alkyl, C₁₋₄ haloalkyl, and —CN. In some embodiments, R² is independently selected at each occurrence from halogen, C₁₋₄ alkyl, C₁₋₄ haloalkyl, and —N(R¹²)₂. In some embodiments, R² is independently selected at each occurrence from halogen, C₁₋₄ alkyl, C₁₋₄ haloalkyl, and —OR¹². In some embodiments, R² is independently selected at each occurrence from halogen, C₁₋₄ alkyl, and C₁₋₄ haloalkyl. In some embodiments, R² is independently selected at each occurrence from halogen. In some embodiments. R² is independently selected at each occurrence from C₁₋₄ alkyl. In some embodiments, R² is independently selected at each occurrence from C₁₋₄ haloalkyl.

In some embodiments, for the compound or salt of Formulas (Ia), (IIa) or (IIIa), n is 0, 1 or 2. In some embodiments, n is selected from 0, 1, and 2. In some embodiments, n is selected from 0, and 1. In some embodiments, n is selected from 1, and 2. In some embodiments, n is 0. In some embodiments, n is 1. In some embodiments, n is 2.

In some embodiments, for the compound or salt of Formulas (Ia), (IIa) or (IIIa), R⁴ and R¹ are each independently selected from hydrogen, halogen, C₁₋₄ alkyl, C₁₋₄haloalkyl, —OR¹¹, —SR¹¹, —N(R¹³)₂, and —CN; or R⁴ and R⁵ are taken together to form a double bonded substituent selected from ═O, ═S and ═N(R¹³). In some embodiments, R⁴ and R⁵ are each independently selected from hydrogen, halogen, C₁₋₄alkyl, C₁₋₄ haloalkyl, —OR¹³, —N(R¹³)₂, and —CN; or R⁴ and R⁵ are taken together to form a double bonded substituent selected from ═O, and ═N(R¹³). In some embodiments, R⁴ and R⁵ are each independently selected from hydrogen, halogen, C₁₋₄ alkyl, C₁₋₄ haloalkyl, —OR¹³, —N(R¹³)₂, and —CN; or R⁴ and R⁵ are taken together to form ═O. In some embodiments, R⁴ and R⁵ are each independently selected from hydrogen, halogen, C₁₋₄ alkyl, C₁₋₄ haloalkyl, —OR¹³, and —N(R¹³)₂; or R⁴ and R⁵ are taken together to form a double bonded substituent selected from ═O, and ═N(R¹³). In some embodiments, R⁴ and R⁵ are each independently selected from hydrogen, halogen, C₁₋₄ alkyl, C₁₋₄ haloalkyl, —OR¹³, and —N(R¹³)₂; or R⁴ and R⁵ are taken together to form ═O. In some embodiments, R⁴ and R⁵ are each independently selected from hydrogen, halogen, C₁₋₄ alkyl, C₁₋₄ haloalkyl, and —OR¹³; or R⁴ and R⁵ are taken together to form ═O. In some embodiments, R⁴ and R⁵ are each independently selected from hydrogen, halogen, C₁₋₄ alkyl, C₁₋₄ haloalkyl, and —N(R¹³)₂; or R⁴ and R⁵ are taken together to form ═O. In some embodiments. R⁴ and R⁵ are each independently selected from hydrogen, halogen, C₁₋₄ alkyl, and C₁₋₄ haloalkyl; or R⁴ and R⁵ are taken together to form ═O. In some embodiments, R⁴ and R⁵ are each independently selected from hydrogen, halogen, and C₁₋₄ alkyl; or R⁴ and R⁵ are taken together to form ═O.

In some embodiments, for the compound or salt of Formulas (Ia), (IIa) or (IIIa), R⁴ and R⁵ are each independently selected from hydrogen, halogen, C₁₋₄ alkyl, C₁₋₄ haloalkyl, —OR¹³, —SR¹³, —N(R¹³)₂, and —CN. In some embodiments, R⁴ and R⁵ are each independently selected from hydrogen, halogen, C₁₋₄alkyl, C₁₋₄ haloalkyl, —OR¹³, —N(R¹³)₂, and —CN. In some embodiments, R⁴ and R⁵ are each independently selected from hydrogen, halogen, C₁₋₄ alkyl, C₁₋₄ haloalkyl, —OR¹³, and —N(R³)₂. In some embodiments. R⁴ and R⁵ are each independently selected from hydrogen, halogen, C₁₋₄ alkyl, C₁₋₄ haloalkyl, —OR¹³, and —CN. In some embodiments. R⁴ and R⁵ are each independently selected from hydrogen, halogen, C₁₋₄ alkyl, C₁₋₄ haloalkyl, —N(R¹³)₂ and —CN. In some embodiments, R⁴ and R⁵ are each independently selected from hydrogen, halogen, C₁₋₄ alkyl, C₁₋₄ haloalkyl, and —CN. In some embodiments, R⁴ and R⁵ are each independently selected from hydrogen, halogen, C₁₋₄ alkyl, and C₁₋₄ haloalkyl. In some embodiments, R⁴ and R⁵ are each independently selected from hydrogen, halogen, and C₁₋₄ alkyl. In some embodiments, R⁴ and R⁵ are each independently selected from hydrogen, and halogen. In some embodiments, R⁴ and R⁵ are each hydrogen.

In some embodiments, for the compound or salt of Formulas (Ia), (IIa) or (IIIa), R⁴ and R⁵ are taken together to form a double bonded substituent selected from ═O, ═S and ═N(R¹³). In some embodiments, R⁴ and R⁵ are taken together to form a double bonded substituent selected from ═O, and ═N(R¹³). In some embodiments, for the compound or salt of Formula (I), R⁴ and R⁵ are taken together to form a double bonded substituent selected from ═O and ═S. In some embodiments, R⁴ and R⁵ are taken together to form ═O.

In some embodiments, for the compound or salt of Formulas (Ia), (IIa) or (IIIa), D is selected from a bond, —C(O)—, and —C≡CCH₂—. In some embodiments, D is selected from a bond, —C(O)—, and —CH═CHCH₂—. In some embodiments, D is selected from a bond, and —C(O)—. In some embodiments, D is a bond. In some embodiments, D is —C(O)—.

In some embodiments, for the compound or salt of Formulas (Ia), (IIa) or (IIa), E is selected from C₁₋₄ alkylene. In some embodiments, E is selected from —(CH₂)Z—, wherein Z is selected from —NH—, —S—, —SO₂—, and —O—. In some embodiments, E is selected from C₁₋₄ alkylene and —(CH₂)Z—, wherein Z is selected from —NH—, —S—, and —O—. In some embodiments, E is selected from C₁₋₄ alkylene and —(CH₂)Z—, wherein Z is selected from —NH—, —SO₂—, and —O—. In some embodiments, E is selected from C₁₋₄ alkylene and —(CH₂)Z—, wherein Z is selected from —NH— and —O—.

In some embodiments, for the compound or salt of Formulas (Ia), (IIa) or (IIIa), D is selected from a bond and —C(O)—; and E is selected from C₁₋₄ alkylene. In some embodiments, D is a bond; and E is selected from C₁₋₄ alkylene. In some embodiments, D is —C(O)—; and E is selected from C₁₋₄alkylene.

In some embodiments, for the compound or salt of Formulas (Ia), (IIa) or (IIIa), X—Y is selected from:
^{λ}—C(O)N(R¹⁴)—, ^{λ}—N(R¹⁴)C(O)—, ^{λ}—N(R¹⁴)C(O)C(R¹⁵)₂—, ^{λ}—C(O)O—, ^{λ}—C(R¹⁵)₂C(R¹⁵)₂—, ^{λ}—N(R¹⁴)C(R¹⁵)₂N(R¹⁴)—, ^{λ}—C(R¹⁵)₂N(R¹⁴)—, ^{λ}—O—, ^{λ}—OC(R¹⁵)₂—, ^{λ}—C(R¹⁵)₂O—, ^{λ}—SO₂N(R¹⁴)—, and ^{λ}—N(R¹⁴)SO₂—; wherein λ denotes the attachment of X—Y to

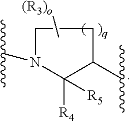

In some embodiments, X—Y is selected from: ^{λ}—C(O)N(R¹⁴)—, ^{λ}—N(R¹⁴)C(O)—, ^{λ}—N(R¹⁴)C(O)C(R¹⁵)₂—, ^{λ}—C(O)O—, ^{λ}—C(R¹⁵)₂C(R¹⁵)₂—, ^{λ}—CH═CH—, ^{λ}—C≡C—, ^{λ}—N(R¹⁴)C(R¹⁵)₂—, —C(R¹⁵)₂N(R¹⁴)—, —O—, ^{λ}—OC(R¹⁵)₂—, and ^{λ}—C(R¹⁵)₂O—. In some embodiments, X—Y is selected from: ^{λ}—C(O)N(R¹⁴)—, ^{λ}—N(R¹⁴)C(O)—, ^{λ}—N(R¹⁴)C(O)C(R¹⁵)₂—, ^{λ}—C(R¹⁵)₂, ^{λ}—C(R¹⁵)₂—, ^{λ}—CH═CH—, ^{λ}—C≡—, ^{λ}—N(R¹⁴)C(R¹⁵)₂—, ^{λ}—C(R¹⁵)₂N(R¹⁴)—, ^{λ}—O—, ^{λ}—OC(R¹⁵)₂—, ^{λ}—C(R¹⁵)₂O—, ^{λ}—SO₂N(R¹⁴)—, and ^{λ}—N(R¹⁴)SO₂. In some embodiments, X—Y is selected from: ^{λ}—C(O)N(R¹⁴)—, ^{λ}—N(R¹⁴)C(O)—, ^{λ}—N(R¹⁴)C(O)C(R¹⁵)₂—, ^{λ}—C(O)O—, ^{λ}—C(R¹⁵)₂C(R¹⁵)₂—, ^{λ}—N(R¹⁴)C(R¹⁵)₂—, ^{λ}—C(R¹⁵)₂N(R¹⁴)—, ^{λ}—O—, ^{λ}—OC(R¹⁵)₂—, and ^{λ}—C(R¹⁵)₂O—. In some embodiments, X—Y is selected from: ^{λ}—C(O)N(R¹⁴)—, ^{λ}—N(R¹⁴)C(O)—, ^{λ}—N(R¹⁴)C(O)C(R¹⁵)₂—, ^{λ}—C(R¹⁵)₂C(R¹⁵)₂—, ^{λ}—N(R¹⁴)C(R¹⁵)₂—, ^{λ}—C(R¹⁵)₂N(R¹⁴)—, ^{λ}—O—, ^{λ}—OC(R¹⁵)₂—, ^{λ}—C(R¹⁵)₂O—, ^{λ}—SO₂N(R¹⁴)—, and ^{λ}—N(R¹⁴)SO₂—. In some embodiments, X—Y is selected from: ^{λ}—C(O)N(R¹⁴)—, ^{λ}—N(R¹⁴)C(O)—, ^{λ}—N(R¹⁴)C(O)C(R¹⁵)₂—, ^{λ}—C(R¹⁵)₂C(R¹⁵)₂—, ^{λ}—CH═CH—, ^{λ}—C≡C—, ^{λ}—N(R¹⁴)C(R¹⁵)₂—, ^{λ}—C(R¹⁵)₂N(R¹⁴)—, ^{λ}—O—, ^{λ}—OC(R¹⁵)₂—, and ^{λ}—C(R¹⁵)₂O—.

In some embodiments, for the compound or salt of Formulas (Ia), (IIa) or (IIIa), X—Y is selected from: ^{λ}—C(O)N(R¹⁴)—, ^{λ}—N(R¹⁴)C(O)—, ^{λ}—C(R¹⁵)₂C(R¹⁵)₂—, ^{λ}—N(R¹⁴)C(R¹⁵)₂—, ^{λ}—C(R¹⁵)₂N(R¹⁴)—, ^{λ}—O—, ^{λ}—OC(R¹⁵)₂—, and ^{λ}—C(R¹⁵)₂O—. In some embodiments, X—Y is selected from: ^{λ}—C(O)N(R¹⁴)—, ^{λ}—N(R¹⁴)C(O)—, ^{λ}—N(R¹⁴)C(R¹⁵)₂—, ^{λ}—C(R¹⁵)₂N(R¹⁴)—, ^{λ}—O—, ^{λ}—OC(R¹⁵)₂—, and ^{λ}—C(R¹⁵)₂O—. In some embodiments, X—Y is selected from: ^{λ}—C(O)N(R¹⁴)—, ^{λ}—N(R¹⁴)C(O)—, ^{λ}—N(R¹⁴)C(R¹⁵)₂—, ^{λ}—C(R¹⁵)₂N(R¹⁴)—, ^{λ}—OC(R¹⁵)₂—, and ^{λ}—C(R¹⁵)₂O—. In some embodiments, X—Y is selected from: ^{λ}—C(O)N(R¹⁴)—, ^{λ}—N(R¹⁴)C(O)—, ^{λ}—OC(R¹⁵)₂—, and ^{λ}—C(R¹⁵)₂O—. In some embodiments, X—Y is selected from: ^{λ}—C(O)N(R¹⁴)—, ^{λ}—N(R¹⁴)C(O)—, ^{λ}—OC(R¹⁵)₂—, and ^{λ}—C(R¹⁵)₂O—. In some embodiments, X—Y is selected from: ^{λ}—C(O)N(R¹⁴)—, and ^{λ}—N(R¹⁴)C(O)—. In some embodiments, X—Y is selected from: ^{λ}—OC(R¹⁵)₂—, and ^{λ}—C(R¹⁵)₂O—. In some embodiments, X—Y is ^{λ}—C(O)N(R¹⁴)—. In some embodiments. X—Y is ^{λ}—N(R¹⁴)C(O)—. In some embodiments. X—Y is ^{λ}—C(R¹⁵)₂C(R¹⁵)₂—. In some embodiments, X—Y is ^{λ}—N(R¹⁴)C(R¹⁵)₂—. In some embodiments, X—Y is ^{λ}—C(R¹⁵)₂N(R¹⁴)—. In some embodiments. X—Y is ^{λ}—O—. In some embodiments. X—Y is ^{λ}—OC(R¹⁵)₂—. In some embodiments, X—Y is ^{λ}—C(R¹⁵)₂O—.

In some embodiments, for the compound or salt of Formulas (Ia), (IIa) or (IIIa), X—Y is selected from ^{λ}—C(O)N(R¹⁴)—, ^{λ}—N(R¹⁴)C(O)—, ^{λ}—N(R¹⁴)C(O)CH₂—, ^{λ}—CH₂CH₂—, ^{λ}—N(R¹⁴)CH₂—, ^{λ}—CH₂N(R¹⁴)—, ^{λ}—O—, ^{λ}—OCH₂—, and ^{λ}—CH₂O—; and R¹⁴ is selected at each occurrence from hydrogen and C₁₋₄alkyl. In some embodiments, X—Y is selected from: ^{λ}—C(O)N(R¹⁴)—, ^{λ}—N(R¹⁴)C(O)—, ^{λ}—N(R¹⁴)C(R¹⁵)₂—, ^{λ}—C(R¹⁵)₂N(R¹⁴)—, ^{λ}—OC(R¹⁵)₂—, and ^{λ}—C(R¹⁵)₂O—; and R¹⁴ is selected at each occurrence from hydrogen and C₁₋₄ alkyl. In some embodiments, X—Y is selected from: ^{λ}—C(O)N(R¹⁴)—, ^{λ}—N(R¹⁵)C(O)—, —OC(R¹⁵)₂—, and ^{λ}—C(R¹⁵)₂O—; and R¹⁴ is selected at each occurrence from hydrogen and C₁₋₄ alkyl. In some embodiments. X—Y is selected from: ^{λ}—C(O)N(R¹⁴)—, ^{λ}—N(R¹⁴)C(O)—, ^{λ}—OC(R¹⁵)₂—, and ^{λ}—C(R¹⁵)₂O—; and R¹⁴ is selected at each occurrence from hydrogen and C₁₋₄ alkyl. In some embodiments, X—Y is selected from: ^{λ}—C(O)N(R¹⁴)—, and ^{λ}—N(R¹⁴)C(O)—; and R¹⁴ is selected at each occurrence from hydrogen and C₁₋₄ alkyl.

In some embodiments, for the compound or salt of Formulas (Ia), (IIa) or (IIIa), X—Y is selected from: ^{λ}—C(O)N(H)—, ^{λ}—N(H)C(O)—, ^{λ}—CH₂CH₂—, ^{λ}—N(H)CH₂—, ^{λ}—CH₂N(H)—, —O—, ^{λ}—OCH₂—, and ^{λ}—CH₂O—. In some embodiments. X—Y is selected from: ^{λ}—C(O)N(H)—, ^{λ}—N(H)C(O)—, ^{λ}—N(H)CH₂—, ^{λ}—CH₂N(H)—, ^{λ}—O—, —OCH₂—, and ^{λ}—CH₂O—. In some embodiments, X—Y is selected from: ^{λ}—C(O)N(H)—, ^{λ}—N(H)C(O)—, ^{λ}—N(H)CH₂—, —CH₂N(H)—, ^{λ}—OCH₂, and ^{λ}—CH₂O—. In some embodiments, X—Y is selected from: ^{λ}—C(O)N(H)—, ^{λ}—N(H)C(O)—, ^{λ}—OCH—, and ^{λ}—CH₂O—. In some embodiments, X—Y is selected from: ^{λ}—C(O)N(H)—, ^{λ}—N(H)C(O)—, ^{λ}—OCH₂—, and ^{λ}—CH₂—. In some embodiments, X—Y is selected from: ^{λ}—C(O)N(H)—, and ^{λ}—N(H)C(O)—. In some embodiments, X—Y is selected from: ^{λ}—OCH₂—, and ^{λ}—CH₂O—. In some embodiments, X—Y is ^{λ}—C(O)N(H)—. In some embodiments. X—Y is ^{λ}—N(H)C(O)—. In some embodiments, X—Y is ^{λ}—CH₂CH₂—. In some embodiments, X—Y is ^{λ}—N(H)CH₂—. In some embodiments, X—Y is ^{λ}—CH₂N(H)—. In some embodiments, X—Y is ^{λ}—O—. In some embodiments, X—Y is ^{λ}—OCH₂—. In some embodiments, X—Y is ^{λ}—CH₂O—.

In some embodiments, for the compound or salt of Formulas (Ia), (IIa) or (IIIa), R⁶ and R⁷ are each independently selected at each occurrence from: hydrogen, halogen, C₁₋₄alkyl, C₁₋₄ haloalkyl, —OR¹⁶, —N(R¹⁶)₂, and —CN. In some embodiments, R⁶ and R⁷ are each independently selected at each occurrence from: hydrogen, halogen, C₁₋₄ alkyl, C₁₋₄ haloalkyl, and —OR¹⁶. In some embodiments, R⁶ and R₇ are each independently selected at each occurrence from: hydrogen, halogen, C₁₋₄ alkyl, and —OR¹⁶. In some embodiments, R⁶ and R⁷ are each independently selected at each occurrence from: hydrogen, halogen, and —OR¹⁶. In some embodiments, R⁶ and R⁷ are each independently selected at each occurrence from: hydrogen and halogen. In some embodiments, R⁶ and R₇ are each independently selected at each occurrence from: hydrogen and —OR¹⁶. In some embodiments, R⁶ and R⁷ are each hydrogen.

In some embodiments, for the compound or salt of Formulas (Ia), (IIa) or (IIIa), A is selected from (i) and (ii):
(i) hydrogen, halogen, and —CN, or A and R⁶ come together to form a C₃₋₆ carbocycle or 3- to 6-membered heterocycle; and
(ii) C₃₋₁₂ carbocycle and 3- to 12-membered heterocycle, any of which is optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═N(R¹⁷), —N₃, and —CN;
   C₁₋₆ alkyl optionally substituted with one or more substituents independently selected from halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═N(R¹⁷), —N₃, and —CN; and
   C₃₋₁₀ carbocycle and 3- to 10-membered heterocycle, any of which is optionally substituted with one or more substituents independently selected from halogen. C₁₋₄alkyl, C₁₋₄ haloalkyl, and ═O.

In some embodiments, for the compound or salt of Formulas (Ia), (IIa) or (IIIa), A is selected from (i) and (ii):
(i) hydrogen; and
(ii) C₃₋₁₂ carbocycle and 3- to 12-membered heterocycle, any of which is optionally substituted with one or more substituents independently selected from:
   halogen, —OR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹¹, —S(O)₂R¹¹, —S(O)₂N(R¹⁷)₂, —NO₂, ═O, and —CN;
   C₁₋₆ alkyl optionally substituted with one or more substituents independently selected from halogen, —OR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, —CN; and
   C₃₋₁₀ carbocycle and 3- to 10-membered heterocycle, any of which is optionally substituted with one or more substituents independently selected from halogen, C₁₋₄ alkyl, C₁₋₄ haloalkyl, and ═O.

In some embodiments, for the compound or salt of Formulas (Ia), (IIa) or (IIIa), A is selected from (i) and (ii):
(i) hydrogen; and
(ii) C₃₋₁₀ carbocycle and 3- to 12-membered heterocycle, any of which is optionally substituted with one or more substituents independently selected from:
   halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, —NO₂, ═O, and —CN;
   C₁₋₆ alkyl optionally substituted with one or more substituents independently selected from halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —N(R¹⁷)S(O)₂(R¹⁷), —S(O)₂R¹⁷, —S(O)₂N(R¹⁷)₂, —NO₂, ═O, —CN; and
   C₃₋₁₀ carbocycle and 3- to 10-membered heterocycle, any of which is optionally substituted with one or more substituents independently selected from halogen, C₁₋₄ alkyl, C₁₋₄haloalkyl, and ═O.

In some embodiments, for the compound or salt of Formulas (Ia), (IIa) or (IIIa), A is selected from (i) and (ii):
(i) hydrogen; and
(ii) C₃₋₁₂ carbocycle and 3- to 12-membered heterocycle, any of which is optionally substituted with one or more substituents independently selected from:
   halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —NO₂, ═O, and —CN;
   C₁₋₆ alkyl optionally substituted with one or more substituents independently selected from halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —NO₂, ═O, —CN; and
   C₃₋₁₀ carbocycle and 3- to 10-membered heterocycle, any of which is optionally substituted with one or more substituents independently selected from halogen, C₁₋₄ alkyl, C₁₋₄ haloalkyl, and ═O.

In some embodiments, for the compound or salt of Formulas (Ia), (IIa) or (IIIa), A is selected from (i) and (ii):
(i) hydrogen; and
(ii) C₃₋₁₂ carbocycle and 3- to 12-membered heterocycle, any of which is optionally substituted with one or more substituents independently selected from: halogen; —OR¹⁷; C₁₋₆ alkyl optionally substituted with one or more substituents independently selected from halogen; and C₃₋₁₀, carbocycle and 3- to 10-membered heterocycle, any of which is optionally substituted with one or more substituents independently selected from halogen, C₁₋₄ alkyl, C₁₋₄ haloalkyl, and ═O; wherein R¹⁷ is independently selected at each occurrence from hydrogen, C₁₋₆ alkyl, C₃₋₆ carbocycle and 3- to 6-membered heterocycle.

In some embodiments, for the compound or salt of Formulas (Ia), (IIa) or (IIIa), A is selected from hydrogen, halogen, and —CN, or A and R⁶ come together to form a C₃ carbocycle or 3- to 6-membered heterocycle. In some embodiments, A is selected from hydrogen and halogen, or A and R⁶ come together to form a C₃₋₆ carbocycle. In some embodiments, A is hydrogen, or A and R₆ come together to form a C₃₋₆ carbocycle. In some embodiments, A is selected from hydrogen, halogen, and —CN. In some embodiments, A is selected from hydrogen and halogen. In some embodiments, A is selected from hydrogen, and —CN. In some embodiments, A is hydrogen. In some embodiments, A and R⁶ come together to form a C₁₋₆ carbocycle or 3- to 6-membered heterocycle. In some embodiments, A and R⁶ come together to form a C₃₋₆ carbocycle.

In some embodiments, for the compound or salt of Formulas (Ia), (IIa) or (IIIa), A is selected from C₃₋₁₂ carbocycle and 3- to 12-membered heterocycle, any of which is optionally substituted with one or more substituents independently selected from:
halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷), —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═N(R¹⁷), —N₃, and —CN;
C₁₋₆ alkyl optionally substituted with one or more substituents independently selected from halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═N(R¹⁷), —N₃, and —CN; and
C₃₋₁₀ carbocycle and 3- to 10-membered heterocycle, any of which is optionally substituted with one or more substituents independently selected from halogen, C₁₋₄ alkyl, C₁₋₄ haloalkyl, and ═O.

In some embodiments, for the compound or salt of Formulas (Ia), (IIa) or (IIIa), A is selected from C₃₋₁₂ carbocycle and 3- to 12-membered heterocycle, any of which is optionally substituted with one or more substituents independently selected from:
halogen, —OR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, and —CN;
C₁₋₆ alkyl optionally substituted with one or more substituents independently selected from halogen, —OR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, —CN; and
C₃₋₁₀ carbocycle and 3- to 10-membered heterocycle, any of which is optionally substituted with one or more substituents independently selected from halogen, C₁₋₄ alkyl, C₁₋₄ haloalkyl, and ═O.

In some embodiments for the compound or salt of Formulas (Ia), (IIa) or (IIIa), A is selected from C₃₋₁₂ carbocycle and 3- to 12-membered heterocycle, any of which is optionally substituted with one or more substituents independently selected from:
halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —NO₂, ═O, and —CN;
C₁₋₆ alkyl optionally substituted with one or more substituents independently selected from halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —NO₂, ═O, —CN; and
C₃₋₁₀ carbocycle and 3- to 10-membered heterocycle, any of which is optionally substituted with one or more substituents independently selected from halogen, C₁₋₄ alkyl, C₁₋₄ haloalkyl, and ═O.

In some embodiments, for the compound or salt of Formulas (Ia), (IIa) or (IIIa), A is selected from C₃₋₁₂ carbocycle and 3- to 12-membered heterocycle, any of which is optionally substituted with one or more substituents independently selected from: halogen; —OR¹⁷; C₁₋₆ alkyl optionally substituted with one or more substituents independently selected from halogen; and C₃₋₁₀ carbocycle and 3- to 10-membered heterocycle, any of which is optionally substituted with one or more substituents independently selected from halogen, C₁₋₄alkyl, C₁₋₄haloalkyl, and ═O.

In some embodiments, for the compound or salt of Formulas (Ia), (IIa) or (IIIa), R¹⁷ is independently selected at each occurrence from:
hydrogen;
C₁₋₆ alkyl optionally substituted with one or more substituents independently selected from: halogen, —OR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —NO₂, ═O, and —CN; and
C₃₋₆ carbocycle and 3- to 6-membered heterocycle, any of which is optionally substituted with one or more substituents independently selected from: halogen, C is alkyl, C₁₋₄ haloalkyl, —OR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —N(R²¹)C(O)OR²¹, —N(R²¹)C(O)N(R²¹)₂, —N(R²¹)C(S)N(R²¹)₂, —N(R²¹)S(O)₂(R²¹), —S(O)R²¹, —S(O)₂R²¹, —S(O)₂N(R²¹)₂, —NO₂, ═O, and —CN.

In some embodiments, for the compound or salt of Formulas (Ia), (IIa) or (IIIa), R¹⁷ is independently selected at each occurrence from:
hydrogen;
C₁₋₆ alkyl optionally substituted with one or more substituents independently selected from: halogen, —OR²¹, —N(R²¹)₂, —C(O)OR²¹, —OC(O)R²¹, —C(O)N(R²¹)₂, —N(R²¹)C(O)R², ═O, and —CN, and
C₃₋₆ carbocycle and 3- to 6-membered heterocycle, any of which is optionally substituted with one or more substituents independently selected from: halogen, C₁₋₄ alkyl, C₁₋₄ haloalkyl, —OR²¹, —N(R²¹)₂, —C(O)R²¹, —C(O)OR²¹, —OC(O)R²¹, —C(O)N(R²¹)₂, —N(R²¹)C(O)R²¹, —N(R²¹)S(O)₂(R²¹), —S(O)₂R²¹, —S(O)₂N(R²¹)₂, ═O, and —CN.

In some embodiments, for the compound or salt of Formulas (Ia), (IIa) or (IIIa), R¹⁷ is independently selected at each occurrence from hydrogen; C₁₋₆ alkyl optionally substituted with one or more substituents independently selected from: halogen, —OR²¹, —N(R²¹)₂, —C(O)N(R²¹)₂, —N(R²¹)C(O)R²¹, ═O, and —CN; and C₃₋₆ carbocycle and 3- to 6-membered heterocycle, any of which is optionally substituted with one or more substituents independently selected from: halogen, C₁₋₄ alkyl, C₁₋₄ haloalkyl, —OR²¹, —N(R²¹)₂, —C(O)R²¹, —C(O)N(R²¹)₂, —N(R²¹)C(O)R²¹, ═O, and —CN. In some embodiments, R¹⁷ is independently selected at each occurrence from hydrogen, C₁₋₆ alkyl, C₃₋₆ carbocycle and 3- to 6-membered heterocycle.

In some embodiments, for the compound or salt of Formulas (Ia), (IIa) or (IIIa), R²¹ is independently selected at each occurrence from:
hydrogen;
C₁₋₄ alkyl optionally substituted with one or more substituents independently selected from halogen, hydroxyl, C₃₋₆ carbocycle, and 3- to 6-membered heterocycle, wherein each C₃₋₆ carbocycle and 3- to 6-membered heterocycle is optionally substituted with one or more substituents independently selected from halogen C₁₋₄ alkyl, and —C(O)N(R²³)₂; and
C₃₋₆ carbocycle and 3- to 12-membered heterocycle, any of which is optionally substituted with one or more substituents independently selected from halogen, C₁₋₄ alkyl, C₁₋₄haloalkyl, C₁₋₄ alkoxy, and ═O; and
R²³ is independently selected at each occurrence from hydrogen and C₁₋₄ alkyl.

In some embodiments, for the compound or salt of Formulas (Ia), (IIa) or (IIIa), R²¹ is independently selected at each occurrence from hydrogen, C₁₋₄ alkyl, C₃₋₆ carbocycle, and 3- to 6-membered heterocycle, wherein the C₃₋₆ carbocycle and 3- to 6-membered heterocycle are each optionally substituted with one or more substituents independently selected from C₁₋₄alkyl and C₁₋₄ alkoxy; and R²³ is independently selected at each occurrence from hydrogen and C₁₋₄ alkyl.

In some embodiments, for the compound or salt of Formulas (Ia), (IIa) or (IIIa), A is selected from C₃₋₂ carbocycle and 3- to 12-membered heterocycle, any of which is optionally substituted with one or more substituents. In some embodiments, the C₃₋₁₂ carbocycle and 3- to 12-membered heterocycle of A is selected from phenyl; pyridine; indane; chromane; benzodioxole; 2,3-dihydrobenzofuran; quinoline; 1,2,3,4-tetrahydronaphthalene; naphthalene; quinoxaline; 2′,3′-dihydrospiro[cyclopropane-1,1′-indene]; and pyrazole; any of which is optionally substituted with one or more substituents.

In some embodiments, for the compound or salt of Formulas (Ia), (IIa) or (IIIa), the C₃₋₁₂ carbocycle and 3- to 12-membered heterocycle of A is selected from monocyclic C₃₋₆ carbocycle and 3- to 7-membered monocyclic heterocycle, any of which is optionally substituted with one or more substituents. In some embodiments, for the compound or salt of Formulas (Ia), (IIa) or (IIIa), the C₃₋₁₂ carbocycle and 3- to 12-membered heterocycle of A is selected from phenyl, pyridine, and pyrazole, any of which is optionally substituted with one or more substituents.

In some embodiments, for the compound or salt of Formulas (Ia), (IIa) or (IIIa), the C₃₋₁₂ carbocycle and 3- to 12-membered heterocycle of A is selected from polycyclic C₇₋₁₂ carbocycle and 7- to 12-membered monocyclic heterocycle, any of which is optionally substituted with one or more substituents. In some embodiments, for the compound or salt of Formulas (Ia), (IIa) or (IIIa), the C₃₋₁₂ carbocycle and 3- to 12-membered heterocycle of A is selected from indane; chromane; benzodioxole; 2,3-dihydrobenzofuran; quinoline; 1,2,3,4-tetrahydronaphthalene; naphthalene; quinoxaline; and 2′,3′-dihydrospiro[cyclopropane-1,1′-indene]; any of which is optionally substituted with one or more substituents.

In some embodiments, for the compound or salt of Formulas (Ia), (IIa) or (IIIa), the one or more optional substituents on A are selected from:
halogen, —OR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, and —CN;
C₁₋₆ alkyl optionally substituted with one or more substituents independently selected from halogen, —OR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂ (R¹⁷), —S(O)R¹⁷, —S(O)₂R¹⁷, —S(O)₂N(R¹⁷)₂, —NO₂, ═O, —CN; and
C₃₋₁₀ carbocycle and 3- to 10-membered heterocycle, any of which is optionally substituted with one or more substituents independently selected from halogen, C₁₋₄ alkyl, C₁₋₄ haloalkyl, and ═O.

In some embodiments for the compound or salt of Formulas (Ia), (IIa) or (IIIa), the one or more optional substituents on A are selected from:
halogen, —OR¹⁷, —N(R¹⁷)₂, —C(O)R¹⁷, —C(O)OR¹⁷, —OC(O)R¹⁷, —C(O)N(R¹⁸)₂, —N(R¹⁷)C(O)R¹⁷, —N₂, ═O, and —CN;
C₁₋₆ alkyl optionally substituted with one or more substituents independently selected from halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —NO₂, ═O, —CN; and
C₃₋₁₀ carbocycle and 3- to 10-membered heterocycle, any of which is optionally substituted with one or more substituents independently selected from halogen, C₁₋₄ alkyl, C₁₋₄ haloalkyl, and ═O.

In some embodiments for the compound or salt of Formulas (Ia), (IIa) or (IIIa), the one or more optional substituents on A are selected from:
halogen, —OR¹⁷, N(R¹⁷)₂, —C(O)R¹⁷, —N(R¹⁷)C(O)R¹⁷, —N(R¹⁷)S(O)₂(R¹⁷), ═O, ═S, and —CN;
C₁₋₆ alkyl optionally substituted with one or more substituents independently selected from halogen, —OR¹⁷, —N(R¹⁷)₂, —N(R¹⁷)C(O)R¹⁷, —N(R¹⁷)S(O)₂(R¹⁷), —S(O)R¹⁷, ═O, and —CN; and
C₃₋₁₀ carbocycle and 3- to 10-membered heterocycle, any of which is optionally substituted with one or more substituents independently selected from halogen, C₁₋₄ alkyl, C₁₋₄haloalkyl, and ═O.a

In some embodiments, for the compound or salt of Formulas (Ia), (IIa) or (IIIa), the one or more optional substituents on A are selected from: halogen, —OR¹⁷, C₁₋₆alkyl, C₁₋₆ haloalkyl, C₃₋₁₀ carbocycle, and 3- to 10-membered heterocycle, wherein the C₃₋₁₀ carbocycle, and 3- to 10-membered heterocycle are each optionally substituted with one or more substituents independently selected from C₁₋₄ alkyl, C₁₋₄ haloalkyl, and ═O.

In some embodiments, for the compound or salt of Formulas (Ia), (IIa) or (IIIa), A is selected from C₃₋₁₂ carbocycle and 3- to 12-membered heterocycle, any of which is optionally substituted with one or more substituents selected from:
halogen, —OR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, and —CN;
C₁₋₆ alkyl optionally substituted with one or more substituents independently selected from halogen, —OR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, —CN; and
C₃₋₁₀ carbocycle and 3- to 10-membered heterocycle, any of which is optionally substituted with one or more substituents independently selected from halogen, C₁₋₄alkyl, C₁₋₄ haloalkyl, and ═O.

In some embodiments, for the compound or salt of Formulas (Ia), (IIa) or (IIa), A is selected from C₃₋₁₂ carbocycle and 3- to 12-membered heterocycle, any of which is optionally substituted with one or more substituents selected from: halogen; —OR¹⁷; C₁₋₆alkyl optionally substituted with one or more substituents independently selected from halogen; and C₃₋₁₀ carbocycle and 3- to 10-membered heterocycle, any of which is optionally substituted with one or more substituents independently selected from halogen, C₁₋₄ alkyl, C₁₋₄ haloalkyl, and ═O. In some embodiments, A is selected from C₃₋₁₂ carbocycle and 3- to 12-membered heterocycle, any of which is optionally substituted with one or more substituents selected from: halogen, —OR¹⁷, C₁₋₆ alkyl, C₁₋₆haloalkyl, C₃₋₁₀ carbocycle, and 3- to 10-membered heterocycle, wherein the C₃₋₁₀ carbocycle, and 3- to 10-membered heterocycle are each optionally substituted with one or more substituents independently selected from C₁₋₄ alkyl, C₁₋₄ haloalkyl, and ═O. In some embodiments, R¹⁷ is independently selected at each occurrence from hydrogen; C₁₋₆ alkyl optionally substituted with one or more substituents independently selected from: halogen, —OR²¹, —N(R²¹)₂, —C(O)N(R²¹)₂, —N(R²¹)C(O)R²¹, ═O, and —CN; and C₃₋₆ carbocycle and 3- to 6-membered heterocycle, any of which is optionally substituted with one or more substituents independently selected from: halogen, C₁₋₄ alkyl, C₁₋₄ haloalkyl, —OR²¹, —N(R²¹)₂, —C(O)R²¹, —C(O)N(R²¹)₂, —N(R²¹)C(O)R²¹, ═O, and —CN. In some embodiments, R¹⁷ is independently selected at each occurrence from hydrogen, C₁₋₆ alkyl, C₃₋₆ carbocycle and 3- to 6-membered heterocycle.

In some embodiments, for the compound or salt of Formulas (Ia), (IIa) or (IIIa), A is selected from C₃₋₂ carbocycle and 3- to 12-membered heterocycle, any of which is optionally substituted with one or more substituents selected from: halogen, hydroxyl, methoxy, trifluoromethyl, propyl, cyclopropyl, cyclopentyl, phenyl, phenoxy,

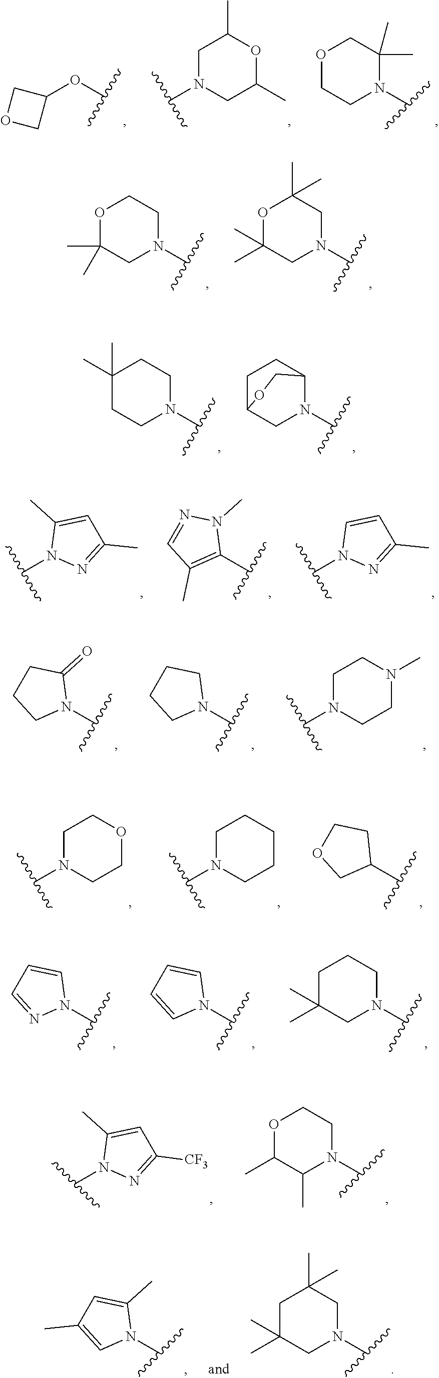

In some embodiments, for the compound or salt of Formulas (Ia), (IIa) or (IIIa), A is selected from C₃₋₁₂ carbocycle and 3- to 12-membered heterocycle, any of which is optionally substituted with one or more substituents selected from: halogen, hydroxyl, methoxy, trifluoromethyl, propyl, cyclopropyl, cyclopentyl, phenyl, phenoxy,

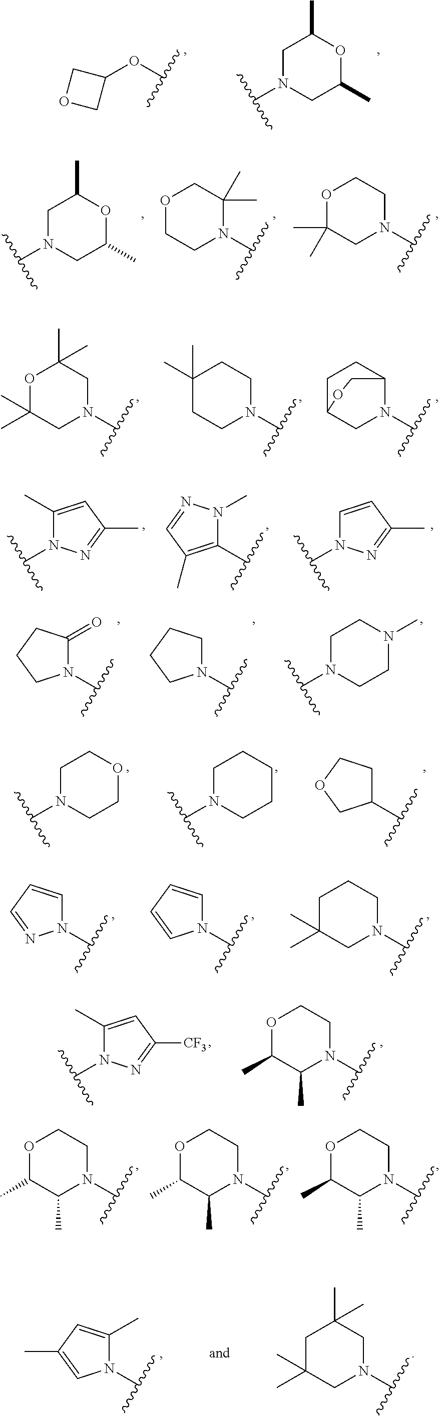

In some embodiments, for the compound or salt of Formulas (Ia), (IIa) or (IIIa), A is selected from phenyl; pyridine; indane; chromane; benzodioxole; 2,3-dihydrobenzofuran; quinoline; 1,2,3,4-tetrahydronaphthalene; naphthalene; quinoxaline; 2′,3′-dihydrospiro[cyclopropane-1,1′-indene]; and pyrazole; any of which is optionally substituted with one or more substituents independently selected from:
halogen, —OR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, and —CN;
C₁₋₆ alkyl optionally substituted with one or more substituents independently selected from halogen, —OR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, —CN; and
C₃₋₁₀ carbocycle and 3- to 10-membered heterocycle, any of which is optionally substituted with one or more substituents independently selected from halogen, C₁₋₄alkyl, C₁₋₄ haloalkyl, and ═O.

In some embodiments, for the compound or salt of Formulas (Ia), (IIa) or (IIIa), A is selected from phenyl; pyridine; indane; chromane; benzodioxole; 2,3-dihydrobenzofuran; quinoline; 1,2,3,4-tetrahydronaphthalene; naphthalene; quinoxaline; 2′,3′-dihydrospiro[cyclopropane-1,1′-indene]; and pyrazole; any of which is optionally substituted with one or more substituents independently selected from:
halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —NO₂, ═O, and —CN;
C₁₋₆ alkyl optionally substituted with one or more substituents independently selected from halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —NO₂, ═O, —CN; and
C₃₋₁₀ carbocycle and 3- to 10-membered heterocycle, any of which is optionally substituted with one or more substituents independently selected from halogen, C₁₋₄ alkyl, C₁₋₄haloalkyl, and ═O.

In some embodiments, for the compound or salt of Formulas (Ia), (IIa) or (IIa), A is selected from phenyl; pyridine; indane; chromane; benzodioxole; 2,3-dihydrobenzofuran; quinoline; 1,2,3,4-tetrahydronaphthalene; naphthalene; quinoxaline; 2′,3′-dihydrospiro[cyclopropane-1,1′-indene]; and pyrazole; any of which is optionally substituted with one or more substituents independently selected from: halogen; —OR¹⁷; C₁₋₆ alkyl optionally substituted with one or more substituents independently selected from halogen; and C₃₋₁₀ carbocycle and 3- to 10-membered heterocycle, any of which is optionally substituted with one or more substituents independently selected from halogen, C₁₋₄ alkyl, C₁₋₄ haloalkyl, and ═O.

In some embodiments, for the compound or salt of Formulas (Ia), (IIa) or (IIIa), A is selected from phenyl; pyridine; indane; chromane; benzodioxole; 2,3-dihydrobenzofuran; quinoline; 1,2,3,4-tetrahydronaphthalene; naphthalene; quinoxaline; 2′,3′-dihydrospiro[cyclopropane-1,1′-indene]; and pyrazole; any of which is optionally substituted with one or more substituents independently selected from: halogen, —OR¹⁷, C₁₋₆alkyl, C₁₋₆haloalkyl, C₃₋₁₀ carbocycle, and 3- to 10-membered heterocycle, wherein the C₃₋₁₀ carbocycle, and 3- to 10-membered heterocycle are each optionally substituted with one or more substituents independently selected from C₁₋₄alkyl, C₁₋₄ haloalkyl, and ═O.

In some embodiments, for the compound or salt of Formulas (Ia), (IIa) or (ilia), A is selected from phenyl; pyridine, indane; chromane, benzodioxole; 2,3-dihydrobenzofuran; quinoline; 1,2,3,4-tetrahydronaphthalene; naphthalene; quinoxaline; 2′,3′-dihydrospiro[cyclopropane-1,1′-indene]; and pyrazole; any of which is optionally substituted with one or more substituents independently selected from: halogen, hydroxyl, methoxy, trifluoromethyl, propyl, cyclopropyl, cyclopentyl, phenyl, phenoxy,

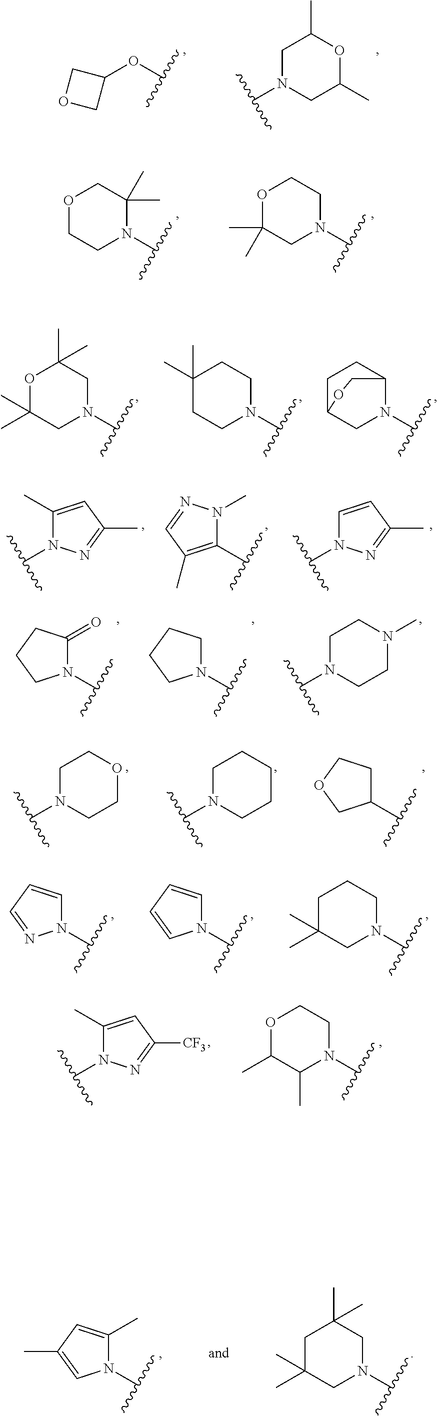

In some embodiments, for the compound or salt of Formulas (Ia), (IIa) or (IIIa), A are selected from phenyl; pyridine, indane; chromane, benzodioxole; 2,3-dihydrobenzofuran, quinoline; 1,2,3,4-tetrahydronaphthalene, naphthalene; quinoxaline; 2′,3′-dihydrospiro[cyclopropane-1,1′-indene]; and pyrazole; any of which is optionally substituted with one or more substituents independently selected from: halogen, hydroxyl, methoxy, trifluoromethyl, propyl, cyclopropyl, cyclopentyl, phenyl, phenoxy,

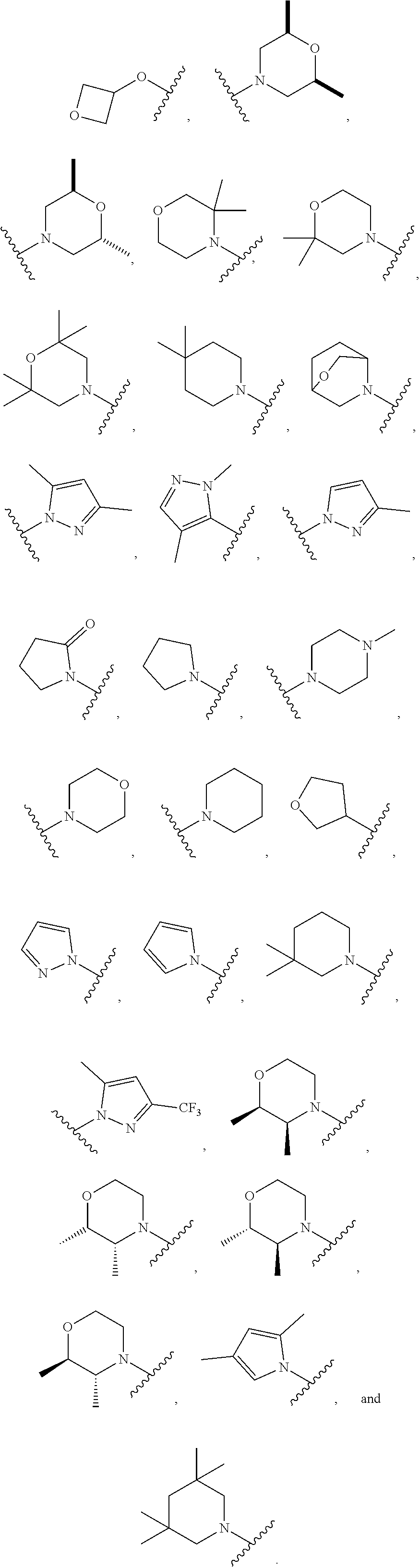

In some embodiments, for the compound or salt of Formulas (Ia), (IIa) or (IIIa), the C₃₋₁₂ carbocycle and 3- to 12-membered heterocycle of A is selected from monocyclic C₃₋₆ carbocycle and 3- to 7-membered monocyclic heterocycle, any of which is optionally substituted with one or more substituents independently selected from:
halogen, —OR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, and —CN;
C₁₋₆ alkyl optionally substituted with one or more substituents independently selected from halogen, —OR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷. —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R, —S(O)₂N(R¹⁷)₂, —NO₂, ═O, —CN; and
C₃₋₆ carbocycle and 3- to 10-membered heterocycle, any of which is optionally substituted with one or more substituents independently selected from halogen, C₁₋₄ alkyl, C₁₋₄ haloalkyl, and ═O.

In some embodiments, for the compound or salt of Formulas (Ia), (IIa) or (IIa), the C₃₋₁₀ carbocycle and 3- to 12-membered heterocycle of A is selected from monocyclic C₃₋₆ carbocycle and 3- to 7-membered monocyclic heterocycle, any of which is optionally substituted with one or more substituents independently selected from:
halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —NO₂, ═O, and —CN;
C₁₋₆ alkyl optionally substituted with one or more substituents independently selected from halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —NO₂, ═O, —CN; and
C₃₋₁₀ carbocycle and 3- to 10-membered heterocycle, any of which is optionally substituted with one or more substituents independently selected from halogen, C₁₋₄ alkyl, C₁₋₄haloalkyl, and ═O.

In some embodiments, for the compound or salt of Formulas (Ia), (IIa) or (IIIa), the C₃₋₁₂ carbocycle and 3- to 12-membered heterocycle of A is selected from monocyclic C₃₋₆ carbocycle and 3- to 7-membered monocyclic heterocycle, any of which is optionally substituted with one or more substituents independently selected from: halogen, —OR¹⁷, C₁₋₆ alkyl, C₁₋₆ haloalkyl, C₃₋₁₀ carbocycle, and 3- to 10-membered heterocycle, wherein the C₃₋₁₀ carbocycle, and 3- to 10-membered heterocycle are each optionally substituted with one or more substituents independently selected from C₁₋₄ alkyl, C₁₋₄ haloalkyl, and ═O.

In some embodiments, for the compound or salt of Formulas (Ia), (IIa) or (IIIa), the C₃₋₁₂ carbocycle and 3- to 12-membered heterocycle of A is selected from monocyclic C₃₋₆ carbocycle and 3- to 7-membered monocyclic heterocycle, any of which is optionally substituted with one or more substituents independently selected from: halogen, hydroxyl, methoxy, trifluoromethyl, propyl, cyclopropyl, cyclopentyl, phenyl, phenoxy,

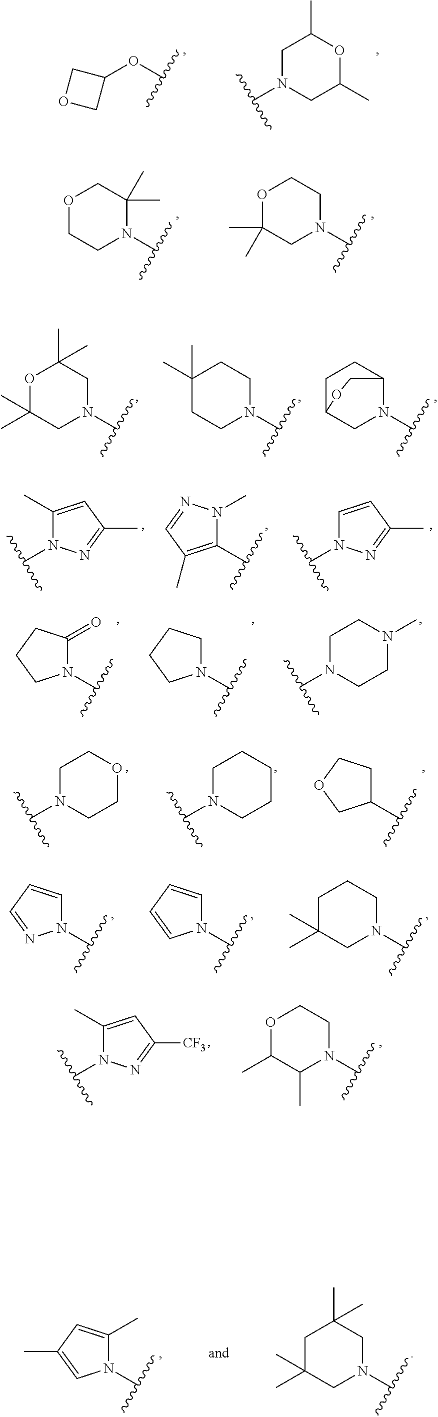

In some embodiments, for the compound or salt of Formulas (Ia), (IIa) or (IIIa), the C₃₋₁₂ carbocycle and 3- to 12-membered heterocycle of A is selected from phenyl, pyridine, and pyrazole, any of which is optionally substituted with one or more substituents independently selected from:
halogen, —OR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, and —CN;
C₁₋₆ alkyl optionally substituted with one or more substituents independently selected from halogen, —OR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R⁴⁷)₂, —N(R¹⁷)S(O)₂(R¹⁷), —S(O)₂R¹⁷, —S(O)₂R¹⁷, —S(O)₂N(R¹⁷)₂, —NO₂, ═O, —CN; and
C₃₋₁₀ carbocycle and 3- to 10-membered heterocycle, any of which is optionally substituted with one or more substituents independently selected from halogen, C₁₋₄ alkyl, C₁₋₄ haloalkyl, and ═O.

In some embodiments, for the compound or salt of Formulas (Ia), (IIa) or (IIIa), the C₃₋₁₀ carbocycle and 3- to 12-membered heterocycle of A is selected from phenyl, pyridine, and pyrazole, any of which is optionally substituted with one or more substituents independently selected from:
halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —NO₂, ═O, and —CN;
C₁₋₆ alkyl optionally substituted with one or more substituents independently selected from halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —NO₂, ═O, —CN; and
C₃₋₁₀ carbocycle and 3- to 10-membered heterocycle, any of which is optionally substituted with one or more substituents independently selected from halogen, C₁₋₄ alkyl, C₁₋₄haloalkyl, and ═O.

In some embodiments, for the compound or salt of Formulas (Ia), (IIa) or (IIIa), the C₃₋₁₂ carbocycle and 3- to 12-membered heterocycle of A is selected from phenyl, pyridine, and pyrazole, any of which is optionally substituted with one or more substituents independently selected from: halogen, —OR¹⁷, C₁₋₆ alkyl, C₁₋₆haloalkyl, C₃₋₁₀ carbocycle, and 3- to 10-membered heterocycle, wherein the C₃₋₁₀ carbocycle, and 3- to 10-membered heterocycle are each optionally substituted with one or more substituents independently selected from C₁₋₄ alkyl, C₁₋₄ haloalkyl, and ═O.

In some embodiments, for the compound or salt of Formulas (Ia), (IIa) or (IIIa), the C₃₋₁₀ carbocycle and 3- to 12-membered heterocycle of A is selected from phenyl, pyridine, and pyrazole, any of which is optionally substituted with one or more substituents independently selected from: halogen, hydroxyl, methoxy, trifluoromethyl, propyl, cyclopropyl, cyclopentyl, phenyl, phenoxy,

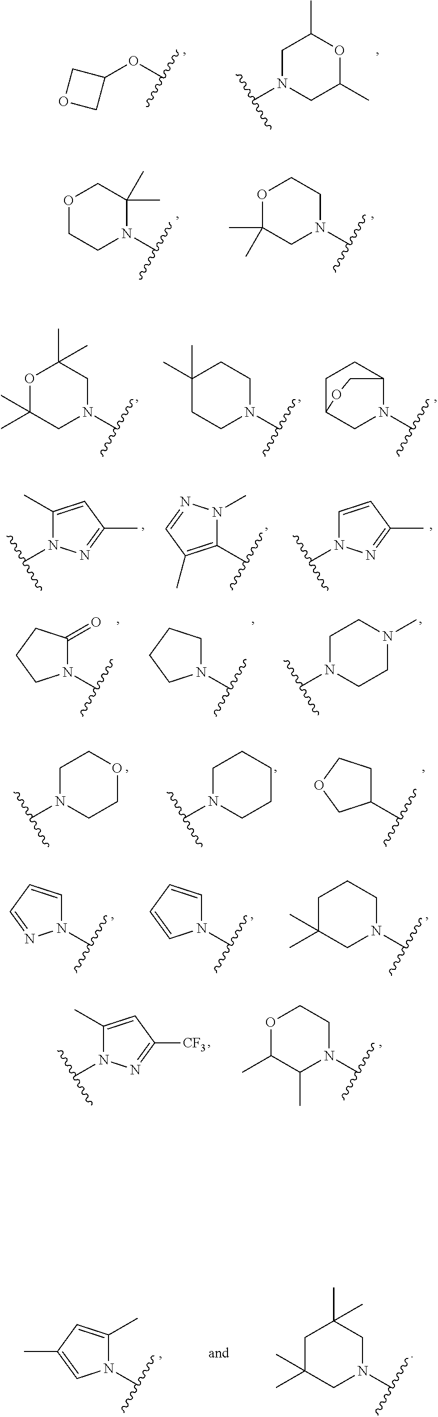

In some embodiments, for the compound or salt of Formulas (Ia), (IIa) or (IIIa), the C₃₋₁₂ carbocycle and 3- to 12-membered heterocycle of A is selected from polycyclic C₇₋₁₂ carbocycle and 7- to 12-membered polycyclic heterocycle, any of which is optionally substituted with one or more substituents independently selected from:
halogen, —OR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹²)₂, —N(R¹⁷)S(O)₂(R¹⁷), —S(O)R¹⁷, —S(O)₂R¹⁷, —S(O)₂N(R¹⁷)₂, —NO₂, ═O, and —CN;
C₁₋₆ alkyl optionally substituted with one or more substituents independently selected from halogen, —OR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, —CN; and
C₃₋₁₀ carbocycle and 3- to 10-membered heterocycle, any of which is optionally substituted with one or more substituents independently selected from halogen, C₁₋₄ alkyl, C₁₋₄ haloalkyl, and ═O.

In some embodiments, for the compound or salt of Formulas (Ia), (IIa) or (IIIa), the C₃₋₁₀ carbocycle and 3- to 12-membered heterocycle of A is selected from polycyclic C₇₋₁₂ carbocycle and 7- to 12-membered polycyclic heterocycle, any of which is optionally substituted with one or more substituents independently selected from:
halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —NO₂, ═O, and —CN;
C₁₋₆ alkyl optionally substituted with one or more substituents independently selected from halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —NO₂, ═O, —CN; and
C₃₋₁₀ carbocycle and 3- to 10-membered heterocycle, any of which is optionally substituted with one or more substituents independently selected from halogen, C₁₋₄ alkyl, C₁₋₄ haloalkyl, and ═O.

In some embodiments, for the compound or salt of Formulas (Ia), (IIa) or (IIIa), the C₃₋₂ carbocycle and 3- to 12-membered heterocycle of A is selected from polycyclic C₇-12 carbocycle and 7- to 12-membered polycyclic heterocycle, any of which is optionally substituted with one or more substituents independently selected from: halogen, —OR¹⁷, C₁₋₆alkyl, C₁₋₆ haloalkyl, C₃₋₁₀ carbocycle, and 3- to 10-membered heterocycle, wherein the C₃₋₁₀ carbocycle, and 3- to 10-membered heterocycle are each optionally substituted with one or more substituents independently selected from C₁₋₄ alkyl, C₁₋₄ haloalkyl, and ═O.

In some embodiments, for the compound or salt of Formulas (Ia), (IIa) or (IIIa), the C₃₋₁₂ carbocycle and 3- to 12-membered heterocycle of A is selected from polycyclic C₇₋₁₂ carbocycle and 7- to 12-membered polycyclic heterocycle, any of which is optionally substituted with one or more substituents independently selected from: halogen, hydroxyl, methoxy, trifluoromethyl, propyl, cyclopropyl, cyclopentyl, phenoxy,

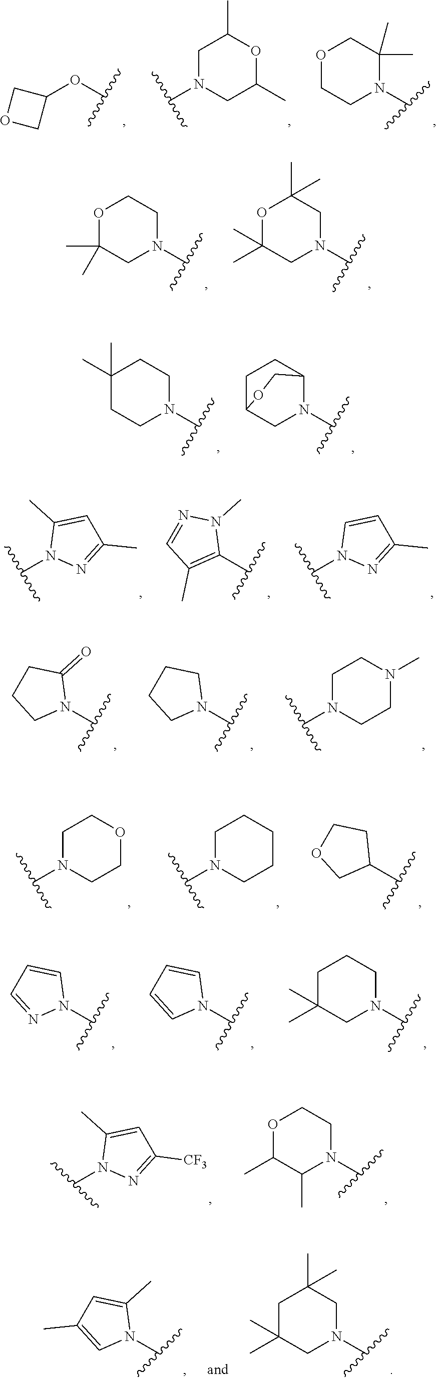

In some embodiments, for the compound or salt of Formulas (Ia), (IIa) or (IIIa), the C₃₋₁₂ carbocycle and 3- to 12-membered heterocycle of A is selected from indane; chromane; benzodioxole; 2,3-dihydrobenzofuran; quinoline; 1,2,3,4-tetrahydronaphthalene; naphthalene; quinoxaline; and 2′,3′-dihydrospiro[cyclopropane-1,1′-indene]; any of which is optionally substituted with one or more substituents independently selected from:
halogen, —OR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R₁₇, —S(O)₂N(R¹⁷)₂, —NO₂, ═O, and —CN;
C₁₋₆ alkyl optionally substituted with one or more substituents independently selected from halogen, —OR¹⁷, —N(R¹⁷)₂, —C(O)R¹⁷, —C(O)OR¹⁷, —OC(O)R¹⁷—, OC(O)N(R¹⁷)₂, —C(O)N(R¹⁷)₂, —N(R¹⁷)C(O)R¹⁷, —N(R¹⁷)C(O)OR 1, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, —CN; and
C₃₋₁₀ carbocycle and 3- to 10-membered heterocycle, any of which is optionally substituted with one or more substituents independently selected from halogen, C₁₋₄ alkyl, C₁₋₄ haloalkyl, and ═O.

In some embodiments, for the compound or salt of Formulas (Ia), (IIa) or (IIa), the C₃₋₁₀ carbocycle and 3- to 12-membered heterocycle of A is selected from indane; chromane; benzodioxole; 2,3-dihydrobenzofuran; quinoline; 1,2,3,4-tetrahydronaphthalene; naphthalene; quinoxaline; and 2′,3′-dihydrospiro[cyclopropane-1,1′-indene]; any of which is optionally substituted with one or more substituents independently selected from:
halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —NO₂, ═O, and —CN;
C₁₋₆ alkyl optionally substituted with one or more substituents independently selected from halogen, —OR¹⁷, —N(R¹⁷)₂, —C(O)R¹⁷, —C(O)OR¹⁷, —OC(O)R¹⁷, —C(O)N(R¹⁷)₂, —N(R¹⁷)C(O)R¹⁷, —NO₂, ═O, —CN; and
   C₃₋₁₀ carbocycle and 3- to 10-membered heterocycle, any of which is optionally substituted with one or more substituents independently selected from halogen, C₁₋₄ alkyl, C₁₋₄ haloalkyl, and ═O.

In some embodiments, for the compound or salt of Formulas (Ia), (IIa) or (IIIa), the C₃₋₂ carbocycle and 3- to 12-membered heterocycle of A is selected from indane; chromane; benzodioxole; 2,3-dihydrobenzofuran; quinoline; 1,2,3,4-tetrahydronaphthalene; naphthalene; quinoxaline; and 2′,3′-dihydrospiro[cyclopropane-1,1′-indene]; any of which is optionally substituted with one or more substituents independently selected from: halogen, —OR¹⁷, C₁₋₆ alkyl, C₁₋₆haloalkyl, C₃₋₁₀ carbocycle, and 3- to 10-membered heterocycle, wherein the C₃₋₁₀ carbocycle, and 3- to 10-membered heterocycle are each optionally substituted with one or more substituents independently selected from C₁₋₄ alkyl, C₁₋₄ haloalkyl, and ═O.

In some embodiments, for the compound or salt of Formulas (Ia), (IIa) or (IIIa), the C₃₋₁₀ carbocycle and 3- to 12-membered heterocycle of A is selected from indane; chromane; benzodioxole; 2,3-dihydrobenzofuran; quinoline; 1,2,3,4-tetrahydronaphthalene; naphthalene; quinoxaline; and 2′,3′-dihydrospirolcyclopropane-1,1′-indane; any of which is optionally substituted with one or more substituents independently selected from: halogen, hydroxyl, methoxy, trifluoromethyl, propyl, cyclopropyl, cyclopentyl, phenyl, phenoxy,

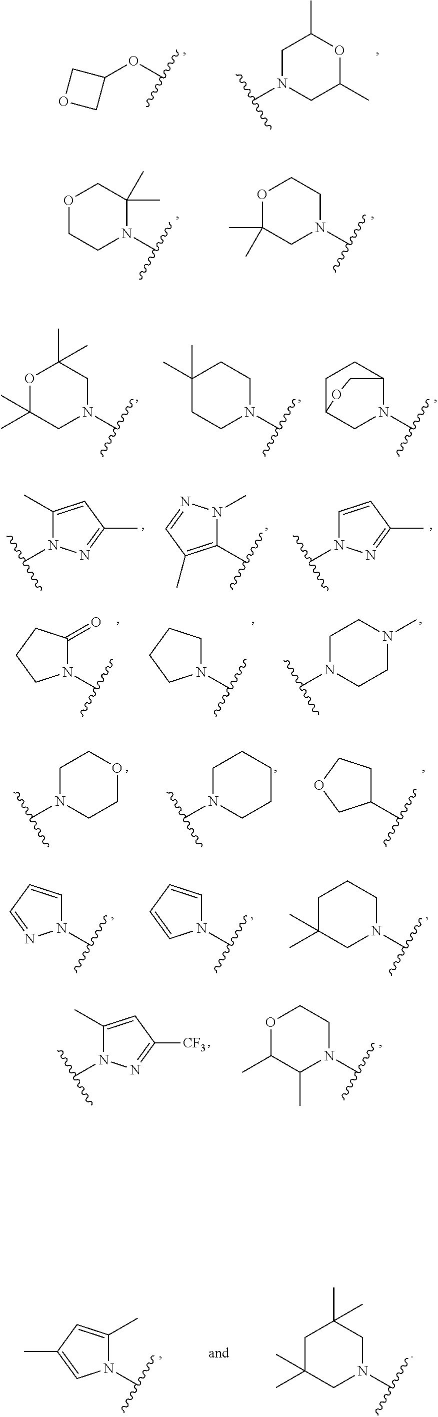

In some embodiments, for the compound or salt of Formulas (Ia), (IIa) or (IIIa), A is selected from:

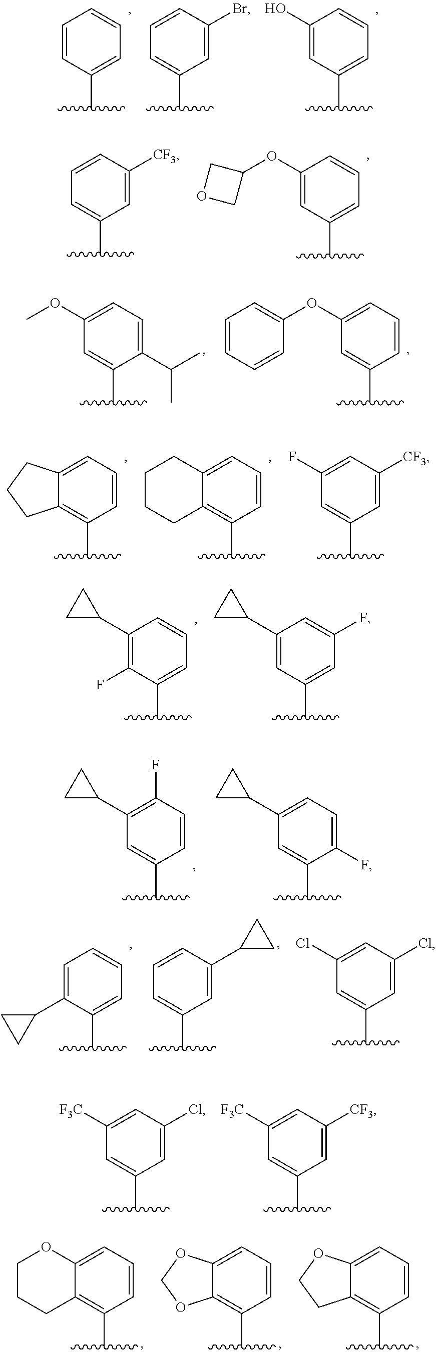

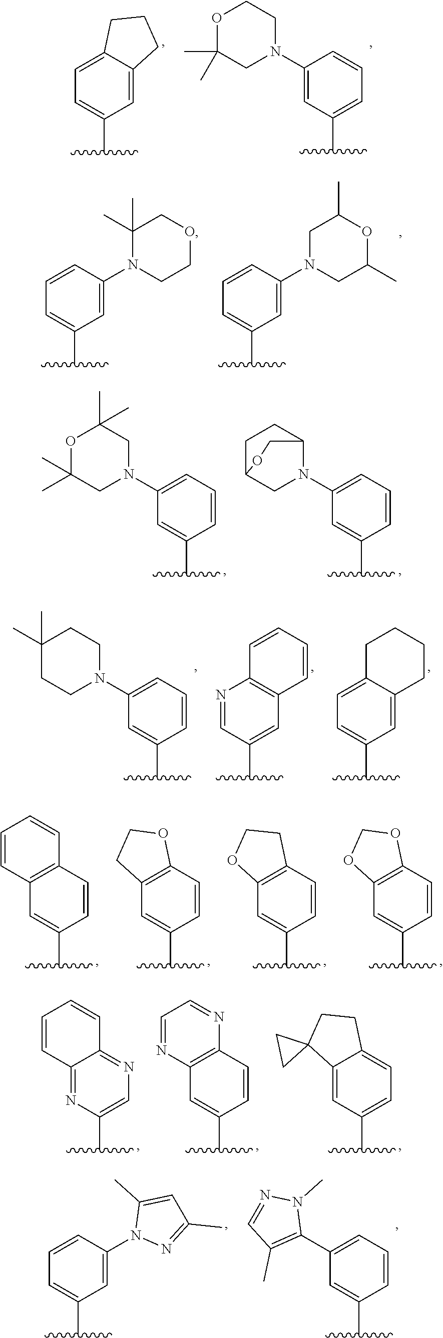

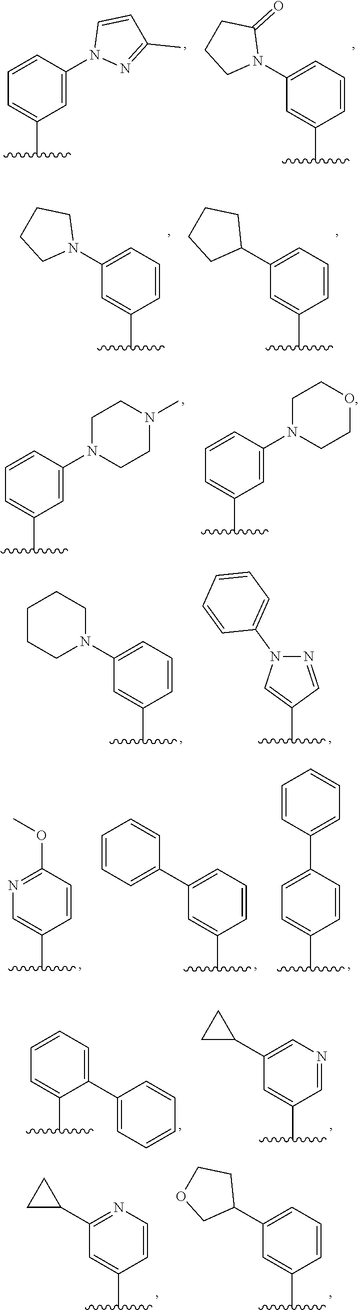

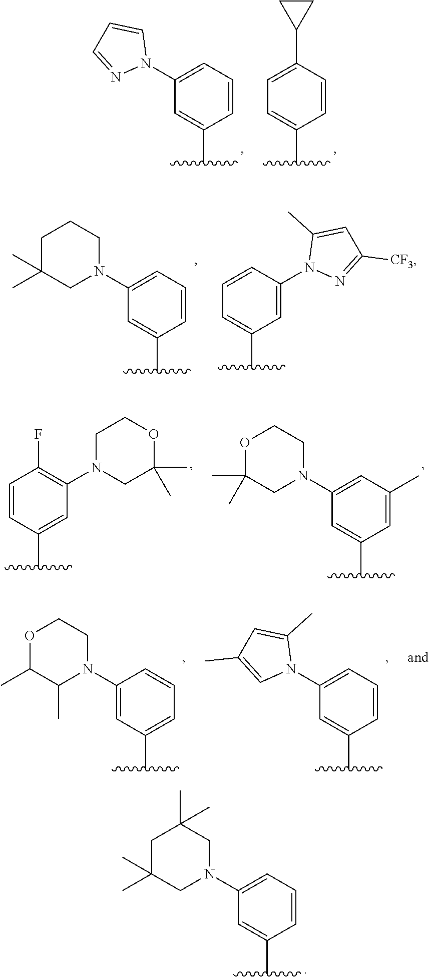

In some embodiments, for the compound or salt of Formulas (Ia), (IIa) or (IIIa), A is selected from:

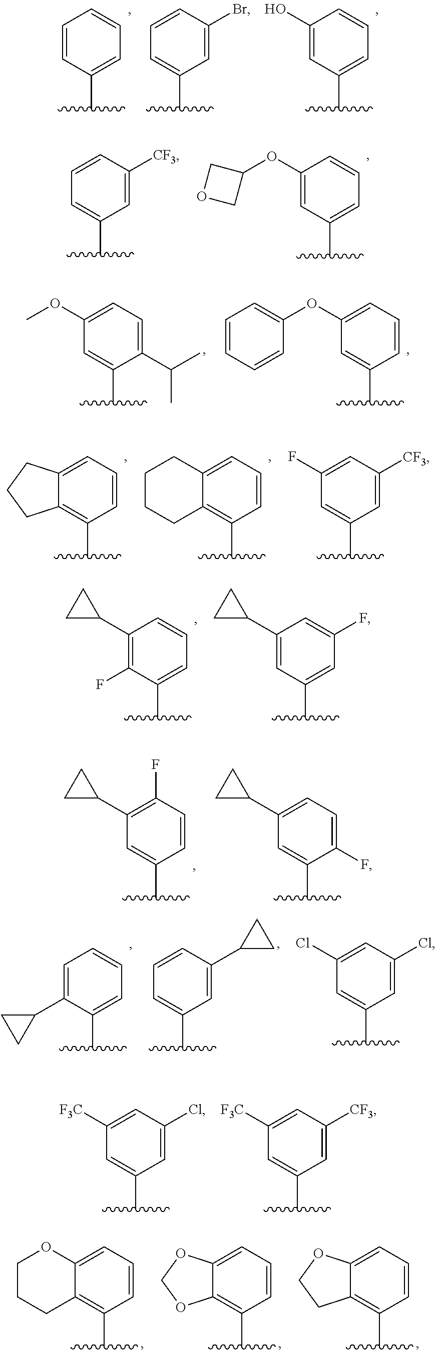

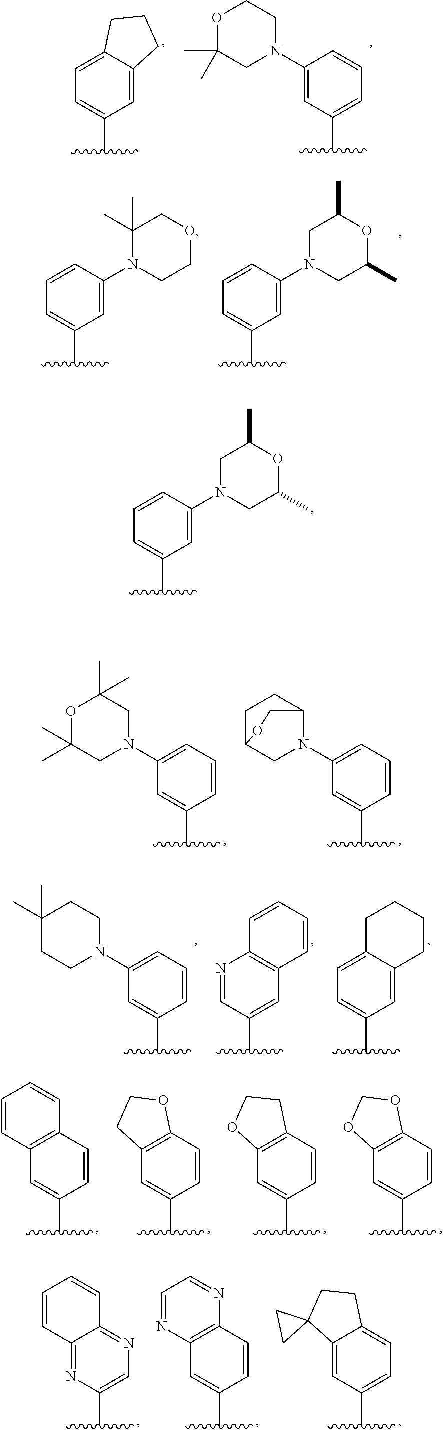

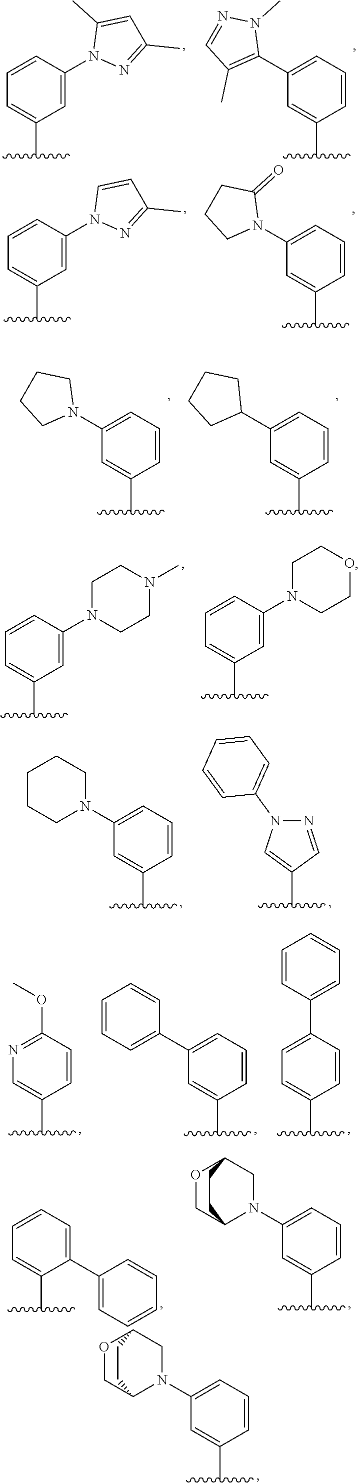

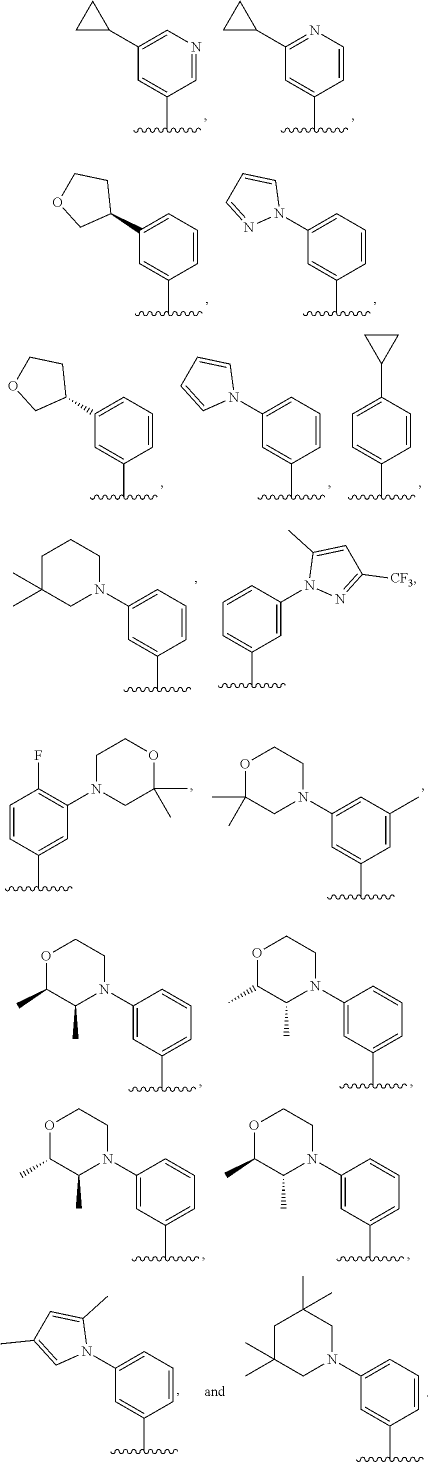

In some embodiments, for the compound or salt of Formulas (Ia), (IIa) or (IIa), B is selected from (I) when A is selected from (ii), or B is selected from (II) when A is selected from (i):
(I) hydrogen, halogen, and —CN, or B and R⁷ are taken together to form a C₃₋₆ carbocycle or a 3- to 6-membered heterocycle;
(II) —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, and —S(O)₂N(R¹⁸)₂;
   C₃₋₁₂ carbocycle and 3- to 12-membered heterocycle, any of which is optionally substituted with one or more substituents independently selected from:
      halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R'S)₂, —N(R¹⁸)C(S)N(R¹⁸)₂, —N(R¹⁸)S(O)₂(R¹⁸), —S(O)R¹³, —S(O)₂R¹⁸, —S(O)₂N(R¹⁸)₂, —NO₂, ═O, ═S, ═N(R¹⁸), —N₃, and —CN,
      C₁₋₆ alkyl optionally substituted with one or more substituents independently selected from:
         halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═N(R¹⁸), —N₃, —CN, C₃₋₆ carbocycle and 3- to 6-membered heterocycle,
         wherein the C₃₋₆ carbocycle and 3- to 6-membered heterocycle are each optionally substituted with one or more substituents independently selected from halogen. C₁₋₄ alkyl, C₁₋₄ haloalkyl, and ═O; and
         C₃₋₁₀ carbocycle and 3- to 10-membered heterocycle, any of which is optionally substituted with one or more substituents independently selected from halogen, C₁₋₄alkyl, C₁₋₄ haloalkyl, and ═O.

In some embodiments, for the compound or salt of Formulas (Ia), (IIa) or (IIIa), B is selected from (1) when A is selected from (ii), or B is selected from (II) when A is selected from (i):
(I) hydrogen, halogen, and —CN, or B and R⁷ are taken together to form a C₃₋₆ carbocycle or a 3- to 6-membered heterocycle,
(II) —OR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)S(O)₂(R¹⁸), —S(O)₂R¹⁸, and —S(O)₂N(R¹⁸)₂:
   C₃₋₁₂ carbocycle and 3- to 12-membered heterocycle, any of which is optionally substituted with one or more substituents independently selected from:
      halogen, —OR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)S(O)₂(R¹⁸), —S(O)R¹⁸, —S(O)₂R¹⁸, —S(O)₂N(R¹⁸)₂, ═O, and —CN;
      C₁₋₆ alkyl optionally substituted with one or more substituents independently selected from:
         halogen, —OR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)S(O)₂(R¹⁸), —S(O)R¹⁸, —S(O)₂R¹⁸, —S(O)₂N(R¹⁸)₂, ═O—CN, C₃₋₆ carbocycle and 3- to 6-membered heterocycle,
         wherein the C₃ carbocycle and 3- to 6-membered heterocycle are each optionally substituted with one or more substituents independently selected from halogen, C₁₋₄alkyl, C₁₋₄ haloalkyl, and ═O; and
         C₃₋₁₀ carbocycle and 3- to 10-membered heterocycle, any of which is optionally substituted with one or more substituents independently selected from halogen, C₁₋₄alkyl, C₁₋₄ haloalkyl, and ═O.

In some embodiments, for the compound or salt of Formulas (Ia), (IIa) or (IIIa), B is selected from (I) when A is selected from (ii), or B is selected from (II) when A is selected from (i):
(I) hydrogen and halogen;
(II) —N(R¹⁸)₂, —C(O)R¹⁸, —C(O)OR¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)S(O)₂(R¹⁸):
   C₃₋₁₂ carbocycle and 3- to 12-membered heterocycle, any of which is optionally substituted with one or more substituents independently selected from:
      halogen, —OR¹⁸, —N(R¹⁸)₂, —C(O)R¹⁸, —C(O)OR¹⁸, —C(O)N(R¹⁸)₂, —N(R¹⁸)C(O)R¹⁸, —N(R¹⁸)S(O)₂(R¹⁸), —S(O)₂R¹⁸, ═O, and —CN;
      C₁₋₆ alkyl optionally substituted with one or more substituents independently selected from:
         halogen, —OR¹⁸, —N(R¹⁸)₂, —C(O)R¹⁸, —C(O)OR¹⁸, —C(O)N(R¹⁸)₂, —N(R¹⁸)C(O)R¹⁸, —N(R¹⁸)S(O)₂(R¹⁸), —S(O)₂R¹⁸, ═O, —CN, and C₃₋₆ carbocycle, optionally substituted with one or more substituents independently selected from halogen, C₁₋₄ alkyl, C₁₋₄ haloalkyl, and ═O; and
      C₃₋₁₀ carbocycle and 3- to 10-membered heterocycle, any of which is optionally substituted with one or more substituents independently selected from halogen, C₁₋₄ alkyl, C₁₋₄ haloalkyl, and ═O.

In some embodiments, for the compound or salt of Formulas (Ia), (IIa) or (IIIa), B is selected from (I) when A is selected from (ii), or B is selected from (II) when A is selected from (i):
(I) hydrogen and halogen;
(II) —N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)S(O)₂(R¹⁸); and
   C₃₋₁₂ carbocycle and 3- to 12-membered heterocycle, any of which is optionally substituted with one or more substituents independently selected from:
      halogen; —OR¹⁸; C₁₋₆alkyl optionally substituted with one or more substituents independently selected from halogen, —OR¹⁸, and C₃₋₆ carbocycle; and C₃₋₁₀ carbocycle and 3- to 10-membered heterocycle, any of which is optionally substituted with one or more substituents independently selected from halogen and C₁₋₄ alkyl.

In some embodiments, for the compound or salt of Formulas (Ia), (IIa) or (IIIa), B is selected from (I) when A is selected from (ii), or B is selected from (II) when A is selected from (i):
(I) hydrogen and halogen;
(II) —N(R¹⁸)₂, —N(R¹⁸)C(O)R¹⁸, —N(R¹⁸)C(O)OR¹⁸, —N(R¹⁸)C(O)N(R¹⁸)₂, and —N(R¹⁸)S(O)₂(R¹⁸).

In some embodiments, for the compound or salt of Formulas (Ia), (IIa) or (IIIa), B is selected from (I) when A is selected from (ii), or B is selected from (II) when A is selected from (i):
(I) hydrogen and halogen;
(II) C₃₋₁₂ carbocycle and 3- to 12-membered heterocycle, any of which is optionally substituted with one or more substituents independently selected from:
   halogen; —OR¹⁸; C₁₋₆ alkyl optionally substituted with one or more substituents independently selected from halogen, —OR¹⁸, and C₃₋₆ carbocycle; and C₃₋₁₀ carbocycle and 3- to 10-membered heterocycle, any of which is optionally substituted with one or more substituents independently selected from halogen and C₁₋₄ alkyl.

In some embodiments, for the compound or salt of Formulas (Ia), (IIa) or (IIIa), B is selected from hydrogen, halogen, and —CN, or B and R⁷ are taken together to form a C₃₋₆ carbocycle or a 3- to 6-membered heterocycle. In some embodiments, B is selected from hydrogen and halogen, or B and R⁷ are taken together to form a C₃₋₆ carbocycle or a 3- to 6-membered heterocycle. In some embodiments, B is selected from hydrogen and halogen, or B and R⁷ are taken together to form a C₃₋₆ carbocycle. In some embodiments, B is selected from hydrogen, halogen, and —CN. In some embodiments, B is selected from hydrogen and halogen. In some embodiments, B is selected from hydrogen and halogen. In some embodiments, B is selected from hydrogen. In some embodiments, B is selected from halogen. In some embodiments, B and R⁷ are taken together to form a C₃₋₆ carbocycle.

In some embodiments, for the compound or salt of Formulas (Ia), (IIa) or (IIIa), B is selected from C₃₋₁₂ carbocycle and 3- to 12-membered heterocycle, any of which is optionally substituted with one or more substituents.

In some embodiments, for the compound or salt of Formulas (Ia), (IIa) or (IIIa), the C₃₋₁₂ carbocycle and 3- to 12-membered heterocycle of B are each optionally substituted with one or more substituents independently selected from:
halogen, —OR¹⁸, —N(R¹⁸)₂, —C(O)R¹⁸, and ═O;
C₁₋₆ alkyl optionally substituted with one or more substituents independently selected from halogen, —OR¹⁸, —N(R¹⁸)₂, ═O, —CN, C₃₋₆ carbocycle, and 3- to 6-membered heterocycle, wherein the C₃₋₆ carbocycle and 3- to 6-membered heterocycle are each optionally substituted with one or more substituents independently selected from halogen, C₁₋₄alkyl, C₁₋₄haloalkyl, and ═O; and C₃₋₁₀ carbocycle and 3- to 10-membered heterocycle, any of which is optionally substituted with one or more substituents independently selected from halogen, C₁₋₄ alkyl, C₁₋₄haloalkyl, and ═O.

In some embodiments, for the compound or salt of Formulas (Ia), (IIa) or (IIIa), the C₃₋₁₂ carbocycle and 3- to 12-membered heterocycle of B are each optionally substituted with one or more substituents independently selected from: halogen; —OR¹⁸; C₁₋₆ alkyl optionally substituted with one or more substituents independently selected from halogen, OR¹⁸, and C₃₋₆ carbocycle, and C₃₋₁₀ carbocycle and 3- to 10-membered heterocycle, wherein the C₃₋₁₀ carbocycle and 3- to 10-membered heterocycle are each optionally substituted with one or more substituents independently selected from halogen and C₁₋₄ alkyl.

In some embodiments, for the compound or salt of Formulas (Ia), (IIa) or (IIIa), B is selected from C₃₋₁₂ carbocycle and 3- to 12-membered heterocycle, any of which is optionally substituted with one or more substituents independently selected from:
halogen, —OR¹⁸, —N(R¹⁸)₂, —C(O)R¹⁸, ═O, and —CN;
C₁₋₆ alkyl optionally substituted with one or more substituents independently selected from halogen, —OR¹⁸, —SR¹⁸, —N(R¹⁸)₂, —C(O)R¹⁸, ═O, —CN, C₃₋₆ carbocycle, and 3- to 6-membered heterocycle, wherein the C₃₋₆ carbocycle and 3- to 6-membered heterocycle are each optionally substituted with one or more substituents independently selected from halogen, C₁₋₄ alkyl, C₁₋₄ haloalkyl, and ═O; and C₃₋₁₀ carbocycle and 3- to 10-membered heterocycle, any of which is optionally substituted with one or more substituents independently selected from halogen, C₁₋₄ alkyl, C₁₋₄haloalkyl, and ═O

In some embodiments, for the compound or salt of Formulas (Ia), (IIa) or (IIIa), B is selected from C₃₋₁₂ carbocycle and 3- to 12-membered heterocycle, any of which is optionally substituted with one or more substituents independently selected from: halogen; —OR¹⁸; C₁₋₆ alkyl optionally substituted with one or more substituents independently selected from halogen, —OR¹⁸, and C₃₋₆ carbocycle; and C₃₋₁₀ carbocycle and 3- to 10-membered heterocycle, wherein the C₃₋₁₀ carbocycle and 3- to 10-membered heterocycle are each optionally substituted with one or more substituents independently selected from halogen and C₁₋₄alkyl. In some embodiments, B is selected from C₃₋₁₂ carbocycle and 3- to 12-membered heterocycle, any of which is optionally substituted with one or more substituents independently selected from: halogen; C₁₋₆ alkyl optionally substituted with one or more substituents independently selected from halogen, and C₃₋₆ carbocycle; and C₃₋₁₀ carbocycle and 3- to 10-membered heterocycle, wherein the C₃₋₁₀ carbocycle and 3- to 10-membered heterocycle are each optionally substituted with one or more substituents independently selected from halogen and C₁₋₄ alkyl.

In some embodiments, for the compound or salt of Formulas (Ia), (IIa) or (IIIa), R¹⁸ is independently selected at each occurrence from:
hydrogen;
C₁₋₆ alkyl optionally substituted with one or more substituents independently selected from: halogen, —OR²², —SR²², —N(R²)₂, —C(O)R²², —C(O)OR²², —OC(O)R²², —OC(O)N(R²²)₂, —C(O)N(R²)₂, —N(R²²)C(O)R², —NO₂, ═O, ═S, ═N(R²²), —N₃, —CN, C₃₋₁₀ carbocycle, and 3- to 10-membered heterocycle, wherein the C₃₋₁₀ carbocycle and 3- to 10-membered heterocycle each are optionally substituted with one or more substituents independently selected from halogen, C₁₋₆ alkyl, C₁₋₆ haloalkyl, —OR²², —SR²², and —N(R²²)₂; and C₃₋₁₀ carbocycle and 3- to 10-membered heterocycle, any of which is optionally substituted with one or more substituents independently selected from: halogen, C₁₋₆ alkyl, C₁₋₆ haloalkyl.

In some embodiments, for the compound or salt of Formulas (Ia), (IIa) or (IIIa), R¹⁸ is independently selected at each occurrence from: hydrogen; C₁₋₆ alkyl optionally substituted with one or more substituents independently selected from halogen and C₃₋₆ carbocycle; and C₃₋₁₀ carbocycle and 3- to 10-membered heterocycle, any of which is optionally substituted with one or more substituents independently selected from: halogen, C₁₋₆ alkyl, C₁₋₆ haloalkyl.

In some embodiments, for the compound or salt of Formulas (Ia), (IIa) or (IIIa), R²² is independently selected at each occurrence from:
hydrogen;
C₁₋₄ alkyl optionally substituted with one or more substituents independently selected from halogen, hydroxyl, C₃₋₆ carbocycle, and 3- to 6-membered heterocycle, wherein each C₆ carbocycle and 3- to 6-membered heterocycle is optionally substituted with one or more substituents independently selected from halogen C₁₋₄ alkyl, and —C(O)N(R²³)₂; and
C₃₋₆ carbocycle and 3- to 12-membered heterocycle, any of which is optionally substituted with one or more substituents independently selected from halogen, C₁₋₄ alkyl, C₁₋₄ haloalkyl, C₁₋₄ alkoxy, and ═O; and
R²³ is independently selected at each occurrence from hydrogen and C₁₋₄ alkyl.

In some embodiments, for the compound or salt of Formulas (Ia), (IIa) or (IIIa), R²² is independently selected at each occurrence from hydrogen, C₁₋₄ alkyl, C₆ carbocycle, and 3- to 6-membered heterocycle, wherein the C₃₋₆ carbocycle and 3- to 6-membered heterocycle are each optionally substituted with one or more substituents independently selected from C₁₋₄ alkyl and C₁₋₄ alkoxy; and R²³ is independently selected at each occurrence from hydrogen and C₁₋₄alkyl.

In some embodiments, for the compound or salt of Formulas (Ia), (IIa) or (IIIa), the C₃₋₁₀ carbocycle and 3- to 12-membered heterocycle of B is selected from phenyl; pyridinyl, naphthyl; 1,2,3,4-tetrahydronaphthalene; indane; 7-azaindole; indazole; and chromane; any of which is optionally substituted with one or more substituents.

In some embodiments, for the compound or salt of Formulas (Ia), (IIa) or (IIIa), the C₃₋₁₂ carbocycle and 3- to 12-membered heterocycle of B is selected from monocyclic C₃₋₆ carbocycle and 3- to 7-membered monocyclic heterocycle any of which is optionally substituted with one or more substituents. In some embodiments, B is selected from phenyl and pyridinyl, any of which is optionally substituted with one or more substituents.

In some embodiments, for the compound or salt of Formulas (Ia), (IIa) or (IIIa), B is selected from bicyclic C₆₋₁₂ carbocycle and bicyclic 6- to 12-membered bicyclic heterocycle, any of which is optionally substituted with one or more substituents. In some embodiments, B is selected from naphthyl; 1,2,3,4-tetrahydronaphthalene; indane; 7-azaindole; indazole; and chromane; any of which is optionally substituted with one or more substituents.

In some embodiments, for the compound or salt of Formulas (Ia), (IIa) or (IIIa), the one or more optional substituents on B is independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═N(R¹⁸), —N₃, and —CN;
C₁₋₆ alkyl optionally substituted with one or more substituents independently selected from:
   halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸. —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═N(R¹⁸), —N₃, —CN, C₃₋₆ carbocycle and 3- to 6-membered heterocycle,
      wherein the C₃₋₆ carbocycle and 3- to 6-membered heterocycle are each optionally substituted with one or more substituents independently selected from halogen, C₁₋₄ alkyl, C₁₋₄ haloalkyl, and ═O; and
C₃₋₁₀ carbocycle and 3- to 10-membered heterocycle, any of which is optionally substituted with one or more substituents independently selected from halogen, C₁₋₄ alkyl, C₁₋₄ haloalkyl, and ═O.

In some embodiments, for the compound or salt of Formulas (Ia), (IIa) or (IIIa), the one or more optional substituents on B is independently selected from:
halogen, —OR¹⁸, —N(R¹⁸)₂, —C(O)R¹⁸, ═O, and —CN;
C₁₋₆ alkyl optionally substituted with one or more substituents independently selected from halogen, —OR¹⁸, —SR¹⁸, —N(R¹⁸)₂, —C(O)R¹⁸, ═O, —CN, C₃₋₆ carbocycle, and 3- to 6-membered heterocycle, wherein the C₃₋₆ carbocycle and 3- to 6-membered heterocycle are each optionally substituted with one or more substituents independently selected from halogen, C₁₋₄ alkyl, C₁₋₄ haloalkyl, and ═O; and
C₃₋₁₀ carbocycle and 3- to 10-membered heterocycle, any of which is optionally substituted with one or more substituents independently selected from halogen, C₁₋₄ alkyl, C₁₋₄ haloalkyl, and ═O.

In some embodiments, for the compound or salt of Formulas (Ia), (IIa) or (IIIa), the one or more optional substituents on B is independently selected from
halogen and —OR¹⁸;
C₁₋₆ alkyl optionally substituted with one or more substituents independently selected from halogen, OR¹⁸, and C₃₋₆ carbocycle; and
C₃₋₁₀ carbocycle and 3- to 10-membered heterocycle, wherein the C₃₋₁₀ carbocycle and 3- to 10-membered heterocycle are each optionally substituted with one or more substituents independently selected from halogen and C₁₋₄ alkyl.

In some embodiments, for the compound or salt of Formulas (Ia), (IIa) or (IIIa), B is selected from C₃₋₁₂ carbocycle and 3- to 12-membered heterocycle, any of which is optionally substituted with one or more substituents independently selected from: halogen; —OR¹⁸; C₁₋₆ alkyl optionally substituted with one or more substituents independently selected from halogen, —OR¹⁸, and C₃₋₆ carbocycle; and C₃₋₁₀ carbocycle and 3- to 10-membered heterocycle, wherein the C₃₋₁₀ carbocycle and 3- to 10-membered heterocycle are each optionally substituted with one or more substituents independently selected from halogen and C₁₋₄ alkyl; wherein R¹⁸ is independently selected at each occurrence from: hydrogen, C₁₋₆ alkyl, and C₃₋₁₀ carbocycle optionally substituted with one or more substituents independently selected from halogen, C₁₋₆ alkyl, C₁₋₆ haloalkyl.

In some embodiments, for the compound or salt of for the compound or salt of Formulas (Ia), (IIa) or (IIIa), B is selected from C₃₋₁₂ carbocycle and 3- to 12-membered heterocycle, any of which is optionally substituted with one or more substituents independently selected from: halogen, trifluoromethyl, cyclopropyl, phenyl,

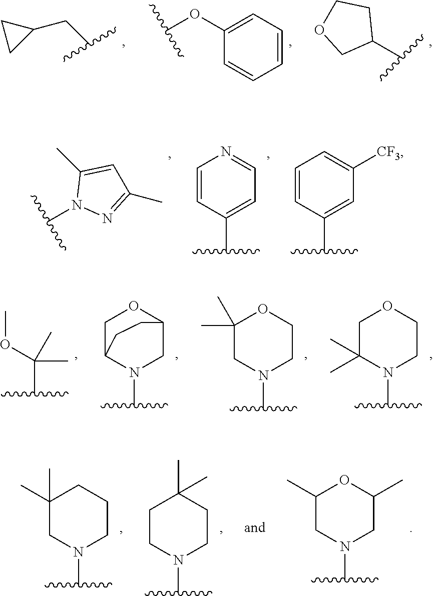

In some embodiments, for the compound or salt of Formulas (Ia), (IIa) or (IIIa), B is selected from C₃₋₁₂ carbocycle and 3- to 12-membered heterocycle, any of which is optionally substituted with one or more substituents independently selected from: halogen, trifluoromethyl, cyclopropyl, phenyl,

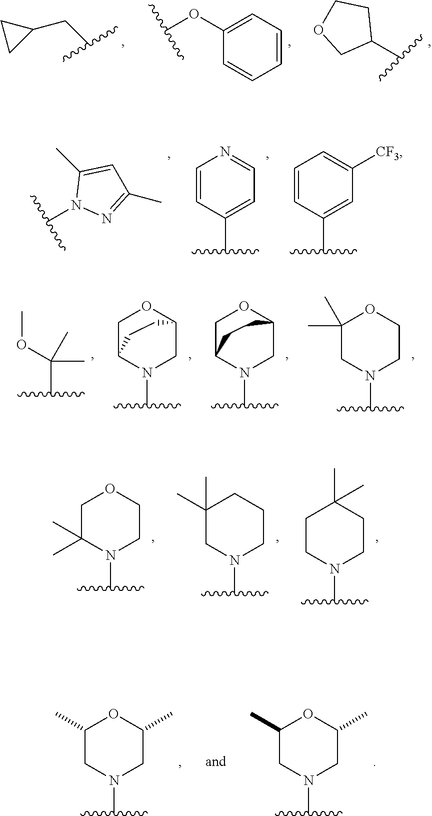

In some embodiments, for the compound or salt of Formulas (Ia), (IIa) or (IIIa), B is selected from phenyl; pyridinyl, naphthyl; 1,2,3,4-tetrahydronaphthalene; indane; 7-azaindole; indazole; and chromane; any of which is optionally substituted with one or more substituents independently selected from:
halogen, —OR¹⁸, —N(R¹⁸)₂, —C(O)R¹⁸, and ═O;
C₁₋₆ alkyl optionally substituted with one or more substituents independently selected from halogen, —OR¹⁸, —N(R¹⁸)₂, ═O, —CN, C₃₋₄ carbocycle, and 3- to 6-membered heterocycle, wherein the C₃₋₆ carbocycle and 3- to 6-membered heterocycle are each optionally substituted with one or more substituents independently selected from halogen, C₁₋₄ alkyl, C₁₋₄ haloalkyl, and ═O; and
C₃₋₁₀ carbocycle and 3- to 10-membered heterocycle, any of which is optionally substituted with one or more substituents independently selected from halogen, C₁₋₄ alkyl, C₁₋₄haloalkyl, and ═O.

In some embodiments, for the compound or salt of Formulas (Ia), (IIa) or (IIIa), B is selected from phenyl; pyridinyl, naphthyl; 1,2,3,4-tetrahydronaphthalene; indane; 7-azaindole; indazole; and chromane; any of which is optionally substituted with one or more substituents independently selected from: halogen; —OR¹⁸; C₁₋₆ alkyl optionally substituted with one or more substituents independently selected from halogen, —OR¹⁸, and C₃₋₆ carbocycle; and C₃₋₁₀ carbocycle and 3- to 10-membered heterocycle, wherein the C₃₋₁₀ carbocycle and 3- to 10-membered heterocycle are each optionally substituted with one or more substituents independently selected from halogen and C₁₋₄alkyl.

In some embodiments, for the compound or salt of Formulas (Ia), (IIa) or (IIIa), B is selected from phenyl; pyridinyl, naphthyl; 1,2,3,4-tetrahydronaphthalene; indane; 7-azaindole; indazole; and chromane; any of which is optionally substituted with one or more substituents independently selected from: halogen; C₁₋₆ alkyl optionally substituted with one or more substituents independently selected from halogen, and C₃₋₆ carbocycle; and C₃₋₁₀ carbocycle and 3 to 10-membered heterocycle, wherein the C₃₋₁₀ carbocycle and 3- to 10-membered heterocycle are each optionally substituted with one or more substituents independently selected from halogen and C₁₋₄ alkyl.

In some embodiments, for the compound or salt of Formulas (Ia), (IIa) or (IIIa), B is selected from phenyl; pyridinyl, naphthyl; 1,2,3,4-tetrahydronaphthalene; indane; 7-azaindole; indazole; and chromane; any of which is optionally substituted with one or more substituents independently selected from: halogen; —OR¹⁸; C₁₋₆ alkyl optionally substituted with one or more substituents independently selected from halogen, —OR¹⁸, and C₃₋₁₀ carbocycle; and C₃₋₁₀ carbocycle and 3- to 10-membered heterocycle, wherein the C₃₋₁₀ carbocycle and 3- to 10-membered heterocycle are each optionally substituted with one or more substituents independently selected from halogen and C₁₋₄alkyl; wherein R¹⁸ is independently selected at each occurrence from: hydrogen, C₁₋₆ alkyl, and C₃₋₁₀ carbocycle optionally substituted with one or more substituents independently selected from halogen, C₁₋₆ alkyl, C₁₋₆ haloalkyl.

In some embodiments, for the compound or salt of Formulas (Ia), (IIa) or (IIIa), B is selected from phenyl; pyridinyl, naphthyl; 1,2,3,4-tetrahydronaphthalene; indane; 7-azaindole; indazole; and chromane; any of which is optionally substituted with one or more substituents independently selected from: halogen, trifluoromethyl, cyclopropyl, phenyl;

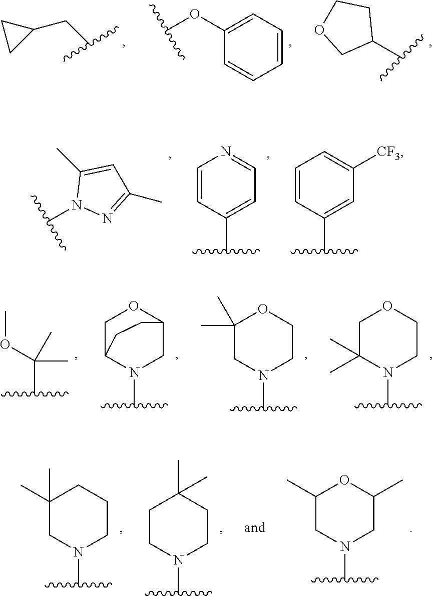

In some embodiments, for the compound or salt of Formulas (Ia), (IIa) or (IIIa), B is selected from phenyl; pyridinyl, naphthyl; 1,2,3,4-tetrahydronaphthalene; indane; 7-azaindole; indazole; and chromane; any of which is optionally substituted with one or more substituents independently selected from: halogen, trifluoromethyl, cyclopropyl, phenyl,

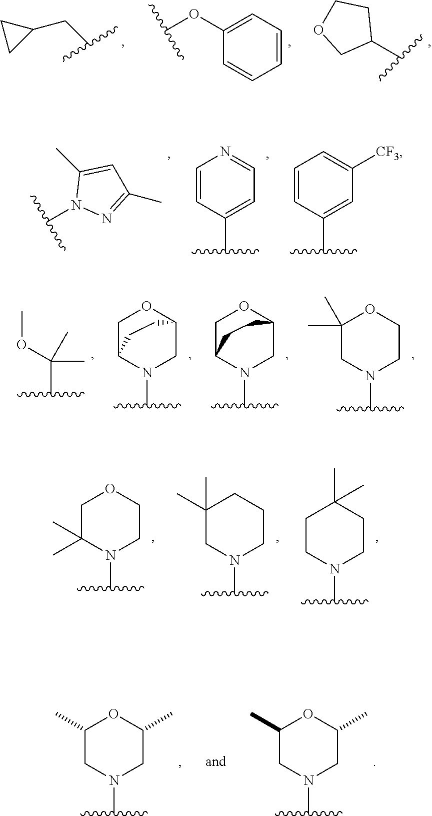

In some embodiments, for the compound or salt of Formulas (Ia), (IIa) or (IIIa), the C₃₋₁₂ carbocycle and 3- to 12-membered heterocycle of B is selected from monocyclic C₃₋₆ carbocycle and 3- to 7-membered monocyclic heterocycle, any of which is optionally substituted with one or more substituents independently selected from:
halogen, —OR¹⁸, —N(R¹⁸)₂, —C(O)R¹⁸, and ═O;
C₁₋₆ alkyl optionally substituted with one or more substituents independently selected from halogen, —OR¹⁸, —N(R¹⁸)₂, ═O, —CN, C₃₋₆ carbocycle, and 3- to 6-membered heterocycle, wherein the C₃₋₆ carbocycle and 3- to 6-membered heterocycle are each optionally substituted with one or more substituents independently selected from halogen, C₁₋₄ alkyl, C₁₋₄ haloalkyl, and ═O; and C₃₋₁₀ carbocycle and 3- to 10-membered heterocycle, any of which is optionally substituted with one or more substituents independently selected from halogen, C₁₋₄ alkyl, C₁₋₄ haloalkyl, and ═O.

In some embodiments, for the compound or salt of Formulas (Ia), (IIa) or (IIa), the C₃₋₁₀ carbocycle and 3- to 12-membered heterocycle of B is selected from monocyclic C₃₋₆ carbocycle and 3- to 7-membered monocyclic heterocycle, any of which is optionally substituted with one or more substituents independently selected from: halogen; —OR¹⁸; C₁₋₆ alkyl optionally substituted with one or more substituents independently selected from halogen, —OR¹⁸, and C₃₋₆ carbocycle; and C₃₋₁₀ carbocycle and 3- to 10-membered heterocycle, wherein the C₃₋₁₀ carbocycle and 3- to 10-membered heterocycle are each optionally substituted with one or more substituents independently selected from halogen and C₁₋₄ alkyl.

In some embodiments, for the compound or salt of Formulas (Ia), (IIa) or (IIIa), the C₃₋₁₂ carbocycle and 3- to 12-membered heterocycle of B is selected from monocyclic C₃₋₆ carbocycle and 3- to 7-membered monocyclic heterocycle, any of which is optionally substituted with one or more substituents independently selected from halogen; C₁₋₆alkyl optionally substituted with one or more substituents independently selected from halogen, and C₃₋₆ carbocycle; and C₃₋₁₀ carbocycle and 3- to 10-membered heterocycle, wherein the C₃₋₁₀ carbocycle and 3- to 10-membered heterocycle are each optionally substituted with one or more substituents independently selected from halogen and C₁₋₄ alkyl.

In some embodiments, for the compound or salt of Formulas (Ia), (IIa) or (IIIa), the C₃₋₁₂ carbocycle and 3- to 12-membered heterocycle of B is selected from monocyclic C₃₋₆ carbocycle and 3- to 7-membered monocyclic heterocycle, any of which is optionally substituted with one or more substituents independently selected from: halogen; —OR¹⁸; C₁₋₄ alkyl optionally substituted with one or more substituents independently selected from halogen, —OR¹⁸, and C₃₋₆ carbocycle; and C₃₋₁₀ carbocycle and 3- to 10-membered heterocycle, wherein the C₃₋₁₀ carbocycle and 3- to 10-membered heterocycle are each optionally substituted with one or more substituents independently selected from halogen and C₁₋₄ alkyl; wherein R¹⁸ is independently selected at each occurrence from: hydrogen, C₁₋₆ alkyl, and C₃₋₁₀ carbocycle optionally substituted with one or more substituents independently selected from halogen, C₁₋₆alkyl, C₁₋₆ haloalkyl.

In some embodiments, for the compound or salt of Formulas (Ia), (IIa) or (IIIa), the C₃₋₁₂ carbocycle and 3- to 12-membered heterocycle of B is selected from monocyclic C₃₋₆ carbocycle and 3- to 7-membered monocyclic heterocycle, any of which is optionally substituted with one or more substituents independently selected from: halogen, trifluoromethyl, cyclopropyl, phenyl,

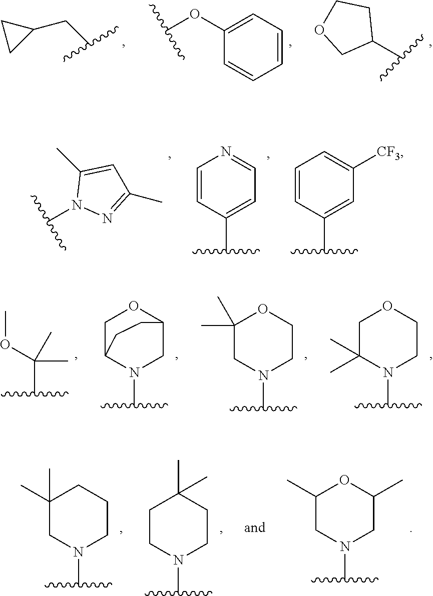

In some embodiments, for the compound or salt of Formulas (Ia), (IIa) or (IIIa), B is selected from phenyl and pyridinyl, any of which is optionally substituted with one or more substituents independently selected from:
halogen, —OR¹⁸, —N(R¹⁸)₂, —C(O)R¹⁸, and ═O;
C₁₋₆ alkyl optionally substituted with one or more substituents independently selected from halogen, —OR¹⁸, —N(R¹⁸)₂, ═O, —CN, C₃₋₆ carbocycle, and 3- to 6-membered heterocycle, wherein the C₃₋₆ carbocycle and 3- to 6-membered heterocycle are each optionally substituted with one or more substituents independently selected from halogen, C₁₋₄ alkyl, C₁₋₄ haloalkyl, and ═O; and C₃₋₁₀ carbocycle and 3- to 10-membered heterocycle, any of which is optionally substituted with one or more substituents independently selected from halogen, C₁₋₄ alkyl, C₁₋₄ haloalkyl, and ═O.

In some embodiments, for the compound or salt of Formulas (Ia), (IIa) or (IIIa), B is selected from phenyl and pyridinyl, any of which is optionally substituted with one or more substituents independently selected from: halogen; —OR¹⁸; C₁₋₆, alkyl optionally substituted with one or more substituents independently selected from halogen, —OR¹⁸, and C₃₋₆ carbocycle; and C₃₋₁₀ carbocycle and 3- to 10-membered heterocycle, wherein the C₃₋₁₀ carbocycle and 3- to 10-membered heterocycle are each optionally substituted with one or more substituents independently selected from halogen and C₁₋₄ alkyl.

In some embodiments, for the compound or salt of Formulas (Ia), (IIa) or (IIIa), B is selected from phenyl and pyridinyl, any of which is optionally substituted with one or more substituents independently selected from halogen; C₁₋₆ alkyl optionally substituted with one or more substituents independently selected from halogen, and C₃₋₆ carbocycle; and C₃₋₁₀ carbocycle and 3 to 10-membered heterocycle, wherein the C₃₋₁₀, carbocycle and 3- to 10-membered heterocycle are each optionally substituted with one or more substituents independently selected from halogen and C₁₋₄ alkyl.

In some embodiments, for the compound or salt of Formulas (Ia), (IIa) or (IIIa), B is selected from phenyl and pyridinyl, any of which is optionally substituted with one or more substituents independently selected from: halogen; —OR¹⁸; C₁₋₆ alkyl optionally substituted with one or more substituents independently selected from halogen, —OR¹⁸, and C₃₋₆ carbocycle; and C₃₋₁₀ carbocycle and 3- to 10-membered heterocycle, wherein the C₃₋₁₀ carbocycle and 3- to 10-membered heterocycle are each optionally substituted with one or more substituents independently selected from halogen and C₁₋₄ alkyl; wherein R¹⁸ is independently selected at each occurrence from: hydrogen, C₁₋₆ alkyl, and C₃₋₁₀ carbocycle optionally substituted with one or more substituents independently selected from halogen, C₁₋₆ alkyl, C₁₋₆haloalkyl.

In some embodiments, for the compound or salt of Formulas (Ia), (IIa) or (IIIa), B is selected from phenyl and pyridinyl, any of which is optionally substituted with one or more substituents independently selected from: halogen, trifluoromethyl, cyclopropyl, phenyl,

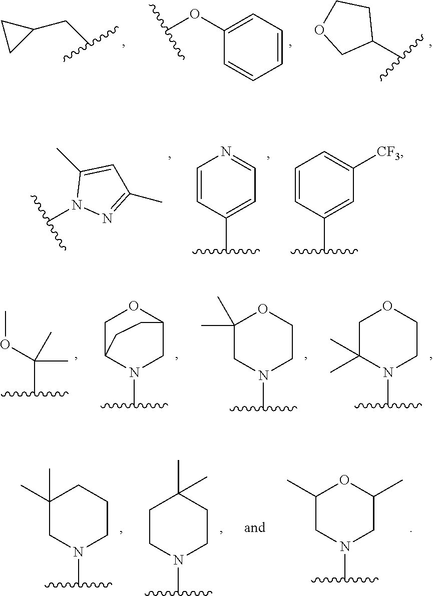

In some embodiments, for the compound or salt of Formulas (Ia), (IIa) or (IIIa), B is selected from bicyclic C₆₋₂ carbocycle and bicyclic 6- to 12-membered bicyclic heterocycle, any of which is optionally substituted with one or more substituents independently selected from:
halogen, —OR¹⁸, —N(R¹⁸)₂, —C(O)R¹⁸, and ═O;
C₁₋₆ alkyl optionally substituted with one or more substituents independently selected from halogen, —OR¹⁸, —N(R¹⁸)₂, ═O, —CN, C₃₋₆ carbocycle, and 3- to 6-membered heterocycle, wherein the C₃₋₄ carbocycle and 3- to 6-membered heterocycle are each optionally substituted with one or more substituents independently selected from halogen, C₁₋₄ alkyl, C₁₋₄ haloalkyl, and ═O; and
C₃₋₁₀ carbocycle and 3- to 10-membered heterocycle, any of which is optionally substituted with one or more substituents independently selected from halogen, C₁₋₄ alkyl, C₁₋₄haloalkyl, and ═O.

In some embodiments, for the compound or salt of Formulas (Ia), (IIa) or (IIIa), B is selected from bicyclic C₆₋₁₂ carbocycle and bicyclic 6- to 12-membered bicyclic heterocycle, any of which is optionally substituted with one or more substituents independently selected from: halogen; —OR¹⁸; C₁₋₆ alkyl optionally substituted with one or more substituents independently selected from halogen, —OR¹⁸, and C₃₋₆ carbocycle; and C₃₋₁₀ carbocycle and 3- to 10-membered heterocycle, wherein the C₃₋₁₀ carbocycle and 3- to 10-membered heterocycle are each optionally substituted with one or more substituents independently selected from halogen and C₁₋₄ alkyl.

In some embodiments, for the compound or salt of Formulas (Ia), (IIa) or (IIIa), B is selected from bicyclic C₆₋₁₂ carbocycle and bicyclic 6- to 12-membered bicyclic heterocycle, any of which is optionally substituted with one or more substituents independently selected from halogen; C₁₋₆ alkyl optionally substituted with one or more substituents independently selected from halogen, and C₃₋₆ carbocycle; and C₃₋₁₀ carbocycle and 3- to 10-membered heterocycle, wherein the C₃₋₁₀ carbocycle and 3- to 10-membered heterocycle are each optionally substituted with one or more substituents independently selected from halogen and C₁₋₄ alkyl.

In some embodiments, for the compound or salt of Formulas (Ia), (IIa) or (IIIa), B is selected from bicyclic C₆₋₁₂ carbocycle and bicyclic 6- to 12-membered bicyclic heterocycle, any of which is optionally substituted with one or more substituents independently selected from: halogen; —OR¹⁸; C₁₋₆ alkyl optionally substituted with one or more substituents independently selected from halogen, —OR¹⁸, and C₃₋₆ carbocycle; and C₃₋₁₀ carbocycle and 3- to 10-membered heterocycle, wherein the C₃₋₁₀ carbocycle and 3- to 10-membered heterocycle are each optionally substituted with one or more substituents independently selected from halogen and C₁₋₄ alkyl; wherein R¹⁸ is independently selected at each occurrence from: hydrogen, C₁₋₆ alkyl, and C₃₋₁₀ carbocycle optionally substituted with one or more substituents independently selected from halogen, C₁₋₆ alkyl, C₁₋₆ haloalkyl.

In some embodiments, for the compound or salt of Formulas (Ia), (IIa) or (IIIa), B is selected from bicyclic C₆₋₁₂ carbocycle and bicyclic 6- to 12-membered bicyclic heterocycle, any of which is optionally substituted with one or more substituents independently selected from: halogen, trifluoromethyl, cyclopropyl, phenyl,

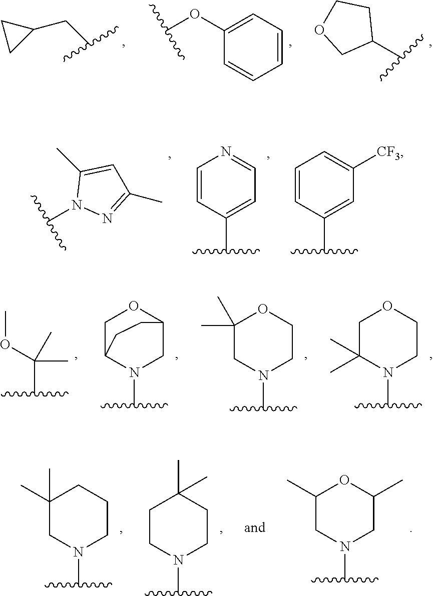

In some embodiments, for the compound or salt of Formulas (Ia), (IIa) or (IIIa), B is selected from naphthyl; 1,2,3,4-tetrahydronaphthalene; indane; 7-azaindole; indazole; and chromane; any of which is optionally substituted with one or more substituents independently selected from:
halogen, —OR¹⁸, —N(R¹⁸)₂, —C(O)R¹⁸, and ═O;
C₁₋₆ alkyl optionally substituted with one or more substituents independently selected from halogen, —OR¹⁸, —N(R¹⁸)₂, ═O, —CN, C₃₋₆ carbocycle, and 3- to 6-membered heterocycle, wherein the C₃₋₆ carbocycle and 3- to 6-membered heterocycle are each optionally substituted with one or more substituents independently selected from halogen, C₁₋₄ alkyl, C₁₋₄ haloalkyl, and ═O; and
C₃₋₁₀ carbocycle and 3- to 10-membered heterocycle, any of which is optionally substituted with one or more substituents independently selected from halogen, C₁₋₄ alkyl, C₁₋₄ haloalkyl, and ═O.

In some embodiments, for the compound or salt of Formulas (Ia), (IIa) or (IIIa), B is selected from naphthyl; 1,2,3,4-tetrahydronaphthalene; indane; 7-azaindole; indazole; and chromane; any of which is optionally substituted with one or more substituents independently selected from: halogen; —OR¹⁸; C₁₋₆ alkyl optionally substituted with one or more substituents independently selected from halogen, —OR¹⁸, and C₃₋₆ carbocycle; and C₃₋₁₀ carbocycle and 3- to 10-membered heterocycle, wherein the C₃₋₁₀ carbocycle and 3- to 10-membered heterocycle are each optionally substituted with one or more substituents independently selected from halogen and C₁₋₄ alkyl.

In some embodiments, for the compound or salt of Formulas (Ia), (IIa) or (IIa), B is selected from naphthyl; 1,2,3,4-tetrahydronaphthalene; indane; 7-azaindole; indazole; and chromane; any of which is optionally substituted with one or more substituents independently selected from halogen, and C₃₋₆ carbocycle; and C₃₋₁₀ carbocycle and 3- to 10-membered heterocycle, wherein the C₃₋₁₀ carbocycle and 3- to 10-membered heterocycle are each optionally substituted with one or more substituents independently selected from halogen and C₁₋₄alkyl.

In some embodiments, for the compound or salt of Formulas (Ia), (IIa) or (IIIa), B is selected from naphthyl; 1,2,3,4-tetrahydronaphthalene; indane; 7-azaindole; indazole; and chromane; any of which is optionally substituted with one or more substituents independently selected from halogen, —OR¹⁸, and C₃₋₁₀ carbocycle; and C₃₋₁₀ carbocycle and 3- to 10-membered heterocycle, wherein the C₃₋₁₀ carbocycle and 3- to 10-membered heterocycle are each optionally substituted with one or more substituents independently selected from halogen and C₁₋₄ alkyl; wherein R¹⁸ is independently selected at each occurrence from: hydrogen, C₁₋₆ alkyl, and C₃₋₁₀ carbocycle optionally substituted with one or more substituents independently selected from halogen, C₁₋₆ alkyl, C₁₋₆ haloalkyl.

In some embodiments, for the compound or salt of Formulas (Ia), (IIa) or (IIIa), B is selected from naphthyl; 1,2,3,4-tetrahydronaphthalene; indane; 7-azaindole; indazole; and chromane; any of which is optionally substituted with one or more substituents independently selected from: halogen, trifluoromethyl, cyclopropyl, phenyl,

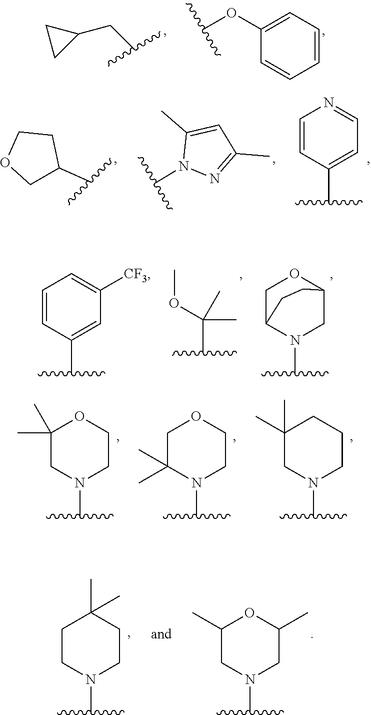

In some embodiments, for the compound or salt of Formulas (Ia), (IIa) or (IIIa), B is selected from:

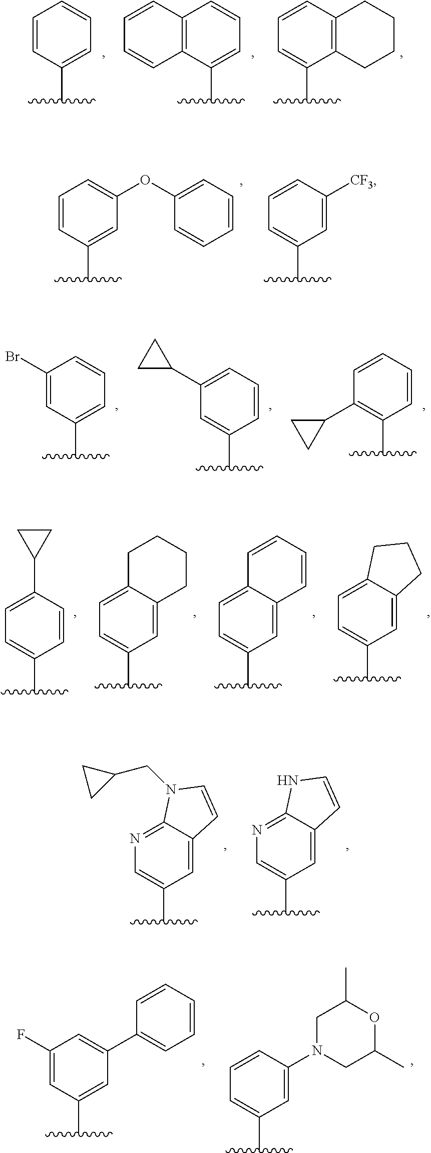

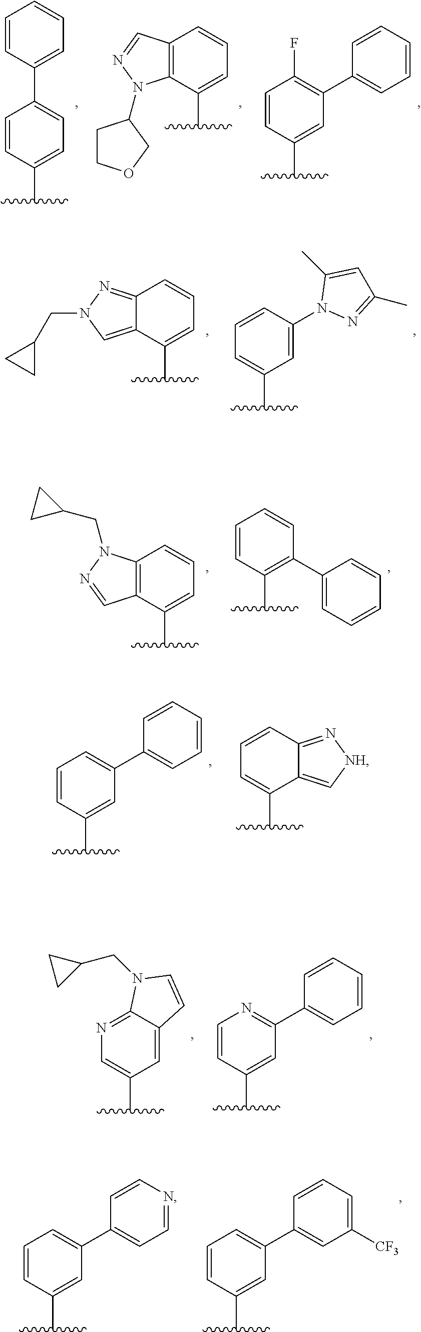

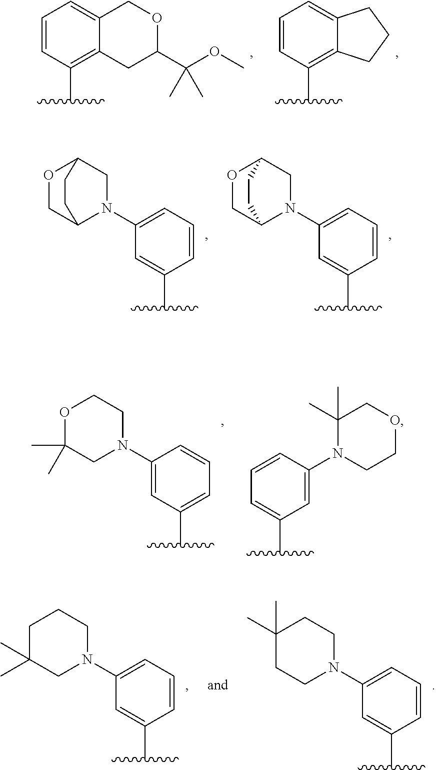

In some embodiments, for the compound or salt of Formulas (Ia), (IIa) or (IIIa), B is selected from

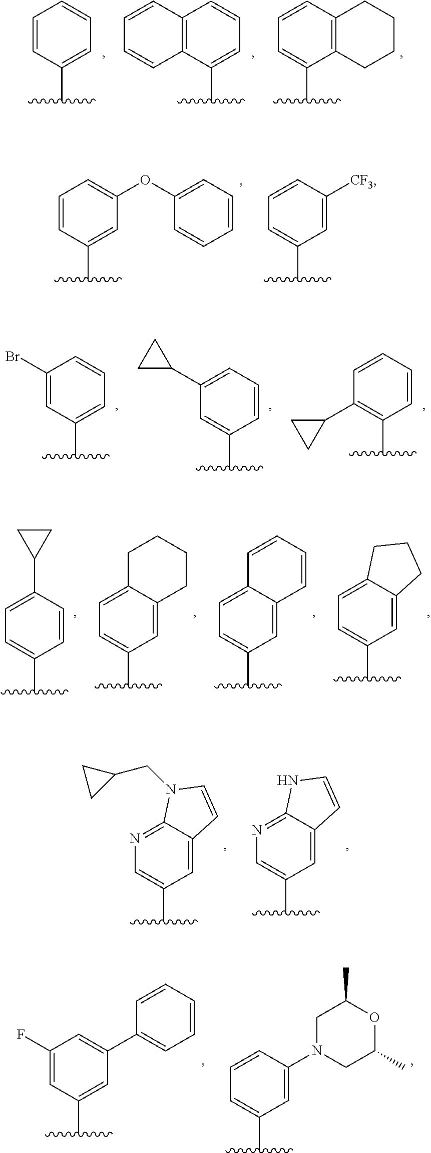

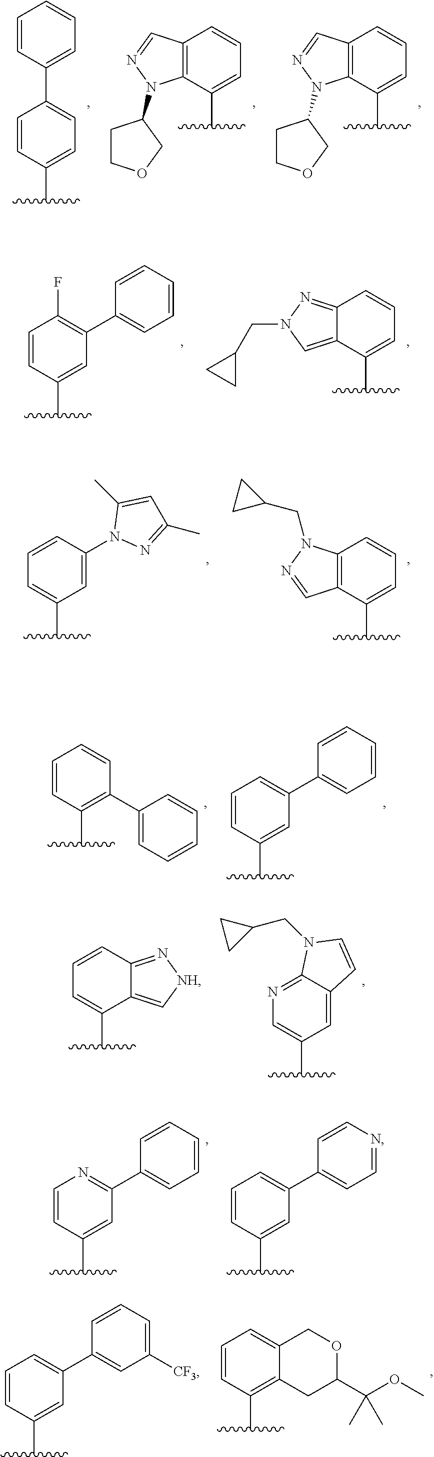

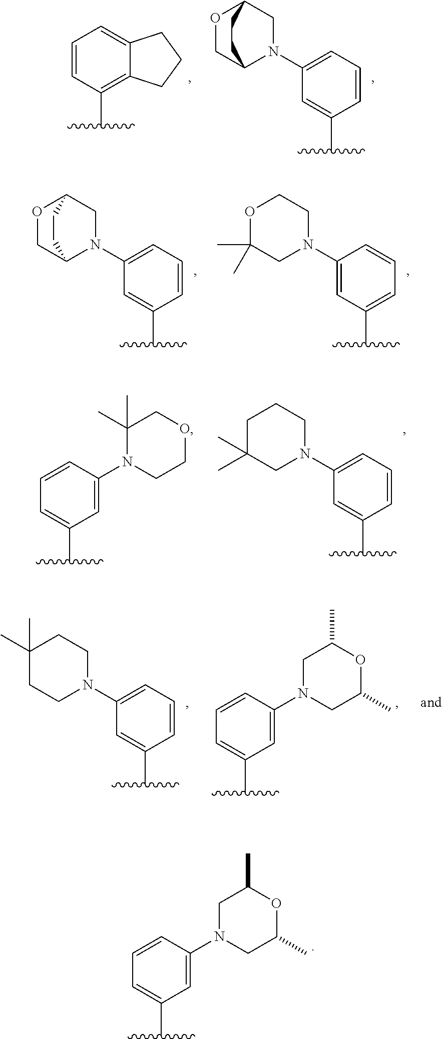

In some embodiments, for the compound or salt of Formulas (Ia), (IIa) or (IIIa), B is selected from —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹³)S(O)₂(R¹⁸), —S(O)R¹⁸, —S(O)₂R¹⁸, and —S(O)₂N(R¹³)₂. In some embodiments, B is selected from —OR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, and —N(R¹⁸)S(O)₂(R¹⁸). In some embodiments, B is selected from —OR¹⁸, —N(R¹⁸)₂, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, and —N(R¹⁸)S(O)₂(R¹⁸). In some embodiments, B is selected from —OR¹⁸, —N(R¹⁸)₂, —OC(O)R¹⁸, —OC(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, and —N(R¹⁸)S(O)₂(R¹⁸).

In some embodiments, for the compound or salt of Formulas (Ia), (IIa) or (IIIa), B is selected from —OR¹⁸, —N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, and —N(R¹⁸)S(O)₂(R¹⁸). In some embodiments, B is selected from —N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, and —N(R¹⁸)S(O)₂(R¹⁸). In some embodiments, B is selected from —N(R¹⁸)₂, —N(R¹⁸)C(O)R¹⁸, —N(R¹⁸)C(O)OR¹⁸, —N(R¹⁸)C(O)N(R¹⁸)₂, and —N(R¹⁸)S(O)₂(R¹⁸). In some embodiments, B is selected from —N(R¹⁸)₂, —N(R¹⁸)C(O)R¹⁸, —N(R¹⁸)C(O)N(R¹⁸)₂, and —N(R¹⁸)S(O)₂(R¹⁸). In some embodiments, B is —N(R¹⁸)₂. In some embodiments, B is —N(R¹⁸)C(O)R¹⁸. In some embodiments, B is —N(R¹⁸)C(O)N(R¹⁸)₂. In some embodiments. B is —N(R¹⁸)S(O)₂(R¹⁸).

In some embodiments, for the compound or salt of Formulas (Ia), (IIa) or (IIIa), R¹⁸ is independently selected at each occurrence from:
hydrogen;
C₁₋₆ alkyl optionally substituted with one or more substituents independently selected from.
   halogen, —OR²², —SR²², —N(R²²)₂, —C(O)R²², —C(O)OR²², —OC(O)R²², —OC(O)N(R²²)₂, —C(O)N(R²)₂, —N(R²²)C(O)R²², —NO₂, ═O, ═S, ═N(R²²), —N₃, —CN, C₃₋₁₀ carbocycle, and 3- to 10-membered heterocycle,
      wherein the C₃₋₁₀ carbocycle and 3- to 10-membered heterocycle each are optionally substituted with one or more substituents independently selected from halogen, C₁₋₆ alkyl, C₁₋₄ haloalkyl, —OR²², —SR²², and —N(R²²)₂; and
C₃₋₁₀ carbocycle and 3- to 10-membered heterocycle, any of which is optionally substituted with one or more substituents independently selected from:
   halogen, C₁₋₆ alkyl, C₁₋₆ haloalkyl, —OR²², —SR²², —N(R²²)₂, —C(O)R²², —C(O)OR²², —OC(O)R², —OC(O)N(R²)₂, —C(O)N(R²)₂, —N(R²²)C(O)R²², —N(R²²)C(O)OR²², —N(R²²)C(O)N(R²²)₂, —N(R²²)C(S)N(R²²)₂, —N(R²²)S(O)₂(R²²), —S(O)R²², —S(O)₂R²², —S(O)₂N(R²²)₂, —NO₂, ═O, ═S, ═N(R²²), —N₃, —CN, C₃₋₆ carbocycle, and 3- to 6-membered heterocycle;
      wherein the C₃₋₆ carbocycle and 3- to 6-membered heterocycle are each optionally substituted with one or more substituents independently selected from halogen, C₁₋₄ alkyl, and C₁₋₄ haloalkyl.

In some embodiments, for the compound or salt of Formulas (Ia), (IIa) or (IIIa), R¹⁸ is independently selected at each occurrence from: hydrogen and C₁₋₆ alkyl optionally substituted with one or more substituents independently selected from:
halogen, —OR²², —SR²², —N(R²²)₂, —C(O)R²², —C(O)OR²², —OC(O)R²¹, —OC(O)N(R²²)₂, —C(O)N(R²)₂, —N(R²²)C(O)R²², —NO₂, ═O, ═S, ═N(R²²), —N₃, —CN, C₃₋₁₀ carbocycle, and 3- to 10-membered heterocycle,
   wherein the C₃₋₁₀ carbocycle and 3- to 10-membered heterocycle each are optionally substituted with one or more substituents independently selected from halogen, C₁₋₆ alkyl, C₁₋₆ haloalkyl, —OR²², —SR², and —N(R²)₂.

In some embodiments, for the compound or salt of Formulas (Ia), (IIa) or (IIIa), R¹⁸ is independently selected at each occurrence from: hydrogen and C₁₋₆alkyl optionally substituted with one or more substituents independently selected from: halogen, —OR²², —N(R²²)₂, ═O, —CN, C₃₋₁₀ carbocycle, and 3- to 10-membered heterocycle; wherein the C₃₋₁₀ carbocycle and 3- to 10-membered heterocycle each are optionally substituted with one or more substituents independently selected from halogen and C₁₋₆ alkyl.

In some embodiments, for the compound or salt of Formulas (Ia), (IIa) or (IIIa), R²² is independently selected at each occurrence from:
hydrogen;
C₁₋₄ alkyl optionally substituted with one or more substituents independently selected from halogen, hydroxyl, C₃₋₆ carbocycle, and 3- to 6-membered heterocycle, wherein each C₃₋₆ carbocycle and 3- to 6-membered heterocycle is optionally substituted with one or more substituents independently selected from halogen C₁₋₄ alkyl, and —C(O)N(R²³)₂; and
C₃₋₆ carbocycle and 3- to 12-membered heterocycle, any of which is optionally substituted with one or more substituents independently selected from halogen, C₁₋₄ alkyl, C₁₋₄ haloalkyl, C₁₋₄ alkoxy, and ═O; and
R²³ is independently selected at each occurrence from hydrogen and C₁₋₄ alkyl.

In some embodiments, for the compound or salt of Formulas (Ia), (IIa) or (IIIa), R²² is independently selected at each occurrence from hydrogen, C₁₋₄ alkyl, C₃₋₆ carbocycle, and 3- to 6-membered heterocycle, wherein the C₃₋₆ carbocycle and 3- to 6-membered heterocycle are each optionally substituted with one or more substituents independently selected from C₁₋₄ alkyl and C₁₋₄ alkoxy; and R²³ is independently selected at each occurrence from hydrogen and C₁₋₄ alkyl.

In some embodiments, for the compound or salt of Formulas (Ia), (IIa) or (IIIa), R¹⁸ is independently selected at each occurrence from: hydrogen, C₃₋₁₀ carbocycle and 3- to 10-membered heterocycle, wherein each C₃₋₁₀ carbocycle, and 3- to 10-membered heterocycle of R¹⁸ is optionally substituted with one or more substituents. In some embodiments, each C₃₋₁₀ carbocycle and 3- to 10-membered heterocycle of R¹⁸ is independently selected at each occurrence from hydrogen; and pyrrolidine, piperidine, phenyl, indoline, bicyclo[2.2.2]octane, cyclohexane, tetrahydropyran, pyridine, oxadiazole, pyrimidine, quinazoline, naphthalene, quinoline, thieno[3,2-d]pyrimidine, thieno[2,3-d]pyrimidine, benzothiazole, indane, thieno[2,3-d]pyrimidine oxide, and cyclopropyl, any of which is optionally substituted with one or more substituents.

In some embodiments, for the compound or salt of Formulas (Ia), (IIa) or (IIIa), each C₃₋₁₀ carbocycle and 3- to 10-membered heterocycle of R¹⁸ is independently selected at each occurrence from monocyclic C₃₋₆ carbocycle and 3- to 7-membered monocyclic heterocycle any of which is optionally substituted with one or more substituents. In some embodiments, each C₃₋₁₀ carbocycle and 3- to 10-membered heterocycle of R¹⁸ is independently selected at each occurrence from pyrrolidine, piperidine, phenyl, cyclohexane, tetrahydropyran, pyridine, oxadiazole, pyrimidine, and cyclopropyl, any of which is optionally substituted with one or more substituents. In some embodiments, R¹⁸ is independently selected at each occurrence from hydrogen; and pyrrolidine, piperidine, phenyl, cyclohexane, tetrahydropyran, pyridine, oxadiazole, pyrimidine, and cyclopropyl, any of which is optionally substituted with one or more substituents.

In some embodiments, for the compound or salt of Formulas (Ia), (IIa) or (IIIa), each C₃₋₁₀ carbocycle and 3- to 10-membered heterocycle of R¹⁸ is independently selected at each occurrence from bicyclic C₆₋₁₀ carbocycle and bicyclic 6- to 10-membered bicyclic heterocycle, any of which is optionally substituted with one or more substituents. In some embodiments, each C₃₋₁₀ carbocycle and 3- to 10-membered heterocycle of R¹⁸ is independently selected at each occurrence from indoline, bicyclo[2.2.2]octane, quinazoline, naphthalene, quinoline, thieno[3,2-d]pyrimidine, thieno[2,3-d]pyrimidine, benzothiazole, indane, and thieno[2,3-d]pyrimidine oxide, any of which is optionally substituted with one or more substituents. In some embodiments, R¹⁸ is independently selected at each occurrence from hydrogen; and indoline, bicyclo[2.2.2]octane, quinazoline, naphthalene, quinoline, thieno[3,2-d]pyrimidine, thieno[2,3-d]pyrimidine, benzothiazole, indane, and thieno[2,3-d]pyrimidine oxide, any of which is optionally substituted with one or more substituents.

In some embodiments, for the compound or salt of Formulas (Ia), (IIa) or (IIIa), the C₃₋₁₀ carbocycle and 3- to 10-membered heterocycle of R¹⁸ are each optionally substituted at each occurrence with one or more substituents independently selected from: halogen, C₁₋₆ alkyl, C₁₋₆ haloalkyl, —OR²², —N(R²²)₂, —C(O)R²², —C(O)N(R²²)₂, —N(R²²)C(O)R²², —S(O)R²², ═O, —CN, C₃₋₆ carbocycle, and 3- to 6-membered heterocycle, wherein the C₃₋₆ carbocycle and 3- to 6-membered heterocycle are each optionally substituted with one or more substituents independently selected from halogen, C₁₋₄ alkyl, and C₁₋₄ haloalkyl. In some embodiments, the C₃₋₁₀ carbocycle and 3- to 10-membered heterocycle of R¹⁸ are each optionally substituted at each occurrence with one or more substituents independently selected from halogen, C₁₋₆alkyl, C₁₋₆haloalkyl, —N(R²²)₂, —C(O)R²², —C(O)N(R²²)₂, —S(O)₂R²², ═O, C₃₋₆ carbocycle, and 3- to 6-membered heterocycle, wherein the C₃₋₆ carbocycle and 3- to 6-membered heterocycle are each optionally substituted with one or more substituents independently selected from halogen and C₁₋₄haloalkyl.

In some embodiments, for the compound or salt of Formulas (Ia), (IIa) or (IIIa), the C₃₋₁₀ carbocycle and 3- to 10-membered heterocycle of R¹⁸ are each optionally substituted at each occurrence with one or more substituents independently selected from: halogen, methyl, trifluoromethyl, cyclopropyl, phenyl, —NH₂, ═O,

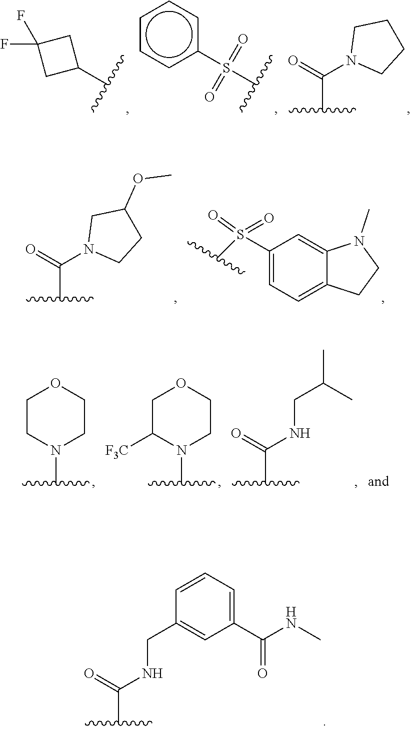

In some embodiments, the C₃₋₁₀ carbocycle and 3- to 10-membered heterocycle of R¹¹ are each optionally substituted at each occurrence with one or more substituents independently selected from: halogen, methyl, trifluoromethyl, cyclopropyl, phenyl, —NH₂, ═O,

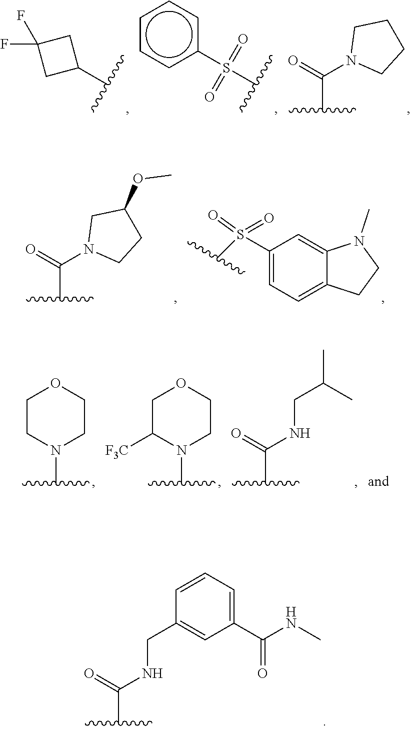

In some embodiments, for the compound or salt of Formulas (Ia), (IIa) or (IIIa), R¹⁸ is independently selected at each occurrence from:
hydrogen, C₃₋₁₀ carbocycle, and 3- to 10-membered heterocycle, wherein each C₃₋₁₀ carbocycle, and 3- to 10-membered heterocycle of R¹⁸ is optionally substituted at each occurrence with one or more substituents independently selected from:
   halogen, C₁₋₆ alkyl, C₁₋₆ haloalkyl, —OR²², —SR²², —N(R²²)₂, —C(O)R²², —C(O)OR²², —OC(O)R²², —OC(O)N(R²²)₂, —C(O)N(R²²)₂, —N(R²²)C(O)R²², —N(R²²)C(O)OR²², —N(R²²)C(O)N(R²²)₂, —N(R²²)C(S)N(R²²)₂, —N(R²²)S(O)₂(R²²), —S(O)R²², —S(O)₂R²², —S(O)₂N(R²²)₂, —NO₂, ═O, ═S, ═N(R²²), —N₃, —CN, C₃₋₆ carbocycle, and 3- to 6-membered heterocycle:
      wherein the C₃₋₆ carbocycle and 3- to 6-membered heterocycle are each optionally substituted with one or more substituents independently selected from halogen, C₁₋₄ alkyl, and C₁₋₄ haloalkyl.

In some embodiments, for the compound or salt of Formulas (Ia), (IIa) or (IIIa), R¹⁸ is independently selected at each occurrence from:
hydrogen, C₃₋₁₀ carbocycle, and 3- to 10-membered heterocycle, wherein each C₃₋₁₀ carbocycle, and 3- to 10-membered heterocycle of R¹⁸ is optionally substituted at each occurrence with one or more substituents independently selected from:
   halogen, C₁₋₆ alkyl, C₁₋₆ haloalkyl, —OR²², —N(R²²)₂, —C(O)R²², —C(O)OR²², —OC(O)R²², —OC(O)N(R²²)₂, —C(O)N(R²²)₂, —N(R²²)C(O)R²², —N(R²²)C(O)OR²², —S(O)₂R²², —S(O)₂N(R²²)₂, ═O, —CN, C₃₋₆ carbocycle, and 3- to 6-membered heterocycle;
      wherein the C₃₋₆ carbocycle and 3- to 6-membered heterocycle are each optionally substituted with one or more substituents independently selected from halogen, C₁₋₄ alkyl, and C₁₋₄ haloalkyl.

In some embodiments, for the compound or salt of Formulas (Ia), (IIa) or (IIIa), R¹⁸ is independently selected at each occurrence from: hydrogen, C₃₋₁₀ carbocycle, and 3- to 10-membered heterocycle, wherein each C₃₋₁₀ carbocycle, and 3- to 10-membered heterocycle of R¹⁸ is optionally substituted at each occurrence with one or more substituents independently selected from:
halogen, C₁₋₆ alkyl, C₁₋₆ haloalkyl, —N(R²²)₂, —C(O)R²², —C(O)N(R²²)₂, —S(O)₂R², C₃ carbocycle, and 3- to 6-membered heterocycle:
   wherein the C₃₋₆ carbocycle and 3- to 6-membered heterocycle are each optionally substituted with one or more substituents independently selected from halogen, C₁₋₄ alkyl, and C₁₋₄ haloalkyl.

In some embodiments, for the compound or salt of Formulas (Ia), (IIa) or (IIIa), R¹⁸ is independently selected at each occurrence from: hydrogen, C₃₋₁₀ carbocycle, and 3- to 10-membered heterocycle, wherein each C₃₋₁₀ carbocycle, and 3- to 10-membered heterocycle of R¹⁸ is selected from pyrrolidine, piperidine, phenyl, indoline, bicyclo[2.2.2]octane, cyclohexane, tetrahydropyran, pyridine, oxadiazole, pyrimidine, quinazoline, naphthalene, quinoline, thieno[3,2-d]pyrimidine, thieno[2,3-d]pyrimidine, benzothiazole, indane, thieno[2,3-d]pyrimidine oxide, and cyclopropyl, any of which is optionally substituted with one or more substituents independently selected from:
halogen, C₁₋₆ alkyl, C₁₋₆haloalkyl, —OR²², —SR²², —N(R²²)₂, —C(O)R²², —C(O)OR²², —OC(O)R²², —OC(O)N(R²²)₂, —C(O)N(R²²)₂, —N(R²²)C(O)R²², —N(R²)C(O)OR²², —N(R²²)C(O)N(R²²)₂,
—N(R²²)C(S)N(R²²)₂, —N(R²²)S(O)₂(R²²), —S(O)R²², —S(O)₂R²², —S(O)₂N(R²²)₂, —NO₂, ═O, ═S, ═N(R²²), —N₃, —CN, C₃₋₆ carbocycle, and 3- to 6-membered heterocycle;
   wherein the C₃₋₆ carbocycle and 3- to 6-membered heterocycle are each optionally substituted with one or more substituents independently selected from halogen, C₁₋₄ alkyl, and C₁₋₄ haloalkyl.

In some embodiments, for the compound or salt of Formulas (Ia), (IIa) or (IIIa), each C₃₋₁₀ carbocycle, and 3- to 10-membered heterocycle of R¹⁸ is selected from pyrrolidine, piperidine, phenyl, indoline, bicyclo[2.2.2]octane, cyclohexane, tetrahydropyran, pyridine, oxadiazole, pyrimidine, quinazoline, naphthalene, quinoline, thieno[3,2-d]pyrimidine, thieno[2,3-d]pyrimidine, benzothiazole, indane, thieno[2,3-d]pyrimidine oxide, and cyclopropyl, any of which is optionally substituted with one or more substituents independently selected from:
halogen, C₁₋₆ alkyl, C₁₋₆haloalkyl, —OR²², —N(R²²)₂, —C(O)R²², —C(O)OR²², —OC(O)R²², —OC(O)N(R²²)₂, —C(O)N(R²²)₂, —N(R²²)C(O)R²², —N(R²²)C(O)OR²², —S(O)₂R²², —S(O)₂N(R²)₂, ═O, —CN, C₃₋₆ carbocycle, and 3- to 6-membered heterocycle;
   wherein the C₃₋₆ carbocycle and 3- to 6-membered heterocycle are each optionally substituted with one or more substituents independently selected from halogen, C₁₋₄ alkyl, and C₁₋₄ haloalkyl.

In some embodiments, for the compound or salt of Formulas (Ia), (IIa) or (IIIa), each C₃₋₁₀ carbocycle, and 3- to 10-membered heterocycle of R¹⁸ is selected from pyrrolidine, piperidine, phenyl, indoline, bicyclo[2.2.2]octane, cyclohexane, tetrahydropyran, pyridine, oxadiazole, pyrimidine, quinazoline, naphthalene, quinoline, thieno[3,2-d]pyrimidine, thieno[2,3-d]pyrimidine, benzothiazole, indane, thieno[2,3-d]pyrimidine oxide, and cyclopropyl, any of which is optionally substituted with one or more substituents independently selected from:
halogen, C₁₋₆ alkyl, C₁₋₆ haloalkyl, —N(R²²)₂, —C(O)R²², —C(O)N(R²²)₂, —S(O)₂R²², C₃₋₆ carbocycle, and 3- to 6-membered heterocycle:
   wherein the C₃₋₆ carbocycle and 3- to 6-membered heterocycle are each optionally substituted with one or more substituents independently selected from halogen, C₁₋₄ alkyl, and C₁₋₄ haloalkyl.

In some embodiments, for the compound or salt of Formulas (Ia), (IIa) or (IIa), each C₃₋₁₀ carbocycle, and 3- to 10-membered heterocycle of R¹⁸ is selected from pyrrolidine, piperidine, phenyl, indoline, bicyclo[2.2.2]octane, cyclohexane, tetrahydropyran, pyridine, oxadiazole, pyrimidine, quinazoline, naphthalene, quinoline, thieno[3,2-d]pyrimidine, thieno[2,3-d]pyrimidine, benzothiazole, indane, thieno[2,3-d]pyrimidine oxide, and cyclopropyl, any of which is optionally substituted with one or more substituents independently selected from: halogen, methyl, trifluoromethyl, cyclopropyl, phenyl, —NH₂, ═O,

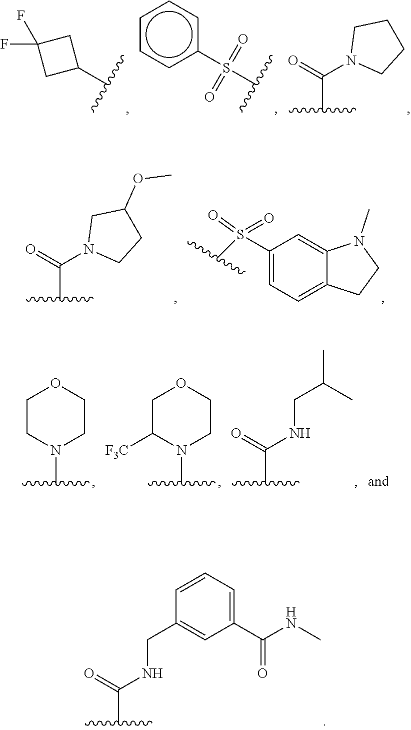

In some embodiments, for the compound or salt of Formulas (Ia), (IIa) or (IIa), each C₃₋₁₀ carbocycle and 3- to 10-membered heterocycle of R¹⁸ is independently selected at each occurrence from monocyclic C₃₋₆ carbocycle and 3- to 7-membered monocyclic heterocycle any of which is optionally substituted at each occurrence with one or more substituents independently selected from:
hydrogen, C₃₋₁₀ carbocycle, and 3- to 10-membered heterocycle, wherein each C₃₋₁₀ carbocycle, and 3- to 10-membered heterocycle of R¹⁸ is optionally substituted at each occurrence with one or more substituents independently selected from:
   halogen, C₁₋₆ alkyl, C₁₋₆ haloalkyl, —OR²², —N(R²²)₂, —C(O)R²², —C(O)OR², —OC(O)R²², —OC(O)N(R²)₂, —C(O)N(R²)₂, —N(R²)C(O)R²², —N(R²²)C(O)OR²², —S(O)₂R²², —S(O)₂N(R²²)₂, ═O, —CN, C₃₋₆ carbocycle, and 3- to 6-membered heterocycle;
      wherein the C₃₋₆ carbocycle and 3- to 6-membered heterocycle are each optionally substituted with one or more substituents independently selected from halogen, C₁₋₄ alkyl, and C₁₋₄ haloalkyl.

In some embodiments, for the compound or salt of Formulas (Ia), (IIa) or (IIIa), each C₃₋₁₀ carbocycle and 3- to 10-membered heterocycle of R¹¹ is independently selected at each occurrence from monocyclic C₃₋₆ carbocycle and 3- to 7-membered monocyclic heterocycle any of which is optionally substituted at each occurrence with one or more substituents independently selected from:
halogen, C₁₋₆ alkyl, C₁₋₆ haloalkyl, —N(R²²)₂, —C(O)R²², —C(O)N(R²²)₂, —S(O)₂R²², C₃ carbocycle, and 3- to 6-membered heterocycle;
   wherein the C₃₋₆ carbocycle and 3- to 6-membered heterocycle are each optionally substituted with one or more substituents independently selected from halogen, C₁₋₄ alkyl, and C₁₋₄ haloalkyl.

In some embodiments, for the compound or salt of Formulas (Ia), (IIa) or (IIIa), each C₃₋₁₀ carbocycle and 3- to 10-membered heterocycle of R¹⁸ is independently selected at each occurrence from monocyclic C₃₋₆ carbocycle and 3- to 7-membered monocyclic heterocycle any of which is optionally substituted at each occurrence with one or more substituents independently selected from: halogen, methyl, trifluoromethyl, cyclopropyl, phenyl, —NH₂, ═O,

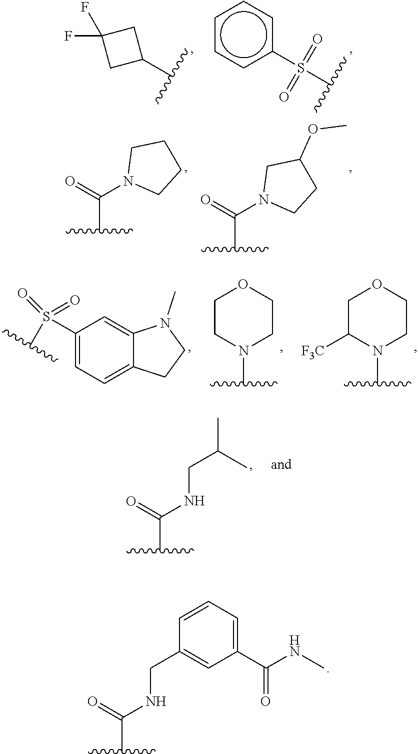

In some embodiments, for the compound or salt of Formulas (Ia), (IIa) or (IIIa), each C₃₋₁₀ carbocycle and 3- to 10-membered heterocycle of R¹⁸ is independently selected at each occurrence from pyrrolidine, piperidine, phenyl, cyclohexane, tetrahydropyran, pyridine, oxadiazole, pyrimidine, and cyclopropyl, any of which is optionally substituted at each occurrence with one or more substituents independently selected from:
hydrogen, C₃₋₁₀ carbocycle, and 3- to 10-membered heterocycle, wherein each C₃₋₁₀ carbocycle, and 3- to 10-membered heterocycle of R¹⁸ is optionally substituted at each occurrence with one or more substituents independently selected from:
   halogen, C₁₋₆ alkyl, C₁₋₆ haloalkyl, —OR²², —N(R²²)₂, —C(O)R²², —C(O)OR²², —OC(O)R²², —OC(O)N(R²²), —C(O)N(R²²)₂, —N(R¹²)C(O)R²², —N(R²²)C(O)OR²², —S(O)₂R²², —S(O)₂N(R²²)₂, ═O, —CN, C₃, carbocycle, and 3- to 6-membered heterocycle:
      wherein the C₃₋₆ carbocycle and 3- to 6-membered heterocycle are each optionally substituted with one or more substituents independently selected from halogen, C₁₋₄ alkyl, and C₁₋₄ haloalkyl.

In some embodiments, for the compound or salt of Formulas (Ia), (IIa) or (IIIa), each C₃₋₁₀ carbocycle and 3- to 10-membered heterocycle of R¹⁸ is independently selected at each occurrence from pyrrolidine, piperidine, phenyl, cyclohexane, tetrahydropyran, pyridine, oxadiazole, pyrimidine, and cyclopropyl, any of which is optionally substituted at each occurrence with one or more substituents independently selected from:
halogen, C₁₋₆ alkyl, C₁₋₆ haloalkyl, —N(R²²)₂, —C(O)R²², —C(O)N(R²²)₂, —S(O)₂R¹¹, C₃₋₆ carbocycle, and 3- to 6-membered heterocycle;
   wherein the C₃₋₆ carbocycle and 3- to 6-membered heterocycle are each optionally substituted with one or more substituents independently selected from halogen, C₁₋₄ alkyl, and C₁₋₄ haloalkyl.

In some embodiments, for the compound or salt of Formulas (Ia), (IIa) or (IIIa), each C₃₋₁₀ carbocycle and 3- to 10-membered heterocycle of R¹⁸ is independently selected at each occurrence from pyrrolidine, piperidine, phenyl, cyclohexane, tetrahydropyran, pyridine, oxadiazole, pyrimidine, and cyclopropyl, any of which is optionally substituted at each occurrence with one or more substituents independently selected from: halogen, methyl, trifluoromethyl, cyclopropyl, phenyl, —NH₂, ═O,

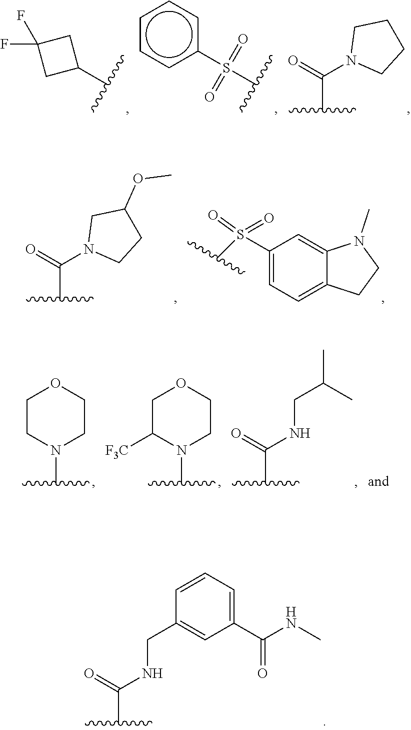

In some embodiments, for the compound or salt of Formulas (Ia), (IIa) or (IIIa), each C₃₋₁₀ carbocycle and 3- to 10-membered heterocycle of R¹⁸ is independently selected at each occurrence from bicyclic C₆₋₁₀ carbocycle and bicyclic 6- to 10-membered bicyclic heterocycle, any of which is optionally substituted at each occurrence with one or more substituents independently selected from:
hydrogen, C₃₋₁₀ carbocycle, and 3- to 10-membered heterocycle, wherein each C₃₋₁₀ carbocycle, and 3- to 10-membered heterocycle of R¹⁸ is optionally substituted at each occurrence with one or more substituents independently selected from:
   halogen, C₁₋₆ alkyl, C₁₋₆ haloalkyl, —OR²², —N(R²²)₂, —C(O)R²², —C(O)OR², —OC(O)R²², —OC(O)N(R²)₂, —C(O)N(R²²)₂, —N(R²)C(O)R²², —N(R²²)C(O)OR²², —S(O)₂R²², —S(O)₂N(R²²)₂, ═O, —CN, C₃₋₆ carbocycle, and 3- to 6-membered heterocycle;
      wherein the C₃₋₆ carbocycle and 3- to 6-membered heterocycle are each optionally substituted with one or more substituents independently selected from halogen, C₁₋₄ alkyl, and C₁₋₄ haloalkyl.

In some embodiments, for the compound or salt of Formulas (Ia), (IIa) or (IIIa), each C₃₋₁₀ carbocycle and 3- to 10-membered heterocycle of R¹⁸ is independently selected at each occurrence from bicyclic C₆₋₁₀ carbocycle and bicyclic 6- to 10-membered bicyclic heterocycle, any of which is optionally substituted at each occurrence with one or more substituents independently selected from:
halogen, C₁₋₆ alkyl, C₁₋₆ haloalkyl, —N(R²)₂, —C(O)R²², —C(O)N(R²²)₂, —S(O)₂R²², C₃₋₆ carbocycle, and 3- to 6-membered heterocycle;
   wherein the C₃₋₆ carbocycle and 3- to 6-membered heterocycle are each optionally substituted with one or more substituents independently selected from halogen, C₁₋₄ alkyl, and C₁₋₄ haloalkyl.

In some embodiments, for the compound or salt of Formulas (Ia), (IIa) or (IIIa), each C₃₋₁₀ carbocycle and 3- to 10-membered heterocycle of R¹⁸ is independently selected at each occurrence from bicyclic C₆₋₁₀ carbocycle and bicyclic 6- to 10-membered bicyclic heterocycle, any of which is optionally substituted at each occurrence with one or more substituents independently selected from: halogen, methyl, trifluoromethyl, cyclopropyl, phenyl, —NH₂, ═O,

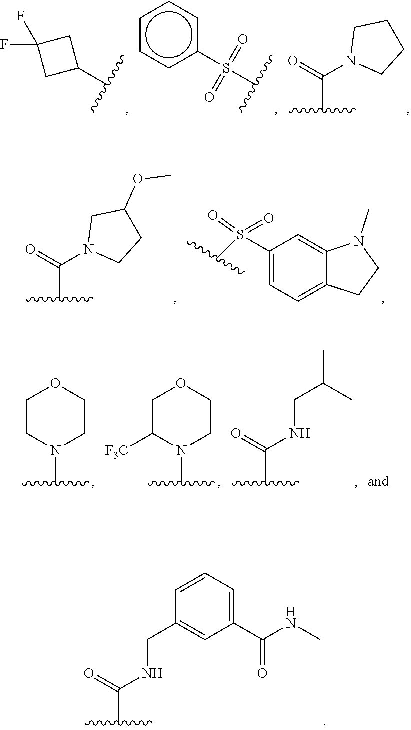

In some embodiments, for the compound or salt of Formulas (Ia), (IIa) or (IIIa), each C₃₋₁₀ carbocycle and 3- to 10-membered heterocycle of R¹⁸ is independently selected at each occurrence from indoline, bicyclo[2.2.2]octane, quinazoline, naphthalene, quinoline, thieno[3,2-d]pyrimidine, thieno[2,3-d]pyrimidine, benzothiazole, indane, and thieno[2,3-d]pyrimidine oxide, any of which is optionally substituted with one or more substituents independently selected from: hydrogen, C₃₋₁₀ carbocycle, and 3- to 10-membered heterocycle, wherein each C₃₋₁₀ carbocycle, and 3- to 10-membered heterocycle of R¹⁸ is optionally substituted at each occurrence with one or more substituents independently selected from:
halogen, C₁₋₆ alkyl, C₁₋₆haloalkyl, —OR²², —N(R²²)₂, —C(O)R²², —C(O)OR²², —OC(O)R²², —OC(O)N(R²²)₂, —C(O)N(R²)₂, —N(R²²)C(O)R²², —N(R²²)C(O)OR²², —S(O)₂R²², —S(O)₂N(R²²)₂, ═O, —CN, C₃₋₆ carbocycle, and 3- to 6-membered heterocycle;
   wherein the C₃₋₆ carbocycle and 3- to 6-membered heterocycle are each optionally substituted with one or more substituents independently selected from halogen, C₁₋₄ alkyl, and C₁₋₄ haloalkyl.

In some embodiments, for the compound or salt of Formulas (Ia), (IIa) or (IIIa), each C₃₋₁₀ carbocycle and 3- to 10-membered heterocycle of R¹⁸ is independently selected at each occurrence from indoline, bicyclo[2.2.2]octane, quinazoline, naphthalene, quinoline, thieno[3,2-d]pyrimidine, thieno[2,3-d]pyrimidine, benzothiazole, indane, and thieno[2,3-d]pyrimidine oxide, any of which is optionally substituted with one or more substituents independently selected from:
halogen, C₁₋₆ alkyl, C₁₋₆ haloalkyl, —N(R²²)₂, —C(O)R²², —C(O)N(R²²)₂, —S(O)₂R²², C₃ carbocycle, and 3- to 6-membered heterocycle:
   wherein the C₃₋₆ carbocycle and 3- to 6-membered heterocycle are each optionally substituted with one or more substituents independently selected from halogen, C₁₋₄ alkyl, and C₁₋₄ haloalkyl.

In some embodiments, for the compound or salt of Formulas (Ia), (IIa) or (IIIa), each C₃₋₁₀ carbocycle and 3- to 10-membered heterocycle of R¹⁸ is independently selected at each occurrence from indoline, bicyclo[2.2.2]octane, quinazoline, naphthalene, quinoline, thieno[3,2-d]pyrimidine, thieno[2,3-d]pyrimidine, benzothiazole, indane, and thieno[2,3-d]pyrimidine oxide, any of which is optionally substituted with one or more substituents independently selected from: halogen, methyl, trifluoromethyl, cyclopropyl, phenyl, —NH₂, ═O,

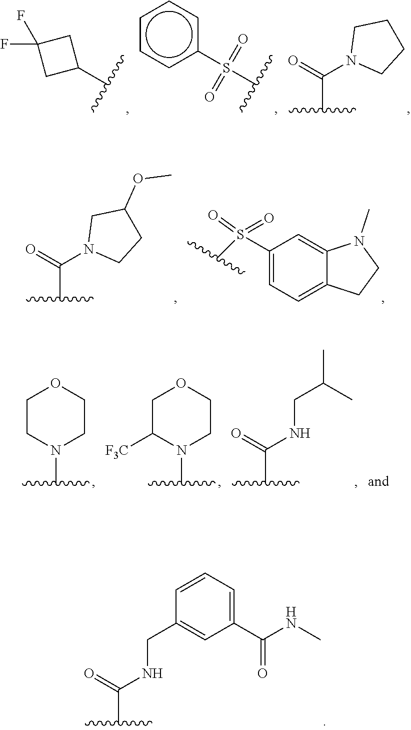

In some embodiments, for the compound or salt of Formulas (Ia), (IIa) or (IIIa), B is selected from

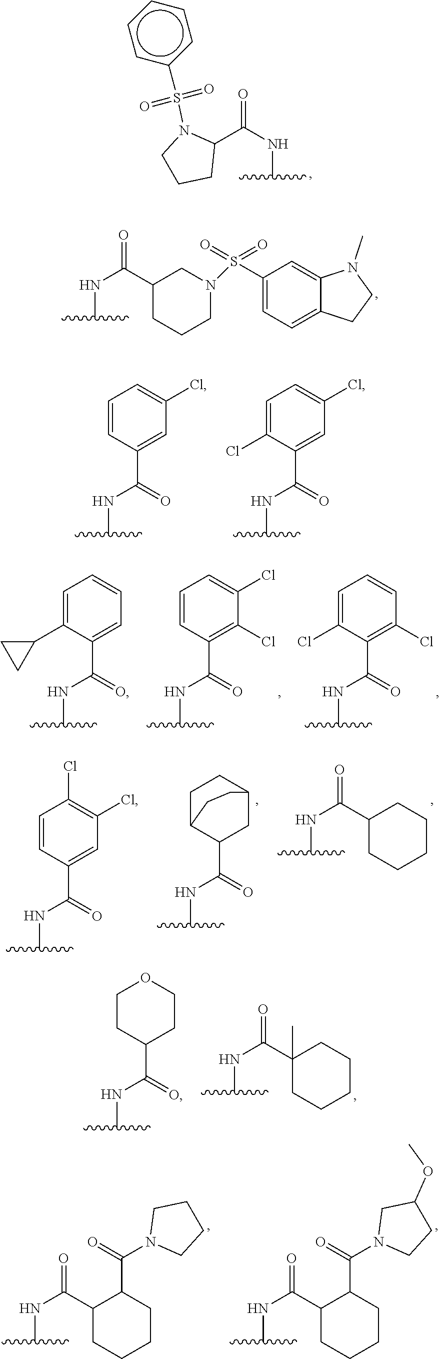

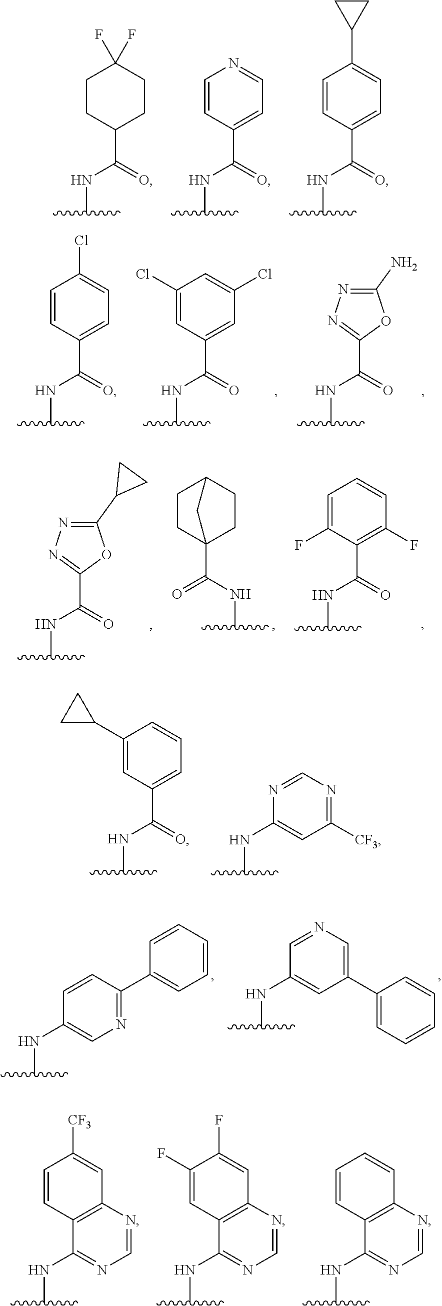

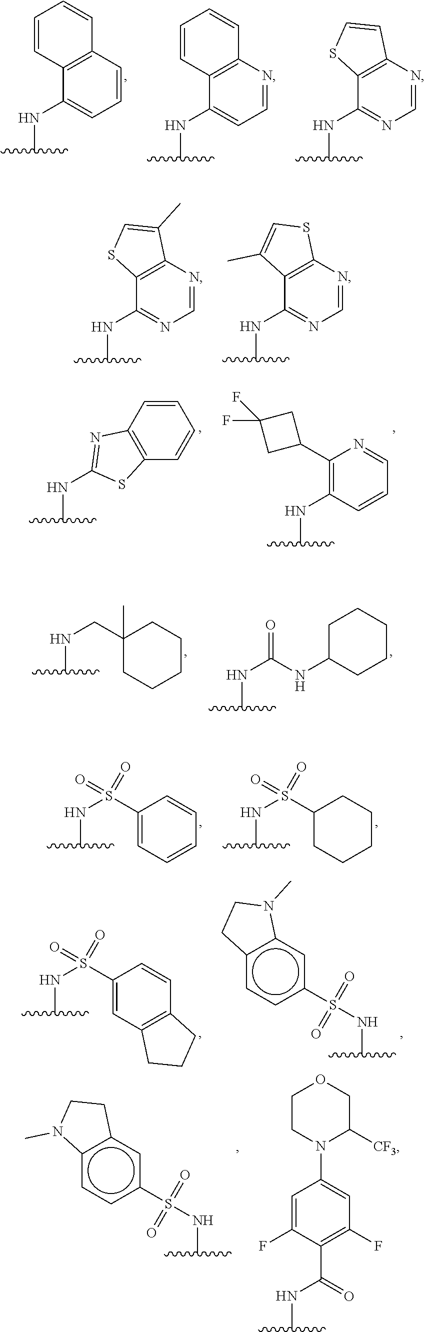

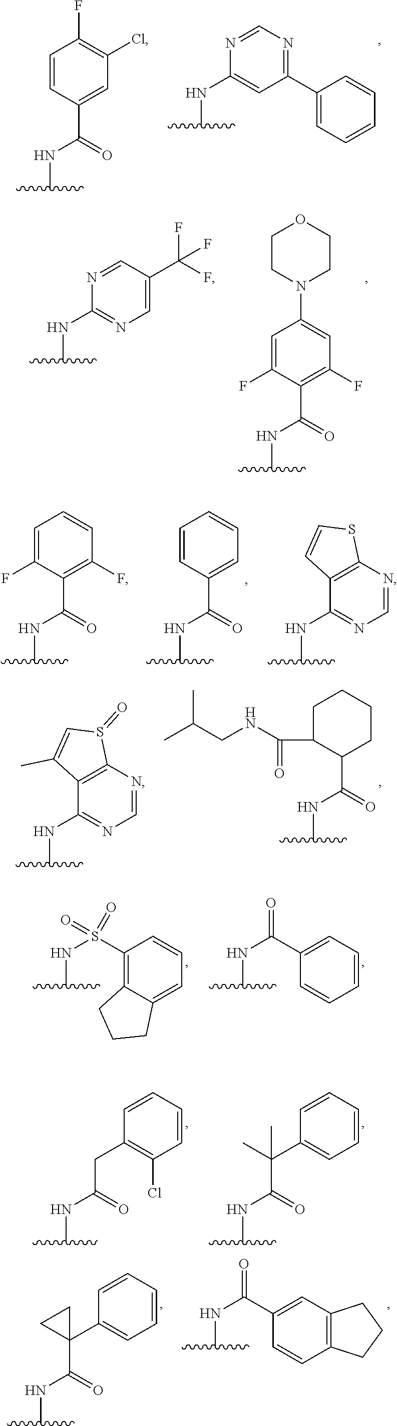

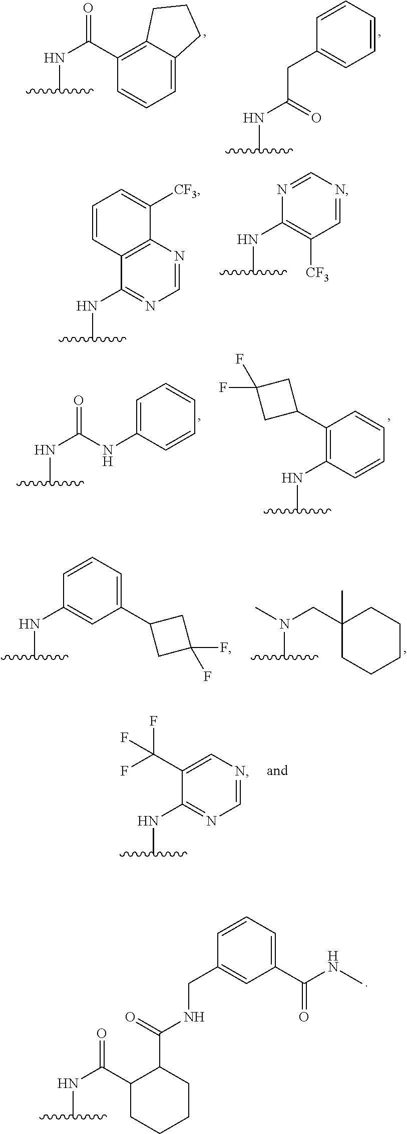

In some embodiments, for the compound or salt of Formulas (Ia), (IIa) or (IIIa), B is selected from

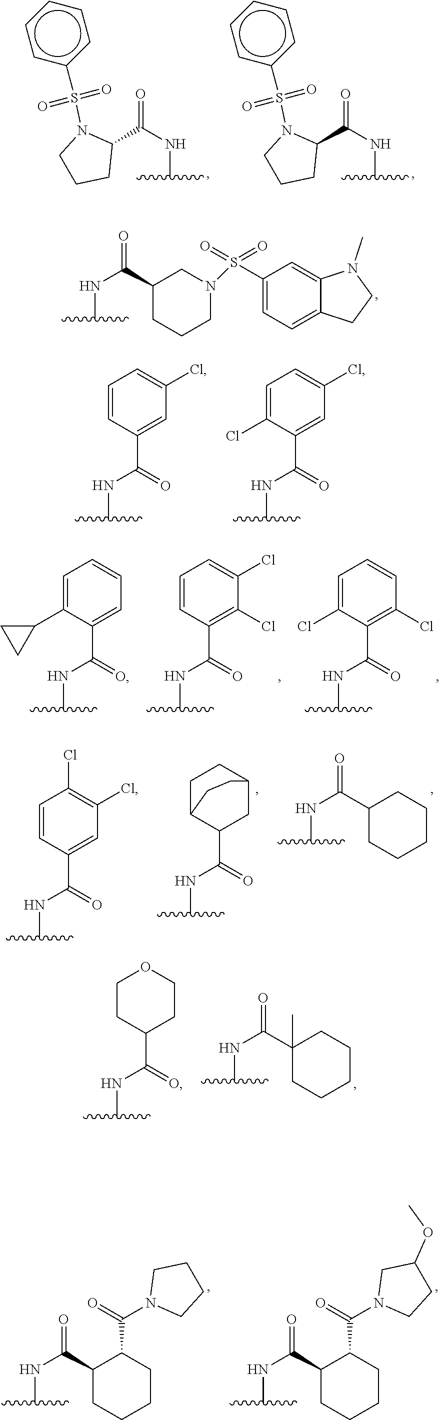

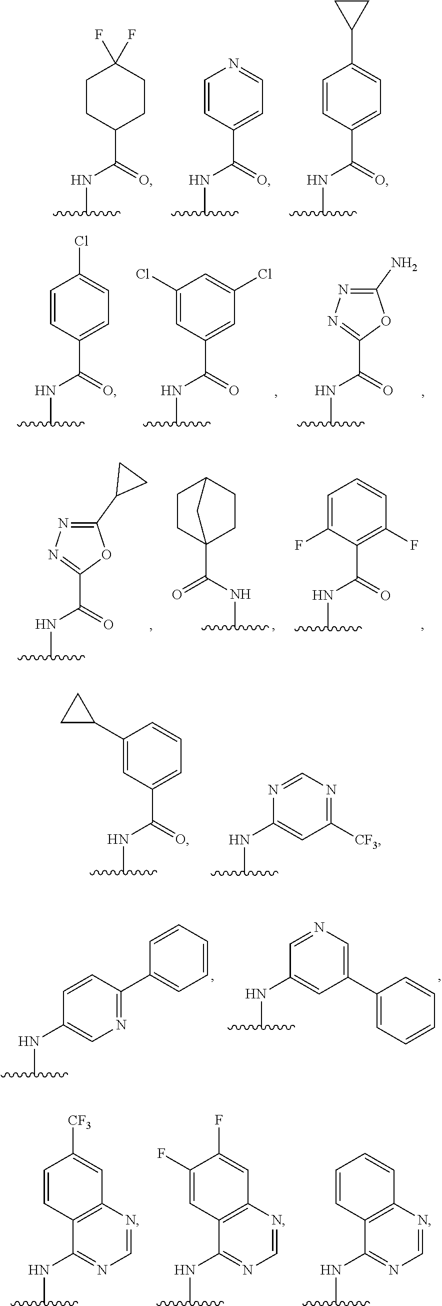

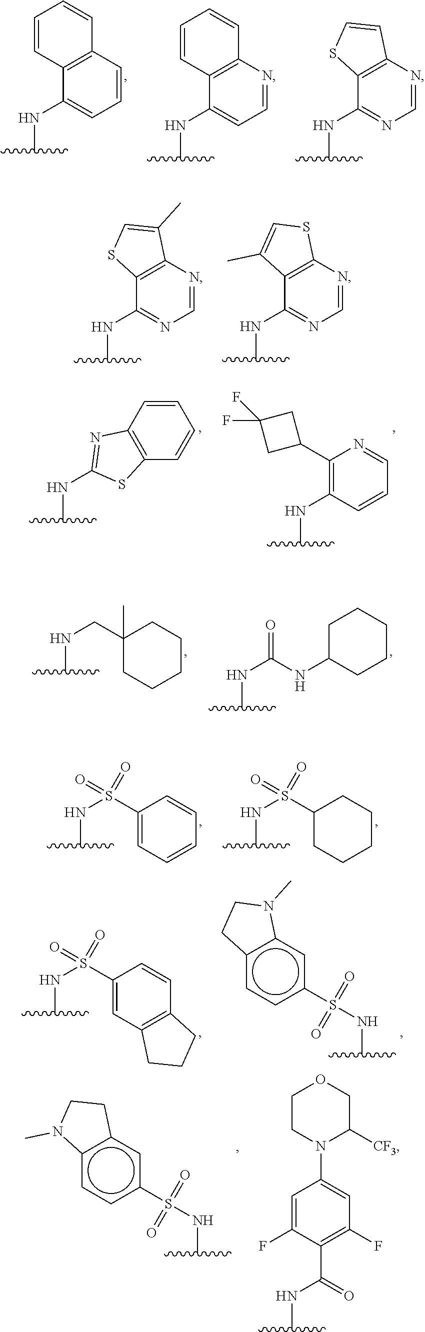

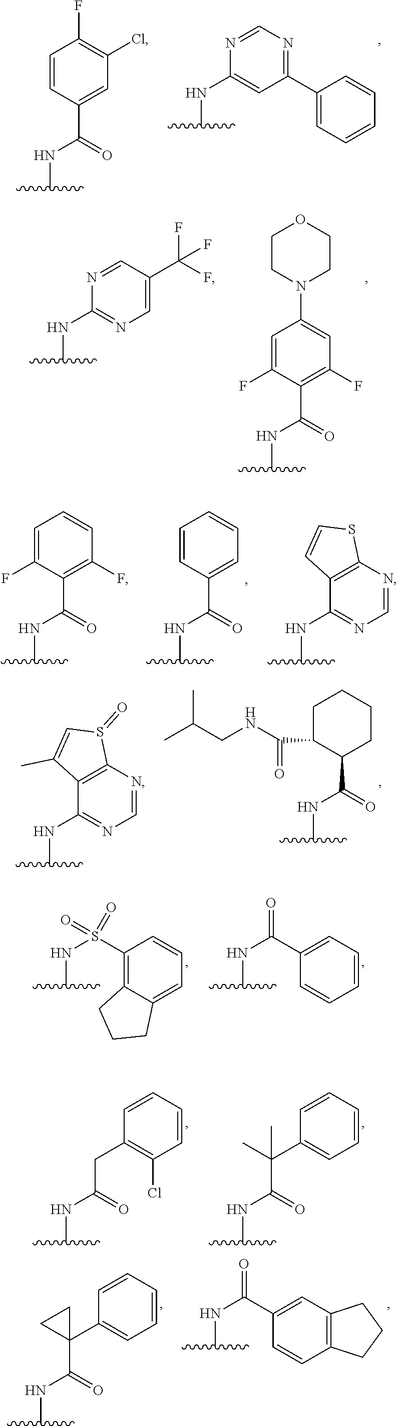

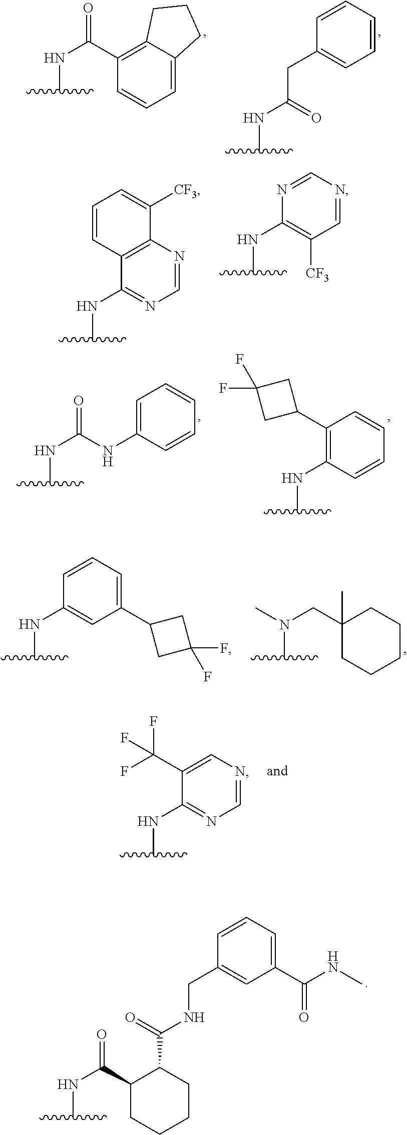

In some embodiments, the compound or salt of Formula (I), (II) or (111) is selected from a compound in Table 1, below:

TABLE 1

| # | Structure |
|---|---|
| 201 | 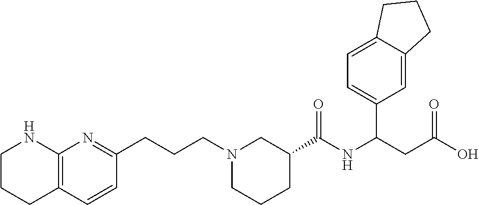 [SMILES: O=C(O)CC(NC(=O)[C@@H]1CCCN(CCCc2ccc3c(n2)NCCC3)C1)c1ccc2c(c1)CCC2] |
| 201-A | 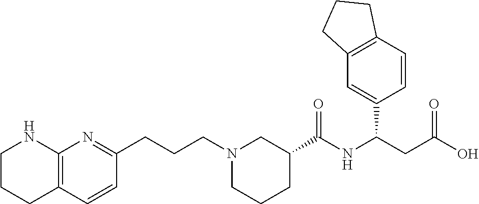 [SMILES: O=C(O)C[C@H](NC(=O)[C@@H]1CCCN(CCCc2ccc3c(n2)NCCC3)C1)c1ccc2c(c1)CCC2] |
| 201-B | 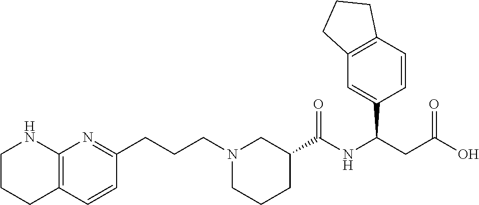 [SMILES: O=C(O)C[C@@H](NC(=O)[C@@H]1CCCN(CCCc2ccc3c(n2)NCCC3)C1)c1ccc2c(c1)CCC2] |
| 202 | 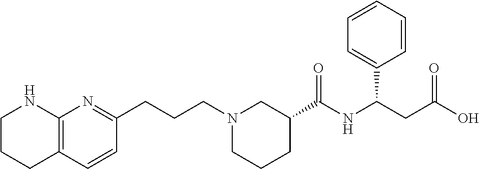 [SMILES: O=C(O)C[C@H](NC(=O)[C@@H]1CCCN(CCCc2ccc3c(n2)NCCC3)C1)c1ccccc1] |
| 203 | 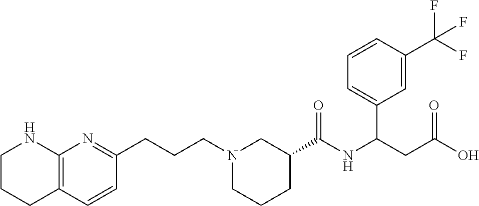 [SMILES: O=C(O)CC(NC(=O)[C@@H]1CCCN(CCCc2ccc3c(n2)NCCC3)C1)c1cccc(C(F)(F)F)c1] |
| 203-A | 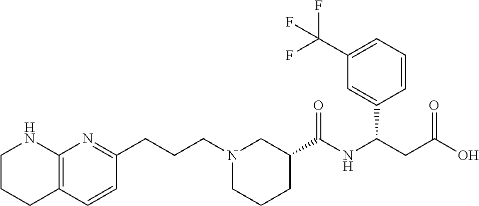 [SMILES: O=C(O)C[C@H](NC(=O)[C@@H]1CCCN(CCCc2ccc3c(n2)NCCC3)C1)c1cccc(C(F)(F)F)c1] |
| 203-B | 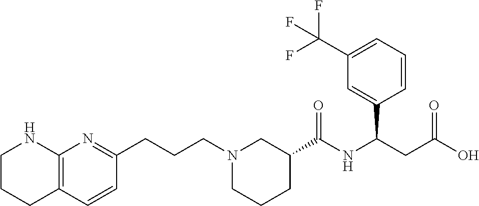 [SMILES: O=C(O)C[C@@H](NC(=O)[C@@H]1CCCN(CCCc2ccc3c(n2)NCCC3)C1)c1cccc(C(F)(F)F)c1] |
| 204 | 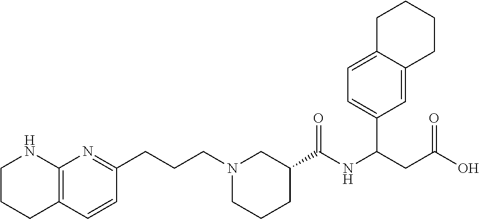 [SMILES: O=C(O)CC(NC(=O)[C@@H]1CCCN(CCCc2ccc3c(n2)NCCC3)C1)c1ccc2c(c1)CCCC2] |
| 204-A | 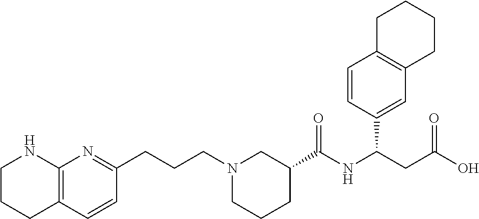 [SMILES: O=C(O)C[C@H](NC(=O)[C@@H]1CCCN(CCCc2ccc3c(n2)NCCC3)C1)c1ccc2c(c1)CCCC2] |
| 204-B | 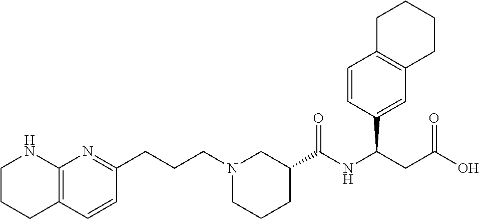 [SMILES: O=C(O)C[C@@H](NC(=O)[C@@H]1CCCN(CCCc2ccc3c(n2)NCCC3)C1)c1ccc2c(c1)CCCC2] |
| 205 | 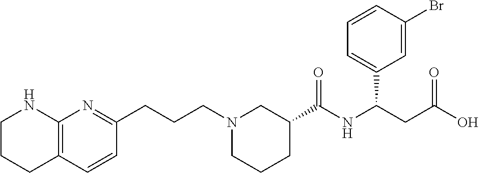 [SMILES: O=C(O)C[C@H](NC(=O)[C@@H]1CCCN(CCCc2ccc3c(n2)NCCC3)C1)c1cccc(Br)c1] |
| 206 | 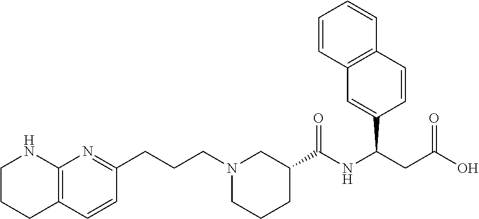 [SMILES: O=C(O)C[C@@H](NC(=O)[C@@H]1CCCN(CCCc2ccc3c(n2)NCCC3)C1)c1ccc2ccccc2c1] |
| 207 | 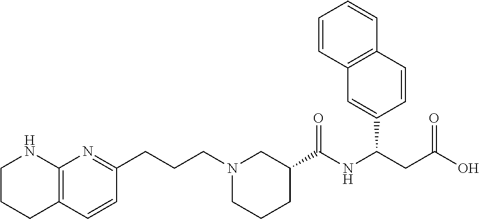 [SMILES: O=C(O)C[C@H](NC(=O)[C@@H]1CCCN(CCCc2ccc3c(n2)NCCC3)C1)c1ccc2ccccc2c1] |
| 208 | 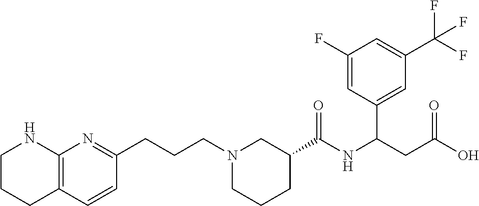 [SMILES: O=C(O)CC(NC(=O)[C@@H]1CCCN(CCCc2ccc3c(n2)NCCC3)C1)c1cc(F)cc(C(F)(F)F)c1] |
| 208-A | 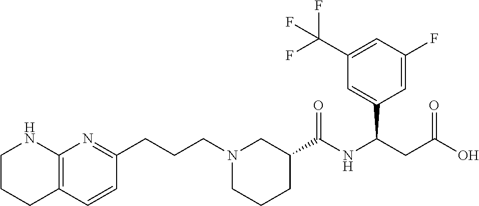 [SMILES: O=C(O)C[C@@H](NC(=O)[C@@H]1CCCN(CCCc2ccc3c(n2)NCCC3)C1)c1cc(F)cc(C(F)(F)F)c1] |
| 208-B | 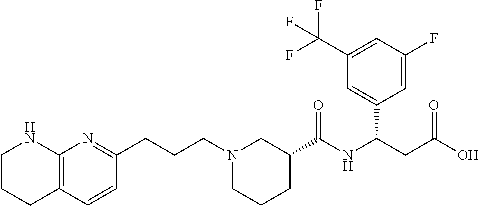 [SMILES: O=C(O)C[C@H](NC(=O)[C@@H]1CCCN(CCCc2ccc3c(n2)NCCC3)C1)c1cc(F)cc(C(F)(F)F)c1] |
| 209 | 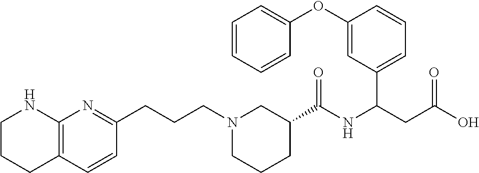 [SMILES: O=C(O)CC(NC(=O)[C@@H]1CCCN(CCCc2ccc3c(n2)NCCC3)C1)c1cccc(Oc2ccccc2)c1] |
| 209-A | 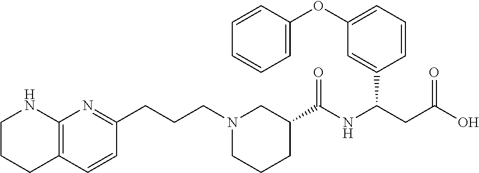 [SMILES: O=C(O)C[C@H](NC(=O)[C@@H]1CCCN(CCCc2ccc3c(n2)NCCC3)C1)c1cccc(Oc2ccccc2)c1] |
| 209-B | 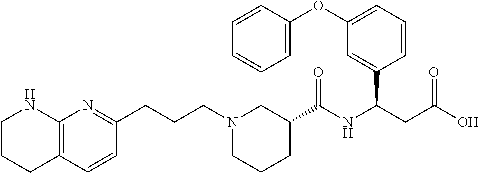 [SMILES: O=C(O)C[C@@H](NC(=O)[C@@H]1CCCN(CCCc2ccc3c(n2)NCCC3)C1)c1cccc(Oc2ccccc2)c1] |
| 210 | 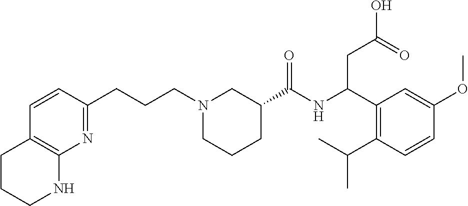 [SMILES: COc1ccc(C(C)C)c(C(CC(=O)O)NC(=O)[C@@H]2CCCN(CCCc3ccc4c(n3)NCCC4)C2)c1] |
| 211 | 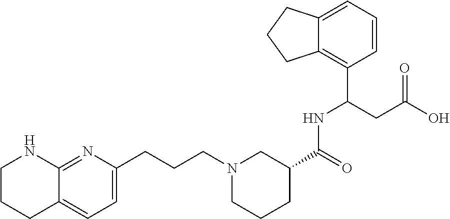 [SMILES: O=C(O)CC(NC(=O)[C@@H]1CCCN(CCCc2ccc3c(n2)NCCC3)C1)c1cccc2c1CCC2] |
| 211-A | 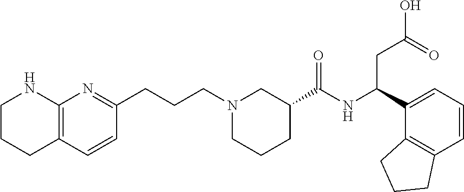 [SMILES: O=C(O)C[C@H](NC(=O)[C@@H]1CCCN(CCCc2ccc3c(n2)NCCC3)C1)c1cccc2c1CCC2] |
| 211-B | 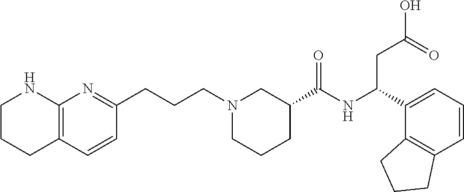 [SMILES: O=C(O)C[C@@H](NC(=O)[C@@H]1CCCN(CCCc2ccc3c(n2)NCCC3)C1)c1cccc2c1CCC2] |
| 212 | 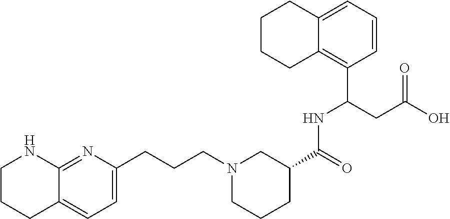 [SMILES: O=C(O)CC(NC(=O)[C@@H]1CCCN(CCCc2ccc3c(n2)NCCC3)C1)c1cccc2c1CCCC2] |
| 212-A | 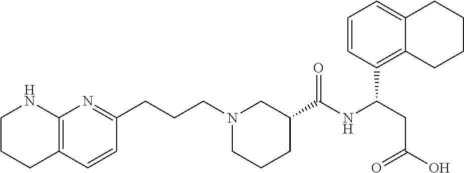 [SMILES: O=C(O)C[C@H](NC(=O)[C@@H]1CCCN(CCCc2ccc3c(n2)NCCC3)C1)c1cccc2c1CCCC2] |
| 212-B | 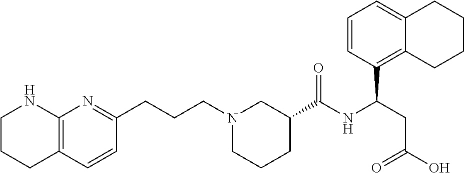 [SMILES: O=C(O)C[C@@H](NC(=O)[C@@H]1CCCN(CCCc2ccc3c(n2)NCCC3)C1)c1cccc2c1CCCC2] |
| 213 | 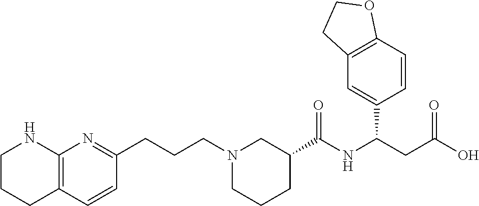 [SMILES: O=C(O)C[C@H](NC(=O)[C@@H]1CCCN(CCCc2ccc3c(n2)NCCC3)C1)c1ccc2c(c1)CCO2] |
| 214 | 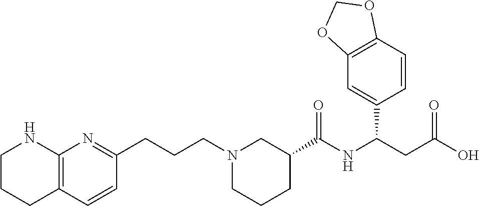 [SMILES: O=C(O)C[C@H](NC(=O)[C@@H]1CCCN(CCCc2ccc3c(n2)NCCC3)C1)c1ccc2c(c1)OCO2] |
| 215 | 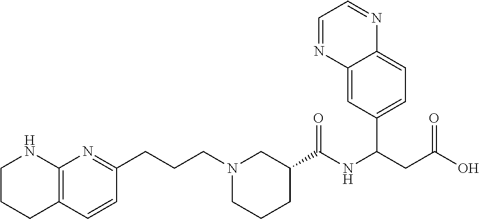 [SMILES: O=C(O)CC(NC(=O)[C@@H]1CCCN(CCCc2ccc3c(n2)NCCC3)C1)c1ccc2nccnc2c1] |
| 215-A | 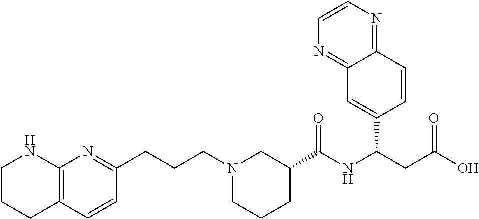 [SMILES: O=C(O)C[C@H](NC(=O)[C@@H]1CCCN(CCCc2ccc3c(n2)NCCC3)C1)c1ccc2nccnc2c1] |
| 215-B | 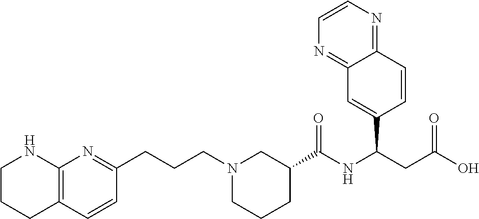 [SMILES: O=C(O)C[C@@H](NC(=O)[C@@H]1CCCN(CCCc2ccc3c(n2)NCCC3)C1)c1ccc2nccnc2c1] |
| 216 | 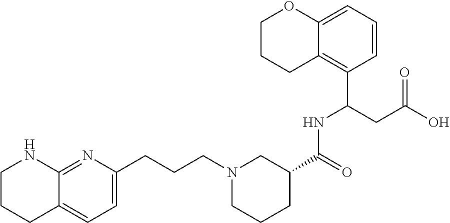 [SMILES: O=C(O)CC(NC(=O)[C@@H]1CCCN(CCCc2ccc3c(n2)NCCC3)C1)c1cccc2c1CCCO2] |
| 216-A | 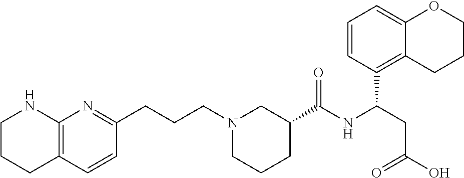 [SMILES: O=C(O)C[C@H](NC(=O)[C@@H]1CCCN(CCCc2ccc3c(n2)NCCC3)C1)c1cccc2c1CCCO2] |
| 216-B | 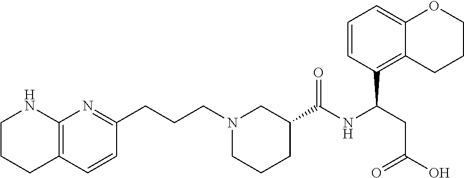 [SMILES: O=C(O)C[C@@H](NC(=O)[C@@H]1CCCN(CCCc2ccc3c(n2)NCCC3)C1)c1cccc2c1CCCO2] |
| 217 | 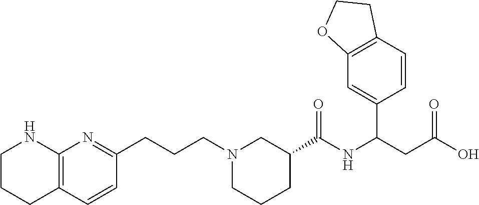 [SMILES: O=C(O)CC(NC(=O)[C@@H]1CCCN(CCCc2ccc3c(n2)NCCC3)C1)c1ccc2c(c1)OCC2] |
| 218 | 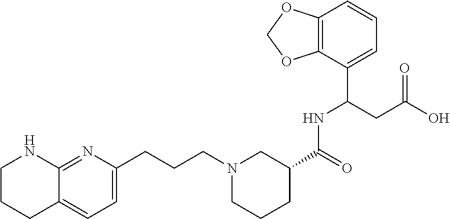 [SMILES: O=C(O)CC(NC(=O)[C@@H]1CCCN(CCCc2ccc3c(n2)NCCC3)C1)c1cccc2c1OCO2] |
| 219 | 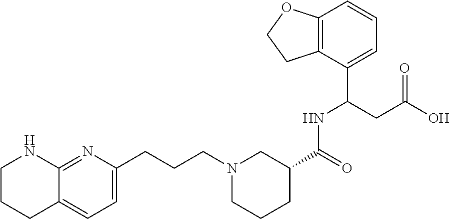 [SMILES: O=C(O)CC(NC(=O)[C@@H]1CCCN(CCCc2ccc3c(n2)NCCC3)C1)c1cccc2c1CCO2] |
| 220 | 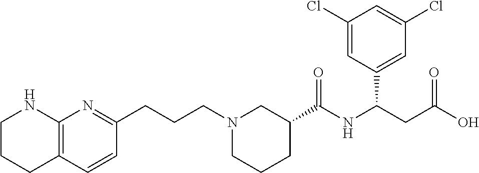 [SMILES: O=C(O)C[C@H](NC(=O)[C@@H]1CCCN(CCCc2ccc3c(n2)NCCC3)C1)c1cc(Cl)cc(Cl)c1] |
| 221 | 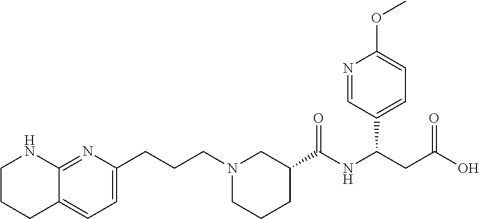 [SMILES: COc1ccc([C@H](CC(=O)O)NC(=O)[C@@H]2CCCN(CCCc3ccc4c(n3)NCCC4)C2)cn1] |
| 222 | 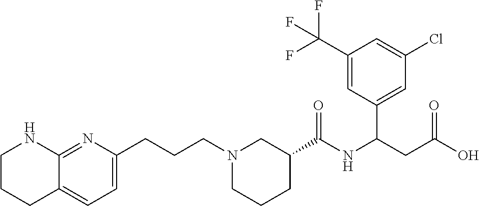 [SMILES: O=C(O)CC(NC(=O)[C@@H]1CCCN(CCCc2ccc3c(n2)NCCC3)C1)c1cc(Cl)cc(C(F)(F)F)c1] |
| 223 | 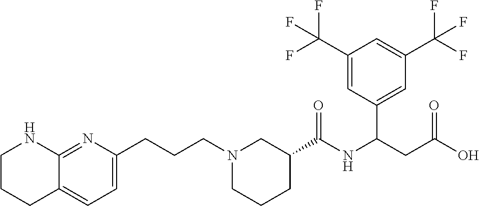 [SMILES: O=C(O)CC(NC(=O)[C@@H]1CCCN(CCCc2ccc3c(n2)NCCC3)C1)c1cc(C(F)(F)F)cc(C(F)(F)F)c1] |
| 224 | 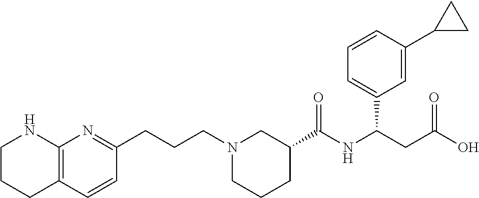 [SMILES: O=C(O)C[C@H](NC(=O)[C@@H]1CCCN(CCCc2ccc3c(n2)NCCC3)C1)c1cccc(C2CC2)c1] |
| 225 | 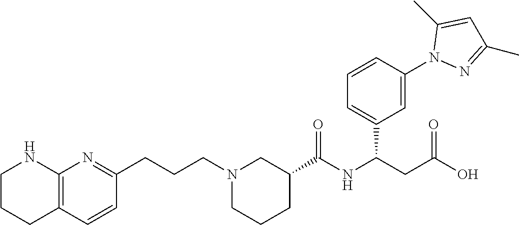 [SMILES: Cc1cc(C)n(-c2cccc([C@H](CC(=O)O)NC(=O)[C@@H]3CCCN(CCCc4ccc5c(n4)NCCC5)C3)c2)n1] |
| 226 | 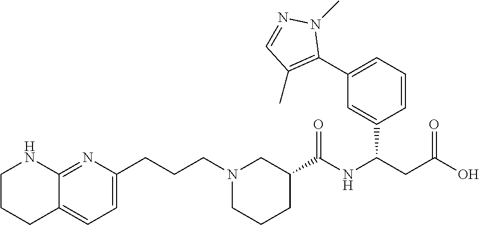 [SMILES: Cc1cnn(C)c1-c1cccc([C@H](CC(=O)O)NC(=O)[C@@H]2CCCN(CCCc3ccc4c(n3)NCCC4)C2)c1] |
| 227 | 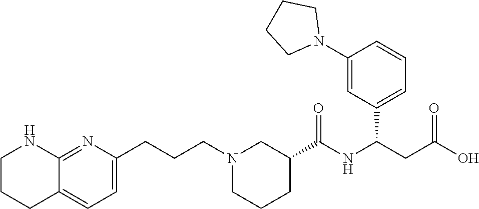 [SMILES: O=C(O)C[C@H](NC(=O)[C@@H]1CCCN(CCCc2ccc3c(n2)NCCC3)C1)c1cccc(N2CCCC2)c1] |
| 228 | 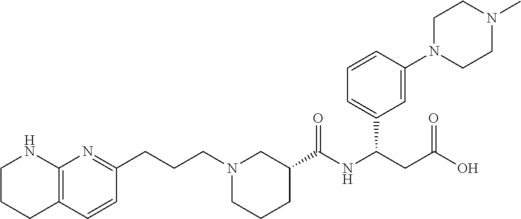 [SMILES: CN1CCN(c2cccc([C@H](CC(=O)O)NC(=O)[C@@H]3CCCN(CCCc4ccc5c(n4)NCCC5)C3)c2)CC1] |
| 229 | 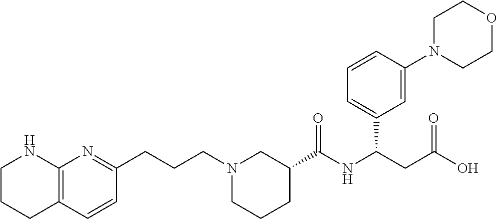 [SMILES: O=C(O)C[C@H](NC(=O)[C@@H]1CCCN(CCCc2ccc3c(n2)NCCC3)C1)c1cccc(N2CCOCC2)c1] |
| 230 | 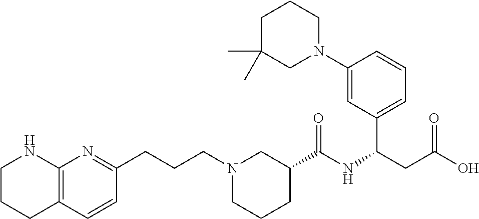 [SMILES: CC1(C)CCCN(c2cccc([C@H](CC(=O)O)NC(=O)[C@@H]3CCCN(CCCc4ccc5c(n4)NCCC5)C3)c2)C1] |
| 231 | 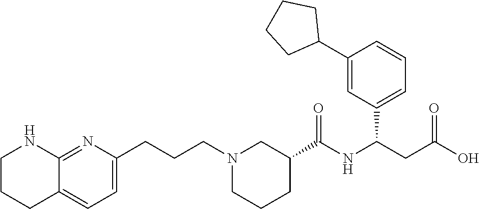 [SMILES: O=C(O)C[C@H](NC(=O)[C@@H]1CCCN(CCCc2ccc3c(n2)NCCC3)C1)c1cccc(C2CCCC2)c1] |
| 232 | 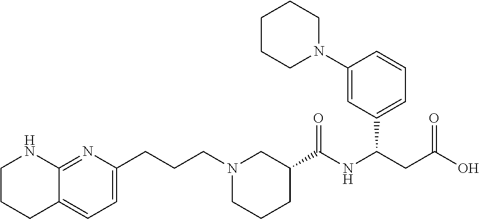 [SMILES: O=C(O)C[C@H](NC(=O)[C@@H]1CCCN(CCCc2ccc3c(n2)NCCC3)C1)c1cccc(N2CCCCC2)c1] |
| 233 | 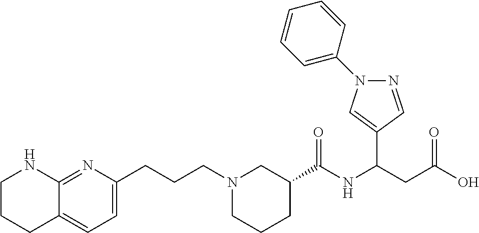 [SMILES: O=C(O)CC(NC(=O)[C@@H]1CCCN(CCCc2ccc3c(n2)NCCC3)C1)c1cnn(-c2ccccc2)c1] |
| 234 | 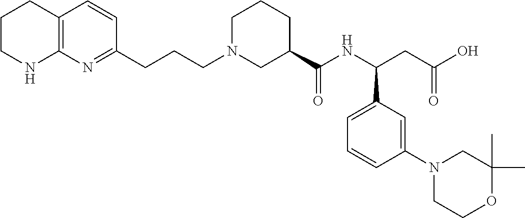 [SMILES: CC1(C)CN(c2cccc([C@H](CC(=O)O)NC(=O)[C@@H]3CCCN(CCCc4ccc5c(n4)NCCC5)C3)c2)CCO1] |
| 235 | 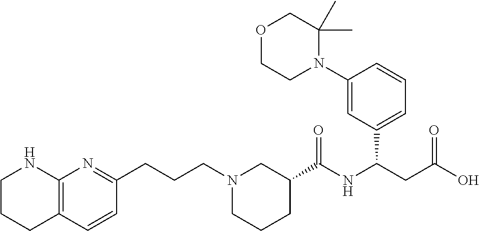 [SMILES: CC1(C)COCCN1c1cccc([C@H](CC(=O)O)NC(=O)[C@@H]2CCCN(CCCc3ccc4c(n3)NCCC4)C2)c1] |
| 236 | 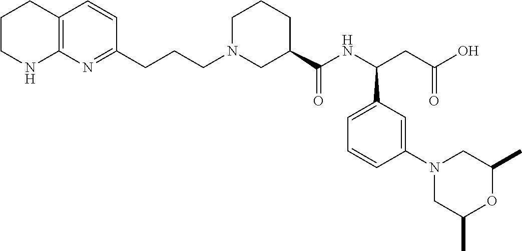 [SMILES: C[C@@H]1CN(c2cccc([C@H](CC(=O)O)NC(=O)[C@@H]3CCCN(CCCc4ccc5c(n4)NCCC5)C3)c2)C[C@H](C)O1] |
| 237 | 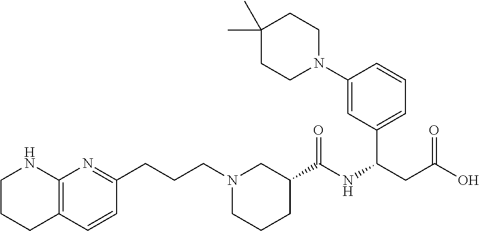 [SMILES: CC1(C)CCN(c2cccc([C@H](CC(=O)O)NC(=O)[C@@H]3CCCN(CCCc4ccc5c(n4)NCCC5)C3)c2)CC1] |
| 238 | 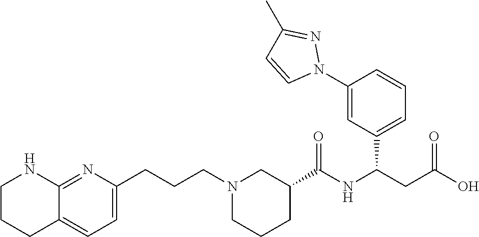 [SMILES: Cc1ccn(-c2cccc([C@H](CC(=O)O)NC(=O)[C@@H]3CCCN(CCCc4ccc5c(n4)NCCC5)C3)c2)n1] |
| 239 | 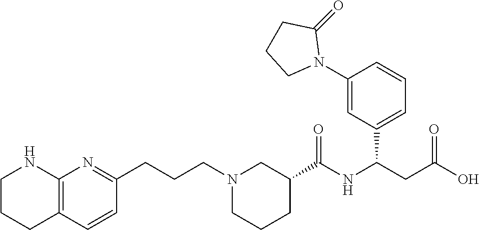 [SMILES: O=C(O)C[C@H](NC(=O)[C@@H]1CCCN(CCCc2ccc3c(n2)NCCC3)C1)c1cccc(N2CCCC2=O)c1] |
| 240 | 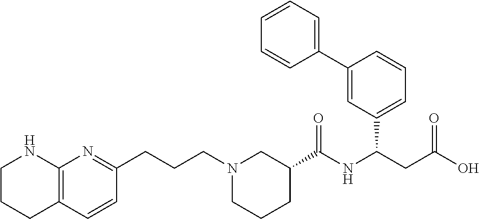 [SMILES: O=C(O)C[C@H](NC(=O)[C@@H]1CCCN(CCCc2ccc3c(n2)NCCC3)C1)c1cccc(-c2ccccc2)c1] |
| 241 | 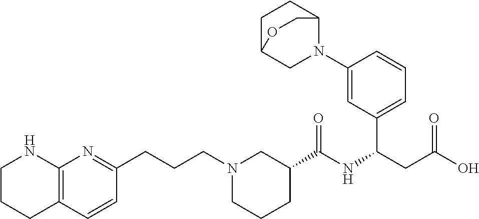 [SMILES: O=C(O)C[C@H](NC(=O)[C@@H]1CCCN(CCCc2ccc3c(n2)NCCC3)C1)c1cccc(N2CC3CCC2CO3)c1] |
| 241-A | 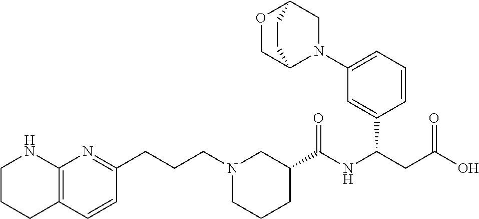 [SMILES: O=C(O)C[C@H](NC(=O)[C@@H]1CCCN(CCCc2ccc3c(n2)NCCC3)C1)c1cccc(N2C[C@H]3CC[C@@H]2CO3)c1] |
| 241-B | 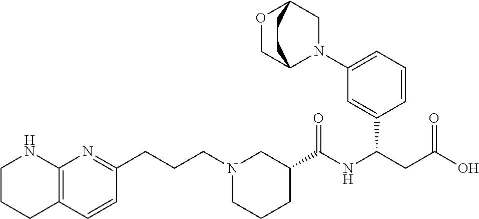 [SMILES: O=C(O)C[C@H](NC(=O)[C@@H]1CCCN(CCCc2ccc3c(n2)NCCC3)C1)c1cccc(N2C[C@@H]3CC[C@H]2CO3)c1] |
| 242 | 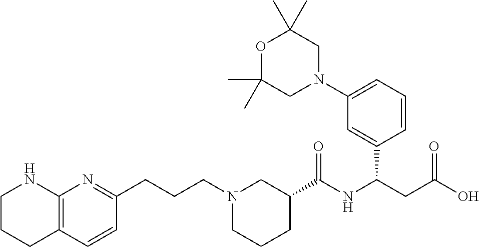 [SMILES: CC1(C)CN(c2cccc([C@H](CC(=O)O)NC(=O)[C@@H]3CCCN(CCCc4ccc5c(n4)NCCC5)C3)c2)CC(C)(C)O1] |
| 243 | 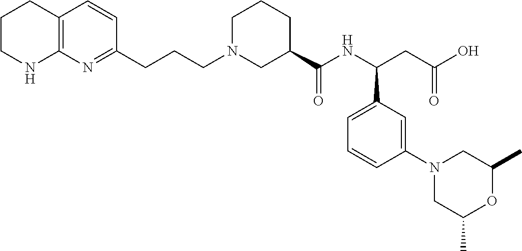 [SMILES: C[C@@H]1CN(c2cccc([C@H](CC(=O)O)NC(=O)[C@@H]3CCCN(CCCc4ccc5c(n4)NCCC5)C3)c2)C[C@@H](C)O1] |
| 244 | 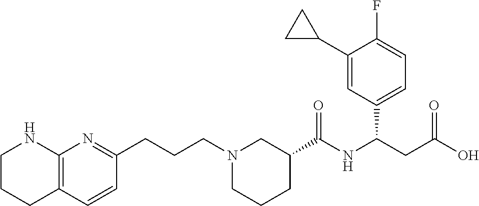 [SMILES: O=C(O)C[C@H](NC(=O)[C@@H]1CCCN(CCCc2ccc3c(n2)NCCC3)C1)c1ccc(F)c(C2CC2)c1] |
| 244-A | 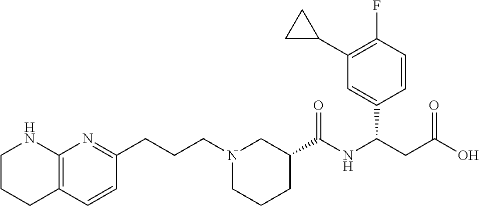 [SMILES: O=C(O)C[C@H](NC(=O)[C@@H]1CCCN(CCCc2ccc3c(n2)NCCC3)C1)c1ccc(F)c(C2CC2)c1] |
| 244-B | 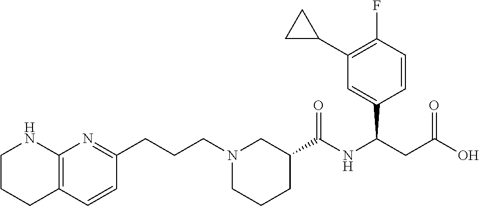 [SMILES: O=C(O)C[C@@H](NC(=O)[C@@H]1CCCN(CCCc2ccc3c(n2)NCCC3)C1)c1ccc(F)c(C2CC2)c1] |
| 245 | 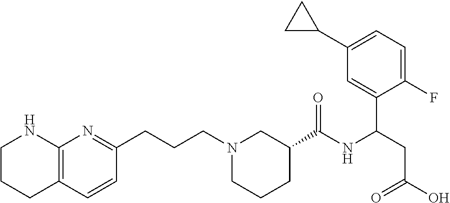 [SMILES: O=C(O)CC(NC(=O)[C@@H]1CCCN(CCCc2ccc3c(n2)NCCC3)C1)c1cc(C2CC2)ccc1F] |
| 245-A | 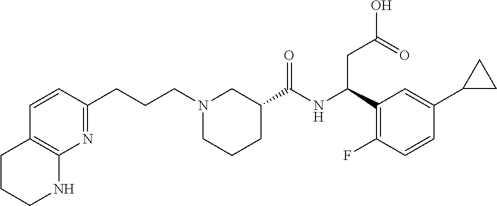 [SMILES: O=C(O)C[C@H](NC(=O)[C@@H]1CCCN(CCCc2ccc3c(n2)NCCC3)C1)c1cc(C2CC2)ccc1F] |
| 245-B | 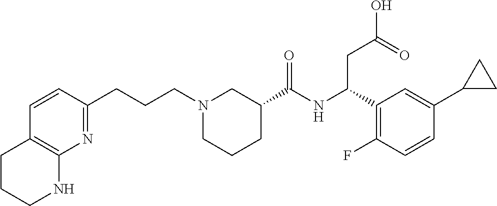 [SMILES: O=C(O)C[C@@H](NC(=O)[C@@H]1CCCN(CCCc2ccc3c(n2)NCCC3)C1)c1cc(C2CC2)ccc1F] |
| 246 | 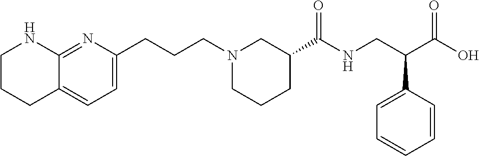 [SMILES: O=C(NC[C@@H](C(=O)O)c1ccccc1)[C@@H]1CCCN(CCCc2ccc3c(n2)NCCC3)C1] |
| 247 | 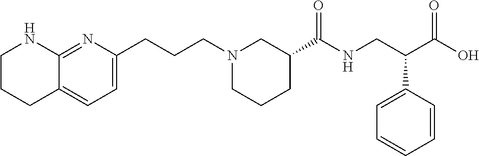 [SMILES: O=C(NC[C@H](C(=O)O)c1ccccc1)[C@@H]1CCCN(CCCc2ccc3c(n2)NCCC3)C1] |
| 248 | 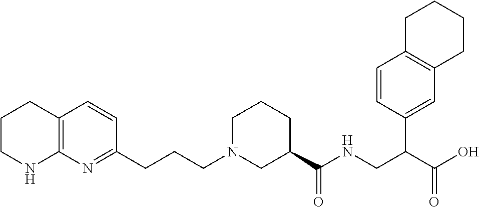 [SMILES: O=C(O)C(CNC(=O)[C@@H]1CCCN(CCCc2ccc3c(n2)NCCC3)C1)c1ccc2c(c1)CCCC2] |
| 248-A | 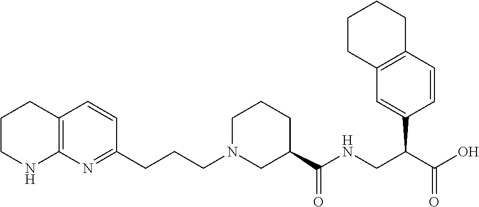 [SMILES: O=C(NC[C@H](C(=O)O)c1ccc2c(c1)CCCC2)[C@@H]1CCCN(CCCc2ccc3c(n2)NCCC3)C1] |
| 248-B | 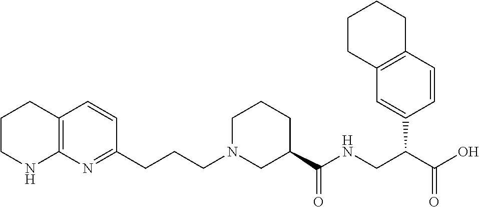 [SMILES: O=C(NC[C@@H](C(=O)O)c1ccc2c(c1)CCCC2)[C@@H]1CCCN(CCCc2ccc3c(n2)NCCC3)C1] |
| 249 | 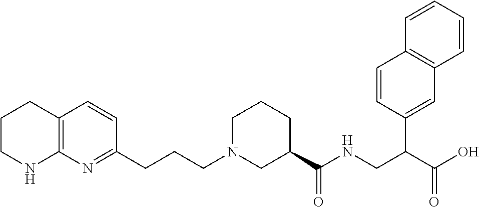 [SMILES: O=C(O)C(CNC(=O)[C@@H]1CCCN(CCCc2ccc3c(n2)NCCC3)C1)c1ccc2ccccc2c1] |
| 249-A | 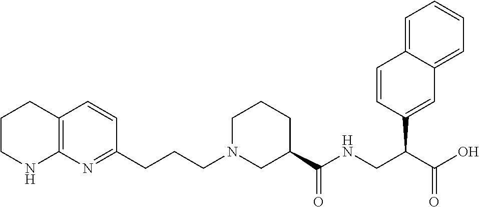 [SMILES: O=C(NC[C@H](C(=O)O)c1ccc2ccccc2c1)[C@@H]1CCCN(CCCc2ccc3c(n2)NCCC3)C1] |
| 249-B | 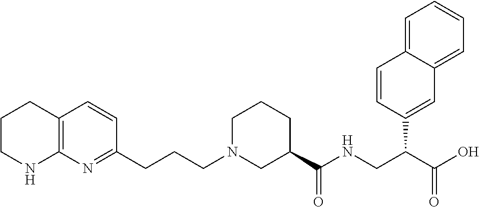 [SMILES: O=C(NC[C@@H](C(=O)O)c1ccc2ccccc2c1)[C@@H]1CCCN(CCCc2ccc3c(n2)NCCC3)C1] |
| 250 | 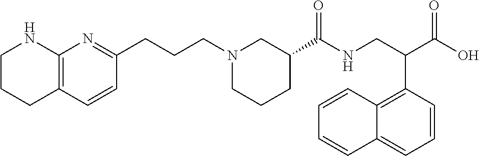 [SMILES: O=C(O)C(CNC(=O)[C@@H]1CCCN(CCCc2ccc3c(n2)NCCC3)C1)c1cccc2ccccc12] |
| 250-A | 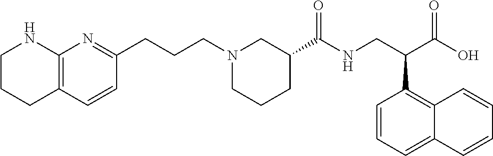 [SMILES: O=C(NC[C@@H](C(=O)O)c1cccc2ccccc12)[C@@H]1CCCN(CCCc2ccc3c(n2)NCCC3)C1] |
| 250-B | 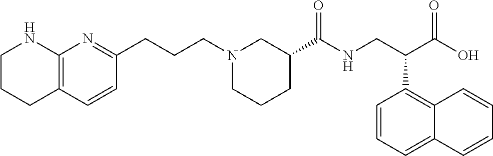 [SMILES: O=C(NC[C@H](C(=O)O)c1cccc2ccccc12)[C@@H]1CCCN(CCCc2ccc3c(n2)NCCC3)C1] |
| 251 | 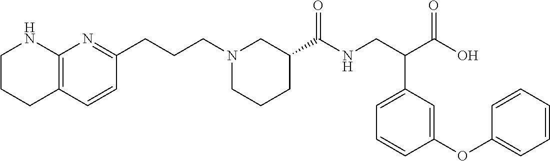 [SMILES: O=C(O)C(CNC(=O)[C@@H]1CCCN(CCCc2ccc3c(n2)NCCC3)C1)c1cccc(Oc2ccccc2)c1] |
| 251-A | 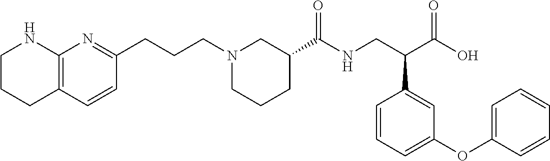 [SMILES: O=C(NC[C@@H](C(=O)O)c1cccc(Oc2ccccc2)c1)[C@@H]1CCCN(CCCc2ccc3c(n2)NCCC3)C1] |
| 251-B | 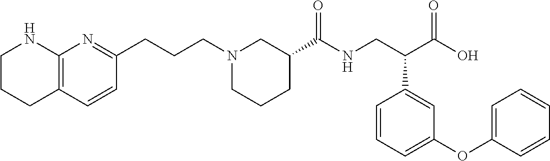 [SMILES: O=C(NC[C@H](C(=O)O)c1cccc(Oc2ccccc2)c1)[C@@H]1CCCN(CCCc2ccc3c(n2)NCCC3)C1] |
| 252 | 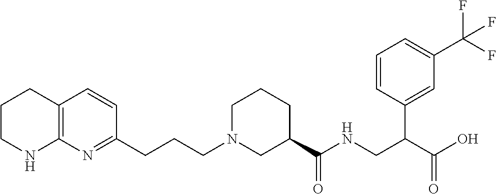 [SMILES: O=C(O)C(CNC(=O)[C@@H]1CCCN(CCCc2ccc3c(n2)NCCC3)C1)c1cccc(C(F)(F)F)c1] |
| 252-A | 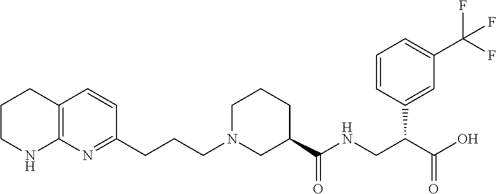 [SMILES: O=C(NC[C@@H](C(=O)O)c1cccc(C(F)(F)F)c1)[C@@H]1CCCN(CCCc2ccc3c(n2)NCCC3)C1] |
| 252-B | 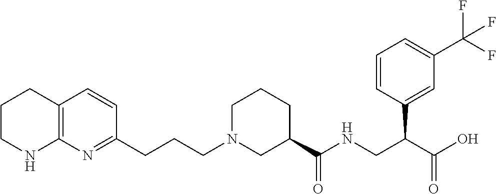 [SMILES: O=C(NC[C@H](C(=O)O)c1cccc(C(F)(F)F)c1)[C@@H]1CCCN(CCCc2ccc3c(n2)NCCC3)C1] |
| 253 | 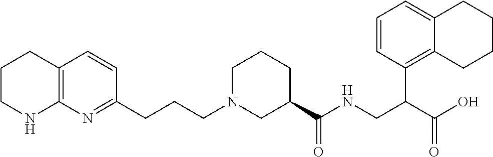 [SMILES: O=C(O)C(CNC(=O)[C@@H]1CCCN(CCCc2ccc3c(n2)NCCC3)C1)c1cccc2c1CCCC2] |
| 253-A | 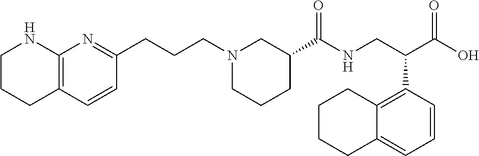 [SMILES: O=C(NC[C@H](C(=O)O)c1cccc2c1CCCC2)[C@@H]1CCCN(CCCc2ccc3c(n2)NCCC3)C1] |
| 253-B | 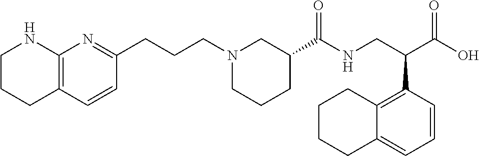 [SMILES: O=C(NC[C@@H](C(=O)O)c1cccc2c1CCCC2)[C@@H]1CCCN(CCCc2ccc3c(n2)NCCC3)C1] |
| 254 | 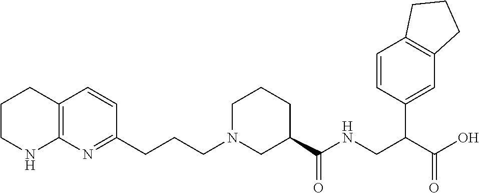 [SMILES: O=C(O)C(CNC(=O)[C@@H]1CCCN(CCCc2ccc3c(n2)NCCC3)C1)c1ccc2c(c1)CCC2] |
| 254-A | 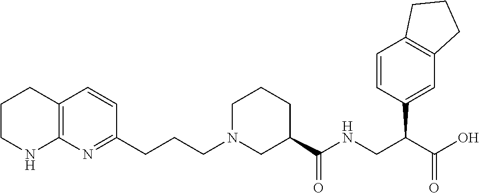 [SMILES: O=C(NC[C@H](C(=O)O)c1ccc2c(c1)CCC2)[C@@H]1CCCN(CCCc2ccc3c(n2)NCCC3)C1] |
| 254-B | 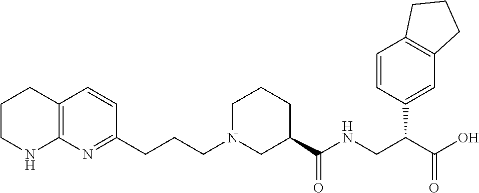 [SMILES: O=C(NC[C@@H](C(=O)O)c1ccc2c(c1)CCC2)[C@@H]1CCCN(CCCc2ccc3c(n2)NCCC3)C1] |
| 255 | 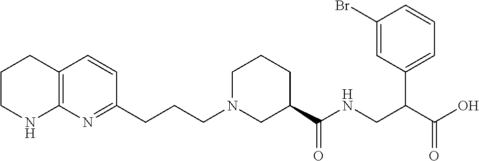 [SMILES: O=C(O)C(CNC(=O)[C@@H]1CCCN(CCCc2ccc3c(n2)NCCC3)C1)c1cccc(Br)c1] |
| 256 | 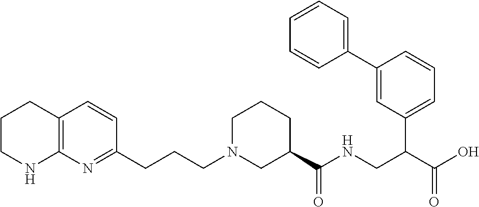 [SMILES: O=C(O)C(CNC(=O)[C@@H]1CCCN(CCCc2ccc3c(n2)NCCC3)C1)c1cccc(-c2ccccc2)c1] |
| 256-A | 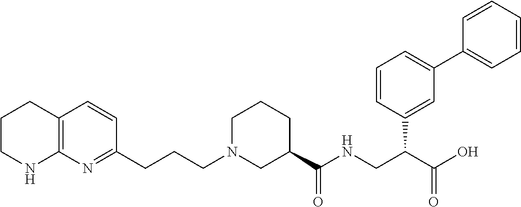 [SMILES: O=C(NC[C@@H](C(=O)O)c1cccc(-c2ccccc2)c1)[C@@H]1CCCN(CCCc2ccc3c(n2)NCCC3)C1] |
| 256-B | 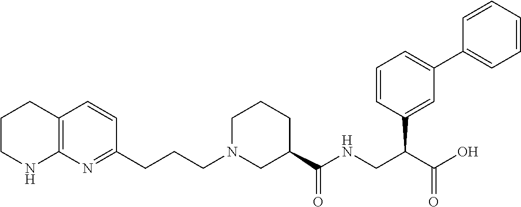 [SMILES: O=C(NC[C@H](C(=O)O)c1cccc(-c2ccccc2)c1)[C@@H]1CCCN(CCCc2ccc3c(n2)NCCC3)C1] |
| 257 | 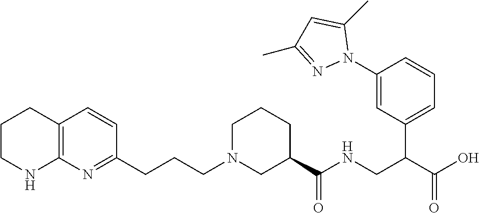 [SMILES: Cc1cc(C)n(-c2cccc(C(CNC(=O)[C@@H]3CCCN(CCCc4ccc5c(n4)NCCC5)C3)C(=O)O)c2)n1] |
| 257-A | 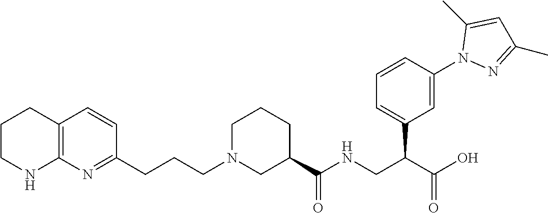 [SMILES: Cc1cc(C)n(-c2cccc([C@H](CNC(=O)[C@@H]3CCCN(CCCc4ccc5c(n4)NCCC5)C3)C(=O)O)c2)n1] |
| 257-B | 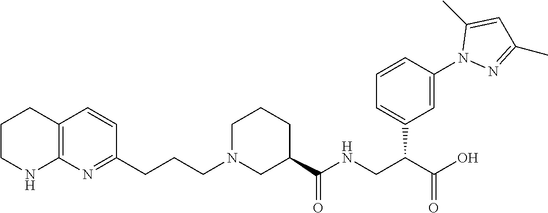 [SMILES: Cc1cc(C)n(-c2cccc([C@@H](CNC(=O)[C@@H]3CCCN(CCCc4ccc5c(n4)NCCC5)C3)C(=O)O)c2)n1] |
| 258 | 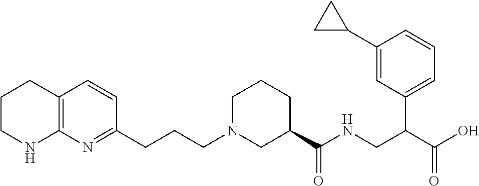 [SMILES: O=C(O)C(CNC(=O)[C@@H]1CCCN(CCCc2ccc3c(n2)NCCC3)C1)c1cccc(C2CC2)c1] |
| 258-A | 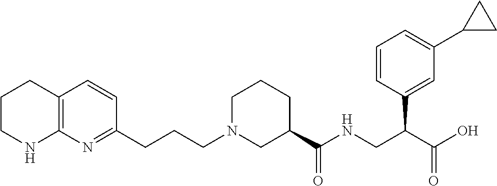 [SMILES: O=C(NC[C@H](C(=O)O)c1cccc(C2CC2)c1)[C@@H]1CCCN(CCCc2ccc3c(n2)NCCC3)C1] |
| 258-B | 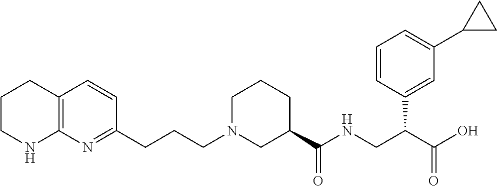 [SMILES: O=C(NC[C@@H](C(=O)O)c1cccc(C2CC2)c1)[C@@H]1CCCN(CCCc2ccc3c(n2)NCCC3)C1] |
| 259 | 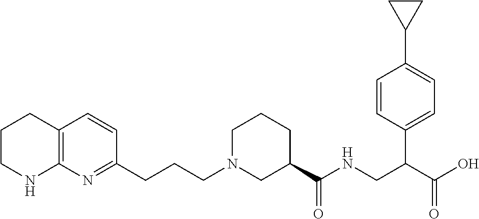 [SMILES: O=C(O)C(CNC(=O)[C@@H]1CCCN(CCCc2ccc3c(n2)NCCC3)C1)c1ccc(C2CC2)cc1] |
| 259-A | 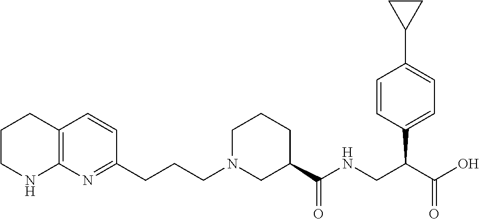 [SMILES: O=C(NC[C@H](C(=O)O)c1ccc(C2CC2)cc1)[C@@H]1CCCN(CCCc2ccc3c(n2)NCCC3)C1] |
| 259-B | 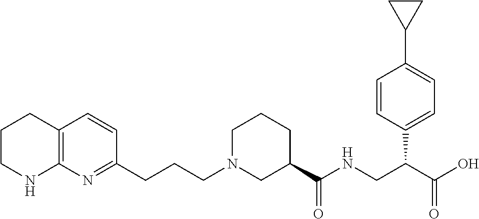 [SMILES: O=C(NC[C@@H](C(=O)O)c1ccc(C2CC2)cc1)[C@@H]1CCCN(CCCc2ccc3c(n2)NCCC3)C1] |
| 260 | 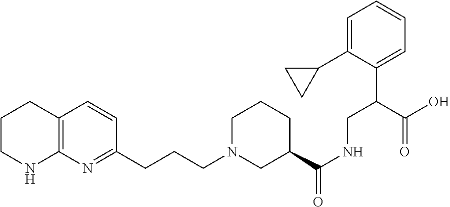 [SMILES: O=C(O)C(CNC(=O)[C@@H]1CCCN(CCCc2ccc3c(n2)NCCC3)C1)c1ccccc1C1CC1] |
| 260-A | 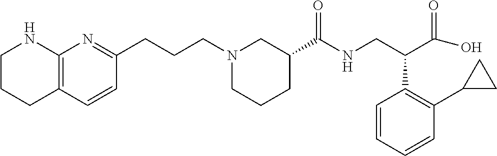 [SMILES: O=C(NC[C@H](C(=O)O)c1ccccc1C1CC1)[C@@H]1CCCN(CCCc2ccc3c(n2)NCCC3)C1] |
| 260-B | 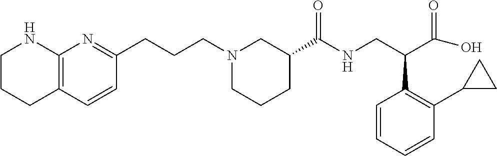 [SMILES: O=C(NC[C@@H](C(=O)O)c1ccccc1C1CC1)[C@@H]1CCCN(CCCc2ccc3c(n2)NCCC3)C1] |
| 261 | 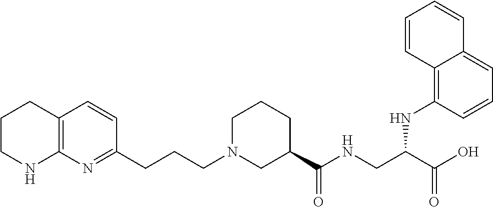 [SMILES: O=C(NC[C@H](Nc1cccc2ccccc12)C(=O)O)[C@@H]1CCCN(CCCc2ccc3c(n2)NCCC3)C1] |
| 262 | 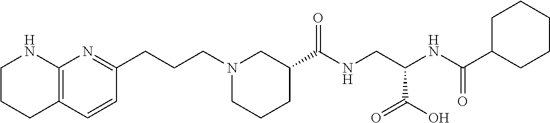 [SMILES: O=C(N[C@@H](CNC(=O)[C@@H]1CCCN(CCCc2ccc3c(n2)NCCC3)C1)C(=O)O)C1CCCCC1] |
| 263 | 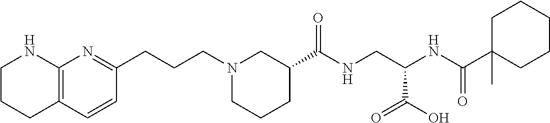 [SMILES: CC1(C(=O)N[C@@H](CNC(=O)[C@@H]2CCCN(CCCc3ccc4c(n3)NCCC4)C2)C(=O)O)CCCCC1] |
| 264 | 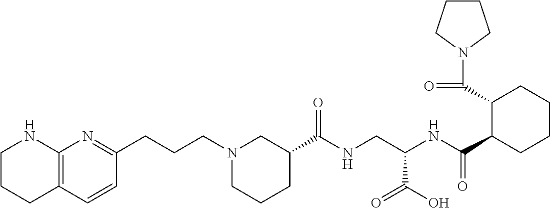 [SMILES: O=C(NC[C@H](NC(=O)[C@@H]1CCCC[C@H]1C(=O)N1CCCC1)C(=O)O)[C@@H]1CCCN(CCCc2ccc3c(n2)NCCC3)C1] |
| 265 | 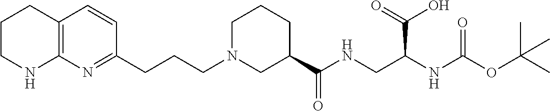 [SMILES: CC(C)(C)OC(=O)N[C@@H](CNC(=O)[C@@H]1CCCN(CCCc2ccc3c(n2)NCCC3)C1)C(=O)O] |
| 266 | 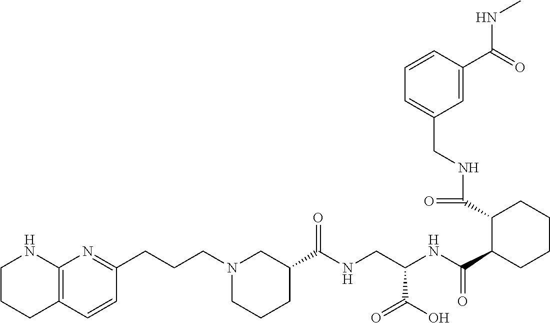 [SMILES: CNC(=O)c1cccc(CNC(=O)[C@@H]2CCCC[C@H]2C(=O)N[C@@H](CNC(=O)[C@@H]2CCCN(CCCc3ccc4c(n3)NCCC4)C2)C(=O)O)c1] |
| 267 | 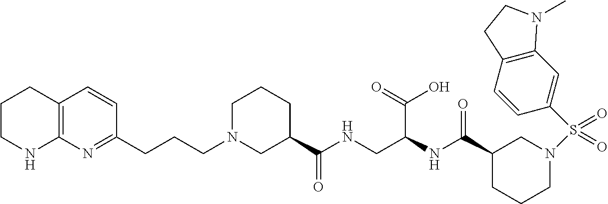 [SMILES: CN1CCc2ccc(S(=O)(=O)N3CCC[C@@H](C(=O)N[C@@H](CNC(=O)[C@@H]4CCCN(CCCc5ccc6c(n5)NCCC6)C4)C(=O)O)C3)cc21] |
| 268 | 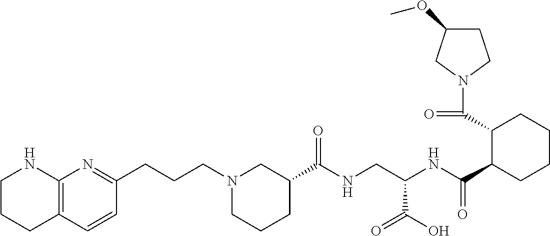 [SMILES: CO[C@H]1CCN(C(=O)[C@@H]2CCCC[C@H]2C(=O)N[C@@H](CNC(=O)[C@@H]2CCCN(CCCc3ccc4c(n3)NCCC4)C2)C(=O)O)C1] |
| 269 | 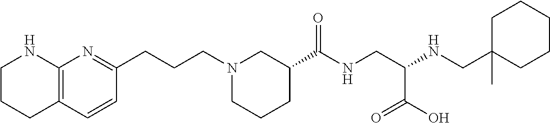 [SMILES: CC1(CN[C@@H](CNC(=O)[C@@H]2CCCN(CCCc3ccc4c(n3)NCCC4)C2)C(=O)O)CCCCC1] |
| 270 | 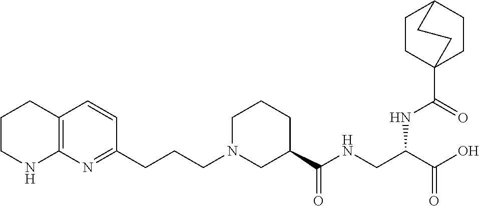 [SMILES: O=C(NC[C@H](NC(=O)C12CCC(CC1)CC2)C(=O)O)[C@@H]1CCCN(CCCc2ccc3c(n2)NCCC3)C1] |
| 271 | 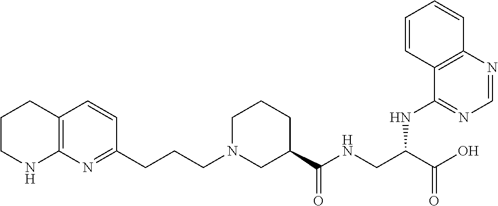 [SMILES: O=C(NC[C@H](Nc1ncnc2ccccc12)C(=O)O)[C@@H]1CCCN(CCCc2ccc3c(n2)NCCC3)C1] |
| 272 | 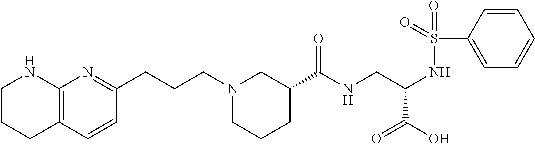 [SMILES: O=C(NC[C@H](NS(=O)(=O)c1ccccc1)C(=O)O)[C@@H]1CCCN(CCCc2ccc3c(n2)NCCC3)C1] |
| 273 | 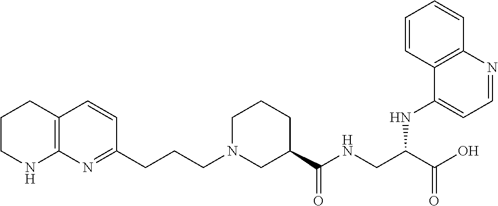 [SMILES: O=C(NC[C@H](Nc1ccnc2ccccc12)C(=O)O)[C@@H]1CCCN(CCCc2ccc3c(n2)NCCC3)C1] |
| 274 | 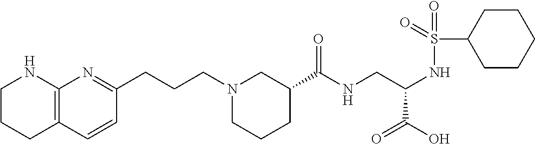 [SMILES: O=C(NC[C@H](NS(=O)(=O)C1CCCCC1)C(=O)O)[C@@H]1CCCN(CCCc2ccc3c(n2)NCCC3)C1] |
| 275 | 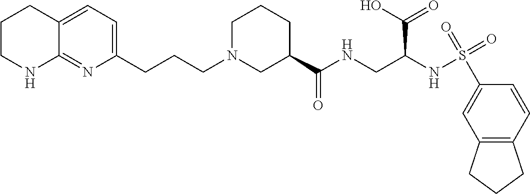 [SMILES: O=C(NC[C@H](NS(=O)(=O)c1ccc2c(c1)CCC2)C(=O)O)[C@@H]1CCCN(CCCc2ccc3c(n2)NCCC3)C1] |
| 276 | 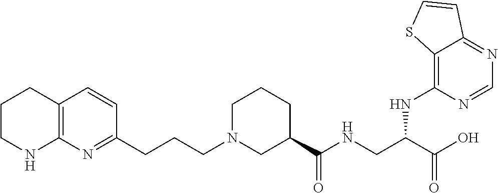 [SMILES: O=C(NC[C@H](Nc1ncnc2ccsc12)C(=O)O)[C@@H]1CCCN(CCCc2ccc3c(n2)NCCC3)C1] |
| 277 | 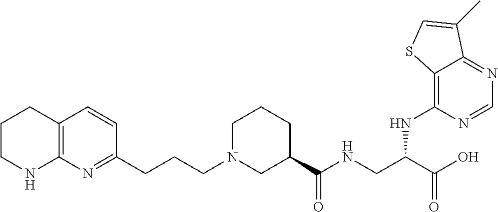 [SMILES: Cc1csc2c(N[C@@H](CNC(=O)[C@@H]3CCCN(CCCc4ccc5c(n4)NCCC5)C3)C(=O)O)ncnc12] |
| 278 | 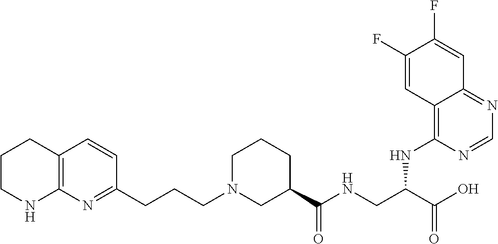 [SMILES: O=C(NC[C@H](Nc1ncnc2cc(F)c(F)cc12)C(=O)O)[C@@H]1CCCN(CCCc2ccc3c(n2)NCCC3)C1] |
| 279 | 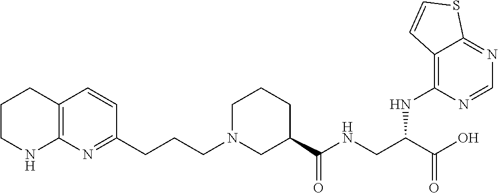 [SMILES: O=C(NC[C@H](Nc1ncnc2sccc12)C(=O)O)[C@@H]1CCCN(CCCc2ccc3c(n2)NCCC3)C1] |
| 280 | 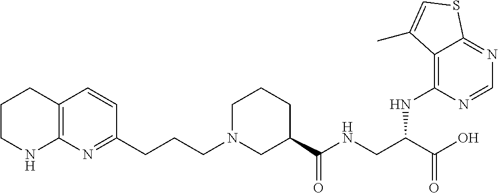 [SMILES: Cc1csc2ncnc(N[C@@H](CNC(=O)[C@@H]3CCCN(CCCc4ccc5c(n4)NCCC5)C3)C(=O)O)c12] |
| 281 | 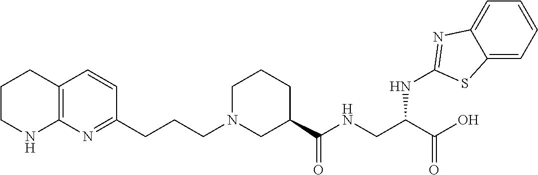 [SMILES: O=C(NC[C@H](Nc1nc2ccccc2s1)C(=O)O)[C@@H]1CCCN(CCCc2ccc3c(n2)NCCC3)C1] |
| 282 | 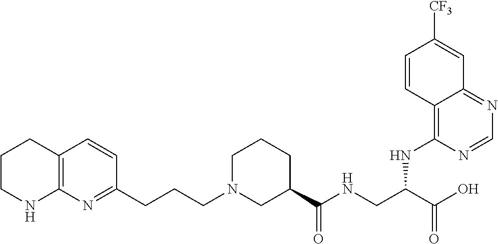 [SMILES: O=C(NC[C@H](Nc1ncnc2cc(C(F)(F)F)ccc12)C(=O)O)[C@@H]1CCCN(CCCc2ccc3c(n2)NCCC3)C1] |
| 283 | 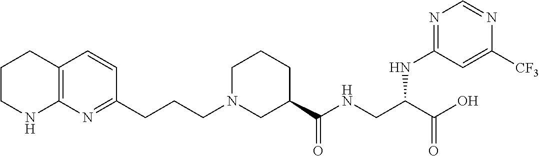 [SMILES: O=C(NC[C@H](Nc1cc(C(F)(F)F)ncn1)C(=O)O)[C@@H]1CCCN(CCCc2ccc3c(n2)NCCC3)C1] |
| 284 | 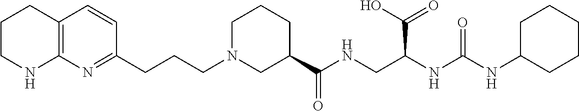 [SMILES: O=C(NC1CCCCC1)N[C@@H](CNC(=O)[C@@H]1CCCN(CCCc2ccc3c(n2)NCCC3)C1)C(=O)O] |
| 285 | 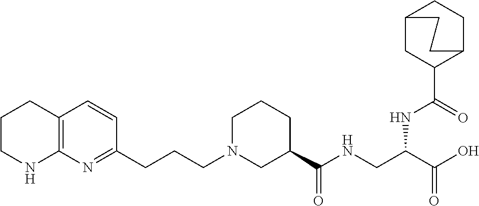 [SMILES: O=C(N[C@@H](CNC(=O)[C@@H]1CCCN(CCCc2ccc3c(n2)NCCC3)C1)C(=O)O)C1CC2CCC1CC2] |
| 286 | 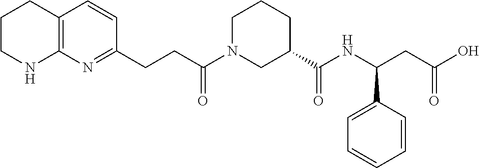 [SMILES: O=C(O)C[C@H](NC(=O)[C@H]1CCCN(C(=O)CCc2ccc3c(n2)NCCC3)C1)c1ccccc1] |
| 287 | 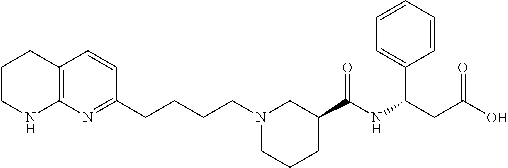 [SMILES: O=C(O)C[C@H](NC(=O)[C@H]1CCCN(CCCCc2ccc3c(n2)NCCC3)C1)c1ccccc1] |
| 288 | 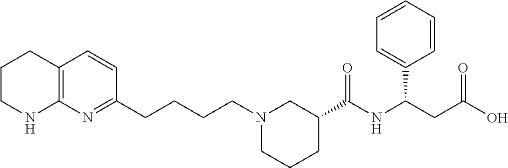 [SMILES: O=C(O)C[C@H](NC(=O)[C@@H]1CCCN(CCCCc2ccc3c(n2)NCCC3)C1)c1ccccc1] |
| 289 | 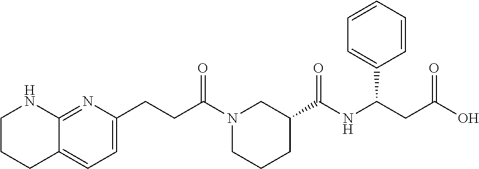 [SMILES: O=C(O)C[C@H](NC(=O)[C@@H]1CCCN(C(=O)CCc2ccc3c(n2)NCCC3)C1)c1ccccc1] |
| 290 | 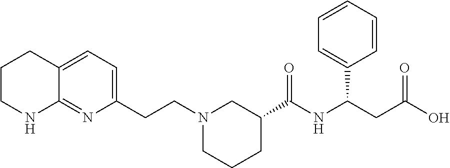 [SMILES: O=C(O)C[C@H](NC(=O)[C@@H]1CCCN(CCc2ccc3c(n2)NCCC3)C1)c1ccccc1] |
| 291 | 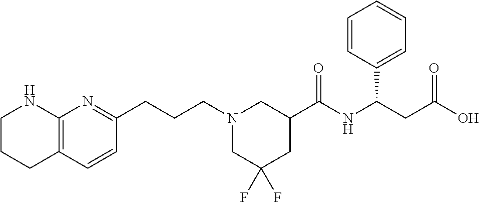 [SMILES: O=C(O)C[C@H](NC(=O)C1CN(CCCc2ccc3c(n2)NCCC3)CC(F)(F)C1)c1ccccc1] |
| 291-A | 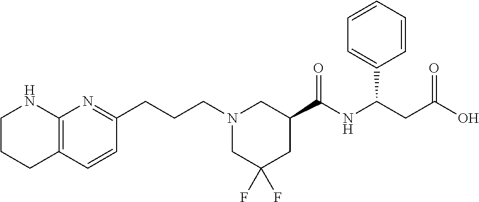 [SMILES: O=C(O)C[C@H](NC(=O)[C@@H]1CN(CCCc2ccc3c(n2)NCCC3)CC(F)(F)C1)c1ccccc1] |
| 291-B | 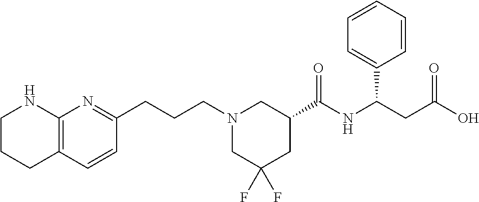 [SMILES: O=C(O)C[C@H](NC(=O)[C@H]1CN(CCCc2ccc3c(n2)NCCC3)CC(F)(F)C1)c1ccccc1] |
| 292 | 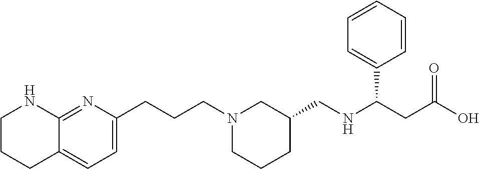 [SMILES: O=C(O)C[C@H](NC[C@@H]1CCCN(CCCc2ccc3c(n2)NCCC3)C1)c1ccccc1] |
| 293 | 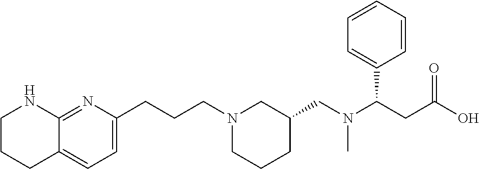 [SMILES: CN(C[C@@H]1CCCN(CCCc2ccc3c(n2)NCCC3)C1)[C@@H](CC(=O)O)c1ccccc1] |
| 294 | 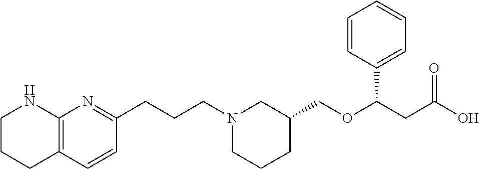 [SMILES: O=C(O)C[C@H](OC[C@@H]1CCCN(CCCc2ccc3c(n2)NCCC3)C1)c1ccccc1] |
| 295 | 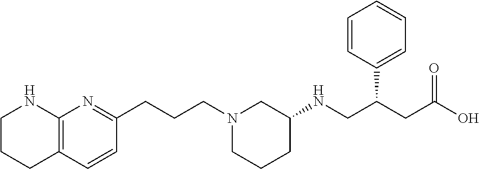 [SMILES: O=C(O)C[C@H](CN[C@@H]1CCCN(CCCc2ccc3c(n2)NCCC3)C1)c1ccccc1] |
| 296 | 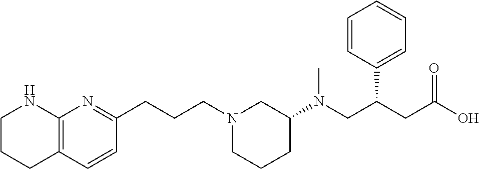 [SMILES: CN(C[C@@H](CC(=O)O)c1ccccc1)[C@@H]1CCCN(CCCc2ccc3c(n2)NCCC3)C1] |
| 297 | 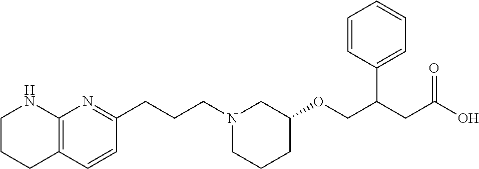 [SMILES: O=C(O)CC(CO[C@@H]1CCCN(CCCc2ccc3c(n2)NCCC3)C1)c1ccccc1] |
| 297-A | 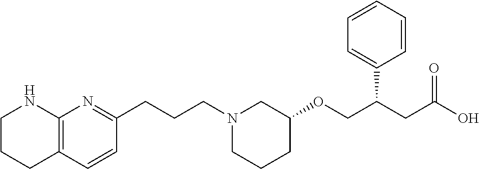 [SMILES: O=C(O)C[C@H](CO[C@@H]1CCCN(CCCc2ccc3c(n2)NCCC3)C1)c1ccccc1] |
| 297-B | 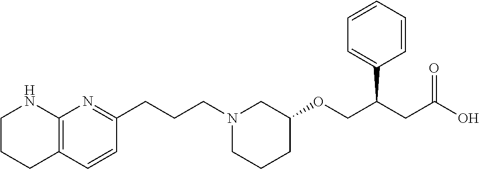 [SMILES: O=C(O)C[C@@H](CO[C@@H]1CCCN(CCCc2ccc3c(n2)NCCC3)C1)c1ccccc1] |
| 298 | 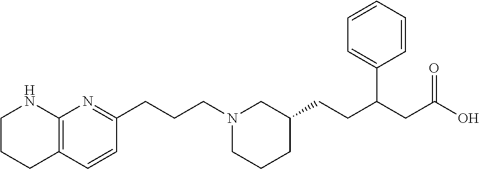 [SMILES: O=C(O)CC(CC[C@@H]1CCCN(CCCc2ccc3c(n2)NCCC3)C1)c1ccccc1] |
| 298-A | 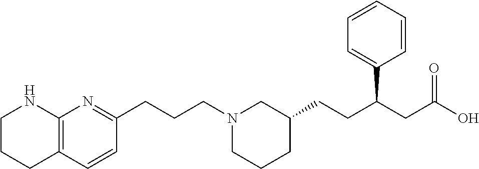 [SMILES: O=C(O)C[C@@H](CC[C@@H]1CCCN(CCCc2ccc3c(n2)NCCC3)C1)c1ccccc1] |
| 298-B | 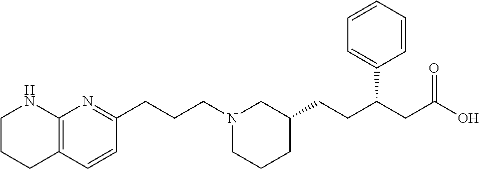 [SMILES: O=C(O)C[C@H](CC[C@@H]1CCCN(CCCc2ccc3c(n2)NCCC3)C1)c1ccccc1] |
| 299 | 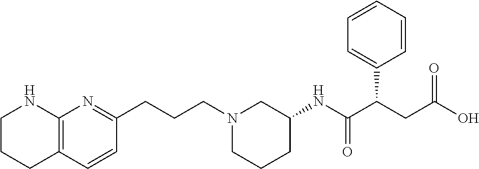 [SMILES: O=C(O)C[C@H](C(=O)N[C@@H]1CCCN(CCCc2ccc3c(n2)NCCC3)C1)c1ccccc1] |
| 300 | 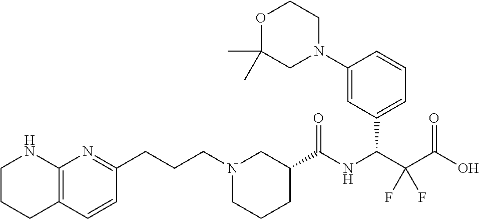 [SMILES: CC1(C)CN(c2cccc([C@@H](NC(=O)[C@@H]3CCCN(CCCc4ccc5c(n4)NCCC5)C3)C(F)(F)C(=O)O)c2)CCO1] |
| 301 | 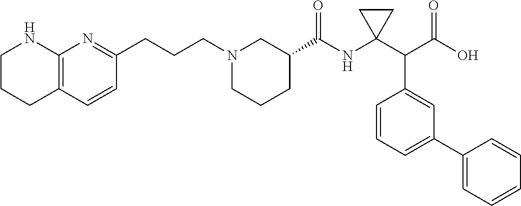 [SMILES: O=C(O)C(c1cccc(-c2ccccc2)c1)C1(NC(=O)[C@@H]2CCCN(CCCc3ccc4c(n3)NCCC4)C2)CC1] |
| 301-A | 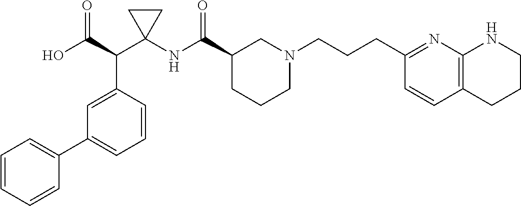 [SMILES: O=C(NC1([C@H](C(=O)O)c2cccc(-c3ccccc3)c2)CC1)[C@@H]1CCCN(CCCc2ccc3c(n2)NCCC3)C1] |
| 301-B | 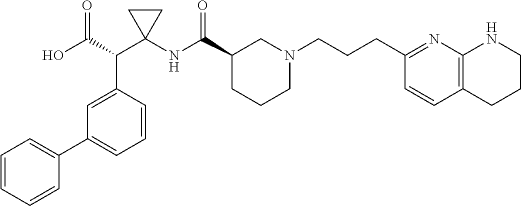 [SMILES: O=C(NC1([C@@H](C(=O)O)c2cccc(-c3ccccc3)c2)CC1)[C@@H]1CCCN(CCCc2ccc3c(n2)NCCC3)C1] |
| 302 | 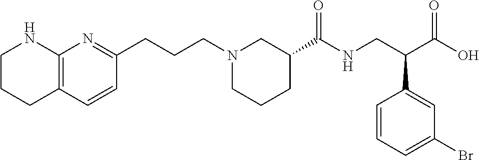 [SMILES: O=C(NC[C@@H](C(=O)O)c1cccc(Br)c1)[C@@H]1CCCN(CCCc2ccc3c(n2)NCCC3)C1] |
| 303 | 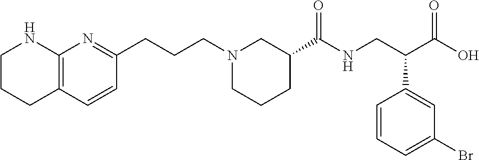 [SMILES: O=C(NC[C@H](C(=O)O)c1cccc(Br)c1)[C@@H]1CCCN(CCCc2ccc3c(n2)NCCC3)C1] |
| 304 | 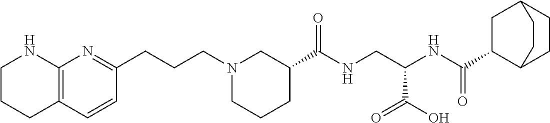 [SMILES: O=C(NC[C@H](NC(=O)[C@@H]1CC2CCC1CC2)C(=O)O)[C@@H]1CCCN(CCCc2ccc3c(n2)NCCC3)C1] |
| 305 | 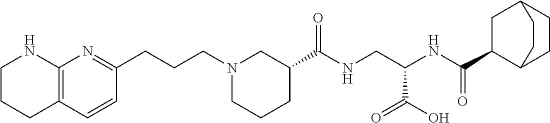 [SMILES: O=C(NC[C@H](NC(=O)[C@H]1CC2CCC1CC2)C(=O)O)[C@@H]1CCCN(CCCc2ccc3c(n2)NCCC3)C1] |
| 306 | 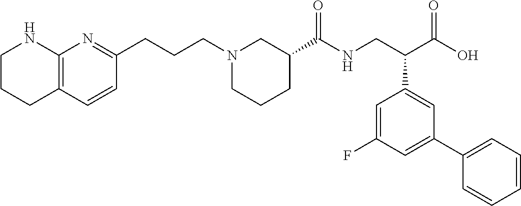 [SMILES: O=C(NC[C@H](C(=O)O)c1cc(F)cc(-c2ccccc2)c1)[C@@H]1CCCN(CCCc2ccc3c(n2)NCCC3)C1] |
| 307 | 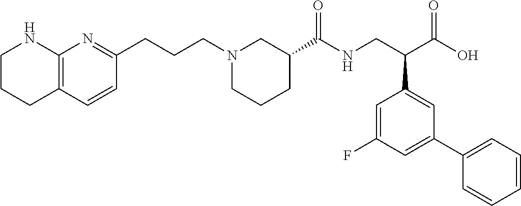 [SMILES: O=C(NC[C@@H](C(=O)O)c1cc(F)cc(-c2ccccc2)c1)[C@@H]1CCCN(CCCc2ccc3c(n2)NCCC3)C1] |
| 308 | 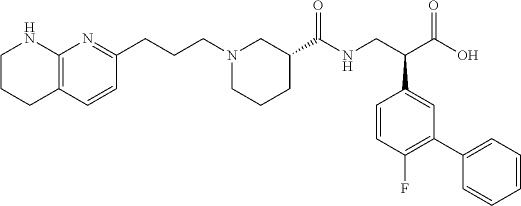 [SMILES: O=C(NC[C@@H](C(=O)O)c1ccc(F)c(-c2ccccc2)c1)[C@@H]1CCCN(CCCc2ccc3c(n2)NCCC3)C1] |
| 309 | 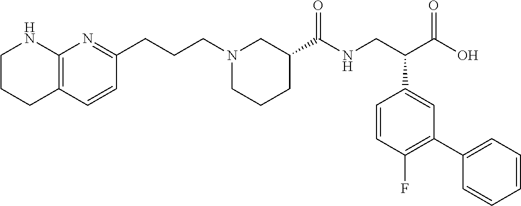 [SMILES: O=C(NC[C@H](C(=O)O)c1ccc(F)c(-c2ccccc2)c1)[C@@H]1CCCN(CCCc2ccc3c(n2)NCCC3)C1] |
| 310 | 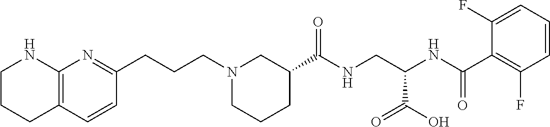 [SMILES: O=C(N[C@@H](CNC(=O)[C@@H]1CCCN(CCCc2ccc3c(n2)NCCC3)C1)C(=O)O)c1c(F)cccc1F] |
| 311 | 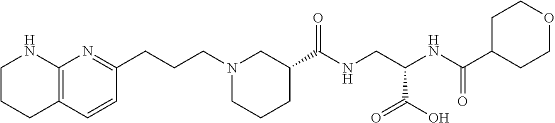 [SMILES: O=C(N[C@@H](CNC(=O)[C@@H]1CCCN(CCCc2ccc3c(n2)NCCC3)C1)C(=O)O)C1CCOCC1] |
| 312 | 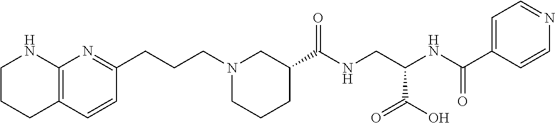 [SMILES: O=C(N[C@@H](CNC(=O)[C@@H]1CCCN(CCCc2ccc3c(n2)NCCC3)C1)C(=O)O)c1ccncc1] |
| 313 | 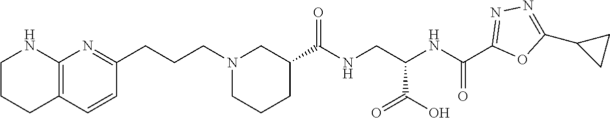 [SMILES: O=C(N[C@@H](CNC(=O)[C@@H]1CCCN(CCCc2ccc3c(n2)NCCC3)C1)C(=O)O)c1nnc(C2CC2)o1] |
| 314 | 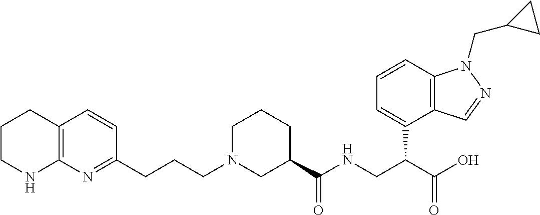 [SMILES: O=C(NC[C@@H](C(=O)O)c1cccc2c1cnn2CC1CC1)[C@@H]1CCCN(CCCc2ccc3c(n2)NCCC3)C1] |
| 315 | 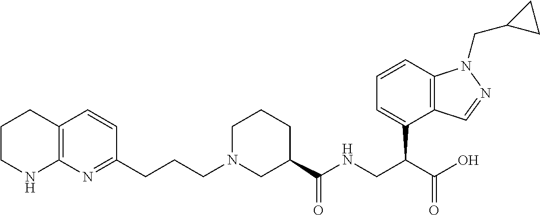 [SMILES: O=C(NC[C@H](C(=O)O)c1cccc2c1cnn2CC1CC1)[C@@H]1CCCN(CCCc2ccc3c(n2)NCCC3)C1] |
| 316 | 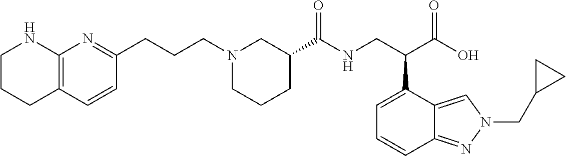 [SMILES: O=C(NC[C@@H](C(=O)O)c1cccc2nn(CC3CC3)cc12)[C@@H]1CCCN(CCCc2ccc3c(n2)NCCC3)C1] |
| 317 | 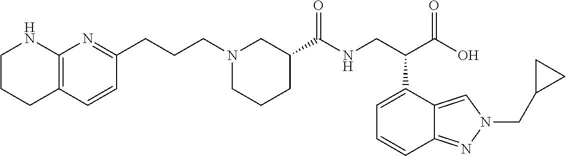 [SMILES: O=C(NC[C@H](C(=O)O)c1cccc2nn(CC3CC3)cc12)[C@@H]1CCCN(CCCc2ccc3c(n2)NCCC3)C1] |
| 318 | 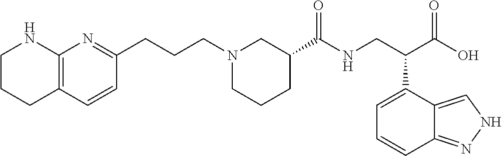 [SMILES: O=C(NC[C@H](C(=O)O)c1cccc2n[nH]cc12)[C@@H]1CCCN(CCCc2ccc3c(n2)NCCC3)C1] |
| 319 | 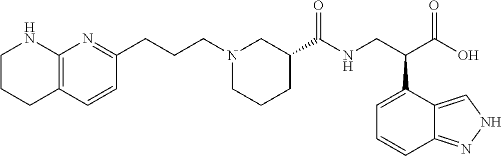 [SMILES: O=C(NC[C@@H](C(=O)O)c1cccc2n[nH]cc12)[C@@H]1CCCN(CCCc2ccc3c(n2)NCCC3)C1] |
| 320 | 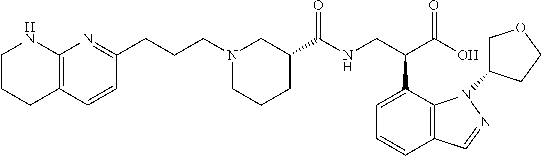 [SMILES: O=C(NC[C@@H](C(=O)O)c1cccc2cnn([C@H]3CCOC3)c12)[C@@H]1CCCN(CCCc2ccc3c(n2)NCCC3)C1] |
| 321 | 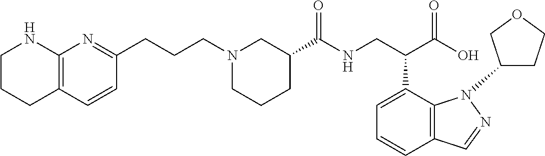 [SMILES: O=C(NC[C@H](C(=O)O)c1cccc2cnn([C@H]3CCOC3)c12)[C@@H]1CCCN(CCCc2ccc3c(n2)NCCC3)C1] |
| 322 | 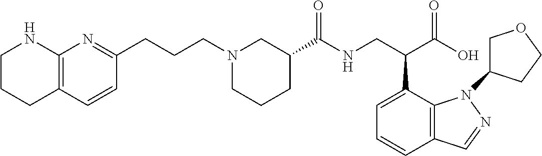 [SMILES: O=C(NC[C@@H](C(=O)O)c1cccc2cnn([C@@H]3CCOC3)c12)[C@@H]1CCCN(CCCc2ccc3c(n2)NCCC3)C1] |
| 323 | 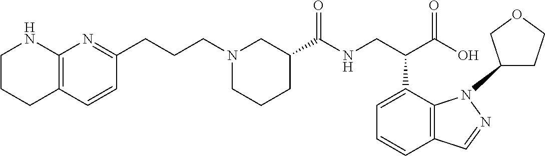 [SMILES: O=C(NC[C@H](C(=O)O)c1cccc2cnn([C@@H]3CCOC3)c12)[C@@H]1CCCN(CCCc2ccc3c(n2)NCCC3)C1] |
| 324 | 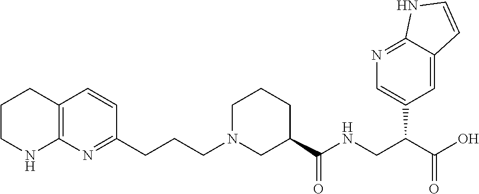 [SMILES: O=C(NC[C@@H](C(=O)O)c1cnc2[nH]ccc2c1)[C@@H]1CCCN(CCCc2ccc3c(n2)NCCC3)C1] |
| 325 | 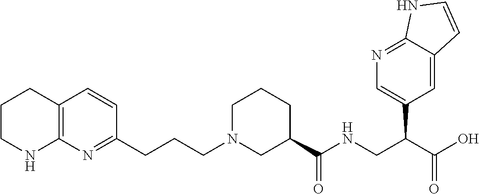 [SMILES: O=C(NC[C@H](C(=O)O)c1cnc2[nH]ccc2c1)[C@@H]1CCCN(CCCc2ccc3c(n2)NCCC3)C1] |
| 326 | 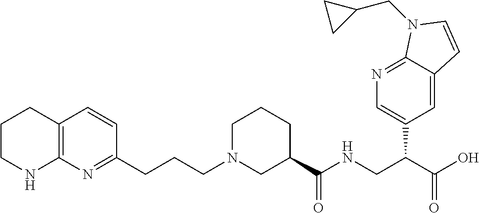 [SMILES: O=C(NC[C@@H](C(=O)O)c1cnc2c(ccn2CC2CC2)c1)[C@@H]1CCCN(CCCc2ccc3c(n2)NCCC3)C1] |
| 327 | 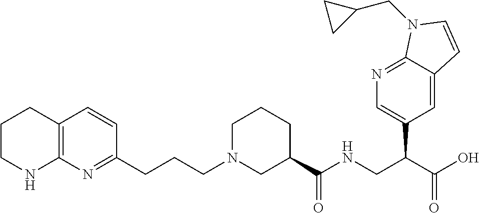 [SMILES: O=C(NC[C@H](C(=O)O)c1cnc2c(ccn2CC2CC2)c1)[C@@H]1CCCN(CCCc2ccc3c(n2)NCCC3)C1] |
| 328 | 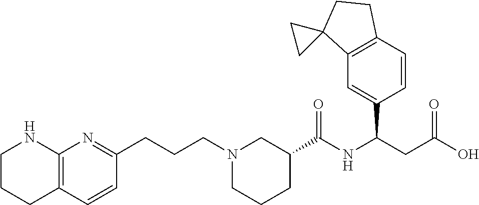 [SMILES: O=C(O)C[C@@H](NC(=O)[C@@H]1CCCN(CCCc2ccc3c(n2)NCCC3)C1)c1ccc2c(c1)C1(CC2)CC1] |
| 329 | 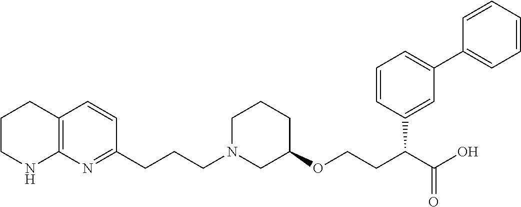 [SMILES: O=C(O)[C@H](CCO[C@@H]1CCCN(CCCc2ccc3c(n2)NCCC3)C1)c1cccc(-c2ccccc2)c1] |
| 330 | 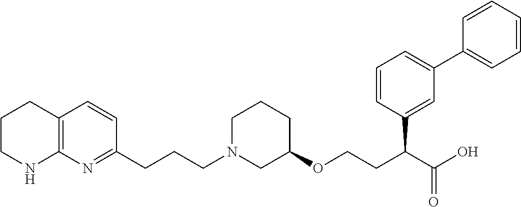 [SMILES: O=C(O)[C@@H](CCO[C@@H]1CCCN(CCCc2ccc3c(n2)NCCC3)C1)c1cccc(-c2ccccc2)c1] |
| 331 | 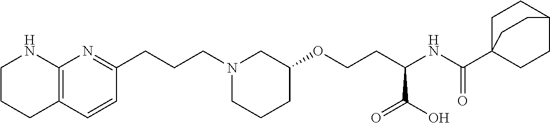 [SMILES: O=C(O)[C@@H](CCO[C@@H]1CCCN(CCCc2ccc3c(n2)NCCC3)C1)NC(=O)C12CCC(CC1)CC2] |
| 332 | 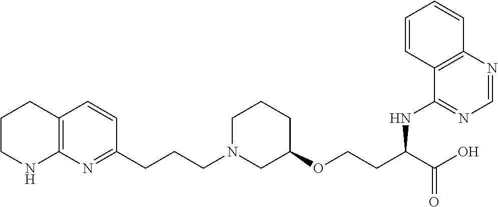 [SMILES: O=C(O)[C@@H](CCO[C@@H]1CCCN(CCCc2ccc3c(n2)NCCC3)C1)Nc1ncnc2ccccc12] |
| 333 | 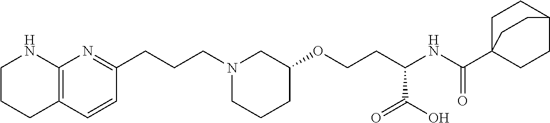 [SMILES: O=C(O)[C@H](CCO[C@@H]1CCCN(CCCc2ccc3c(n2)NCCC3)C1)NC(=O)C12CCC(CC1)CC2] |
| 334 | 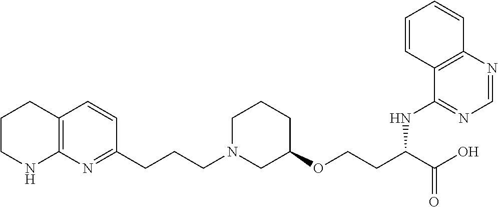 [SMILES: O=C(O)[C@H](CCO[C@@H]1CCCN(CCCc2ccc3c(n2)NCCC3)C1)Nc1ncnc2ccccc12] |
| 335 | 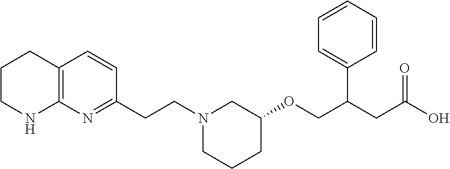 [SMILES: O=C(O)CC(CO[C@@H]1CCCN(CCc2ccc3c(n2)NCCC3)C1)c1ccccc1] |
| 336 | 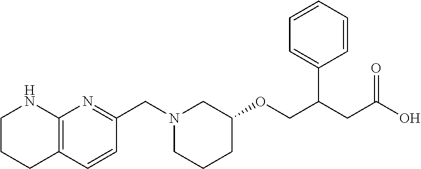 [SMILES: O=C(O)CC(CO[C@@H]1CCCN(Cc2ccc3c(n2)NCCC3)C1)c1ccccc1] |
| 337 | 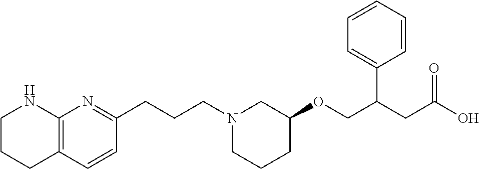 [SMILES: O=C(O)CC(CO[C@H]1CCCN(CCCc2ccc3c(n2)NCCC3)C1)c1ccccc1] |
| 338 | 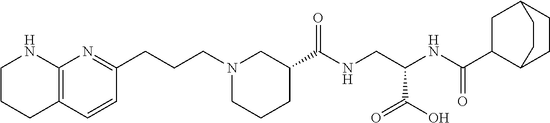 [SMILES: O=C(N[C@@H](CNC(=O)[C@@H]1CCCN(CCCc2ccc3c(n2)NCCC3)C1)C(=O)O)C1CC2CCC1CC2] |
| 339 | 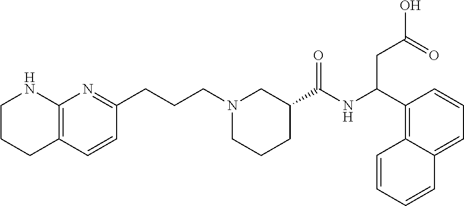 [SMILES: O=C(O)CC(NC(=O)[C@@H]1CCCN(CCCc2ccc3c(n2)NCCC3)C1)c1cccc2ccccc12] |
| 340 | 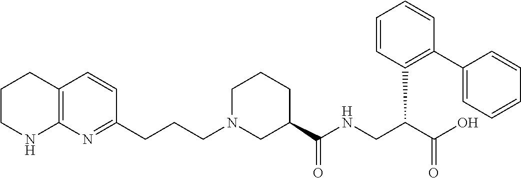 [SMILES: O=C(NC[C@@H](C(=O)O)c1ccccc1-c1ccccc1)[C@@H]1CCCN(CCCc2ccc3c(n2)NCCC3)C1] |
| 341 | 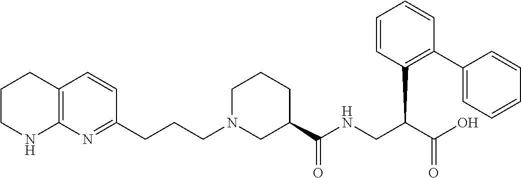 [SMILES: O=C(NC[C@H](C(=O)O)c1ccccc1-c1ccccc1)[C@@H]1CCCN(CCCc2ccc3c(n2)NCCC3)C1] |
| 342 | 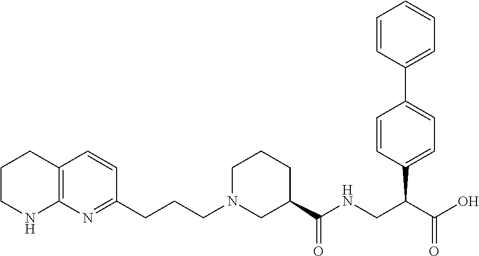 [SMILES: O=C(NC[C@H](C(=O)O)c1ccc(-c2ccccc2)cc1)[C@@H]1CCCN(CCCc2ccc3c(n2)NCCC3)C1] |
| 343 | 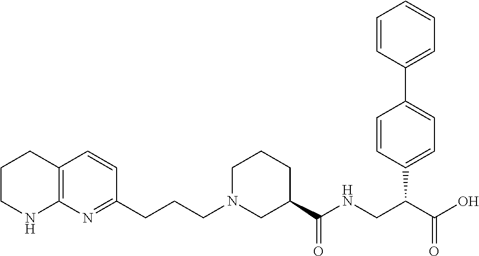 [SMILES: O=C(NC[C@@H](C(=O)O)c1ccc(-c2ccccc2)cc1)[C@@H]1CCCN(CCCc2ccc3c(n2)NCCC3)C1] |
| 344 | 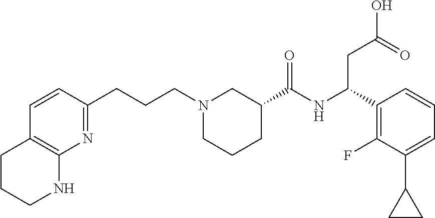 [SMILES: O=C(O)C[C@@H](NC(=O)[C@@H]1CCCN(CCCc2ccc3c(n2)NCCC3)C1)c1cccc(C2CC2)c1F] |
| 345 | 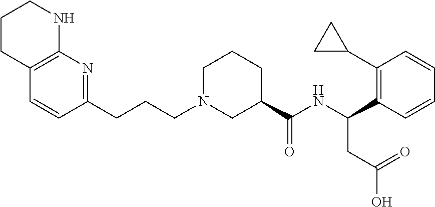 [SMILES: O=C(O)C[C@@H](NC(=O)[C@@H]1CCCN(CCCc2ccc3c(n2)NCCC3)C1)c1ccccc1C1CC1] |
| 346 | 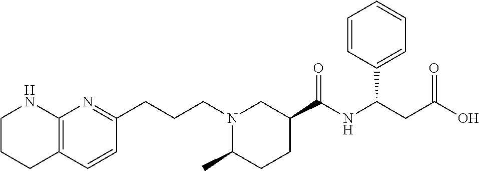 [SMILES: C[C@@H]1CC[C@H](C(=O)N[C@@H](CC(=O)O)c2ccccc2)CN1CCCc1ccc2c(n1)NCCC2] |
| 347 | 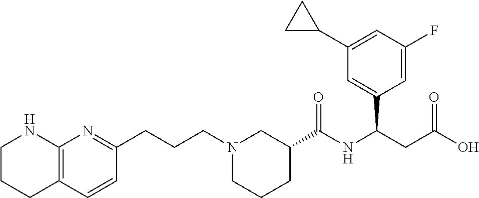 [SMILES: O=C(O)C[C@@H](NC(=O)[C@@H]1CCCN(CCCc2ccc3c(n2)NCCC3)C1)c1cc(F)cc(C2CC2)c1] |
| 348 | 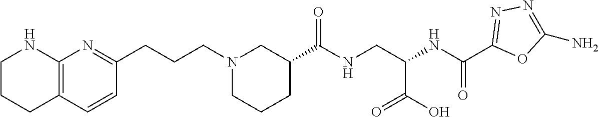 [SMILES: Nc1nnc(C(=O)N[C@@H](CNC(=O)[C@@H]2CCCN(CCCc3ccc4c(n3)NCCC4)C2)C(=O)O)o1] |
| 349 | 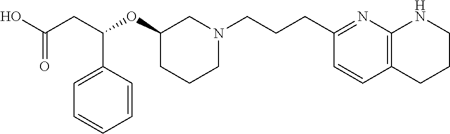 [SMILES: O=C(O)C[C@H](O[C@@H]1CCCN(CCCc2ccc3c(n2)NCCC3)C1)c1ccccc1] |
| 350 | 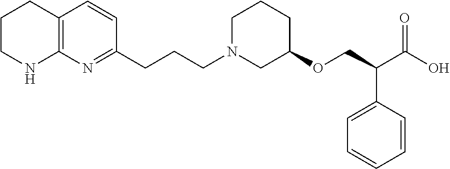 [SMILES: O=C(O)[C@H](CO[C@@H]1CCCN(CCCc2ccc3c(n2)NCCC3)C1)c1ccccc1] |
| 351 | 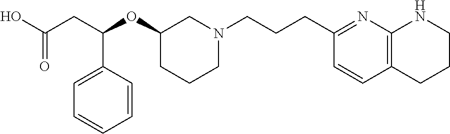 [SMILES: O=C(O)C[C@@H](O[C@@H]1CCCN(CCCc2ccc3c(n2)NCCC3)C1)c1ccccc1] |
| 352 | 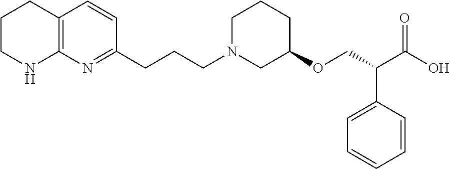 [SMILES: O=C(O)[C@@H](CO[C@@H]1CCCN(CCCc2ccc3c(n2)NCCC3)C1)c1ccccc1] |
| 353 | 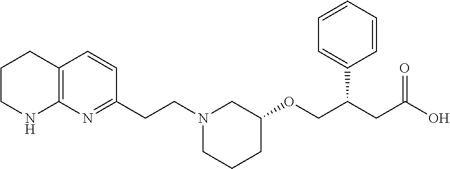 [SMILES: O=C(O)C[C@H](CO[C@@H]1CCCN(CCc2ccc3c(n2)NCCC3)C1)c1ccccc1] |
| 354 | 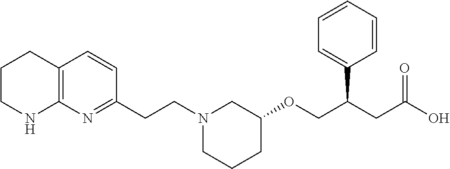 [SMILES: O=C(O)C[C@@H](CO[C@@H]1CCCN(CCc2ccc3c(n2)NCCC3)C1)c1ccccc1] |
| 355 | 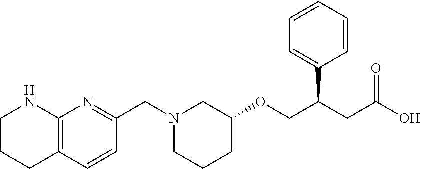 [SMILES: O=C(O)C[C@@H](CO[C@@H]1CCCN(Cc2ccc3c(n2)NCCC3)C1)c1ccccc1] |
| 356 | 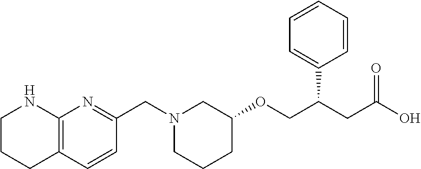 [SMILES: O=C(O)C[C@H](CO[C@@H]1CCCN(Cc2ccc3c(n2)NCCC3)C1)c1ccccc1] |
| 357 | 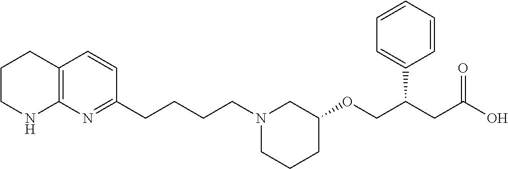 [SMILES: O=C(O)C[C@H](CO[C@@H]1CCCN(CCCCc2ccc3c(n2)NCCC3)C1)c1ccccc1] |
| 358 | 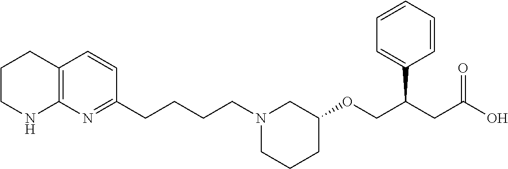 [SMILES: O=C(O)C[C@@H](CO[C@@H]1CCCN(CCCCc2ccc3c(n2)NCCC3)C1)c1ccccc1] |
| 359 | 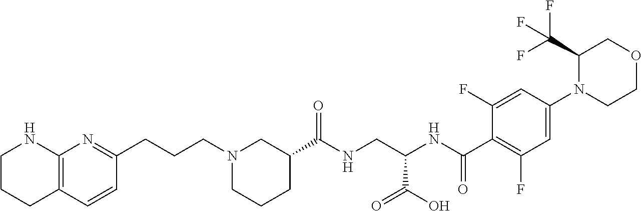 [SMILES: O=C(N[C@@H](CNC(=O)[C@@H]1CCCN(CCCc2ccc3c(n2)NCCC3)C1)C(=O)O)c1c(F)cc(N2CCOC[C@@H]2C(F)(F)F)cc1F] |
| 360 | 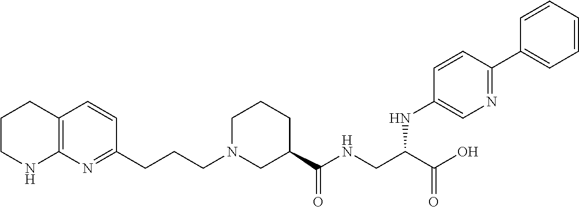 [SMILES: O=C(NC[C@H](Nc1ccc(-c2ccccc2)nc1)C(=O)O)[C@@H]1CCCN(CCCc2ccc3c(n2)NCCC3)C1] |
| 361 | 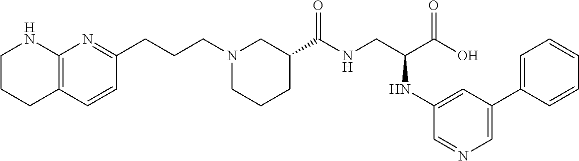 [SMILES: O=C(NC[C@H](Nc1cncc(-c2ccccc2)c1)C(=O)O)[C@@H]1CCCN(CCCc2ccc3c(n2)NCCC3)C1] |
| 362 | 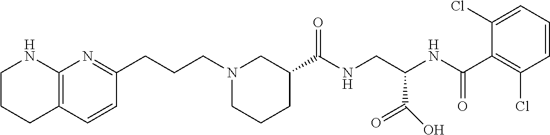 [SMILES: O=C(N[C@@H](CNC(=O)[C@@H]1CCCN(CCCc2ccc3c(n2)NCCC3)C1)C(=O)O)c1c(Cl)cccc1Cl] |
| 363 | 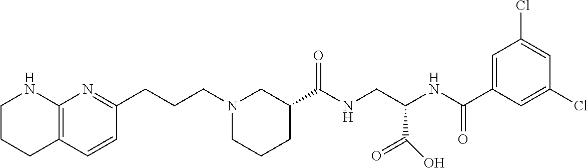 [SMILES: O=C(N[C@@H](CNC(=O)[C@@H]1CCCN(CCCc2ccc3c(n2)NCCC3)C1)C(=O)O)c1cc(Cl)cc(Cl)c1] |
| 364 | 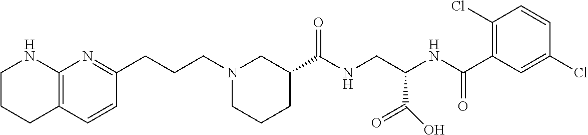 [SMILES: O=C(N[C@@H](CNC(=O)[C@@H]1CCCN(CCCc2ccc3c(n2)NCCC3)C1)C(=O)O)c1cc(Cl)ccc1Cl] |
| 365 | 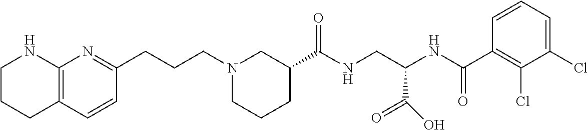 [SMILES: O=C(N[C@@H](CNC(=O)[C@@H]1CCCN(CCCc2ccc3c(n2)NCCC3)C1)C(=O)O)c1cccc(Cl)c1Cl] |
| 366 | 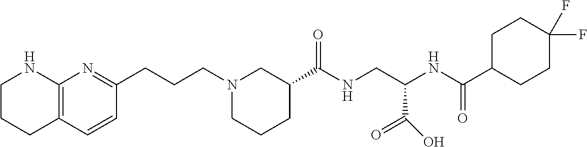 [SMILES: O=C(N[C@@H](CNC(=O)[C@@H]1CCCN(CCCc2ccc3c(n2)NCCC3)C1)C(=O)O)C1CCC(F)(F)CC1] |
| 367 | 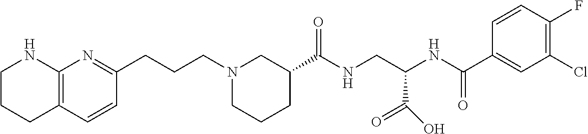 [SMILES: O=C(N[C@@H](CNC(=O)[C@@H]1CCCN(CCCc2ccc3c(n2)NCCC3)C1)C(=O)O)c1ccc(F)c(Cl)c1] |
| 368 | 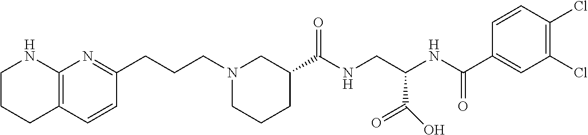 [SMILES: O=C(N[C@@H](CNC(=O)[C@@H]1CCCN(CCCc2ccc3c(n2)NCCC3)C1)C(=O)O)c1ccc(Cl)c(Cl)c1] |
| 369 | 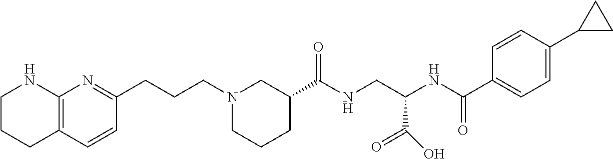 [SMILES: O=C(N[C@@H](CNC(=O)[C@@H]1CCCN(CCCc2ccc3c(n2)NCCC3)C1)C(=O)O)c1ccc(C2CC2)cc1] |
| 370 | 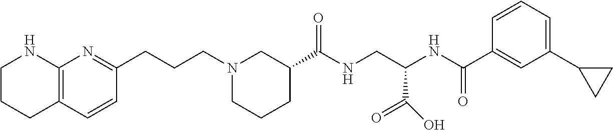 [SMILES: O=C(N[C@@H](CNC(=O)[C@@H]1CCCN(CCCc2ccc3c(n2)NCCC3)C1)C(=O)O)c1cccc(C2CC2)c1] |
| 371 | 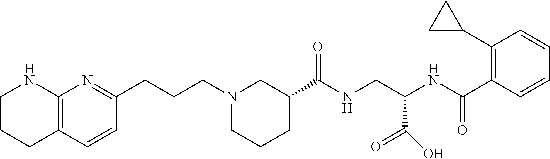 [SMILES: O=C(N[C@@H](CNC(=O)[C@@H]1CCCN(CCCc2ccc3c(n2)NCCC3)C1)C(=O)O)c1ccccc1C1CC1] |
| 372 | 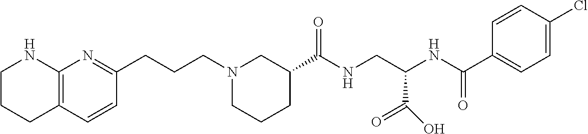 [SMILES: O=C(N[C@@H](CNC(=O)[C@@H]1CCCN(CCCc2ccc3c(n2)NCCC3)C1)C(=O)O)c1ccc(Cl)cc1] |
| 373 | 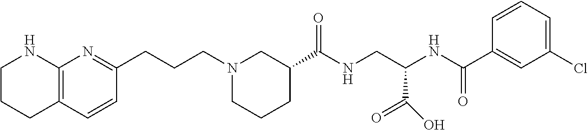 [SMILES: O=C(N[C@@H](CNC(=O)[C@@H]1CCCN(CCCc2ccc3c(n2)NCCC3)C1)C(=O)O)c1cccc(Cl)c1] |
| 374 | 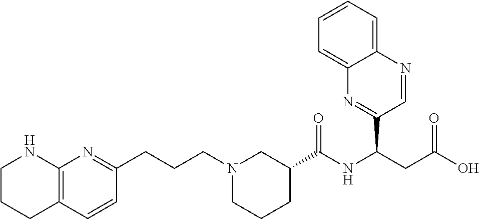 [SMILES: O=C(O)C[C@@H](NC(=O)[C@@H]1CCCN(CCCc2ccc3c(n2)NCCC3)C1)c1cnc2ccccc2n1] |
| 375 | 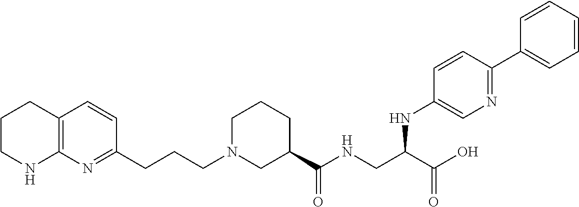 [SMILES: O=C(NC[C@@H](Nc1ccc(-c2ccccc2)nc1)C(=O)O)[C@@H]1CCCN(CCCc2ccc3c(n2)NCCC3)C1] |
| 376 | 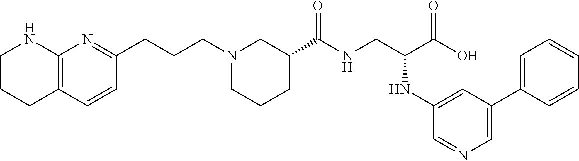 [SMILES: O=C(NC[C@@H](Nc1cncc(-c2ccccc2)c1)C(=O)O)[C@@H]1CCCN(CCCc2ccc3c(n2)NCCC3)C1] |
| 377 | 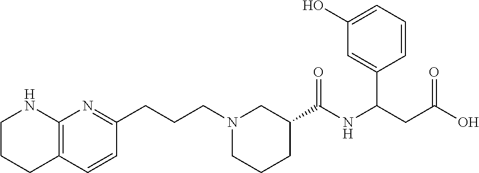 [SMILES: O=C(O)CC(NC(=O)[C@@H]1CCCN(CCCc2ccc3c(n2)NCCC3)C1)c1cccc(O)c1] |
| 378 | 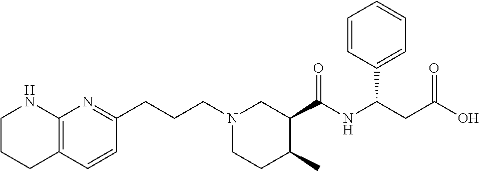 [SMILES: C[C@H]1CCN(CCCc2ccc3c(n2)NCCC3)C[C@H]1C(=O)N[C@@H](CC(=O)O)c1ccccc1] |
| 379 | 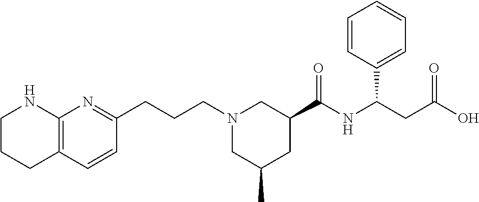 [SMILES: C[C@@H]1C[C@H](C(=O)N[C@@H](CC(=O)O)c2ccccc2)CN(CCCc2ccc3c(n2)NCCC3)C1] |
| 380 | 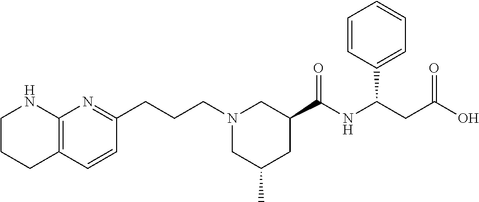 [SMILES: C[C@H]1C[C@H](C(=O)N[C@@H](CC(=O)O)c2ccccc2)CN(CCCc2ccc3c(n2)NCCC3)C1] |
| 381 | 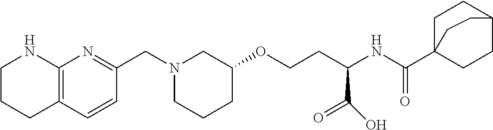 [SMILES: O=C(O)[C@@H](CCO[C@@H]1CCCN(Cc2ccc3c(n2)NCCC3)C1)NC(=O)C12CCC(CC1)CC2] |
| 382 | 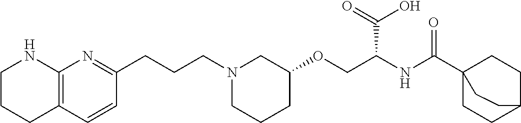 [SMILES: O=C(O)[C@@H](CO[C@@H]1CCCN(CCCc2ccc3c(n2)NCCC3)C1)NC(=O)C12CCC(CC1)CC2] |
| 383 | 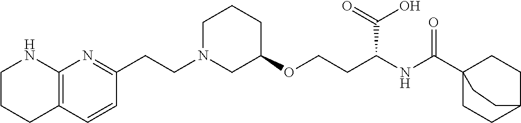 [SMILES: O=C(O)[C@@H](CCO[C@@H]1CCCN(CCc2ccc3c(n2)NCCC3)C1)NC(=O)C12CCC(CC1)CC2] |
| 384 | 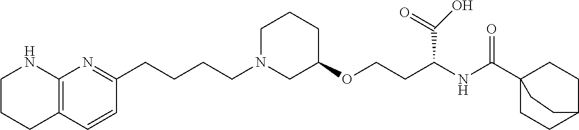 [SMILES: O=C(O)[C@@H](CCO[C@@H]1CCCN(CCCCc2ccc3c(n2)NCCC3)C1)NC(=O)C12CCC(CC1)CC2] |
| 385 | 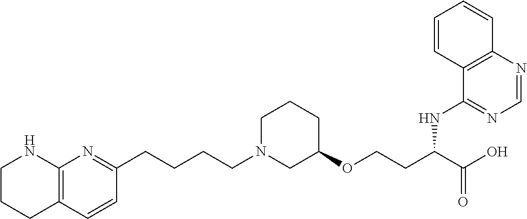 [SMILES: O=C(O)[C@H](CCO[C@@H]1CCCN(CCCCc2ccc3c(n2)NCCC3)C1)Nc1ncnc2ccccc12] |
| 386 | 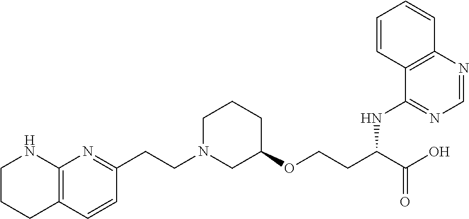 [SMILES: O=C(O)[C@H](CCO[C@@H]1CCCN(CCc2ccc3c(n2)NCCC3)C1)Nc1ncnc2ccccc12] |
| 387 | 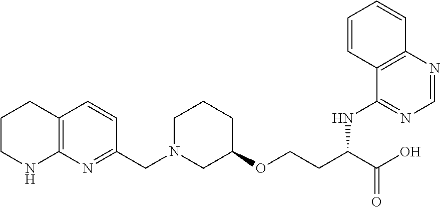 [SMILES: O=C(O)[C@H](CCO[C@@H]1CCCN(Cc2ccc3c(n2)NCCC3)C1)Nc1ncnc2ccccc12] |
| 388 | 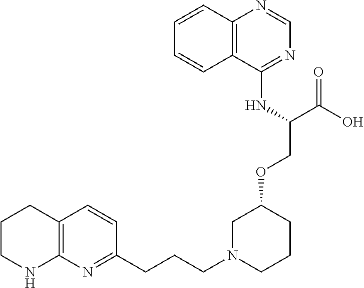 [SMILES: O=C(O)[C@H](CO[C@@H]1CCCN(CCCc2ccc3c(n2)NCCC3)C1)Nc1ncnc2ccccc12] |
| 389 | 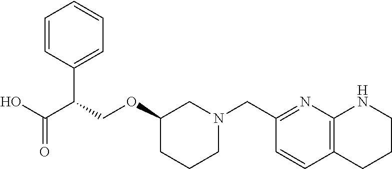 [SMILES: O=C(O)[C@@H](CO[C@@H]1CCCN(Cc2ccc3c(n2)NCCC3)C1)c1ccccc1] |
| 390 | 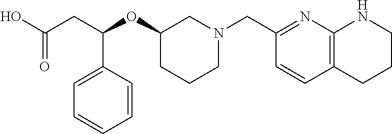 [SMILES: O=C(O)C[C@@H](O[C@@H]1CCCN(Cc2ccc3c(n2)NCCC3)C1)c1ccccc1] |
| 391 | 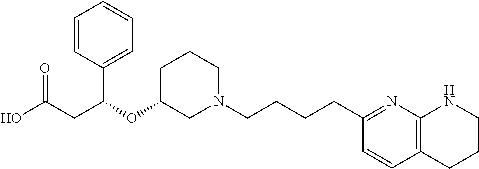 [SMILES: O=C(O)C[C@@H](O[C@@H]1CCCN(CCCCc2ccc3c(n2)NCCC3)C1)c1ccccc1] |
| 392 | 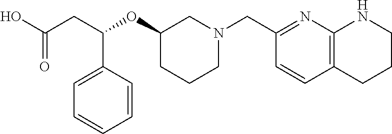 [SMILES: O=C(O)C[C@H](O[C@@H]1CCCN(Cc2ccc3c(n2)NCCC3)C1)c1ccccc1] |
| 393 | 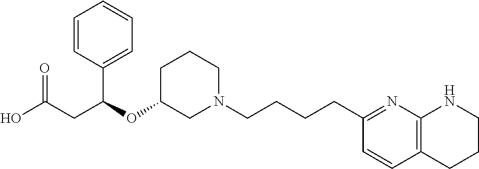 [SMILES: O=C(O)C[C@H](O[C@@H]1CCCN(CCCCc2ccc3c(n2)NCCC3)C1)c1ccccc1] |
| 394 | 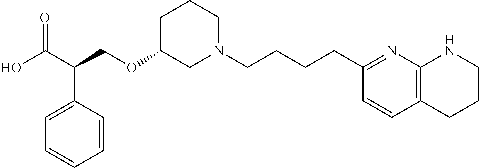 [SMILES: O=C(O)[C@@H](CO[C@@H]1CCCN(CCCCc2ccc3c(n2)NCCC3)C1)c1ccccc1] |
| 395 | 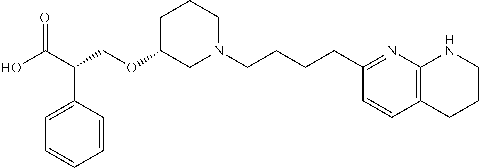 [SMILES: O=C(O)[C@H](CO[C@@H]1CCCN(CCCCc2ccc3c(n2)NCCC3)C1)c1ccccc1] |
| 396 | 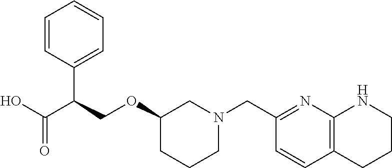 [SMILES: O=C(O)[C@H](CO[C@@H]1CCCN(Cc2ccc3c(n2)NCCC3)C1)c1ccccc1] |
| 397 | 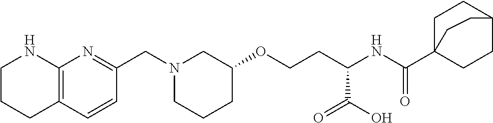 [SMILES: O=C(O)[C@H](CCO[C@@H]1CCCN(Cc2ccc3c(n2)NCCC3)C1)NC(=O)C12CCC(CC1)CC2] |
| 398 | 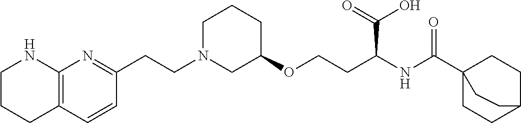 [SMILES: O=C(O)[C@H](CCO[C@@H]1CCCN(CCc2ccc3c(n2)NCCC3)C1)NC(=O)C12CCC(CC1)CC2] |
| 399 | 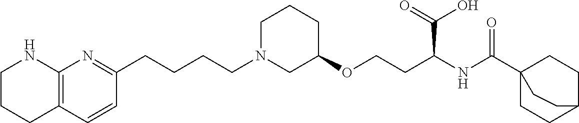 [SMILES: O=C(O)[C@H](CCO[C@@H]1CCCN(CCCCc2ccc3c(n2)NCCC3)C1)NC(=O)C12CCC(CC1)CC2] |
| 400 | 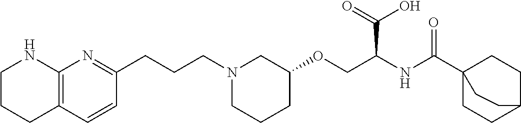 [SMILES: O=C(O)[C@H](CO[C@@H]1CCCN(CCCc2ccc3c(n2)NCCC3)C1)NC(=O)C12CCC(CC1)CC2] |
| 401 | 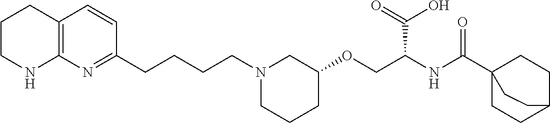 [SMILES: O=C(O)[C@@H](CO[C@@H]1CCCN(CCCCc2ccc3c(n2)NCCC3)C1)NC(=O)C12CCC(CC1)CC2] |
| 402 | 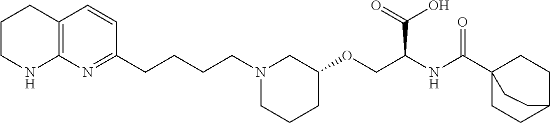 [SMILES: O=C(O)[C@H](CO[C@@H]1CCCN(CCCCc2ccc3c(n2)NCCC3)C1)NC(=O)C12CCC(CC1)CC2] |
| 403 | 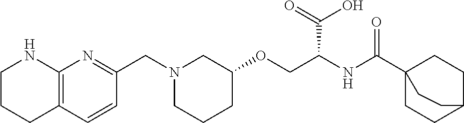 [SMILES: O=C(O)[C@@H](CO[C@@H]1CCCN(Cc2ccc3c(n2)NCCC3)C1)NC(=O)C12CCC(CC1)CC2] |
| 404 | 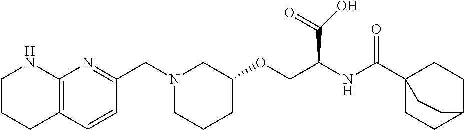 [SMILES: O=C(O)[C@H](CO[C@@H]1CCCN(Cc2ccc3c(n2)NCCC3)C1)NC(=O)C12CCC(CC1)CC2] |
| 405 | 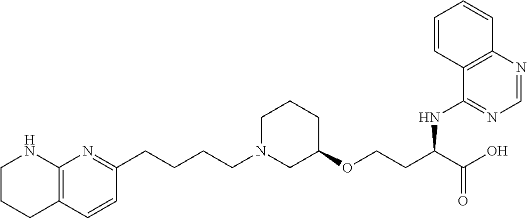 [SMILES: O=C(O)[C@@H](CCO[C@@H]1CCCN(CCCCc2ccc3c(n2)NCCC3)C1)Nc1ncnc2ccccc12] |
| 406 | 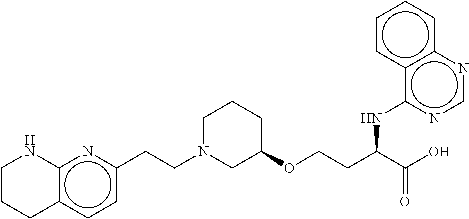 |
| 407 | 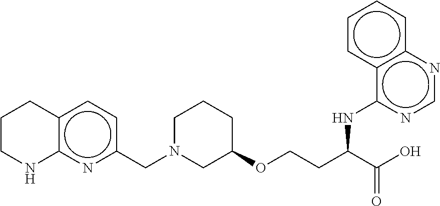 |
| 408 | 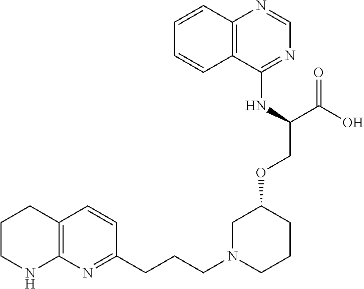 [SMILES: O=C(O)[C@@H](CO[C@@H]1CCCN(CCCc2ccc3c(n2)NCCC3)C1)Nc1ncnc2ccccc12] |
| 409 | 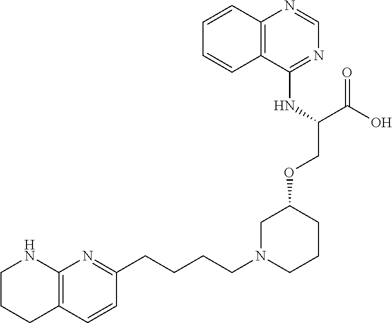 [SMILES: O=C(O)[C@H](CO[C@@H]1CCCN(CCCCc2ccc3c(n2)NCCC3)C1)Nc1ncnc2ccccc12] |
| 410 | 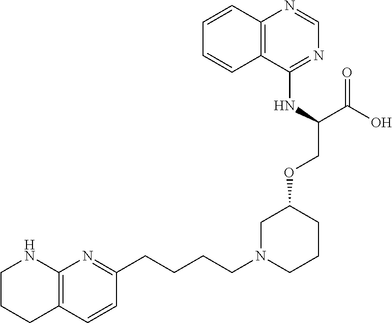 [SMILES: O=C(O)[C@@H](CO[C@@H]1CCCN(CCCCc2ccc3c(n2)NCCC3)C1)Nc1ncnc2ccccc12] |
| 411 | 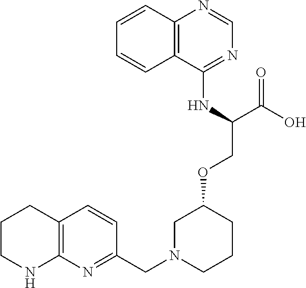 [SMILES: O=C(O)[C@@H](CO[C@@H]1CCCN(Cc2ccc3c(n2)NCCC3)C1)Nc1ncnc2ccccc12] |
| 412 | 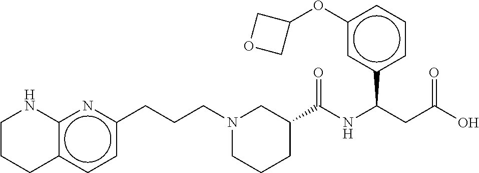 |
| 413 | 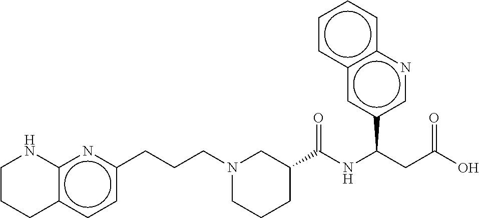 |
| 414 | 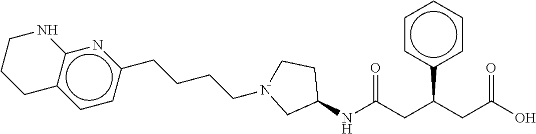 |
| 415 | 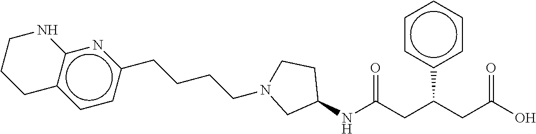 |
| 416 | 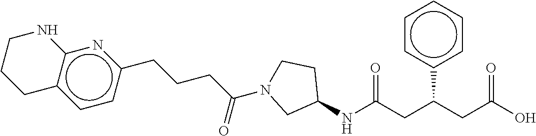 |
| 417 | 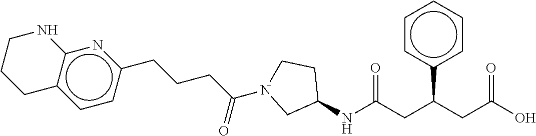 |
| 418 | 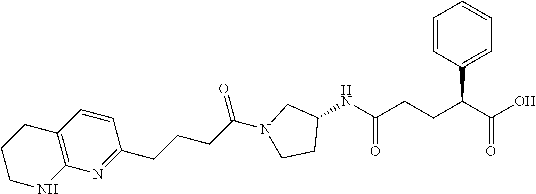 [SMILES: O=C(CC[C@H](C(=O)O)c1ccccc1)N[C@@H]1CCN(C(=O)CCCc2ccc3c(n2)NCCC3)C1] |
| 419 | 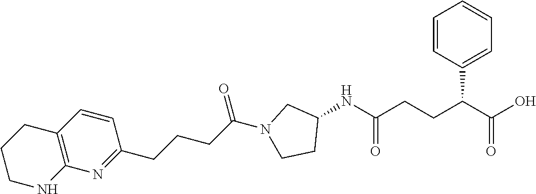 [SMILES: O=C(CC[C@@H](C(=O)O)c1ccccc1)N[C@@H]1CCN(C(=O)CCCc2ccc3c(n2)NCCC3)C1] |
| 420 | 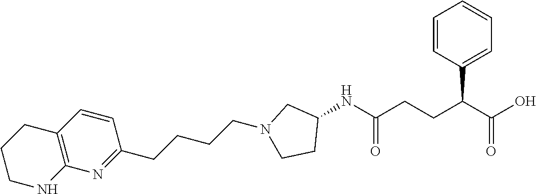 [SMILES: O=C(CC[C@H](C(=O)O)c1ccccc1)N[C@@H]1CCN(CCCCc2ccc3c(n2)NCCC3)C1] |
| 421 | 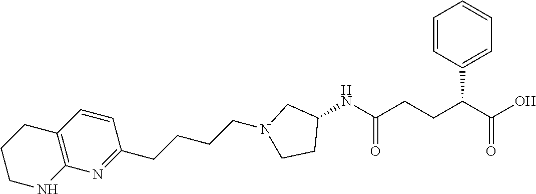 [SMILES: O=C(CC[C@@H](C(=O)O)c1ccccc1)N[C@@H]1CCN(CCCCc2ccc3c(n2)NCCC3)C1] |
| 422 | 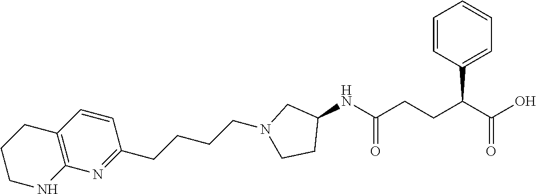 [SMILES: O=C(CC[C@H](C(=O)O)c1ccccc1)N[C@H]1CCN(CCCCc2ccc3c(n2)NCCC3)C1] |
| 423 | 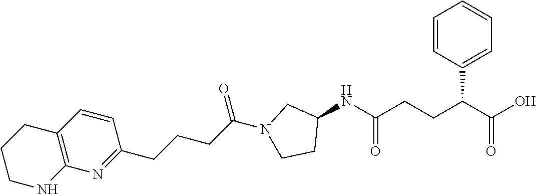 [SMILES: O=C(CC[C@@H](C(=O)O)c1ccccc1)N[C@H]1CCN(C(=O)CCCc2ccc3c(n2)NCCC3)C1] |
| 424 | 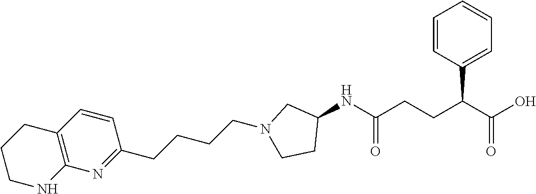 [SMILES: O=C(CC[C@H](C(=O)O)c1ccccc1)N[C@H]1CCN(CCCCc2ccc3c(n2)NCCC3)C1] |
| 425 | 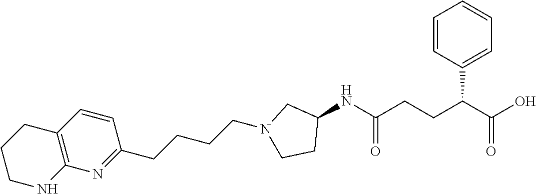 [SMILES: O=C(CC[C@@H](C(=O)O)c1ccccc1)N[C@H]1CCN(CCCCc2ccc3c(n2)NCCC3)C1] |
| 426 | 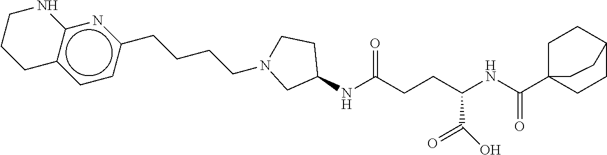 |
| 427 | 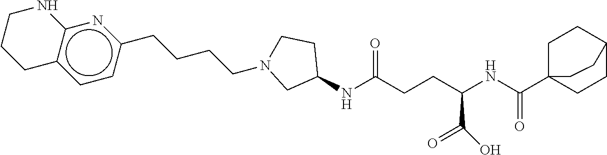 |
| 428 | 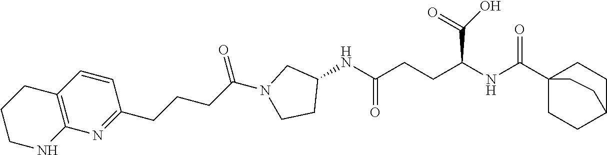 [SMILES: O=C(CC[C@H](NC(=O)C12CCC(CC1)CC2)C(=O)O)N[C@@H]1CCN(C(=O)CCCc2ccc3c(n2)NCCC3)C1] |
| 429 | 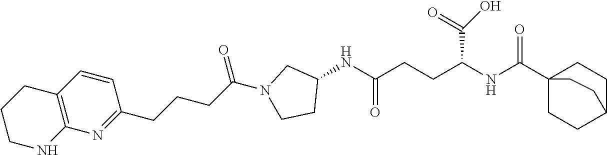 [SMILES: O=C(CC[C@@H](NC(=O)C12CCC(CC1)CC2)C(=O)O)N[C@@H]1CCN(C(=O)CCCc2ccc3c(n2)NCCC3)C1] |
| 430 | 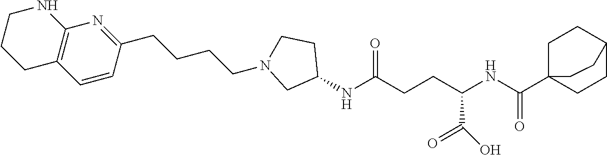 [SMILES: O=C(CC[C@H](NC(=O)C12CCC(CC1)CC2)C(=O)O)N[C@H]1CCN(CCCCc2ccc3c(n2)NCCC3)C1] |
| 431 | 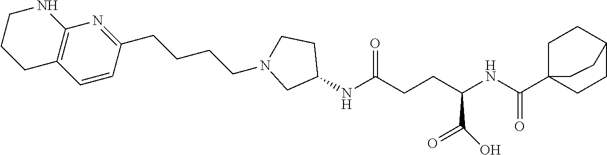 [SMILES: O=C(CC[C@@H](NC(=O)C12CCC(CC1)CC2)C(=O)O)N[C@H]1CCN(CCCCc2ccc3c(n2)NCCC3)C1] |
| 432 | 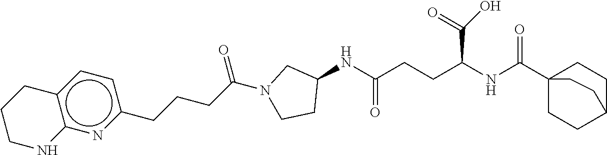 |
| 433 | 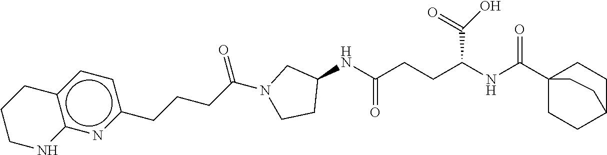 |
| 434 | 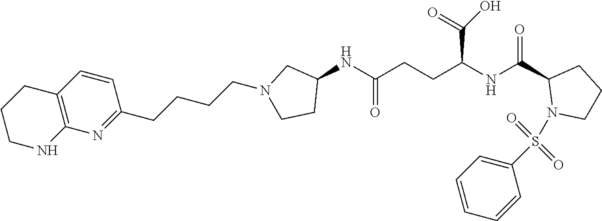 [SMILES: O=C(CC[C@H](NC(=O)[C@H]1CCCN1S(=O)(=O)c1ccccc1)C(=O)O)N[C@H]1CCN(CCCCc2ccc3c(n2)NCCC3)C1] |
| 435 | 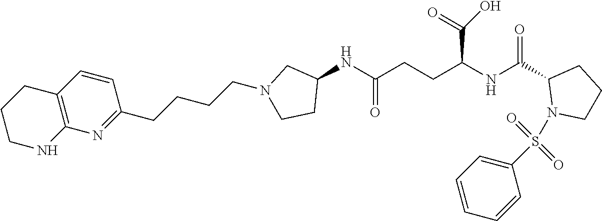 [SMILES: O=C(CC[C@H](NC(=O)[C@@H]1CCCN1S(=O)(=O)c1ccccc1)C(=O)O)N[C@H]1CCN(CCCCc2ccc3c(n2)NCCC3)C1] |
| 436 | 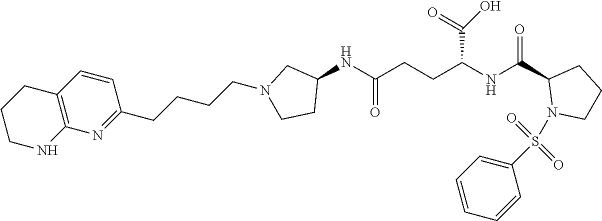 [SMILES: O=C(CC[C@@H](NC(=O)[C@H]1CCCN1S(=O)(=O)c1ccccc1)C(=O)O)N[C@H]1CCN(CCCCc2ccc3c(n2)NCCC3)C1] |
| 437 | 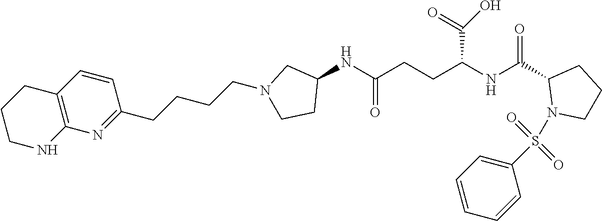 [SMILES: O=C(CC[C@@H](NC(=O)[C@@H]1CCCN1S(=O)(=O)c1ccccc1)C(=O)O)N[C@H]1CCN(CCCCc2ccc3c(n2)NCCC3)C1] |
| 438 | 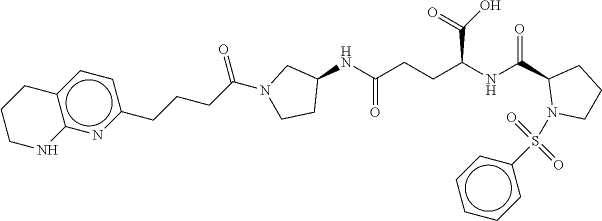 |
| 439 | 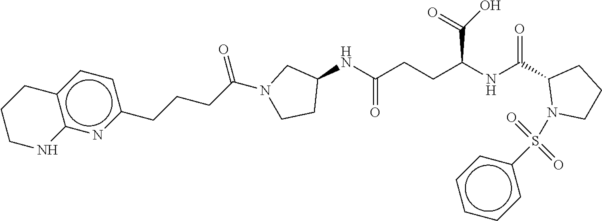 |
| 440 | 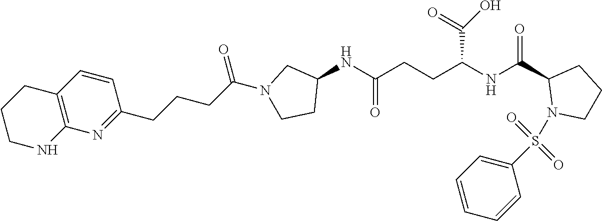 [SMILES: O=C(CC[C@@H](NC(=O)[C@H]1CCCN1S(=O)(=O)c1ccccc1)C(=O)O)N[C@H]1CCN(C(=O)CCCc2ccc3c(n2)NCCC3)C1] |
| 441 | 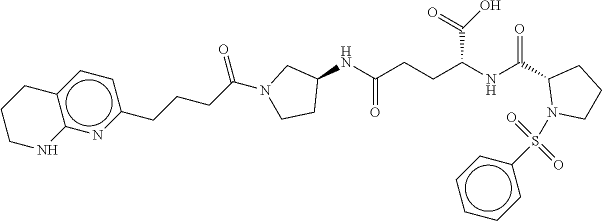 |
| 442 | 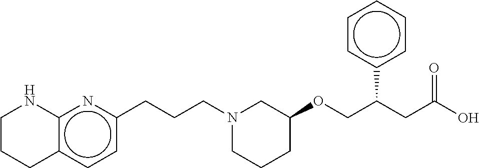 |
| 443 | 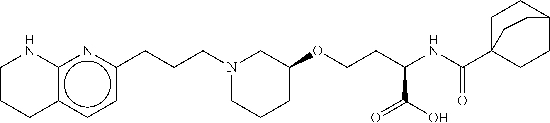 |
| 444 | 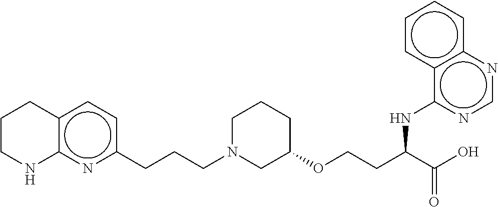 |
| 445 | 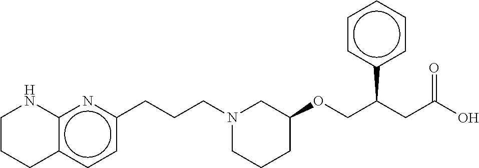 |
| 446 | 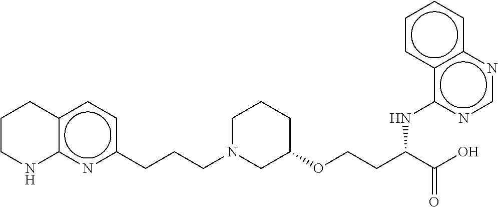 |
| 447 | 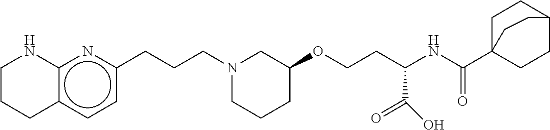 |
| 448 | 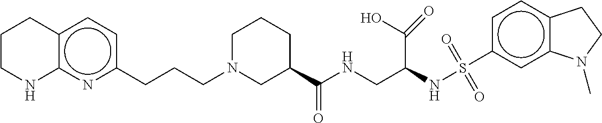 |
| 449 | 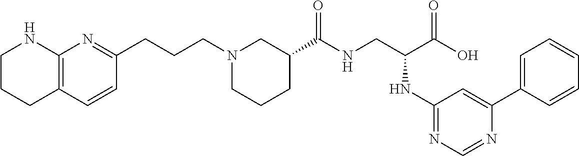 [SMILES: O=C(NC[C@@H](Nc1cc(-c2ccccc2)ncn1)C(=O)O)[C@@H]1CCCN(CCCc2ccc3c(n2)NCCC3)C1] |
| 450 | 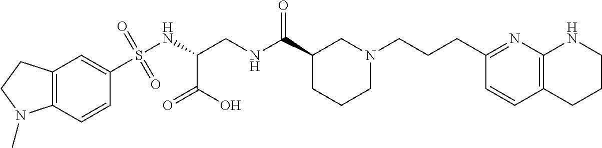 [SMILES: CN1CCc2cc(S(=O)(=O)N[C@H](CNC(=O)[C@@H]3CCCN(CCCc4ccc5c(n4)NCCC5)C3)C(=O)O)ccc21] |
| 451 | 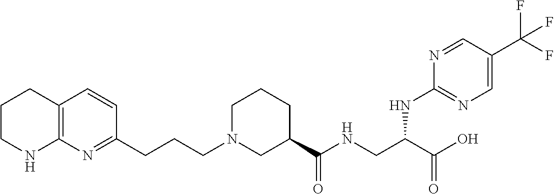 [SMILES: O=C(NC[C@H](Nc1ncc(C(F)(F)F)cn1)C(=O)O)[C@@H]1CCCN(CCCc2ccc3c(n2)NCCC3)C1] |
| 452 | 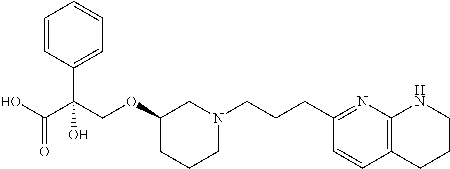 [SMILES: O=C(O)[C@](O)(CO[C@@H]1CCCN(CCCc2ccc3c(n2)NCCC3)C1)c1ccccc1] |
| 453 | 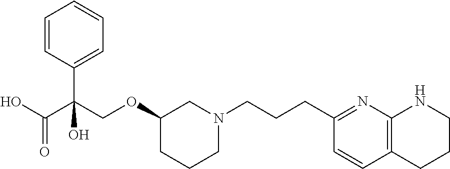 [SMILES: O=C(O)[C@@](O)(CO[C@@H]1CCCN(CCCc2ccc3c(n2)NCCC3)C1)c1ccccc1] |
| 454 | 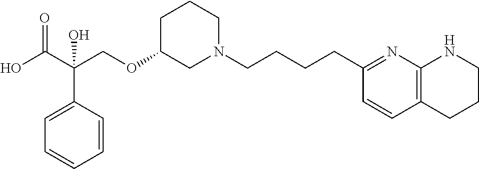 [SMILES: O=C(O)[C@@](O)(CO[C@@H]1CCCN(CCCCc2ccc3c(n2)NCCC3)C1)c1ccccc1] |
| 455 | 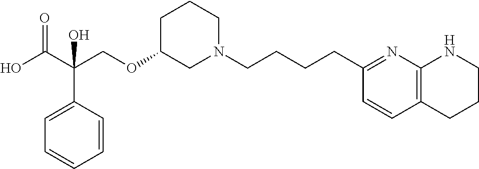 [SMILES: O=C(O)[C@](O)(CO[C@@H]1CCCN(CCCCc2ccc3c(n2)NCCC3)C1)c1ccccc1] |
| 456 | 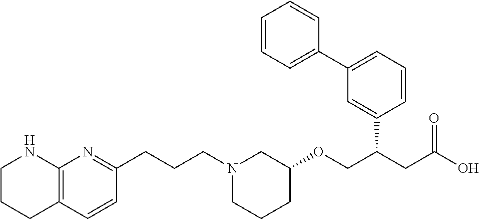 [SMILES: O=C(O)C[C@H](CO[C@@H]1CCCN(CCCc2ccc3c(n2)NCCC3)C1)c1cccc(-c2ccccc2)c1] |
| 457 | 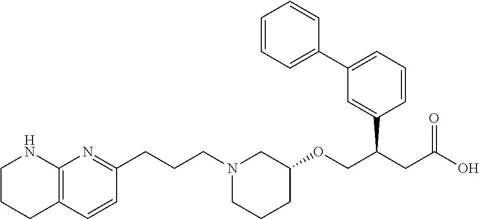 [SMILES: O=C(O)C[C@@H](CO[C@@H]1CCCN(CCCc2ccc3c(n2)NCCC3)C1)c1cccc(-c2ccccc2)c1] |
| 458 | 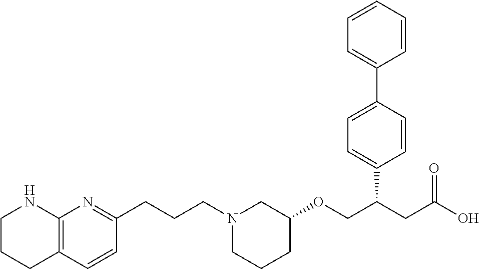 [SMILES: O=C(O)C[C@H](CO[C@@H]1CCCN(CCCc2ccc3c(n2)NCCC3)C1)c1ccc(-c2ccccc2)cc1] |
| 459 | 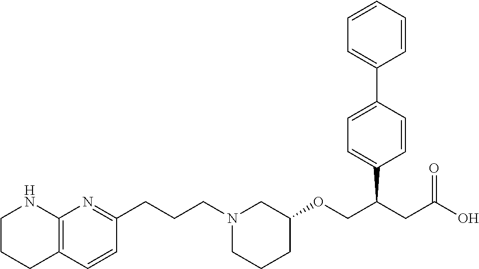 [SMILES: O=C(O)C[C@@H](CO[C@@H]1CCCN(CCCc2ccc3c(n2)NCCC3)C1)c1ccc(-c2ccccc2)cc1] |
| 460 | 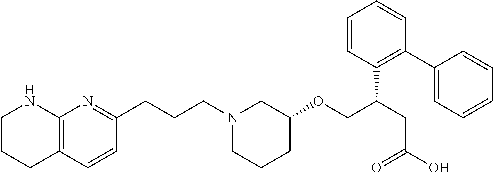 [SMILES: O=C(O)C[C@H](CO[C@@H]1CCCN(CCCc2ccc3c(n2)NCCC3)C1)c1ccccc1-c1ccccc1] |
| 461 | 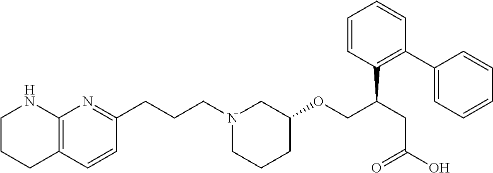 [SMILES: O=C(O)C[C@@H](CO[C@@H]1CCCN(CCCc2ccc3c(n2)NCCC3)C1)c1ccccc1-c1ccccc1] |
| 462 | 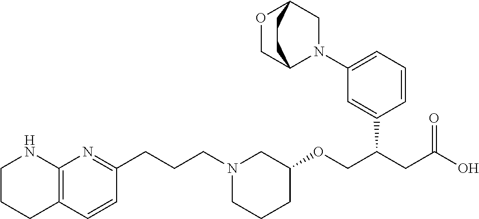 [SMILES: O=C(O)C[C@H](CO[C@@H]1CCCN(CCCc2ccc3c(n2)NCCC3)C1)c1cccc(N2C[C@@H]3CC[C@H]2CO3)c1] |
| 463 | 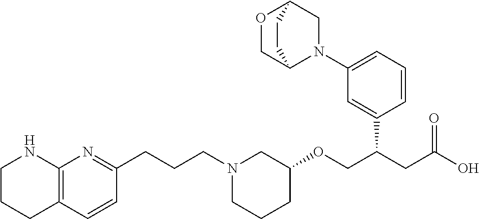 [SMILES: O=C(O)C[C@H](CO[C@@H]1CCCN(CCCc2ccc3c(n2)NCCC3)C1)c1cccc(N2C[C@H]3CC[C@@H]2CO3)c1] |
| 464 | 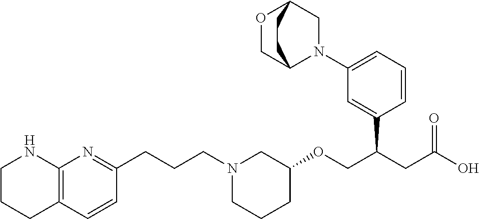 [SMILES: O=C(O)C[C@@H](CO[C@@H]1CCCN(CCCc2ccc3c(n2)NCCC3)C1)c1cccc(N2C[C@@H]3CC[C@H]2CO3)c1] |
| 465 | 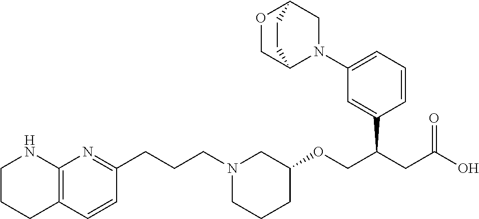 [SMILES: O=C(O)C[C@@H](CO[C@@H]1CCCN(CCCc2ccc3c(n2)NCCC3)C1)c1cccc(N2C[C@H]3CC[C@@H]2CO3)c1] |
| 466 | 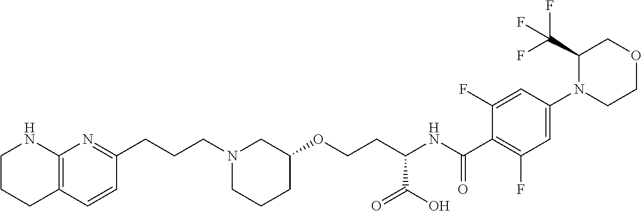 [SMILES: O=C(N[C@@H](CCO[C@@H]1CCCN(CCCc2ccc3c(n2)NCCC3)C1)C(=O)O)c1c(F)cc(N2CCOC[C@@H]2C(F)(F)F)cc1F] |
| 467 | 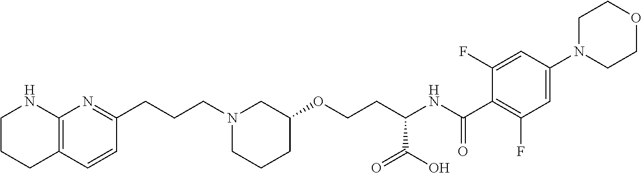 [SMILES: O=C(N[C@@H](CCO[C@@H]1CCCN(CCCc2ccc3c(n2)NCCC3)C1)C(=O)O)c1c(F)cc(N2CCOCC2)cc1F] |
| 468 | 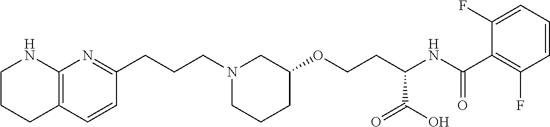 [SMILES: O=C(N[C@@H](CCO[C@@H]1CCCN(CCCc2ccc3c(n2)NCCC3)C1)C(=O)O)c1c(F)cccc1F] |
| 469 | 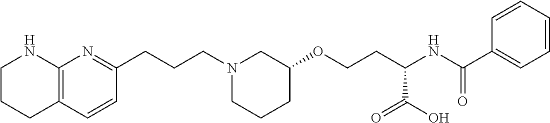 [SMILES: O=C(N[C@@H](CCO[C@@H]1CCCN(CCCc2ccc3c(n2)NCCC3)C1)C(=O)O)c1ccccc1] |
| 470 | 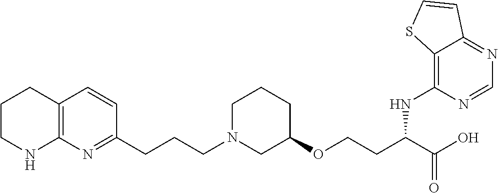 [SMILES: O=C(O)[C@H](CCO[C@@H]1CCCN(CCCc2ccc3c(n2)NCCC3)C1)Nc1ncnc2ccsc12] |
| 471 | 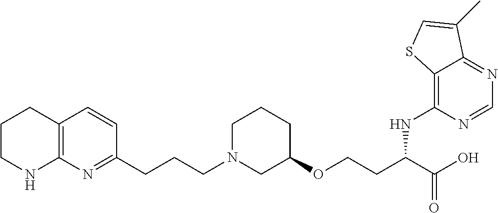 [SMILES: Cc1csc2c(N[C@@H](CCO[C@@H]3CCCN(CCCc4ccc5c(n4)NCCC5)C3)C(=O)O)ncnc12] |
| 472 | 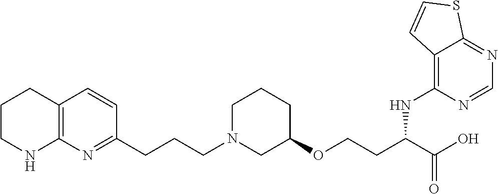 [SMILES: O=C(O)[C@H](CCO[C@@H]1CCCN(CCCc2ccc3c(n2)NCCC3)C1)Nc1ncnc2sccc12] |
| 473 | 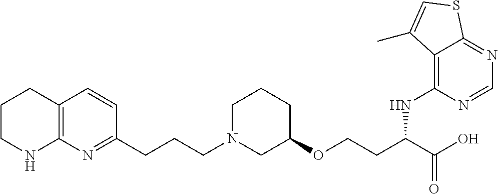 [SMILES: Cc1csc2ncnc(N[C@@H](CCO[C@@H]3CCCN(CCCc4ccc5c(n4)NCCC5)C3)C(=O)O)c12] |
| 474 | 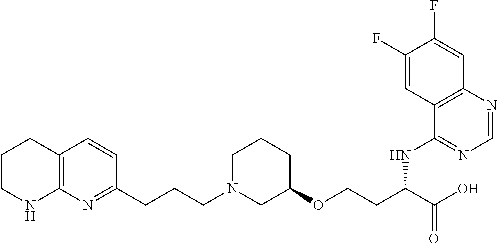 [SMILES: O=C(O)[C@H](CCO[C@@H]1CCCN(CCCc2ccc3c(n2)NCCC3)C1)Nc1ncnc2cc(F)c(F)cc12] |
| 475 | 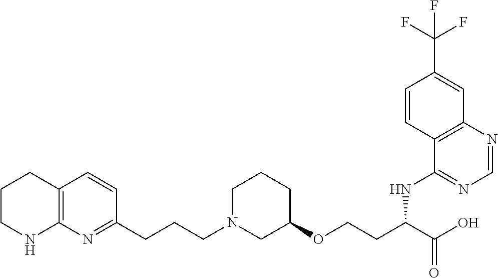 [SMILES: O=C(O)[C@H](CCO[C@@H]1CCCN(CCCc2ccc3c(n2)NCCC3)C1)Nc1ncnc2cc(C(F)(F)F)ccc12] |
| 476 | 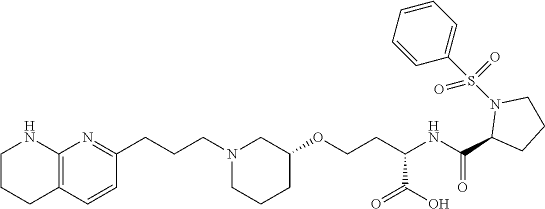 [SMILES: O=C(O)[C@H](CCO[C@@H]1CCCN(CCCc2ccc3c(n2)NCCC3)C1)NC(=O)[C@@H]1CCCN1S(=O)(=O)c1ccccc1] |
| 477 | 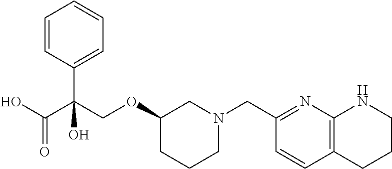 [SMILES: O=C(O)[C@@](O)(CO[C@@H]1CCCN(Cc2ccc3c(n2)NCCC3)C1)c1ccccc1] |
| 478 | 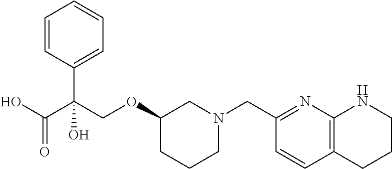 [SMILES: O=C(O)[C@](O)(CO[C@@H]1CCCN(Cc2ccc3c(n2)NCCC3)C1)c1ccccc1] |
| 479 | 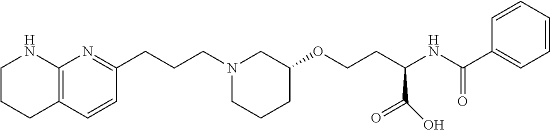 [SMILES: O=C(N[C@H](CCO[C@@H]1CCCN(CCCc2ccc3c(n2)NCCC3)C1)C(=O)O)c1ccccc1] |
| 480 | 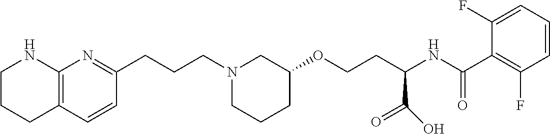 [SMILES: O=C(N[C@H](CCO[C@@H]1CCCN(CCCc2ccc3c(n2)NCCC3)C1)C(=O)O)c1c(F)cccc1F] |
| 481 | 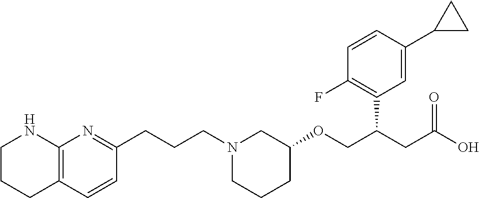 [SMILES: O=C(O)C[C@H](CO[C@@H]1CCCN(CCCc2ccc3c(n2)NCCC3)C1)c1cc(C2CC2)ccc1F] |
| 482 | 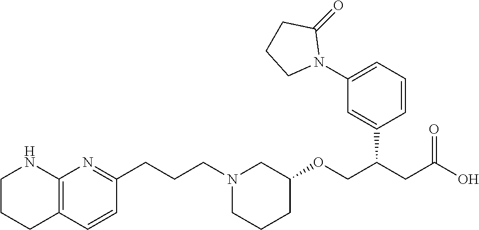 [SMILES: O=C(O)C[C@H](CO[C@@H]1CCCN(CCCc2ccc3c(n2)NCCC3)C1)c1cccc(N2CCCC2=O)c1] |
| 483 | 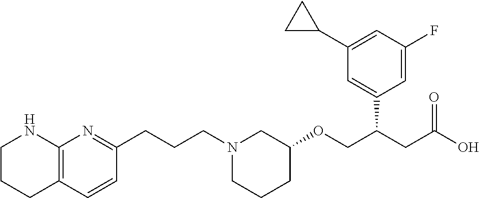 [SMILES: O=C(O)C[C@H](CO[C@@H]1CCCN(CCCc2ccc3c(n2)NCCC3)C1)c1cc(F)cc(C2CC2)c1] |
| 484 | 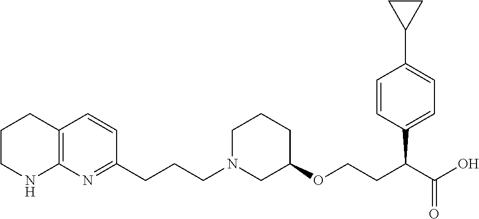 [SMILES: O=C(O)[C@@H](CCO[C@@H]1CCCN(CCCc2ccc3c(n2)NCCC3)C1)c1ccc(C2CC2)cc1] |
| 485 | 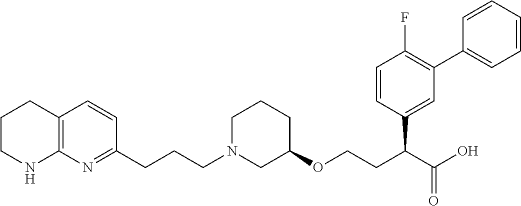 [SMILES: O=C(O)[C@@H](CCO[C@@H]1CCCN(CCCc2ccc3c(n2)NCCC3)C1)c1ccc(F)c(-c2ccccc2)c1] |
| 486 | 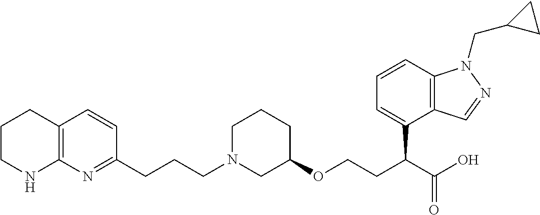 [SMILES: O=C(O)[C@@H](CCO[C@@H]1CCCN(CCCc2ccc3c(n2)NCCC3)C1)c1cccc2c1cnn2CC1CC1] |
| 487 | 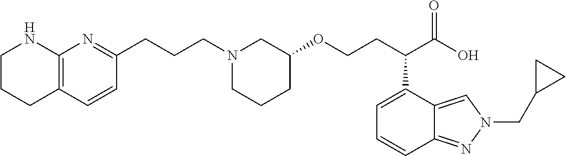 [SMILES: O=C(O)[C@@H](CCO[C@@H]1CCCN(CCCc2ccc3c(n2)NCCC3)C1)c1cccc2nn(CC3CC3)cc12] |
| 488 | 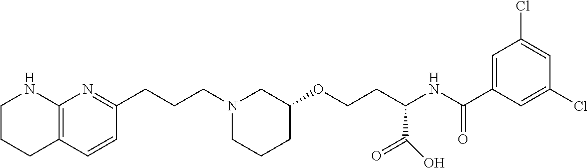 [SMILES: O=C(N[C@@H](CCO[C@@H]1CCCN(CCCc2ccc3c(n2)NCCC3)C1)C(=O)O)c1cc(Cl)cc(Cl)c1] |
| 489 | 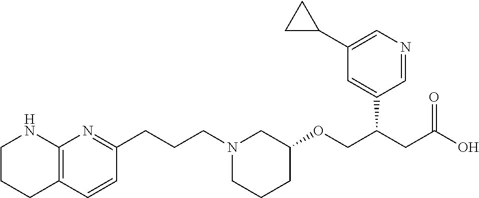 [SMILES: O=C(O)C[C@H](CO[C@@H]1CCCN(CCCc2ccc3c(n2)NCCC3)C1)c1cncc(C2CC2)c1] |
| 490 | 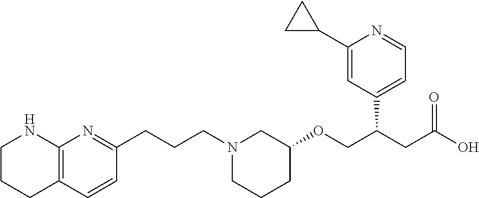 [SMILES: O=C(O)C[C@H](CO[C@@H]1CCCN(CCCc2ccc3c(n2)NCCC3)C1)c1ccnc(C2CC2)c1] |
| 491 | 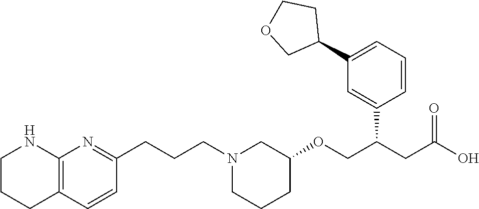 [SMILES: O=C(O)C[C@H](CO[C@@H]1CCCN(CCCc2ccc3c(n2)NCCC3)C1)c1cccc([C@@H]2CCOC2)c1] |
| 492 | 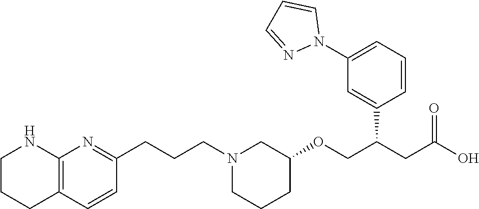 [SMILES: O=C(O)C[C@H](CO[C@@H]1CCCN(CCCc2ccc3c(n2)NCCC3)C1)c1cccc(-n2cccn2)c1] |
| 493 | 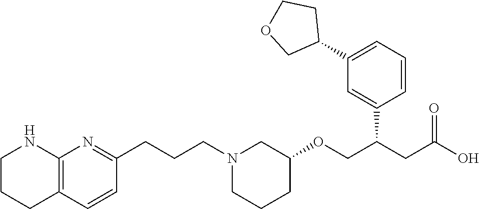 [SMILES: O=C(O)C[C@H](CO[C@@H]1CCCN(CCCc2ccc3c(n2)NCCC3)C1)c1cccc([C@H]2CCOC2)c1] |
| 494 | 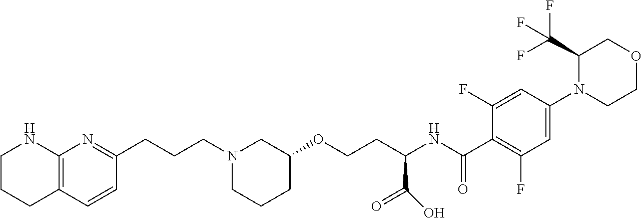 [SMILES: O=C(N[C@H](CCO[C@@H]1CCCN(CCCc2ccc3c(n2)NCCC3)C1)C(=O)O)c1c(F)cc(N2CCOC[C@@H]2C(F)(F)F)cc1F] |
| 495 | 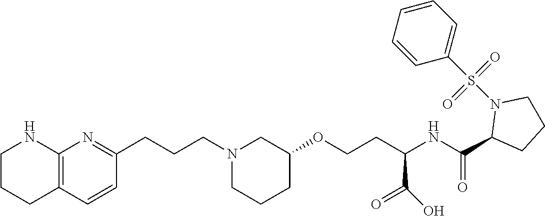 [SMILES: O=C(O)[C@@H](CCO[C@@H]1CCCN(CCCc2ccc3c(n2)NCCC3)C1)NC(=O)[C@@H]1CCCN1S(=O)(=O)c1ccccc1] |
| 496 | 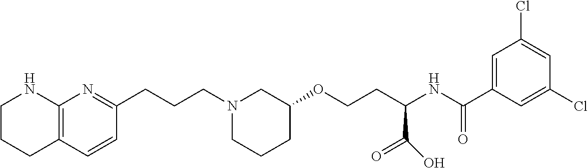 [SMILES: O=C(N[C@H](CCO[C@@H]1CCCN(CCCc2ccc3c(n2)NCCC3)C1)C(=O)O)c1cc(Cl)cc(Cl)c1] |
| 497 | 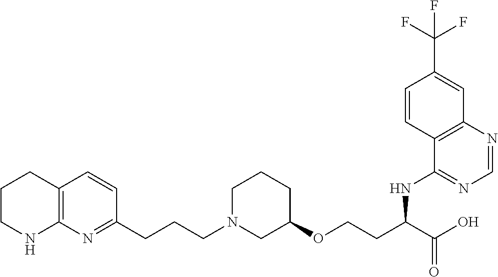 [SMILES: O=C(O)[C@@H](CCO[C@@H]1CCCN(CCCc2ccc3c(n2)NCCC3)C1)Nc1ncnc2cc(C(F)(F)F)ccc12] |
| 498 | 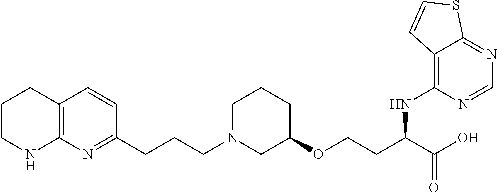 [SMILES: O=C(O)[C@@H](CCO[C@@H]1CCCN(CCCc2ccc3c(n2)NCCC3)C1)Nc1ncnc2sccc12] |
| 499 | 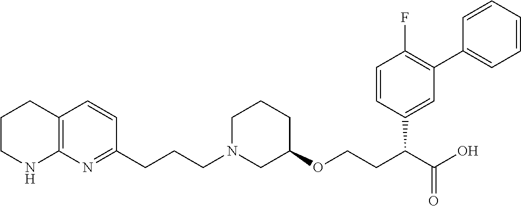 [SMILES: O=C(O)[C@H](CCO[C@@H]1CCCN(CCCc2ccc3c(n2)NCCC3)C1)c1ccc(F)c(-c2ccccc2)c1] |
| 500 | 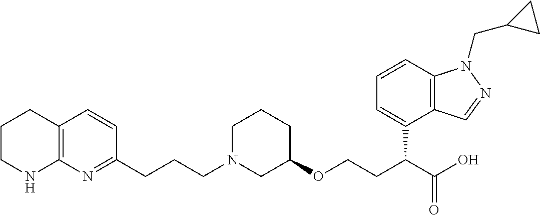 [SMILES: O=C(O)[C@H](CCO[C@@H]1CCCN(CCCc2ccc3c(n2)NCCC3)C1)c1cccc2c1cnn2CC1CC1] |
| 501 | 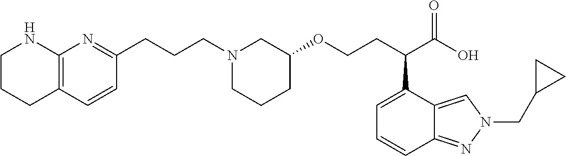 [SMILES: O=C(O)[C@H](CCO[C@@H]1CCCN(CCCc2ccc3c(n2)NCCC3)C1)c1cccc2nn(CC3CC3)cc12] |
| 502 | 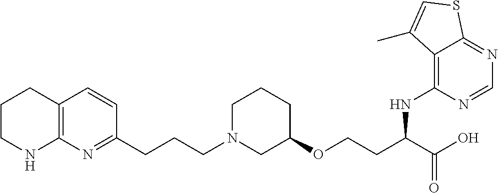 [SMILES: Cc1csc2ncnc(N[C@H](CCO[C@@H]3CCCN(CCCc4ccc5c(n4)NCCC5)C3)C(=O)O)c12] |
| 503 | 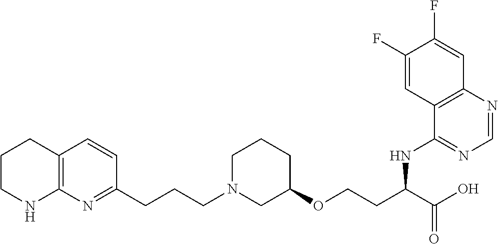 [SMILES: O=C(O)[C@@H](CCO[C@@H]1CCCN(CCCc2ccc3c(n2)NCCC3)C1)Nc1ncnc2cc(F)c(F)cc12] |
| 504 | 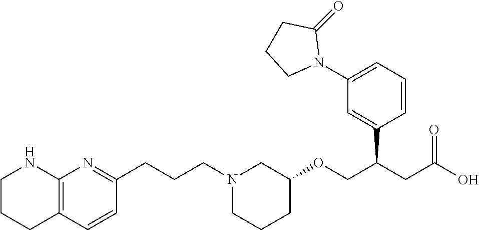 [SMILES: O=C(O)C[C@@H](CO[C@@H]1CCCN(CCCc2ccc3c(n2)NCCC3)C1)c1cccc(N2CCCC2=O)c1] |
| 505 | 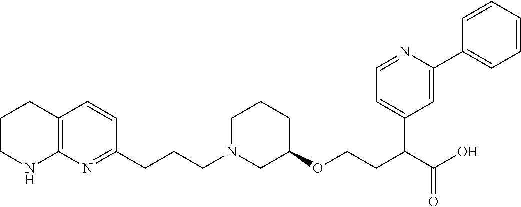 [SMILES: O=C(O)C(CCO[C@@H]1CCCN(CCCc2ccc3c(n2)NCCC3)C1)c1ccnc(-c2ccccc2)c1] |
| 506 | 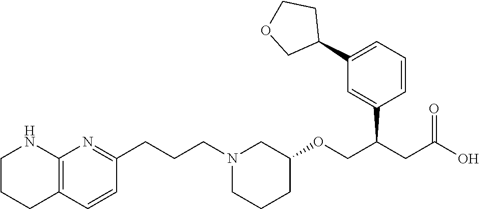 [SMILES: O=C(O)C[C@@H](CO[C@@H]1CCCN(CCCc2ccc3c(n2)NCCC3)C1)c1cccc([C@@H]2CCOC2)c1] |
| 507 | 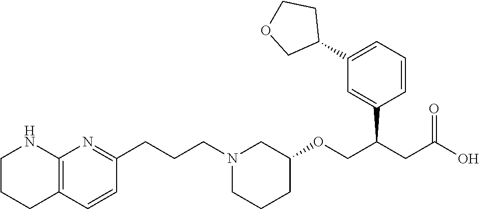 [SMILES: O=C(O)C[C@@H](CO[C@@H]1CCCN(CCCc2ccc3c(n2)NCCC3)C1)c1cccc([C@H]2CCOC2)c1] |
| 508 | 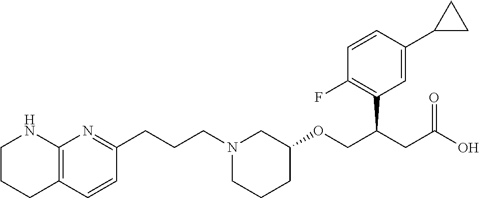 [SMILES: O=C(O)C[C@@H](CO[C@@H]1CCCN(CCCc2ccc3c(n2)NCCC3)C1)c1cc(C2CC2)ccc1F] |
| 509 | 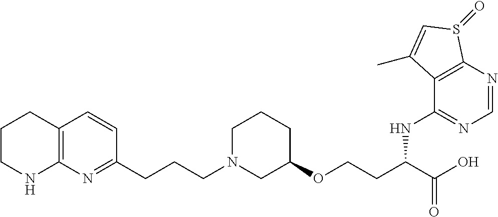 [SMILES: CC1=CS(=O)c2ncnc(N[C@@H](CCO[C@@H]3CCCN(CCCc4ccc5c(n4)NCCC5)C3)C(=O)O)c21] |
| 510 | 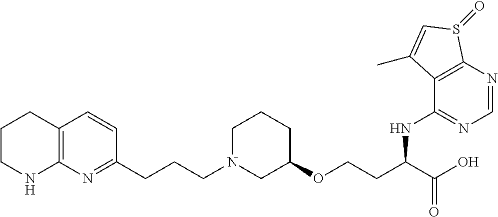 [SMILES: CC1=CS(=O)c2ncnc(N[C@H](CCO[C@@H]3CCCN(CCCc4ccc5c(n4)NCCC5)C3)C(=O)O)c21] |
| 511 | 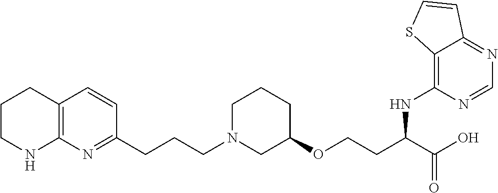 [SMILES: O=C(O)[C@@H](CCO[C@@H]1CCCN(CCCc2ccc3c(n2)NCCC3)C1)Nc1ncnc2ccsc12] |
| 512 | 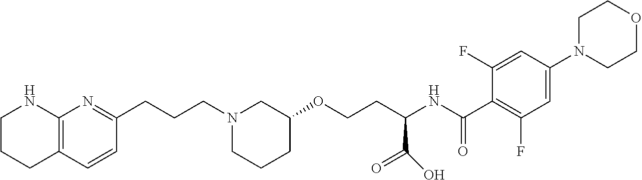 [SMILES: O=C(N[C@H](CCO[C@@H]1CCCN(CCCc2ccc3c(n2)NCCC3)C1)C(=O)O)c1c(F)cc(N2CCOCC2)cc1F] |
| 513 | 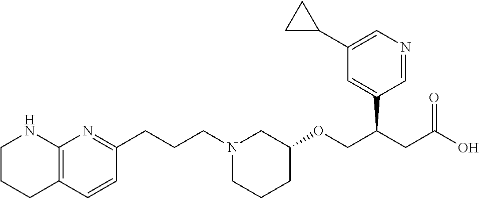 [SMILES: O=C(O)C[C@@H](CO[C@@H]1CCCN(CCCc2ccc3c(n2)NCCC3)C1)c1cncc(C2CC2)c1] |
| 514 | 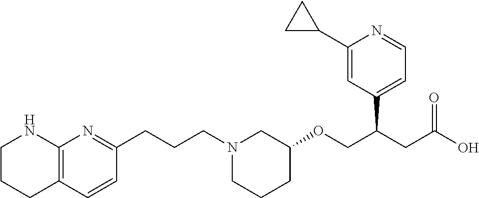 [SMILES: O=C(O)C[C@@H](CO[C@@H]1CCCN(CCCc2ccc3c(n2)NCCC3)C1)c1ccnc(C2CC2)c1] |
| 515 | 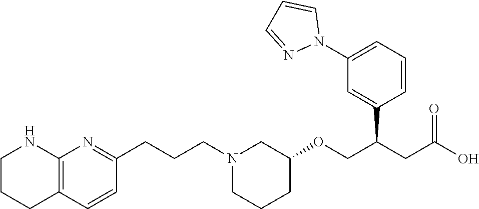 [SMILES: O=C(O)C[C@@H](CO[C@@H]1CCCN(CCCc2ccc3c(n2)NCCC3)C1)c1cccc(-n2cccn2)c1] |
| 516 | 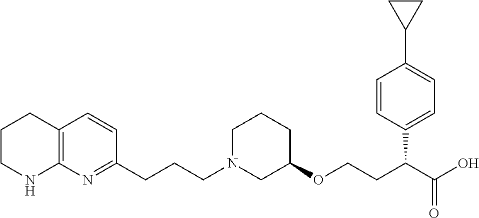 [SMILES: O=C(O)[C@H](CCO[C@@H]1CCCN(CCCc2ccc3c(n2)NCCC3)C1)c1ccc(C2CC2)cc1] |
| 517 | 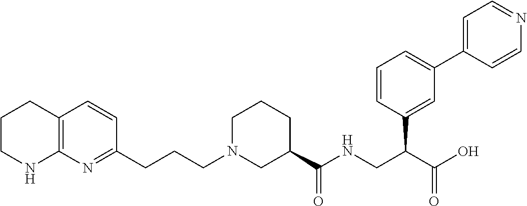 [SMILES: O=C(NC[C@H](C(=O)O)c1cccc(-c2ccncc2)c1)[C@@H]1CCCN(CCCc2ccc3c(n2)NCCC3)C1] |
| 518 | 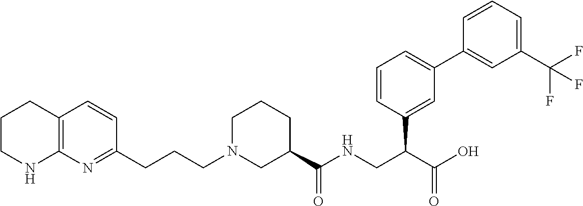 [SMILES: O=C(NC[C@H](C(=O)O)c1cccc(-c2cccc(C(F)(F)F)c2)c1)[C@@H]1CCCN(CCCc2ccc3c(n2)NCCC3)C1] |
| 519 | 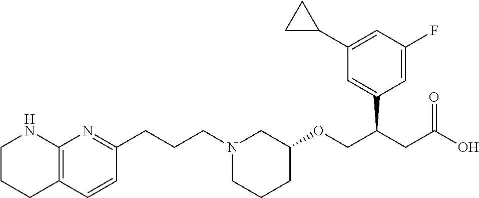 [SMILES: O=C(O)C[C@@H](CO[C@@H]1CCCN(CCCc2ccc3c(n2)NCCC3)C1)c1cc(F)cc(C2CC2)c1] |
| 520 | 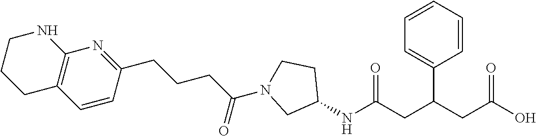 [SMILES: O=C(O)CC(CC(=O)N[C@H]1CCN(C(=O)CCCc2ccc3c(n2)NCCC3)C1)c1ccccc1] |
| 521 | 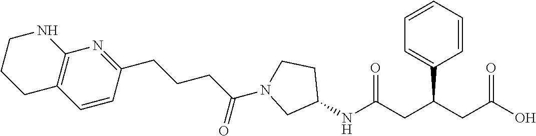 [SMILES: O=C(O)C[C@@H](CC(=O)N[C@H]1CCN(C(=O)CCCc2ccc3c(n2)NCCC3)C1)c1ccccc1] |
| 522 | 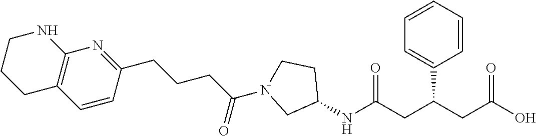 [SMILES: O=C(O)C[C@H](CC(=O)N[C@H]1CCN(C(=O)CCCc2ccc3c(n2)NCCC3)C1)c1ccccc1] |
| 523 | 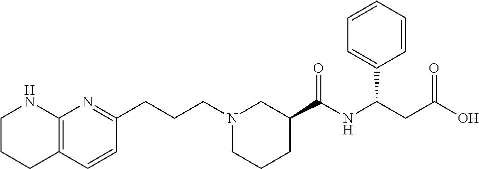 [SMILES: O=C(O)C[C@H](NC(=O)[C@H]1CCCN(CCCc2ccc3c(n2)NCCC3)C1)c1ccccc1] |
| 524 | 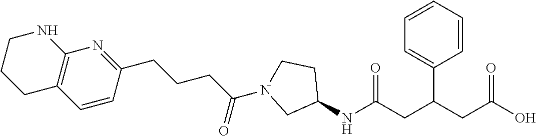 [SMILES: O=C(O)CC(CC(=O)N[C@@H]1CCN(C(=O)CCCc2ccc3c(n2)NCCC3)C1)c1ccccc1] |
| 525 | 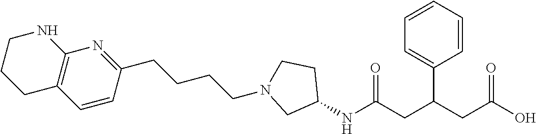 [SMILES: O=C(O)CC(CC(=O)N[C@H]1CCN(CCCCc2ccc3c(n2)NCCC3)C1)c1ccccc1] |
| 526 | 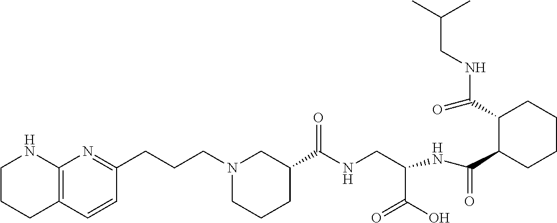 [SMILES: CC(C)CNC(=O)[C@@H]1CCCC[C@H]1C(=O)N[C@@H](CNC(=O)[C@@H]1CCCN(CCCc2ccc3c(n2)NCCC3)C1)C(=O)O] |
| 527 | 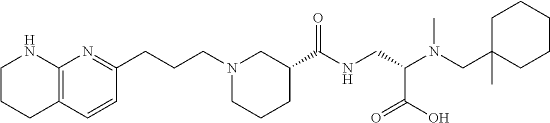 [SMILES: CN(CC1(C)CCCCC1)[C@@H](CNC(=O)[C@@H]1CCCN(CCCc2ccc3c(n2)NCCC3)C1)C(=O)O] |
| 528 | 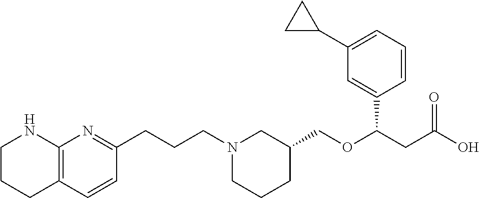 [SMILES: O=C(O)C[C@H](OC[C@@H]1CCCN(CCCc2ccc3c(n2)NCCC3)C1)c1cccc(C2CC2)c1] |
| 529 | 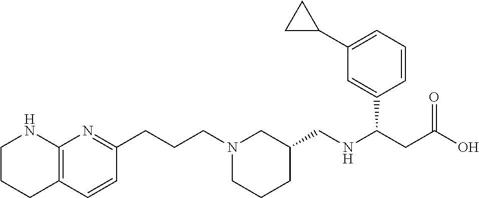 [SMILES: O=C(O)C[C@H](NC[C@@H]1CCCN(CCCc2ccc3c(n2)NCCC3)C1)c1cccc(C2CC2)c1] |
| 530 | 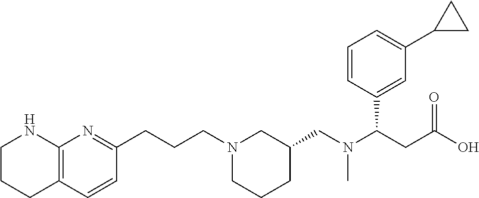 [SMILES: CN(C[C@@H]1CCCN(CCCc2ccc3c(n2)NCCC3)C1)[C@@H](CC(=O)O)c1cccc(C2CC2)c1] |
| 531 | 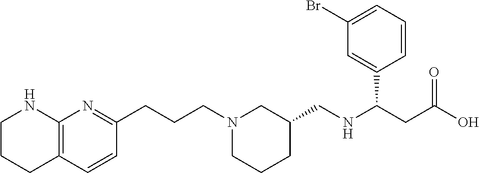 [SMILES: O=C(O)C[C@H](NC[C@@H]1CCCN(CCCc2ccc3c(n2)NCCC3)C1)c1cccc(Br)c1] |
| 532 | 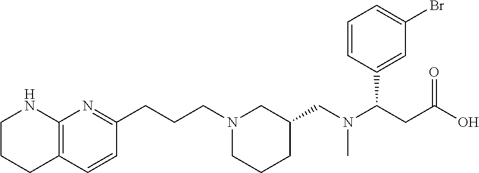 [SMILES: CN(C[C@@H]1CCCN(CCCc2ccc3c(n2)NCCC3)C1)[C@@H](CC(=O)O)c1cccc(Br)c1] |
| 533 | 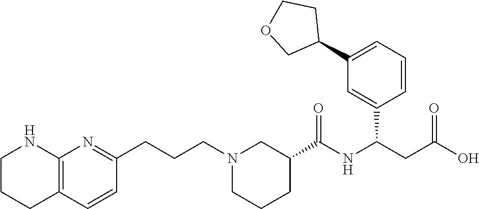 [SMILES: O=C(O)C[C@H](NC(=O)[C@@H]1CCCN(CCCc2ccc3c(n2)NCCC3)C1)c1cccc([C@@H]2CCOC2)c1] |
| 534 | 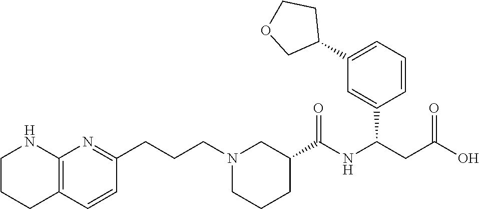 [SMILES: O=C(O)C[C@H](NC(=O)[C@@H]1CCCN(CCCc2ccc3c(n2)NCCC3)C1)c1cccc([C@H]2CCOC2)c1] |
| 535 | 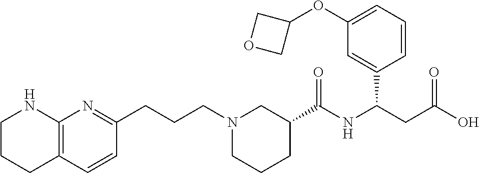 [SMILES: O=C(O)C[C@H](NC(=O)[C@@H]1CCCN(CCCc2ccc3c(n2)NCCC3)C1)c1cccc(OC2COC2)c1] |
| 536 | 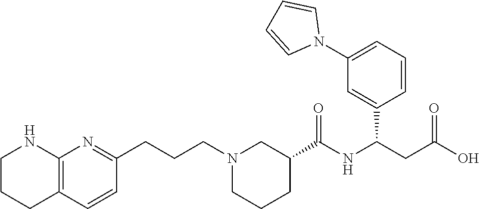 [SMILES: O=C(O)C[C@H](NC(=O)[C@@H]1CCCN(CCCc2ccc3c(n2)NCCC3)C1)c1cccc(-n2cccc2)c1] |
| 537 | 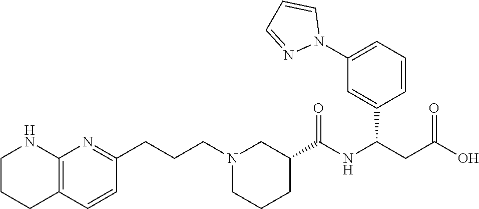 [SMILES: O=C(O)C[C@H](NC(=O)[C@@H]1CCCN(CCCc2ccc3c(n2)NCCC3)C1)c1cccc(-n2cccn2)c1] |
| 538 | 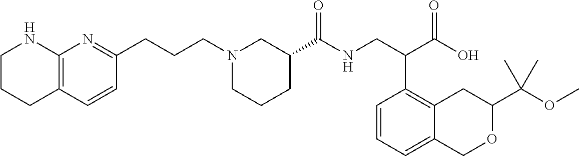 [SMILES: COC(C)(C)C1Cc2c(cccc2C(CNC(=O)[C@@H]2CCCN(CCCc3ccc4c(n3)NCCC4)C2)C(=O)O)CO1] |
| 539 | 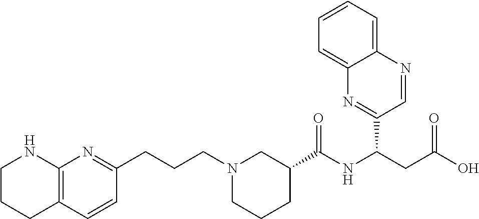 [SMILES: O=C(O)C[C@H](NC(=O)[C@@H]1CCCN(CCCc2ccc3c(n2)NCCC3)C1)c1cnc2ccccc2n1] |
| 540 | 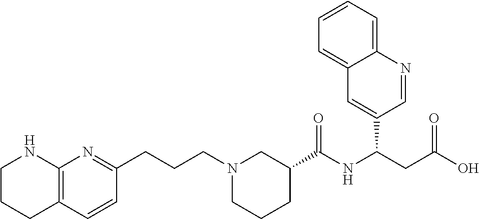 [SMILES: O=C(O)C[C@H](NC(=O)[C@@H]1CCCN(CCCc2ccc3c(n2)NCCC3)C1)c1cnc2ccccc2c1] |
| 541 | 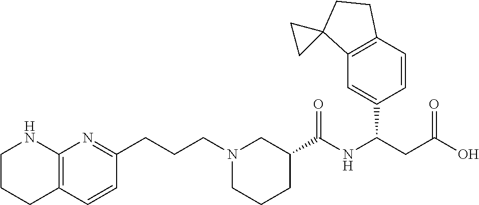 [SMILES: O=C(O)C[C@H](NC(=O)[C@@H]1CCCN(CCCc2ccc3c(n2)NCCC3)C1)c1ccc2c(c1)C1(CC2)CC1] |
| 542 | 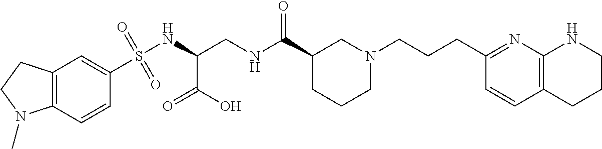 [SMILES: CN1CCc2cc(S(=O)(=O)N[C@@H](CNC(=O)[C@@H]3CCCN(CCCc4ccc5c(n4)NCCC5)C3)C(=O)O)ccc21] |
| 543 | 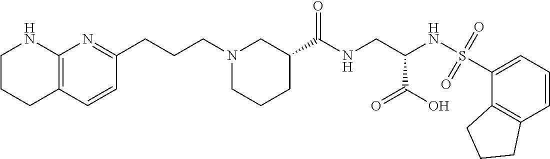 [SMILES: O=C(NC[C@H](NS(=O)(=O)c1cccc2c1CCC2)C(=O)O)[C@@H]1CCCN(CCCc2ccc3c(n2)NCCC3)C1] |
| 544 | 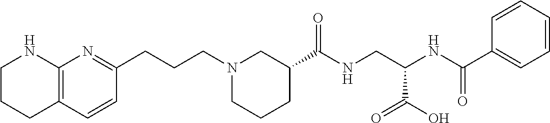 [SMILES: O=C(N[C@@H](CNC(=O)[C@@H]1CCCN(CCCc2ccc3c(n2)NCCC3)C1)C(=O)O)c1ccccc1] |
| 545 | 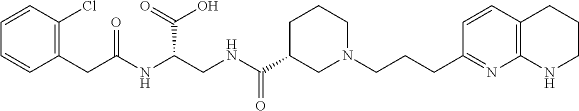 [SMILES: O=C(Cc1ccccc1Cl)N[C@@H](CNC(=O)[C@@H]1CCCN(CCCc2ccc3c(n2)NCCC3)C1)C(=O)O] |
| 546 | 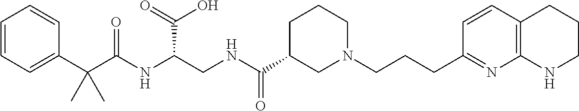 [SMILES: CC(C)(C(=O)N[C@@H](CNC(=O)[C@@H]1CCCN(CCCc2ccc3c(n2)NCCC3)C1)C(=O)O)c1ccccc1] |
| 547 | 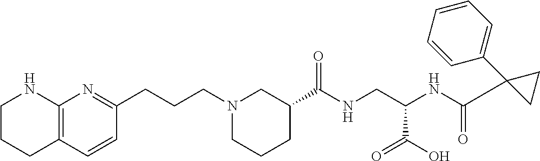 [SMILES: O=C(NC[C@H](NC(=O)C1(c2ccccc2)CC1)C(=O)O)[C@@H]1CCCN(CCCc2ccc3c(n2)NCCC3)C1] |
| 548 | 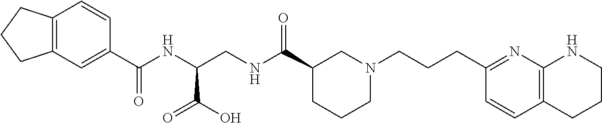 [SMILES: O=C(N[C@@H](CNC(=O)[C@@H]1CCCN(CCCc2ccc3c(n2)NCCC3)C1)C(=O)O)c1ccc2c(c1)CCC2] |
| 549 | 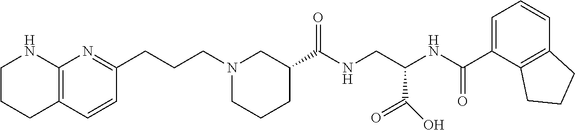 [SMILES: O=C(N[C@@H](CNC(=O)[C@@H]1CCCN(CCCc2ccc3c(n2)NCCC3)C1)C(=O)O)c1cccc2c1CCC2] |
| 550 | 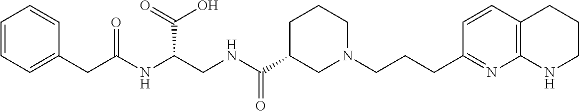 [SMILES: O=C(Cc1ccccc1)N[C@@H](CNC(=O)[C@@H]1CCCN(CCCc2ccc3c(n2)NCCC3)C1)C(=O)O] |
| 551 | 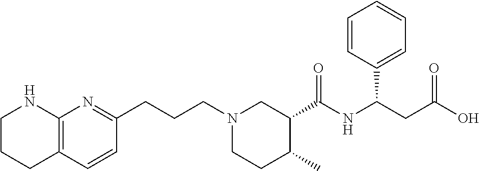 [SMILES: C[C@@H]1CCN(CCCc2ccc3c(n2)NCCC3)C[C@@H]1C(=O)N[C@@H](CC(=O)O)c1ccccc1] |
| 552 | 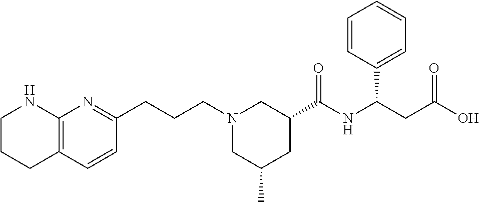 [SMILES: C[C@H]1C[C@@H](C(=O)N[C@@H](CC(=O)O)c2ccccc2)CN(CCCc2ccc3c(n2)NCCC3)C1] |
| 553 | 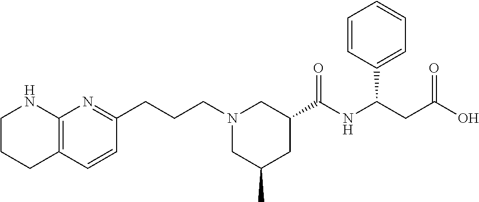 [SMILES: C[C@@H]1C[C@@H](C(=O)N[C@@H](CC(=O)O)c2ccccc2)CN(CCCc2ccc3c(n2)NCCC3)C1] |
| 554 | 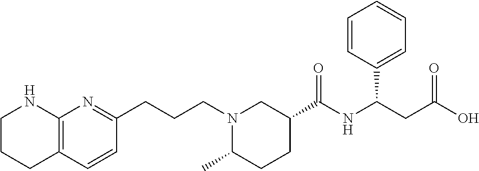 [SMILES: C[C@H]1CC[C@@H](C(=O)N[C@@H](CC(=O)O)c2ccccc2)CN1CCCc1ccc2c(n1)NCCC2] |
| 555 | 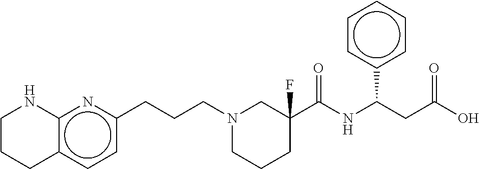 |
| 556 | 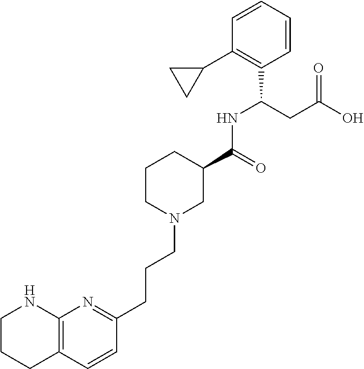 [SMILES: O=C(O)C[C@H](NC(=O)[C@@H]1CCCN(CCCc2ccc3c(n2)NCCC3)C1)c1ccccc1C1CC1] |
| 557 | 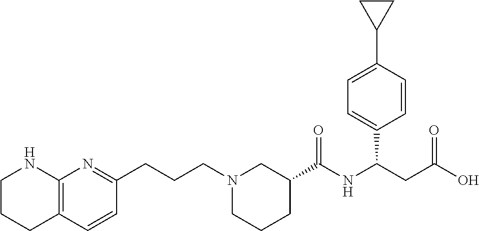 [SMILES: O=C(O)C[C@H](NC(=O)[C@@H]1CCCN(CCCc2ccc3c(n2)NCCC3)C1)c1ccc(C2CC2)cc1] |
| 558 | 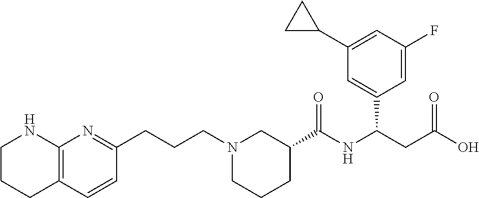 [SMILES: O=C(O)C[C@H](NC(=O)[C@@H]1CCCN(CCCc2ccc3c(n2)NCCC3)C1)c1cc(F)cc(C2CC2)c1] |
| 559 | 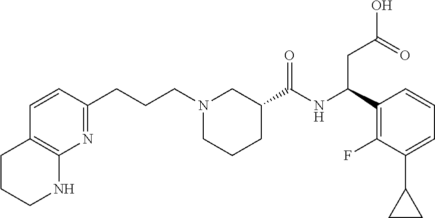 [SMILES: O=C(O)C[C@H](NC(=O)[C@@H]1CCCN(CCCc2ccc3c(n2)NCCC3)C1)c1cccc(C2CC2)c1F] |
| 560 | 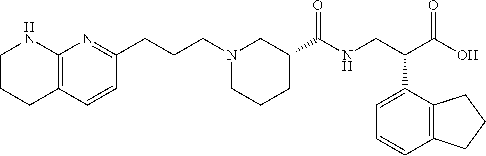 [SMILES: O=C(NC[C@H](C(=O)O)c1cccc2c1CCC2)[C@@H]1CCCN(CCCc2ccc3c(n2)NCCC3)C1] |
| 561 | 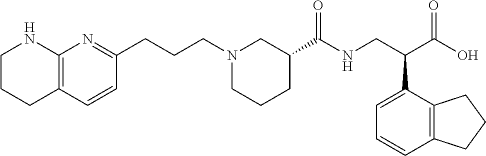 [SMILES: O=C(NC[C@@H](C(=O)O)c1cccc2c1CCC2)[C@@H]1CCCN(CCCc2ccc3c(n2)NCCC3)C1] |
| 562 | 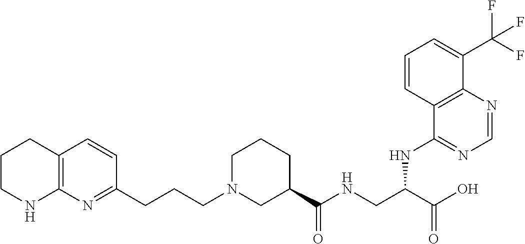 [SMILES: O=C(NC[C@H](Nc1ncnc2c(C(F)(F)F)cccc12)C(=O)O)[C@@H]1CCCN(CCCc2ccc3c(n2)NCCC3)C1] |
| 563 | 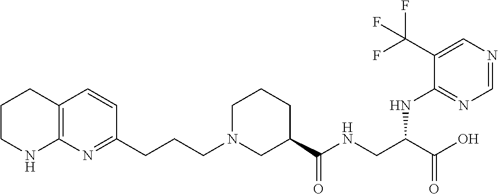 [SMILES: O=C(NC[C@H](Nc1ncncc1C(F)(F)F)C(=O)O)[C@@H]1CCCN(CCCc2ccc3c(n2)NCCC3)C1] |
| 564 | 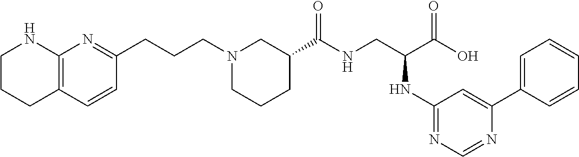 [SMILES: O=C(NC[C@H](Nc1cc(-c2ccccc2)ncn1)C(=O)O)[C@@H]1CCCN(CCCc2ccc3c(n2)NCCC3)C1] |
| 565 | 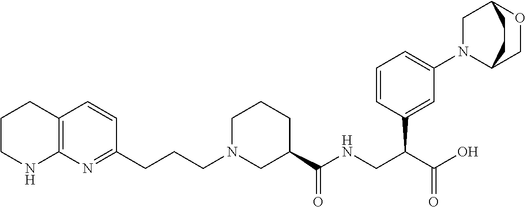 [SMILES: O=C(NC[C@H](C(=O)O)c1cccc(N2C[C@H]3CC[C@@H]2CO3)c1)[C@@H]1CCCN(CCCc2ccc3c(n2)NCCC3)C1] |
| 566 | 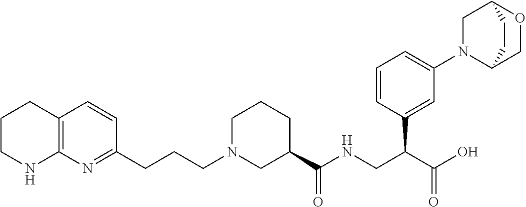 [SMILES: O=C(NC[C@H](C(=O)O)c1cccc(N2C[C@@H]3CC[C@H]2CO3)c1)[C@@H]1CCCN(CCCc2ccc3c(n2)NCCC3)C1] |
| 567 | 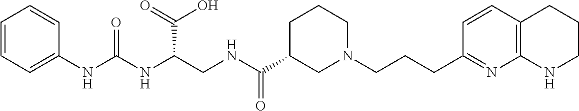 [SMILES: O=C(Nc1ccccc1)N[C@@H](CNC(=O)[C@@H]1CCCN(CCCc2ccc3c(n2)NCCC3)C1)C(=O)O] |
| 568 | 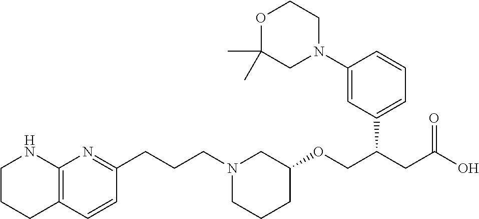 [SMILES: CC1(C)CN(c2cccc([C@@H](CO[C@@H]3CCCN(CCCc4ccc5c(n4)NCCC5)C3)CC(=O)O)c2)CCO1] |
| 569 | 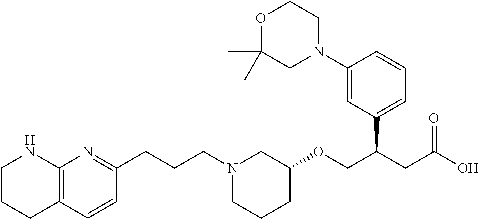 [SMILES: CC1(C)CN(c2cccc([C@H](CO[C@@H]3CCCN(CCCc4ccc5c(n4)NCCC5)C3)CC(=O)O)c2)CCO1] |
| 570 | 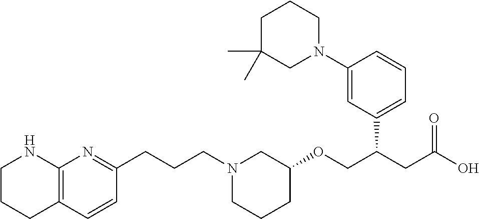 [SMILES: CC1(C)CCCN(c2cccc([C@@H](CO[C@@H]3CCCN(CCCc4ccc5c(n4)NCCC5)C3)CC(=O)O)c2)C1] |
| 571 | 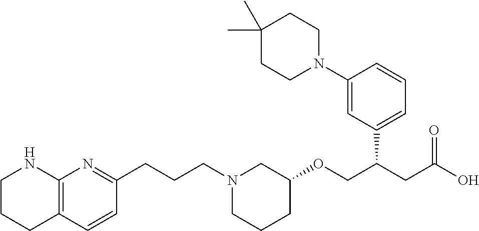 [SMILES: CC1(C)CCN(c2cccc([C@@H](CO[C@@H]3CCCN(CCCc4ccc5c(n4)NCCC5)C3)CC(=O)O)c2)CC1] |
| 572 | 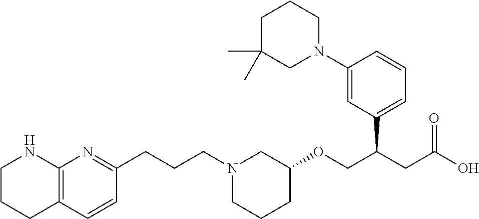 [SMILES: CC1(C)CCCN(c2cccc([C@H](CO[C@@H]3CCCN(CCCc4ccc5c(n4)NCCC5)C3)CC(=O)O)c2)C1] |
| 573 | 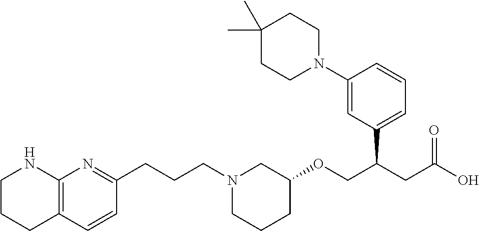 [SMILES: CC1(C)CCN(c2cccc([C@H](CO[C@@H]3CCCN(CCCc4ccc5c(n4)NCCC5)C3)CC(=O)O)c2)CC1] |
| 574 | 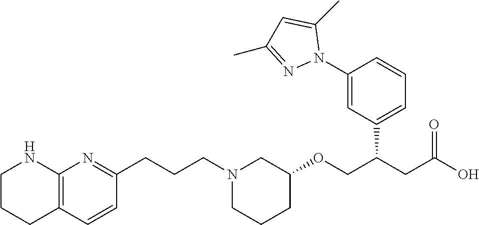 [SMILES: Cc1cc(C)n(-c2cccc([C@@H](CO[C@@H]3CCCN(CCCc4ccc5c(n4)NCCC5)C3)CC(=O)O)c2)n1] |
| 575 | 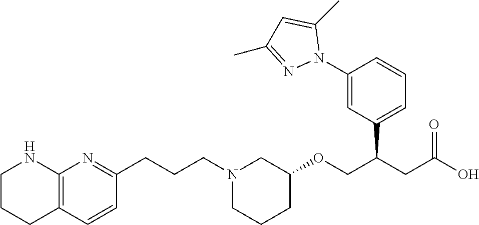 [SMILES: Cc1cc(C)n(-c2cccc([C@H](CO[C@@H]3CCCN(CCCc4ccc5c(n4)NCCC5)C3)CC(=O)O)c2)n1] |
| 576 | 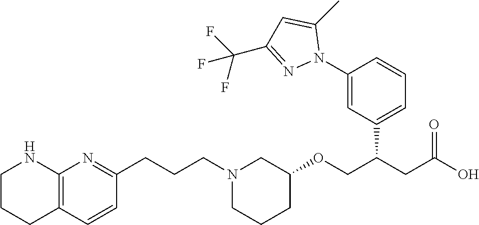 [SMILES: Cc1cc(C(F)(F)F)nn1-c1cccc([C@@H](CO[C@@H]2CCCN(CCCc3ccc4c(n3)NCCC4)C2)CC(=O)O)c1] |
| 577 | 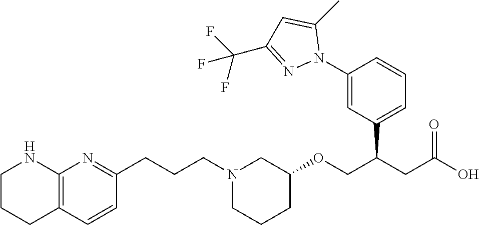 [SMILES: Cc1cc(C(F)(F)F)nn1-c1cccc([C@H](CO[C@@H]2CCCN(CCCc3ccc4c(n3)NCCC4)C2)CC(=O)O)c1] |
| 578 | 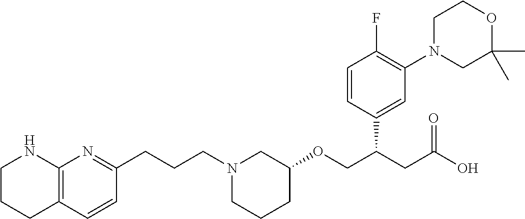 [SMILES: CC1(C)CN(c2cc([C@@H](CO[C@@H]3CCCN(CCCc4ccc5c(n4)NCCC5)C3)CC(=O)O)ccc2F)CCO1] |
| 579 | 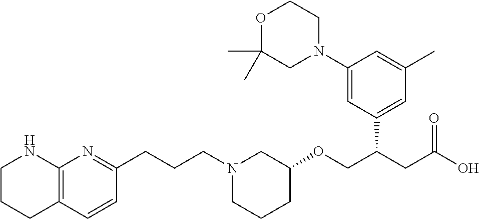 [SMILES: Cc1cc([C@@H](CO[C@@H]2CCCN(CCCc3ccc4c(n3)NCCC4)C2)CC(=O)O)cc(N2CCOC(C)(C)C2)c1] |
| 580 | 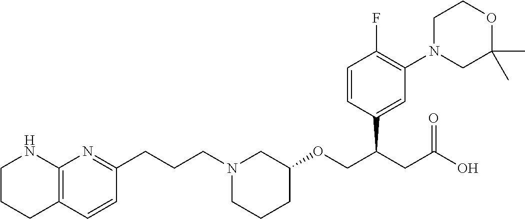 [SMILES: CC1(C)CN(c2cc([C@H](CO[C@@H]3CCCN(CCCc4ccc5c(n4)NCCC5)C3)CC(=O)O)ccc2F)CCO1] |
| 581 | 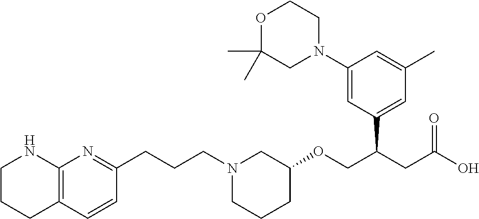 [SMILES: Cc1cc([C@H](CO[C@@H]2CCCN(CCCc3ccc4c(n3)NCCC4)C2)CC(=O)O)cc(N2CCOC(C)(C)C2)c1] |
| 582 | 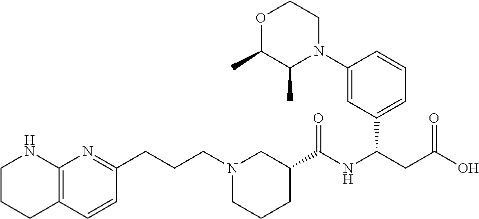 [SMILES: C[C@H]1OCCN(c2cccc([C@H](CC(=O)O)NC(=O)[C@@H]3CCCN(CCCc4ccc5c(n4)NCCC5)C3)c2)[C@H]1C] |
| 583 | 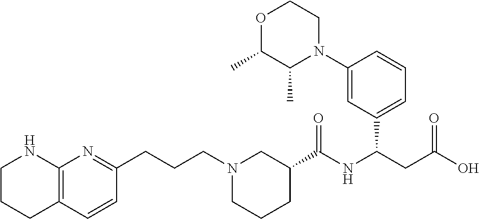 [SMILES: C[C@@H]1OCCN(c2cccc([C@H](CC(=O)O)NC(=O)[C@@H]3CCCN(CCCc4ccc5c(n4)NCCC5)C3)c2)[C@@H]1C] |
| 584 | 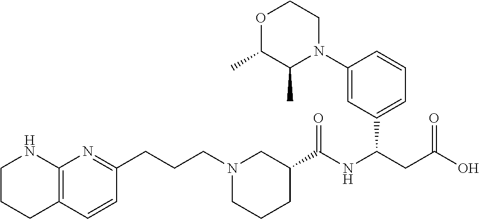 [SMILES: C[C@@H]1OCCN(c2cccc([C@H](CC(=O)O)NC(=O)[C@@H]3CCCN(CCCc4ccc5c(n4)NCCC5)C3)c2)[C@H]1C] |
| 585 | 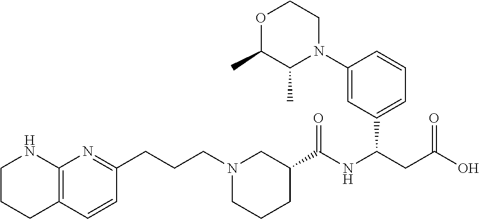 [SMILES: C[C@@H]1[C@@H](C)OCCN1c1cccc([C@H](CC(=O)O)NC(=O)[C@@H]2CCCN(CCCc3ccc4c(n3)NCCC4)C2)c1] |
| 586 | 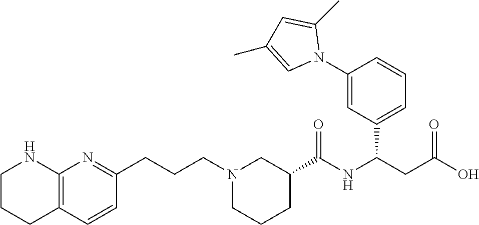 [SMILES: Cc1cc(C)n(-c2cccc([C@H](CC(=O)O)NC(=O)[C@@H]3CCCN(CCCc4ccc5c(n4)NCCC5)C3)c2)c1] |
| 587 | 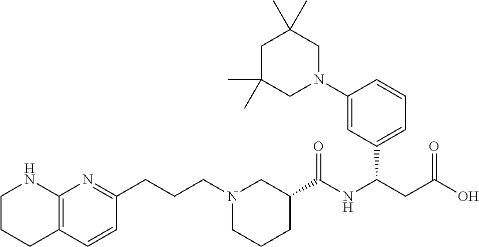 [SMILES: CC1(C)CN(c2cccc([C@H](CC(=O)O)NC(=O)[C@@H]3CCCN(CCCc4ccc5c(n4)NCCC5)C3)c2)CC(C)(C)C1] |
| 588 | 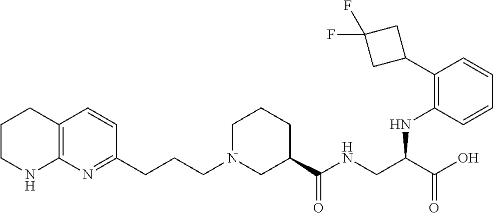 [SMILES: O=C(NC[C@@H](Nc1ccccc1C1CC(F)(F)C1)C(=O)O)[C@@H]1CCCN(CCCc2ccc3c(n2)NCCC3)C1] |
| 589 | 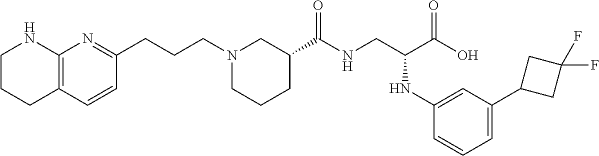 [SMILES: O=C(NC[C@@H](Nc1cccc(C2CC(F)(F)C2)c1)C(=O)O)[C@@H]1CCCN(CCCc2ccc3c(n2)NCCC3)C1] |
| 590 | 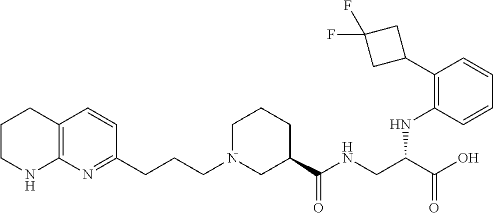 [SMILES: O=C(NC[C@H](Nc1ccccc1C1CC(F)(F)C1)C(=O)O)[C@@H]1CCCN(CCCc2ccc3c(n2)NCCC3)C1] |
| 591 | 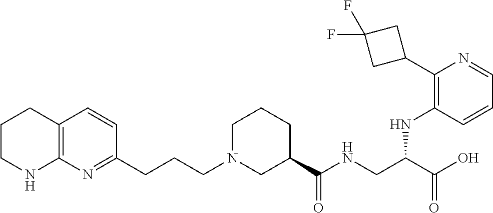 [SMILES: O=C(NC[C@H](Nc1cccnc1C1CC(F)(F)C1)C(=O)O)[C@@H]1CCCN(CCCc2ccc3c(n2)NCCC3)C1] |
| 592 | 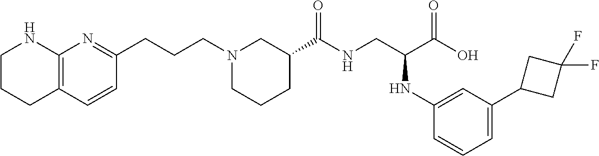 [SMILES: O=C(NC[C@H](Nc1cccc(C2CC(F)(F)C2)c1)C(=O)O)[C@@H]1CCCN(CCCc2ccc3c(n2)NCCC3)C1] |
| 593 | 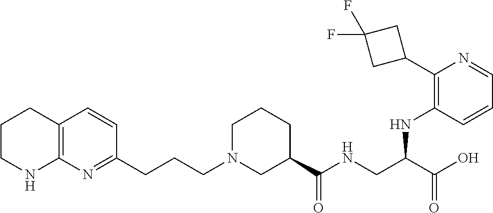 [SMILES: O=C(NC[C@@H](Nc1cccnc1C1CC(F)(F)C1)C(=O)O)[C@@H]1CCCN(CCCc2ccc3c(n2)NCCC3)C1] |
| 594 | 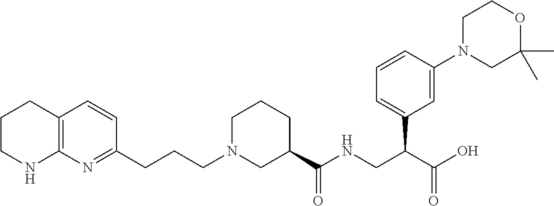 [SMILES: CC1(C)CN(c2cccc([C@H](CNC(=O)[C@@H]3CCCN(CCCc4ccc5c(n4)NCCC5)C3)C(=O)O)c2)CCO1] |
| 595 | 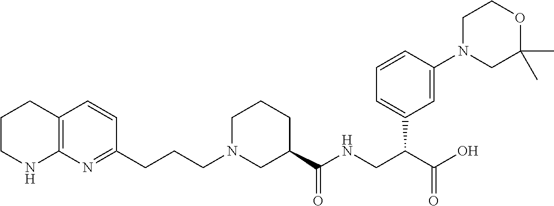 [SMILES: CC1(C)CN(c2cccc([C@@H](CNC(=O)[C@@H]3CCCN(CCCc4ccc5c(n4)NCCC5)C3)C(=O)O)c2)CCO1] |
| 596 | 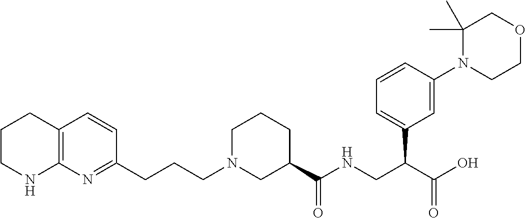 [SMILES: CC1(C)COCCN1c1cccc([C@H](CNC(=O)[C@@H]2CCCN(CCCc3ccc4c(n3)NCCC4)C2)C(=O)O)c1] |
| 597 | 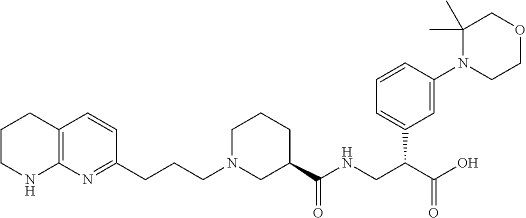 [SMILES: CC1(C)COCCN1c1cccc([C@@H](CNC(=O)[C@@H]2CCCN(CCCc3ccc4c(n3)NCCC4)C2)C(=O)O)c1] |
| 598 | 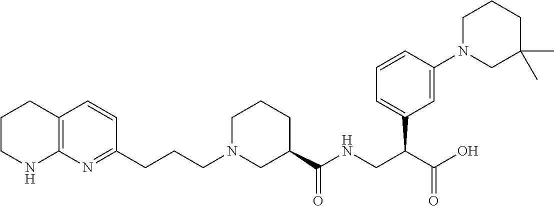 [SMILES: CC1(C)CCCN(c2cccc([C@H](CNC(=O)[C@@H]3CCCN(CCCc4ccc5c(n4)NCCC5)C3)C(=O)O)c2)C1] |
| 599 | 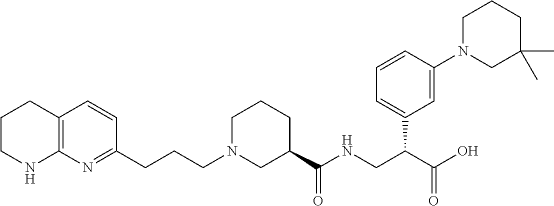 [SMILES: CC1(C)CCCN(c2cccc([C@@H](CNC(=O)[C@@H]3CCCN(CCCc4ccc5c(n4)NCCC5)C3)C(=O)O)c2)C1] |
| 600 | 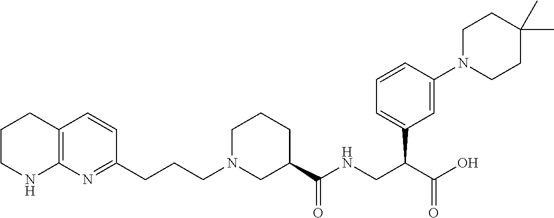 [SMILES: CC1(C)CCN(c2cccc([C@H](CNC(=O)[C@@H]3CCCN(CCCc4ccc5c(n4)NCCC5)C3)C(=O)O)c2)CC1] |
| 601 | 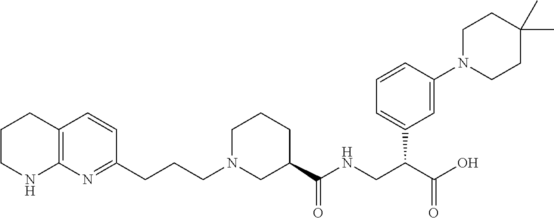 [SMILES: CC1(C)CCN(c2cccc([C@@H](CNC(=O)[C@@H]3CCCN(CCCc4ccc5c(n4)NCCC5)C3)C(=O)O)c2)CC1] |
| 602 | 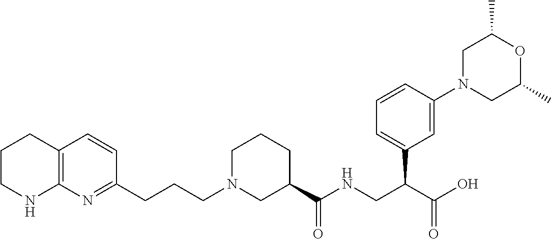 [SMILES: C[C@@H]1CN(c2cccc([C@H](CNC(=O)[C@@H]3CCCN(CCCc4ccc5c(n4)NCCC5)C3)C(=O)O)c2)C[C@H](C)O1] |
| 603 | 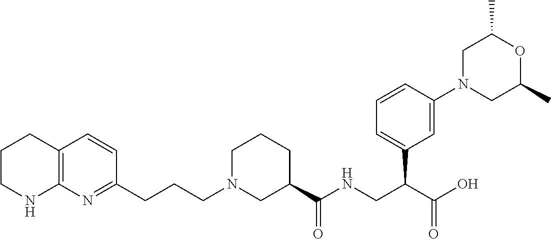 [SMILES: C[C@H]1CN(c2cccc([C@H](CNC(=O)[C@@H]3CCCN(CCCc4ccc5c(n4)NCCC5)C3)C(=O)O)c2)C[C@H](C)O1] |
| 604 | 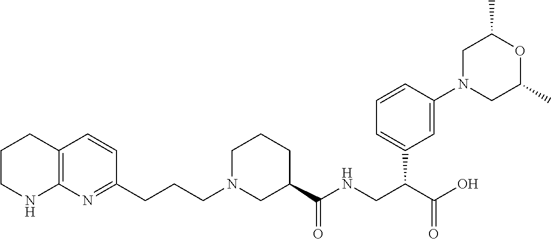 [SMILES: C[C@@H]1CN(c2cccc([C@@H](CNC(=O)[C@@H]3CCCN(CCCc4ccc5c(n4)NCCC5)C3)C(=O)O)c2)C[C@H](C)O1] |
| 605 | 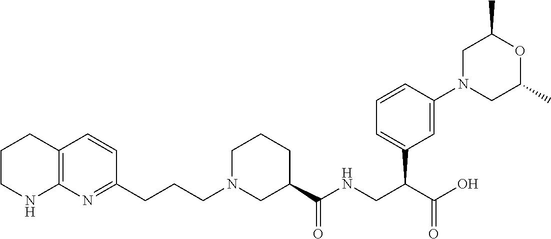 [SMILES: C[C@@H]1CN(c2cccc([C@H](CNC(=O)[C@@H]3CCCN(CCCc4ccc5c(n4)NCCC5)C3)C(=O)O)c2)C[C@@H](C)O1] |
| 606 | 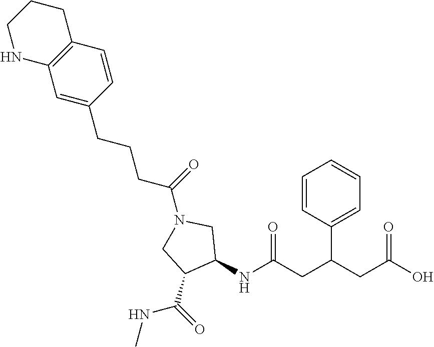 [SMILES: CNC(=O)[C@@H]1CN(C(=O)CCCc2ccc3c(c2)NCCC3)C[C@H]1NC(=O)CC(CC(=O)O)c1ccccc1] |
| 607 | 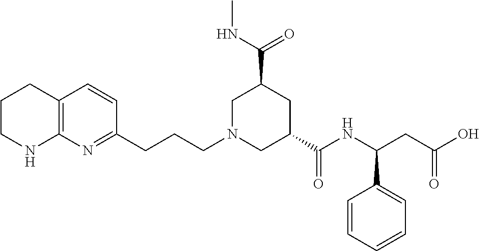 [SMILES: CNC(=O)[C@H]1C[C@H](C(=O)N[C@@H](CC(=O)O)c2ccccc2)CN(CCCc2ccc3c(n2)NCCC3)C1] |
| 608 | 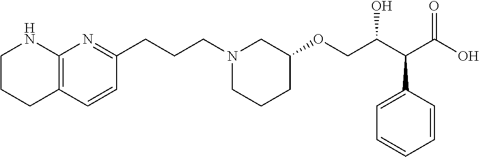 [SMILES: O=C(O)[C@@H](c1ccccc1)[C@@H](O)CO[C@@H]1CCCN(CCCc2ccc3c(n2)NCCC3)C1] |
| 609 | 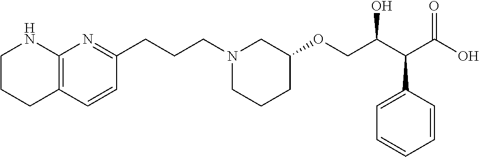 [SMILES: O=C(O)[C@@H](c1ccccc1)[C@H](O)CO[C@@H]1CCCN(CCCc2ccc3c(n2)NCCC3)C1] |
| 610 | 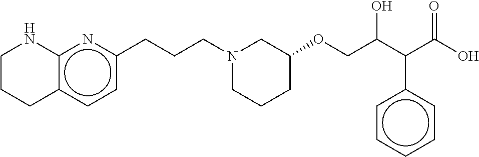 |
| 611 | 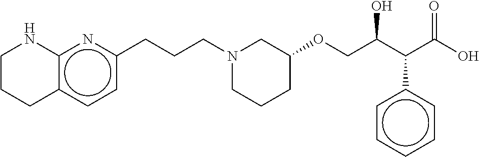 |
| 612 | 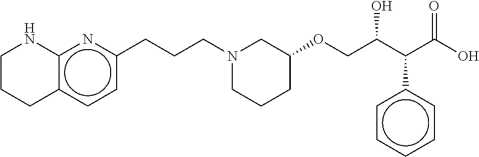 |
| 613 | 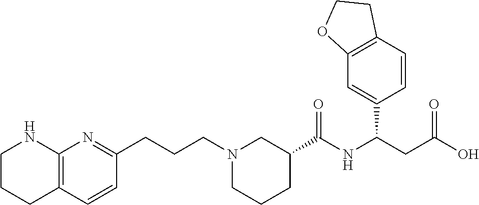 [SMILES: O=C(O)C[C@H](NC(=O)[C@@H]1CCCN(CCCc2ccc3c(n2)NCCC3)C1)c1ccc2c(c1)OCC2] |
| 614 | 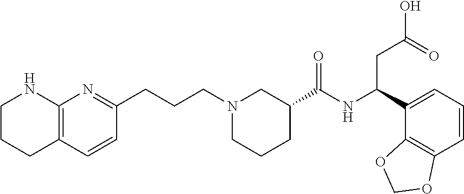 [SMILES: O=C(O)C[C@H](NC(=O)[C@@H]1CCCN(CCCc2ccc3c(n2)NCCC3)C1)c1cccc2c1OCO2] |
| 615 | 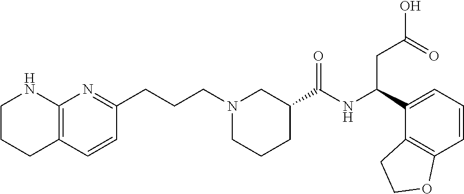 [SMILES: O=C(O)C[C@H](NC(=O)[C@@H]1CCCN(CCCc2ccc3c(n2)NCCC3)C1)c1cccc2c1CCO2] |
| 616 | 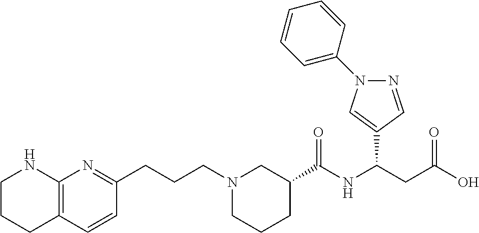 [SMILES: O=C(O)C[C@H](NC(=O)[C@@H]1CCCN(CCCc2ccc3c(n2)NCCC3)C1)c1cnn(-c2ccccc2)c1] |
| 617 | 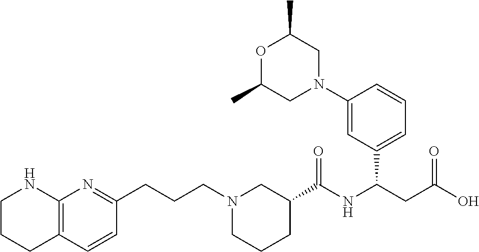 [SMILES: C[C@@H]1CN(c2cccc([C@H](CC(=O)O)NC(=O)[C@@H]3CCCN(CCCc4ccc5c(n4)NCCC5)C3)c2)C[C@H](C)O1] |
| 618 | 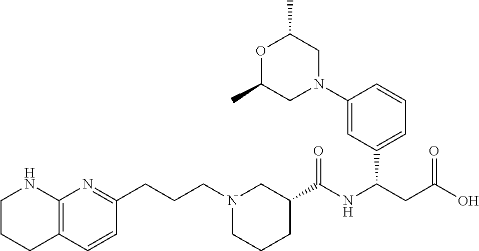 [SMILES: C[C@@H]1CN(c2cccc([C@H](CC(=O)O)NC(=O)[C@@H]3CCCN(CCCc4ccc5c(n4)NCCC5)C3)c2)C[C@@H](C)O1] |
| 619 | 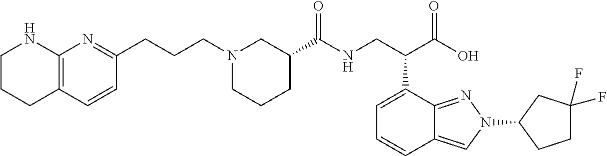 [SMILES: O=C(NC[C@H](C(=O)O)c1cccc2cn([C@H]3CCC(F)(F)C3)nc12)[C@@H]1CCCN(CCCc2ccc3c(n2)NCCC3)C1] |
| 620 | 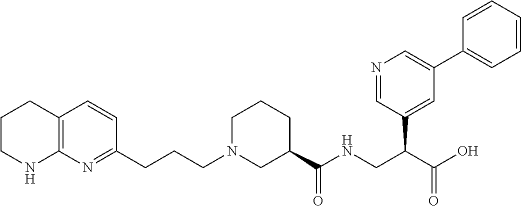 [SMILES: O=C(NC[C@H](C(=O)O)c1cncc(-c2ccccc2)c1)[C@@H]1CCCN(CCCc2ccc3c(n2)NCCC3)C1] |
| 621 | 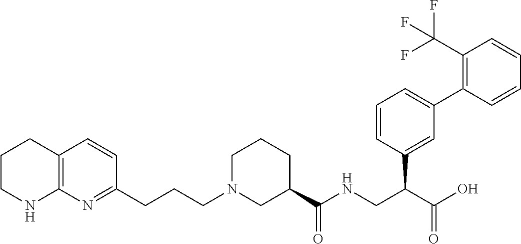 [SMILES: O=C(NC[C@H](C(=O)O)c1cccc(-c2ccccc2C(F)(F)F)c1)[C@@H]1CCCN(CCCc2ccc3c(n2)NCCC3)C1] |
| 622 | 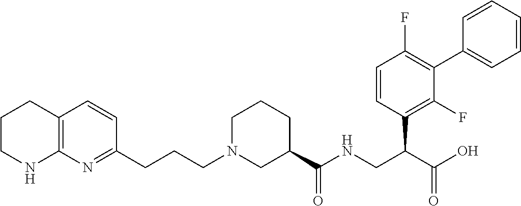 [SMILES: O=C(NC[C@H](C(=O)O)c1ccc(F)c(-c2ccccc2)c1F)[C@@H]1CCCN(CCCc2ccc3c(n2)NCCC3)C1] |
| 623 | 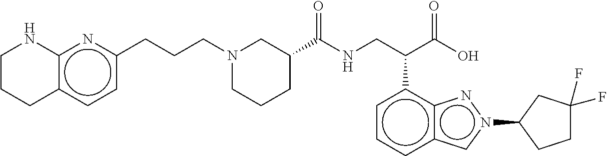 |
| 624 | 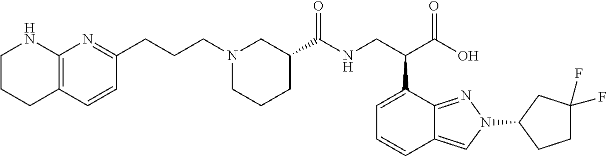 [SMILES: O=C(NC[C@@H](C(=O)O)c1cccc2cn([C@H]3CCC(F)(F)C3)nc12)[C@@H]1CCCN(CCCc2ccc3c(n2)NCCC3)C1] |
| 625 | 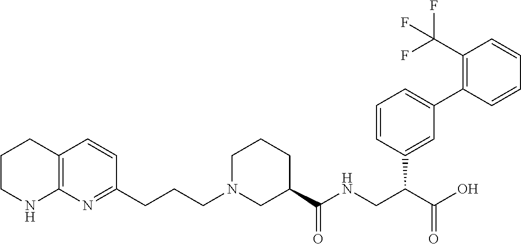 [SMILES: O=C(NC[C@@H](C(=O)O)c1cccc(-c2ccccc2C(F)(F)F)c1)[C@@H]1CCCN(CCCc2ccc3c(n2)NCCC3)C1] |
| 626 | 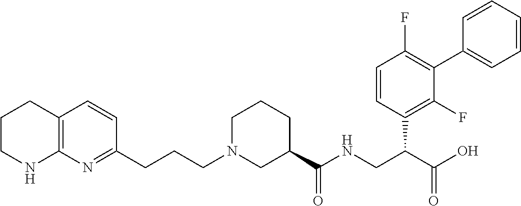 [SMILES: O=C(NC[C@@H](C(=O)O)c1ccc(F)c(-c2ccccc2)c1F)[C@@H]1CCCN(CCCc2ccc3c(n2)NCCC3)C1] |
| 627 | 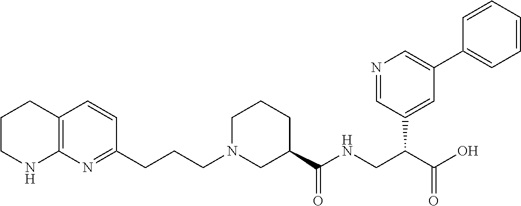 [SMILES: O=C(NC[C@@H](C(=O)O)c1cncc(-c2ccccc2)c1)[C@@H]1CCCN(CCCc2ccc3c(n2)NCCC3)C1] |
| 628 | 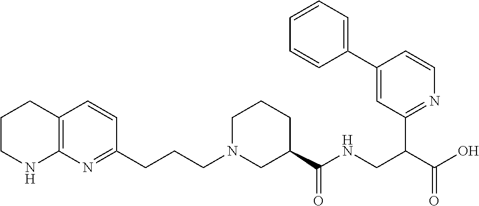 [SMILES: O=C(O)C(CNC(=O)[C@@H]1CCCN(CCCc2ccc3c(n2)NCCC3)C1)c1cc(-c2ccccc2)ccn1] |
| 629 | 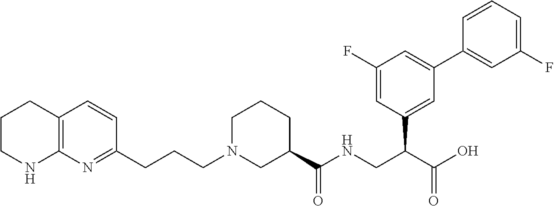 [SMILES: O=C(NC[C@H](C(=O)O)c1cc(F)cc(-c2cccc(F)c2)c1)[C@@H]1CCCN(CCCc2ccc3c(n2)NCCC3)C1] |
| 630 | 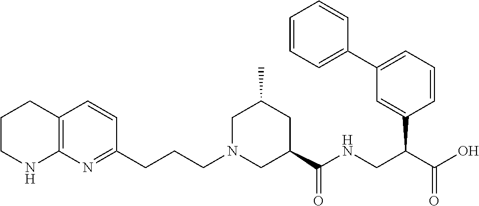 [SMILES: C[C@@H]1C[C@@H](C(=O)NC[C@H](C(=O)O)c2cccc(-c3ccccc3)c2)CN(CCCc2ccc3c(n2)NCCC3)C1] |
| 631 | 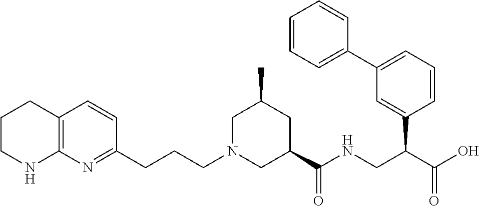 [SMILES: C[C@H]1C[C@@H](C(=O)NC[C@H](C(=O)O)c2cccc(-c3ccccc3)c2)CN(CCCc2ccc3c(n2)NCCC3)C1] |
| 632 | 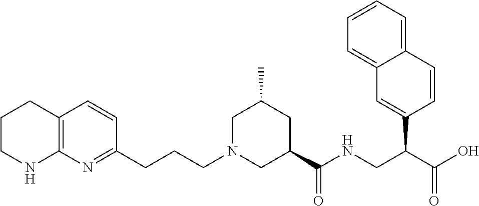 [SMILES: C[C@@H]1C[C@@H](C(=O)NC[C@H](C(=O)O)c2ccc3ccccc3c2)CN(CCCc2ccc3c(n2)NCCC3)C1] |
| 633 | 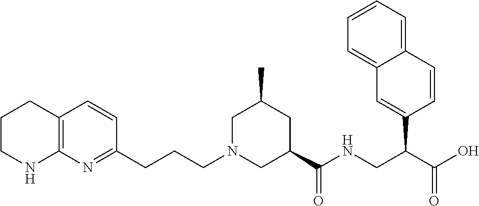 [SMILES: C[C@H]1C[C@@H](C(=O)NC[C@H](C(=O)O)c2ccc3ccccc3c2)CN(CCCc2ccc3c(n2)NCCC3)C1] |
| 634 | 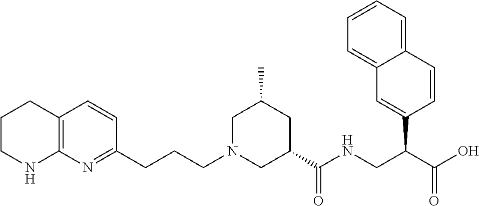 [SMILES: C[C@@H]1C[C@H](C(=O)NC[C@H](C(=O)O)c2ccc3ccccc3c2)CN(CCCc2ccc3c(n2)NCCC3)C1] |
| 635 | 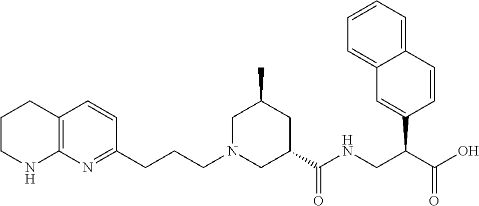 [SMILES: C[C@H]1C[C@H](C(=O)NC[C@H](C(=O)O)c2ccc3ccccc3c2)CN(CCCc2ccc3c(n2)NCCC3)C1] |
| 636 | 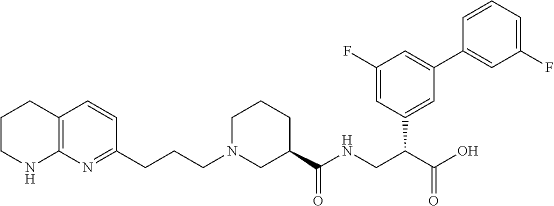 [SMILES: O=C(NC[C@@H](C(=O)O)c1cc(F)cc(-c2cccc(F)c2)c1)[C@@H]1CCCN(CCCc2ccc3c(n2)NCCC3)C1] |
| 637 | 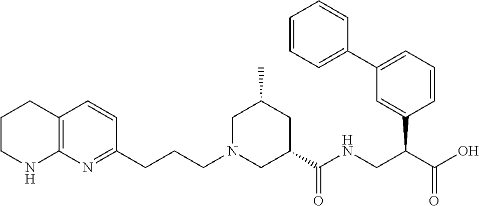 [SMILES: C[C@@H]1C[C@H](C(=O)NC[C@H](C(=O)O)c2cccc(-c3ccccc3)c2)CN(CCCc2ccc3c(n2)NCCC3)C1] |
| 638 | 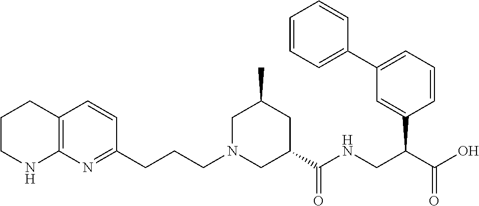 [SMILES: C[C@H]1C[C@H](C(=O)NC[C@H](C(=O)O)c2cccc(-c3ccccc3)c2)CN(CCCc2ccc3c(n2)NCCC3)C1] |
| 639 | 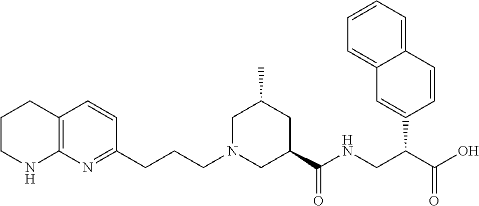 [SMILES: C[C@@H]1C[C@@H](C(=O)NC[C@@H](C(=O)O)c2ccc3ccccc3c2)CN(CCCc2ccc3c(n2)NCCC3)C1] |
| 640 | 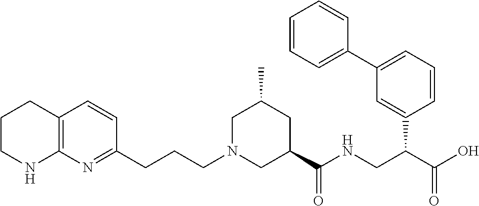 [SMILES: C[C@@H]1C[C@@H](C(=O)NC[C@@H](C(=O)O)c2cccc(-c3ccccc3)c2)CN(CCCc2ccc3c(n2)NCCC3)C1] |
| 641 | 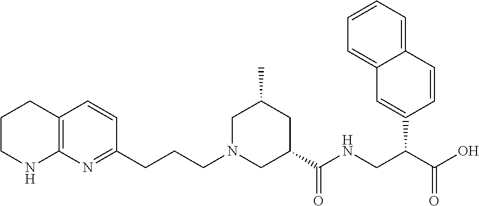 [SMILES: C[C@@H]1C[C@H](C(=O)NC[C@@H](C(=O)O)c2ccc3ccccc3c2)CN(CCCc2ccc3c(n2)NCCC3)C1] |
| 642 | 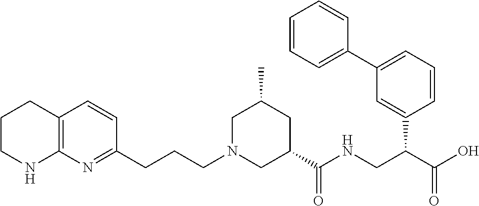 [SMILES: C[C@@H]1C[C@H](C(=O)NC[C@@H](C(=O)O)c2cccc(-c3ccccc3)c2)CN(CCCc2ccc3c(n2)NCCC3)C1] |
| 643 | 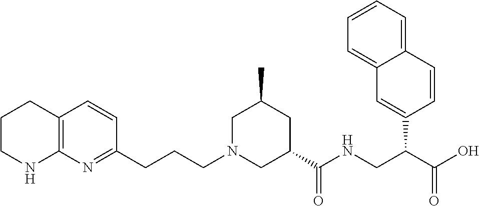 [SMILES: C[C@H]1C[C@H](C(=O)NC[C@@H](C(=O)O)c2ccc3ccccc3c2)CN(CCCc2ccc3c(n2)NCCC3)C1] |
| 644 | 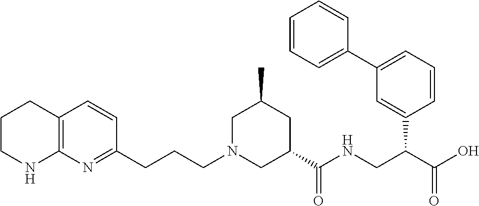 [SMILES: C[C@H]1C[C@H](C(=O)NC[C@@H](C(=O)O)c2cccc(-c3ccccc3)c2)CN(CCCc2ccc3c(n2)NCCC3)C1] |
| 645 | 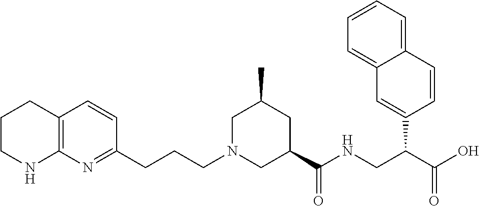 [SMILES: C[C@H]1C[C@@H](C(=O)NC[C@@H](C(=O)O)c2ccc3ccccc3c2)CN(CCCc2ccc3c(n2)NCCC3)C1] |
| 646 | 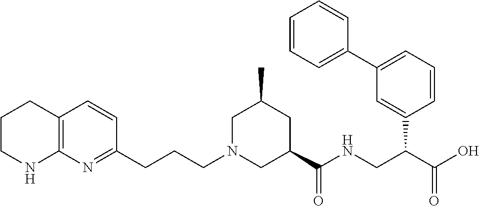 [SMILES: C[C@H]1C[C@@H](C(=O)NC[C@@H](C(=O)O)c2cccc(-c3ccccc3)c2)CN(CCCc2ccc3c(n2)NCCC3)C1] |
| 647 | 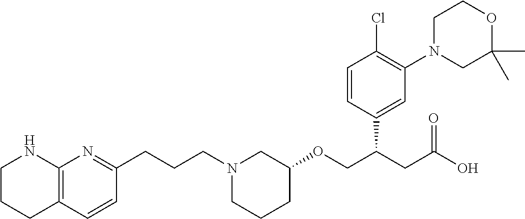 [SMILES: CC1(C)CN(c2cc([C@@H](CO[C@@H]3CCCN(CCCc4ccc5c(n4)NCCC5)C3)CC(=O)O)ccc2Cl)CCO1] |
| 648 | 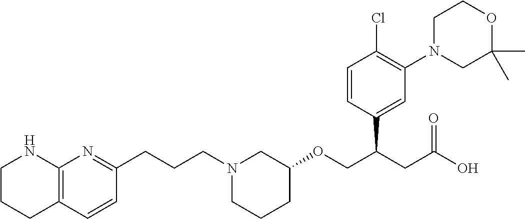 [SMILES: CC1(C)CN(c2cc([C@H](CO[C@@H]3CCCN(CCCc4ccc5c(n4)NCCC5)C3)CC(=O)O)ccc2Cl)CCO1] |
| 649 | 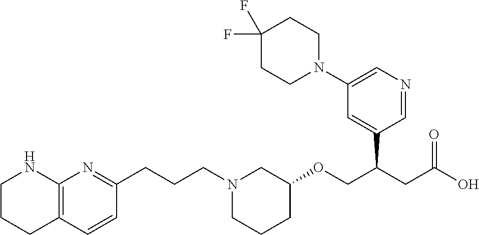 [SMILES: O=C(O)C[C@@H](CO[C@@H]1CCCN(CCCc2ccc3c(n2)NCCC3)C1)c1cncc(N2CCC(F)(F)CC2)c1] |
| 650 | 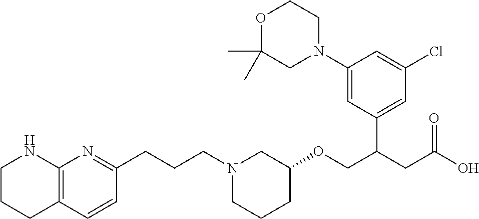 [SMILES: CC1(C)CN(c2cc(Cl)cc(C(CO[C@@H]3CCCN(CCCc4ccc5c(n4)NCCC5)C3)CC(=O)O)c2)CCO1] |
| 651 | 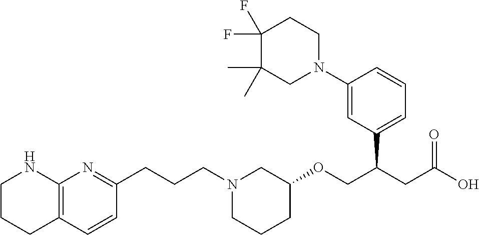 [SMILES: CC1(C)CN(c2cccc([C@H](CO[C@@H]3CCCN(CCCc4ccc5c(n4)NCCC5)C3)CC(=O)O)c2)CCC1(F)F] |
| 652 | 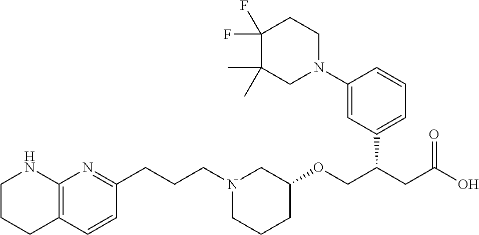 [SMILES: CC1(C)CN(c2cccc([C@@H](CO[C@@H]3CCCN(CCCc4ccc5c(n4)NCCC5)C3)CC(=O)O)c2)CCC1(F)F] |

### Methods of Synthesis

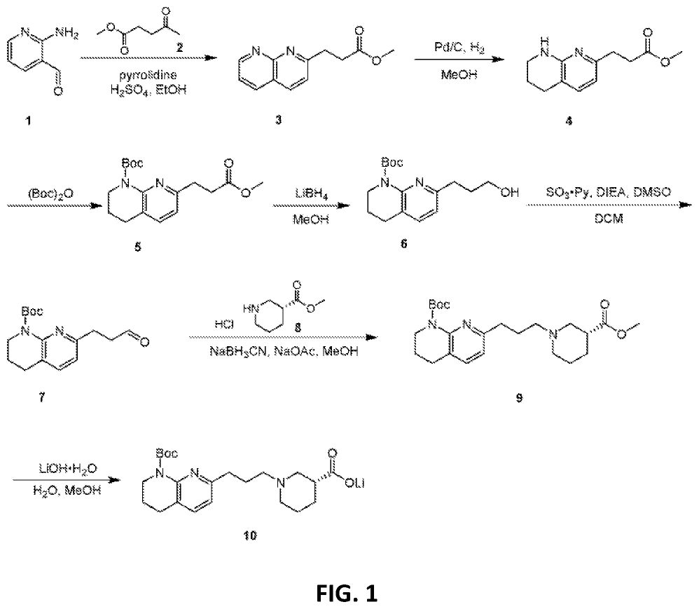

Referring now to FIG. 1, which describes the synthesis of intermediate 10, condensation of pyridyl aldehyde 1 with keto ester 2 in the presence of pyrrolidine in sulfuric acid yields the annulated ester 3, which is reduced under conventional conditions to provide the crude pyridyl ester 4. Protection of the free amine yields the Boc derivative 5, which is reduced with lithium borohydride to provide alcohol 6. Parikh-Doering oxidation of alcohol 6 yields crude aldehyde 7 which is condensed with the amine 8 under reducing conditions to provide the 3-pyridyl ester 9. Hydrolysis of ester 9 provides the crude lithium salt 10.

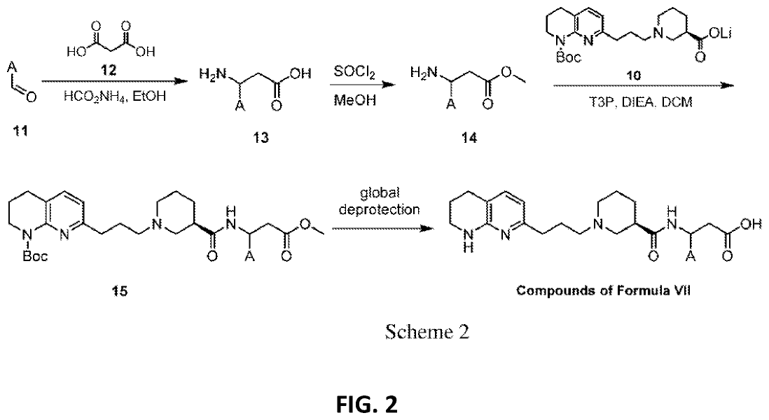

Referring now to FIG. 2, which describes synthesis of compounds of Formula (VII), generic aldehyde 11 is condensed with malonic acid 12 in the presence of ammonium formate to provide β-amino acid 13 which is then esterified to yield β-amino ester 14. Reaction of β-amino ester 14 with compound 10 provides α-amino ester amide 15 which is globally deprotected to yield compounds of Formula (VII).

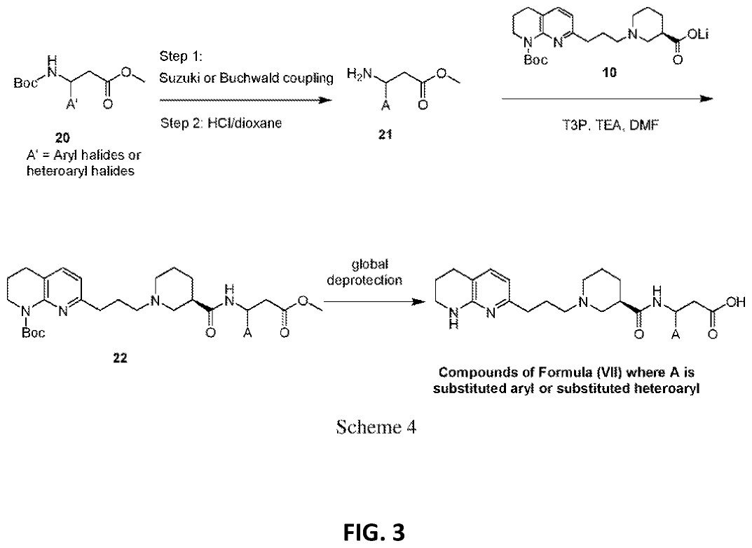

Referring now to FIG. 3 which describes another synthesis of compounds of Formula (VII), cross coupling of the aryl halide with a transition metal compound provides compound 21 after acid deprotection, which is reacted with compound 10 to give ester amide 22 which is globally deprotected to yield compounds of Formula (VII).

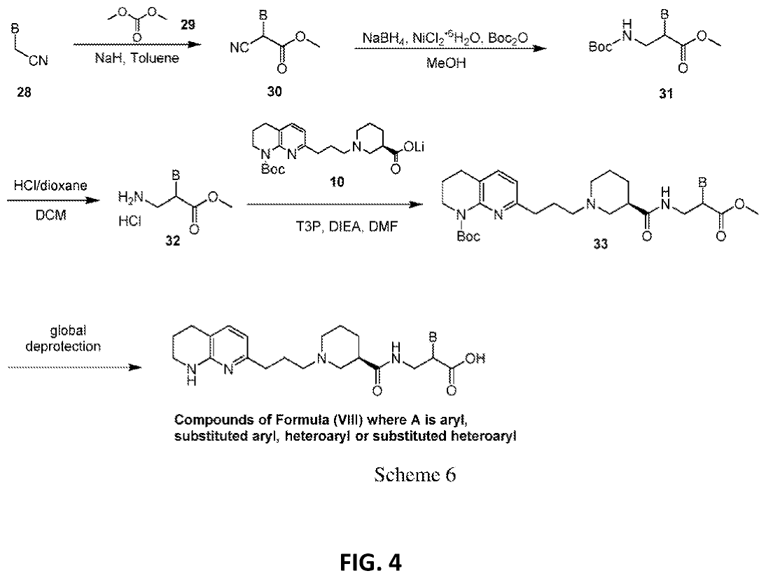

Referring now to FIG. 4 which describes synthesis of compounds of Formula (VIII), nitrile 28 is reacted with dimethyl carbonate 29 to yield ester 30. Reduction of nitrile and protection of the amine provided the β-amino ester 31, which was deprotected to yield the amine salt 32. Reaction of amine salt 32 with compound 10 gives ester amide 33, which is globally deprotected to yield compounds of Formula (VIII).

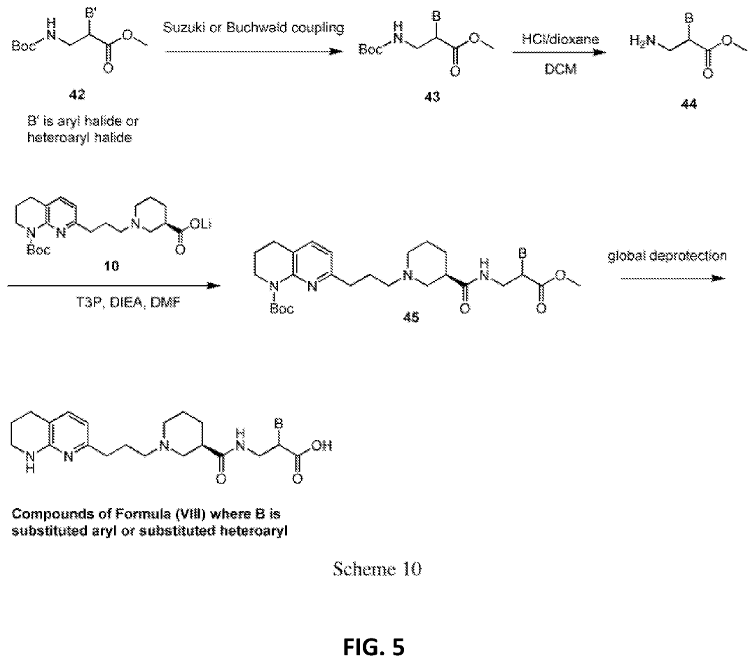

Referring now to FIG. 5 which describes another synthesis of compounds of Formula (VIII), cross coupling of aryl halide 42 with a transition metal compound provides compound 43 which is deprotected to yield compound 44. Reaction of compound 44 with compound 10 gives ester amide 45 which is globally deprotected to yield compounds of Formula (VIII).

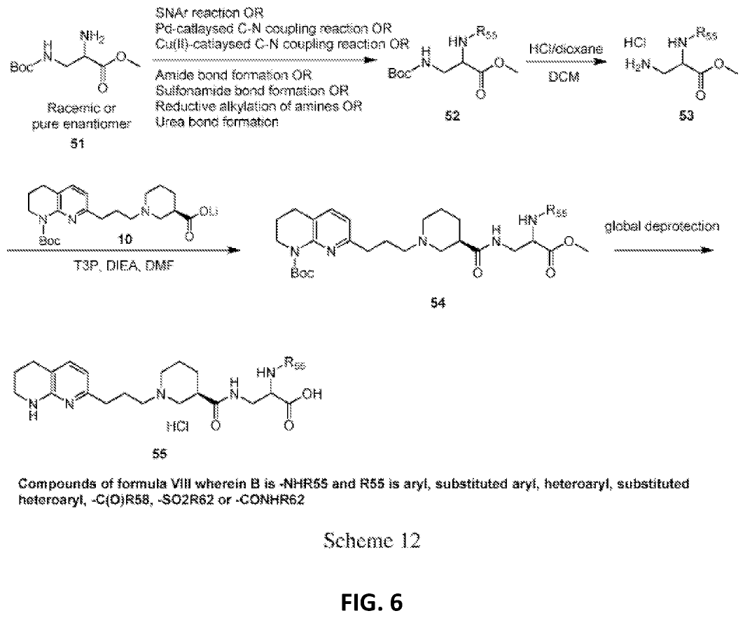

Referring now to FIG. 6 which describes the synthesis of amides and sulfonamides of Formula (VIII), reaction of free amine 51 under a variety of conditions can be used to provide amine, amide, urea or sulfonamide 52, which is deprotected to provide the amine salt 53. Reaction of amine salt 53 with compound 10 gives ester amide 45 which is globally deprotected to yield compounds of Formula (VIII).

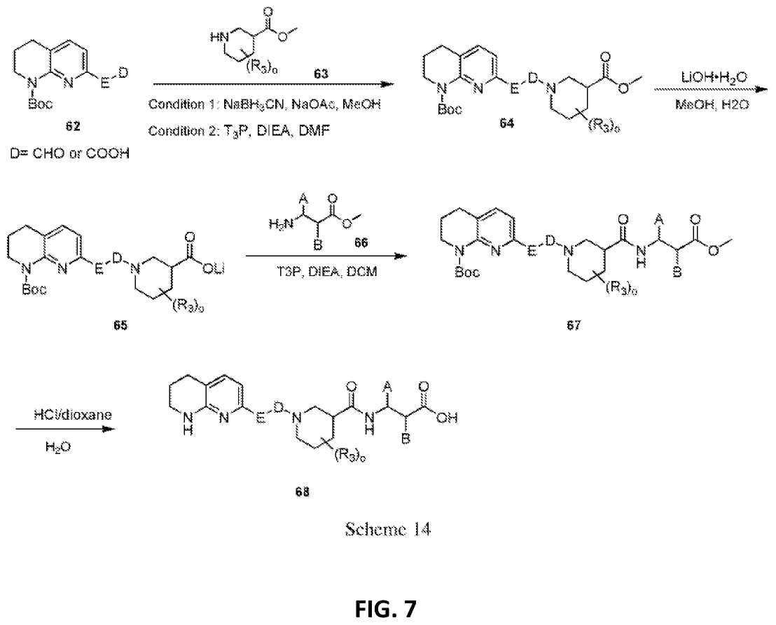

Referring now to FIG. 7, compound 62 is either acylated or reductively alkylated with substituted piperidinyl ester 63 to provide compound 64. Hydrolysis of compound 64 yielded lithium salt 65, which was then reacted with ester 66 to provide compound 67 which is globally deprotected to yield compound 68.

The skilled artisan will appreciate that many other synthetic routes can be implemented to provide compounds of Formulae (I) to (VIII) and (Ia) to (IIIa). Accordingly, the methods illustrated in FIGS. 1-7 should be considered representative rather than comprehensive.

### Methods of Use

Methods of treating, preventing, or ameliorating symptoms of medical disorders such as, for example, idiopathic pulmonary fibrosis, systemic sclerosis associated interstitial lung disease, myositis associated interstitial lung disease, systemic lupus erythematosus associated interstitial lung disease, rheumatoid arthritis, associated interstitial lung disease, diabetic nephropathy, focal segmental glomerulosclerosis, chronic kidney disease, nonalcoholic steatohepatitis, primary biliary cholangitis, primary sclerosing cholangitis, solid tumors, hematological tumors, organ transplant, Alport syndrome, interstitial lung disease, radiation-induced fibrosis, bleomycin-induced fibrosis, asbestos-induced fibrosis, flu-induced fibrosis, coagulation-induced fibrosis, vascular injury-induced fibrosis, aortic stenosis, and cardiac fibrosis in a patient with a need thereof are provided. In practicing the methods, therapeutically effective amounts of the compounds of Formula (I) and/or pharmaceutical compositions thereof are administered to the patient with the disorder or condition.

Exemplary solid tumors (e.g., sarcomas, carcinomas, and lymphomas) that may be treated with compounds of Formula (I) and pharmaceutical compositions thereof include, for example, Ewing's sarcoma, rhabdomyosarcoma, osteosarcoma, myelosarcoma, chondrosarcoma, liposarcoma, leiomyosarcoma, soft tissue sarcoma, non-small cell lung cancer, small cell lung cancer, bronchus cancer, prostate cancer, breast cancer, pancreatic cancer, gastrointestinal cancer, colon cancer, rectum cancer, colon carcinoma, colorectal adenoma, thyroid cancer, liver cancer, intrahepatic bile duct cancer, hepatocellular cancer, adrenal gland cancer, stomach cancer, gastric cancer, glioma (e.g., adult, childhood brain stem, childhood cerebral astrocytoma, childhood visual pathway and hypothalamic), glioblastoma, endometrial cancer, melanoma, kidney cancer, renal pelvis cancer, urinary bladder cancer, uterine corpus, uterine cervical cancer, vaginal cancer, ovarian cancer, multiple myeloma, esophageal cancer, brain cancer (e.g., brain stem glioma, cerebellar astrocytoma, cerebral astrocytoma/malignant glioma, ependymoma, medulloblastoma, supratentorial primitive neuroectodermal tumors, visual pathway and hypothalamic glioma), lip and oral cavity and pharynx, larynx, small intestine, melanoma, villous colon adenoma, a neoplasia, a neoplasia of epithelial character, lymphomas (e.g., AIDS-related, Burkitt's, cutaneous T-cell, Hodgkin, non-Hodgkin, and primary central nervous system), a mammary carcinoma, basal cell carcinoma, squamous cell carcinoma, actinic keratosis, tumor diseases, including solid tumors, a tumor of the neck or head, polycythemia vera, essential thrombocythemia, myelofibrosis with myeloid metaplasia, Waldenstrom's macroglobulinemia, adrenocortical carcinoma. AIDS-related cancers, childhood cerebellar astrocytoma, childhood cerebellar astrocytoma, basal cell carcinoma, extrahepatic bile duct cancer, malignant fibrous histiocytoma bone cancer, bronchial adenomas/carcinoids, carcinoid tumor, gastrointestinal carcinoid tumor, primary central nervous system, cerebellar astrocytoma, childhood cancers, ependymoma, extracranial germ cell tumor, extragonadal germ cell tumor, extrahepatic bile duct cancer, intraocular melanoma eye cancer, retinoblastoma eye cancer, gallbladder cancer, gastrointestinal carcinoid tumor, germ cell tumors (e.g., extracranial, extragonadal, and ovarian), gestational trophoblastic tumor, hepatocellular cancer, hypopharyngeal cancer, hypothalamic and visual pathway glioma, islet cell carcinoma (endocrine pancreas), laryngeal cancer, malignant fibroushistiocytoma of bone/osteosarcoma, meduloblastoma, mesothelioma, metastatic squamous neck cancer with occult primary, multiple endocrine neoplasia syndrome, multiple myeloma/plasma cell neoplasm, mycosis fungoides, nasal cavity and paranasal sinus cancer, nasopharyngeal cancer, neuroblastoma, oral cancer, oropharyngeal cancer, ovarian epithelial cancer, ovarian germ cell tumor, ovarian low malignant potential tumor, islet cell pancreatic cancer, parathyroid cancer, pheochromocytoma, pineoblastoma, pituitary tumor, pleuropulmonary blastoma, ureter transitional cell cancer, retinoblastoma, rhabdomyosarcoma, salivary gland cancer, Sezary syndrome, non-melanoma skin cancer, Merkel cell carcinoma, squamous cell carcinoma, testicular cancer, thymoma, gestational trophoblastic tumor, and Wilms' tumor.

Exemplary hematological tumors (e.g., sarcomas, carcinomas, and lymphomas) that may be treated with compounds of Formula (I) and pharmaceutical compositions thereof include, for example, acute lymphocytic leukemia, acute myelogenous leukemia, chronic lymphocytic leukemia, chronic myelogenous leukemia. Hodgkin lymphoma, non-Hodgkin lymphoma, and multiple myeloma.

Also provided herein are methods for inhibiting αVβ6 integrin in a patient. In practicing the methods, therapeutically effective amounts of the compounds of Formula (I) or pharmaceutical compositions thereof are administered to the patient with the disorder or condition.

Also provided herein are methods for inhibiting (αVβ1 integrin in a patient. In practicing the methods, therapeutically effective amounts of the compounds of Formula (I) or pharmaceutical compositions thereof are administered to the patient with the disorder or condition.

Also provided herein are methods for inhibiting TGFβ activation in a cell. In practicing the methods, effective amounts of the compounds or Formula (I) or pharmaceutical compositions thereof are administered to the patient with the disorder or condition.

### Compositions and Methods of Administration

The compositions provided herein contain therapeutically effective amounts of one or more of the compounds provided herein that are useful in the prevention, treatment, or amelioration of one or more of the symptoms of diseases or disorders described herein and a vehicle. Vehicles suitable for administration of the compounds provided herein include any such carriers known to those skilled in the art to be suitable for the particular mode of administration. In addition, the compounds may be formulated as the sole active ingredient in the composition or may be combined with other active ingredients.

The compositions contain one or more compounds provided herein. The compounds are, in some embodiments, formulated into suitable preparations such as solutions, suspensions, tablets, dispersible tablets, pills, capsules, powders, sustained release formulations or elixirs, for oral administration or in sterile solutions or suspensions for parenteral administration, as well as topical administration, transdermal administration and oral inhalation via nebulizers, pressurized metered dose inhalers and dry powder inhalers. In some embodiments, the compounds described above are formulated into compositions using techniques and procedures well known in the art (see, e.g., Ansel. Introduction to Pharmaceutical Dosage Forms, Seventh Edition (1999)).

In the compositions, effective concentrations of one or more compounds or derivatives thereof is (are) mixed with a suitable vehicle. The compounds may be derivatized as the corresponding salts, esters, enol ethers or esters, acetals, ketals, orthoesters, hemiacetals, hemiketals, acids, bases, solvates, ion-pairs, hydrates or prodrugs prior to formulation, as described above. The concentrations of the compounds in the compositions are effective for delivery of an amount, upon administration that treats, leads to prevention, or amelioration of one or more of the symptoms of diseases or disorders described herein. In some embodiments, the compositions are formulated for single dosage administration. To formulate a composition, the weight fraction of a compound is dissolved, suspended, dispersed or otherwise mixed in a selected vehicle at an effective concentration such that the treated condition is relieved, prevented, or one or more symptoms are ameliorated.

The active compound is included in the vehicle in an amount sufficient to exert a therapeutically useful effect in the absence of undesirable side effects on the patient treated. The therapeutically effective concentration may be predicted empirically by testing the compounds in in vitro and in vivo systems well known to those of skill in the art and then extrapolated therefrom for dosages for humans. Human doses are then typically fine-tuned in clinical trials and titrated to response.

The concentration of active compound in the composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of diseases or disorders as described herein.

In instances in which the compounds exhibit insufficient solubility, methods for solubilizing compounds may be used such as use of liposomes, prodrugs, complexation/chelation, nanoparticles, or emulsions or tertiary templating. Such methods are known to those of skill in this art, and include, but are not limited to, using co-solvents, such as dimethylsulfoxide (DMSO), using surfactants or surface modifiers, such as TWEEN®, complexing agents such as cyclodextrin or dissolution by enhanced ionization (i.e. dissolving in aqueous sodium bicarbonate). Derivatives of the compounds, such as prodrugs of the compounds may also be used in formulating effective compositions.

Upon mixing or addition of the compound(s), the resulting mixture may be a solution, suspension, emulsion or the like. The form of the resulting mixture depends upon a number of factors, including the intended mode of administration and the solubility of the compound in the selected vehicle. The effective concentration is sufficient for ameliorating the symptoms of the disease, disorder or condition treated and may be empirically determined.

The compositions are provided for administration to humans and animals in indication appropriate dosage forms, such as dry powder inhalers (DPIs), pressurized metered dose inhalers (pMDIs), nebulizers, tablets, capsules, pills, sublingual tapes/bioerodible strips, tablets or capsules, powders, granules, lozenges, lotions, salves, suppositories, fast melts, transdermal patches or other transdermal application devices/preparations, sterile parenteral solutions or suspensions, and oral solutions or suspensions, and oil-water emulsions containing suitable quantities of the compounds or derivatives thereof. The therapeutically active compounds and derivatives thereof are, in some embodiments, formulated and administered in unit-dosage forms or multiple-dosage forms. Unit-dose forms as used herein refer to physically discrete units suitable for human and animal subjects and packaged individually as is known in the art. Each unit-dose contains a predetermined quantity of the therapeutically active compound sufficient to produce the desired therapeutic effect, in association with the required vehicle. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid compositions can, for example, be prepared by dissolving, dispersing, or otherwise mixing an active compound as defined above and optional adjuvants in a vehicle, such as, for example, water, saline, aqueous dextrose, glycerol, glycols, ethanol, and the like, to thereby form a solution or suspension, colloidal dispersion, emulsion or liposomal formulation. If desired, the composition to be administered may also contain minor amounts of nontoxic auxiliary substances such as wetting agents, emulsifying agents, solubilizing agents, pH buffering agents and the like, for example, acetate, sodium citrate, cyclodextrin derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975 or later editions thereof.
Dosage forms or compositions containing active ingredient in the range of 0.005% to 100% with the balance made up from vehicle or carrier may be prepared. Methods for preparation of these compositions are known to those skilled in the art. The contemplated compositions may contain 0.001%-100% active ingredient, in one embodiment 0.1-95%, in another embodiment 0.4-10%.

In certain embodiments, the compositions are lactose-free compositions containing excipients that are well known in the art and are listed, for example, in the U.S. Pharmacopeia (USP) 25-NF20 (2002). In general, lactose-free compositions contain active ingredients, a binder/filler, and a lubricant in compatible amounts. Particular lactose-free dosage forms contain active ingredients, microcrystalline cellulose, pre-gelatinized starch, and magnesium stearate.

Further provided are anhydrous compositions and dosage forms comprising active ingredients, since water can facilitate the degradation of some compounds. For example, the addition of water (e.g., 5%) is widely accepted as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, NY, 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous compositions and dosage forms provided herein can be prepared using anhydrous or low moisture containing ingredients and low moisture or low humidity conditions.

An anhydrous composition should be prepared and stored such that its anhydrous nature is maintained. Accordingly, anhydrous compositions are generally packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Oral dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms such as for example, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an enteric coating; a film coating agent and modified release agent. Examples of binders include microcrystalline cellulose, methyl paraben, polyalkyleneoxides, gum tragacanth, glucose solution, acacia mucilage, gelatin solution, molasses, poly vinylpyrrolidine, povidone, crospovidones, sucrose and starch and starch derivatives. Lubricants include talc, starch, magnesium/calcium stearate, lycopodium and stearic acid. Diluents include, for example, lactose, sucrose, trehalose, lysine, leucine, lecithin, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate and advanced coloring or anti-forgery color/opalescent additives known to those skilled in the art. Sweetening agents include sucrose, lactose, mannitol and artificial sweetening agents such as saccharin and any number of spray dried flavors. Flavoring agents include natural flavors extracted from plants such as fruits and synthetic blends of compounds which produce a pleasant sensation or mask unpleasant taste, such as, but not limited to peppermint and methyl salicylate. Wetting agents include propylene glycol monostearate, sorbitan monooleate, diethylene glycol monolaurate and polyoxyethylene lauryl ether. Enteric-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate. Modified release agents include polymers such as the Eudragit® series and cellulose esters.

The compound, or derivative thereof, can be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids. H₂ blockers, and diuretics. The active ingredient is a compound or derivative thereof as described herein. Higher concentrations, up to about 98% by weight of the active ingredient may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Vehicles used in elixirs include solvents. Syrups are concentrated aqueous solutions of a sugar, for example, sucrose, and may contain a preservative. An emulsion is a two-phase system in which one liquid is dispersed in the form of small globules throughout another liquid. Carriers used in emulsions are non-aqueous liquids, emulsifying agents and preservatives. Suspensions use suspending agents and preservatives. Acceptable substances used in non-effervescent granules, to be reconstituted into a liquid oral dosage form, include diluents, sweeteners and wetting agents. Acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxy ethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example, propylene carbonate, vegetable oils or triglycerides, is in some embodiments encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a liquid vehicle, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or polyalkylene glycol, including, but not limited to, 1,2-dimethoxyethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including an acetal. Alcohols used in these formulations are any water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

Parenteral administration, in some embodiments characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see. e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Vehicles used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose and polyvinylpyrrolidone. Emulsifying agents include Polysorbate 80 (Tween® 80). A sequestering or chelating agent of metal ions includes EDTA. Carriers also include ethyl alcohol, polyethylene glycol and propylene glycol for water miscible vehicles; and sodium hydroxide, hydrochloric acid, citric acid or lactic acid for pH adjustment.

The concentration of compound is adjusted so that an injection provides an effective amount to produce the desired pharmacological effect. The exact dose depends on the age, weight, body surface area and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In some embodiments, a therapeutically effective dosage is formulated to contain a concentration of at least about 0.01% w/w up to about 90% w/w or more, in certain embodiments more than 0.1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981, 6,376,461; 6,419,961; 6,589,548; 6,613,358; 6,699,500 and 6,740,634. Such dosage forms can be used to provide slow or controlled-release of one or more active ingredients using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain embodiments, the agent may be administered using intravenous infusion, an implantable osmotic pump, a transdermal patch, liposomes, or other modes of administration. In some embodiments, a pump may be used (see. Sefton, CRC Crit. Ref Biomed. Eng. 14:201 (1987); Buchwald et al., Surgery 88:507 (1980); Saudek et al., N. Engl. J Med. 321:574 (1989)). In other embodiments, polymeric materials can be used. In other embodiments, a controlled release system can be placed in proximity of the therapeutic target, i.e., thus requiring only a fraction of the systemic dose (see. e.g., Goodson, Medical Applications of Controlled Release, vol. 2, pp. 115-138 (1984)). In some embodiments, a controlled release device is introduced into a subject in proximity of the site of inappropriate immune activation or a tumor. Other controlled release systems are discussed in the review by Langer (Science 249:1527-1533 (1990)). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, poly vinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a derivative thereof, in a suitable solvent. The solvent may contain an excipient which improves the stability or other pharmacological component of the powder or reconstituted solution, prepared from the powder. Excipients that may be used include, but are not limited to, an antioxidant, a buffer and a bulking agent. In some embodiments, the excipient is selected from dextrose, sorbitol, fructose, corn syrup, xylitol, glycerin, glucose, sucrose and other suitable agent. The solvent may contain a buffer, such as citrate, sodium or potassium phosphate or other such buffer known to those of skill in the art at, at about neutral pH. Subsequent sterile filtration of the solution followed by lyophilization under standard conditions known to those of skill in the art provides the desired formulation. In some embodiments, the resulting solution will be apportioned into vials for lyophilization. Each vial will contain a single dosage or multiple dosages of the compound. The lyophilized powder can be stored under appropriate conditions, such as at about 4° C., to room temperature.

Reconstitution of this lyophilized powder with water for injection provides a formulation for use in parenteral administration. For reconstitution, the lyophilized powder is added to sterile water or another suitable carrier. The precise amount depends upon the selected compound. Such amount can be empirically determined.

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, in some embodiments, have mass median geometric diameters of less than 5 microns, in other embodiments less than 10 microns.

Oral inhalation formulations of the compounds or derivatives suitable for inhalation include metered dose inhalers, dry powder inhalers and liquid preparations for administration from a nebulizer or metered dose liquid dispensing system. For both metered dose inhalers and dry powder inhalers, a crystalline form of the compounds or derivatives is the preferred physical form of the drug to confer longer product stability.

In addition to particle size reduction methods known to those skilled in the art, crystalline particles of the compounds or derivatives can be generated using supercritical fluid processing which offers significant advantages in the production of such particles for inhalation delivery by producing respirable particles of the desired size in a single step. (e.g., International Publication No. WO2005/025506). A controlled particle size for the microcrystals can be selected to ensure that a significant fraction of the compounds or derivatives is deposited in the lung. In some embodiments, these particles have a mass median aerodynamic diameter of about 0.1 to about 10 microns, in other embodiments, about 1 to about 5 microns and still other embodiments, about 1.2 to about 3 microns.

Inert and non-flammable HFA propellants are selected from HFA 134a (1,1,1,2-tetrafluoroethane) and HFA 227e (1,1,1,2,3,3,3-heptafluoropropane) and provided either alone or as a ratio to match the density of crystal particles of the compounds or derivatives. A ratio is also selected to ensure that the product suspension avoids detrimental sedimentation or cream (which can precipitate irreversible agglomeration) and instead promote a loosely flocculated system, which is easily dispersed when shaken. Loosely fluctuated systems are well regarded to provide optimal stability for pMDI canisters. As a result of the formulation's properties, the formulation contained no ethanol and no surfactants/stabilizing agents.

The compounds may be formulated for local or topical application, such as for topical application to the skin and mucous membranes, such as in the eye, in the form of gels, creams, and lotions and for application to the eye or for intracistemal or intraspinal application. Topical administration is contemplated for transdermal delivery and also for administration to the eyes or mucosa, or for inhalation therapies. Nasal solutions of the active compound alone or in combination with other excipients can also be administered.

For nasal administration, the preparation may contain an esterified phosphonate compound dissolved or suspended in a liquid carrier, in particular, an aqueous carrier, for aerosol application. The carrier may contain solubilizing or suspending agents such as propylene glycol, surfactants, absorption enhancers such as lecithin or cyclodextrin, or preservatives. Solutions, particularly those intended for ophthalmic use, may be formulated as 0.01%-10% isotonic solutions, pH about 5-7.4, with appropriate salts.

Other routes of administration, such as transdermal patches, including iontophoretic and electrophoretic devices, and rectal administration, are also contemplated herein.

Transdermal patches, including iontophoretic and electrophoretic devices, are well known to those of skill in the art. For example, such patches are disclosed in U.S. Pat. Nos. 6,267,983, 6,261,595, 6,256,533, 6,167,301, 6,024,975, 6,010,715, 5,985,317, 5,983,134, 5,948,433 and 5,860,957.

For example, dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in one embodiment, is about 2 to 3 gm. Tablets and capsules for rectal administration are manufactured using the same substance and by the same methods as for formulations for oral administration.

The compounds provided herein, or derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In some embodiments, liposomal suspensions, including tissue-targeted liposomes, such as tumor-targeted liposomes, may also be suitable as carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down phosphatidyl choline and phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The compounds or derivatives thereof may be packaged as articles of manufacture containing packaging material, a compound or derivative thereof provided herein, which is effective for treatment, prevention or amelioration of one or more symptoms of the diseases or disorders, supra, within the packaging material, and a label that indicates that the compound or composition or derivative thereof, is used for the treatment, prevention or amelioration of one or more symptoms of the diseases or disorders, supra.

The articles of manufacture provided herein contain packaging materials. Packaging materials for use in packaging products are well known to those of skill in the art. See, e.g., U.S. Pat. Nos. 5,323,907, 5,052,558 and 5,033,252. Examples of packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease or disorder described herein.

### Dosages

For use to treat or prevent disease, the compounds described herein, or pharmaceutical compositions thereof, are administered or applied in a therapeutically effective amount. In human therapeutics, the physician will determine the dosage regimen that is most appropriate according to a preventive or curative treatment and according to the age, weight, stage of the disease and other factors specific to the subject to be treated. The amount of active ingredient in the formulations provided herein, which will be effective in the prevention or treatment of an infectious disease will vary with the nature and severity of the disease or condition, and the route by which the active ingredient is administered. The frequency and dosage will also vary according to factors specific for each subject depending on the specific therapy (e.g., therapeutic or prophylactic agents) administered, the severity of the infection, the route of administration, as well as age, body, weight, response, and the past medical history of the subject.

Exemplary doses of a formulation include milligram or microgram amounts of the active compound per kilogram of subject (e.g., from about 1 microgram per kilogram to about 50 milligrams per kilogram, from about 10 micrograms per kilogram to about 30 milligrams per kilogram, from about 100 micrograms per kilogram to about 10 milligrams per kilogram, or from about 100 micrograms per kilogram to about 5 milligrams per kilogram).

In some embodiments, a therapeutically effective dosage should produce a serum concentration of active ingredient of from about 0.001 ng/ml to about 50-200 μg/ml. The compositions, in other embodiments, should provide a dosage of from about 0.0001 mg to about 70 mg of compound per kilogram of body weight per day. Dosage unit forms are prepared to provide from about 0.01 mg, 0.1 mg or 1 mg to about 500 mg, 1000 mg or 5000 mg, and in some embodiments from about 10 mg to about 500 mg of the active ingredient or a combination of essential ingredients per dosage unit form.

The active ingredient may be administered at once or may be divided into a number of smaller doses to be administered at intervals of time. It is understood that the precise dosage and duration of treatment is a function of the disease being treated and may be determined empirically using known testing protocols or by extrapolation from in vivo or in vitro test data or subsequent clinical testing. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions and that the concentration ranges set forth herein are exemplary only and are not intended to limit the scope or practice of the claimed compositions.

It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with subject response.

For systemic administration, a therapeutically effective dose can be estimated initially from in vitro assays. For example, a dose can be formulated in animal models to achieve a circulating concentration range that includes the IC₅₀ as determined in cell culture (i.e., the concentration of test compound that is lethal to 50% of a cell culture), or the IC₁₀₀ as determined in cell culture (i.e., the concentration of compound that is lethal to 100% of a cell culture). Such information can be used to more accurately determine useful doses in humans.

Initial dosages can also be estimated from in vivo data (e.g., animal models) using techniques that are well known in the art. One of ordinary skill in the art can readily optimize administration to humans based on animal data.

Alternatively, initial dosages can be determined from the dosages administered of known agents by comparing the IC₅₀, MIC and/or I₁₀₀ of the specific compound disclosed herein with that of a known agent and adjusting the initial dosages accordingly. The optimal dosage may be obtained from these initial values by routine optimization

In cases of local administration or selective uptake, the effective local concentration compound used may not be related to plasma concentration. One of skill in the art will be able to optimize therapeutically effective local dosages without undue experimentation.

Ideally, a therapeutically effective dose of the compounds described herein will provide therapeutic benefit without causing substantial toxicity. Toxicity of compounds can be determined using standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Compounds which exhibit high therapeutic indices are preferred. The data obtained from these cell culture assays, and animal studies can be used in formulating a dosage range that is not toxic for use in subjects. The dosage of the compounds described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition (See, e.g., Fingl et al., 1975, in The Pharmacological Basis of Therapeutics, Ch. 1, p. 1).

The therapy may be repeated intermittently. In certain embodiments, administration of the same formulation provided herein may be repeated and the administrations may be separated by at least 1 day, 2 days, 3 days, 5 days, 10 days, 15 days, 30 days, 45 days, 2 months, 75 days, 3 months, or 6 months.

### Combination Therapy

The compounds of Formula (I) and pharmaceutical compositions thereof disclosed herein may also be used in combination with one or more other active ingredients. In certain embodiments, the compounds of Formula (I) and pharmaceutical compositions thereof may be administered in combination, or sequentially, with another therapeutic agent. Such other therapeutic agents include those known for treatment, prevention, or amelioration of one or more symptoms associated with idiopathic pulmonary fibrosis, interstitial lung disease, systemic lupus erythematosus associated interstitial lung disease, rheumatoid arthritis, diabetic nephropathy, focal segmental glomerulosclerosis, chronic kidney disease, nonalcoholic steatohepatitis, primary biliary cholangitis, primary sclerosing cholangitis, solid tumors, hematological tumors, organ transplant, Alport syndrome, interstitial lung disease, radiation-induced fibrosis, bleomycin-induced fibrosis, asbestos-induced fibrosis, flu-induced fibrosis, coagulation-induced fibrosis, vascular injury-induced fibrosis, aortic stenosis, and cardiac fibrosis.

Exemplary therapeutic agents which may be used with the compounds of Formula (I) and pharmaceutical compositions thereof include, but are not limited to, components and fragments of bee venom, pollen, milk, peanut, CpG motifs, collagen, other components of extracellular matrix, anti-histamines (e.g., cetirizine, loratidine, acrivastine, fexofenidine, chlorphenamine, etc.), corticosteroids (e.g., fluticasone propionate, fluticasone futroate, beclomethasone dipropionate, budesonide, ciclesonide, mometasone furoate, triamcinolone, flunisolide, prednisolone, hydrocortisone, etc.), NSAIDs (e.g., aspirin, ibuprofen, naproxen, etc.), leukotriene modulators (e.g., montelukast, zafirlukast, pranlukast, etc.), iNOS inhibitors, tryptase inhibitors, p38 inhibitors (e.g., losmapimod, dilmapimod, etc.), elastase inhibitors, beta2 agonists. DP1 antagonists, DP2 antagonists, pl 3K delta inhibitors, lysophosphatidic inhibitors or 5-lipoxygenase activating protein inhibitors (e.g., sodium 3-(3-(tert-butylthio)-1-(4-(6-ethoxypyridin-3-yl)benzyl)-5-((5-methylpyridin-2-yl) methoxy)-1H-indol-2-yl)-2,2-dimethylpropanoate, etc.), adenosine agonists (e.g., adenosine, regadenoson, etc.), chemokine antagonists (e.g., CCR3 antagonists, CCR4 antagonists, etc.), mediator release inhibitors, DMARDS (e.g., methotrexate, leflunomide, azathioprine, etc.), biopharmaceutical therapies (e.g., anti-lgE, anti-TNF, anti-interleukins (e.g., anti-IL-1, anti-IL-6, anti-IL-12, anti-I L-17, anti-I L-18, etc.), receptor therapies (e.g., etanercept), interferon, cytokines, chemokines, cytokine and chemokine receptor modulators, cytokine agonists or antagonists, TLR agonists, inhibitors of TGF synthesis (e.g., pirfenidone), tyrosine kinase inhibitors targeting the vascular endothelial growth factor (VEGF), platelet-derived growth factor (PDGF), fibroblast growth factor (FGF) receptor kinases, imatinib mesylate (i.e., Gleevec), endothelin receptor antagonists (e.g., ambrisentan or macitentan), antioxidants (e.g., N-acetylcysteine (NAC), broad-spectrum antibiotics (e.g., cotrimoxazole, tetracyclines, etc.), phosphodiesterase 5 inhibitors (e.g., sildenafil), anti-αvβχ antibodies and anti-αvββ monoclonal antibodies, bronchodilators (e.g., salbutamol), long-acting 2-agonists (e.g., salmeterol, formoterol, vilanterol, etc.), short-acting muscarinic antagonists (e.g., ipratropium bromide), long-acting muscarinic antagonists (e.g., tiotropium, umeclidinium), Lucentis® and Avastin®.

It should be understood that any suitable combination of the compounds and compositions provided herein with one or more of the above therapeutic agents and optionally one or more further pharmacologically active substances are considered to be within the scope of the present disclosure. In some embodiments, the compounds and compositions provided herein are administered prior to or subsequent to the one or more additional active ingredients.

Finally, it should be noted that there are alternative ways of implementing the present invention. Accordingly, the present embodiments are to be considered as illustrative and not restrictive, and the invention is not to be limited to the details given herein, but may be modified within the scope and equivalents of the appended claims. All publications and patents cited herein are incorporated by reference in their entirety.

The following examples are provided for illustrative purposes only and are not intended to limit the scope of the invention.

### Examples

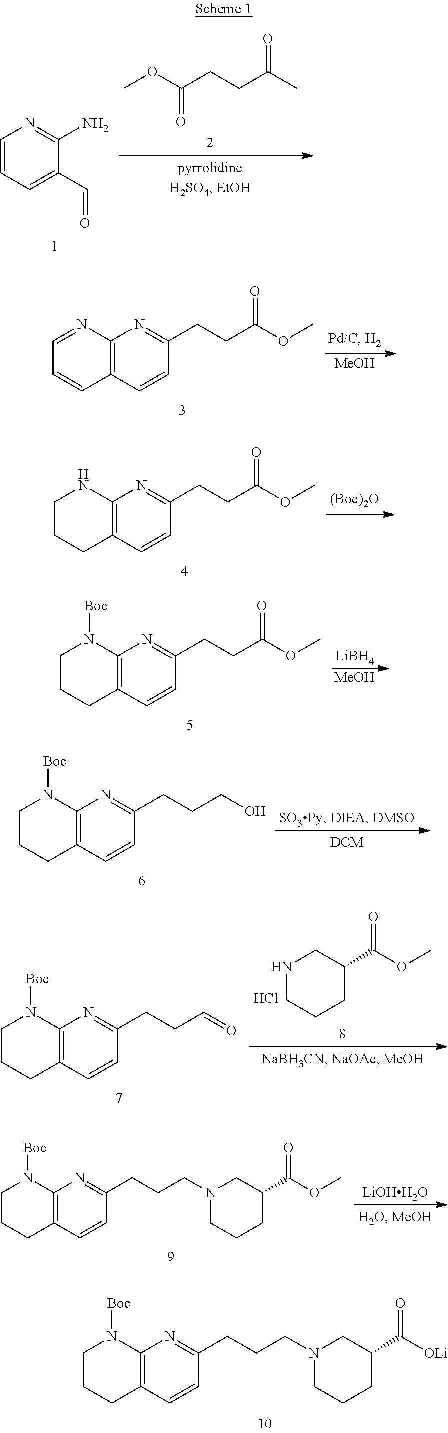

### FIG. 1

FIG. 1, Scheme 1, illustrates the synthesis of key intermediate compound 10.

### Preparation of Compound 3

To a solution of compound 1 (60.0 g, 491 mmol, 1.00 eq) and compound 2 (70.3 g, 540 mmol, 66.9 mL, 1.10 eq) in EtOH (500 mL) was added H₂SO₄ (2.36 g, 24.0 mmol, 1.28 mL, 0.0490 eq) and pyrrolidine (38.4 g, 540 mmol, 45.1 mL, 1.10 eq). The mixture was stirred at 25° C. for 12 hrs. LC-MS detected the desired mass. The reaction mixture was concentrated to give a residue which was triturated with EtOAc (80.0 mL) at 25° C. for 30 min. Compound 3 (52.0 g, 231 mmol, 47.0% yield, 96.1% purity) was obtained as a light-yellow solid. LC-MS (M+H)⁺: 217.2; ¹H NMR (400 MHz, CDCl₃): δ 9.04 (dd, J₁=4.0 Hz, J₂=1.6 Hz, 1H), 8.13 (dd, J₁=8.0 Hz, J₂=2.0 Hz, 1H), 8.07 (d, J=8.0 Hz, 1H), 7.45-7.37 (m, 2H), 3.64 (s, 3H), 3.33 (t, J=7.2 Hz, 2H), 3.04 (t, J=7.6 Hz, 2H).

### Preparation of Compound 4

To a solution of compound 3 (52.0 g, 240 mmol, 1.00 eq) in MeOH (500 mL) was added Pd/C (7.60 g, 2.03 mL, 10.0% purity) under N₂ atmosphere. The suspension was degassed and purged with H₂ for 3 times and stirred under H₂ (50 Psi) at 25° C. for 12 hrs. LC-MS detected one main peak with the desired mass. The reaction mixture was filtered and concentrated give compound 4 (52.2 g, crude) as a yellow oil. LC-MS (M+H)⁺: 221.3; ¹H NMR (400 MHz, CDCl₃): δ 7.01 (d, J=7.6 Hz, 1H), 6.30 (d, J=7.2 Hz, 1H), 3.62 (s, 3H), 3.40-3.25 (m, 2H), 2.90-2.71 (m, 2H), 2.70-2.55 (m, 4H), 1.95-1.70 (m, 2H).

### Preparation of Compound 5

A mixture of compound 4 (47.0 g, 213 mmol, 1.00 eq) and (Boc)₂O (139 g, 640 mmol, 147 mL, 3.00 eq) was stirred at 75° C. for 16 hrs. The reaction mixture was concentrated to give a residue which was purified by column chromatography (SiO₂, Petroleum ether: EtOAc=1:0 to 0:1, Petroleum ether: EtOAc=1:1, R_{f}=0.50) to provide compound 5 (35.0 g, 92.9 mmol, 43.5% yield, 85.1% purity) as a yellow solid. ¹H NMR (400 MHz, CDCl₃): δ 7.29 (d, J=7.6 Hz, 1H), 6.84 (d, J=7.2 Hz, 1H), 3.75 (t, J=5.6 Hz, 2H), 3.67 (s, 3H), 3.03 (t, J=7.2 Hz, 2H), 2.81 (t, J=7.6 Hz, 2H), 2.72 (t, J=6.8 Hz, 2H), 1.96-1.86 (m, 2H), 1.52 (s, 9H): LC-MS: (M-55)⁺: 265.2.

### Preparation of Compound 6

To a solution of compound 5 (30.0 g, 93.6 mmol, 1.00 eq) in THF (300 mL) was added LiBH₄ (4.0) M, 30.4 mL, 1.30 eq) at 0° C. The mixture was stirred at 25° C. for 8 hrs. LC-MS showed compound 5 was completely consumed and detected one main peak with the desired mass. The reaction mixture was quenched by addition of sat·aq NH₄Cl (100 mL) at 0° C., and then extracted with EtOAc (300 mL*3). The combined organic extracts were washed with brine (200 mL), dried over Na₂SO₄, filtered and concentrated to give a residue which was purified by column chromatography (SiO₂, Petroleum ether: EtOAc=1:0 to 1:2, Petroleum ether: EtOAc=1:1, R_{f}=0.25) to provide compound 6 (23.0 g, 78.6 mmol, 84.0% yield) as a yellow oil. LC-MS (M-55)⁺: 237.3; ¹H NMR (400 MHz, CDCl₃): δ 7.31 (d, J=7.6 Hz, 1H), 6.83 (d, J=7.6 Hz, 1H), 3.78-3.73 (m, 2H), 3.72-3.65 (m, 2H), 2.90 (t, J=6.8 Hz, 2H), 2.72 (t, J=6.8 Hz, 2H), 1.98-1.86 (m, 4H), 1.53 (s, 9H).

### Preparation of Compound 7

To a solution of compound 6 (5.00 g, 17.1 mmol, 1.00 eq) in DCM (50.0 mL) was added DMSO (4.01 g, 51.3 mmol, 4.01 mL, 3.00 eq), SO₃·PV (8.17 g, 51.3 mmol, 3.00 eq) and DIEA (6.63 g, 51.3 mmol, 8.94 mL, 3.00 eq) and the mixture stirred at 25° C. for 2 hrs. LC-MS showed compound 6 was completely consumed. The reaction mixture was washed with saturated citric acid solution (30.0 mL*3), dried over Na₂SO₄, filtered and concentrated under reduced pressure to give compound 7 (6.00 g, crude) as a brown oil. LC-MS (M-55)⁺: 235.2; ¹H NMR (400 MHz, DMSO-d₆): δ 9.76 (d, J=1.2 Hz, 1H). 7.41 (d, J=7.2 Hz, 1H), 6.93 (d, J=7.6 Hz, 1H), 3.62 (t, J=6.0 Hz, 2H), 2.97-2.87 (m, 2H), 2.85-2.77 (m, 2H), 2.68 (t, J=6.4 Hz, 2H), 1.83-1.76 (m, 2H), 1.43 (s, 9H).

### Preparation of Compound 9

To a solution of compound 7 (4.00 g, 13.7 mmol, 1.00 eq) in MeOH (40.0 mL) was added AcONa (1.47 g, 17.9 mmol, 1.30 eq), NaBH₃CN (1.73 g, 27.5 mmol, 2.00 eq) and compound 8 (2.97 g, 16.5 mmol, 1.20 eq, HCl). The mixture stirred at 25° C. for 2 hrs. LC-MS detected˜50% of the desired mass. The reaction mixture was quenched by addition of water (15.0 mL) at 0° C. and extracted with EtOAc (40.0 mL*3). The combined organic extracts were washed with brine (30.0 mL), dried over Na₂SO₄, filtered and concentrated under reduced pressure to give a residue. HPLC showed detection of 50.5% of the desired compound. The crude product was combined with a similar reaction run on a 1.80 g scale for further purification. The combine crude product was purified by Prep-HPLC (basic condition, column: Kromasil Eternity XT 250*80 mm*10 um; mobile phase: [water (0.05% ammonia hydroxide v/v)−ACN]; B %: 45%-70%, 17 min). Compound 9 (2.35 g, 5.63 mmol, 20.43% yield) was obtained as a brown oil. LC-MS (M+H)⁺: 418.3; HPLC purity: 50.5% (220 nm); ¹H NMR (400 MHz, DMSO-d₆): δ 7.39 (d, J=7.6 Hz, 1H), 6.87 (d, J=8.0 Hz, 1H), 3.61 (t, J=6.0 Hz, 2H), 3.58 (s, 3H), 2.80 (d, J=9.2 Hz, 1H), 2.67 (t, J=6.4 Hz, 2H), 2.59 (t, J=7.6 Hz, 3H), 2.49-2.44 (m, 1H), 2.30 (t, J=6.8 Hz, 2H), 2.12 (t, J=10.0 Hz. 1H), 1.96 (t, J=10.4 Hz, 1H). 1.85-1.73 (m, 5H), 1.66-1.57 (M. 1H). 1.48-1.44 (m, 1H), 1.43 (s, 9H), 1.40-1.35 (m, 1H): SFC chiral purity: 95.6%.

### Preparation of Compound 10

To a solution of compound 9 (2.35 g, 5.63 mmol, 1.00 eq) in MeOH (20.0 mL) was added a solution of LiOH·H₂O (354 mg, 8.44 mmol, 1.50 eq) in H₂O (5.00 mL) and mixture stirred at 25° C. for 6 hrs. LC-MS showed compound 9 was completely consumed and detected one main peak with the desired mass. The reaction mixture was concentrated under reduced pressure to give compound 10 (2.31 g, crude) as a brown solid. LC-MS (M+H)⁺: 404.1.

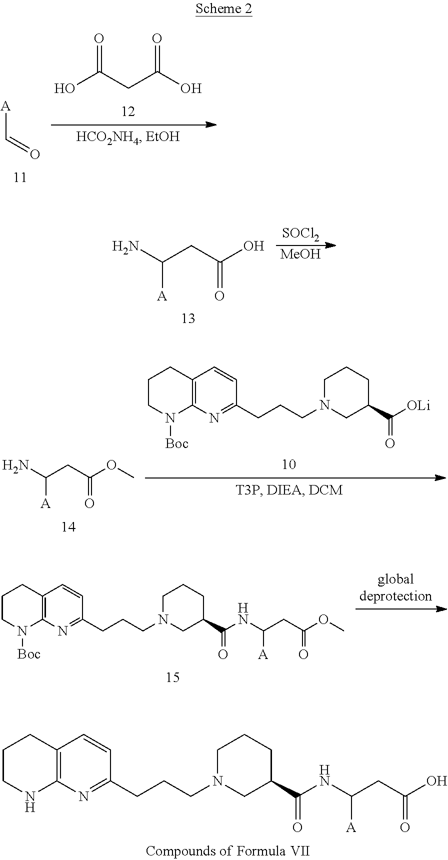

### FIG. 2

FIG. 2, Scheme 2, above illustrates a general procedures to prepare some compounds of Formula (VII).

### General Procedure for Preparation of Amino Acids 13

To a solution of aldehyde 11 (1.00 eq) and malonic acid 12 (1.10 eq) in EtOH (2.00 mL) was added ammonia and formic acid (2.00 eq). The mixture was stirred at 80° C. for 12 hrs. The reaction was monitored using TLC and LC-MS and the mixture was concentrated in vacuum to give crude product 13 whose structure was confirmed using LC-MS.

### General Procedure for Preparation of Amino Esters 14

To a solution of amino acid 13 (1.00 eq) in MeOH (2.00 mL) was added SOCl₂ (0.50 eq) and the mixture was stirred at 25° C. for 12 hrs. The reaction was monitored using TLC and LC-MS and the mixture was concentrated to obtain amino ester 14.

### General Procedure for Preparation of Amino Esters 15

To a solution of amino ester 14 (1.00 eq) and compound 10 (1.00 eq) in DCM (2.00 mL) was added T3P (1.00 eq) and DIEA (1.00 eq). The mixture was stirred at 25° C. for 5 hrs and monitored using TLC and LC-MS. The reaction mixture was diluted with H₂O (10.0 mL) and extracted with DCM (10.0 mL*3). The combined organic extracts were washed with brine (20.0 mL), dried over drying Na₂SO₄, filtered and concentrated give a residue. The residue was purified by Prep-HPLC (column: Phenomenex Gemini−NX C18 75*30 mm*3 um; mobile phase: water (10 mM NH₄HCO₃)−acetonitrile) to yield compound 15.

### General Procedure for Preparation of Some Compounds of Formula (VII)

Compound 15 (1.00 eq) was dissolved in a solution of HCl (121 eq) and the mixture was stirred at 60° C. for 2 hrs while being monitored by TLC and LC-MS. The reaction mixture was concentrated, and the residue was purified by Prep-HPLC (column: Phenomenex luna C18 80*40 mm*3 um; mobile phase: [water (0.05% HCl)−ACN]) to obtain a compound of Formula (VII).

SFC was used to separate isomers (enantiomers or diastereomers or epimers) when needed.

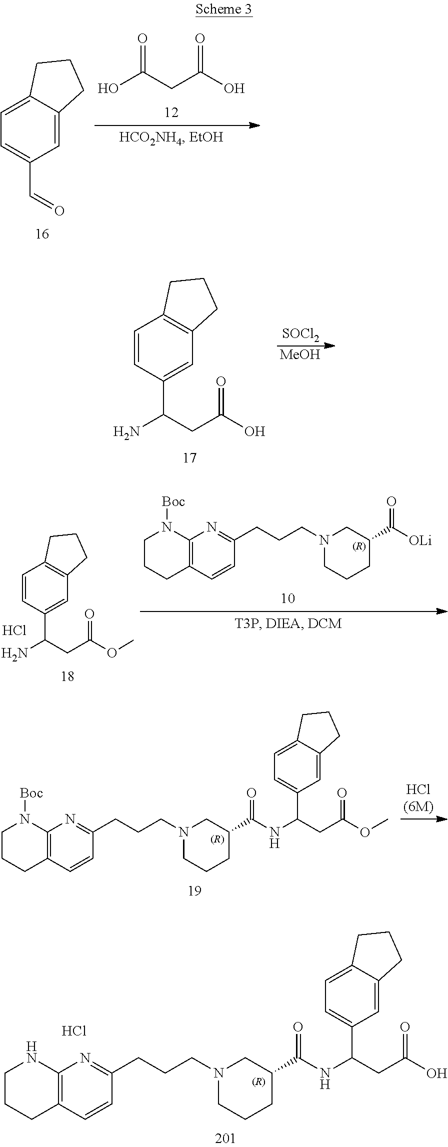

Scheme 3 illustrates the synthesis of compound 201, which exemplifies the synthesis of compounds of Formula (VII) shown in FIG. 2, Scheme 2.

### Preparation of 3-amino-3-(2,3-dihydro-1H-inden-5-yl)propanoic acid (17)

To a solution of 2,3-dihydro-1H-indene-5-carbaldehyde 16, (300 mg, 2.05 mmol, 1.00 eq) and malonic acid 12, 235 mg, 2.26 mmol, 235 uL, 1.10 eq) in EtOH (2.00 mL) was added ammonia formic acid (259 mg, 4.10 mmol, 2.00 eq). The mixture was stirred at 80° C. for 12 hrs when TLC (petroleum ether: ethyl acetate=5:1, R_{f}(P1)=0.80) indicated compound 16 was completely consumed. The mixture was concentrated give crude 3-amino-3-(2,3-dihydro-1H-inden-5-yl)propanoic acid 17 as a white solid, (200 mg) whose structure was confirmed by LC-MS.

### Preparation of methyl 3-amino-3-(2,3-dihydro-1H-inden-5-yl)propanoate hydrochloride (18)

To a solution of compound 17 (100 mg, 487 umol, 1.00 eq) in MeOH (2.00 mL) was added SOCl₂ (29.0 mg, 244 umol, 17.7 uL, 0.500 eq) and the mixture stirred at 25° C. for 12 hrs. LC-MS detected the presence of the desired mass. The reaction mixture was concentrated to yield crude methyl 3-amino-3-(2,3-dihydro-1H-inden-5-yl)propanoate hydrochloride 18 (70.0 mg, white solid). LC-MS (M+H)⁺: 220.2.

### Preparation of tert-butyl 7-(3-((3R)-3-((1-(2,3-dihydro-1H-inden-5-yl)-3-methoxy-3-oxopropyl)carbamoyl)piperidin-1-yl)propyl)-3,4-dihydro-1,8-naphthyridine-1(2H)-carboxylate (19)

To a solution of compound 18 (70.0 mg, 274 umol, 1.00 eq, HCl) and compound 10 (117 mg, 274 umol, 1.00 eq. LiOH) in DCM (2.00 mL) was added T3P (174 mg, 274 umol, 163 uL, 50.0% purity, 1.00 eq), DIEA (35.4 mg, 274 umol, 47.7 uL, 1.00 eq) and the mixture stirred at 25° C. for 5 hrs when LC-MS showed compound 18 was completely consumed. A main new peak of the correct mass was detected. The reaction mixture was diluted with H₂O (10.0 mL) and extracted with DCM (10.0 mL*3). The combined organic extracts were washed with brine (20.0 mL), dried over drying Na₂SO₄, filtered and concentrated under reduced pressure to give a residue which was purified by Prep-HPLC (column: Phenomenex Gemini−NX C18 75*30 mm*3 um; mobile phase: [water (10 mM NH₄HCO₃)−ACN]; B %: 15%-85%. 18 min) to yield compound 19 (40.0 mg, 66.1 umol, 24.2% yield) as a light-yellow oil. LC-MS (M+H)⁺: 605.6.

### Example 1: 3-(2,3-dihydro-1H-inden-5-yl)-3-((R)-1-(3-(5,6,7,8-tetrahydro-1,8-naphthyridin-2-yl)propyl)piperidine-3-carboxamido)propanoic acid hydrochloride (201)

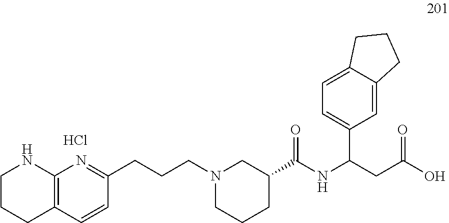 [SMILES: O=C(O)CC(NC(=O)[C@@H]1CCCN(CCCc2ccc3c(n2)NCCC3)C1)c1ccc2c(c1)CCC2 ; Cl]

Compound 19 (40.0 mg, 66.1 umol, 1.00 eq) was dissolved in HCl (6.00 M, 1.33 mL, 121 eq) and stirred at 60° C. for 2 hrs when LC-MS detected the desired product. The reaction mixture was concentrated and purified by Prep-HPLC (column: Phenomenex luna C18 80*40 mm*3 um; mobile phase: [water (0.05% HCl)−ACN]; B %: 0%-60%, 10 min) to provide compound 201 (24.03 mg, 44.8 umol, 67.7% yield, 98.3% purity, HCl) as a white solid. LC-MS (M+H)⁺: 491.4; ¹H NMR (400 MHz, DMSO-<4): δ 14.1-14.0 (m, 1H), 10.7-10.6 (m, 1H), 8.83-8.65 (m, 1H), 8.25-7.84 (m, 1H), 7.62 (t, J=6.8 Hz, 1H), 7.16-7.10 (m, 2H), 7.09-7.03 (m, 1H), 6.70-6.64 (m, 1H), 5.21-5.08 (m, 1H), 3.36-3.21 (m, 5H), 3.08-3.00 (m, 2H), 2.96-2.69 (m, 12H), 2.68-2.61 (m, 2H), 2.20-2.05 (m, 2H), 2.02-1.96 (m, 2H), 1.93-1.81 (m, 4H). Prep-SFC (column: DAICEL CHIRALPAK AS (250 mm*30 mm, 10 um); mobile phase: [0.1% NH₃H₂O IPA]; B %: 65%-65%, 4.0 min; 20 min, SFC (EW24694-70-P1A_A13), RT (peak 1)=0.541 min, RT (peak 2)=1.521 min).

The stereoisomers of compound 201 were resolved using prep SFC (Prep-SFC (column: DAICEL CHIRALPAK AS (250 mm*30 mm, 10 um); mobile phase: [0.1% NH₃H₂O IPA]; B %: 65%-65%, 4.0 min; 20 min, SFC, RT (peak 1)=0.541 min, RT (peak 2)=1.521 min) to provide two isomers: compound 201-A and compound 201-B.

Compound 201-A: ¹H NMR (400 MHz, CDCl₃): δ 10.6 (br s, 1H), 9.28 (br s, 1H), 7.33 (s, 1H), 7.23-7.17 (m, 2H), 7.11 (d, J=8.0 Hz, 1H). 6.24 (d, J=7.6 Hz, 1H), 5.45-5.43 (m, 1H), 3.43-3.36 (m, 2H), 3.30-3.26 (m, 1H), 3.07-2.92 (m, 3H), 2.91-2.75 (m, 7H), 2.70-2.64 (m, 3H), 2.55-2.39 (m, 3H), 2.14-2.08 (m, 1H), 2.05-1.99 (m, 2H), 1.89-1.82 (m, 2H), 1.79-1.73 (m, 1H), 1.66-1.53 (m, 2H), 1.33-1.25 (m, 2H); LC-MS (M+H)⁺: 491.2; HPLC purity: 85.3% (220 nm); SFC chiral purity: 100%.

Compound 201-B: ¹H NMR (400 MHz, CDCl₃): δ 8.84 (br s, 1H), 8.40 (br s, 1H), 7.30 (d, J=6.8 Hz, 1H), 7.21 (s, 1H), 7.15-7.10 (m, 2H), 6.48 (d, J=7.2 Hz, 1H), 5.34-5.33 (m, 1H), 4.06-4.00 (m, 1H), 3.73 (br s, 1H), 3.51-3.40 (m, 3H), 3.24-3.15 (m, 2H), 3.05-2.98 (m, 1H), 2.95-2.78 (m, 8H), 2.75-2.71 (m, 3H), 2.37-2.29 (m, 1H), 2.06-2.00 (M, 2H), 1.95-1.87 (m, 3H), 1.75-1.64 (m, 1H), 1.49-1.39 (m, 1H), 1.34-1.24 (m, 3H), 1.21 (d, J=6.0 Hz, 2H); LC-MS (M+H)⁺: 491.2; HPLC purity: 91.5% (215 nm); SFC chiral purity: 97.8%.

### Example 2: (S)-3-phenyl-3-((R)-1-(3-(5,6,7,8-tetrahydro-1,8-naphthyridin-2-yl)propyl)piperidine-3-carboxamido)propanoic acid hydrochloride (202)

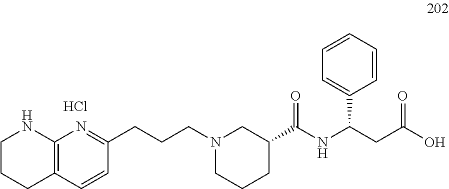 [SMILES: O=C(O)C[C@H](NC(=O)[C@@H]1CCCN(CCCc2ccc3c(n2)NCCC3)C1)c1ccccc1 ; Cl]

Following the procedure illustrated in FIG. 2, Scheme 2, yielded compound 202. LC-MS (M+H)⁺: 451.4: ¹H NMR (400 MHz, DMSO-d₆) δ 14.1 (s, 1H), 10.9 (br s, 1H), 8.78 (d, J=8.4 Hz, 1H), 8.01 (s, 1H), 7.63 (d, J=7.2 Hz, 1H), 7.33-7.30 (m, 4H), 7.24-7.22 (m, 1H), 6.68 (d, J=7.2 Hz, 1H), 5.17-5.12 (m, 1H), 3.43-3.41 (m, 4H), 3.06-3.02 (m, 2H), 2.93-2.89 (m, 2H), 2.75-2.68 (m, 5H), 2.68-2.65 (m, 2H), 2.12-2.09 (m, 2H), 1.97-1.90 (m, 2H), 1.83-1.80 (m, 3H), 1.33-1.27 (M, 1H); HPLC purity: 99.5% (215 nm); SFC chiral purity: 100%.

### Example 3: 3-((R)-1-(3-(5,6,7,8-tetrahydro-1,8-naphthyridin-2-yl)propyl)piperidine-3-carboxamido)-3-(3-(trifluoromethyl)phenyl)propanoic acid hydrochloride (203)

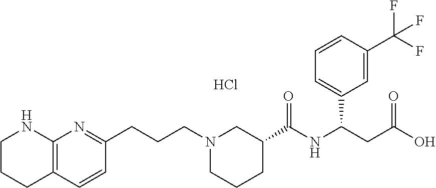 [SMILES: O=C(O)C[C@H](NC(=O)[C@@H]1CCCN(CCCc2ccc3c(n2)NCCC3)C1)c1cccc(C(F)(F)F)c1 ; Cl]

Following the procedure illustrated in FIG. 2, Scheme 2, yielded compound 203 which was resolved using the general procedure of Example 1 to yield two isomers compound 203-A and compound 203-B.

Compound 203-A: ¹H NMR (400 MHz, DMSO-d₆): δ 8.76 (d, J=8.0 Hz, 1H), 7.74 (s, 1H), 7.64 (d, J=7.2 Hz, 1H), 7.58-7.52 (m, 2H), 7.13 (d, J=7.6 Hz, 1H), 6.31 (d, J=7.2 Hz, 1H), 5.24 (q, J=6.0 Hz, 1H), 3.25-3.19 (m, 3H), 2.69 (d, J=6.4 Hz, 2H), 2.64-2.56 (m, 3H), 2.44-2.36 (m, 3H), 2.34-2.25 (m, 3H), 2.24-2.15 (m, 1H), 1.79-1.65 (m, 4H), 1.62-1.50 (m, 3H), 1.46-1.38 (m, 1H): LC-MS (M+H)⁺: 519.1.

Compound 203-B: ¹H NMR (400 MHz, DMSO-d₆) δ 8.86-8.78 (m, 1H), 7.66-7.46 (m, 4H), 7.07 (d, J=7.2 Hz, 1H), 6.92-6.73 (m, 1H), 6.27 (d, J=7.2 Hz, 1H), 5.20 (q, J=7.2 Hz, 1H), 3.25-3.21 (m, 3H), 2.76-2.68 (m, 1H), 2.65-2.57 (m, 4H), 2.47-2.42 (m, 2H), 2.41-2.32 (m, 2H), 2.31-2.24 (m, 2H), 2.04-1.95 (m, 1H), 1.78-1.67 (m, 4H), 1.66-1.57 (m, 2H), 1.47-1.30 (m, 2H); LC-MS (M+H)⁺: 519.1.

### Example 4: 3-((R)-1-(3-(5,6,7,8-tetrahydro-1,8-naphthyridin-2-yl)propyl)piperidine-3-carboxamido)-3-(5,6,7,8-tetrahydronaphthalen-2-yl)propanoic acid (204)

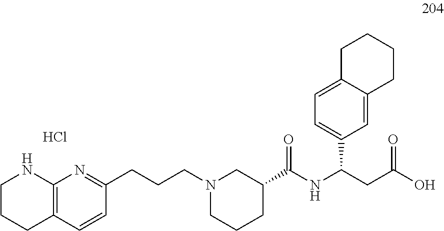 [SMILES: O=C(O)C[C@H](NC(=O)[C@@H]1CCCN(CCCc2ccc3c(n2)NCCC3)C1)c1ccc2c(c1)CCCC2 ; Cl]

Following the procedure illustrated in FIG. 2, Scheme 2, yielded compound 204 which was resolved using the general procedure of Example 1 to yield two isomers compound 204-A and compound 204-B.

Compound 204-A: ¹H NMR (400 MHz, CDCl₃) δ 10.7-10.6 (m, 1H), 9.96-9.75 (m, 1H), 7.18 (d, J=6.8 Hz, 1H), 7.12 (d, J=7.6 Hz, 1H), 7.08 (s, 1H), 6.92 (d, J=8.0 Hz, 1H), 6.21 (d, J=7.2 Hz, 1H), 5.46-5.35 (m, 1H), 4.78-4.51 (m, 1H), 3.47-3.32 (m, 2H), 2.95-2.80 (m, 5H), 2.71-2.55 (m, 8H), 2.52-2.37 (m, 3H), 2.33-2.30 (m, 1H), 2.01-1.90 (m, 2H), 1.89-1.84 (m, 2H), 1.64-1.55 (M, 2H), 1.32-1.21 (s. 6H); LC-MS (M+H)⁺: 505.6.

Compound 204-B: ¹H NMR (400 MHz, CDCl₃) δ 10.7 (br s, 1H), 9.34 (br s, 1H), 7.19-7.15 (m, 3H), 6.95 (d, J=8.0 Hz, 1H), 6.24 (d, J=7.2 Hz, 1H), 5.43-5.41 (m, 1H), 3.39 (br s. 2H), 3.32-3.29 (m, 1H), 3.12-2.95 (m, 3H), 2.93-2.77 (m, 3H), 2.74-2.60 (m, 7H), 2.51-2.35 (m, 3H), 2.22-1.99 (m, 4H), 1.90-1.81 (m, 2H), 1.60-1.46 (m, 2H), 1.39-1.24 (m, 4H); LC-MS: (M+H)⁺: 505.5.

### Example 5: (S)-3-(3-bromophenyl)-3-((R)-1-(3-(5,6,7,8-tetrahydro-1,8-naphthyridin-2-yl)propyl)piperidine-3-carboxamido)propanoic acid hydrochloride (205)

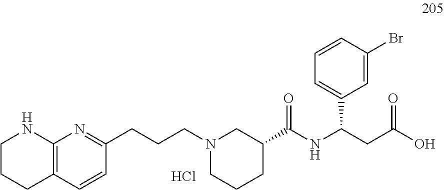 [SMILES: O=C(O)C[C@H](NC(=O)[C@@H]1CCCN(CCCc2ccc3c(n2)NCCC3)C1)c1cccc(Br)c1 ; Cl]

Following the procedure illustrated in FIG. 2, Scheme 2, yielded compound 205. LC-MS (M+H)⁺: 531.0; ¹H NMR (400 MHz, DMSO-d₆) δ 8.79 (br s, 1H), 8.12-7.66 (m, 1H), 7.64-7.53 (m, 1H), 7.49 (s, 1H), 7.44-7.42 (m, 1H), 7.34-7.25 (m, 2H), 6.63 (d, J=6.0 Hz, 1H), 5.15-5.09 (m, 1H), 3.51-3.40 (m, 5H), 3.05 (t, 8.0 Hz, 3H), 2.92-2.91 (m, 2H), 2.78-2.67 (m, 6H), 2.17-2.06 (m, 2H), 1.95-1.77 (m, 5H).

### Example 6: (R)-3-(naphthalen-2-yl)-3-((R)-1-(3-(5,6,7,8-tetrahydro-1,8-naphthyridin-2-yl)propyl)piperidine-3-carboxamido)propanoic acid hydrochloride (206)

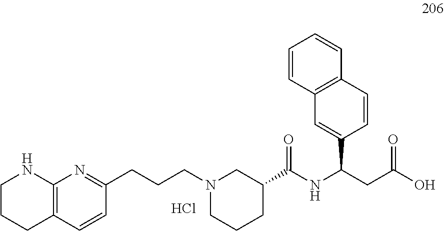 [SMILES: O=C(O)C[C@@H](NC(=O)[C@@H]1CCCN(CCCc2ccc3c(n2)NCCC3)C1)c1ccc2ccccc2c1 ; Cl]

Following the procedure illustrated in FIG. 2, Scheme 2, yielded compound 206. LC-MS (M+H)⁺: 501.3: ¹H NMR (400 MHz, DMSO-d₆) δ 14.1 (s, 1H), 10.8 (s, 1H), 8.88 (d, J=8.4 Hz, 1H), 7.98 (s, 1H), 7.89-7.86 (m, 3H), 7.77 (s, 1H), 7.62 (d, J=7.2 Hz, 1H), 7.54-7.44 (m, 3H), 6.67 (d, J=7.6 Hz, 1H), 5.31 (q, J=7.2 Hz, 1H), 3.41-3.38 (m, 2H), 3.13-2.87 (m, 5H), 2.88-2.81 (m, 7H), 2.21-2.06 (m, 2H), 2.02-1.75 (m, 5H), 1.41-1.20 (m, 2H); HPLC purity: 93.9% (220 nm); SFC chiral purity; 100%.

### Example 7: (S)-3-(naphthalen-2-yl)-3-((R)-1-(3-(5,6,7,8-tetrahydro-1,8-naphthyridin-2-yl)propyl)piperidine-3-carboxamido)propanoic acid hydrochloride (207)

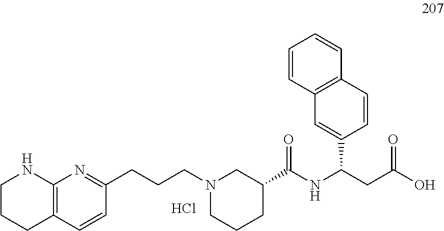 [SMILES: O=C(O)C[C@H](NC(=O)[C@@H]1CCCN(CCCc2ccc3c(n2)NCCC3)C1)c1ccc2ccccc2c1 ; Cl]

Following the procedure illustrated in FIG. 2, Scheme 2, yielded compound 207. LC-MS (M+H)⁺: 501.1: ¹H NMR (400 MHz, DMSO-d₆) δ 14.3 (s, 1H), 11.0 (s, 1H), 8.90 (d, J=8.0 Hz, 1H), 8.05 (s, 1H), 7.89-7.87 (m, 3H), 7.80 (s, 1H), 7.59 (d, J=7.2 Hz, 1H), 7.52-7.46 (m, 3H), 6.64 (d, J=7.2 Hz, 1H), 5.31 (q, J=7.2 Hz, 1H), 3.41-3.38 (m, 1H), 3.09-2.96 (m, 3H), 2.95-2.91 (m, 2H), 2.86-2.69 (m, 8H), 2.20-2.12 (m, 2H), 2.03-2.88 (m, 2H), 2.86-2.70 (m, 4H), 1.51-1.45 (m, 1H); HPLC purity: 93.7% (220 nm); SFC chiral purity: 100%.

### Example 8: 3-(3-fluoro-5-(trifluoromethyl)phenyl)-3-((R)-1-(3-(5,6,7,8-tetrahydro-1,8-naphthyridin-2-yl)propyl)piperidine-3-carboxamido)propanoic acid (208)

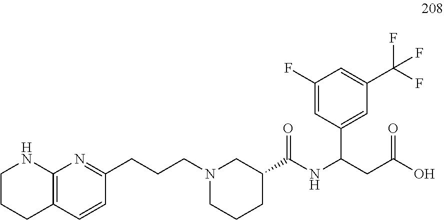 [SMILES: O=C(O)CC(NC(=O)[C@@H]1CCCN(CCCc2ccc3c(n2)NCCC3)C1)c1cc(F)cc(C(F)(F)F)c1]

Following the procedure illustrated in FIG. 2, Scheme 2, yielded compound 208 which was separated following the general procedure of Example 1 to yield two isomers compound 208-A and compound 208-B.

Compound 208-A: ¹H NMR (400 MHz, CDCl₃) δ 10.6 (s, 1H), 10.04 (br s, 1H), 7.49 (s, 1H), 7.29 (d, J=10 Hz, 1H), 7.20 (d, J=7.2 Hz, 1H), 7.10 (d, J=8.4 Hz, 1H), 6.23 (d, J=7.2 Hz, 1H), 5.55-5.52 (m, 1H), 3.39 (t, J=5.20 Hz, 2H), 3.02 (s, 2H), 2.91-2.86 (m, 3H), 2.67 (t, J=6.0 Hz, 4H), 2.56-2.51 (m, 1H), 2.48-2.43 (m, 3H), 2.01 (d, J=12.4 Hz, 2H), 1.90-1.79 (m, 6H); LC-MS: (M+H)⁺: 537.3. HPLC purity: 100% (220 nm): SFC chiral purity: 100%.

Compound 208-B: ¹H NMR (400 MHz, CDCl₃) δ 9.20 (br s, 1H), 7.60 (s, 1H), 7.46-7.30 (m, 1H), 7.24 (s, 1H), 7.11 (d, J=8 Hz, 1H), 6.33 (d, J=7.2 Hz, 1H), 5.41 (s, 1H), 3.49-3.37 (m, 2H), 3.29-3.00 (m, 2H), 2.92-2.84 (m, 5H), 2.77-2.49 (m, 5H), 2.42-2.30 (br s, 1H), 2.20-1.65 (m, 3H), 1.86 (t, J=5.2 Hz, 3H), 1.75 (s, 2H); LC-MS: (M+H)⁺: 537.7: SFC chiral purity: 100%.

### Example 9: 3-(3-phenoxyphenyl)-3-((R)-1-(3-(5,6,7,8-tetrahydro-1,8-naphthyridin-2-yl)propyl)piperidine-3-carboxamido)propanoic acid (209)

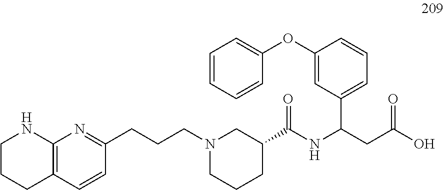 [SMILES: O=C(O)CC(NC(=O)[C@@H]1CCCN(CCCc2ccc3c(n2)NCCC3)C1)c1cccc(Oc2ccccc2)c1]

Following the procedure illustrated in FIG. 2, Scheme 2, yielded compound 209 which was resolved following the general procedure of Example 1 to yield two isomers compound 209-A and compound 209-B.

Compound 209-A: ¹H NMR (400 MHz, DMSO-d₆) δ 8.65-8.63 (m, 1H), 7.36 (t, J=7.6 Hz, 2H), 7.30 (t, J=7.6 Hz, 1H), 7.13 (t, J=7.6 Hz, 1H), 7.06 (d, J=7.6 Hz, 2H), 6.99-6.95 (m, 3H), 6.84 (d, J=7.6 Hz, 1H), 5.16-5.11 (m, 1H), 3.26-3.22 (m, 4H), 2.64-2.56 (m, 4H), 2.46-2.41 (m, 3H), 2.33-2.29 (m, 2H), 2.23 (t, J=7.2 Hz, 2H), 2.08-1.95 (m, 2H), 1.79-1.66 (m, 4H), 1.59-1.58 (m, 2H); LC-MS (M+H)⁺: 543.4.

Compound 209-B: ¹H NMR (400 MHz, DMSO-d₆) δ 8.75 (brs, 1H), 7.39 (t, J=8.0 Hz, 2H), 7.32 (t, J=8.0 Hz, 1H), 7.14 (d, J=7.6 Hz, 1H), 7.12-7.08 (m, 2H), 7.01-6.98 (m, 3H), 6.84 (d, J=8.0 Hz, 1H), 6.40 (brs, 1H), 5.15-5.13 (m, 1H), 3.33-3.25 (m, 4H), 3.06-2.96 (m, 3H), 2.91-2.79 (m, 2H), 2.68-2.63 (m, 4H), 2.57-2.54 (m, 2H), 2.01 (brs, 2H), 1.87-1.75 (m. 5H), 1.52-1.49 (m, 1H); LC-MS (M+H)⁺: 543.4.

### Example 10: 3-(2-isopropyl-5-methoxyphenyl)-3-((R)-1-(3-(5,6,7,8-tetrahydro-1,8-naphthyridin-2-yl)propyl)piperidine-3-carboxamido)propanoic acid hydrochloride (210)

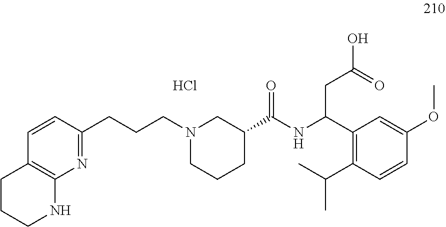 [SMILES: COc1ccc(C(C)C)c(C(CC(=O)O)NC(=O)[C@@H]2CCCN(CCCc3ccc4c(n3)NCCC4)C2)c1 ; Cl]

Following the procedure illustrated in FIG. 2, Scheme 2, yielded compound 210. ¹H NMR (400 MHz, DMSO-d₆) δ 14.1 (brs, 1H), 10.9-10.7 (m, 1H), 8.79-8.75 (m, 1H), 8.09-7.99 (m, 1H), 7.62 (t, J=8.0 Hz, 1H), 7.16 (d, J=8.0 Hz, 1H), 6.97-6.89 (m, 1H), 6.83-6.76 (m, 1H), 6.69-6.60 (m, 1H), 5.52-5.44 (m, 1H), 3.71-3.70 (m, 3H), 3.42-3.34 (m, 6H), 3.24-3.15 (m. 1H), 3.09-2.98 (m, 2H), 2.93-2.2.86 (m, 2H), 2.77-2.71 (m, 4H), 2.67-2.59 (m, 1H), 2.20-2.07 (m, 2H), 1.97-1.90 (m, 1H), 1.86-1.81 (m, 2H), 1.55-1.42 (m, 1H), 1.23 (s, 2H), 1.18 (d, J=8.0 Hz, 6H): LC-MS (M+H)⁺: 523.2.

### Example 11: 3-(2,3-dihydro-1H-inden-4-yl)-3-((R)-1-(3-(5,6,7,8-tetrahydro-1,8-naphthyridin-2-yl)propyl)piperidine-3-carboxamido)propanoic acid (211)

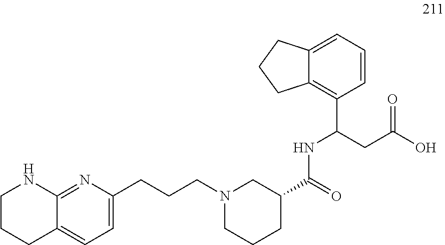 [SMILES: O=C(O)CC(NC(=O)[C@@H]1CCCN(CCCc2ccc3c(n2)NCCC3)C1)c1cccc2c1CCC2]

Following the procedure illustrated in FIG. 2, Scheme 2, yielded compound 211 which was resolved following the general procedure of Example 1 to yield two isomers compound 211-A and compound 211-B.

Compound 211-A: ¹H NMR (400 MHz, DMSO-4) δ 8.53-8.42 (m, 1H), 7.10-7.01 (m. 4H), 6.57-6.51 (m, 1H), 6.47 (d, J=7.2 Hz, 1H), 5.21-5.16 (m, 1H), 3.24-3.22 (m, 2H), 2.93-2.81 (m, 5H), 2.69-2.66 (m, 1H), 2.62-2.56 (m, 5H), 2.33-2.32 (m, 2H), 2.29-2.24 (m, 2H), 2.03-1.92 (m, 4H), 1.77-1.66 (m, 4H), 1.64-1.54 (m, 2H), 1.26-1.24 (m, 3H): LC-MS (M+H)⁺: 419.5.

Compound 211-B: ¹H NMR (400 MHz, DMSO-d₆) δ 8.72 (d, J=8.0 Hz, 1H), 7.18-7.05 (m, 4H), 6.34 (d, J=6.4 Hz, 1H), 5.26-5.19 (m, 1H), 3.251-3.20 (m, 2H), 3.04-2.96 (m, 3H), 2.94-2.79 (m, 8H), 2.64-2.60 (m, 4H), 2.03-1.96 (m, 4H), 1.84-1.71 (m, 4H), 1.59-1.44 (m, 2H), 1.26-1.24 (m, 3H); LC-MS (M+H)⁺: 419.5.

### Example 12: 3-((R)-1-(3-(5,6,7,8-tetrahydro-1,8-naphthyridin-2-yl)propyl)piperidine-3-carboxamido)-3-(5,6,7,8-tetrahydronaphthalen-1-yl)propanoic acid (212)

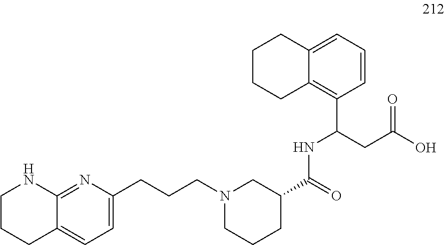 [SMILES: O=C(O)CC(NC(=O)[C@@H]1CCCN(CCCc2ccc3c(n2)NCCC3)C1)c1cccc2c1CCCC2]

Following the procedure illustrated in FIG. 2, Scheme 2, yielded which was resolved following the general procedure of Example 1 to yield two isomers compound 212-A and compound 212-B.

Compound 212-A: ¹H NMR: (400 MHz, DMSO-d₆) δ 8.52 (s, 1H), 7.13 (d, J=2.4 Hz, 1H), 7.02 (d, J=6.4 Hz, 2H), 6.90 (d, J=6.4 Hz, 1H), 6.51 (s, 1H), 6.24 (d, J=6.8 Hz, 1H), 5.33 (d, J=5.6 Hz, 1H), 3.30-3.19 (m, 3H), 2.90-2.80 (m, 1H), 2.75-2.55 (m, 7H), 2.44-2.38 (m, 1H), 2.35-2.20 (m, 3H), 2.08-1.90 (m, 2H), 1.80-1.60 (m, 8H), 1.59-1.50 ((m, 1H), 1.48-1.25 (m, 2H), 1.15-1.10 (m, 3H): LC-MS (M+H)⁺: 505.5.

Compound 212-B: ¹H NMR: (400 MHz, DMSO-d₆) δ 7.52 (d, J=7.6 Hz, 1H), 7.12-7.01 (m, 2H), 6.92 (d, J=7.2 Hz, 1H), 6.58 (d, J=7.2 Hz, 11H), 5.33 (t, J=6.4 Hz, 1H), 3.37-3.33 (m, 2H), 3.31-3.26 (m, 1H), 3.07-3.01 (m, 2H), 2.93-2.84 (m, 1H), 2.82-2.73 (m, 2H), 2.73-2.61 (m, 7H), 2.60-2.56 (m, 2H), 2.06-1.93 (m, 2H), 1.85-1.57 (m, 9H), 1.53-1.35 (m, 1H), 1.22-1.12 (m, 2H): LC-MS (M+H)⁺: 505.5.

### Example 13: 3-(2,3-dihydrobenzofuran-5-yl)-3-((R)-1-(3-(5,6,7,8-tetrahydro-1,8-naphthyridin-2-yl)propyl) piperidine-3-carboxamido)propanoic acid hydrochloride (213)

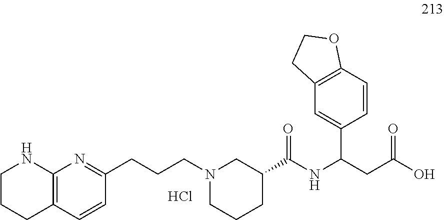 [SMILES: O=C(O)CC(NC(=O)[C@@H]1CCCN(CCCc2ccc3c(n2)NCCC3)C1)c1ccc2c(c1)CCO2 ; Cl]

Following the procedure illustrated in FIG. 2, Scheme 2, yielded compound 213. ¹H NMR (400 MHz, DMSO-d₆) δ 14.3 (br s, 1H), 10.9 (br s, 1H), 8.68 (s, 1H), 8.06 (s, 1H), 7.65-7.57 (m, 1H), 7.16 (d, J=9.2 Hz, 1H), 7.01 (t, J=6.4 Hz, 1H), 6.71-6.61 (m, 2H), 5.15-5.00 (m, 1H), 4.48 (t, J=8.8 Hz, 2H), 3.62-3.56 (m, 1H), 3.18-2.98 (m, 5H), 2.93-2.84 (m, 2H), 2.81-2.69 (m, 5H), 2.68-2.56 (m, 2H), 2.48-2.40 (m, 2H), 2.20-2.07 (m, 2H), 1.96-1.74 (m, 5H), 1.52-1.25 (m, 1H): LC-MS (M+H)⁺: 493.3.

### Example 14: (S)-3-(benzo[d][1,3]dioxol-5-yl)-3-((R)-1-(3-(5,6,7,8-tetrahydro-1,8-naphthyridin-2-yl)propyl) piperidine-3-carboxamido)propanoic acid (214)

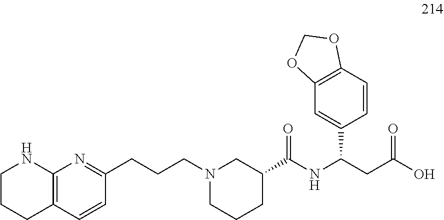 [SMILES: O=C(O)C[C@H](NC(=O)[C@@H]1CCCN(CCCc2ccc3c(n2)NCCC3)C1)c1ccc2c(c1)OCO2]

Following the procedure illustrated in FIG. 2, Scheme 2, yielded compound 214. ¹H NMR (400 MHz, CDCl₃) δ 10.7 (br s, 1H), 9.62 (br s, 1H), 7.20 (d, J=7.2 Hz, 1H), 6.92 (d, J=1.6 Hz, 1H), 6.86 (dd, J₁=8.0 Hz, J₂=1.2 Hz, 1H), 6.70 (d, J=8.4 Hz, 1H), 6.24 (d, J=7.2 Hz, 1H), 5.88-5.87 (m, 2H), 5.36 (br s, 1H), 3.41 (t, J=5.6 Hz, 2H), 2.93-2.89 (m, 4H), 2.79 (d, J=4.4 Hz, 2H), 2.69 (t, J=6.0 Hz, 4H), 2.56-2.55 (m, 2H), 2.40-2.38 (m, 1H), 1.90-1.86 (m, 5H), 1.82-1.79 (m, 1H), 1.67 (br s, 2H): LC-MS (M+H)⁺: 495.4.

### Example 15: 3-(quinoxalin-6-yl)-3-((R)-1-(3-(5,6,7,8-tetrahydro-1,8-naphthyridin-2-yl)propyl)piperidine-3-carboxamido)propanoic acid (215)

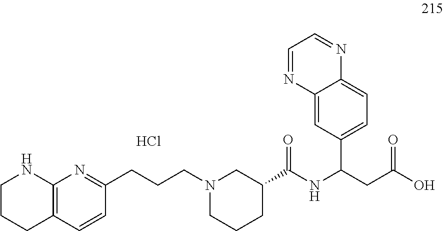 [SMILES: O=C(O)CC(NC(=O)[C@@H]1CCCN(CCCc2ccc3c(n2)NCCC3)C1)c1ccc2nccnc2c1 ; Cl]

Following the procedure illustrated in FIG. 2, Scheme 2, yielded compound 215 which was resolved following the general procedure of Example 1 to yield two isomers compound 215-A and compound 215-B.

Compound 215-A: ¹H NMR (400 MHz, DMSO-d₆) δ 14.18 (s. 1H), 10.96 (s. 1H), 9.03 (d, J=8.0 Hz, 1H), 8.93 (d, J=3.6 Hz, 2H), 8.06-8.03 (m, 1H), 7.96 (s, 1H), 7.86-7.84 (m, 1H), 7.62 (d, J=7.2 Hz, 1H), 6.68 (d, J=7.2 Hz, 1H), 5.39-5.37 (m, 1H), 3.49-3.43 (m, 4H), 3.04-2.85 (m, 4H), 2.83-2.75 (m, 4H), 2.73-2.72 (m, 4H), 2.18-2.11 (m, 2H), 1.99-1.93 (m, 2H), 1.83-1.80 (m, 3H); LC-MS (M+H)⁺: 503.4.

Compound 215-B: ¹H NMR (400 MHz, DMSO-d₆) δ 14.12 (s, 1H), 10.80 (s, 1H), 9.01-8.99 (m, 1H), 8.94-8.93 (m, 2H), 8.09-8.07 (m, 1H), 8.00 (s, 1H), 7.88-7.86 (m, 1H), 7.60 (d, J=7.2 Hz, 1H), 6.65 (d, J=7.2 Hz, 1H), 5.38 (q, J=8.0 Hz, 1H), 3.47-3.42 (m, 4H), 3.03 (m, 2H), 2.85-2.74 (m, 6H), 2.73-2.72 (m, 4H), 2.12-2.09 (m, 2H), 1.99-1.92 (m, 2H), 1.81-1.79 (m, 3H); LC-MS (M+H)⁺: 503.4.

### Example 16: 3-(chroman-5-yl)-3-((R)-1-(3-(5,6,7,8-tetrahydro-1,8-naphthyridin-2-yl)propyl)piperidine-3-carboxamido)propanoic acid (216)

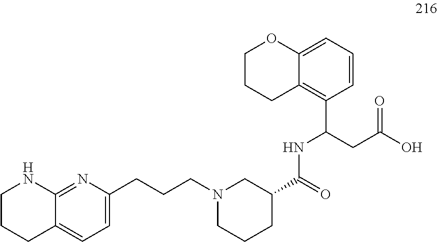 [SMILES: O=C(O)CC(NC(=O)[C@@H]1CCCN(CCCc2ccc3c(n2)NCCC3)C1)c1cccc2c1CCCO2]

Following the procedure illustrated in FIG. 2, Scheme 2, yielded compound 216 which was resolved following the procedure of Example 1 to yield compound 216-A and compound 216-B.

Compound 216-A: ¹H NMR (400 MHz, DMSO-d₆) δ 7.04 (t, J=7.6 Hz, 1H), 6.84 (d, J=7.6 Hz, 1H), 6.62 (dd, J=8.4 Hz. J₂=1.2 Hz, 1H), 6.52 (s, 2H), 5.28 (q, J=7.2 Hz, 1H), 4.12-3.97 (m, 2H), 3.07-3.00 (m, 3H), 2.95-2.86 (m, 3H), 2.84-2.78 (m, 2H), 2.73-2.65 (m. 4H), 2.60 (d, J=7.2 Hz, 2H), 2.34-2.31 (m, 1H), 2.06-1.84 (m, 6H), 1.83-1.74 (m, 4H), 1.24-1.22 (m, 2H); LC-MS (M+H)⁺: 507.5.

Compound 216-B: ¹H NMR (400 MHz, DMSO-d₆) δ 8.78-8.70 (m, 1H), 7.05 (t, J=7.6 Hz, 1H), 6.88 (d, J=7.6 Hz, 1H), 6.62 (dd, J=8.0 Hz, 1H), 6.52 (s, 2H), 5.39-5.20 (m, 1H), 4.12-3.97 (m, 2H), 3.06-2.97 (m, 3H), 2.94-2.82 (m, 4H), 2.80-2.71 (m, 2H), 2.69-2.57 (m, 5H), 2.34-2.30 (m, 1H), 2.04-1.85 (m, 6H), 1.84-1.74 (m, 4H), 1.28-1.20 (m, 2H); LC-MS (M+H)⁺: 507.5.

### Example 17: 3-(2,3-dihydrobenzofuran-6-yl)-3-((R)-1-(3-(5,6,7,8-tetrahydro-1,8-naphthyridin-2-yl)prop yl)piperidine-3-carboxamido)propanoic acid hydrochloride (217)

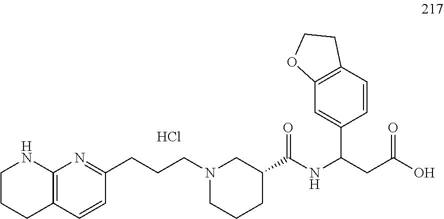 [SMILES: O=C(O)CC(NC(=O)[C@@H]1CCCN(CCCc2ccc3c(n2)NCCC3)C1)c1ccc2c(c1)OCC2 ; Cl]

Following the procedure illustrated in FIG. 2. Scheme 2, yielded compound 217. ¹H NMR (400 MHz, DMSO-d₆) δ 14.3-14.1 (m, 1H), 8.71 (s, 1H), 8.02 (s, 1H), 7.65-7.53 (m, 1H), 7.17-7.06 (m, 1H), 6.82-6.58 (m, 3H), 5.15-5.01 (m, 1H), 4.49 (t, J=8.4 Hz, 2H), 3.47-3.40 (m, 4H), 3.18-2.97 (m, 5H), 2.95-2.85 (m, 2H), 2.80-2.67 (m, 4H), 2.66-2.56 (m, 2H), 2.22-2.08 (m, 2H), 1.93-1.76 (m, 4H), 1.52-1.20 (m, 2H): LC-MS (M+H)⁺: 493.2.

### Example 18: 3-(benzo[d][1,3]dioxol-4-yl-3-((R)-1-(3-(5,6,7,8-tetrahydro-1,8-naphthyridin-2-yl)propyl)piperidine-3-carboxamido)propanoic acid hydrochloride (218)

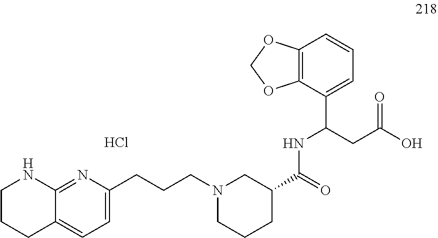 [SMILES: O=C(O)CC(NC(=O)[C@@H]1CCCN(CCCc2ccc3c(n2)NCCC3)C1)c1cccc2c1OCO2 ; Cl]

Following the procedure illustrated in FIG. 2, Scheme 2, yielded compound 218. ¹H NMR (400 MHz, DMSO-d₆) δ 11.23 (br s, 1H), 10.95 (s, 1H), 8.74 (d, J=7.6 Hz, 1H), 8.06 (s, 1H), 7.62 (d, J=7.2 Hz, 1H), 6.80-6.75 (m, 3H), 6.68 (d, J=7.2 Hz, 1H), 6.00 (d, J=9.2 Hz, 1H), 5.28-5.22 (m, 1H), 3.42-3.37 (m, 4H), 3.05 (br s, 2H), 2.98-2.88 (m, 2H), 2.81-2.68 (m, 5H), 2.66-2.62 (m, 2H), 2.18-2.08 (m, 2H), 2.00-1.91 (m, 2H), 1.82-1.74 (m, 3H), 1.39-1.29 (m. 1H); LC-MS (M+H)⁺: 495.2.

### Example 19: 3-(2,3-dihydrobenzofuran-4-yl)-3-((R)-1-(3-(5,6,7,8-tetrahydro-1,8-naphthyridin-2-yl)propyl) piperidine-3-carboxamido)propanoic acid hydrochloride (219)

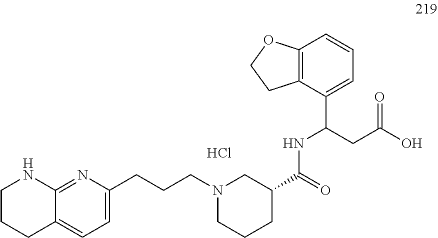 [SMILES: O=C(O)CC(NC(=O)[C@@H]1CCCN(CCCc2ccc3c(n2)NCCC3)C1)c1cccc2c1CCO2 ; Cl]

Following the procedure illustrated in FIG. 2, Scheme 2, yielded compound 219. ¹H NMR (400 MHz, DMSO-d₆) δ 10.66-10.50 (m, 11H), 8.77-8.73 (m, 1H), 7.99 (br s, 1H), 7.60 (t, J=6.4 Hz, 1H), 7.08-7.03 (m, 1H), 6.78 (t, J=8.4 Hz, 1H), 6.67-6.60 (m, 2H), 5.09 (q, J=7.6 Hz, 1H), 4.50 (t, J=8.8 Hz, 1H), 3.41-3.40 (m, 4H), 3.23-3.16 (m, 3H), 3.05-3.01 (m, 2H), 2.88-2.82 (m, 2H), 2.73-2.63 (m, 6H), 2.10-2.08 (m, 2H), 1.93-1.75 (m, 5H), 1.47-1.25 (m, 1H); LC-MS (M+H)⁺: 493.2.

### Example 20: (S)-3-(3,5-dichlorophenyl)-3-((R)-1-(3-(5,6,7,8-tetrahydro-1,8-naphthyridin-2-yl)propyl)piperidine-3-carboxamido)propanoic acid (220)

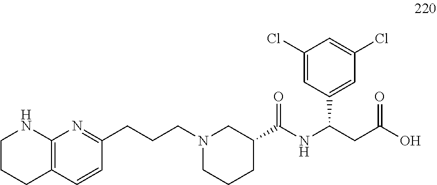 [SMILES: O=C(O)C[C@H](NC(=O)[C@@H]1CCCN(CCCc2ccc3c(n2)NCCC3)C1)c1cc(Cl)cc(Cl)c1]

Following the procedure illustrated in FIG. 2, Scheme 2, yielded compound 220. ¹H NMR (400 MHz, CDCl₃) δ 10.60 (br s, 1H), 9.93 (br s, 1H), 7.28 (d, J=1.6 Hz, 2H), 7.19 (d, J=6.0 Hz, 1H), 7.13 (d, J=2.0 Hz, 1H), 6.25 (d, J=7.6 Hz, 1H), 5.43 (br s, 1H), 3.41 (t, J=5.6 Hz, 2H), 2.99-2.95 (m, 3H), 2.83 (t, J=4.4 Hz, 2H), 2.69 (t, J=6.0 Hz, 3H), 2.63-2.32 (m, 5H), 1.97-1.84 (m, 8H); LC-MS (M+H)⁺: 519.3.

### Example 21: (S)-3-(6-methoxypyridin-3-yl)-3-((R)-1-(3-(5,6,7,8-tetrahydro-1,8-naphthyridin-2-yl)propyl)piperidine-3-carboxamido)propanoic acid hydrochloride (221)

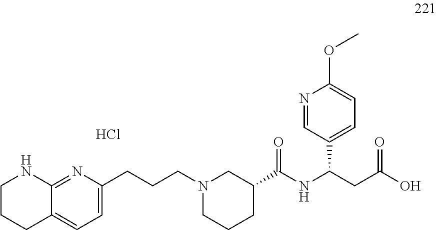 [SMILES: COc1ccc([C@H](CC(=O)O)NC(=O)[C@@H]2CCCN(CCCc3ccc4c(n3)NCCC4)C2)cn1 ; Cl]

Following the procedure illustrated in FIG. 2, Scheme 2, yielded compound 221. ¹H NMR (400 MHz, DMSO-d₆) δ 8.57 (br s, 1H), 8.10-8.05 (m, 1H), 7.67-7.61 (m, 1H), 7.07-7.04 (m, 1H), 6.76-6.75 (m, 1H), 6.27 (d, J=6.8 Hz, 1H), 5.22-5.09 (m, 1H), 3.81 (s. 3H), 3.22 (br s, 3H), 2.61-2.58 (m, 6H), 2.32-2.26 (m, 7H), 2.05-1.98 (m, 1H), 1.74-1.38 (m, 7H): LC-MS (M+H)⁺; 481.2.

### Example 22: 3-(3-chloro-5-(trifluoromethyl)phenyl)-3-((R)-1-(3-(5,6,7,8-tetrahydro-1,8-naphthyridin-2-yl) propyl)piperidine-3-carboxamido)propanoic acid hydrochloride (222)

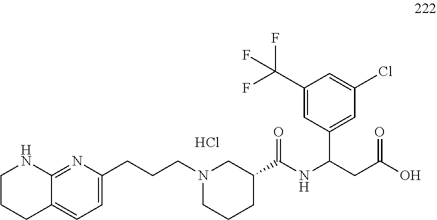 [SMILES: O=C(O)CC(NC(=O)[C@@H]1CCCN(CCCc2ccc3c(n2)NCCC3)C1)c1cc(Cl)cc(C(F)(F)F)c1 ; Cl]

Following the procedure illustrated in FIG. 2, Scheme 2, yielded compound 222. ¹H NMR (400 MHz, DMSO-dc) δ 14.11 (br s, 1H), 10.90-10.67 (m, 11H), 8.94-8.89 (m, 1H), 8.00 (br s, 1H), 7.75-7.60 (M, 4H), 6.66 (t, J=8.0 Hz, 1H), 5.22-5.16 (m, 1H), 3.45-3.44 (m, 2H), 3.04 (br s, 3H), 2.90-2.89 (m, 2H), 2.75-2.73 (m, 7H), 2.52-2.51 (m, 1H), 2.12-2.11 (m, 2H), 1.94-1.81 (m, 5H), 1.46-1.34 (m, 1H); LC-MS (M+H)⁺: 553.3.

### Example 23: 3-(3,5-bis(trifluoromethyl)phenyl)-3-((R)-1-(3-(5,6,7,8-tetrahydro-1,8-naphthyridin-2-yl)prop yl)piperidine-3-carboxamido)propanoic acid formic acid (223)

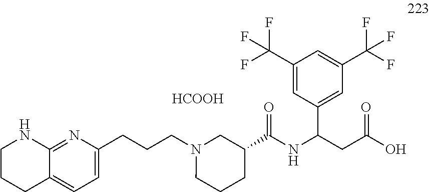 [SMILES: O=C(O)CC(NC(=O)[C@@H]1CCCN(CCCc2ccc3c(n2)NCCC3)C1)c1cc(C(F)(F)F)cc(C(F)(F)F)c1 ; "HCOOH"]

Following the procedure illustrated in FIG. 2, Scheme 2, yielded compound 223. ¹H NMR (400 MHz, DMSO-d₆) δ 8.91-8.89 (m, 1H), 8.19 (s, 1H), 8.11 (s, 1H), 8.00 (s, 1H), 7.95 (d, J=8.4 Hz, 1H), 7.15 (dd, J₁=35.6 Hz, J₂=7.2 Hz, 1H), 6.32 (dd, J₁=18.8 Hz, J₂=7.2 Hz, 1H), 5.38-5.26 (m, 1H), 4.57-4.43 (m, 2H), 3.23 (t, J=5.2 Hz, 2H), 2.75-2.59 (m, 7H), 2.47-2.42 (m, 4H), 1.74-1.37 (m, 8H); LC-MS (M+H)⁺: 587.7.

The following compounds, set forth in Table 2, were prepared according to the general procedure illustrated in Scheme 2, or analogous procedures thereto:

TABLE 2

| Compound | |
|---|---|
| No. | ¹H NMR Data |
| 328 | (400 MHz, DMSO-d₆) δ 8.52 (d, J = 7.6 Hz, 1H), 7.11-7.07 (m, 2H), |
| | 7.02-7.00 (m, 1H), 6.68 (s, 1H), 6.29 (d, J = 7.2 Hz, 1H), 5.11 (q, J = |
| | 8.0 Hz, 1H), 3.23-3.21 (m, 2H), 2.91 (t, J = 7.6 Hz, 2H), 2.62-2.57 |
| | (m, 4H), 2.43-2.26 (m, 6H), 2.12 (s, 1H), 2.05 (t, J = 7.6 Hz, 2H), |
| | 1.75-1.68 (m, 4H), 1.57-1.41 (m, 5H), 1.24 (s, 1H), 0.92-0.89 (m, |
| | 2H), 0.78-0.77 (m, 2H). |
| 345 | (400 MHz, DMSO-d₆) δ 14.18 (d, J = 1.2 Hz, 1H), 11.05-10.85 (m, |
| | 1H), 8.82-8.70 (m, 1H), 8.10-7.94 (m, 1H), 7.67-7.57 (m, 1H), 7.30 |
| | (d, J = 6.0 Hz, 1H), 7.18-7.10 (m, 2H), 6.97 (d, J = 6.8 Hz, 1H), 6.72- |
| | 6.63 (m, 1H), 5.80-5.68 (m, 1H), 2.87 (s, 5H), 2.74 (d, J = 6.0 Hz, |
| | 5H), 2.67-2.61 (m, 2H), 2.40-2.29 (m, 1H), 2.21-2.11 (m, 2H), 2.11- |
| | 1.97 (m, 2H), 1.97-1.87 (m, 2H), 1.87-1.67 (m, 4H), 1.34-1.21 (m, |
| | 1H), 0.93-0.84 (m, 2H), 0.81-0.69 (m, 1H), 0.64-0.53 (m, 1H). |
| 374 | (400 MHz, CDCl₃) δ 10.77 (s, 1H), 9.58 (d, J = 9.2 Hz, 1H), 9.19 (s, |
| | 1H), 8.11-7.91 (m, 2H), 7.74-7.63 (m, 2H), 7.15 (d, J = 7.2 Hz, 1H), |
| | 6.20 (d, J = 7.2 Hz, 1H), 5.85-5.68 (m, 1H), 5.07-4.81 (m, 1H), 3.41- |
| | 3.34 (m, 3H), 3.16-3.04 (m, 2H), 3.03-2.99 (m, 1H), 2.74-2.69 (m, |
| | 1H), 2.63 (t, J = 6.0 Hz, 2H), 2.47-2.35 (m, 3H), 2.27 (d, J = 13.6 Hz, |
| | 1H), 2.16 (dd, J₁ = 12.4 Hz, J₂ = 3.2 Hz, 1H), 2.06-1.97 (m, 2H), 1.85- |
| | 1.78 (m, 3H), 1.62-1.50 (m, 2H), 1.29-1.22 (m, 1H), 0.96-0.84 (m, 1H). |
| 377 | (400 MHz, DMSO-d₆) δ 9.32 (br s, 1H), 8.47 (br t, J = 8.0 Hz, 1H), |
| | 7.10-7.01 (m, 2H), 6.76-6.63 (m, 3H), 6.61-6.56 (m, 1H), 6.29- |
| | 6.23 (m, 1H), 5.07 (q, J = 6.8 Hz, 1H), 3.23 (br s, 3H), 2.77-2.66 (m, |
| | 1H), 2.61-2.57 (m, 4H), 2.46-2.40 (m, 2H), 2.36-2.31 (m, 1H), 2.26 |
| | (t, J = 6.0 Hz, 2H), 2.14-1.94 (m, 2H), 1.76-1.58 (m, 6H), 1.46-1.30 |
| | (m, 2H). |
| 412 | (400 MHz, DMSO-d₆) δ 8.50 (d, J = 8.4 Hz, 1H), 7.21 (t, J = 8.0 Hz, |
| | 1H), 7.06 (d, J = 7.6 Hz, 1H), 6.90 (d, J = 7.6 Hz, 1H), 6.72 (s, 1H), |
| | 6.68 (br s, 1H), 6.60 (dd, J₁ = 8.0 Hz, J₂ = 2.0 Hz, 1H), 6.27 (d, J = 7.2 |
| | Hz, 1H), 5.26-5.19 (m, 1H), 5.13 (q, J = 7.2 Hz, 1H), 4.90 (t, J = 6.8 |
| | Hz, 2H), 4.55-4.48 (m, 2H), 3.23 (t, J = 5.2 Hz, 3H), 2.74 (m, J = 10.0 |
| | Hz, 1H), 2.62-2.59 (m, 4H), 2.44 (t, J = 7.6 Hz, 2H), 2.37-2.32 (m, |
| | 1H), 2.31-2.25 (m, 2H), 2.09 (t, J = 9.6 Hz, 1H), 1.99 (t, J = 9.2 Hz, |
| | 1H), 1.78-1.69 (m, 4H), 1.66-1.59 (m, 2H), 1.47-1.31 (m, 2H). |
| 413 | (400 MHz, DMSO-d₆) δ 8.89 (d, J = 2.0 Hz, 1H), 8.80 (d, J = 6.8 Hz, |
| | 1H), 8.19 (d, J = 2.0 Hz, 1H), 7.99 (d, J = 8.4 Hz, 1H), 7.93 (d, J = 8.0 |
| | Hz, 1H), 7.75-6.68 (m, 1H), 7.59 (t, J = 7.2 Hz, 1H), 7.06 (d, J = 7.2 |
| | Hz, 1H), 6.95-6.80 (m, 1H), 6.27 (d, J = 7.2 Hz, 1H), 5.36 (q, J = 7.6 |
| | Hz, 1H), 3.22 (t, J = 5.2 Hz, 2H), 2.82 (d, J = 6.8 Hz, 2H), 2.74 (d, J = |
| | 10.4 Hz, 1H), 2.63-2.53 (m, 3H), 2.46-2.38 (m, 3H), 2.35-2.25 (m, |
| | 2H), 2.23-2.14 (m, 1H), 2.07-1.97 (m, 1H), 1.80-1.57 (m, 6H), 1.50- |
| | 1.28 (m, 2H). |
| 535 | (400 MHz, DMSO-d₆) δ 8.52 (d, J = 8.4 Hz, 1H), 7.20 (t, J = 8.0 Hz, |
| | 1H), 7.06 (d, J = 7.2 Hz, 1H), 6.91 (d, J = 7.6 Hz, 1H), 6.82 (br s, 1H), |
| | 6.74 (s, 1H), 6.60 (dd, J₁ = 8.0 Hz, J₂ = 2.0 Hz, 1H), 6.27 (d, J = 7.2 |
| | Hz, 1H), 5.26-5.18 (m, 1H), 5.13 (q, J = 8.0 Hz, 1H), 4.90 (t, J = 6.8 |
| | Hz, 2H), 4.54-4.48 (m, 2H), 3.23 (br s, 3H), 2.97-2.86 (m, 1H), 2.68- |
| | 2.63 (m, 1H), 2.62-2.59 (m, 3H), 2.46-2.40 (m, 2H), 2.37-2.33 (m, |
| | 1H), 2.27 (t, J = 6.8 Hz, 2H), 2.17-2.10 (m, 1H), 2.06-1.98 (m, 1H), |
| | 1.77-1.69 (m, 4H), 1.67-1.57 (m, 2H), 1.47-1.38 (m, 2H) |
| 539 | (400 MHz, CDCl₃) δ 10.63-10.36 (m, 1H), 9.04-8.94 (m, 1H), 8.01 |
| | (t, J = 4.0 Hz, 2H), 7.74-7.58 (m, 2H), 7.13 (d, J = 7.2 Hz, 1H), 6.15 |
| | (d, J = 7.2 Hz, 1H), 5.80-5.78 (m, 1H), 3.75-3.63 (m, 1H), 3.40- |
| | 3.32 (m, 2H), 3.17-3.11 (m, 1H), 2.98-2.93 (m, 2H), 2.87-2.76 (m, |
| | 3H), 2.63 (t, J = 6.0 Hz, 2H), 2.53-2.37 (m, 4H), 2.07-2.01 (m, 1H), |
| | 1.96-1.95 (m, 1H), 1.84-1.81 (m, 2H), 1.69-1.65 (m, 2H), 1.26 (s, |
| | 1H), 0.89-0.87 (m, 1H). |
| 540 | (400 MHz, DMSO-d₆) δ 8.91 (d, J = 2.0 Hz, 1H), 8.79 (d, J = 7.6 Hz, |
| | 1H), 8.24 (d, J = 1.6 Hz, 1H), 7.99 (d, J = 8.4 Hz, 1H), 7.91 (d, J = 8.4 |
| | Hz, 1H), 7.76-6.68 (m, 1H), 7.59 (t, J = 6.8 Hz, 1H), 7.07 (d, J = 7.2 |
| | Hz, 1H), 6.27 (d, J = 7.6 Hz, 1H), 5.37 (q, J = 7.6 Hz, 1H), 3.21 (br s, |
| | 2H), 2.82 (d, J = 6.8 Hz, 2H), 2.76-2.64 (m, 2H), 2.58 (t, J = 6.0 Hz, |
| | 3H), 2.47-2.40 (m, 3H), 2.37-2.31 (m, 2H), 2.24-2.14 (m, 1H), 1.81- |
| | 1.67 (m, 4H), 1.65-1.40 (m, 4H). |
| 541 | (400 MHz, DMSO-d₆) δ 8.45 (d, J = 7.6 Hz, 1H), 7.09-7.04 (m, 2H), |
| | 7.00-6.98(m, 1H), 6.63 (s, 1H), 6.26 (d, J = 7.2 Hz, 1H), 5.10 (q, J = |
| | 7.6 Hz, 1H), 3.23 (t, J = 5.2 Hz, 2H), 2.91 (t, J = 7.6 Hz, 2H), 2.71- |
| | 2.67 (m, 1H), 2.62-2.56 (m, 5H), 2.44-2.42 (m, 2H), 2.34-2.33 (m, |
| | 1H), 2.29-2.25 (m, 2H), 2.05 (t, J = 7.6 Hz, 2H), 1.76-1.69 (m, 4H), |
| | 1.64-1.59 (m, 2H), 1.46-1.20 (m, 4H), 0.93-0.90 (m, 2H), 0.81- |
| | 0.79 (m, 2H). |
| 556 | (400 MHz, DMSO-d₆) δ 14.27-14.10 (m, 1H), 10.91-10.78 (m, 1H), |
| | 8.84-8.73 (m, 1H), 8.01 (s, 1H), 7.61 (d, J = 7.2 Hz, 1H), 7.39-7.30 |
| | (m, 1H), 7.20-7.10 (m, 2H), 6.98 (d, J = 7.2 Hz, 1H), 6.69-6.62 (m, |
| | 1H), 5.81-5.70 (m, 1H), 3.07-2.84 (m, 5H), 2.77-2.52 (m, 8H), 2.21- |
| | 1.89 (m, 6H), 1.89-1.73 (m, 4H), 1.52-1.37 (m, 1H), 0.95-0.86 (m, |
| | 2H), 0.77-0.70 (m, 1H), 0.62-0.54 (m, 1H). |

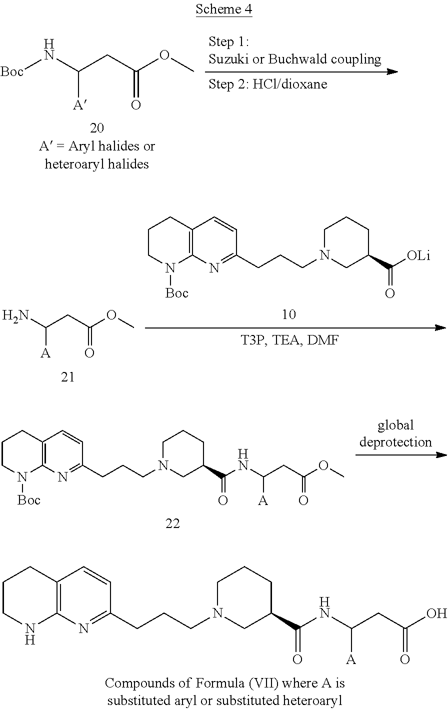

### FIG. 3

FIG. 3, Scheme 4, illustrates a general procedures for preparation of compounds of Formula (VII) where A is substituted aryl or substituted heteroaryl.

### General Procedure for Preparation of Compound 21

### Step 1

To a solution of compound 20 (1.00 eq), boronic acid/ester (1.50 eq), K₂CO₃ (3.00 eq) in dioxane and H₂O was added Pd(dppf)Cl₂ (0.100 eq) under N₂. The mixture was stirred at 80° C. for 5 hrs, quenched with water and extracted with ethyl acetate. The combined organic extracts were dried over Na₂SO₄, concentrated to give a residue which was purified by column chromatography to yield the Boc protected product of the transition metal mediated cross coupling.

### Step 2

To a solution of Boc protected product of the cross-coupling reaction (1.00 eq) from step 1 in DCM was added HCl/dioxane (5.00 eq) at 0° C. The mixture was stirred at 25° C. for 2 hrs and concentrated to give crude compound 21.

### General Procedure for Preparation of Compound 22

To a solution of compound 21 (1.20 eq) and compound 10 (1.00 eq) in DCM was added T3P (2.00 eq) and DIEA (2.00 eq). The mixture was stirred at 25° C. for 2 hrs, diluted with water and extracted with EtOAc. The combined organic extracts were washed with brine, dried over Na₂SO₄, filtered and concentrated to give a residue which was purified by prep-HPLC (basic condition, column: Waters Xbridge 150*25 mm*5 um; mobile phase: [water (0.05% ammonia hydroxide v/v)−ACN]) to provide compound 22.

### General Procedure for Preparation of Compounds of Formula (VII)

To a solution of compound 22 (1.00 eq) in H₂O was added HCl/dioxane (100 eq). The mixture was stirred at 60° C. for 3 hrs, concentrated to give a residue which was purified by prep-HPLC (HCl condition; column: Phenomenex luna C18 150*25 mm*10 um; mobile phase: [water (0.05% HCl)−ACN] to provide a compound of Formula (VII). SFC was used to separated isomers when necessary.

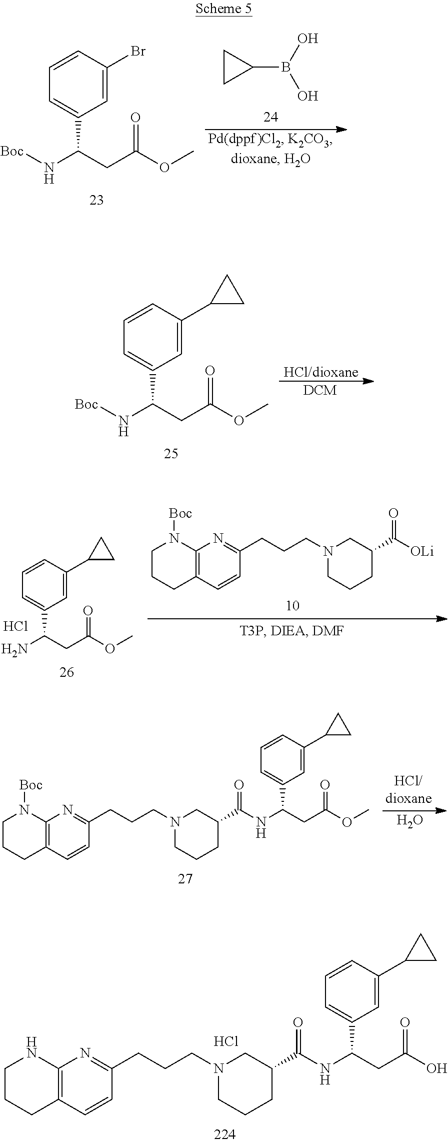

Scheme 5 illustrates the synthesis of compound 224 and exemplifies the preparation of compounds of Formula (VII) as shown in FIG. 3, Scheme 4.

### Preparation of Methyl (S)-3-((Tert-Butoxycarbonyl)Amino)-3-(3-Cyclopropylphenyl)Propanoate (25)

To a solution of methyl (S)-3-(3-bromophenyl)-3-((tert-butoxycarbonyl)amino)propanoate (23) (0.500 g, 1.40 mmol, 1.00 eq) and cyclopropylboronic acid (24) (180 mg, 2.09 mmol, 1.50 eq), K₂CO₃ (579 mg, 4.19 mmol, 3.00 eq) in dioxane (8.00 mL) and H₂O (2.00 mL) was added Pd(dppf)Cl₂ (102 mg, 139 umol, 0.100 eq) under N₂ and the mixture stirred at 80° C. for 5 hrs until TLC (Petroleum ether. Ethyl acetate=10:1, R_{f}=0.38) indicated compound 23 was completely consumed and several new spots with larger polarity were detected. The reaction mixture was quenched with water (15.0 mL) and extracted with ethyl acetate 30.0 mL (10.0 mL*3). The combined organic extracts were dried over Na₂SO₄ and concentrated under vacuum to give a residue which was purified by column chromatography (SiO₂, Petroleum ether: Ethyl acetate=10:1 to 20:1) to yield compound 25 (320 mg, 956 umol, 68.5% yield, 95.4% purity) as a colorless oil. ¹H NMR (400 MHz, CDCl₃) δ 7.21 (t, J=7.6 Hz, 1H), 7.06 (d, J=7.6 Hz, 1H), 7.01 (s, 1H), 6.95 (d, J=7.6 Hz, 1H), 5.40 (br s, 1H), 5.06 (br s, 1H), 3.63 (s, 3H), 2.86-2.78 (m, 2H), 1.92-1.87 (m, 1H), 1.43 (s, 9H), 0.97-0.93 (m, 2H)), 0.70-0.67 (m, 2H); LC-MS (M+H)⁺: 320.2; SFC chiral purity: 99.4%.

### Preparation of Methyl (S)-3-Amino-3-(3-Cyclopropylphenyl)Propanoate Hydrochloride (26)

To a solution of compound 25 (0.300 g, 939 umol, 1.00 eq) in DCM (3.00 mL) was added HCl/dioxane (4.00 M, 1.17 mL, 5.00 eq) at 0° C. The mixture was stirred at 25° C. for 2 hrs until LC-MS showed compound 25 was completely consumed and detected one main peak with the correct mass. The reaction mixture was concentrated to give compound 26 (240 mg, 938 umol, 99.9% yield, HCl) as a yellow solid. ¹H NMR (400 MHz, DMSO-d₆) δ 8.68 (s. 3H), 7.28-7.22 (m, 3H), 7.11-7.08 (m, 1H), 4.53 (d, J=3.2 Hz, 1H), 3.56 (s, 3H), 3.47-3.36 (m, 2H), 1.95-1.87 (m, 1H), 0.99-0.94 (m, 2H), 0.73-0.69 (m, 2H).

### Preparation of tert-Butyl 7-(3-((R)-3-(((S)-1-(3-Cyclopropylphenyl)-3-Methoxy-3-Oxopropyl)Carbamoyl)Piperidin-1-Yl)Propyl)-3,4-Dihydro-1,8-Naphthyridine-1(2H)-Carboxylate (27)

To a solution of compound 26 (59.8 mg, 234 umol, 1.20 eq, HCl) and compound 10 (80.0 mg, 195 umol, 1.00 eq, Li) in DCM (1.00 mL) was added T3P (124 mg, 390 umol, 116 uL, 2.00 eq) and DIEA (50.4 mg, 390 umol, 67.9 uL, 2.00 eq). The mixture was stirred at 25° C. for 2 hrs until LC-MS detected ˜50% of the desired product, diluted with water (2.00 mL) and extracted with EtOAc (3.00 mL*3). The combined organic extracts were washed with brine (3.00 mL), dried over Na₂SO₄, filtered and concentrated under reduced pressure to give a residue. The crude product was combined with a similar reaction run on a 10.0 mg scale and the combined residue was purified by prep-HPLC (basic condition, column: Waters Xbridge 150*25 mm*5 um; mobile phase: [water (0.05% ammonia hydroxide v/v)−ACN]: B %: 48%-78%, 10 min) to yield compound 27 (38.0 mg, 54.0 umol, 13.8% yield, 85.9% purity) as a colorless oil. LC-MS (M+H)⁺: 605.5; SFC chiral purity: 100%.

### Example 24: (S)-3-(3-Cyclopropylphenyl)-3-((R)-1-(3-(5,6,7,8-Tetrahydro-1,8-Naphthyridin-2-YI)Propyl)Piperidine-3-Carboxamido)Propanoic Acid hydrochloride (224)

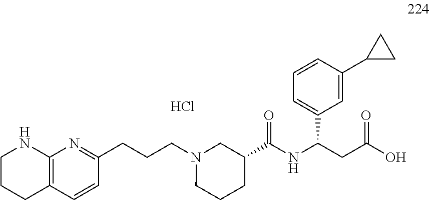 [SMILES: O=C(O)C[C@H](NC(=O)[C@@H]1CCCN(CCCc2ccc3c(n2)NCCC3)C1)c1cccc(C2CC2)c1 ; Cl]

To a solution of compound 27 (28.0 mg, 46.3 umol, 1.00 eq) in H₂O (1.00 mL) was added HCl/dioxane (4.00 M, 1.07 mL, 100 eq). The mixture was stirred at 60° C. for 3 hrs until LC-MS showed complete consumption of compound 27 and the detection of one main peak with the correct mass. The reaction mixture was concentrated under reduced pressure to give a residue, which was purified by prep-HPLC (HCl condition; column: Phenomenex luna C18 150*25 mm*10 um; mobile phase: [water (0.05% HCl)−ACN]; B %: 11%-41%, 10 min) to yield compound 224 (23.7 mg, 44.9 umol, 97.1% yield, 97.7% purity. HCl) as a white solid. LC-MS (M+H)⁺: 491.1: ¹H NMR (400 MHz, DMSO-d₆) δ 8.72 (s. 1H). 7.91 (s. 1H), 7.60 (d, J=7.6 Hz, 1H), 7.17 (d, J=7.6 Hz, 1H), 7.04 (d, J=7.6 Hz, 1H), 6.99 (s, 1H), 6.92 (d, J=7.6 Hz. 1H), 6.65 (d, J=7.2 Hz, 1H), 5.11 (q, J=7.6 Hz, 1H), 3.21-3.16 (m, 2H), 3.10-3.02 (m, 3H), 2.93-2.86 (m, 2H), 2.76-2.54 (m, 8H), 2.10 (s, 2H), 1.92-1.76 (m, 6H), 1.41-1.28 (m, 1H), 0.97-0.90 (m, 2H), 0.64-0.60 (m, 2H); HPLC purity: 97.7% (215 nm); SFC chiral purity: 100%.

### Example 25: Preparation of

(S)-3-(3-(3,5-dimethyl-1H-pyrazol-1-yl)phenyl)-3-((R)-1-(3-(5,6,7,8-tetrahydro-1,8-naphthyridin-2-yl)propyl)piperidine-3-carboxamido)propanoic acid hydrochloride (225)

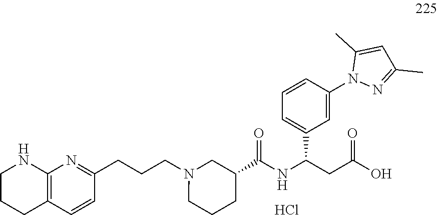 [SMILES: Cc1cc(C)n(-c2cccc([C@H](CC(=O)O)NC(=O)[C@@H]3CCCN(CCCc4ccc5c(n4)NCCC5)C3)c2)n1 ; Cl]

Following the procedure illustrated in FIG. 3, Scheme 4, yielded compound 225. LC-MS (M+H)⁺: 545.4; ¹H NMR (400 MHz, DMSO-d₆) δ 14.44 (brs, 1H), 11.20 (brs, 1H), 8.91 (d, J=8.0 Hz, 1H), 8.12 (brs, 1H), 7.61 (d, J=3.2 Hz, 1H), 7.46-7.40 (m, 2H), 7.36-7.30 (m, 2H), 6.68 (d, J=7.2 Hz, 1H), 6.10 (s, 1H), 5.23-5.17 (m, 1H), 3.42-3.38 (m, 4H), 3.04-2.89 (m, 4H), 2.83-2.67 (m, 7H), 2.54 (m, 1H), 2.28 (s, 3H), 2.18-2.12 (m, 5H), 2.00-1.90 (m, 2H), 1.82-1.77 (m, 3H); HPLC purity: 92.6% (254 nm): SFC chiral purity: 100%.

### Example 26: Preparation of

(S)-3-(3-(1,4-dimethyl-1H-pyrazol-5-yl)phenyl)-3-((R)-1-(3-(5,6,7,8-tetrahydro-1,8-naphthyridin-2-yl)propyl)piperidine-3-carboxamido)propanoic acid hydrochloride (226)

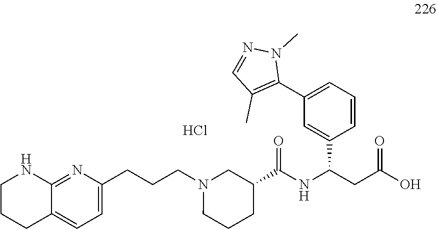 [SMILES: Cc1cnn(C)c1-c1cccc([C@H](CC(=O)O)NC(=O)[C@@H]2CCCN(CCCc3ccc4c(n3)NCCC4)C2)c1 ; Cl]

Following the procedure illustrated in FIG. 3, Scheme 4, yielded compound 226. LC-MS (M+H)⁺: 545.3; ¹H NMR (400 MHz, DMSO-d₆) δ 14.32 (s, 1H), 11.08 (s, 1H), 8.88 (d, J=8.0 Hz, 1H), 8.08 (s, 1H), 7.62 (d, J=7.2 Hz, 1H), 7.48-7.44 (m, 1H), 7.38-7.37 (m, 2H), 7.34 (s, 1H), 7.29-7.27 (m, 1H), 6.68 (d, J=7.6 Hz, 1H), 5.22 (q, J=7.6 Hz, 1H), 3.71 (s, 3H), 3.42-3.40 (m, 4H), 3.05-3.04 (m, 2H), 2.97-2.90 (m, 2H), 2.76-2.73 (m, 7H), 2.19-2.12 (m, 2H), 1.96 (s, 3H), 1.92-1.76 (m, 5H), 1.34-1.31 (m, 1H): HPLC purity: 93.7%, (220 nm); SFC chiral purity: 100%.

### Example 27: Preparation of

(S)-3-(3-(pyrrolidin-1-yl)phenyl)-3-((R)-1-(3-(5,6,7,8-tetrahydro-1,8-naphthyridin-2-yl)propyl)piperidine-3-carboxamido)propanoic acid (227)

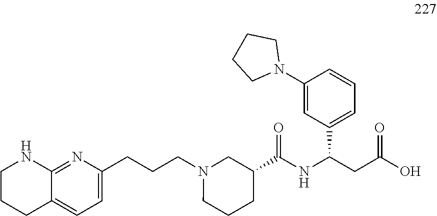 [SMILES: O=C(O)C[C@H](NC(=O)[C@@H]1CCCN(CCCc2ccc3c(n2)NCCC3)C1)c1cccc(N2CCCC2)c1]

Following the procedure illustrated in FIG. 3, Scheme 4, yielded compound 227. ¹H NMR (400 MHz, DMSO-d₆) δ 8.59 (s, 1H), 7.09-7.04 (m, 2H), 6.51 (d, J=7.6 Hz, 1H), 6.45 (s, 1H), 6.37 (dd, J=8.0 Hz, J=2.0 Hz, 1H). 6.28 (d, J=7.6 Hz, 1H), 5.13-5.07 (m, 1H), 3.23 (t, J=4.8 Hz, 4H), 3.18 (t, J=7.6 Hz, 4H), 2.67-2.66 (m, 1H), 2.63-2.59 (m, 4H), 2.52-2.51 (m, 2H), 2.47-2.45 (m, 4H), 1.94-1.92 (m, 6H), 1.76-1.70 (m, 4H), 1.44-1.14 (m, 2H); LC-MS (M+H)⁺: 520.4: HPLC purity: 97.5% (220 nm); SFC chiral purity: 100%.

### Example 28: Preparation of

(S)-3-(3-(4-methylpiperazin-1-yl)phenyl)-3-((R)-1-(3-(5,6,7,8-tetrahydro-1,8-naphthyridin-2-yl)propyl)piperidine-3-carboxamido)propanoic acid (228)

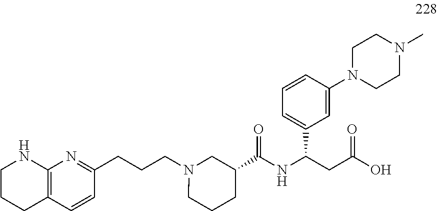 [SMILES: CN1CCN(c2cccc([C@H](CC(=O)O)NC(=O)[C@@H]3CCCN(CCCc4ccc5c(n4)NCCC5)C3)c2)CC1]

Following the procedure illustrated in FIG. 3, Scheme 4, yielded compound 228. ¹H NMR (400 MHz, DMSO-d₆) δ 10.66 (br s, 1H), 9.79 (br s, 1H), 7.18-7.11 (m, 2H), 7.01 (s, 1H), 6.93 (d, J=7.6 Hz, 1H), 6.70 (dd, J₁=8.0 Hz, J₂=2.0 Hz, 1H), 6.19 (d, J=7.2 Hz, 1H), 5.47-5.45 (m, 1H), 3.40-3.39 (m, 2H), 3.17 (t, J=4.4 Hz, 4H), 2.89-2.80 (m, 5H), 2.67-2.60 (m, 4H), 2.56 (t, J=4.8 Hz, 4H), 2.52-2.37 (m, 3H), 2.35 (s, 3H), 2.33-2.28 (m, 1H), 1.88-1.79 (m, 6H), 1.59-1.56 (m, 2H): LC-MS (M+H)⁺: 549.5: HPLC purity: 96.6%. (220 nm); SFC chiral purity: 100%.

### Example 29: (S)-3-(3-morpholinophenyl)-3-((R)-1-(3-(5,6,7,8-tetrahydro-1,8-naphthyridin-2-yl)propyl)piperidine-3-carboxamido)propanoic acid hydrochloride (229)

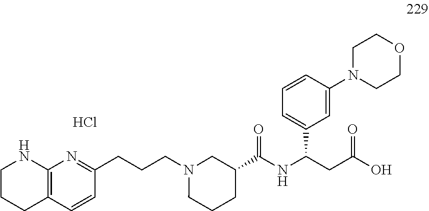 [SMILES: O=C(O)C[C@H](NC(=O)[C@@H]1CCCN(CCCc2ccc3c(n2)NCCC3)C1)c1cccc(N2CCOCC2)c1 ; Cl]

Following the procedure illustrated in FIG. 3, Scheme 4, yielded compound 229. ¹H NMR (400 MHz, DMSO-d₆) δ 14.22 (s, 1H), 11.05 (s, 1H), 8.73 (d, J=8.4 Hz, 1H), 8.04 (s, 1H), 7.62 (d, J=7.2 Hz, 1H), 7.18 (t, J=8.0 Hz, 1H), 6.98 (s, 1H), 6.89 (d, J=8.0 Hz, 1H), 6.80 (d, J=7.6 Hz, 1H), 6.68 (d, J=7.2 Hz, 1H), 5.15-5.09 (m, 1H), 3.76 (t, J=4.8 Hz, 4H), 3.12 (s, 4H), 3.04-2.91 (m, 6H), 2.79-2.63 (m, 9H), 2.16-2.14 (m, 2H), 1.93-1.80 (m, 6H): LC-MS (M+H)⁺: 536.6.

### Example 30: (S)-3-(3-(3,3-dimethylpiperidin-1-yl)phenyl)-3-((R)-1-(3-(5,6,7,8-tetrahydro-1,8-naphthyridin-2-yl)propyl)piperidine-3-carboxamido)propanoic acid (230)

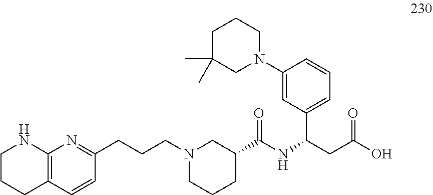 [SMILES: CC1(C)CCCN(c2cccc([C@H](CC(=O)O)NC(=O)[C@@H]3CCCN(CCCc4ccc5c(n4)NCCC5)C3)c2)C1]

Following the procedure illustrated in FIG. 3, Scheme 4, yielded compound 230. ¹H NMR (400 MHz, CDCl₃) δ 10.70 (br s, 1H), 9.79 (br s, 1H), 7.17 (d, J=7.2 Hz, 1H), 7.10 (t, J=7.6 Hz, 1H), 6.97 (s, 1H), 6.86 (d, J=7.6 Hz, 1H), 6.68 (dd, J₁=8.0 Hz, J₂=2.0 Hz, 1H), 6.19 (d, J=7.2 Hz, 1H), 5.48-5.45 (m, 1H), 3.40-3.39 (m, 2H), 3.02-2.99 (m, 2H), 2.89-2.85 (m, 5H), 2.73 (d, J=4.4 Hz, 2H), 2.67-2.40 (m, 7H), 2.34-2.29 (m, 1H), 1.88-1.80 (m, 6H), 1.78-1.64 (m, 4H), 1.32-1.29 (m, 2H), 0.96 (s, 6H); LC-MS (M+H)⁺: 562.2.

### Example 31: (S)-3-(3-cyclopentylphenyl)-3-((R)-1-(3-(5,6,7,8-tetrahydro-1,8-naphthyridin-2-yl)propyl)piperidine-3-carboxamido)propanoic acid hydrochloride (231)

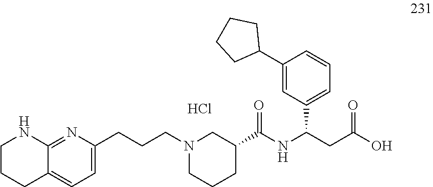 [SMILES: O=C(O)C[C@H](NC(=O)[C@@H]1CCCN(CCCc2ccc3c(n2)NCCC3)C1)c1cccc(C2CCCC2)c1 ; Cl]

Following the procedure illustrated in FIG. 3, Scheme 4, yielded compound 231. ¹H NMR (400 MHz, DMSO-d₆) δ 14.4-14.1 (m, 1H), 11.2-10.9 (m, 1H), 8.77 (d, J=7.2 Hz, 1H), 8.06 (s, 1H), 7.26 (d, J=7.2 Hz, 1H), 7.23-7.15 (m, 2H), 7.12-7.06 (m, 2H), 6.67 (d, J=7.2 Hz, 1H), 5.20-5.07 (m, 1H), 3.46-3.42 (m, 3H), 3.13-2.84 (m, 6H), 2.83-2.60 (m, 7H), 2.21-2.10 (m, 2H), 2.04-1.88 (m, 4H), 1.86-1.70 (m, 5H), 1.68-1.58 (m, 2H), 1.56-1.44 (m, 2H), 1.43-1.25 (m, 1H); LC-MS (M+H)⁺: 519.4.

### Example 32: (S)-3-(3-(piperidin-1-yl)phenyl)-3-((R)-1-(3-(5,6,7,8-tetrahydro-1,8-naphthyridin-2-yl)prop yl)piperidine-3-carboxamido)propanoic acid hydrochloride (232)

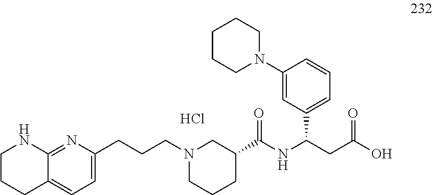 [SMILES: O=C(O)C[C@H](NC(=O)[C@@H]1CCCN(CCCc2ccc3c(n2)NCCC3)C1)c1cccc(N2CCCCC2)c1 ; Cl]

Following the procedure illustrated in FIG. 3, Scheme 4, yielded compound 232. ¹H NMR (400 MHz, DMSO-d₆) δ 14.3-14.1 (m, 1H), 11.1-10.8 (m, 1H)), 8.86 (d, J=8.0 Hz, 1H), 8.67 (s, 1H), 8.06 (s, 1H), 7.77 (s, 1H), 7.62 (d, J=7.2 Hz, 1H), 7.46 (t, J=7.6 Hz, 1H), 7.40-7.30 (m. 1H), 6.68 (d, J=7.2 Hz, 1H), 5.25-5.10 (m, 1H), 4.62-4.47 (m, 11H), 3.40-3.34 (m, 8H), 3.12-3.01 (m, 3H), 3.00-2.87 (m, 3H), 2.82-2.65 (m, 6H), 2.22-2.09 (m, 2H), 2.03-1.77 (m, 9H), 1.68-1.59 (m, 2H), 1.40-1.22 (m, 1H): LC-MS (M+H)⁺: 534.4.

### Example 33: 3-(1-phenyl-1H-pyrazol-4-yl)-3-((R)-1-(3-(5,6,7,8-tetrahydro-1,8-naphthyridin-2-yl)propyl) piperidine-3-carboxamido)propanoic acid hydrochloride (233)

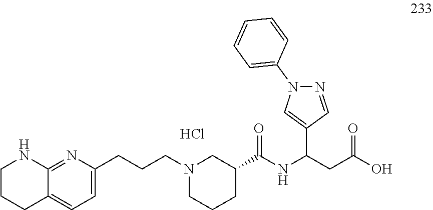 [SMILES: O=C(O)CC(NC(=O)[C@@H]1CCCN(CCCc2ccc3c(n2)NCCC3)C1)c1cnn(-c2ccccc2)c1 ; Cl]

Following the procedure illustrated in FIG. 3, Scheme 4, yielded compound 233. ¹H NMR (400 MHz, DMSO-d₆) δ 14.24 (br s, 1H), 10.92-10.82 (m, 1H), 8.66 (d, J=6.4 Hz, 1H), 8.38 (d, J=12.0 Hz, 1H), 8.06 (br s, 1H), 7.77 (dd. J₁=7.2 Hz, J₂=5.6 Hz, 2H), 7.63-7.50 (m, 2H), 7.48 (t, J=8.0 Hz, 2H), 7.29 (m, 1H), 6.67 (t, J=8.0 Hz, 1H), 5.21 (dd, J=7.2 Hz. J₂=4.4 Hz, 1H), 3.29 (m, 2H), 3.06 (br s, 2H), 2.79 (m, 10H), 2.14 (s. 2H), 1.91-1.80 (m, 5H), 1.44-1.41 (m, 1H); LC-MS (M+H)⁺: 517.3.

### Example 34: (S)-3-(3-(2,2-dimethylmorpholino)phenyl)-3-((R)-1-(3-(5,6,7,8-tetrahydro-1,8-naphthyridin-2-yl)propyl)piperidine-3-carboxamido)propanoic acid (234)

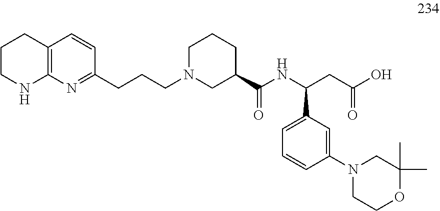 [SMILES: CC1(C)CN(c2cccc([C@H](CC(=O)O)NC(=O)[C@@H]3CCCN(CCCc4ccc5c(n4)NCCC5)C3)c2)CCO1]

Following the procedure illustrated in FIG. 3, Scheme 4, yielded compound 234. ¹H NMR (400 MHz, DMSO-d₆) δ 8.50 (d, J=8.0 Hz, 1H), 7.12 (t, J=8.0 Hz, 1H), 7.05 (d, J=7.6 Hz, 1H), 6.85 (s, 1H), 6.78-6.64 (m, 2H), 6.264 (s, 1H), 6.26 (d, J=7.2 Hz, 1H), 5.14-5.06 (m, 1H), 3.72 (t, J₁=4.2 Hz, 2H), 3.23 (t, J=4.2 Hz, 2H), 3.00 (t, J=4.2 Hz, 2H), 2.89 (s, 2H), 2.73-2.70 (m, 1H), 2.62-2.58 (m, 5H), 2.45-2.41 (m, 2H), 2.38-2.36 (m, 1H), 2.29-2.25 (m, 2H), 2.12-2.07 (m, 2H), 1.77-1.67 (m, 4H), 1.64-1.61 (m, 2H), 1.47-1.29 (m, 2H), 1.23-1.21 (m, 6H); LC-MS (M+H)⁺: 564.5.

### Example 35: (S)-3-(3-(3,3-dimethylmorpholino)phenyl)-3-((R)-1-(3-(5,6,7,8-tetrahydro-1,8-naphthyridin-2-yl)propyl)piperidine-3-carboxamido)propanoic acid (235)

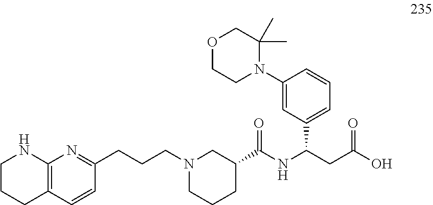 [SMILES: CC1(C)COCCN1c1cccc([C@H](CC(=O)O)NC(=O)[C@@H]2CCCN(CCCc3ccc4c(n3)NCCC4)C2)c1]

Following the procedure illustrated in FIG. 3, Scheme 4, yielded compound 235. ¹H NMR (400 MHz, DMSO-d₆) δ 8.54 (d, J=8.4 Hz, 1H), 7.19 (t, J=7.6 Hz, 1H), 7.07-6.97 (m, 4H), 6.71 (br s, 1H), 627 (d, J=7.2 Hz, 1H), 5.16-5.11 (m, 1H), 3.68 (t, J=4.8 Hz, 2H), 3.38 (s, 2H), 3.23 (t, J=5.2 Hz, 2H), 3.00-2.99 (m, 2H), 2.72 (d, J=9.6 Hz, 1H), 2.62-2.59 (m, 5H), 2.44 (t, J=8.0 Hz, 2H), 2.38-2.33 (M. 1H), 2.28-2.26 (m, 2H), 2.12-2.07 (m, 1H), 2.02-1.97 (m, 1H), 1.75-1.61 (m, 6H), 1.44-1.36 (m, 2H), 0.92 (d, J=2.0 Hz, 6H), LC-MS (M+H)⁺: 564.1.

### Example 36: (S)-3-(3-((2S,6R)-2,6-dimethylmorpholino)phenyl)-3-((R)-1-(3-(5,6,7,8-tetrahydro-1,8-naphthyridin-2-yl)propyl)piperidine-3-carboxamido)propanoic acid (236)

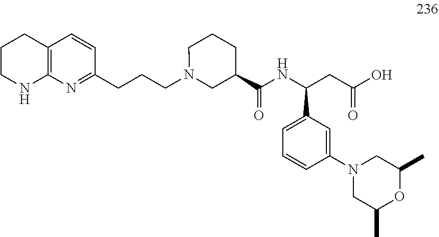 [SMILES: C[C@@H]1CN(c2cccc([C@H](CC(=O)O)NC(=O)[C@@H]3CCCN(CCCc4ccc5c(n4)NCCC5)C3)c2)C[C@H](C)O1]

Following the procedure illustrated in FIG. 3, Scheme 4, yielded compound 236. ¹H NMR (400 MHz, CDCl₃) δ 10.71 (s, 1H), 9.82 (br s, 1H), 7.19-7.12 (m, 2H), 6.98-6.95 (m, 2H), 6.70 (dd, J₁=8.0 Hz, J₂=6.0 Hz, 1H), 6.20 (d, J=7.2 Hz, 1H), 5.49-5.46 (m, 1H), 3.78-3.74 (m, 2H), 3.41-3.39 (m, 4H), 2.88-2.81 (m, 5H), 2.68-2.31 (m, 10H), 1.88-1.77 (m, 6H), 1.59-1.55 (m, 2H), 1.21 (dd, J₁=6.4 Hz, J₂=4.4 Hz, 6H): LC-MS (M+H)⁺: 564.1.

### Example 37: (S)-3-(3-(4,4-dimethylpiperidin-1-yl)phenyl)-3-((R)-1-(3-(5,6,7,8-tetrahydro-1,8-naphthyridin-2-yl)propyl)piperidine-3-carboxamido)propanoic acid (237)

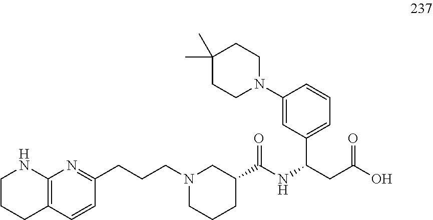 [SMILES: CC1(C)CCN(c2cccc([C@H](CC(=O)O)NC(=O)[C@@H]3CCCN(CCCc4ccc5c(n4)NCCC5)C3)c2)CC1]

Following the procedure illustrated in FIG. 3, Scheme 4, yielded compound 237. ¹H NMR (400 MHz, CDCl₃) δ 10.69 (s, 1H), 9.80 (s, 1H), 7.17 (d, J=7.6 Hz, 1H), 7.12 (t, J=7.6 Hz, 1H), 7.02 (s, 1H), 6.89 (d, J=7.2 Hz, 1H), 6.73 (d, J=8.0 Hz, 1H), 6.19 (d, J=7.2 Hz, 1H), 5.49-5.47 (m, 1H), 3.40-3.39 (m, 2H), 3.09 (t, J=5.6 Hz, 4H), 2.89-2.85 (m, 5H), 2.67-2.63 (m, 3H), 2.54 (br s, 1H), 2.44-2.29 (m, 4H), 1.92-1.85 (m, 6H), 1.66-1.55 (M. 2H). 1.47 (t, J=5.6 Hz, 4H), 0.95 (s, 6H): LC-MS (M+H)⁺; 562.5.

### Example 38: (S)-3-(3-(3-methyl-1H-pyrazol-1-yl)phenyl)-3-((R)-1-(3-(5,6,7,8-tetrahydro-1,8-naphthyridin-2-yl)propyl)piperidine-3-carboxamido)propanoic acid (238)

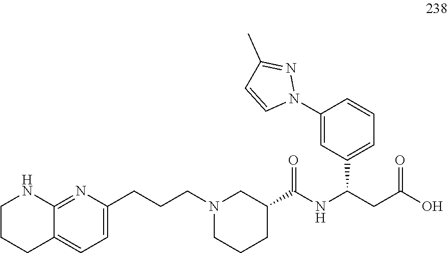 [SMILES: Cc1ccn(-c2cccc([C@H](CC(=O)O)NC(=O)[C@@H]3CCCN(CCCc4ccc5c(n4)NCCC5)C3)c2)n1]

Following the procedure illustrated in FIG. 3, Scheme 4, yielded compound 238. ¹H NMR (400 MHz, CDCl₃) δ 10.58 (s, 1H), 9.99 (br s, 1H), 7.75-7.72 (m, 2H), 7.43-7.41 (m, 1H), 7.30-7.29 (m, 2H), 7.14 (d, J=7.2 Hz, 1H), 6.17-6.15 (M. 2H), 5.58-5.56 (m, 1H), 3.36-3.34 (m, 2H), 2.97-2.86 (m, 5H), 2.67-2.61 (m, 3H), 2.50-2.48 (m, 2H), 2.43-2.39 (m, 1H), 2.33-2.30 (m, 4H), 1.83-1.79 (m, 7H), 1.61-1.58 (m, 2H); LC-MS (M+H)⁺: 531.0.

### Example 39: (S)-3-(3-(2-oxopyrrolidin-1-yl)phenyl)-3-((R)-1-(3-(5,6,7,8-tetrahydro-1,8-naphthyridin-2-yl)propyl)piperidine-3-carboxamido)propanoic acid (239)

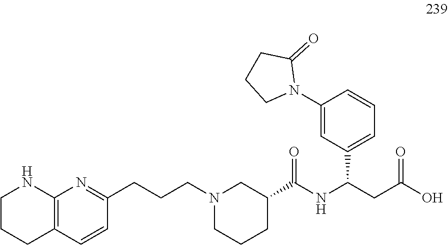 [SMILES: O=C(O)C[C@H](NC(=O)[C@@H]1CCCN(CCCc2ccc3c(n2)NCCC3)C1)c1cccc(N2CCCC2=O)c1]

Following the procedure illustrated in FIG. 3, Scheme 4, yielded compound 239. ¹H NMR (400 MHz, DMSO-d₆) δ 8.65 (d, J=8.4 Hz, 1H), 7.58 (s, 1H), 7.51-7.48 (m, 1H), 7.27 (t, J=8.0 Hz. 1H), 7.05 (d, J=7.2 Hz, 2H), 6.69 (s, 1H), 6.26 (d, J=7.6 Hz, 1H), 5.17-5.11 (m, 11H), 3.79 (t, J=6.8 Hz, 2H), 3.38-3.37 (m, 1H), 3.23 (t, J=5.4 Hz. 2H), 2.71-2.65 (m, 2H), 2.61-2.58 (m, 4H), 2.49-2.41 (m, 4H), 2.29-2.21 (m, 2H), 2.15-2.00 (M, 4H), 1.76-1.67 (m, 4H), 1.64-1.62 (m, 2H), 1.47-1.31 (m, 2H): LC-MS (M+H)⁺. 534.1.

### Example 40: (S)-3-([1,1′-biphenyl]-3-yl)-3-((R)-1-(3-(5,6,7,8-tetrahydro-1,8-naphthyridin-2-yl)propyl) piperidine-3-carboxamido)propanoic acid hydrochloride (240)

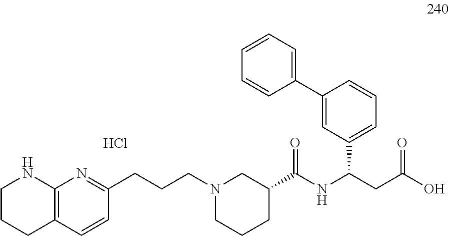 [SMILES: O=C(O)C[C@H](NC(=O)[C@@H]1CCCN(CCCc2ccc3c(n2)NCCC3)C1)c1cccc(-c2ccccc2)c1 ; Cl]

Following the procedure illustrated in FIG. 3, Scheme 4, yielded 240. ¹H NMR (400 MHz, DMSO-d₆) δ 14.00 (s, 1H), 10.72 (s, 1H), 8.80 (d, J=8.0 Hz, 1H), 7.95 (s, 1H), 7.64-7.60 (m, 4H), 7.54-7.46 (m, 3H), 7.43-7.37 (m, 2H), 7.29 (d, J=7.2 Hz, 1H), 6.68 (d, J=8.0 Hz, 1H), 5.25-5.22 (m, 1H), 3.06 (br s, 3H), 2.93 (d, J=8.8 Hz, 2H), 2.83-2.67 (m, 10H), 2.14-2.12 (m. 2H), 1.96-1.82 (m, 6H); LC-MS (M+H)⁺: 527.3.

### Example 41: (3S)-3-(3-(2-oxa-5-azabicyclo[2.2.2]octan-5-yl)phenyl)-3-((R)-1-(3-(5,6,7,8-tetrahydro-1,8-naphthyridin-2-yl)propyl)piperidine-3-carboxamido)propanoic acid (241)

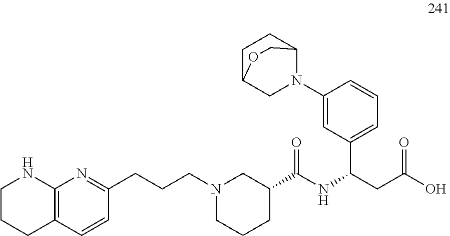 [SMILES: O=C(O)C[C@H](NC(=O)[C@@H]1CCCN(CCCc2ccc3c(n2)NCCC3)C1)c1cccc(N2CC3CCC2CO3)c1]

Following the procedure illustrated in FIG. 3, Scheme 4, yielded compound 241 whose isomers were separated using Prep-SFC to yield compound 241-A and compound 241-B.

Compound 241-A: ¹H NMR (400 MHz, DMSO-d₆) δ 8.56 (d, J=8.0 Hz, 1H), 7.10-7.05 (m, 2H), 6.58-6.50 (m, 4H), 6.28 (d, J=7.2 Hz, 1H), 5.14-5.08 (m, 1H), 3.97-3.90 (m, 5H), 3.64-3.62 (m, 2H), 3.28-3.22 (m, 3H), 2.99 (br s, 2H), 2.63-2.61 (m, 6H), 2.46-2.44 (m, 3H), 2.03-1.97 (m, 1H), 1.91-1.85 (m, 4H), 1.75-1.66 (m, 6H), 1.42-1.40 (m, 1H); LC-MS (M+H)⁺: 562.5.

Compound 241-B: ¹H NMR (400 MHz, DMSO-d₆) δ 8.52 (d, J=8.0 Hz, 1H), 7.10-7.05 (m, 2H), 6.57-6.50 (m, 4H), 6.28 (d, J=7.2 Hz, 1H), 5.13-5.08 (m, 1H), 3.96-3.90 (M. 5H), 3.63-3.61 (m, 2H), 3.28-3.22 (m, 4H), 2.92 (brs, 1H), 2.62-2.59 (m, 5H), 2.47-2.43 (m, 4H), 2.01-1.97 (m, 1H), 1.91-1.66 (m, 11H), 1.41-1.38 (m, 1H); LC-MS (M+H)⁺: 562.5.

### Example 42: (S)-3-((R)-1-(3-(5,6,7,8-tetrahydro-1,8-naphthyridin-2-yl)propyl)piperidine-3-carboxamido)-3-(3-(2,2,6,6-tetramethylmorpholino)phenyl)propanoic acid (242)

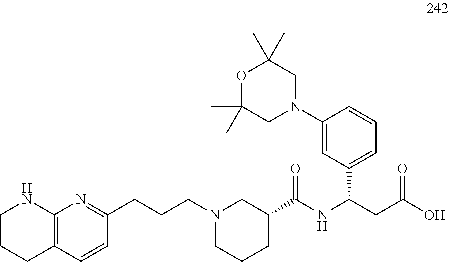 [SMILES: CC1(C)CN(c2cccc([C@H](CC(=O)O)NC(=O)[C@@H]3CCCN(CCCc4ccc5c(n4)NCCC5)C3)c2)CC(C)(C)O1]

Following the procedure illustrated in FIG. 3, Scheme 4, yielded compound 242. ¹H NMR (400 MHz, DMSO-d₆) δ 8.50 (d, J=8.0 Hz, 1H), 7.13 (t, J=8.0 Hz, 1H), 7.05 (d, J=7.2 Hz, 1H), 6.83 (s, 1H), 6.75-6.69 (m, 3H), 6.26 (d, J=7.2 Hz, 1H), 5.15-5.09 (m, 1H), 3.23 (t, J=4.8 Hz, 2H), 2.95 (s, 4H), 2.73-2.71 (M, 1H), 2.62-2.58 (m, 5H), 2.44 (t, J=7.2 Hz, 2H), 2.39-2.34 (m, 1H), 2.27-2.26 (m, 2H), 2.12-1.96 (M, 2H), 1.75-1.71 (m, 4H), 1.65-1.62 (m, 2H), 1.46-1.34 (m, 2H), 1.20 (s, 12H); LC-MS (M+H)⁺: 592.2.

### Example 43: (S)-3-(3-((2R,6R)-2,6-dimethylmorpholino)phenyl)-3-((R)-1-(3-(5,6,7,8-tetrahydro-1,8-naphthyridin-2-yl)propyl)piperidine-3-carboxamido)propanoic acid (243)

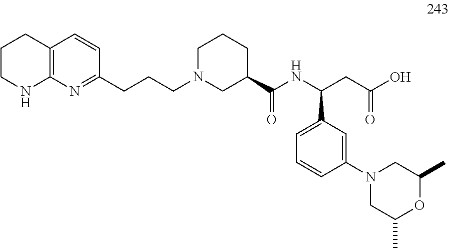 [SMILES: C[C@@H]1CN(c2cccc([C@H](CC(=O)O)NC(=O)[C@@H]3CCCN(CCCc4ccc5c(n4)NCCC5)C3)c2)C[C@@H](C)O1]

Following the procedure illustrated in FIG. 3, Scheme 4, yielded compound 243. ¹H NMR (400 MHz, CDCl₃) δ 10.72 (br s, 1H), 9.81 (br s, 1H), 7.16-7.14 (m, 2H), 6.96-6.92 (m, 2H), 6.67 (d, J=8.4 Hz, 1H), 6.20 (dd, J₁=7.2 Hz, J₂=1.2 Hz, 1H), 5.49-5.46 (m, 1H), 4.12-4.08 (m, 2H), 3.41-3.36 (M, 2H), 3.16-3.12 (m, 2H), 2.87-2.79 (m, 7H), 2.68-2.31 (m, 8H), 1.92-1.84 (m, 6H), 1.59 (br s, 2H), 1.27 (dd, J=6.4 Hz, J₂═0.8 Hz, 6H): LC-MS (M+H)⁺: 564.1.

### Example 44: 3-(3-cyclopropyl-4-fluorophenyl)-3-((R)-1-(3-(5,6,7,8-tetrahydro-1,8-naphthyridin-2-yl)propyl)piperidine-3-carboxamido)propanoic acid (244)

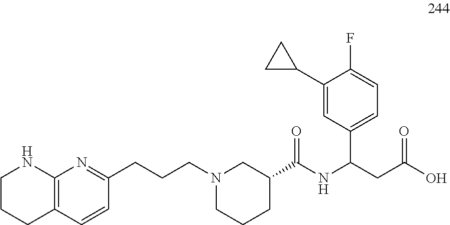 [SMILES: O=C(O)CC(NC(=O)[C@@H]1CCCN(CCCc2ccc3c(n2)NCCC3)C1)c1ccc(F)c(C2CC2)c1]

Following the procedure illustrated in FIG. 3, Scheme 4, yielded compound 244 which was separated into isomers, compound 244-A and compound 244-B using Prep-SFC.

Compound 244-A: ¹H NMR (400 MHz, CDCl₃) δ 10.82 (s, 1H), 9.35 (d, J=9.2 Hz, 1H), 7.20-7.18 (m, 2H), 7.05 (dd, J₁=7.2 Hz, J₂=2.0 Hz, 1H), 6.91-6.87 (m, 1H), 6.24 (d, J=7.2 Hz, 1H), 5.44-5.41 (m, 1H), 3.39-3.34 (m, 3H), 3.08-3.00 (m, 2H), 2.84-2.83 (m, 1H), 2.80-2.79 (m, 1H), 2.67 (t, J=6.0 Hz, 2H), 2.61-2.60 (m, 1H), 2.44-2.40 (m, 3H), 2.23 (d, J=13.6 Hz, 1H), 2.04-2.02 (m, 4H), 1.86-1.84 (m, 2H), 1.76-1.61 (m, 2H), 1.57-1.44 (m, 2H), 0.91-0.87 (m, 2H), 0.69-0.66 (m, 2H): LC-MS (M+H)⁺: 509.1.

Compound 244-B: ¹H NMR (400 MHz, CDCl₃) δ 10.68 (s, 1H), 9.84 (s, 1H), 7.19 (d, J=7.2 Hz. 1H), 7.16-7.12 (m, 1H), 6.95 (dd, J₁=7.2 Hz, J₂=2.0 Hz, 1H), 6.88-6.84 (m, 1H), 6.22 (d, J=7.2 Hz, 1H), 5.43-5.41 (m, 1H), 3.40 (t, J=4.0 Hz, 2H), 2.94 (br s, 2H), 2.83-2.81 (m, 3H), 2.60-2.63 (m, 4H), 2.52-2.46 (m, 2H), 2.41-2.30 (m, 3H), 2.02-1.97 (m, 2H), 1.90-1.85 (m, 2H), 1.80-1.77 (m, 2H), 1.59-1.50 (m, 2H), 0.89-0.87 (m, 2H), 0.64-0.63 (m, 2H); LC-MS (M+H)⁺: 509.1.

### Example 45: 3-(5-cyclopropyl-2-fluorophenyl)-3-((R)-1-(3-(5,6,7,8-tetrahydro-1,8-naphthyridin-2-yl)propyl)piperidine-3-carboxamido)propanoic acid (245)

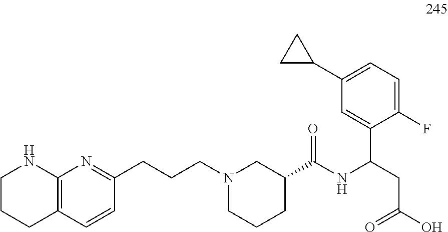 [SMILES: O=C(O)CC(NC(=O)[C@@H]1CCCN(CCCc2ccc3c(n2)NCCC3)C1)c1cc(C2CC2)ccc1F]

Following the procedure illustrated in FIG. 3, Scheme 4, yielded compound 245 which was separated into isomers compound 245-A and compound 245-B using Prep-SFC.

Compound 245-A: ¹H NMR (400 MHz, CDCl₃) δ 10.70 (s, 1H), 9.86 (s, 1H), 7.19 (d, J=7.6 Hz, 1H), 7.14 (d, J=7.6 Hz, 1H), 6.86-6.84 (m, 2H), 6.25 (d, J=7.2 Hz, 1H), 5.73-5.69 (m, 1H), 3.38-3.37 (m, 2H), 3.08-3.00 (m, 3H), 2.92-2.87 (m, 1H), 2.83-2.78 (m, 1H), 2.69-2.62 (m, 5H), 2.51-2.45 (m, 2H), 2.39-2.34 (m, 1H), 2.02-1.99 (m, 1H), 1.88-1.77 (m, 6H), 1.62-1.58 (m, 2H), 0.82-0.79 (m, 2H), 0.53-0.52 (m, 2H); LC-MS (M+H)⁺: 509.1.

Compound 245-B: ¹H NMR (400 MHz, CDCl₃) δ 10.61 (s, 1H), 7.20 (d, J=7.2 Hz, 1H), 7.14 (d, J=6.8 Hz, 11H), 6.89-6.84 (m, 2H), 6.26 (d, J=7.2 Hz, 1H), 5.66 (s, 1H), 3.41-3.38 (m, 2H), 2.95-2.67 (m, 14H), 1.93-1.79 (m, 8H), 0.84-0.81 (m, 2H), 0.55-0.53 (m, 2H); LC-MS (M+H)⁺: 509.1.

The following compounds set forth in Table 3, were prepared according to the general procedure illustrated in FIG. 3, Scheme 4 or analogous procedures thereto.

TABLE 3

| Compound | |
|---|---|
| No. | ¹H NMR Data |
| 344 | (400 MHz, DMSO-d₆) δ 8.63 (d, J = 7.2 Hz, 1H), 7.15-6.98 (m, 3H), |
| | 6.86-6.70 (m, 2H), 6.27 (d, J = 7.2 Hz, 1H), 5.41 (q, J = 7.2 Hz, 1H), |
| | 3.23 (t, J = 5.2 Hz, 2H), 2.73-2.67 (m, 1H), 2.63-2.58 (m, 4H), 2.46- |
| | 2.42 (m, 2H), 2.38-2.32 (m, 1H), 2.31-2.24 (m, 2H), 2.14-2.06 (m, |
| | 1H), 2.05-1.98 (m, 2H), 1.78-1.68 (m, 4H), 1.66-1.56 (m, 2H), 1.46- |
| | 1.31 (m, 2H), 1.25-1.22 (m, 1H), 1.00-0.90 (m, 2H), 0.73-0.64 (m, |
| | 2H). |
| 347 | (400 MHz, DMSO-d₆) δ 8.58 (d, J = 7.6 Hz, 1H), 7.10 (d, J = 7.2 Hz, |
| | 1H), 6.90-6.88 (m, 2H), 6.75-6.73 (m, 1H), 6.29 (d, J = 7.6 Hz, 1H), |
| | 5.11 (q, J = 8.0 Hz, 1H), 3.24-3.23 (m, 2H), 2.67-2.59 (m, 5H), 2.45- |
| | 2.12 (m, 8H), 1.92-1.85 (m, 1H), 1.79-1.67 (m, 4H), 1.58-1.42 (m, |
| | 4H), 0.94 (dd, J₁ = 8.0 Hz, J₂ = 4.8 Hz, 2H), 0.68-0.64 (m, 2H). |
| 533 | (400 MHz, CDCl₃) δ 10.68 (br s, 1H), 9.80 (br s, 1H), 7.29 (s, 1H), 7.26 |
| | (br s, 1H), 7.22-7.17 (m, 2H), 7.03 (d, J = 7.6 Hz, 1H), 6.21 (d, J = 7.2 |
| | Hz, 1H), 5.49-5.47 (m, 1H), 4.08-3.97 (m, 2H), 3.90-3.83 (m, 1H), |
| | 3.62 (t, J = 8.0 Hz, 1H), 3.44-3.29 (m, 3H), 2.99-2.76 (m, 5H), 2.73- |
| | 2.54 (m, 4H), 2.53-2.39 (m, 2H), 2.38-2.21 (m, 2H), 2.10-1.91 (m, |
| | 4H), 1.89-1.74 (m, 4H), 1.69-1.54 (m, 2H). |
| 534 | (400 MHz, CDCl₃) δ 10.73-10.66 (m, 1H), 9.87-9.78 (m, 1H), 7.30 (s, |
| | 1H), 7.26 (br s, 1H), 7.22-7.17 (m, 2H), 7.02 (d, J = 7.6 Hz, 1H), 6.21 |
| | (d, J = 6.8 Hz, 1H), 5.50-5.47 (m, 1H), 4.14-3.96 (m, 2H), 3.88 (q, J = |
| | 8.0 Hz, 1H), 3.62 (t, J = 8.0 Hz, 1H), 3.46-3.30 (m, 3H), 3.03-2.79 (m, |
| | 5H), 2.67 (t, J = 6.0 Hz, 3H), 2.57-2.22 (m, 5H), 2.11-1.89 (m, 4H), |
| | 1.89-1.74 (m, 4H), 1.68-1.54 (m, 2H). |
| 536 | (400 MHz, DMSO-d₆) δ 8.60 (d, J = 8.0 Hz, 1H), 7.49 (s, 1H), 7.42- |
| | 7.35 (m, 2H), 7.31 (t, J = 2.2 Hz, 1H), 7.17 (d, J = 7.2 Hz, 1H), 7.05 (d, |
| | J = 7.2 Hz, 1H), 6.75 (s, 1H), 6.27-6.25 (m, 3H), 5.25-5.19 (m, 1H), |
| | 3.22 (s, 2H), 2.75-2.72 (m, 1H), 2.69-2.66 (m, 2H), 2.60 (t, J = 6.0 Hz, |
| | 2H), 2.42-2.41 (m, 2H), 2.34-2.32 (m, 1H), 2.30-2.26 (m, 3H), 2.12- |
| | 2.08 (m, 1H), 2.01-1.95 (m, 1H), 1.75-1.69 (m, 4H), 1.63 (d, J = 9.2 |
| | Hz, 2H), 1.44-1.36 (m, 2H). |
| 537 | (400 MHz, DMSO-d₆) δ 8.67 (br d, J = 7.4 Hz, 1H), 8.44 (d, J = 2.4 Hz, |
| | 1H), 7.81 (s, 1H), 7.73 (d, J = 1.6 Hz, 1H), 7.66 (dd, J₁ = 8.0 Hz, J₂ = 1.4 |
| | Hz, 1H), 7.41 (t, J = 8.0 Hz, 1H), 7.23 (d, J = 7.8 Hz, 1H), 7.05 (d, J = |
| | 7.2 Hz, 1H), 6.80 (br s, 1H), 6.53 (t, J = 2.0 Hz, 1H), 6.26 (d, J = 7.2 Hz, |
| | 1H), 5.23 (q, J = 6.8 Hz, 1H), 3.22 (br t, J = 5.4 Hz, 2H), 2.75 (br d, J = |
| | 10.0 Hz, 1H), 2.69 (br d, J = 7.2 Hz, 2H), 2.59 (br t, J = 6.0 Hz, 3H), |
| | 2.47-2.38 (m, 3H), 2.34-2.26 (m, 2H), 2.15 (br s, 1H), 2.02 (br s, 1H), |
| | 1.77-1.68 (m, 4H), 1.63 (br d, J = 9.6 Hz, 2H), 1.49-1.30 (m, 2H). |
| 558 | (400 MHz, DMSO-d₆) δ 8.56 (d, J = 7.2 Hz, 1H), 7.06 (d, J = 7.2 Hz, |
| | 1H), 6.87-6.83 (m, 2H), 6.77-6.70 (m, 2H), 6.27 (d, J = 7.2 Hz, 1H), |
| | 5.11 (q, J = 8.0 Hz, 1H), 3.23 (t, J = 5.2 Hz, 2H), 2.73-2.67 (m, 1H), |
| | 2.62-2.59 (m, 4H), 2.45 (t, J = 7.2 Hz, 2H), 2.39-2.25 (m, 4H), 2.12- |
| | 1.97 (m, 2H), 1.93-1.86 (m, 1H), 1.77-1.68 (m, 4H), 1.64-1.62 (m, |
| | 2H), 1.45-1.33 (m, 2H), 0.97-0.92 (m, 2H), 0.68-0.66 (m, 2H). |
| 559 | (400 MHz, DMSO-d₆) δ 8.68 (d, J = 7.2 Hz, 1H), 7.21-7.01 (m, 3H), |
| | 6.85 (t, J = 7.6 Hz, 1H), 6.30 (d, J = 7.2 Hz, 1H), 5.44 (q, J = 7.2 Hz, |
| | 1H), 3.26-3.21 (m, 2H), 3.13-2.94 (m, 2H), 2.87-2.72 (m, 2H), 2.67- |
| | 2.58 (m, 5H), 2.10-1.94 (m, 2H), 1.88 (s, 2H), 1.80-1.67 (m, 4H), 1.66- |
| | 1.36 (m, 3H), 1.31-1.19 (m, 2H), 1.02-0.92 (m, 2H), 0.74-0.64 (m, 2H). |
| 582 | (400 MHz, CDCl₃) δ 10.68 (s, 1H), 9.79 (br s, 1H), 7.18-7.11 (m, 2H), |
| | 6.92-6.87 (m, 2H), 6.64 (dd, J₁ = 8.0 Hz, J₂ = 2.4 Hz, 1H), 6.19 (d, J = |
| | 6.8 Hz, 1H), 5.48-5.46 (m, 1H), 3.97-3.94 (m, 1H), 3.85-3.84 (m, |
| | 1H), 3.67-3.64 (m, 2H), 3.40-3.38 (m, 2H), 3.06-3.04 (m, 2H), 2.89- |
| | 2.84 (m, 5H), 2.68-2.65 (m, 4H), 2.42-2.30 (m, 3H), 1.89-1.72 (m, |
| | 7H), 1.60 (br s, 2H), 1.15 (d, J = 6.4 Hz, 3H), 0.86 (d, J = 6.4 Hz, 3H). |
| 583 | (400 MHz, CDCl₃) δ 10.69 (s, 1H), 9.78 (br s, 1H), 7.18-7.11 (m, 2H), |
| | 6.93 (s, 1H), 6.88 (d, J = 7.6 Hz, 1H), 6.62 (dd, J₁ = 8.0 Hz, J₂ = 2.0 Hz, |
| | 1H), 6.19 (d, J = 6.8 Hz, 1H), 5.47-5.45 (m, 1H), 3.95-3.92 (m, 1H), |
| | 3.84-3.82 (m, 1H), 3.66-3.62 (m, 2H), 3.40-3.39 (m, 2H), 3.05-3.03 |
| | (m, 2H), 2.87-2.83 (m, 5H), 2.67-2.65 (m, 4H), 2.41-2.29 (m, 3H), |
| | 1.89-1.72 (m, 7H), 1.59 (br s, 2H), 1.12 (d, J = 6.4 Hz, 3H), 0.85 (d, J = |
| | 6.8 Hz, 3H). |
| 584 | (400 MHz, CDCl₃) δ 10.73 (s, 1H), 9.86 (br s, 1H), 7.18-7.16 (m, 3H), |
| | 7.11-7.09 (m, 1H), 6.86-6.84 (m, 1H), 6.20 (d, J = 7.2 Hz, 1H), 5.50- |
| | 5.48 (m, 1H), 3.81-3.80 (m, 2H), 3.44-3.39 (m, 3H), 2.92-2.85 (m, |
| | 8H), 2.67-2.65 (m, 3H), 2.54-2.30 (m, 4H), 1.91-1.84 (m, 7H), 1.59 |
| | (br s, 2H), 1.23 (d, J = 6.0 Hz, 3H), 0.83 (d, J = 6.0 Hz, 3H). |
| 585 | (400 MHz, CDCl₃) δ 10.68 (s, 1H), 9.86 (br s, 1H), 7.18-7.10 (m, 4H), |
| | 6.84 (d, J = 8.0 Hz, 1H), 6.20 (d, J = 7.2 Hz, 1H), 5.51-5.49 (m, 1H), |
| | 3.82-3.75 (m, 2H), 3.45-3.39 (m, 3H), 2.96-2.86 (m, 8H), 2.66-2.65 |
| | (m, 3H), 2.54-2.29 (m, 4H), 1.91-1.84 (m, 7H), 1.59 (br s, 2H), 1.26 |
| | (d, J = 6.0 Hz, 3H), 0.84 (d, J = 6.0 Hz, 3H). |
| 586 | (400 MHz, CDCl₃) δ 10.7 (s, 1H), 9.94 (s, 1H), 7.34-7.29 (m, 3H), 7.19 |
| | (d, J = 7.2 Hz, 1H), 7.05 (d, J = 6.4 Hz, 1H), 6.43 (s, 1H), 6.20 (d, J = |
| | 7.2 Hz, 1H), 5.83 (s, 1H), 5.54 (s, 1H), 3.40 (s, 2H), 2.98-2.79 (m, 6H), |
| | 2.67 (t, J = 6.00 Hz, 4H), 2.33 (s, 4H), 2.11 (s, 3H), 2.07 (s, 3H), 1.94- |
| | 1.83 (m, 6H), 1.73 (s, 1H), 1.61 (s, 1H). |
| 587 | (400 MHz, DMSO-d₆) δ 8.50 (d, J = 8.4 Hz, 1H), 7.11-7.04 (m, 2H), |
| | 6.83 (s, 1H), 6.73 (d, J = 8.4 Hz, 1H), 6.65 (d, J = 7.2 Hz, 2H), 6.26 (d, |
| | J = 7.2 Hz, 1H), 5.11 (q, J = 7.6 Hz, 1H), 3.23 (s, 2H), 2.78 (s, 4H), 2.74- |
| | 2.71 (m, 1H), 2.61-2.60 (m, 4H), 2.46-2.42 (m, 3H), 2.34-2.28 (m, |
| | 3H), 2.11-1.99(m, 2H), 1.76-1.63 (m, 6H), 1.45-1.34 (m, 2H), 1.22 |
| | (s, 2H), 0.98-0.97 (m, 12H). |

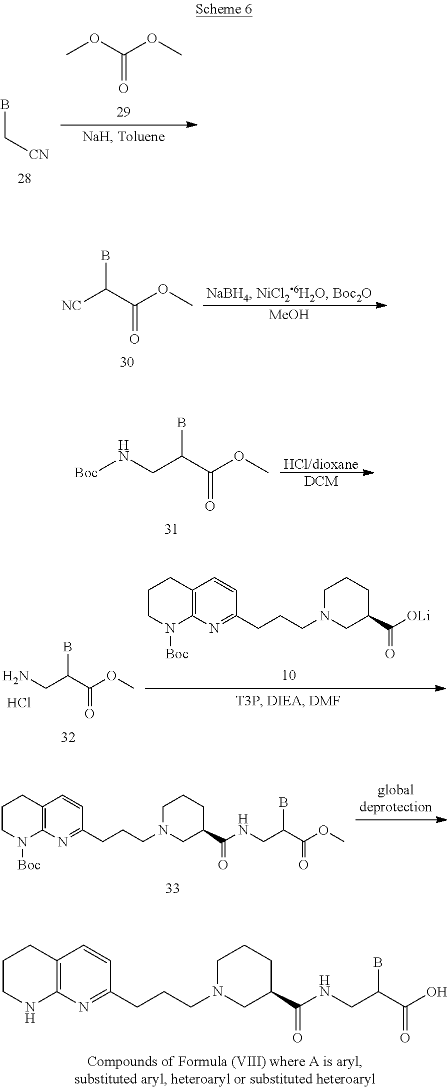

### FIG. 4

FIG. 4, Scheme 6, illustrates a general synthesis of compounds of Formula (VIII).

### General Procedure for Preparation of Compound 30

To a solution of nitrile 28 (1.00 eq) in toluene was added NaH (1.50 eq) at 0° C., the mixture warmed to 20° C., stirred for 0.5 hrs and dimethyl carbonate 29 (1.20 eq) was added. The resulting mixture was stirred at 80° C. for 2 hrs, quenched with saturated NH₄Cl solution at 0° C., and extracted with EtOAc. The combined organic extracts were dried over Na₂SO₄ and concentrated under vacuum to give crude compound 30 which was purified by column chromatography or used directly for next step.

### General Procedure for Preparation of Compound 31

To a stirred solution of compound 30 (1.00 eq) in MeOH was added Boc₂O (2.00 eq), NiCl₂·6H₂O (0.100 eq) and NaBH₄ (7.00 eq) at 0° C. The mixture was warmed to 20° C., stirred for 6 hrs, quenched by addition of 40.0 mL of MeOH, stirred for 0.5 hr, and filtered through a celite pad. The filtrate was concentrated to give a residue, which was purified by column chromatography to give compound 31, whose identity confirmed by ¹H NMR and LC-MS. The isomers of compound 31 were separated (if needed) by Prep-SFC (column: Phenomenex-Cellulose-2 (250 mm*30 mm, 10 um), mobile phase: [0.1% NH₃H₂O IPA]).

### General Procedure for Preparation of Compound 32

To a solution of compound 31 (racemate or pure stereoisomer) (1.00 eq) in DCM was added HCl/dioxane (5.08 eq) at 0° C., which was then stirred at 25° C. for 2 hrs. The reaction mixture was concentrated obtain crude compound 32 which was purified by column chromatography or used directly for next step.

### General Procedure for Preparation of Compound 33

To a solution of compound 32 (1.20 eq) and compound 10 (1.00 eq) in DMF was added DIEA (3.00 eq) and T3P (1.50 eq), and the mixture was stirred at 25° C. for 3 hrs and concentrated to provide a residue. The residue was purified by prep-HPLC (column: Waters Xbridge 150*25 mm*5 um: mobile phase: [water (10 mM NH₄HCO₃)−ACN]) to provide compound 33.

### General Procedure for Preparation of Compounds of Formula (VIII)

To a solution of compound 33 (1.00 eq) in H₂O was added HCl/dioxane (90.4 eq). The mixture was stirred at 60° C. for 4 hrs, concentrated and purified by prep-HPLC (column: 3_Phenomenex Luna C18 75*30 mm*3 um: mobile phase. [water (0.05% HCl)−ACN]) to provide a compound of formula (VIII).

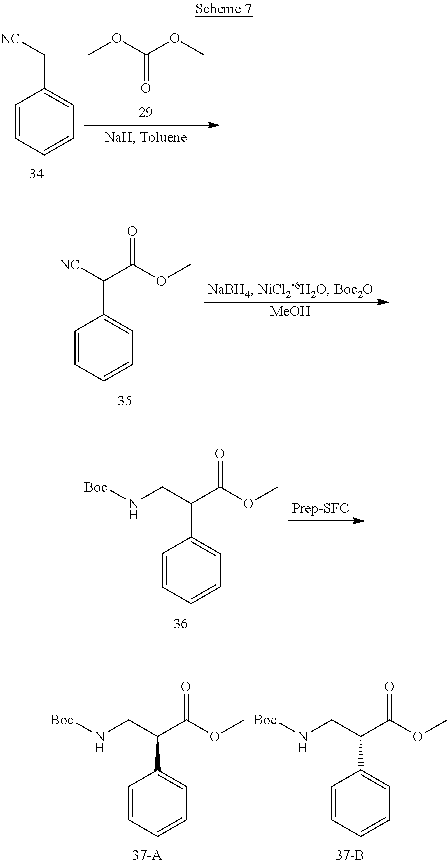

Scheme 7, above, illustrates preparation of intermediates 37-A and 37-B for the synthesis of compounds 246 and 247, which exemplify the preparation of compounds of Formula (VIII) as described in FIG. 4, Scheme 6.

### Preparation of Compound 35

To a solution of nitrile 34 (1.00 eq) in toluene was added NaH (2.56 g, 64.0 mmol, 60.0% purity, 1.50 eq) at 0° C., the mixture was warmed to 20° C., stirred for 0.5 h and compound 29 (4.61 g, 51.2 mmol, 4.31 mL, 1.20 eq) was added. The resulting mixture was stirred at 80° C. for 2 hrs. TLC (Petroleum ether: Ethyl acetate=5:1, R_{f}=0.24) indicated compound 34 was consumed completely, and one new spot was formed. The reaction mixture was quenched by saturated NH₄Cl solution (150 mL) at 0° C., extracted with EtOAc (80.0 mL*3). The combined organic layers were dried over Na₂SO₄, concentrated under vacuum to give crude compound 35 (7.00 g) was obtained as orange oil. ¹H NMR (400 MHz, CDCl₃) δ 7.48-7.42 (m, 5H), 4.75 (s, 1H), 3.82 (s, 3H).

### Preparation of Compound 36

To a stirred solution of compound 35 (800 mg, 4.57 mmol, 1.00 eq) in MeOH (40.0 mL) was added Boc₂O (1.99 g, 9.13 mmol, 2.10 mL. 2.00 eq), NiCl₂·6H₂O (109 mg, 457 umol, 0.100 eq), NaBH₄ (1.21 g, 32.0 mmol, 7.00 eq) at 0° C. The mixture was warmed to 20° C., and stirred for 6 hrs. TLC (Petroleum ether: Ethyl acetate=4:1, R_{f}═0.44) indicated compound 35 was consumed completely, and several new spots were formed. The reaction mixture was quenched by adding 40.0 mL of MeOH, stirred for 0.5 hrs and filtered through a celite pad. The filtrate was concentrated under vacuum to give a residue. The residue was purified by column chromatography (SiO₂, Petroleum ether: Ethyl acetate=60:1 to 20:1) to provide compound 36 (480 mg, 1.72 mmol, 37.6% yield) as a colorless oil. ¹H NMR (400 MHz, CDCl₃) δ 7.36-7.25 (m, 5H), 4.89 (br s, 1H), 3.90-3.88 (m, 1H), 3.70 (s, 3H), 3.60-3.53 (m, 2H), 1.43 (s, 9H); LC-MS (M-99)⁺: 180.3.

### Preparation of Compounds 37-A and 37-B

The isomers of compound 36 were separated by Prep-SFC (column: Phenomenex-Cellulose-2 (250 mm*30 mm, 10 um); mobile phase: [0.1% NH₃H₂O IPA]; B %: 15%-15%, 5.0 min; 280 min). Compound 37-A (220 mg, 787.60 umol, 36.67% yield) was obtained as colorless oil: LC-MS (M-99)⁺: 180.1; SFC (RT=0.730). Compound 37-B (250 mg, 895.00 umol, 41.67% yield) was obtained as colorless oil: LC-MS (M-99)⁺: 180.1; SFC (RT=0.848).

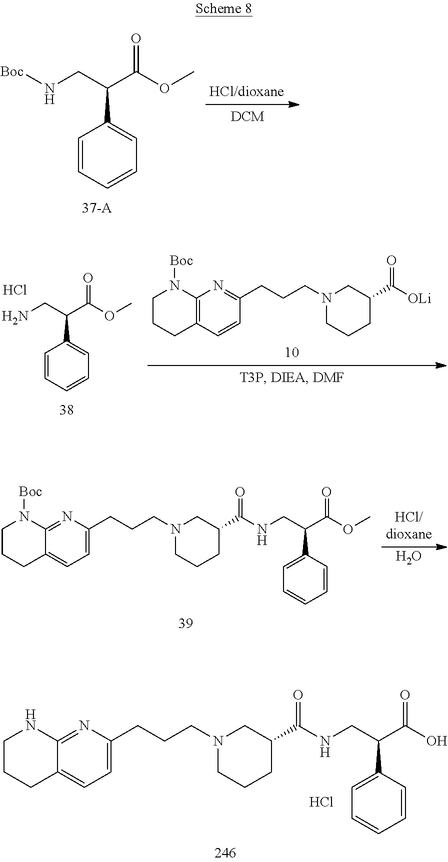

Scheme 8, above, illustrates preparation of compound 246, which exemplifies the pc preparation of compounds of Formula (VIII) as described in FIG. 4, Scheme 6.

### Preparation of Compound 38

To a solution of compound 37-A (220 mg, 788 umol, 1.00 eq) in DCM (10.0 mL) was added HCl/dioxane (4.00 M, 1.00 mL, 5.08 eq) at 0° C., and the mixture stirred at 25° C. for 2 hrs. TLC (Petroleum ether: Ethyl acetate=3:1. R_{f}=0.01) indicated compound 37-A was consumed completely, and one new spot with larger polarity was formed. The reaction mixture was concentrated under vacuum to give a residue. Compound 38 (170 mg, crude, HCl) was obtained as white solid. LC-MS (M-99)⁺: 180.2.

### Preparation of Compound 39

To a solution of compound 38 (44.1 mg, 205 umol, 1.20 eq, HCl) and compound 10 (70.0 mg, 171 umol, 1.00 eq, Li) in DMF (2.00 mL) was added DIEA (66.1 mg, 512 umol, 89.1 uL, 3.00 eq) and T3P (163 mg, 256 umol, 152 uL, 50.0% purity, 1.50 eq) and the mixture was stirred at 25° C. for 3 hrs. LC-MS indicated detection of one major peak with desired mass. The reaction mixture was purified by prep-HPLC (column: Waters Xbridge 150*25 mm*5 um; mobile phase: [water (10 mM NH₄HCO₃)−ACN]; B %: 51%-81%, 10 min). 45 mg of compound 39 was obtained as colorless gum. LC-MS (M+H)⁺: 565.6; ¹H NMR (400 MHz, CDCl₃) δ 8.39 (br s, 1H), 7.34-7.29 (m, 5H), 7.26-7.24 (m, 1H), 6.85 (d, J=7.6 Hz, 1H). 3.97 (t, J=7.6 Hz, 1H), 3.78-3.69 (m, 4H), 3.67 (s, 3H), 2.76-2.61 (m, 6H), 2.05-2.01 (m, 1H), 2.36-2.22 (m, 3H), 2.05-2.01 (m, 1H), 1.96-1.89 (m, 2H), 1.85-1.77 (m, 3H), 1.52-1.46 (m, 12H). Example 46: (S)-2-phenyl-3-((R)-1-(3-(5,6,7,8-tetrahydro-1,8-naphthyridin-2-yl)propyl)piperidine-3-carboxamido)propanoic acid (246)

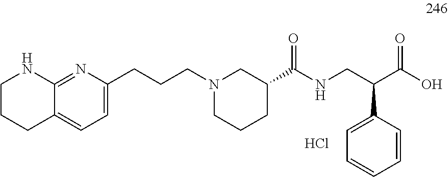 [SMILES: O=C(NC[C@@H](C(=O)O)c1ccccc1)[C@@H]1CCCN(CCCc2ccc3c(n2)NCCC3)C1 ; Cl]

To a solution of compound 39 (40.0 mg, 70.8 umol, 1.00 eq) in H₂O (0.200 mL) was added HCl/dioxane (4.00 M, 1.60 mL, 90.4 eq). The mixture was stirred at 60° C. for 4 hrs. LC-MS indicated compound 39 was completely consumed and detected a major peak with the desired mass.

The reaction mixture was purified by prep-HPLC (column: 3_Phenomenex Luna C18 75*30 mm*3 um: mobile phase: [water (0.05% HCl)−ACN]; B %: 5%-25%, 7 min). 22.28 mg of 246 (96.3% purity, HCl) was obtained as colorless oil. LC-MS (M+H)⁺: 451.4; ¹H NMR (400 MHz, DMSO-d₆) δ 14.38 (br s, 1H), 10.95 (br s. 1H), 8.35 (t, J=5.6 Hz, 1H), 8.09 (s, 1H), 7.63 (d, J=7.2 Hz, 1H), 7.36-7.23 (m, 5H), 6.67 (d, J=7.6 Hz, 1H), 3.76 (t, J=7.2 Hz, 1H), 3.60-3.28 (m, 7H), 3.03 (br s, 2H), 2.91-2.66 (m, 7H), 2.16-2.07 (m, 2H), 1.92-1.80 (m, 4H), 1.40-1.30 (m, 1H); HPLC purity: 96.3% (254 nm); SFC chiral purity: 100%.

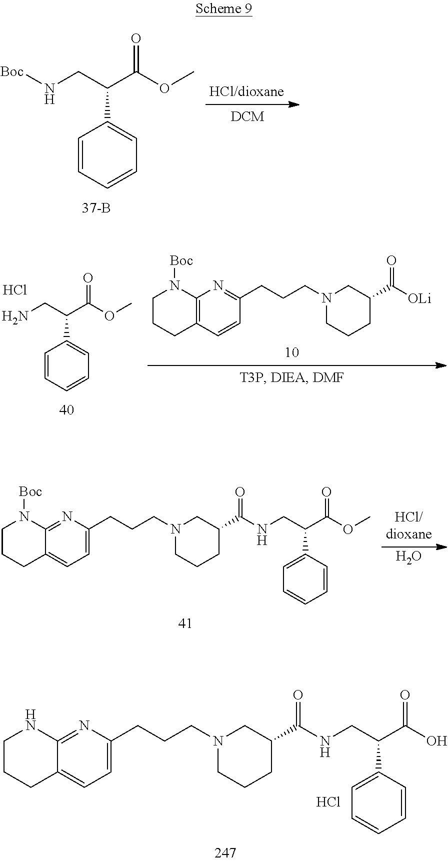

Scheme 9, above, illustrates preparation of compound 247, which exemplifies the preparation of compounds of Formula (VIII) as described in FIG. 4, Scheme 6.

### Preparation of Compound 40

To a solution of compound 37-B (250 mg, 895 umol, 1.00 eq) in DCM (10.0 mL) was added HCl/dioxane (4.00 M, 1.00 mL, 4.47 eq) at 0° C. with stirring at 25° C. for 2 hrs. TLC (Petroleum ether: Ethyl acetate=3:1, R_{f}=0.01) indicated compound 37-B was consumed completely, and one new spot with larger polarity was formed. The reaction mixture was concentrated under vacuum to give a residue. Crude compound 40 (180 mg, HCl) was obtained as white solid. LC-MS (M+H)⁺: 180.1.

### Preparation of Compound 41

To a solution of compound 40 (36.7 mg, 205 umol, 1.20 eq, HCl) and compound 10 (70.0 mg, 171 umol, 1.00 eq, Li) in DMF (2.00 mL) was added DIEA (66.1 mg, 512 umol, 89.1 uL, 3.00 eq) and T3P (163 mg, 256 umol, 152 uL, 50.0% purity, 1.50 eq), and the mixture was stirred at 25° C. for 3 hrs. LC-MS indicated detection of one major peak with the desired mass. The reaction mixture was purified by prep-HPLC (column: Waters Xbridge 150*25 mm*5 um: mobile phase: [water (10 mM NH₄HCOs)−ACN]: B %: 51%-81%, 10 min). 45 mg of compound 41 was obtained as light-yellow gum. LC-MS (M+H)⁺: 565.6: ¹H NMR (400 MHz, CDCl₃) 8.24 (br s, 1H), 7.33-7.26 (M, 5H), 7.25-7.23 (m, 1H), 6.83 (d, J=7.6 Hz, 1H), 3.95-3.91 (m, 1H), 3.88-3.80 (m, 1H), 3.77-3.74 (m, 2H), 3.68-3.60 (m, 4H), 2.74 (t, J=6.8 Hz, 3H), 2.65 (t, J=7.6 Hz., 2H), 2.57-2.55 (m, 1H), 2.44-2.41 (m, 1H), 2.37-2.25 (m, 3H), 2.07 (br s. 1H), 1.96-1.89 (m, 2H), 1.88-1.81 (m, 3H), 1.52 (s, 9H), 1.49-1.44 (m, 3H); SFC chiral purity: 100%.

### Example 47: (R)-2-phenyl-3-((R)-1-(3-(5,6,7,8-tetrahydro-1,8-naphthyridin-2-yl)propyl)piperidine-3-carboxamido)propanoic acid (247)

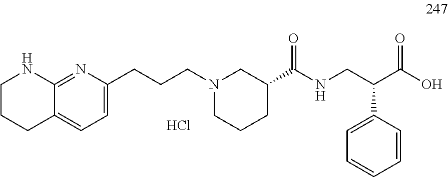 [SMILES: O=C(NC[C@H](C(=O)O)c1ccccc1)[C@@H]1CCCN(CCCc2ccc3c(n2)NCCC3)C1 ; Cl]

To a solution of compound 41 (40.0 mg, 70.8 umol, 1.00 eq) in H₂O (0.200 mL) was added HCl/dioxane (4.00 M, 1.60 mL, 904 eq). The mixture was stirred at 60° C. for 2 hrs. LC-MS indicated compound 41 was consumed completely, a major peak with desired mass was detected.

The reaction mixture was concentrated under vacuum to give a residue. The residue was purified by prep-HPLC (column: 3_Phenomenex Luna C18 75*30 mm*3 um; mobile phase; [water (0.05% HCl)−ACN]; B %: 5%-25%, 7 min). 33.77 mg of 247 (97.1% purity, HCl) was obtained as light-yellow oil. LC-MS (M+H)⁺: 451.3; ¹H NMR (400 MHz, DMSO-d₆) δ 14.42 (br s, 1H), 10.96 (br s. 1H), 8.34 (t, J=5.6 Hz. 1H). 8.11 (s, 1H). 7.63 (d, J=7.2 Hz, 1H), 7.36-7.26 (m, 5H), 6.68 (d, J=7.2 Hz, 1H), 3.75 (t, J=7.6 Hz, 1H), 3.53-3.32 (m, 6H), 3.06-2.98 (m, 2H), 2.91-2.73 (m, 7H), 2.18-2.10 (m, 2H), 1.89-1.71 (m, 5H), 1.33-1.26 (m, 1H); HPLC purity: 97.1% (254 nm); SFC chiral purity: 98.6%.

### Example 48: 3-((R)-1-(3-(5,6,7,8-tetrahydro-1,8-naphthyridin-2-yl)propyl)piperidine-3-carboxamido)-2-(5,6,7,8-tetrahydronaphthalen-2-yl)propanoic acid (248)

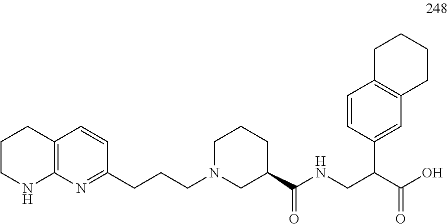 [SMILES: O=C(O)C(CNC(=O)[C@@H]1CCCN(CCCc2ccc3c(n2)NCCC3)C1)c1ccc2c(c1)CCCC2]

Following the procedure illustrated in FIG. 4, Scheme 6 and exemplified in Schemes 7, 8 and 9, yielded compound 248 as isomers 248-A and 248-B.

Compound 248-A: ¹H NMR (400 MHz, CDCl₃) δ 10.47 (s, 1H), 8.83 (d, J=5.2 Hz, 1H), 7.23 (d, J=7.2 Hz, 1H), 7.10-7.07 (m, 2H), 6.98 (d, J=8.0 Hz, 1H), 6.28 (d, J=7.2 Hz, 1H), 4.03-3.95 (m, 1H), 3.59 (dd, J₁=10.0 Hz, J₂=2.8 Hz. 1H), 3.54-3.44 (m, 1H), 3.40 (t, J=5.2 Hz. 2H), 3.14-3.03 (m, 2H), 2.93-2.85 (m, 1H), 2.84-2.76 (m, 1H), 2.76-2.66 (m, 6H), 2.56-2.50 (m, 1H), 2.36-2.21 (m, 2H), 2.16-2.04 (m, 2H), 2.03-1.94 (m, 1H), 1.91-1.83 (m, 2H), 1.81-1.65 (m, 7H), 1.56-1.45 (m, 2H); LC-MS (M+H)⁺: 505.3.

Compound 248-B: ¹H NMR (400 MHz, CDCl₃) S 10.80 (s, 1H), 8.70-8.69 (m, 1H), 7.23 (d, J=7.6 Hz, 1H), 7.14-7.10 (m, 2H), 6.99 (d, J=7.6 Hz, 1H), 6.29 (d, J=7.2 Hz, 1H), 3.94-3.86 (m, 1H), 3.70 (dd. J₁=10.8 Hz, J₂=3.2 Hz, 1H), 3.46-3.36 (m, 3H), 3.23-3.10 (m, 2H), 3.01 (br d, J=10.4 Hz, 1H), 2.82-2.47 (m, 8H), 2.35-2.18 (m, 2H), 2.09-1.95 (m, 2H), 1.92-1.83 (m, 4H), 1.82-1.56 (m, 6H), 1.55-1.39 (m, 2H): LC-MS (M+H)⁺: 505.3.

### Example 49: 2-(naphthalen-2-yl)-3-((R)-1-(3-(5,6,7,8-tetrahydro-1,8-naphthyridin-2-yl)propyl) piperidine-3-carboxamido)propanoic acid (249)

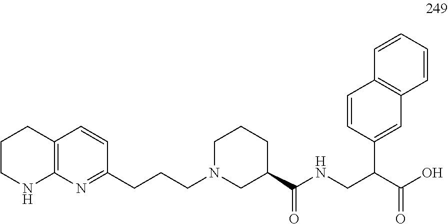 [SMILES: O=C(O)C(CNC(=O)[C@@H]1CCCN(CCCc2ccc3c(n2)NCCC3)C1)c1ccc2ccccc2c1]

Following the procedure illustrated in FIG. 4, Scheme 6 and exemplified in Schemes 7, 8 and 9, yielded compound 249 as isomers 249-A and 249-B.

Compound 249-A: ¹H NMR (400 MHz, CDCl₃) δ 10.52 (s, 1H), 8.91 (br s, 1H), 7.85 (s, 1H), 7.80-7.77 (m, 3H), 7.54 (dd. J₁=8.8 Hz, J₂=2.0 Hz, 1H), 7.44-7.37 (m, 2H), 7.25 (d, J=7.2 Hz, 1H), 6.31 (d, J=7.2 Hz, 1H), 4.09-4.03 (m, 1H), 3.85 (dd, J₁=9.6 Hz, J₂=2.8 Hz, 1H), 3.71-3.63 (m, 1H), 3.39 (t, J=5.2 Hz, 2H), 3.13-3.01 (m, 2H), 2.93-2.80 (m, 2H), 2.70 (t, J=6.0 Hz, 2H), 2.56-2.47 (m, 1H), 2.41-2.23 (m, 2H), 2.21-2.11 (m, 1H), 2.11-1.98 (m, 2H), 1.90-1.75 (m, 4H), 1.61-1.40 (m, 2H); LC-MS (M+H)⁺: 501.3.

Compound 249-B: ¹H NMR (400 MHz, CDCl₃) δ 10.85 (s, 1H), 8.80 (s, 1H), 7.88 (s, 1H), 7.83-7.76 (m, 3H), 7.58 (dd, J=8.8 Hz, J₂=2.0 Hz, 1H), 7.45-7.37 (m, 2H), 7.25 (d, J=7.2 Hz, 1H), 6.31 (d, J=7.2 Hz, 1H), 4.05-3.91 (m, 2H), 3.64-3.51 (m, 1H), 3.39 (t, J=5.2 Hz, 2H), 3.26-3.14 (m, 2H), 3.01 (d, J=10.0 Hz, 1H), 2.70 (t, J=6.4 Hz, 2H), 2.67-2.54 (m, 2H), 2.37-2.25 (m, 2H), 2.07-1.97 (m, 2H), 1.95-1.76 (m, 5H), 1.74-1.59 (m, 1H), 1.53-1.40 (m, 2H); LC-MS (M+H)⁺: 501.3.

### Example 50: 2-(naphthalen-1-yl)-3-((R)-1-(3-(5,6,7,8-tetrahydro-1,8-naphthyridin-2-yl)propyl) piperidine-3-carboxamido)propanoic acid (250)

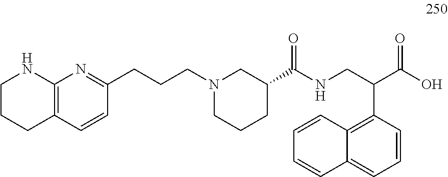 [SMILES: O=C(O)C(CNC(=O)[C@@H]1CCCN(CCCc2ccc3c(n2)NCCC3)C1)c1cccc2ccccc12]

Following the procedure illustrated in FIG. 4, Scheme 6 and exemplified in Schemes 7, 8 and 9, yielded compound 250 as isomers 250-A and 250-B.

250-A: ¹H NMR (400 MHz, CDCl₃) δ 10.93 (s, 1H), 8.91-8.82 (m, 1H), 8.57 (d, J=8.4 Hz, 1H), 7.82 (d, J=8.0 Hz, 1H), 7.73 (d, J=8.4 Hz, 1H), 7.63-7.51 (m, 2H), 7.48-7.38 (m, 2H), 7.26 (d, J=7.2 Hz, 1H), 6.33 (d, J=7.2 Hz. 1H), 4.63 (dd, J₁=10.8 Hz, J₂=3.2 Hz, 1H), 4.08-4.01 (m, 1H), 3.50-3.44 (m, 1H), 3.39 (t, J=5.2 Hz, 2H), 3.32-3.21 (m, 2H), 3.03 (d, J=10.0 Hz, 1H), 2.70 (t, J=6.4 Hz, 2H), 2.63-2.60 (m, 1H), 2.31 (t, J=4.8 Hz, 2H), 2.07-2.00 (m. 3H), 1.92-1.80 (m, 5H), 1.79-1.68 (m, 1H), 1.55-1.47 (m, 2H); LC-MS (M+H)⁺: 501.5.

Compound 250-B: ¹H NMR (400 MHz, CDCl₃) δ 10.37 (s. 1H), 8.78 (br s, 1H), 8.35 (d, J=8.4 Hz, 1H), 7.83 (d, J=8.4 Hz, 1H), 7.73 (d, J=8.0 Hz, 1H), 7.60-7.50 (m, 2H), 7.50-7.36 (m, 2H), 7.25 (d, J=7.6 Hz, 1H), 6.31 (d, J=7.2 Hz, 1H), 4.52-4.41 (m, 1H), 4.15-4.06 (m, 1H), 3.67-3.60 (m, 1H), 3.38 (t, J=5.6 Hz, 2H), 3.08 (br s. 2H). 2.92-2.79 (m, 2H), 2.70 (t, J=6.4 Hz, 2H), 2.64-2.43 (m, 2H), 2.33 (br s, 1H), 2.11-2.02 (m, 2H), 1.93-1.74 (m, 5H), 1.70-1.51 (m, 2H), 1.36-1.23 (m, 1H); LC-MS (M+H)⁺: 501.4.

### Example 51: 2-(3-phenoxyphenyl)-3-((R)-1-(3-(5,6,7,8-tetrahydro-1, (8-naphthyridin-2-yl)propyl) piperidine-3-carboxamido)propanoic acid (251)

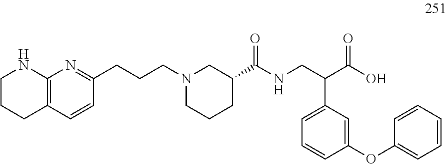 [SMILES: O=C(O)C(CNC(=O)[C@@H]1CCCN(CCCc2ccc3c(n2)NCCC3)C1)c1cccc(Oc2ccccc2)c1]

Following the procedure illustrated in FIG. 4, Scheme 6 and exemplified in Schemes 7, 8 and 9, yielded compound 251 as isomers 251-A and 251-B.

Compound 251-A: ¹H NMR (400 MHz, CDCl₃): δ 10.55 (s, 1H), 8.83 (s, 1H), 7.33-7.27 (m, 3H), 7.27-7.21 (m, 2H), 7.17-7.11 (m, 1H), 7.10 (t, J=4.0 Hz, 1H), 7.08-7.02 (m, 1H), 7.03-6.96 (m, 1H), 6.88-6.82 (m, 1H), 6.28 (d, J=7.2 Hz, 1H). 3.98-3.87 (m, 1H), 3.70-3.55 (m, 2H), 3.42 (t, J=10.8 Hz, 2H), 3.04 (d, J=5.6 Hz, 1H), 2.97-2.65 (m, 5H), 2.55-2.42 (m, 1H), 2.35-2.20 (m, 2H), 2.15-1.95 (m, 3H), 1.93-1.84 (m, 2H), 1.82-1.61 (m, 3H), 1.56-1.41 (m, 2H); LC-MS (M+H)⁺: 543.3.

Compound 251-B. ¹H NMR (400 MHz, CDCl₃): δ 10.75 (s, 1H), 8.72 (s, 1H), 7.32-7.26 (m, 2H), 7.25-7.22 (m, 2H), 7.19-7.15 (m, 1H), 7.14 (t, J=3.6 Hz, 1H), 7.08-7.03 (m, 1H), 7.03-6.96 (m, 2H), 6.86-6.81 (m, 1H), 6.29 (d, J=7.6 Hz, 1H), 3.95-3.84 (m, 1H), 3.80-3.70 (m, 1H), 3.53-3.44 (m, 1H), 3.41 (t, J=11.2 Hz, 2H), 3.17-3.04 (m, 2H), 3.00-2.89 (m, 1H), 2.70 (t, J=12.4 Hz, 2H), 2.65-2.50 (m, 2H), 2.38-2.21 (m, 2H), 2.08-1.99 (m, 2H), 1.92-1.76 (m, 5H), 1.72-1.60 (m, 1H), 1.56-1.41 (m, 2H); LC-MS (M+H)⁺: 543.4.

### Example 52: 3-((R)-1-(3-(5,6,7,8-tetrahydro-1,8-naphthyridin-2-yl)propyl)piperidine-3-carboxamido)-2-(3-(trifluoromethyl)phenyl)propanoic acid (252)

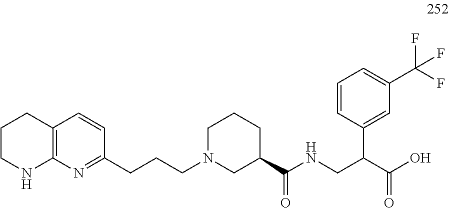 [SMILES: O=C(O)C(CNC(=O)[C@@H]1CCCN(CCCc2ccc3c(n2)NCCC3)C1)c1cccc(C(F)(F)F)c1]

Following the procedure illustrated in FIG. 4, Scheme 6 and exemplified in Schemes 7, 8 and 9, yielded compound 252 as isomers 252-A and 252-B.

Compound 252-A: ¹H NMR (400 MHz, CDCl₃) δ 10.56 (br s, 1H), 8.89 (br s, 1H), 7.68 (s, 1H), 7.56 (d, J=7.6 Hz, 1H), 7.48-7.46 (m, 1H), 7.43-7.39 (m, 1H), 7.25 (s, 1H), 6.32 (d, J=7.2 Hz, 1H), 3.92-3.86 (m, 1H), 3.75 (dd, J₁=8.8 Hz, J₂=2.4 Hz, 1H), 3.68-3.61 (m, 1H), 3.42 (t, J=5.2 Hz, 2H), 3.09 (d, J=11.6 Hz, 1H), 2.98-2.84 (m, 3H), 2.71 (t, J=6.4 Hz, 2H), 2.50 (br s, 1H), 2.42-2.26 (m, 2H), 2.18-2.02 (M, 3H), 1.92-1.72 (m, 5H), 1.61-1.48 (m, 2H): LC-MS (M+H)⁺: 519.3.

Compound 252-B: ¹H NMR (400 MHz, CDCl₃) δ 10.75 (br s, 1H), 8.77 (br s, 1H), 7.70 (m, 1H), 7.61 (d, J=7.6 Hz, 1H), 7.48-7.40 (m, 2H), 7.26-7.21 (m, 1H), 6.32 (d, J=7.2 Hz, 1H), 3.89-3.82 (m, 2H), 3.58-3.50 (m, 1H), 3.42 (t, J=5.2 Hz, 2H), 3.16-2.96 (m, 4H), 2.73-2.58 (m, 4H), 2.31 (br s, 2H), 2.10-1.79 (M, 6H), 1.73-1.43 (m, 3H); LC-MS (M+H)⁺: 519.1.

### Example 53: 3-((R)-1-(3-(5,6,7,8-tetrahydro-1,8-naphthyridin-2-yl)propyl)piperidine-3-carboxamido)-2-(5,6,7,8-tetrahydronaphthalen-1-yl)propanoic acid (253)

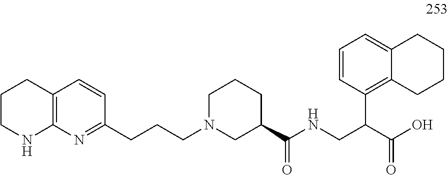 [SMILES: O=C(O)C(CNC(=O)[C@@H]1CCCN(CCCc2ccc3c(n2)NCCC3)C1)c1cccc2c1CCCC2]

Following the procedure illustrated in FIG. 4, Scheme 6 and exemplified in Schemes 7, 8 and 9, yielded compound 253 as isomers 253-A and 253-B.

Compound 253-A: ¹H NMR (400 MHz, DMSO-d₆) δ 8.13 (s, 1H), 7.11 (d, J=7.2 Hz, 1H), 7.06-6.97 (m, 2H), 6.93 (d, J=6.4 Hz, 1H), 6.29 (d, J=7.2 Hz, 1H), 3.96 (t, J=8.0 Hz, 1H), 3.25-3.22 (m, 2H), 2.76-2.66 (m, 4H), 2.62 (t, J=6.0 Hz, 2H), 2.47-2.38 (m, 4H), 2.34-2.23 (m, 2H), 2.22-2.16 (m, 2H), 2.15-1.98 (m, 2H), 1.84-1.60 (m, 9H), 1.57-1.47 (m, 2H), 1.43-1.34 (m, 2H); LC-MS (M+H)⁺: 505.4.

Compound 253-B: ¹H NMR (400 MHz, DMSO-d₆) δ 8.16 (s, 1H), 7.11-6.97 (m, 3H), 6.93 (d, J=6.8 Hz, 1H), 6.88-6.79 (m, 1H), 6.29 (d, J=7.2 Hz, 1H), 3.95 (t, J=6.4 Hz, 1H), 3.25-3.22 (m, 2H), 2.77-2.67 (m, 4H), 2.61 (t, J=6.0 Hz, 2H), 2.48-2.41 (m, 4H), 2.30-1.96 (m, 6H), 1.84-1.62 (m, 9H), 1.58-1.48 (m, 2H), 1.44-1.34 (M, 2H); LC-MS (M+H)⁺: 505.5.

### Example 54: 2-(2,3-dihydro-1H-inden-5-yl)-3-((R)-1-(3-(5,6,7,8-tetrahydro-1,8-naphthyridin-2-yl)propyl)piperidine-3-carboxamido)propanoic acid (254)

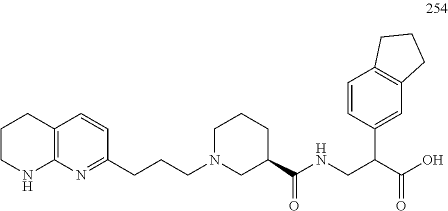 [SMILES: O=C(O)C(CNC(=O)[C@@H]1CCCN(CCCc2ccc3c(n2)NCCC3)C1)c1ccc2c(c1)CCC2]

Following the procedure illustrated in FIG. 4, Scheme 6 and exemplified in Schemes 7, 8 and 9, yielded compound 254 as isomers 254-A and 254-B.

Compound 254-A: ¹H NMR (400 MHz, CDCl₃) δ 10.40 (s, 1H), 8.74 (br s, 1H), 7.24-7.22 (m, 2H), 7.14 (s, 2H), 6.29 (d, J=7.2 Hz, 1H), 4.04-3.90 (m, 1H). 3.64 (dd, J=10.0 Hz, J₂=3.2 Hz, 1H), 3.54-3.47 (m, 1H), 3.40 (t, J=5.6 Hz, 2H), 3.16-3.00 (m, 2H), 2.94-2.74 (m, 6H), 2.70 (t, J=6.0 Hz, 2H), 2.57 (br s, 1H), 2.47-2.15 (m, 3H), 2.07-2.00 (m, 4H), 1.92-1.70 (m, 5H), 1.64-1.47 (m, 2H); LC-MS (M+H)⁺: 491.4.

Compound 254-B: ¹H NMR (400 MHz, CDCl₃) δ 7.27-7.22 (m, 2H), 7.15 (s. 2H), 6.30 (d, J=7.2 Hz, 1H), 3.94-3.79 (M, 1H), 3.74 (dd, J₁=10.8 Hz, J₂=3.6 Hz, 1H), 3.54-3.45 (M, 1H), 3.41 (t, J=5.2 Hz, 2H), 3.20-3.02 (m, 2H), 3.01-2.92 (m, 1H), 2.86 (q, J=8.0 Hz, 4H), 2.82-2.73 (m, 1H), 2.70 (t, J=6.0 Hz, 2H), 2.66-2.48 (m, 2H), 2.47-2.25 (m, 2H), 2.16-1.78 (m, 8H), 1.77-1.46 (m, 3H); LC-MS (M+H)⁺: 491.4.

### Example 55: 2-(3-bromophenyl)-3-((R)-1-(3-(5,6,7,8-tetrahydro-1,8-naphthyridin-2-yl)propyl) piperidine-3-carboxamido)propanoic acid hydrochloride (255)

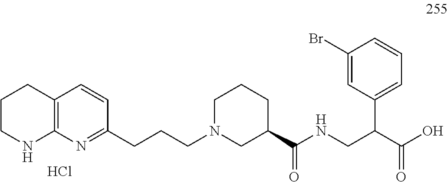 [SMILES: O=C(O)C(CNC(=O)[C@@H]1CCCN(CCCc2ccc3c(n2)NCCC3)C1)c1cccc(Br)c1 ; Cl]

Following the procedure illustrated in FIG. 4, Scheme 6 and exemplified in Schemes 7, 8 and 9, yielded compound 255. ¹H NMR (400 MHz, DMSO-d₆) δ 14.03-13.97 (m, 1H), 10.44-10.38 (m, 1H), 8.29 (s, 1H), 8.00-7.83 (m, 1H), 7.63 (d, J=7.2 Hz, 1H), 7.53-7.43 (m, 2H), 7.36-7.21 (m, 2H), 6.66 (d, J=6.8 Hz, 1H), 3.87-3.55 (m, 4H), 3.35-3.28 (m, 3H), 3.12-2.93 (m, 3H), 2.90-2.82 (m, 1H), 2.79-2.69 (m, 5H), 2.13-2.01 (m, 2H), 1.92-1.56 (m, 6H); LC-MS (M+H)⁺: 531.3.

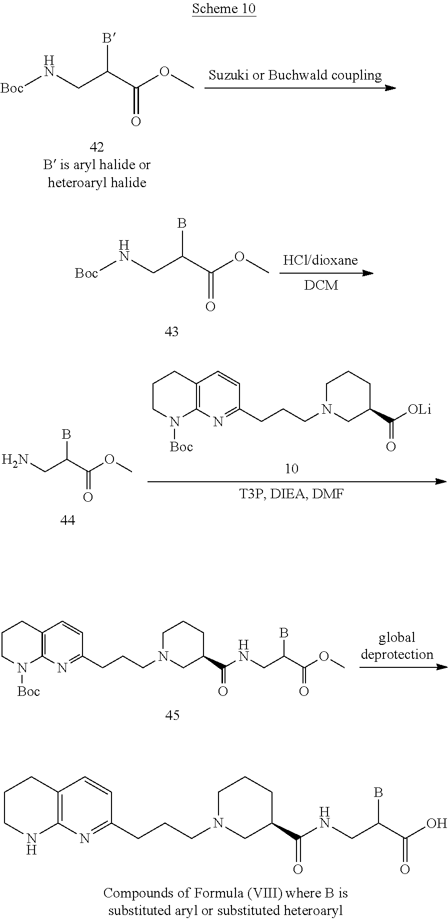

### FIG. 5

FIG. 5, Scheme 10, above illustrates a general synthesis of compounds of Formula (VIII).

### General Procedure for Preparation of Compound 43

To a solution of compound 42 (1.00 eq) and boronic acid/ester (1.20 eq) in dioxane and H₂O was added K₂CO₃ (2.00 eq) and Pd(dppf)Cl₂ (0.100 eq). The mixture was stirred at 90° C. for 12 hrs, filtered and concentrated to give a residue which was purified to obtain compound 43.

### General Procedure for Preparation of Compound 44

To a solution of compound 43 (1.00 eq) in DCM was added HCl/dioxane (9.48 eq) at 0° C. The mixture was stirred at 25° C. for 2 hrs and concentrated to give crude amino ester 44.

### General Procedure for Preparation of Compound 45

To a solution of compound 44 (1.00 eq) and compound 10 (1.01 eq) in DMF (2.00 mL) was added T3P (2.00 eq) and DIEA (3.00 eq) at 0° C. The mixture was stirred at 25° C. for 12 hrs, diluted with saturated NaHCO₃ and extracted with ethyl acetate. The combined organic extracts were washed with brine, dried over Na₂SO₄, filtered and concentrated to give a residue which was purified to obtain compound 45.

### General Procedure for Preparation of a Compound of Formula (VIII)

To a solution of compound 45 (1.00 eq) in H₂O was added HCl/dioxane (25 eq). The mixture was stirred at 60° C. for 3 hrs and was concentrated to give a residue which was purified to obtain a compound of Formula (VIII).

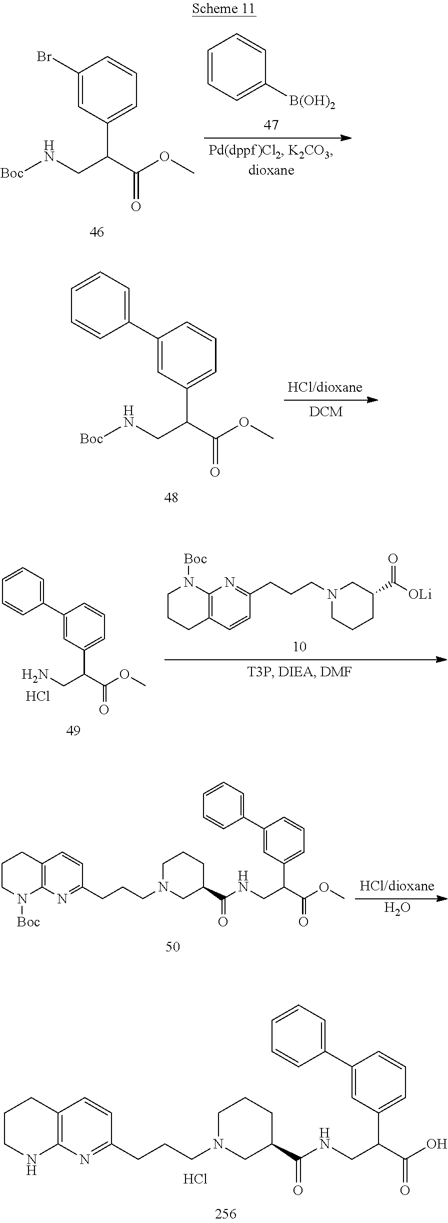

Scheme 11, above, illustrates the synthesis of compound 256 which exemplifies the preparation of compounds of Formula (VIII) shown in FIG. 5. Scheme 10.

### Preparation of Compound 48

To a solution of compound 46 (500 mg, 1.40 mmol, 1.00 eq) and compound 47 (204 mg, 1.67 mmol, 1.20 eq) in dioxane (5.00 mL) and H₂O (0.500 mL) was added K₂CO₃ (386 mg, 2.79 mmol, 2.00 eq) and Pd(dppf)Cl₂ (102 mg, 139 umol, 0.100 eq). The mixture was stirred at 90° C. for 12 hrs until TLC indicated compound 46 was completely consumed and many new spots formed (Petroleum ether: Ethyl acetate=5:1). The reaction mixture was filtered, concentrated to give a residue, which was purified by Prep-TLC to yield compound 48 (250 mg, 703 umol. 50.3% yield) as a yellow oil. ¹H NMR (400 MHz, CDCl₃); S 7.60-7.57 (m, 2H), 7.54-7.51 (m, 1H), 7.48-7.41 (m, 4H), 7.40-7.34 (m, 1H), 7.26-7.24 (m, 1H), 4.92 (brs, 1H), 3.98 (t, J=7.2 Hz, 1H), 3.72 (s, 3H), 3.66-3.53 (m, 2H), 1.43 (s, 9H); LCMS (M-99)⁺: 256.2.

### Preparation of Compound 49

To a solution of compound 48 (150 mg, 422 umol, 1.00 eq) in DCM (2.00 mL) was added HC/dioxane (4 M. 1.00 mL, 9.48 eq) at 0° C. The mixture was stirred at 25° C. for 2 hrs until LC-MS showed that compound 48 was completely consumed and one main peak with the correct mass detected. The reaction mixture was concentrated to give crude compound 49 (130 mg) as a white solid. LCMS (M+H)⁺: 256.2.

### Preparation of Compound 50

To a solution of compound 49 (130 mg, 445 umol, 1.00 eq, HCl) and compound 10 (184 mg, 450 umol, 1.01 eq, Li) in DMF (2.00 mL) was added T3P (567 mg, 891 umol, 529 uL, 50% purity, 2.00 eq) and DIEA (172 mg, 1.34 mmol, 233 uL, 3.00 eq) at 0° C. The mixture was stirred at 25° C. for 12 hrs until LC-MS detected the correct mass. The reaction mixture was diluted with saturated NaHCO₃(10.0 mL) and extracted with ethyl acetate (10.0 mL*3). The combined organic extracts were washed with brine (10.0 mL*2), dried over Na₂SO₄, filtered and concentrated to give a residue which was purified by Prep-TLC (Petroleum ether: Ethyl acetate=0:1) to provide compound 50 (100 mg, 156 umol, 35.0% yield) as a yellow oil. LCMS (M+H)⁺: 641.3; ¹H NMR (400 MHz, CDCl₃) S 7.56-7.35 (m, 9H), 7.24 (s, 2H), 6.80-6.75 (m, 1H), 4.04-3.91 (m, 2H), 3.75 (s, 3H), 3.69-3.61 (m, 4H), 2.72-2.43 (m, 8H), 2.27-2.25 (m, 4H), 1.92-1.81 (m, 6H), 1.51-1.48 (m, 9H).

### Example 56: 2-([1,1′-biphenyl]-3-yl)-3-((R)-1-(3-(5,6,7,8-tetrahydro-1,8-naphthyridin-2-yl)propyl)piperidine-3-carboxamido)propanoic acid (256)

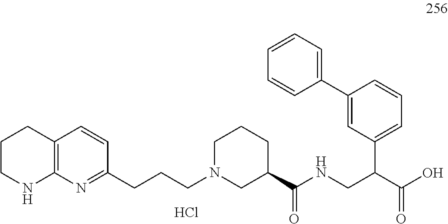 [SMILES: O=C(O)C(CNC(=O)[C@@H]1CCCN(CCCc2ccc3c(n2)NCCC3)C1)c1cccc(-c2ccccc2)c1 ; Cl]

To a solution of compound 50 (100 mg, 156 umol, 1.00 eq) in H₂O (2.00 mL) was added HCl/dioxane (4 M, 1.00 mL, 25.6 eq). The mixture was stirred at 60° C. for 3 hrs until the desired mass was detected by LC-MS, concentrated to give a residue which was purified by Prep-HPLC (column: Phenomenex luna C18 150*25 mm*10 um; mobile phase: [water (0.05% HCl)−ACN]; B %: 11%-41%, 10 min) to provide the racemate 256 (40.0 mg, 71.0 umol, 45.5% yield, HCl) as a yellow oil. The stereoisomers 256-A and 256-B were purified by Prep-SFC (column: DAICEL CHIRALPAK AD (250 mm*30 mm, 10 urn); mobile phase: [0.1% NH₃H₂O IPA]; B %: 60%-60%, 6; 30 min).

Compound 256-A (18.91 mg, 33.50 umol, 47.16% yield, 93.3% purity) was obtained as a yellow gum. ¹H NMR (400 MHz, DMSO-d₆) δ 8.14 (brs, 1H), 7.61 (d, J=7.6 Hz, 2H), 7.53-7.43 (m, 4H), 7.41-7.34 (m, 2H), 7.24 (d, J=7.2 Hz, 1H), 7.10 (d, J=7.2 Hz, 1H), 6.27 (d, J=7.2 Hz, 1H), 3.76 (t, J=7.2 Hz, 1H), 3.60-3.53 (m, 3H), 3.24 (t, J=4.8 Hz, 2H), 2.61 (t, J=6.0 Hz, 2H), 2.43 (t, J=7.2 Hz, 2H), 2.25-2.03 (m, 6H), 1.77-1.62 (m, 4H), 1.47 (d, J=8.0 Hz, 2H), 1.23 (brs, 2H): LC-MS (M+H)⁺: 527.4. SFC chiral purity: 100%.

Compound 256-B (17.00 mg, 31.79 umol. 44.76% yield, 98.5% purity) was obtained as a yellow gum. ¹H NMR (400 MHz, DMSO-d₆) δ 8.12 (brs, 1H), 7.62 (d, J=7.6 Hz, 2H), 7.53-7.44 (m, 4H), 7.42-7.34 (m, 2H), 7.24 (d, J=7.2 Hz, 1H), 7.10 (d, J=7.2 Hz, 1H), 6.27 (d, J=7.2 Hz, 1H), 3.78 (t, J=7.2 Hz, 1H), 3.60-3.53 (m, 1H), 3.51-3.43 (m, 2H), 3.24 (t, J=4.8 Hz. 2H), 2.61 (t, J=6.4 Hz, 2H), 2.43-2.48 (m, 2H), 2.25-2.19 (m, 2H), 2.16-2.01 (m, 4H), 1.77-1.61 (m, 4H), 1.47 (d, J=8.0 Hz, 2H), 1.34 (brs, 2H); LC-MS (M+H)⁺: 527.4; SFC chiral purity: 100%.

### Example 57: 2-(3-(3,5-dimethyl-1H-pyrazol-1-yl)phenyl)-3-((R)-1-(3-(5,6,7,8-tetrahydro-1,8-naphthyridin-2-yl)propyl)piperidine-3-carboxamido)propanoic acid (257)

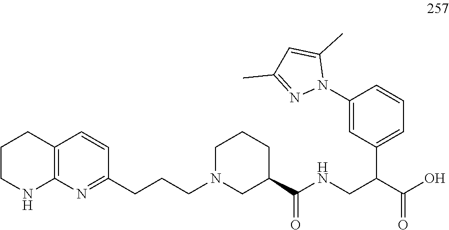 [SMILES: Cc1cc(C)n(-c2cccc(C(CNC(=O)[C@@H]3CCCN(CCCc4ccc5c(n4)NCCC5)C3)C(=O)O)c2)n1]

Following the procedure illustrated in FIG. 5, Scheme 10 and exemplified in Scheme 11, yielded compound 257 as isomers. Compounds 257-A and 257-B were resolved by the procedure of Example 56.

Compound 257-A: ¹H NMR (400 MHz, DMSO-d₆) δ 8.15 (s, 1H), 7.40-7.31 (m, 4H), 7.23 (d, J=7.6 Hz, 1H), 7.12 (d, J=7.2 Hz, 1H), 6.29 (d, 0.1=7.2 Hz, 1H), 6.05 (s, 1H), 3.70 (t, J=6.8 Hz, 1H), 3.24 (t, J=5.2 Hz, 2H), 2.61 (t, J=6.0 Hz, 2H), 2.46-2.44 (m, 4H), 2.26-2.18 (m, 6H), 2.16-2.10 (m, 5H), 2.10-2.00 (m, 2H), 1.77-1.64 (m, 4H), 1.52-1.50 (m, 2H), 1.37 (s, 23; LC-MS (M+H)⁺: 545.5.

Compound 257-B: ¹H NMR (400 MHz, DMSO-d₆+D₂O) δ 7.59 (d, J=7.6 Hz, 1H), 7.44 (t, J=8.0 Hz, 1H), 7.36-7.33 (m, 2H), 7.24 (d, J=7.6 Hz, 1H), 6.61 (d, J=7.6 Hz, 1H), 6.07 (s, 1H), 3.91-3.89 (m, 1H), 3.57-3.44 (m, 2H), 3.43-3.30 (m, 4H), 3.02-3.00 (m, 2H), 2.91-2.81 (m, 1H), 2.78-2.60 (m, 6H), 2.24 (s, 3H), 2.15 (s, 3H), 2.01-1.93 (m, 2H), 1.81-1.78 (m, 2H), 1.68-1.65 (m, 2H), 1.26-1.16 (m, 2H); LC-MS (M+H)⁺: 545.5.

### Example 58: 2-(3-cyclopropylphenyl)-3-((R)-1-(3-(5,6,7,8-tetrahydro-1,8-naphthyridin-2-yl)propyl)piperidine-3-carboxamido)propanoic acid (258)

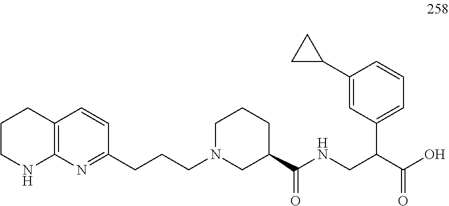 [SMILES: O=C(O)C(CNC(=O)[C@@H]1CCCN(CCCc2ccc3c(n2)NCCC3)C1)c1cccc(C2CC2)c1]

Following the procedure illustrated in FIG. 5, Scheme 10 and exemplified in Scheme 11, yielded compound 258 as isomers. Compound 258-A and compound 258-B were resolved by the procedure of Example 56.

Compound 258-A: ¹H NMR (400 MHz, DMSO-d₆) δ 8.29 (t, J=4.8 Hz, 1H), 7.22-7.12 (m, 2H), 7.01-6.92 (m, 3H), 6.58 (br s, 1H), 6.34 (d, J=6.8 Hz, 1H), 3.71 (t, J=8.4 Hz, 1H), 3.59-3.53 (m, 2H), 3.35-3.26 (m, 4H), 2.98-2.82 (m, 6H), 2.70-7.61 (m, 3H), 1.97-1.85 (m, 3H), 1.75 (s, 5H), 1.43 (br s, 1H), 0.94-0.92 (m, 2H), 0.63 (d, J=4.0 Hz, 2H); LC-MS (M+H)⁺: 491.3

Compound 258-B: ¹H NMR (400 MHz, DMSO-d₆) δ 8.08 (s, 1H), 7.17 (t, J=8.4 Hz, 1H), 7.10 (d, J=7.2 Hz, 1H), 6.99 (d, J=7.6 Hz, 1H), 6.95 (s. 1H), 6.89 (d, J=7.6 Hz, 1H), 6.29 (d, J=7.2 Hz, 1H), 3.64 (t, J=7.2 Hz, 1H), 3.53-3.46 (m, 2H), 3.39-3.32 (m, 2H), 3.24 (t, J=4.8 Hz, 2H), 2.61 (t, J=6.0 Hz, 2H), 2.46-2.41 (m, 2H), 2.25-2.07 (m, 5H), 1.89-1.84 (m, 1H), 1.76-1.67 (m, 4H), 1.51 (d, J=6.0 Hz, 2H), 1.38-1.36 (m, 2H), 0.93-0.88 (m, 2H), 0.64-0.60 (m, 2H); LC-MS: (M+H)⁺: 491.2.

### Example 59: 2-(4-cyclopropylphenyl)-3-((R)-1-(3-(5,6,7,8-tetrahydro-1,8-naphthyridin-2-yl)propyl)piperidine-3-carboxamido)propanoic acid (259)

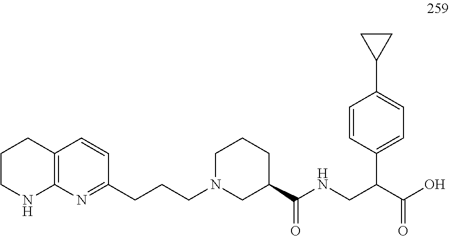 [SMILES: O=C(O)C(CNC(=O)[C@@H]1CCCN(CCCc2ccc3c(n2)NCCC3)C1)c1ccc(C2CC2)cc1]

Following the procedure illustrated in FIG. 5, Scheme 10 and exemplified in Scheme 11, yielded compound 259 as isomers. Compound 259-A and compound 259-B were resolved by the procedure of Example 56.

259-B: ¹H NMR (400 MHz, DMSO-d₆) δ 8.09 (br s, 1H), 7.13-7.07 (m, 3H), 7.00 (d, J=8.4 Hz, 2H), 6.30 (d, J=7.6 Hz, 1H), 3.62 (t, J=8.0 Hz, 2H), 3.23 (t, J=5.2 Hz, 2H), 2.61 (t, J=6.4 Hz, 2H), 2.47-2.40 (m, 3H). 2.35-1.97 (m, 7H), 1.87-1.80 (m, 1H). 1.78-1.65 (m, 4H), 1.55-1.47 (m, 2H), 1.41-1.33 (m, 2H), 0.95-0.87 (m, 2H), 0.65-0.57 (m, 2H): LC-MS (M+H)⁺: 491.4.

259-B: ¹H NMR (400 MHz, DMSO-d₆) δ 8.11 (br s, 1H), 7.15-7.06 (m, 3H), 7.00 (d, J=8.0 Hz, 2H), 6.95-6.85 (m, 1H), 6.29 (d, J=7.6 Hz, 1H), 3.62 (t, J=7.6 Hz, 2H), 3.24 (t, J=4.8 Hz, 2H), 2.61 (t, J=6.0 Hz, 2H), 2.47-2.43 (m, 3H), 2.30-2.14 (m, 4H), 2.12-1.95 (m, 3H), 1.90-1.81 (m, 1H), 1.78-1.65 (m, 4H), 1.77-1.47 (m, 2H), 1.43-1.34 (m, 2H), 0.95-0.87 (m. 2H), 0.65-0.57 (m, 2H); LC-MS (M+H)⁺: 491.6.

### Example 60: 2-(2-cyclopropylphenyl)-3-((R)-1-(3-(5,6,7,8-tetrahydro-1,8-naphthyridin-2-yl)propyl)piperidine-3-carboxamido)propanoic acid (260)

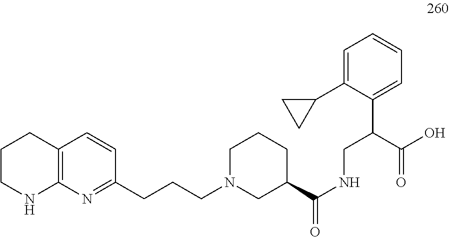 [SMILES: O=C(O)C(CNC(=O)[C@@H]1CCCN(CCCc2ccc3c(n2)NCCC3)C1)c1ccccc1C1CC1]

Following the procedure illustrated in FIG. 5, Scheme 10 and exemplified in Scheme 11, yielded compound 260 as isomers. Compound 260-A and compound 260-B were resolved by the procedure of Example 56.

260-A: ¹H NMR (400 MHz, DMSO-d₆) δ 8.25 (br s, 1H), 7.22-7.06 (m, 4H), 7.03-6.97 (m, 1H), 6.30 (d, J=7.2 Hz, 1H), 4.38 (t, J=6.8 Hz, 1H), 3.55-3.35 (m, 4H), 3.27-3.22 (m, 2H), 3.11-3.05 (m, 1H), 2.87-2.70 (m, 1H), 2.62 (t, J=6.0 Hz, 1H), 2.47-2.30 (m, 4H), 2.13-2.02 (m, 1H), 1.90-1.70 (m, 5H), 1.68-1.56 (m, 2H), 1.55-1.35 (m, 2H), 0.95-0.85 (m, 2H), 0.70-0.54 (m, 2H): LC-MS (M+H)⁺: 491.5.

260-B: ¹H NMR (400 MHz, DMSO-d₆) δ 8.21 (br s, 1H), 7.24-7.06 (m, 4H), 7.03-6.96 (m, 1H), 6.95-6.70 (m, 1H), 6.30 (d, J=7.2 Hz, 1H), 4.37 (t, J=7.6 Hz, 1H), 3.50-3.45 (m, 2H), 3.24 (t, J=5.2 Hz, 2H), 2.69-2.58 (i, 3H), 2.48-2.45 (m, 3H), 2.36-2.15 (m, 5H), 2.09-2.03 (m, 1H), 1.78-1.66 (m, 4H), 1.61-1.51 (m, 2H), 1.48-1.36 (m 0.2H), 0.94-0.85 (m, 2H), 0.70-0.54 (m, 2H); LC-MS (M+H)⁺: 491.5.

The following compounds, set forth in Table 4, were prepared according to the general procedures provided in Schemes 6, and 10, or analogous procedures thereto.

TABLE 4

| Compound | |
|---|---|
| No. | ¹H NMR Data |
| 302 | (400 MHz, DMSO_d₆) δ 8.18-8.16 (m, 1H), 7.45-7.43 (m, 2H), 7.30- |
| | 7.24 (m, 2H), 7.14 (d, J = 6.8 Hz, 1H), 6.32 (d, J = 7.2 Hz, 1H), 3.68 |
| | (t, J = 7.2 Hz, 2H), 3.47 (t, J = 6.0 Hz, 2H), 3.25 (t, J = 5.2 Hz, 4H), |
| | 2.62 (t, J = 6.0 Hz, 2H), 2.30-2.10 (m, 6H), 1.80-1.67 (m, 4H), 1.59- |
| | 1.47 (m, 2H), 1.45-1.35 (m, 2H). |
| 303 | (400 MHz, DMSO_d₆) δ 8.16 (br s, 1H), 7.45-7.42 (m, 2H), 7.29- |
| | 7.24 (m, 2H), 7.15 (d, J = 7.2 Hz, 1H), 6.32 (d, J = 7.2 Hz, 1H), 3.69 (t, |
| | J = 7.2 Hz, 2H), 3.54-3.49 (m, 2H), 3.44-3.39 (m, 2H), 3.25 (t, J = |
| | 5.6 Hz, 2H), 2.62 (t, J = 6.0 Hz, 2H), 2.35-2.10 (m, 6H), 1.79-1.67 |
| | (m, 4H), 1.54-1.46 (m, 2H), 1.43-1.33 (m, 2H). |
| 306 | (400 MHz, DMSO-d₆) δ 8.17 (br s, 1H), 7.66 (d, J = 7.6 Hz, 2H), 7.47 |
| | (t, J = 7.2 Hz, 2H), 7.41-7.34 (m, 3H), 7.13-7.07 (m, 2H), 6.29 (d, J = |
| | 7.2 Hz, 1H), 3.80 (t, J = 7.2 Hz, 1H), 3.63-3.49 (m, 3H), 3.24 (t, J = |
| | 5.2 Hz, 2H), 2.61 (t, J = 6.4 Hz, 2H), 2.45 (t, J = 7.6 Hz, 3H), 2.27- |
| | 2.07 (m, 5H), 1.77-1.62 (m, 4H), 1.52-1.49 (m, 2H), 1.40-1.36 (m, |
| | 2H). |
| 307 | (400 MHz, DMSO-d₆) δ 8.16 (br s, 1H), 7.66 (d, J = 7.6 Hz, 2H), 7.47 |
| | (t, J = 7.2 Hz, 2H), 7.41-7.36 (m, 3H), 7.13-7.06 (m, 2H), 6.28 (d, J = |
| | 7.2 Hz, 1H), 3.81 (t, J = 7.2 Hz, 1H), 3.63-3.43 (m, 3H), 3.24 (t, J = |
| | 5.6 Hz, 2H), 2.61 (t, J = 6.4 Hz, 2H), 2.47-2.36 (m, 3H), 2.30-2.05 |
| | (m, 5H), 1.79-1.60 (m, 4H), 1.48-1.45 (m, 2H), 1.36-1.33 (m, 2H). |
| 308 | (400 MHz, DMSO-d₆) δ 8.14 (br s, 1H), 7.53-7.46 (m, 4H), 7.42- |
| | 7.35 (m, 2H), 7.31-7.22 (m, 2H), 7.10 (d, J = 7.2 Hz, 1H), 6.28 (d, J = |
| | 7.2 Hz, 1H), 3.77 (t, J = 7.6 Hz, 1H), 3.55-3.49 (m, 3H), 3.24 (t, J = |
| | 5.6 Hz, 2H), 2.61 (t, J = 6.0 Hz, 2H), 2.48-2.43 (m, 3H), 2.33-2.07 |
| | (m, 5H), 1.77-1.68 (m, 4H), 1.55-1.48 (m, 2H), 1.41-1.35 (m, 2H). |
| 309 | (400 MHz, DMSO-d₆) δ 8.14 (br s, 1H), 7.53-7.45 (m, 4H), 7.42- |
| | 7.35 (m, 2H), 7.29-7.22 (m, 2H), 7.11 (d, J = 7.2 Hz, 1H), 6.29 (d, J = |
| | 7.2 Hz, 1H), 377 (t, J = 7.2 Hz, 1H), 3.54-3.51 (m, 3H), 3.24 (t, J = |
| | 5.2 Hz, 2H), 2.61 (t, J = 6.4 Hz, 2H), 2.44-2.38 (m, 3H), 2.31-2.08 |
| | (m, 5H), 1.78-1.62 (m, 4H), 1.53-1.42 (m, 2H), 1.40-1.29 (m, 2H). |
| 314 | (400 MHz, CDCl₃) δ 10.43 (s, 1H), 8.99 (br s, 1H), 8.24 (s, 1H), 7.33- |
| | 7.28 (m, 2H), 7.25 (d, J = 7.2 Hz, 1H), 7.14 (dd, J₁ = 6.0 Hz, J₂ = 1.6 |
| | Hz, 1H), 6.31 (d, J = 7.6 Hz, 1H), 4.24 (dd, J₁ = 6.8 Hz, J₂ = 1.6 Hz, |
| | 2H), 4.10-4.01 (m, 2H), 3.75-3.69 (m, 1H), 3.36 (t, J = 5.2 Hz, 2H), |
| | 3.14-3.07 (m, 2H), 2.95-2.93 (m, 1H), 2.86-2.79 (m, 1H), 2.69 (t, J = |
| | 6.0 Hz, 2H), 2.51 (br s, 1H), 2.39-2.24 (m, 2H), 2.17-1.97 (m, |
| | 3H), 1.88-1.79 (m, 5H), 1.57-1.48 (m, 2H), 1.35-1.30 (m, 1H), |
| | 0.58-0.54 (m, 2H), 0.42-0.38 (m, 2H). |
| 315 | (400 MHz, CDCl₃) δ 10.79 (s, 1H), 8.83 (br s, 1H), 8.36 (s, 1H), 7.34- |
| | 7.29 (m, 2H), 7.25 (d, J = 7.2 Hz, 1H), 7.16 (d, J = 6.8 Hz, 1H), 6.32 |
| | (d, J = 7.2 Hz, 1H), 4.25 (d, J = 6.8 Hz, 2H), 4.17 (dd, J₁ = 11.2 Hz, J₂ = |
| | 3.2 Hz, 1H), 4.05-3.99 (m, 1H), 3.62-3.55 (m, 1H), 3.36 (t, J = 5.2 |
| | Hz, 2H), 3.27-3.18 (m, 2H), 3.03 (d, J = 10.0 Hz, 2H), 2.69 (t, J = 6.0 |
| | Hz, 2H), 2.66-2.59 (m, 2H), 2.36-2.29 (m, 2H), 2.09-1.98 (m, 2H), |
| | 1.91-1.81 (m, 4H), 1.77-1.64 (m, 1H), 1.55-1.43 (m, 2H), 1.38- |
| | 1.29 (m, 1H), 0.57-0.52 (m, 2H), 0.42-0.38 (m, 2H). |
| 316 | (400 MHz, DMSO-d₆) δ 8.40 (s, 1H), 8.20 (s, 1H), 7.49 (d, J = 8.8 Hz, |
| | 1H), 7.16 (dd, J₁ = 8.4 Hz, J₂ = 6.8, 1H), 7.10 (d, J = 7.2 Hz, 1H), 7.04- |
| | 6.92 (m, 1H), 6.88 (d, J = 6.8 Hz, 1H), 6.30 (d, J = 7.2 Hz, 1H), 4.28 |
| | (d, J = 7.2 Hz, 2H), 4.02 (t, J = 7.2 Hz, 1H), 3.66-3.50 (m, 6H), 3.23 |
| | (t, J = 5.2 Hz, 2H), 2.61 (t, J = 6.0 Hz, 2H), 2.30-2.09 (m, 5H), 1.78- |
| | 1.64 (m, 4H), 1.56-1.48 (m, 2H), 1.43-1.33 (m, 3H), 0.58-0.53 (m, |
| | 2H), 0.46-0.41 (m, 2H). |
| 317 | (400 MHz, DMSO-d₆) δ 8.40 (s, 1H), 8.17 (s, 1H), 7.49 (d, J = 8.8 Hz, |
| | 1H), 7.18-7.10 (m, 2H), 6.87 (d, J = 6.8 Hz, 1H), 6.30 (d, J = 7.2 Hz, |
| | 1H), 4.27 (d, J = 6.8 Hz, 2H), 4.01 (t, J = 7.2 Hz, 1H), 3.67-3.48 (m, |
| | 6H), 3.23 (t, J = 8.8 Hz, 2H), 2.62 (t, J = 6.0 Hz, 2H), 2.44-2.42 (m, |
| | 2H), 2.22-2.19 (m, 3H), 1.78-1.72 (m, 2H), 1.70-1.65 (m, 2H), |
| | 1.51 (d, J = 8.4 Hz, 2H), 1.42-1.34 (m, 3H), 0.58-0.52 (m, 2H), |
| | 0.45-0.41 (m, 2H). |
| 318 | (400 MHz, CDCl₃) δ 10.44 (s, 1H), 8.99 (br s, 1H), 8.31 (s, 1H), 7.34- |
| | 7.28 (m, 2H), 7.26 (s, 1H), 7.20-7.12 (m, 1H), 6.31 (d, J = 7.2 Hz, |
| | 1H), 4.11-4.03 (m, 2H), 3.79-3.69 (m, 1H), 3.37-3.30 (m, 2H), |
| | 3.16-3.05 (m, 2H), 3.00-2.90 (m, 1H), 2.88-2.79 (m, 1H), 2.69 (t, J = |
| | 6.4 Hz, 2H), 2.54-2.49 (m, 1H), 2.43-2.24 (m, 2H), 2.19-1.96 |
| | (m, 3H), 1.91-1.71 (m, 5H), 1.60-1.45 (m, 2H). |
| 319 | (400 MHz, CDCl₃) δ 10.81 (s, 1H), 8.86-8.83 (m, 1H), 8.45 (s, 1H), |
| | 7.37-7.28 (m, 2H), 7.25 (d, J = 7.2 Hz, 1H), 7.21-7.14 (m, 1H), 6.32 |
| | (d, J = 7.2 Hz, 1H), 4.19 (dd, J₁ = 11.2 Hz, J₂ = 3.6 Hz, 1H), 4.08- |
| | 3.99 (m, 1H), 3.64-3.55 (m, 1H), 3.39-3.32 (m, 2H), 3.29-3.17 (m, |
| | 2H), 3.03 (br d, J = 10.8 Hz, 1H), 2.69 (br t, J = 6.0 Hz, 2H), 2.65- |
| | 2.57 (m, 2H), 2.37-2.25 (m, 2H), 2.10-1.97 (m, 2H), 1.96-1.79 (m, |
| | 5H), 1.78-1.63 (m, 1H), 1.57-1.42 (m, 2H). |
| 320 | (400 MHz, DMSO-d₆) δ 8.37 (br s, 1H), 8.11 (s, 1H), 7.66 (d, J = 7.6 |
| | Hz, 1H), 7.27 (d, J = 6.4 Hz, 1H), 7.17 (d, J = 6.8 Hz, 1H), 7.10 (t, J = |
| | 7.2 Hz, 1H), 6.33 (d, J = 6.8 Hz, 1H), 5.91 (br s, 1H), 4.38 (br s, 1H), |
| | 4.10 (t, J = 7.6 Hz, 1H), 4.01 (t, J = 7.2 Hz, 2H), 3.90-3.86 (m, 2H), |
| | 3.64-3.58 (m, 4H), 3.24 (s, 2H), 2.67-2.55 (m, 4H), 2.40-2.28 (m, |
| | 4H), 2.27-2.19 (m, 2H), 1.74 (s, 4H), 1.60-1.39 (m, 4H). |
| 321 | (400 MHz, DMSO-d₆) δ 8.40 (br s, 1H), 8.11 (s, 1H), 7.66 (d, J = 7.2 |
| | Hz, 1H), 7.28 (d, J = 6.4 Hz, 1H), 7.18 (d, J = 6.8 Hz, 1H), 7.11 (t, J = |
| | 7.2 Hz, 1H), 6.34 (d, J = 7.2 Hz, 1H), 5.89 (br s, 1H), 4.39 (br s, 1H), |
| | 4.08-3.97 (m, 4H), 3.94-3.85 (m, 3H), 3.65-3.62 (m, 2H), 3.24 (s, |
| | 2H), 2.63 (s, 2H), 2.59-2.53 (m, 2H), 2.43-2.29 (m, 4H), 2.24-2.15 |
| | (m, 2H), 1.81-1.66 (m, 4H), 1.62-1.37 (m, 4H). |
| 322 | (400 MHz, DMSO-d₆) δ 8.39 (br s, 1H), 8.11 (s, 1H), 7.66 (d, J = 7.6 |
| | Hz, 1H), 7.28 (d, J = 6.4 Hz, 1H), 7.21 (d, J = 6.0 Hz, 1H), 7.10 (t, J = |
| | 7.2 Hz, 1H), 6.36 (d, J = 7.2 Hz, 1H), 5.92 (br s, 1H), 4.41 (br s, 1H), |
| | 4.10-4.01 (m, 3H), 3.98-3.89 (m, 4H), 3.57 (s, 2H), 3.24 (s, 2H), |
| | 2.63 (br s, 3H), 2.44-2.30 (m, 5H), 2.25 (s, 2H), 1.80-1.66 (m, 4H), |
| | 1.57-1.38 (m, 4H). |
| 323 | (400 MHz, DMSO-d₆) δ 8.38 (s, 1H), 8.11 (s, 1H), 7.66 (d, J = 8.0 Hz, |
| | 1H), 7.28 (d, J = 6.8 Hz, 1H), 7.20 (d, J = 6.4 Hz, 1H), 7.10 (t, J = 7.6 |
| | Hz, 1H), 6.36 (d, J = 6.8 Hz, 1H), 5.93 (br s, 1H), 4.43 (br s, 1H), 4.15 |
| | (t, J = 7.6 Hz, 1H), 4.06-3.97 (m, 3H), 3.93-3.86 (m, 3H), 3.58 (s, |
| | 2H), 3.24 (s, 2H), 2.63 (s, 2H), 2.43-2.32 (m, 6H), 2.26 (s, 2H), 1.79- |
| | 1.66 (m, 4H), 1.57-1.37 (m, 4H). |
| 324 | (400 MHz, DMSO-d₆) δ 11.57 (s, 1H), 8.12 (br s, 1H), 8.06 (d, J = 2.0 |
| | Hz, 1H), 7.81 (d, J = 1.6 Hz, 1H), 7.43 (t, J = 3.2 Hz, 1H), 7.09 (d, J = |
| | 7.2 Hz, 2H), 7.04-6.92 (m, 1H), 6.40 (dd, J₁ = 3.2 Hz, J₂ = 1.6 Hz, |
| | 1H), 6.29 (d, J = 7.2 Hz, 1H), 3.75 (t, J = 7.2 Hz, 1H), 3.23 (t, J = 5.2 |
| | Hz, 2H), 2.61 (t, J = 6.0 Hz, 2H), 2.48-2.41 (m, 5H), 2.26-2.11 (m, |
| | 4H), 2.04-1.95 (m, 2H), 1.77-1.60 (m, 4H), 1.55-1.47 (m, 2H), |
| | 1.39-1.32 (m, 2H). |
| 325 | (400 MHz, DMSO-d₆) δ 11.53 (s, 1H), 8.14-8.03 (m, 2H), 7.80 (d, J = |
| | 1.6 Hz, 1H), 7.43-7.37 (m, 2H), 7.09 (d, J = 7.2 Hz, 1H), 6.96 (d, J = |
| | 8.0 Hz, 1H), 6.38 (t, J = 2.0 Hz, 1H), 6.28 (d, J = 7.2 Hz, 1H), 3.71- |
| | 3.67 (m, 1H), 3.23 (t, J = 5.2 Hz, 2H), 2.61 (t, J = 6.0 Hz, 2H), 2.45- |
| | 2.40 (m, 5H), 2.25-2.14 (m, 4H), 2.03-1.95 (m, 2H), 1.76-1.72 (m, |
| | 2H), 1.68-1.64 (m, 2H), 1.55-1.47 (m, 2H), 1.36-1.30 (m, 2H). |
| 326 | (400 MHz, DMSO-d₆) δ 8.13 (br s, 1H), 8.10 (d, J = 2.0 Hz, 1H), 7.83 |
| | (d, J = 2.0 Hz, 1H), 7.59 (d, J = 3.2 Hz, 1H), 7.10 (d, J = 7.2 Hz, 1H), |
| | 7.06-6.95 (m, 1H), 6.42 (d, J = 3.6 Hz, 1H), 6.29 (d, J = 7.2 Hz, 1H), |
| | 4.08 (d, J = 7.2 Hz, 2H), 3.78 (t, J = 7.2 Hz, 1H), 3.63-3.44 (m, 3H), |
| | 3.23 (t, J = 5.2 Hz, 2H), 2.61 (t, J = 6.4 Hz, 2H), 2.47-2.40 (m, 3H), |
| | 2.30-2.15 (m, 3H), 2.10-1.95 (m, 2H), 1.80-1.62 (m, 4H), 1.56- |
| | 1.47 (m, 2H), 1.43-1.32 (m, 2H), 1.30-1.21 (m, 1H), 0.50-0.43 (m, |
| | 2H), 0.42-0.35 (m, 2H). |
| 327 | (400 MHz, DMSO-d₆) δ 8.12 (br s, 1H), 8.09 (d, J = 2.0 Hz, 1H), 7.82 |
| | (d, J = 2.0 Hz, 1H), 7.59 (d, J = 3.6 Hz, 1H), 7.10 (d, J = 7.2 Hz, 1H), |
| | 6.42 (d, J = 3.2 Hz, 1H), 6.29 (d, J = 7.2 Hz, 1H), 4.08 (d, J = 6.8 Hz, |
| | 2H), 3.79 (t, J = 7.6 Hz, 1H), 3.65-3.56 (m, 1H), 3.48-3.37 (m, 1H), |
| | 3.23 (t, J = 4.8 Hz, 2H), 2.61 (t, J = 6.0 Hz, 2H), 2.48-2.38 (m, 4H), |
| | 2.30-2.13 (m, 4H), 2.10-2.03 (m, 1H), 1.78-1.71 (m, 2H), 1.70- |
| | 1.62 (m, 2H), 1.54-1.45 (m, 2H), 1.38-1.32 (m, 2H), 1.26-1.22 (m, |
| | 1H), 0.50-0.42 (m, 2H), 0.41-0.36 (m, 2H). |
| 340 | (400 MHz, CDCl₃) δ 10.74 (s, 1H), 8.60 (s, 1H), 7.61-7.56 (m, 2H), |
| | 7.50 (d, J = 7.4 Hz, 1H), 7.47-7.40 (m, 2H), 7.35-7.27 (m, 3H), 7.27- |
| | 7.19 (m, 2H), 6.29 (d, J = 7.3 Hz, 1H), 3.93 (dd, J₁ = 10.4 Hz, J₂ = |
| | 3.2 Hz, 1H), 3.90-3.82 (m, 1H), 3.43-3.34 (m, 3H), 3.21-3.13 (m, |
| | 1H), 3.08-2.94 (m, 2H), 2.68 (t, J = 6.2 Hz, 2H), 2.63-2.53 (m, 1H), |
| | 2.45 (s, 1H), 2.26-2.25 (m, 2H), 2.06-1.94 (m, 2H), 1.89-1.76 (m, |
| | 5H), 1.74-1.62 (m, 1H), 1.52-1.36 (m, 2H). |
| 341 | (400 MHz, CDCl₃) δ 10.48 (s, 1H), 8.82 (s, 1H), 7.57-7.52 (m, 2H), |
| | 7.44-7.36 (m, 3H), 7.33-7.27 (m, 2H), 7.26-7.21 (m, 3H), 6.28 (d, |
| | J = 7.3 Hz, 1H), 3.85 (dd, J₁ = 8.4 Hz, J₂ = 2.4 Hz, 1H), 3.82-3.76 |
| | (m, 1H), 3.57-3.50 (m, 1H), 3.41 (t, J = 5.4 Hz, 2H), 3.11 (d, J = 11.3 |
| | Hz, 1H), 2.96-2.79 (m, 3H), 2.69 (t, J = 6.1 Hz, 2H), 2.44 (s, 1H), |
| | 2.39-2.21 (m, 2H), 2.11 (d, J = 8.9 Hz, 1H), 2.01-1.83 (m, 4H), 1.81- |
| | 1.62 (m, 2H), 1.61-1.50 (m, 1H), 1.48-1.36 (m, 2H). |
| 342 | (400 MHz, DMSO-d₆) δ 8.25 (s, 1H), 7.66-7.60 (m, 4H), 7.49-7.42 |
| | (m. 2H), 7.39-7.32 (m, 3H), 7.09 (d, J = 7.2 Hz, 1H), 6.90-6.70 (m, |
| | 1H), 6.28 (d, J = 7.6 Hz, 1H), 3.77 (t, J = 7.2 Hz, 1H), 3.63-3.39 (m, |
| | 4H), 3.24 (t, J = 5.2 Hz, 2H), 2.88-2.73 (m, 2H), 2.64-2.57 (m, 3H), |
| | 2.47-2.42 (m, 4H), 1.83-1.77 (m, 2H), 1.76-1.72 (m, 2H), 1.67- |
| | 1.60 (m, 2H), 1.56-1.41 (m, 2H). |
| 343 | (400 MHz, DMSO-d₆) δ 8.16 (s, 1H), 7.66-7.57 (m, 4H), 7.48-7.42 |
| | (m, 2H), 7.38-7.31 (m, 3H), 7.08 (d, J = 7.2 Hz, 1H), 6.28 (d, J = 7.6 |
| | Hz, 1H), 3.72 (t, J = 7.2 Hz, 1H), 3.58-3.53 (m, 2H), 3.47-3.41 (m, |
| | 2H), 3.23 (t, J = 5.2 Hz, 2H), 2.60 (t, J = 6.0 Hz, 2H), 2.46-2.40 (m, |
| | 2H), 2.32-2.07 (m, 5H), 1.80-1.63 (m, 4H), 1.57-1.47 (m, 2H), |
| | 1.45-1.33 (m, 2H). |
| 538 | (400 MHz, DMSO-d₆) δ 14.32 (s, 1H), 11.05-10.73 (m, 1H), 8.44- |
| | 8.31 (m, 1H), 8.06 (s, 1H), 7.62 (d, J = 7.2 Hz, 1H), 7.18-7.06 (m, |
| | 2H), 6.96 (d, J = 6.8 Hz, 1H), 6.67 (dd, J₁ = 7.6 Hz, J₂ = 1.6 Hz, 1H), |
| | 4.83-4.62 (m, 2H), 4.06-3.96 (m, 1H), 3.57-3.48 (m, 2H), 3.47- |
| | 3.41 (m, 5H), 3.37-3.32 (m, 2H), 3.18 (s, 3H), 3.08-2.97 (m, 2H), |
| | 2.90-2.70 (m, 7H), 2.20-2.07 (m, 2H), 2.05-1.75 (m, 5H), 1.45- |
| | 1.30 (m, 1H), 1.25-1.14 (m, 6H). |
| 560 | (400 MHz, CDCl₃) δ 10.51 (s, 1H), 8.80 (s, 1H), 7.24 (d, J = 7.2 Hz, |
| | 1H), 7.14-7.11 (m, 1H), 7.10-7.05 (m, 2H), 6.29 (d, J = 7.6 Hz, 1H), |
| | 3.95-3.88 (m, 1H), 3.76 (dd, J₁ = 9.6 Hz, J₂ = 2.8 Hz, 1H), 3.54- |
| | 3.47 (m, 1H), 3.41 (t, J = 5.2 Hz, 2H), 3.14-2.98 (m, 4H), 2.96-2.88 |
| | (m, 3H), 2.86-2.78 (m, 1H), 2.70 (t, J = 6.0 Hz, 2H), 2.51 (br s, 1H), |
| | 2.43-2.25 (m, 2H), 2.21-1.95 (m, 5H), 1.92-1.67 (m, 5H), 1.60- |
| | 1.42 (m, 2H). |
| 561 | (400 MHz, CDCl₃) δ 10.86 (br s, 1H), 8.73 (br s, 1H), 7.23 (d, J = 7.6 |
| | Hz, 1H), 7.16-7.11 (m, 1H), 7.11-7.08 (m, 2H), 6.30 (d, J = 7.6 Hz, |
| | 1H), 3.92-3.83 (m, 2H), 3.40 (t, J = 5.6 Hz, 2H), 3.37-3.28 (m, 1H), |
| | 3.27-2.96 (m, 5H), 2.92 (t, J = 7.6 Hz, 2H), 2.70 (t, J = 6.4 Hz, 2H), |
| | 2.63-2.50 (m, 2H), 2.29 (t, J = 5.2 Hz, 2H), 2.10-1.95 (m, 4H), 1.92- |
| | 1.65 (m, 6H), 1.57-1.41 (m, 2H). |
| 565 | (400 MHz, DMSO-d₆) δ 8.05 (br s, 1H), 7.12-7.05 (m, 2H), 6.89 (br |
| | s, 1H), 6.55-6.44 (m, 3H), 6.28 (d, J = 7.2 Hz, 1H), 3.97-3.88 (m, |
| | 4H), 3.63-3.57 (m, 4H), 3.30-3.21 (m, 8H), 2.67-2.59 (m, 2H), |
| | 2.34-2.29 (m, 1H), 2.28-2.18 (m, 3H), 2.07-1.97 (m, 2H), 1.92- |
| | 1.82 (m, 2H), 1.77-1.65 (m, 4H), 1.52 (d, J = 5.6 Hz, 2H), 1.41-1.32 |
| | (m, 2H). |
| 566 | (400 MHz, DMSO-d₆) δ 8.06 (br s, 1H), 7.12-7.05 (m, 2H), 6.92 (br |
| | s, 1H), 6.56-6.45 (m, 3H), 6.28 (d, J = 7.2 Hz, 1H), 3.96-3.88 (m, |
| | 4H), 3.64-3.59 (m, 4H), 3.39-3.34 (m, 4H), 3.27-3.21 (m, 4H), |
| | 2.61 (t, J = 6.0 Hz, 2H), 2.34-2.11 (m, 4H), 2.08-1.96 (m, 2H), 1.93- |
| | 1.81 (m, 2H), 1.75-1.64 (m, 4H), 1.56-1.48 (m, 2H), 1.42-1.34 |
| | (m, 2H). |
| 594 | (400 MHz, DMSO-d₆) δ 8.10 (s, 1H), 7.16-7.08 (m, 2H), 6.88 (s, |
| | 1H), 6.80 (d, J = 8.4 Hz, 1H), 6.76 (s, 1H), 6.68 (d J = 7.6 Hz, 1H), |
| | 6.29 (d, J = 7.2 Hz, 1H), 3.73 (t, J = 4.8 Hz, 2H), 3.62 (t, J = 7.6 Hz, |
| | 1H), 3.44 (t, J = 6.4 Hz, 3H), 3.24 (t, J = 5.2 Hz, 3H), 3.00 (t, J = 4.4 |
| | Hz, 2H), 2.89 (s, 2H), 2.61 (t, J = 5.6 Hz, 2H), 2.44 (s, 1H), 2.33-2.05 |
| | (m, 6H), 1.76-1.66 (m, 4H), 1.54-1.52 (m, 2H), 1.41-1.38 (m, 2H), |
| | 1.22 (s, 6H). |
| 595 | (400 MHz, DMSO-d₆) δ 8.08 (s, 1H), 7.16-7.09 (m, 2H), 7.00 (br s, |
| | 1H), 6.80-6.78 (m, 2H), 6.68 (d, J = 7.6 Hz, 1H), 6.29 (d, J = 7.2 Hz, |
| | 1H), 3.73 (t, J = 4.8 Hz, 2H), 3.62 (t, J = 7.6 Hz, 1H), 3.53-3.46 (m, |
| | 2H), 3.41-3.34 (m, 2H), 3.25-3.23 (m, 2H), 3.00 (t, J = 4.8 Hz, 2H), |
| | 2.89 (s, 2H), 2.61 (t, J = 6.0 Hz, 2H), 2.44-2.40 (m, 1H), 2.28-2.07 |
| | (m, 6H), 1.78-1.66 (m, 4H), 1.53-1.51 (m, 2H), 1.41-1.37 (m, 2H), |
| | 1.22 (s, 6H). |
| 596 | (400 MHz, DMSO-d₆) δ 8.09 (s, 1H), 7.20 (t, J = 8.0 Hz, 1H), 7.10 (d, |
| | J = 7.2 Hz, 1H), 7.00-6.94 (m, 4H), 6.29 (d, J = 7.2 Hz, 1H), 3.69- |
| | 3.62 (m, 4H), 3.46-3.41 (m, 3H), 3.38 (s, 2H), 3.24 (t, J = 5.2 Hz, |
| | 2H), 3.03-2.96 (m, 2H), 2.61 (t, J = 6.0 Hz, 2H), 2.46-2.44 (m, 2H), |
| | 2.26-2.01 (m, 5H), 1.78-1.66 (m, 4H), 1.53-1.52 (m, 2H), 1.41- |
| | 1.37 (m, 2H), 0.93 (s, 6H). |
| 597 | (400 MHz, DMSO-d₆) δ 8.09 (s, 1H), 7.21 (t, J = 8.0 Hz, 1H), 7.10 (d, |
| | J = 7.6 Hz, 1H), 7.00-6.96 (m, 3H), 6.29 (d, J = 7.2 Hz, 1H), 3.69- |
| | 3.64 (m, 5H), 3.41-3.39 (m, 2H), 3.38 (s, 2H), 3.24 (t, J = 4.4 Hz, |
| | 2H), 3.02-2.99 (m, 2H), 2.61 (t, J = 6.0 Hz, 2H), 2.45-2.41 (m, 2H), |
| | 2.26-2.08 (m, 5H), 1.76-1.67 (m, 4H), 1.51-1.49 (m, 2H), 1.36- |
| | 1.35 (m, 2H), 0.93 (s, 6H). |
| 598 | (400 MHz, CDCl₃) δ 10.43 (br s, 1H), 8.80 (br s, 1H), 7.23 (d, J = 7.2 |
| | Hz, 1H), 7.15 (t, J = 7.6 Hz, 1H), 6.92 (s, 1H), 6.82 (d, J = 7.6 Hz, |
| | 1H), 6.75 (dd, J₁ = 8.0 Hz, J₂ = 2.0 Hz, 1H), 6.29 (d, J = 7.2 Hz, 1H), |
| | 4.02-4.00 (m, 1H), 3.59-3.49 (m, 2H), 3.40 (t, J = 5.2 Hz, 2H), 3.08- |
| | 3.06 (m, 4H), 2.90-2.89 (m, 1H), 2.80-2.75 (m, 3H), 2.69 (t, J = |
| | 6.4 Hz, 2H), 2.54 (s, 1H), 2.37-2.22 (m, 2H), 2.15-1.98 (m, 3H), |
| | 1.88-1.67 (m, 7H), 1.52-1.45 (m, 2H), 1.33 (t, J = 6.4 Hz, 2H), 1.00 |
| | (s, 6H). |
| 599 | (400 MHz, CDCl₃) δ 10.74 (br s, 1H), 8.70 (br s, 1H), 7.22 (d, J = 7.2 |
| | Hz, 1H), 7.15 (t, J = 8.0 Hz, 1H), 6.94 (s, 1H), 6.85 (d, J = 7.6 Hz, |
| | 1H), 6.75 (dd, J₁ = 8.0 Hz, J₂ = 2.0 Hz, 1H), 6.29 (d, J = 7.2 Hz, 1H), |
| | 3.96-3.94 (m, 1H), 3.71 (dd, J₁ = 10.8 Hz, J₂ = 3.6 Hz, 1H), 3.41- |
| | 3.38 (m, 3H), 3.09-3.06 (m, 5H), 2.81 (s, 2H), 2.70-2.67 (m, 4H), |
| | 2.28-2.27 (m, 2H), 2.02 (t, J = 13.2 Hz, 2H), 1.87-1.85 (m, 5H), |
| | 1.69-1.68 (m, 3H), 1.50-1.45 (m, 2H), 1.33 (t, J = 6.0 Hz, 2H), 0.99 |
| | (s, 6H). |
| 600 | (400 MHz, CDCl₃) δ 10.42 (s, 1H), 8.79 (s, 1H), 7.23 (d, J = 7.2 Hz, |
| | 1H), 7.17 (t, J = 7.6 Hz, 1H), 6.97 (s, 1H), 6.85 (d, J = 7.2 Hz, 1H), |
| | 6.81 (d, J = 8.0 Hz, 1H), 6.29 (d, J = 7.2 Hz, 1H), 4.05-3.96 (m, 1H), |
| | 3.64-3.59 (m, 1H), 3.56-3.47 (m, 1H), 3.40 (t, J = 5.2 Hz, 2H), 3.18- |
| | 3.14 (m, 4H), 3.12-3.01 (m, 2H), 2.93-2.85 (m, 1H), 2.83-2.75 |
| | (m, 1H), 2.70 (t, J = 6.0 Hz, 2H), 2.54 (s, 1H), 2.36-2.21 (m, 2H), |
| | 2.16-1.96 (m, 3H), 1.91-1.74 (m, 5H), 1.54-1.46 (m, 6H), 0.97 (s, 6H). |
| 601 | (400 MHz, CDCl₃) δ 10.74 (s, 1H), 8.69 (s, 1H), 7.23 (d, J = 7.6 Hz, |
| | 1H), 7.17 (t, J = 7.6 Hz, 1H), 7.00 (s, 1H), 6.88 (d, J = 7.6 Hz, 1H), |
| | 6.81 (d, J = 7.2 Hz, 1H), 6.30 (d, J = 7.2 Hz, 1H), 3.98-3.88 (m, 1H), |
| | 3.72 (dd, J₁ = 10.8 Hz, J₂ = 3.2, 1H), 3.47-3.37 (m, 3H), 3.20-3.11 |
| | (m, 6H), 3.03-2.96 (m, 1H), 2.69 (t, J = 6.0 Hz, 2H), 2.65-2.53 (m, |
| | 2H), 2.29-2.27 (m, 2H), 2.03 (d, J = 12.0 Hz, 2H), 1.91-1.80 (m, |
| | 5H), 1.68 (d, J = 12.4 Hz, 1H), 1.53-1.46 (m, 6H), 0.97 (s, 6H). |
| 602 | (400 MHz, CDCl₃) δ 10.47 (br s, 1H), 8.83-8.81 (m, 1H), 7.24 (d, J = |
| | 7.2 Hz, 1H), 7.19 (t, J = 8.0 Hz, 1H), 6.93-6.88 (m, 2H), 6.76 (dd, J₁ = |
| | 8.0 Hz, J₂ = 2.0 Hz, 1H), 6.29 (d, J = 7.2 Hz, 1H), 4.03-3.97 (m, |
| | 1H), 3.85-3.71 (m, 2H), 3.65-3.36 (m, 6H), 3.16-3.00 (m, 2H), |
| | 2.93-2.74 (m, 2H), 2.70 (t, J = 6.0 Hz, 2H), 2.54 (br s, 1H), 2.47- |
| | 2.38 (m, 2H), 2.35-2.20 (m, 2H), 2.17-1.96 (m, 3H), 1.91-1.84 (m, |
| | 2H), 1.83-1.66 (m, 3H), 1.60-1.41 (m, 2H), 1.25 (d, J = 6.0 Hz, 6H). |
| 603 | (400 MHz, DMSO-d₆) δ 8.30 (br s, 1H), 8.04 (br s, 1H), 7.15-7.07 |
| | (m, 2H), 6.78-6.76 (m, 2H), 6.67 (d, J = 7.6 Hz, 1H), 6.28 (d, J = 7.2 |
| | Hz, 1H), 4.07-4.00 (m, 3H), 3.58 (t, J = 7.2 Hz, 2H), 3.51-3.43 (m, |
| | 2H), 3.41-3.32 (m, 2H), 3.23 (t, J = 5.2 Hz, 2H), 3.13 (dd, J₁ = 11.6 |
| | Hz, J₂ = 3.2 Hz, 2H), 2.79 (dd, J₁ = 11.6 Hz, J₂ = 6.0 Hz, 2H), 2.61 (t, |
| | J = 6.0 Hz, 2H), 2.44-2.40 (m, 1H), 2.30-2.16 (m, 3H), 2.13-2.02 |
| | (m, 1H), 1.82-1.60 (m, 4H), 1.58-1.45 (m, 2H), 1.42-1.31 (m, 2H), |
| | 1.25-1.17 (m, 6H). |
| 604 | (400 MHz, CDCl₃) δ 10.80 (br s, 1H), 8.73-8.70 (m, 1H), 7.26-7.17 |
| | (m, 2H), 6.96-6.91 (m, 2H), 6.76 (dd, J₁ = 8.0 Hz, J₂ = 2.0 Hz, 1H), |
| | 6.30 (d, J = 7.2 Hz, 1H), 3.97-3.88 (m, 1H), 3.84-3.70 (m, 3H), 3.53- |
| | 3.36 (m, 5H), 3.26-3.11 (m, 2H), 3.00 (br d, J = 10.8 Hz, 1H), 2.70 |
| | (t, J = 6.0 Hz, 2H), 2.65-2.51 (m, 2H), 2.44-2.38 (m, 2H), 2.34- |
| | 2.21 (m, 2H), 2.10-1.95 (m, 2H), 1.92-1.76 (m, 5H), 1.75-1.61 (m, |
| | 1H), 1.54-1.39 (m, 2H), 1.25 (d, J = 6.0 Hz, 6H). |
| 605 | (400 MHz, DMSO-d₆) δ 8.15-8.11 (m, 1H), 7.16-7.08 (m, 2H), 6.99- |
| | 6.85 (m, 1H), 6.80-6.75 (m, 2H), 6.67 (d, J = 7.6 Hz, 1H), 6.29 (d, J = |
| | 7.2 Hz, 1H), 4.05-3.99 (m, 2H), 3.63 (t, J = 7.2 Hz, 2H), 3.52-3.46 |
| | (m, 3H), 3.42-3.37 (m, 2H), 3.26-3.22 (m, 2H), 3.14 (dd, J₁ = 11.6 |
| | Hz, J₂ = 2.8 Hz, 2H), 2.80 (dd, J₁ = 12.0 Hz, J₂ = 6.4 Hz, 2H), 2.61 (t, |
| | J = 6.0 Hz, 2H), 2.46-2.40 (m, 2H), 2.36-2.30 (m, 3H), 1.78-1.69 |
| | (m, 4H), 1.59-1.49 (m, 2H), 1.44-1.34 (m, 2H), 1.19 (d, J = 6.4 Hz, |
| | 6H). |
| 619 | (400 MHz, DMSO-d6) δ 8.44 (s, 1H), 8.06 (s, 1H), 7.56 (d, J = 8.4 |
| | Hz, 1H), 7.10 (t, J = 8.0 Hz, 2H), 7.01-6.95 (m, 1H), 6.29 (d, J = 7.2 |
| | Hz, 1H), 5.25 (t, J = 7.6 Hz, 1H), 4.31 (t, J = 6.0 Hz, 1H), 3.79-3.72 |
| | (m, 2H), 3.59-3.55 (m, 2H), 3.26-3.23 (m, 3H), 2.85-2.74 (m, 2H), |
| | 2.62 (t, J = 5.2 Hz, 2H), 2.43-2.40 (m, 2H), 2.34-2.23 (m, 6H), 1.77- |
| | 1.64 (m, 4H), 1.52-1.46 (m, 2H), 1.40-1.33 (m, 2H), 1.25-1.16 (m, 2H) |
| 620 | (400 MHz, DMSO-d6) δ 8.75 (d, J = 2.0 Hz, 1H), 8.44 (d, J = 1.6 |
| | Hz, 1H), 8.19 (br s, 1H), 7.91 (s, 1H), 7.70 (br d, J = 7.6 Hz, 2H), 7.50 |
| | (br t, J = 7.6 Hz, 2H), 7.45-7.39 (m, 1H), 7.13 (br d, J = 7.2 Hz, 1H), |
| | 6.29 (d, J = 7.2 Hz, 1H), 3.83 (br t, J = 7.2 Hz, 1H), 3.69-3.56 (m, |
| | 2H), 3.24 (br s, 2H), 2.61 (br t, J = 6.0 Hz, 2H), 2.47-2.36 (m, 4H), |
| | 2.29-2.09 (m, 5H), 1.78-1.63 (m, 4H), 1.49-1.31 (m, 4H) |
| 621 | (400 MHz, CDCl3) δ 10.74 (s, 1H), 8.74 (s, 1H), 7.71 (d, J = 7.6 |
| | Hz, 1H), 7.56-7.50 (m, 1H), 7.44 (t, J = 6.8 Hz, 2H), 7.40 (s, 1H), |
| | 7.38-7.31 (m, 2H), 7.24-7.16 (m, 2H), 6.28 (d, J = 7.2 Hz, 1H), 3.98- |
| | 3.89 (m, 1H), 3.86-3.80 (m, 1H), 3.54-3.45 (m, 1H), 3.41 (t, J = |
| | 5.6 Hz, 2H), 3.18-3.08 (m, 2H), 2.95 (d, J = 10.4 Hz, 1H), 2.69 (t, J = |
| | 6.2 Hz, 2H), 2.63-2.52 (m, 2H), 2.33-2.21 (m, 2H), 2.06-1.97 (m, |
| | 2H), 1.92-1.83 (m, 4H), 1.81 (s, 1H), 1.71-1.58 (m, 1H), 1.52-1.40 |
| | (m, 2H) |
| 622 | (400 MHz, DMSO-d6). δ 8.18 (s, 1H), 7.51-7.33 (m, 7H), 7.15 (t, J = |
| | 9.2 Hz, 2H), 6.30 (d, J = 7.2 Hz, 1H), 3.91 (t, J = 7.2 Hz, 1H), 3.57- |
| | 3.44 (m, 3H), 3.24 (t, J = 5.6 Hz, 2H), 2.67-2.60 (m, 3H), 2.49-2.43 |
| | (m, 2H), 2.29-2.17 (m, 5H), 1.77-1.63 (m, 4H), 1.53-1.39 (m, 4H). |
| 623 | (400 MHz, DMSO-d6) δ 8.44 (s, 1H), 8.07 (br s, 1H), 7.56 (d, J = 8.4 |
| | Hz, 1H), 7.10 (t, J = 6.4 Hz, 2H), 6.98 (t, J = 7.6 Hz, 1H), 6.29 (d, J = |
| | 7.2 Hz, 1H), 5.31-5.19 (m, 1H), 4.31 (t, J = 7.2 Hz, 1H), 3.77-3.72 |
| | (m, 2H), 3.60-3.54 (m, 3H), 3.24 (br s, 2H), 2.85-2.74 (m, 2H), 2.62 |
| | (t, J = 6.0 Hz, 2H), 2.45-2.41 (m, 2H), 2.33-2.25 (m, 4H), 2.23- |
| | 2.09 (m, 2H), 1.78-1.63 (m, 4H), 1.50 (br d, J = 6.0 Hz, 2H), 1.36 (br |
| | s, 2H), 1.23 (br s, 2H) |
| 624 | (400 MHz, CDCl3) δ 10.51 (br s 1H), 8.96 (br s, 1H), 7.92 (br s, 1H), |
| | 7.49 (br d, J = 8.4 Hz, 1H), 7.24 (br d, J = 7.2 Hz, 2H), 7.01 (br t, J = |
| | 8.0 Hz, 1H), 6.31 (d, J = 7.2 Hz, 1H), 5.16-5.03 (m, 1H), 4.66 (br s, |
| | 1H), 4.44 (dd, J1 = 8.0 Hz, J2 = 2.8 Hz, 1H), 4.00-3.88 (m, 2H), 3.40- |
| | 3.36 (m, 2H), 3.11 (d, J = 11.6 Hz, 1H), 2.96-2.89 (m, 2H), 2.70 (t, |
| | J = 6.0 Hz, 2H), 2.49-2.41 (m, 3H), 2.35-2.30 (m, 1H), 2.23-2.16 |
| | (m, 1H), 2.07-1.99 (m, 2H), 1.88-1.76 (m, 4H), 1.57-1.46 (m, 2H), |
| | 1.31-1.23 (m, 4H), 0.93-0.81 (m, 2H) |
| 625 | (400 MHz, CDCl3) δ = 10.55-10.37 (m, 1H), 8.89-8.73 (m, 1H), |
| | 7.74-7.68 (m, 1H), 7.55-7.49 (m, 1H), 7.45-7.40 (m, 2H), 7.36 (d, |
| | J = 7.6 Hz, 2H), 7.34-7.30 (m, 1H), 7.23 (d, J = 7.2 Hz, 1H), 7.20- |
| | 7.16 (m, 1H), 6.28 (d, J = 7.2 Hz, 1H), 4.02-3.95 (m, 1H), 3.77-3.71 |
| | (m, 1H), 3.62-3.56 (m, 1H), 3.42 (t, J = 5.6 Hz, 2H), 3.09-2.87 (m, |
| | 4H), 2.87-2.73 (m, 2H), 2.70 (t, J = 6.0 Hz, 2H), 2.53 (s, 1H), 2.32- |
| | 2.20 (m, 2H), 2.10-2.02 (m, 2H), 1.89-1.84 (m, 2H), 1.79 (s, 2H), |
| | 1.59-1.44 (m, 2H) |
| 626 | (400 MHz, DMSO-d6). δ 8.16 (s, 1H), 7.50-7.40 (m, 7H), 7.34- |
| | 7.12 (m, 2H), 6.30 (d, J = 7.6 Hz, 1H), 3.92 (t, J = 7.2 Hz, 1H), 3.61- |
| | 3.47 (m, 3H), 3.24 (t, J = 4.8 Hz, 2H), 2.67-2.60 (m, 3H), 2.49-2.45 |
| | (m, 2H), 2.43-2.18 (m, 5H), 1.77-1.69 (m, 4H), 1.51-1.39 (m, 4H). |
| 627 | (400 MHz, DMSO-d6) δ 8.75 (d, J = 1.2 Hz, 1H), 8.44 (s, 1H), 8.19 |
| | (br s, 1H), 7.91 (s, 1H), 7.69 (br d, J = 7.2 Hz, 2H), 7.50 (br t, J = 7.2 |
| | Hz, 2H), 7.45-7.39 (m, 1H), 7.13 (br d, J = 7.2 Hz, 1H), 6.29 (d, J = |
| | 7.2 Hz, 1H), 3.81 (br t, J = 7.2 Hz, 1H), 3.68-3.55 (m, 2H), 3.25-3.22 |
| | (m, 2H), 2.61 (br t, J = 6.0 Hz, 4H), 2.47-2.44 (m, 2H), 2.31-2.05 |
| | (m, 5H), 1.78-1.63 (m, 4H), 1.54-1.32 (m, 4H) |
| 628 | (400 MHz, CDCl3). δ 10.83-10.44 (m, 1H), 9.01-8.84 (m, 1H), |
| | 8.62 (t, J = 5.2 Hz, 1H), 7.70-7.55 (m, 3H), 7.50-7.38 (m, 3H), 7.35 |
| | (dd, J1 = 4.8 Hz, J2 = 1.6 Hz, 1H), 7.23 (dd, J1 = 7.2 Hz, J2 = 3.2 Hz, |
| | 1H), 6.28 (d, J = 7.2 Hz, 1H), 4.14-3.90 (m, 2H), 3.80-3.71 (m, 1H), |
| | 3.40 (br t, J = 5.6 Hz, 2H), 3.20-3.08 (m, 2H), 3.00-2.92 (m, 1H), |
| | 2.79-2.74 (m, 1H), 2.69 (br t, J = 6.0 Hz, 2H), 2.64-2.55 (m, 2H), |
| | 2.37-2.24 (m, 2H), 2.08-1.96 (m, 2H), 1.90-1.79 (m, 4H), 1.76- |
| | 1.67 (m, 1H), 1.61-1.42 (m, 2H) |
| 629 | (400 MHz, DMSO-d6) δ 8.18 (s, 1H), 7.62-7.38 (m, 6H), 7.31- |
| | 7.18 (m, 1H), 7.12 (d, J = 7.6 Hz, 2H), 6.28 (d, J = 7.2 Hz, 1H), 3.82 |
| | (br t, J = 7.6 Hz, 1H), 3.60-3.55 (m, 2H), 3.24 (t, J = 5.4 Hz, 2H), |
| | 2.61 (t, J = 6.0 Hz, 2H), 2.45 (t, J = 7.6 Hz, 3H), 2.38-2.09 (m, 6H), |
| | 1.81-1.62 (m, 4H), 1.58-1.46 (m, 2H), 1.45-1.33 (m, 2H). |
| 630 | (400 MHz, CDCl3) δ 10.82 (s, 1H), 8.78-8.67 (m, 1H), 7.67-7.56 |
| | (m, 3H), 7.46-7.35 (m, 5H), 7.34-7.28 (m, 1H), 7.24 (d, J = 7.6 Hz, |
| | 1H), 6.31 (d, J = 7.2 Hz, 1H), 3.98-3.89 (m, 1H), 3.85 (dd, J1 = 10.4 |
| | Hz, J2 = 3.6 Hz, 1H), 3.60-3.49 (m, 1H), 3.41 (br t, J = 5.2 Hz, 2H), |
| | 3.22-3.10 (m, 2H), 2.93-2.82 (m, 1H), 2.74-2.54 (m, 4H), 2.35- |
| | 2.20 (m, 2H), 2.07-1.97 (m, 1H), 1.96-1.83 (m, 5H), 1.78-1.66 (m, |
| | 1H), 1.39 (t, J = 11.2 Hz, 1H), 1.12-1.01 (m, 1H), 0.77 (d, J = 6.4 Hz, |
| | 3H) |
| 631 | (400 MHz, CDCl3). δ 7.61-7.57 (m, 3H), 7.43-7.28 (m, 6H), 7.22- |
| | 7.10 (m, 1H), 6.21 (s, 1H), 3.85-3.72 (m, 2H), 3.68-3.59 (m, 1H), |
| | 3.41 (t, J = 5.6 Hz, 2H), 3.31-3.11 (m, 1H), 2.74-2.59 (m, 6H), 2.34- |
| | 2.17 (m, 1H), 1.95-1.85 (m, 7H), 1.76-1.69 (m, 1H), 1.33-1.26 |
| | (m, 2H), 0.93 (d, J = 7.6 Hz, 3H). |
| 632 | (400 MHz, CDCl3). δ 10.57 (br s, 1H), 8.94-8.85 (m, 1H), 7.84 (s, |
| | 1H), 7.81-7.75 (m, 3H), 7.53 (dd, J1 = 8.4 Hz, J2 = 1.2 Hz, 1H), 7.44- |
| | 7.36 (m, 2H), 7.25 (d, J = 7.2 Hz, 1H), 6.31 (d, J = 7.2 Hz, 1H), 4.09- |
| | 3.98 (m, 1H), 3.84 (dd, J1 = 9.6 Hz, J2 = 2.8 Hz, 1H), 3.75-3.64 (m, |
| | 1H), 3.39 (br t, J = 4.8 Hz, 2H), 3.15 (br d, J = 11.6 Hz, 1H), 3.09- |
| | 2.95 (m, 1H), 2.91-2.80 (m, 2H), 2.70 (t, J = 6.0 Hz, 2H), 2.57-2.49 |
| | (m, 1H), 2.41-2.25 (m, 2H), 2.15-1.98 (m, 3H), 1.96-1.83 (m, 3H), |
| | 1.82-1.71 (m, 1H), 1.40 (t, J = 11.2 Hz, 1H), 1.15-1.05 (m, 1H), |
| | 0.84 (d, J = 6.4 Hz, 3H) |
| 633 | (400 MHz, CDCl3). δ 7.82 (s, 1H), 7.78-7.75 (m, 3H), 7.52-7.49 |
| | (m, 1H), 7.44-7.38 (m, 2H), 7.01-6.96 (m, 1H), 5.95 (s, 1H), 3.85 (t, |
| | J = 6.0 Hz, 2H), 3.67-3.62 (m, 1H), 3.95 (t, J = 5.6 Hz, 3H), 2.91- |
| | 2.84 (m, 1H), 2.67-2.40 (m, 6H), 2.19-2.13 (m, 1H), 2.01-1.76 (m, |
| | 8H), 1.38-1.26 (m, 1H), 0.91 (d, J = 5.6 Hz, 3H). |
| 634 | (400 MHz, CDCl3) δ 7.84 (s, 1H), 7.82-7.78 (m, 3H), 7.56 (dd, J1 = |
| | 8.4 Hz, J2 = 1.6 Hz, 1H), 7.45-7.40 (m, 2H), 7.23 (d, J = 7.6 Hz, 1H), |
| | 6.28 (d, J = 7.6 Hz, 1H), 4.01-3.92 (m, 1H), 3.91-3.85 (m, 1H), 3.59- |
| | 3.51 (m, 2H), 3.43-3.38 (m, 2H), 2.90-2.83 (m, 1H), 2.80-2.75 |
| | (m, 1H), 2.70 (t, J = 6.2 Hz, 2H), 2.67-2.60 (m, 1H), 2.57-2.45 (m, |
| | 1H), 2.37-2.28 (m, 1H), 2.18-2.11 (m, 1H), 2.02-1.96 (m, 1H), |
| | 1.94 (s, 1H), 1.92-1.89 (m, 1H), 1.88-1.83 (m, 3H), 1.78-1.70 (m, |
| | 1H), 1.39-1.32 (m, 1H), 1.27 (s, 1H), 0.99-0.93 (m, 3H). |
| 635 | (400 MHz, CDCl3). δ 10.50 (br s, 1H), 8.89 (br s, 1H), 7.84 (s, 1H), |
| | 7.79-7.77 (m, 3H), 7.52 (dd, J1 = 8.8 Hz, J2 = 1.6 Hz, 1H), 7.46- |
| | 7.34 (m, 2H), 7.27-7.22 (m, 1H), 6.31 (d, J = 7.6 Hz, 1H), 4.06-4.00 |
| | (m, 1H), 3.91-3.79 (m, 1H), 3.76-3.62 (m, 1H), 3.39 (t, J = 5.2 Hz, |
| | 2H), 3.14 (br d, J = 11.6 Hz, 1H), 3.09-2.98 (m, 1H), 2.92-2.79 (m, |
| | 2H), 2.70 (t, J = 6.0 Hz, 2H), 2.57-2.46 (m, 1H), 2.41-2.24 (m, 2H), |
| | 2.13-1.98 (m, 3H), 1.95-1.84 (m, 3H), 1.82-1.70 (m, 1H), 1.40 (t, J = |
| | 10.8 Hz, 1H), 1.14-1.06 (m, 1H), 0.84 (d, J = 6.4 Hz, 3H) |
| 636 | (400 MHz, DMSO-d6) δ 8.17 (s, 1H), 7.58-7.40 (m, 6H), 7.26- |
| | 7.19 (m, 1H), 7.12 (d, J = 7.6 Hz, 2H), 6.28 (d, J = 7.3 Hz, 1H), 3.82 |
| | (t, J = 7.2 Hz, 1H), 3.63-3.51 (m, 2H), 3.24 (t, J = 5.1 Hz, 2H), 2.61 |
| | (t, J = 6.0 Hz, 2H), 2.47-2.38 (m, 3H), 2.36-2.12 (m, 6H), 1.76- |
| | 1.66 (m, 4H), 1.48 (d, J = 7.0 Hz, 2H), 1.36 (s, 2H) |
| 637 | (400 MHz, CDCl3) δ 7.60 (d, J = 8.8 Hz, 3H), 7.47-7.38 (m, 5H), |
| | 7.35-7.31 (m, 1H), 7.22 (d, J = 7.2 Hz, 1H), 6.28 (d, J = 7.2 Hz, 1H), |
| | 3.96-3.88 (m, 1H), 3.82-3.75 (m, 1H), 3.53-3.47 (m, 1H), 3.45- |
| | 3.39 (m, 2H), 2.92-2.80 (m, 3H), 2.72-2.66 (m, 3H), 2.44-2.33 (m, |
| | 1H), 2.21-2.07 (m, 2H), 1.96-1.79 (m, 6H), 1.42-1.23 (m, 3H), |
| | 0.96 (d, J = 5.6 Hz, 3H). |
| 638 | (400 MHz, CDCl3). δ 10.82 (br s, 1H), 8.75-8.72 (m, 1H), 7.65- |
| | 7.60 (m, 3H), 7.48-7.35 (m, 5H), 7.34-7.28 (m, 1H), 7.24 (d, J = 7.2 |
| | Hz, 1H), 6.31 (d, J = 7.6 Hz, 1H), 4.03-3.81 (m, 2H), 3.60-3.48 (m, |
| | 1H), 3.41 (t, J = 4.4 Hz, 2H), 3.23-3.10 (m, 2H), 2.95-2.84 (m, 1H), |
| | 2.75-2.53 (m, 4H), 2.36-2.20 (m, 2H), 2.07-1.97 (m, 1H), 1.96- |
| | 1.82 (m, 5H), 1.80-1.64 (m, 1H), 1.39 (t, J = 10.8 Hz, 1H), 1.11- |
| | 1.03 (m, 1H), 0.77 (d, J = 6.8 Hz, 3H). |
| 639 | (400 MHz, CDCl3). δ 10.83 (br s, 1H), 8.77 (t, J = 4.8 Hz, 1H), 7.88 |
| | (s, 1H), 7.84-7.75 (m, 3H), 7.57 (dd, J1 = 8.4 Hz, J2 = 2.0 Hz, 1H), |
| | 7.46-7.37 (m, 2H), 7.25 (d, J = 7.2 Hz, 1H), 6.32 (d, J = 7.2 Hz, 1H), |
| | 4.01-3.89 (m, 2H), 3.67-3.57 (m, 1H), 3.40 (t, J = 5.2 Hz, 2H), 3.24- |
| | 3.12 (m, 2H), 2.96-2.88 (m, 1H), 2.72-2.63 (m, 3H), 2.58 (br d, J = |
| | 2.0 Hz, 1H), 2.38-2.23 (m, 2H), 2.00-1.84 (m, 6H), 1.74-1.62 (m, |
| | 1H), 1.39 (t, J = 11.2 Hz, 1H), 1.14-1.00 (m, 1H), 0.71 (d, J = 6.8 Hz, |
| | 3H) |
| 640 | (400 MHz, CDCl3). δ 10.56 (br s, 1H), 8.87 (br d, J = 2.8 Hz, 1H), |
| | 7.63-7.56 (m, 3H), 7.45-7.35 (m, 5H), 7.33-7.28 (m, 1H), 7.24 (d, |
| | J = 7.2 Hz, 1H), 6.30 (d, J = 7.6 Hz, 1H), 4.04-3.94 (m, 1H), 3.74 |
| | (dd, J1 = 9.6 Hz, J2 = 2.4 Hz, 1H), 3.67-3.57 (m, 1H), 3.41 (br t, J = |
| | 4.8 Hz, 2H), 3.12 (br d, J = 12.0 Hz, 1H), 3.07-2.95 (m, 1H), 2.89- |
| | 2.78 (m, 2H), 2.70 (t, J = 6.0 Hz, 2H), 2.54 (br d, J = 1.6 Hz, 1H), 2.37- |
| | 2.24 (m, 2H), 2.12-1.98 (m, 3H), 1.95-1.83 (m, 3H), 1.79-1.69 |
| | (m, 1H), 1.39 (t, J = 10.8 Hz, 1H), 1.18-1.03 (m, 1H), 0.83 (d, J = 6.4 |
| | Hz, 3H) |
| 641 | (400 MHz, CDCl3) δ 7.83 (s, 1H), 7.80-7.74 (m, 3H), 7.50 (d, J = |
| | 8.8 Hz, 1H), 7.44-7.38 (m, 2H), 7.11-6.98 (m, 1H), 6.19-5.98 (m, |
| | 1H), 3.90-3.82 (m, 2H), 3.70-3.61 (m, 1H), 3.41-3.35 (m, 2H), |
| | 2.85-2.73 (m, 1H), 2.67 (t, J = 6.2 Hz, 2H), 2.63-2.48 (m, 3H), 2.47- |
| | 2.35 (m, 1H), 2.17-2.06 (m, 1H), 1.98-1.91 (m, 2H), 1.90-1.83 |
| | (m, 4H), 1.81-1.74 (m, 1H), 1.39-1.24 (m, 3H), 0.93-0.90 (m, 3H). |
| 642 | (400 MHz, CDCl3). δ 7.62-7.57 (m, 3H), 7.45-7.42 (m, 1H), 7.41- |
| | 7.37 (m, 2H), 7.37-7.34 (m, 2H), 7.33-7.28 (m, 1H), 7.19-7.11 (m, |
| | 1H), 6.24-6.17 (m, 1H), 3.85-3.73 (m, 2H), 3.69-3.57 (m, 1H), |
| | 3.41 (t, J = 5.6 Hz, 2H), 2.69 (t, J = 6.0 Hz, 3H), 2.64-2.56 (m, 2H), |
| | 2.39-2.23 (m, 1H), 2.02-1.83 (m, 6H), 1.80-1.57 (m, 2H), 1.35- |
| | 1.22 (m, 4H), 0.93 (d, J = 6.0 Hz, 3H). |
| 643 | (400 MHz, CDCl3). δ 8.79-8.74 (m, 1H), 7.86 (s, 1H), 7.80-7.77 |
| | (m, 3H), 7.56-7.52 (m, 1H), 7.48-7.34 (m, 2H), 6.33 (d, J = 7.2 Hz, |
| | 1H), 3.98-3.95 (m, 2H), 3.72-3.53 (m, 1H), 3.41 (br t, J = 5.2 Hz, |
| | 2H), 3.23-3.13 (m, 1H), 3.11-3.00 (m, 1H), 2.95-2.86 (m, 1H), |
| | 2.75-2.57 (m, 4H), 2.44-2.26 (m, 2H), 2.05-1.84 (m, 6H), 1.78- |
| | 1.66 (m, 1H), 1.52-1.38 (m, 1H), 1.15-1.03 (m, 1H), 0.73 (d, J = 3.6 |
| | Hz, 3H). |
| 644 | (400 MHz, CDCl3). δ 10.56 (br s, 1H), 8.88-8.86 (m, 1H), 7.66- |
| | 7.55 (m, 3H), 7.46-7.35 (m, 5H), 7.25 (d, J = 7.2 Hz, 1H), 7.26-7.22 |
| | (m, 1H), 6.30 (d, J = 7.6 Hz, 1H), 4.02-3.96 (m, 1H), 3.74 (dd, J1 = |
| | 9.6 Hz, J2 = 2.8 Hz, 1H), 3.68-3.57 (m, 1H), 3.41 (br t, J = 4.4 Hz, |
| | 2H), 3.18-3.08 (m, 1H), 3.06-2.96 (m, 1H), 2.91-2.78 (m, 2H), |
| | 2.70 (t, J = 6.0 Hz, 2H), 2.57-2.51 (m, 1H), 2.38-2.21 (m, 2H), 2.13- |
| | 1.98 (m, 3H), 1.95-1.82 (m, 3H), 1.80-1.68 (m, 1H), 1.39 (t, J = |
| | 10.8 Hz, 1H), 1.18-1.04 (m, 1H), 0.83 (d, J = 6.8 Hz, 3H). |
| 645 | (400 MHz, CDCl3). δ 7.83-7.75 (m, 5H), 7.55 (dd, J1 = 8.8 Hz, J2 = |
| | 1.6 Hz, 1H), 7.45-7.38 (m, 2H), 7.20 (d, J = 7.2 Hz, 1H), 6.27 (d, J = |
| | 7.2 Hz, 1H), 3.98-3.86 (m, 3H), 3.60-3.50 (m, 2H), 3.41-3.38 |
| | (m, 2H), 2.88-2.76 (m, 3H), 2.69 (t, J = 6.4 Hz, 2H), 2.65-2.55 (m, |
| | 1H), 2.36-2.33 (m, 1H), 2.12 (s, 1H), 2.04-1.99 (m, 1H), 1.92-1.76 |
| | (m, 6H), 1.38-1.26 (m, 1H), 0.95 (d, J = 5.6 Hz, 3H). |
| 646 | (400 MHz, CDCl3). δ 7.77-7.72 (m, 1H), 7.61-7.59 (m, 3H), 7.47- |
| | 7.36 (m, 5H), 7.33-7.30 (m, 1H), 7.21 (d, J = 7.2 Hz, 1H), 6.27 (d, |
| | J = 7.2 Hz, 1H), 3.90-3.89 (m, 1H), 3.79-3.76 (m, 1H), 3.54-3.48 |
| | (m, 2H), 3.42-3.40 (m, 2H), 2.83-2.78 (m, 3H), 2.69 (t, J = 6.0 Hz, |
| | 2H), 2.64-2.53 (m, 2H), 2.36-2.34 (m, 1H), 2.11-2.02 (m, 2H), |
| | 1.92-1.77 (m, 6H), 1.35-1.32 (m, 1H), 0.94 (d, J = 5.2 Hz, 3H). |

### FIG. 6

FIG. 6, Scheme 12, above illustrates a general synthesis of compounds of Formula VIII.

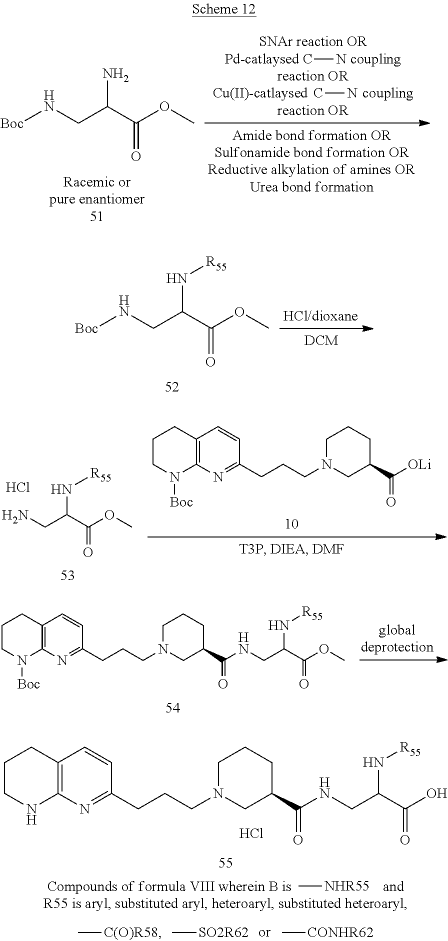

### General Procedure for Preparation of Compound 52 SNAr (Nucleophilic Aromatic Substitution)

To a solution of compound 51 (1.00 eq) in propan-2-ol was added DIEA (2.0) eq) and aryl or heteroaryl halide (1.20 eq). The mixture was stirred at 60° C. for 4 hrs. After the reaction was completed, the reaction mixture was concentrated under reduced pressure to give a residue which was purified by prep-TLC or by flash silica gel chromatography to obtain compound 52.

### Pd-catalyzed C-N Coupling

To a solution of compound 51 in dioxane and toluene was added K₃PO₄ (3.00 eq), TTBP (0.200 eq), the reaction mixture was degassed and purged with N₂ (3×) then Pd₂(dba)₃ or another Pd-catalyst (0.100 eq) was added. The mixture was stirred at 110° C. for 12 hrs under N₂ atmosphere, diluted with H₂O and extracted with EtOAc. The combined organic layers were washed with brine 10.0 mL, dried over Na₂SO₄, filtered and concentrated under reduced pressure to give a residue which was purified by Prep-TLC or by flash silica gel chromatography or by Prep-HPLC to provide compound 52.

### Cu(II)-catalyzed C-N Coupling

To a solution of compound 51 (1.00 eq) and compound aryl/heteroaryl boronic acid/ester (1.20 eq) in DCM was added TEA (2.00 eq), Cu(OAc)₂ or other Cu-catalyst (0.100 eq). The mixture was stirred at 25° C. for 12 hrs, concentrated, diluted by water and extracted with DCM. Then the combined organic layers were dried over Na₂SO₄, filtered and concentrated under reduced pressure to give a residue which was purified by flash silica gel chromatography to provide compound 52.

### Amide Bond Formation

To a solution of compound 51 (1.10 eq) in DMF was added DIEA (4.00 eq), HATU (1.50 eq) and the mixture was stirred for 0.5 hr. Then carboxylic acid (1.00 eq) was added, the mixture was stirred until the reaction was over, diluted with H₂O, extracted with DCM and the combined organic extracts were washed with saturated NaHCO₃ solution and brine, dried over Na₂SO₄, filtered and concentrated under to give a residue which was purified by Prep-TLC or by flash silica gel chromatography or by Prep-HPLC to provide compound 52.

### Sulfonamide Bond Formation

To a solution compound 51 (1.00 eq) in DMF was added sulfonyl halide (0.90 eq) and TEA (2.00 eq) at 0° C. The mixture was stirred at 25° C. for 3 hrs, diluted with H₂O (20.0 mL) and extracted with dichloromethane. The combined organic extracts were washed with brine, dried over Na₂SO₄, filtered and concentrated to give a residue which was purified by prep-TLC or by flash silica gel chromatography to provide compound 52.

### Reductive Alkylation of Amines

A mixture of compound 51 (1.00 eq), aldehyde (1.70 mmol), NaOAc (1.50 eq) and AcOH (0.500 eq) in MeOH was stirred at 25° C. for 1 hr, then NaBH₃CN (2.00 eq) was added and stirred at 25° C. for 2 hrs. Water was added and mixture was extracted with EtOAc, dried over Na₂SO₄ and concentrated under vacuum to give a residue which was purified by prep-TLC or by flash silica gel chromatography to provide compound 52.

### General Procedure for Preparation of Compound 53

To a solution of compound 52 (1.00 eq) in dichloromethane (2.00 mL) was added HCl/dioxane (14.5 eq). The mixture stirred at 25° C. for 2 hrs, concentrated to give a residue which was not purified and used directly for the next reaction. If necessary, the residue was purified by Prep-TLC or by flash silica gel chromatography or by Prep-HPLC to provide compound 53.

### General Procedure for Preparation of Compound 54

To a solution of compound 53 (HCl) and compound 10 (1.00 eq, Li) in dichloromethane (5.00 mL) was added T3P (1.00 eq) and DIEA (4.00 eq) at 0° C. The mixture was stirred at 25° C. for 3 hrs, diluted with saturated NaHCO₃ solution and extracted with dichloromethane. The combined organic extracts were washed with brine, dried over Na₂SO₄, filtered and concentrated to give a residue which was purified by Prep-TLC or by flash silica gel chromatography or by Prep-HPLC to provide compound 54.

### General Procedure for Preparation of a Compound 57

A solution of compound 54 (1.00 eq) in HCl/dioxane (10.00 eq) was stirred at 60° C. for 2 hrs. The reaction mixture was concentrated to give a residue which was purified by flash silica gel chromatography or by Prep-HPLC to obtain compound 55 which is a compound of Formula (VIII) when B is —NHR₅₅.

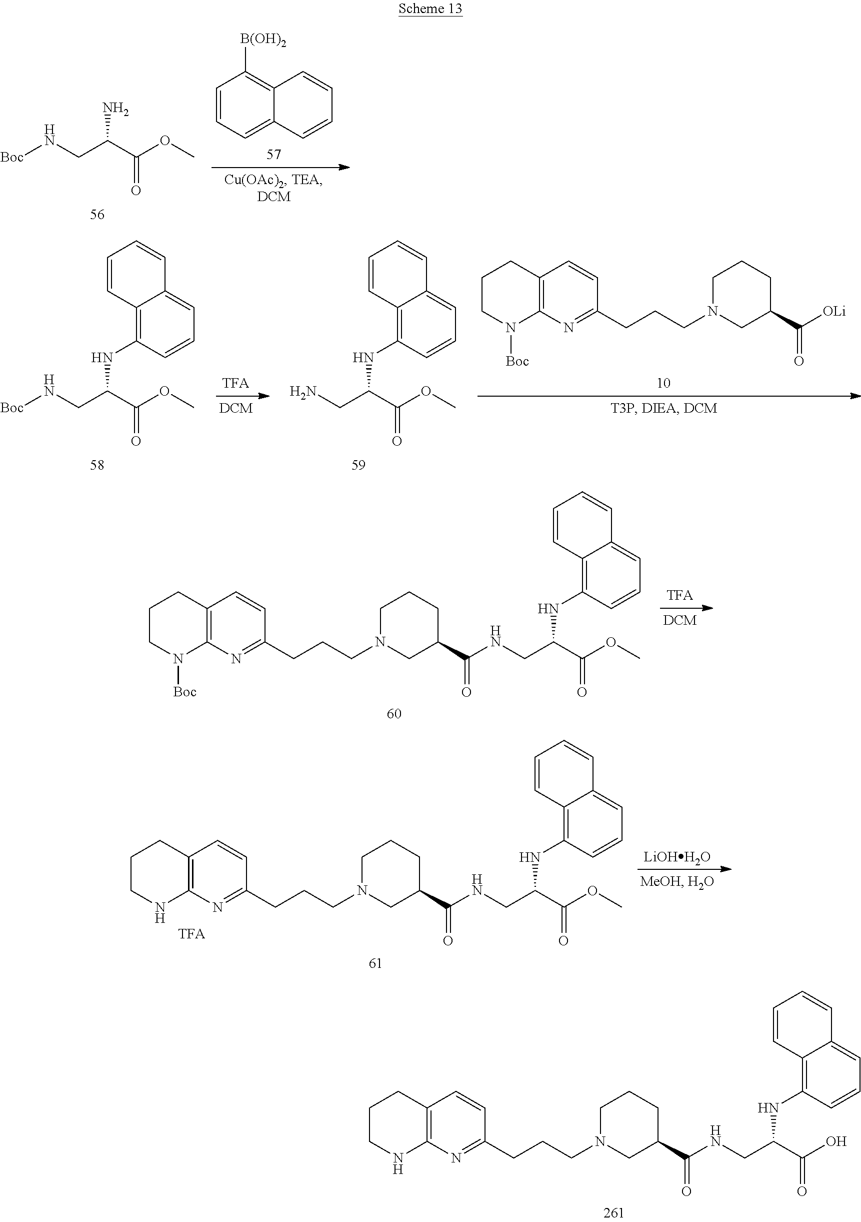

Scheme 13, above, illustrates the synthesis of compound 261 which exemplifies the preparation of compounds of Formula (VIII) shown in FIG. 6., Scheme 12.

### Preparation of compound 58

To a solution of compound 56 (2.00 g, 9.16 mmol, 1.00 eq) and compound 57 (1.89 g, 11.0 mmol, 1.20 eq) in DCM (20.0 mL) was added TEA (1.85 g. 18.3 mmol, 2.55 mL, 2.00 eq), Cu (OAc)₂ (166 mg, 916 umol, 0.100 eq). The mixture was stirred at 25° C. for 12 hrs. TLC (Petroleum ether: Ethyl acetate=3:1, R_{f}(P1)=0.4, I₂) indicated compound 56 was completely consumed and new spots were detected. The reaction mixture was concentrated, diluted with water 30.0 mL, extracted with DCM (30.0 mL*3). The combined organic extracts were dried over Na₂SO₄, filtered and concentrated to give a residue which was purified by flash silica gel chromatography (ISCO®; 8 g SepaFlash® Silica Flash Column, eluent of 0-50% ethyl acetate: petroleum ether gradient @20 mL/min) to provide compound 58 (200 mg, 581 umol, 6.34% yield) as a brown oil. ¹H NMR (400 MHz, CDCl₃) S 7.97-7.95 (m, 1H), 7.81-7.77 (m, ¹H). 7.49-7.45 (m, 2H), 7.34-7.29 (m, 2H), 6.52 (d, J=6.8 Hz, 1H), 5.52 (br s. 1H), 4.95 (br s, 1H), 4.40-4.35 (m, 2H), 3.79 (s, 3H), 3.72-3.62 (m, 1H), 1.47 (s, 9H): LC-MS (M+H)⁺: 345.3.

### Preparation of Compound 59

To a solution of compound 58 (200 mg, 581 umol, 1.00 eq) in DCM (3.00 mL) was added TFA (1.32 g, 11.6 mmol, 860 uL, 20.0 eq). The mixture was stirred at 25° C. for 2 hrs, diluted with DCM (20.0 mL), washed with saturated NaHCO₃ solution (30.0 mL*2) and brine (30.0 mL*1). The organic layer was dried over Na₂SO₄, filtered and concentrated to give a residue containing compound 59 which was used in next step without any purification. LC-MS (M+H)⁺: 245.3.

### Preparation of Compound 60

To a solution of compound 59 (100 mg, 409 umol, 1.00 eq) and compound 10 (192 mg, 450 umol, 1.10 eq, LiOH) in DCM (4.00 mL) was added T3P (521 mg, 819 umol, 487 uL, 50.0% purity, 2.00 eq) and DIEA (212 mg, 1.64 mmol, 285 uL, 4.00 eq). The mixture was stirred at 25° C. for 4 hrs. LC-MS detected the correct mass. The reaction mixture was diluted with H₂O (20.0 mL) and extracted with DCM (20.0 mL*3). The combined organic extracts were washed with saturated NaHCO₃ solution (30.0 mL*2) and brine (30.0 mL*1). The organic layers were dried over Na₂SO₄, filtered and concentrated to give a residue which was purified by Prep-TLC (SiO₂, DCM: MeOH=10:1) to provide compound 60 (38.8% yield) as a yellow solid. LC-MS (M+H)⁺: 630.3; ¹H NMR (400 MHz, CDCl₃) δ 7.95-7.92 (m, 1H), 7.76-7.75 (m, 1H), 7.47-7.42 (m, 2H), 7.30-7.27 (m, 1H), 7.26-7.22 (m, 3H), 6.61 (d, J=7.6 Hz, 1H), 6.48 (d, J=6.8 Hz, 1H), 5.74 (d, J=6.8 Hz, 1H), 4.38-4.33 (m, 1H), 4.07-3.99 (m, 1H), 3.79-3.76 (m, 1H), 3.75 (s, 3H), 3.75-3.68 (m, 3H), 2.96-2.93 (m, 1H), 2.81-2.75 (m, 1H), 2.71 (t, J=6.4 Hz, 2H), 2.61-2.50 (m, 3H), 2.42-2.19 (m, 3H), 1.96-1.85 (m, 4H), 1.82-1.70 (m, 4H), 1.50 (s, 9H).

### Preparation of Compound 61

To a solution of compound 60 (35.0 mg, 55.6 umol, 1.00 eq) in DCM (1.00 mL) was added TFA (127 mg, 1.11 mmol, 82.3 uL, 20.0 eq) at 0° C. The mixture was stirred at 25° C. for 2 hrs.

LC-MS detected the correct mass. The reaction mixture was concentrated to provide a residue containing compound 61 which was used in the next step without any purification. LC-MS (M+H)⁺: 530.5.

### Example 61: (S)-2-(naphthalen-1-ylamino)-3-((R)-1-(3-(5,6,7,8-tetrahydro-1,8-naphthyridin-2-yl)propyl)piperdine-3-carboxamido)propanoic acid (261)

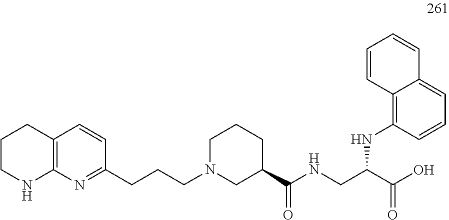 [SMILES: O=C(NC[C@H](Nc1cccc2ccccc12)C(=O)O)[C@@H]1CCCN(CCCc2ccc3c(n2)NCCC3)C1]

To a solution of compound 61 (30.0 mg, 46.6 umol, 1.00 eq, TFA) in MeOH (1.00 mL) was added a solution of LiOH·H₂O (3.91 mg, 93.2 umol, 2.00 eq) in H₂O (0.200 mL) and the mixture was stirred at 25° C. for 5 hrs. LC-MS detected the correct mass. The reaction mixture was concentrated and was purified by Prep-HPLC (column: Waters Xbridge 150*25 mm*5 um; mobile phase: [water (10 mM NH₄HCO₃)−ACN]: B %: 17%-47%, 10 min) and further purified by Prep-SFC (column: DAICEL CHIRALCELOD (250 mm*30 mm, 10 um); mobile phase: [0.1% NH₃H₂O ETOH]: B %: 50%-50%, to provide 261 (14.49 mg, 27.0 umol, 57.9% yield, 96.0% purity) as a yellow solid. LC-MS (M+H)⁺: 516.5: ¹H NMR (400 MHz, CDCl₃) δ 10.6-10.3 (m, 1H), 9.06-8.98 (m, 1H), 8.03-7.96 (m, 1H), 7.82-7.72 (m, 11H), 7.47-7.40 (m, 3H), 7.30-7.27 (m, 1H), 7.27-7.24 (m, 1H), 7.20 (d, J=8.0 Hz, 1H), 6.99 (d, J=8.0 Hz, 1H), 6.31 (d, J=7.2 Hz, 1H), 6.00-5.85 (m, 1H), 4.46-4.39 (m, 1H), 3.94 (d, J=8.8 Hz, 1H), 3.58-3.48 (m, 3H), 3.14-3.08 (m, 1H), 2.98-2.91 (m, 2H), 2.75-2.56 (m, 5H), 2.52-2.31 (m, 3H), 2.17-2.08 (m. 1H), 1.99-1.89 (m, 4H), 1.87-1.79 (m, 1H), 1.68-1.53 (m, 2H).

### Example 62: (S)-2-(cyclohexanecarboxamido)-3-((R)-1-(3-(5,6,7,8-tetrahydro-1,8-naphthyridin-2-yl)propyl)piperidine-3-carboxamido)propanoic acid hydrochloride (262)

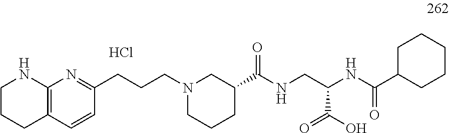 [SMILES: O=C(N[C@@H](CNC(=O)[C@@H]1CCCN(CCCc2ccc3c(n2)NCCC3)C1)C(=O)O)C1CCCCC1 ; Cl]

Compound 262 was prepared using scheme 12 illustrated in FIG. 6. ¹H NMR (400 MHz, DMSO-d₆) δ 14.2 (br s, 1H), 10.7 (br s, 1H), 8.34 (t, J=4.8 Hz, 1H), 8.05 (s, 2H), 7.96 (d, J=8.0 Hz. 1H), 7.63 (d, J=7.6 Hz, 1H), 6.67 (d, J=7.2 Hz, 1H), 3.44-3.43 (M. 4H). 3.26-3.20 (m, 2H), 3.10-3.01 (m, 2H), 2.95-2.81 (m, 3H), 2.77-2.72 (m, 4H), 2.21-2.07 (m, 3H), 1.94-1.76 (m, 5H), 1.72-1.65 (m, 4H), 1.62-1.56 (m, 1H), 1.40-1.16 (m, 6H): LC-MS (M+H)⁺: 500.8.

### Example 63: (S)-2-(1-methylcyclohexane-1-carboxamido)-3-((R)-1-(3-(5,6,7,8-tetrahydro-1,8-naphthyridin-2-yl)propyl)piperidine-3-carboxamido)propanoic acid hydrochloride (263)

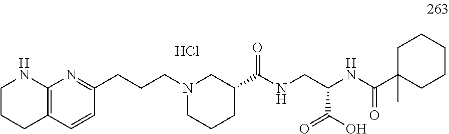 [SMILES: CC1(C(=O)N[C@@H](CNC(=O)[C@@H]2CCCN(CCCc3ccc4c(n3)NCCC4)C2)C(=O)O)CCCCC1 ; Cl]

Compound 263 was prepared using scheme 12 illustrated in FIG. 6. ¹H NMR (400 MHz, DMSO-d₆) δ 14.3 (s, 1H), 10.9 (m, 1H), 8.47 (t, J=5.6 Hz, 1H), 8.20-8.04 (m, 1H), 7.75-7.57 (m, 2H), 6.67 (d, J=7.2 Hz, 1H), 4.32-4.27 (m, 1H), 3.45-3.27 (m, 7H), 3.05 (br s, 2H), 2.93-2.84 (m, 2H), 2.79-2.71 (m, 4H), 2.21-2.06 (m, 2H), 1.99-1.90 (m, 3H), 1.86-1.78 (m, 3H), 1.48-1.07 (m, 10H), 1.03 (s, 3H); LC-MS (M+H)⁺: 514.3.

### Example 64: (S)-2-((1R,2R)-2-(pyrrolidine-1-carbonyl)cyclohexane-1-carboxamido)-3-((R)-1-(3-(5,6,7,8-tetrahydro-1,8-naphthyridin-2-yl)propyl)piperidine-3-carboxamido)propanoic acid (264)

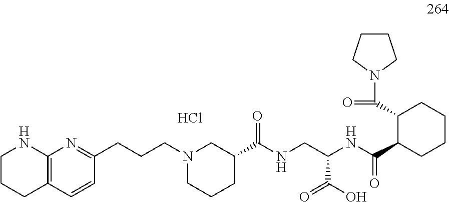 [SMILES: O=C(NC[C@H](NC(=O)[C@@H]1CCCC[C@H]1C(=O)N1CCCC1)C(=O)O)[C@@H]1CCCN(CCCc2ccc3c(n2)NCCC3)C1 ; Cl]

Compound 264 was prepared using scheme 12 illustrated in FIG. 6. ¹H NMR (400 MHz, DMSO-d₆) δ 14.2 (br s, 1H), 10.7 (br s, 1H), 8.29-8.21 (m, 1H), 8.03 (br s, 1H), 7.90 (d, J=8.0 Hz, 1H), 7.62 (d, J=7.2 Hz, 1H), 6.66 (d, J=7.2 Hz, 1H), 4.18 (q, J=6.4 Hz, 1H), 3.55-3.48 (m, 2H), 3.46-3.39 (m, 4H), 3.30 (t, J=6.4 Hz, 2H), 3.23-3.14 (m, 2H), 3.10-3.03 (m, 2H), 2.93-2.83 (m, 2H), 2.78-2.69 (m, 4H), 2.62-2.56 (m, 1H), 2.19-2.08 (m, 2H), 1.91-1.80 (m, 8H), 1.75-1.68 (m, 6H), 1.43-1.38 (m, 1H), 1.28-1.14 (m, 5H); LC-MS (M+H)⁺: 597.5.

### Example 65: (S)-2-((tert-butoxycarbonyl)amino)-3-((R)-1-(3-(5,6,7,8-tetrahydro-1,8-naphthyridin-2-yl)propyl)piperidine-3-carboxamido)propanoic acid (265)

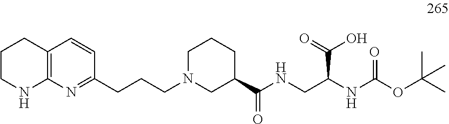 [SMILES: CC(C)(C)OC(=O)N[C@@H](CNC(=O)[C@@H]1CCCN(CCCc2ccc3c(n2)NCCC3)C1)C(=O)O]

Compound 265 was prepared using scheme 12 illustrated in FIG. 6. 1H NMR (400 MHz, CDCl₃) δ 10.4 (br s, 11H), 8.69 (br s, 1H), 7.26 (d, J=7.6 Hz, 1H), 6.30 (d, J=7.2 Hz, 1H), 5.56-5.35 (m, 1H), 4.15 (br s, 1H), 3.94-3.90 (m, 1H), 3.48 (m, J=5.6 Hz, 3H), 2.96-2.91 (m, 2H), 2.77-2.71 (m, 4H), 2.55 (br s, 1H), 2.30-2.29 (m, 3H), 1.91-1.85 (m, 7H), 1.52-1.50 (m, 2H), 1.46 (s, 9H); LC-MS (M+H)⁺: 490.4.

### Example 66: (S)-2-((1R,2R)-2-((3-(methylcarbamoyl)benzyl)carbamoyl)cyclohexane-1-carboxamido)-3-((R)-1-(3-(5,6,7,8-tetrahydro-1,8-naphthyridin-2-yl)propyl)piperidine-3-carboxamido)propanoic acid (266)

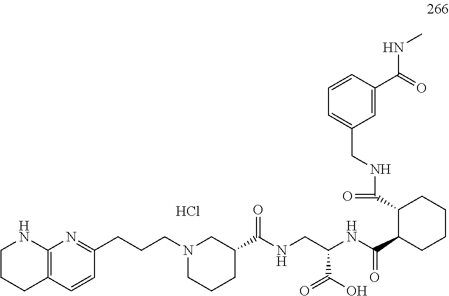 [SMILES: CNC(=O)c1cccc(CNC(=O)[C@@H]2CCCC[C@H]2C(=O)N[C@@H](CNC(=O)[C@@H]2CCCN(CCCc3ccc4c(n3)NCCC4)C2)C(=O)O)c1 ; Cl]

Compound 266 was prepared using scheme 12 illustrated in FIG. 6. ¹H NMR (400 MHz, D₂O) δ 7.60 (t, J=7.6 Hz, 1H), 7.52-7.38 (m, 4H), 6.53-6.50 (m, 1H), 4.44-4.22 (m, 3H), 3.55-3.32 (m, 5H), 3.18-3.07 (m, 2H), 2.97-2.91 (m, 1H), 2.89 (s, 3H), 2.85-2.65 (m, 6H), 2.58-2.48 (m, 2H), 2.12-2.03 (m, 2H), 1.95-1.67 (m, 10H), 1.35-1.29 (m, 5H); LC-MS (M+H)⁺: 690.4.

### Example 67: (S)-2-((R)-1-((1-methylindolin-6-yl)sulfonyl)piperidine-3-carboxamido)-3-((R)-1-(3-(5,6,7,8-tetrahydro-1,8-naphthyridin-2-yl)propyl)piperidine-3-carboxamido)propanoic acid (267)

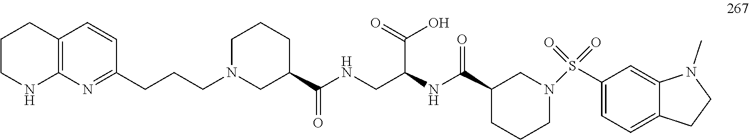 [SMILES: CN1CCc2ccc(S(=O)(=O)N3CCC[C@@H](C(=O)N[C@@H](CNC(=O)[C@@H]4CCCN(CCCc5ccc6c(n5)NCCC6)C4)C(=O)O)C3)cc21]

Compound 267 was prepared using scheme 12 illustrated in FIG. 6. ¹H NMR (400 MHz, DMSO-d₆) δ 13.96-13.95 (m, 1H), 10.52-10.49 (m, 1H), 8.35-8.30 (m, 1H), 8.25-8.23 (m, 1H), 8.02-7.93 (m, 1H), 7.62-7.60 (m, 1H), 7.23 (d, J=7.6 Hz, 1H), 6.87 (d, J=6.8 Hz, 1H), 6.64 (t, J=6.8 Hz, 2H), 4.35-4.31 (m, 1H), 3.59-3.57 (m, 5H), 3.47-3.37 (m, 3H), 2.97-2.90 (m, 3H), 2.59-2.56 (m, 3H), 2.55-2.53 (m, 8H), 2.51-2.49 (m, 5H), 2.33-1.82 (m, 8H), 1.82-1.75 (m, 2H), 1.25-1.23 (m, 2H); LC-MS (M+H)⁺: 696.7.

### Example 68: (S)-2-((1R,2R)-2-((S)-3-methoxypyrrolidine-1-carbonyl)cyclohexane-1-carboxamido)-3-((R)-1-(3-(5,6,7,8-tetrahydro-1,8-naphthyridin-2-yl)propyl)piperidine-3-carboxamido)propanoic acid (268)

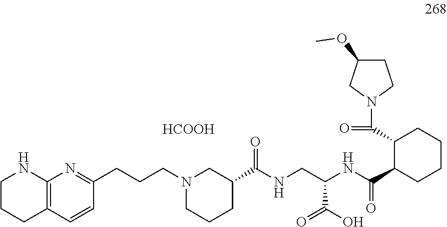 [SMILES: CO[C@H]1CCN(C(=O)[C@@H]2CCCC[C@H]2C(=O)N[C@@H](CNC(=O)[C@@H]2CCCN(CCCc3ccc4c(n3)NCCC4)C2)C(=O)O)C1 ; "HCOOH"]

Compound 268 was prepared using scheme 12 illustrated in FIG. 6. ¹H NMR (400 MHz, CDCl₃) δ 8.20 (s, 1H), 7.99-7.93 (m, 1H), 7.77-7.57 (m, 2H), 7.16 (t, J=7.6 Hz, 1H), 6.34-6.31 (m, 1H), 4.12-3.97 (m, 4H), 3.51-3.33 (m, 5H), 3.31-3.24 (m, 3H), 3.23-3.18 (m, 3H), 3.16-3.02 (m, 2H), 2.93-2.87 (m, 1H), 2.69-2.55 (m, 4H), 2.39-2.23 (m, 4H), 1.99-1.59 (m, 12H), 1.46-1.44 (m, 2H), 1.34-1.17 (m, 4H): LC-MS (M+H)⁺; 627.8.

### Example 69: (S)-2-(((1-methylcyclohexyl)methyl)amino)-3-((R)-1-(3-(5,6,7,8-tetrahydro-1,8-naphthyridin-2-yl)propyl)piperidine-3-carboxamido)propanoic acid (269)

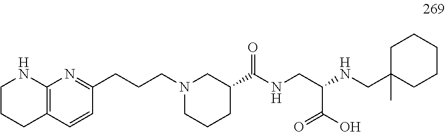 [SMILES: CC1(CN[C@@H](CNC(=O)[C@@H]2CCCN(CCCc3ccc4c(n3)NCCC4)C2)C(=O)O)CCCCC1]

Compound 269 was prepared using scheme 12 illustrated in FIG. 6. ¹H NMR (400 MHz, CDCl₃) δ 10.2 (br s, 11H), 8.92 (br s, 1H), 7.21 (d, J=7.2 Hz, 1H), 6.27 (d, 0.1=7.2 Hz, 1H), 3.94-3.87 (m, 1H), 3.49-3.45 (m, 3H), 3.30-3.22 (m, 2H), 3.09 (dd, J₁=8.4 Hz, J₂=3.2 Hz, 1H), 2.95-2.77 (m, 3H), 2.71 (t, J=6.0 Hz, 2H), 2.67-2.59 (m, 1H), 2.57-2.53 (m, 2H), 2.41-2.26 (m, 3H), 2.01-1.72 (m, 7H), 1.53-1.30 (m, 12H), 0.96 (s, 3H); LC-MS (M+H)⁺: 500.6.

### Example 70: (S)-2-(bicyclo[2.2.2]octane-1-carboxamido)-3-((R)-1-(3-(5,6,7,8-tetrahydro-1,8-naphthyridin-2-yl)propyl)piperidine-3-carboxamido)propanoic acid (270)

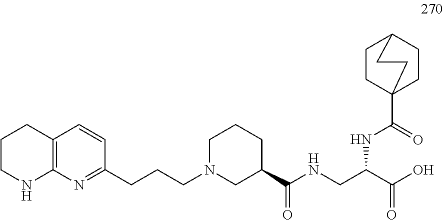 [SMILES: O=C(NC[C@H](NC(=O)C12CCC(CC1)CC2)C(=O)O)[C@@H]1CCCN(CCCc2ccc3c(n2)NCCC3)C1]

Compound 270 was prepared using scheme 12 illustrated in FIG. 6. ¹H NMR (400 MHz, CDCl₃) δ 10.06 (s. 1H). 8.75 (s. 1H). 7.25 (s. 1H), 6.87 (d, J=6.4 Hz, 1H), 6.30 (d, J=7.2 Hz, 1H), 4.46-4.40 (m, 1H), 3.81-3.74 (m, 1H), 3.73-3.51 (m, 2H), 3.48 (t, J=4.8 Hz, 2H), 2.96 (t, J=13.6 Hz, 2H), 2.89-2.80 (m, 1H), 2.75-2.66 (m, 3H), 2.54 (s, 1H), 2.62 (t, J=5.2 Hz, 2H), 2.16 (d, J=9.6 Hz, 1H), 1.98 (d, J=12.4 Hz, 1H), 1.94-1.84 (m, 4H), 1.80-1.75 (m, 6H), 1.62-1.45 (m, 10H): LC-MS (M+H)⁺: 526.4.

### Example 71: (S)-2-(quinazolin-4-ylamino)-3-((R)-1-(3-(5,6,7,8-tetrahydro-1,8-naphthyridin-2-yl)propyl)piperidine-3-carboxamido)propanoic acid (271)

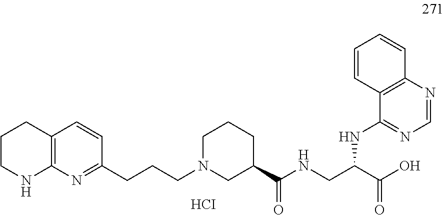

Compound 271 was prepared using scheme 12 illustrated in FIG. 6. ¹H NMR (400 MHz, DMSO-d₆) δ 8.96 (s, 1H), 8.72 (d, J=8.0 Hz, 1H), 8.10 (t, J=7.6 Hz, 1H), 7.94 (d, J=8.4 Hz, 1H), 7.85 (t, J=7.6 Hz, 1H), 7.61 (d, J=7.6 Hz, 1H), 6.65 (d, J=7.2 Hz, 1H), 5.14-5.07 (m, 1H), 3.93-3.88 (m, 1H), 3.43-3.36 (m, 3H), 3.07-3.02 (m, 3H), 2.93-2.81 (m, 3H), 2.75-2.71 (m, 4H), 2.17-1.95 (m, 3H), 1.86-1.74 (m, 5H), 1.51-1.34 (m, 1H); LC-MS (M+H)⁺: 518.3.

### Example 72: (S)-2-(phenylsulfonamido)-3-((R)-1-(3-(5,6,7,8-tetrahydro-1,8-naphthyridin-2-yl)propyl)piperidine-3-carboxamido)propanoic acid (272)

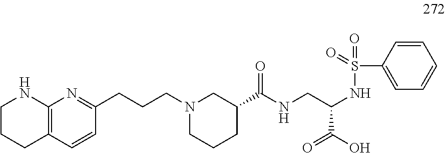 [SMILES: O=C(NC[C@H](NS(=O)(=O)c1ccccc1)C(=O)O)[C@@H]1CCCN(CCCc2ccc3c(n2)NCCC3)C1]

Compound 272 was prepared using scheme 12 illustrated in FIG. 6. ¹H NMR (400 MHz, CDCl₃) δ 10.13 (s, 1H), 8.58 (br s, 1H), 8.04-7.89 (m, 2H), 7.56-7.43 (m, 3H), 7.27-7.24 (m, 1H), 6.30 (d, J=7.2 Hz, 1H), 5.97 (s, 1H), 4.05-3.93 (m, 1H), 3.63 (d, J=7.6 Hz, 1H), 3.48-3.42 (m, 2H), 3.30-3.19 (m, 1H), 2.82-2.63 (m, 6H), 2.53 (s, 1H), 2.44-2.19 (m, 4H), 1.94-1.91 (m, 2H), 1.86-1.80 (m, 2H), 1.70-1.63 (m, 1H), 1.56-1.47 (m, 2H), 1.32-1.25 (m, 1H); LC-MS (M+H)⁺: 530.4.

### Example 73: (S)-2-(quinolin-4-ylamino)-3-((R)-1-(3-(5,6,7,8-tetrahydro-1,8-naphthyridin-2-yl)propyl)piperidine-3-carboxamido)propanoic acid (273)

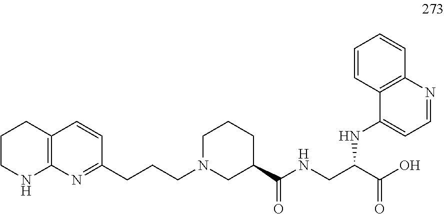 [SMILES: O=C(NC[C@H](Nc1ccnc2ccccc12)C(=O)O)[C@@H]1CCCN(CCCc2ccc3c(n2)NCCC3)C1]

Compound 273 was prepared using scheme 12 illustrated in FIG. 6. ¹H NMR (400 MHz, CDCl₃) δ 10.19 (s, 1H), 9.07 (br s, 1H), 8.58 (d, J=5.6 Hz, 1H), 8.07 (d, J=8.4 Hz, 1H), 7.99 (d, J=8.4 Hz, 1H), 7.64 (t, J=7.6 Hz, 1H), 7.46 (t, J=7.6 Hz, 1H), 7.30 (d, J=7.2 Hz, 1H), 7.16-6.94 (m, 1H), 6.88 (d, J=6.0 Hz, 1H), 6.35 (d, J=7.2 Hz, 1H), 4.36-4.26 (m, 1H), 4.07-3.99 (m, 1H), 3.53 (t, J=5.2 Hz, 2H), 3.32-3.23 (m, 1H), 3.01-2.84 (m, 3H), 2.81-2.67 (m, 4H), 2.62 (s, 1H), 2.39-2.32 (m, 2H), 2.09 (d, J=10.8 Hz, 1H), 1.99-1.76 (m, 7H), 1.63-1.60 (m, 1H): LC-MS (M+H)⁺: 517.

### Example 74: (S)-2-(cyclohexanesulfonamido)-3-((R)-1-(3-(5,6,7,8-tetrahydro-1,8-naphthyridin-2-yl)prop yl)piperidine-3-carboxamido)propanoic acid hydrochloride (274)

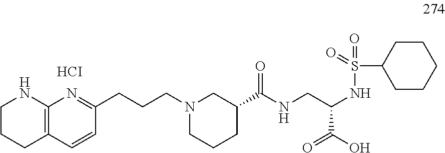

Compound 274 was prepared using scheme 12 illustrated in FIG. 6. ¹H NMR (400 MHz, DMSO-d₆) δ 14.19 (br s, 1H), 10.77 (br s, 1H), 8.41-8.27 (m, 1H), 8.02 (br s, 1H), 7.62 (d, J=7.2 Hz, 1H), 7.47 (d, J=9.6 Hz, 1H), 6.67 (d, J=7.6 Hz, 1H), 3.96-3.90 (m, 1H), 3.46-3.41 (m, 5H), 3.26-3.14 (M, 2H), 3.05 (t, J=7.6 Hz, 2H), 2.92-2.80 (m, 3H), 2.79-2.70 (m, 4H), 2.17-2.07 (m, 3H), 2.01 (d, J=11.2 Hz, 1H), 1.97-1.69 (m, 7H), 1.61 (d, J=12.0 Hz, 1H), 1.51-1.04 (m, 6H): LC-MS (M+H)⁺: 536.3.

### Example 75: (2S)-2-(indan-5-ylsulfonylamino)-3-[[(3R)-1-[3-(5,6,7,8-tetrahydro-1,8-naphthyridin-2-yl)propyl]piperidine-3-carbonyl]amino]propanoic acid (275)

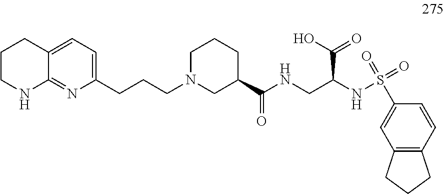 [SMILES: O=C(NC[C@H](NS(=O)(=O)c1ccc2c(c1)CCC2)C(=O)O)[C@@H]1CCCN(CCCc2ccc3c(n2)NCCC3)C1]

Compound 275 was prepared using scheme 12 illustrated in FIG. 6. ¹H NMR (400 MHz, CDCl₃) δ 10.24 (s, 1H), 8.60 (s, 1H), 7.81 (s, 1H), 7.75 (d, J=7.6 Hz, 1H), 7.30 (d, J=8.0 Hz, 1H), 7.25 (s, 1H), 6.29 (d, J=7.2 Hz, 1H), 5.86 (s, 1H), 4.08-4.00 (m, 1H), 3.63 (d, J=8.4 Hz, 1H), 3.49-3.45 (m, 2H), 3.28-3.19 (m, 1H), 2.94 (q, J=7.6 Hz, 5H), 2.85-2.77 (m, 2H), 2.73 (t, J=6.0 Hz, 2H), 2.70-2.63 (m, 1H), 2.53 (s, 1H), 2.35-2.26 (m, 2H), 2.10 (t, J=7.6 Hz, 2H), 1.99-1.90 (m, 4H), 1.84 (br s, 2H), 1.72-1.65 (m, 2H), 1.53-1.47 (m, 2H); LC-MS (M+H)⁺: 570.3.

### Example 76: (2S)-3-[[(3R)-1-[3-(5,6,7,8-tetrahydro-1,8-naphthyridin-2-yl)propyl]piperidine-3-carbonyl]amino]-2-(thieno[3,2-d]pyrimidin-4-ylamino)propanoic acid (276)

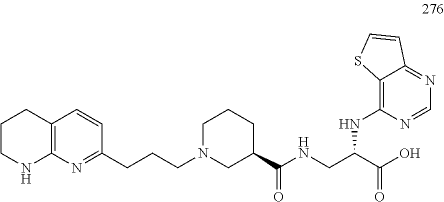 [SMILES: O=C(NC[C@H](Nc1ncnc2ccsc12)C(=O)O)[C@@H]1CCCN(CCCc2ccc3c(n2)NCCC3)C1]

Compound 276 was prepared using scheme 12 illustrated in FIG. 6. ¹HNMR (400 MHz, CDCl₃) δ 10.20 (s, 11H), 8.77 (s, 11H), 8.64 (s, 11H), 7.66 (d, J=5.2 Hz, 1H), 7.38 (d, J=5.2 Hz, 1H), 7.32-7.28 (m, 1H), 6.58-6.67 (M, 1H), 6.33 (d, J=7.2 Hz, 11H), 4.90-4.82 (m, 1H), 4.11-4.03 (m, 1H), 3.81-3.70 (m, 2H), 3.49 (t, J=5.2 Hz, 2H), 2.99-2.77 (m, 5H), 2.73 (t, J=6.0 Hz, 2H), 2.59 (s, 1H), 2.37-2.31 (m, 2H), 1.92 (t, J=5.6 Hz, 4H), 1.64-1.55 (m, 2H), 1.28-21.25 (m, 2H); LC-MS (M+H)⁺: 524.0.

### Example 77: (2S)-2-[(7-methylthieno[3,2-d]pyrimidin-4-yl)amino]-3-[[(3R)-1-[3-(5,6,7,8-tetrahydro-1,8-naphthyridin-2-yl)propyl]piperidine-3-carbonyl]amino]propanoic acid (277)

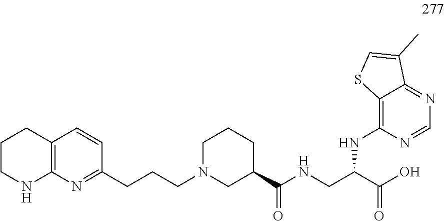 [SMILES: Cc1csc2c(N[C@@H](CNC(=O)[C@@H]3CCCN(CCCc4ccc5c(n4)NCCC5)C3)C(=O)O)ncnc12]

Compound 277 was prepared using scheme 12 illustrated in FIG. 6. ¹H NMR (400 MHz, CDCl₃) δ 10.25 (s. 1H). 8.69 (s. 1H), 7.34-7.28 (m, 1H), 6.53-6.39 (m, 1H), 6.33 (d, J=7.2 Hz, 1H), 4.89-4.73 (m, 1H), 4.23-4.05 (m, 1H), 3.76-3.44 (m, 4H), 3.09-2.98 (m, 2H), 2.92-2.78 (m, 4H), 2.74 (t, J=6.4 Hz, 3H), 2.47-2.43 (m, 3H), 2.38-2.27 (m, 1H), 2.06-1.78 (m, 7H), 1.76-1.65 (m, 1H): LC-MS (M+H)⁺: 538.0.

### Example 78: (2S)-2-[(6,7-difluoroquinazolin-4-yl)amino]-3-[[(3R)-1-[3-(5,6,7,8-tetrahydro-1,8-naphthyridin-2-yl)propyl]piperidine-3-carbonyl]amino]propanoic acid (278)

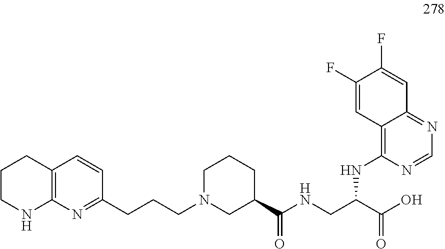 [SMILES: O=C(NC[C@H](Nc1ncnc2cc(F)c(F)cc12)C(=O)O)[C@@H]1CCCN(CCCc2ccc3c(n2)NCCC3)C1]

Compound 278 was prepared using scheme 12 illustrated in FIG. 6. ¹H NMR (400 MHz, DMSO-d₆) δ 8.47-8.41 (m, 2H), 7.77-7.69 (m, 1H), 7.14 (d, J=7.6 Hz, 1H), 6.32 (d, J=7.2 Hz, 1H), 4.83 (t, J=6.8 Hz, 1H), 3.73-3.65 (m, 1H), 3.60-3.48 (m, 4H), 3.24 (t, J=5.6 Hz, 2H), 3.19-3.11 (m, 1H), 3.04-2.95 (m, 1H), 2.87-2.73 (m, 3H), 2.68-2.64 (m, 1H), 2.61 (t, J=6.4 Hz, 3H), 1.92-1.81 (m, 2H), 1.77-1.66 (m, 4H), 1.59-1.42 (m, 2H); LC-MS (M+H)⁺: 554.0.

### Example 79: (S)-3-((R)-1-(3-(5,6,7,8-tetrahydro-1,8-naphthyridin-2-yl)propyl)piperidine-3-carboxamido)-2-(thieno[2,3-d]pyrimidin-4-ylamino)propanoic acid (279)

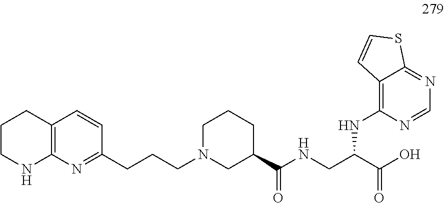 [SMILES: O=C(NC[C@H](Nc1ncnc2sccc12)C(=O)O)[C@@H]1CCCN(CCCc2ccc3c(n2)NCCC3)C1]

Compound 279 was prepared using scheme 12 illustrated in FIG. 6. ¹H NMR (400 MHz, DMSO-d₆) δ 14.33-14.20 (m, 1H), 10.76 (s, 1H), 9.00-8.92 (m, 1H), 8.65-8.62 (m, 1H), 8.51 (s, 1H), 8.01 (s, 1H), 7.92 (d, J=5.76, 1H), 7.72-7.69 (m, 1H), 7.61 (d, J=7.36, 1H), 6.66 (d, J=7.24, 1H), 4.87-4.83 (m, 1H), 3.48-3.41 (m, 5H), 3.04 (s, 2H), 2.91-2.71 (m, 7H), 2.12-2.07 (m, 2H), 1.80-1.64 (m, 4H), 1.41-1.33 (m, 2H); LC-MS (M+H)⁺: 524.3.

### Example 80: (S)-2-((5-methylthieno[2,3-d]pyrimidin-4-yl)amino)-3-((R)-1-(3-(5,6,7,8-tetrahydro-1,8-naphthyridin-2-yl)propyl)piperidine-3-carboxamido)propanoic acid (280)

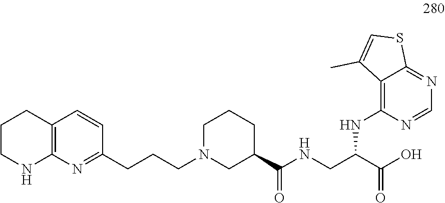 [SMILES: Cc1csc2ncnc(N[C@@H](CNC(=O)[C@@H]3CCCN(CCCc4ccc5c(n4)NCCC5)C3)C(=O)O)c12]

Compound 280 was prepared using scheme 12 illustrated in FIG. 6. ¹H NMR (400 MHz, CDCl₃) δ 10.16-10.12 (m, 111), 8.45 (s, 111), 7.29 (s, 111), 6.92 (d, J=4.4 Hz, 1H), 6.79 (s, 1H), 6.33 (d, J=7.2 Hz, 1H), 4.79-4.78 (m, 1H), 4.11-3.97 (M. 1H), 3.90-3.58 (m, 2H), 3.49 (t, J=5.6 Hz, 2H), 3.08-2.84 (m, 3H), 2.81 (t, J=8.0 Hz, 2H), 2.74 (t, J=6.0 Hz, 2H), 2.69 (s, 3H), 2.61-2.33 (m, 3H), 2.03-1.79 (m, 6H), 1.76-1.51 (m, 2H); LC-MS (M+H)⁺: 538.3.

### Example 81: (2S)-2-(1,3-benzothiazol-2-ylamino)-3-[[(3R)-1-[3-(5,6,7,8-tetrahydro-1,8-naphthyridin-2-yl)propyl]piperidine-3-carbonyl]amino]propanoic acid (281)

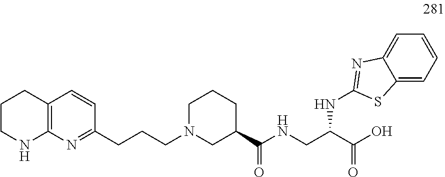 [SMILES: O=C(NC[C@H](Nc1nc2ccccc2s1)C(=O)O)[C@@H]1CCCN(CCCc2ccc3c(n2)NCCC3)C1]

Compound 281 was prepared using scheme 12 illustrated in FIG. 6. ¹H NMR (400 MHz, CDCl₃) δ 7.63 (d, J=7.6 Hz, 1H), 7.35 (d, J=8.0 Hz, 111), 7.23-7.15 (m, 2H), 7.00 (t, J=8.0 Hz, 1H), 6.33 (d, J=7.6 Hz, 11H), 4.33 (s, 1H), 3.46-3.35 (m, 2H), 3.25 (t, J=5.2 Hz, 2H), 2.89-2.79 (m, 1H), 2.62 (t, J=6.0 Hz, 3H), 2.48-2.19 (m, 7H), 1.80-1.68 (m, 4H), 1.65-1.56 (m, 2H), 1.45 (s, 2H); LC-MS (M+H)⁺: 522.6.

### Example 82: (S)-3-((R)-1-(3-(5,6,7,8-tetrahydro-1,8-naphthyridin-2-yl)propyl)piperidine-3-carboxamido)-2-((7-(trifluoromethyl)quinazolin-4-yl)amino)propanoic acid hydrochloride (282)

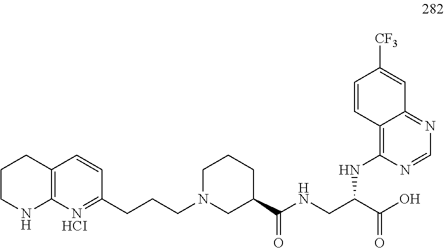

Compound 282 was prepared using scheme 12 illustrated in FIG. 6. ¹H NMR (400 MHz, CDCl₃) δ 14.22 (br s, 1H), 10.79 (br s, 1H), 8.90 (br s, 2H), 8.76-8.57 (m, 1H), 8.31-7.99 (m, 3H), 7.62 (d, J=7.2 Hz, 1H), 6.66 (d, J=7.2 Hz, 1H), 5.05 (br s, 1H), 3.97-3.85 (m, 2H), 3.67-3.54 (m, 2H), 3.46-3.34 (m, 4H), 3.09-2.99 (m, 2H), 2.91-2.79 (m, 2H), 2.77-2.69 (n. 4H), 2.18-2.08 (m, 2H), 1.89-1.69 (m, 5H), 1.48-1.30 (m, 1H); LC-MS (M+H)⁺: 586.0.

### Example 83: (2S)-3-[[(3R)-1-[3-(5,6,7,8-tetrahydro-1,8-naphthyridin-2-yl)propyl]piperidine-3-carbonyl]amino]-2-[[6-(trifluoromethyl)pyrimidin-4-yl]amino]propanoic acid (283)

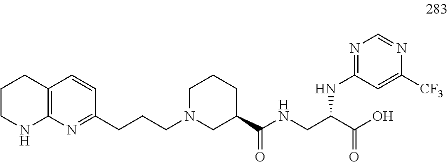 [SMILES: O=C(NC[C@H](Nc1cc(C(F)(F)F)ncn1)C(=O)O)[C@@H]1CCCN(CCCc2ccc3c(n2)NCCC3)C1]

Compound 283 was prepared using scheme 12 illustrated in FIG. 6. ¹H NMR (400 MHz, DMSO-d₆) δ 8.53 (s, 1H), 8.25-8.10 (m, 2H), 7.22 (d, J=7.2 Hz, 1H), 7.08 (s, 1H), 6.36 (d, J=7.2 Hz, 1H), 4.52 (q, J=6.8 Hz, 1H), 3.59-3.49 (m, 2H), 3.40-3.22 (m, 4H), 2.93-2.81 (m, 1H), 2.66-2.54 (m, 4H), 2.44-2.24 (m, 4H), 1.84-1.70 (m, 4H), 1.65-1.54 (m, 2H), 1.47 (s, 2H): LC-MS (M+H)⁺: 536.3.

### Example 84: (S)-2-(3-cyclohexylureido)-3-((R)-1-(3-(5,6,7,8-tetrahydro-1,8-naphthyridin-2-yl)propyl) piperidine-3-carboxamido)propanoic acid (284)

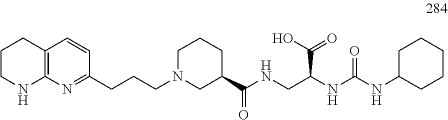 [SMILES: O=C(NC1CCCCC1)N[C@@H](CNC(=O)[C@@H]1CCCN(CCCc2ccc3c(n2)NCCC3)C1)C(=O)O]

Compound 284 was prepared using scheme 12 illustrated in FIG. 6. ¹H NMR (400 MHz, DMSO-d₆) δ 8.12 (br s, 1H), 7.94-7.70 (m, 11H), 7.18 (d, J=7.2 Hz, 1H), 6.34 (d, J=7.2 Hz, 1H), 6.16 (d, J=8.0 Hz, 1H), 5.90 (d, J=7.2 Hz, 1H), 4.01 (q, J=6.8 Hz, 1H), 3.34-3.25 (m, 4H), 3.12-3.03 (m, 2H), 2.85-2.75 (m, 1H), 2.63 (d, J=6.0 Hz, 2H). 2.42-2.12 (m, 6H), 1.78-1.68 (m, 6H), 1.66-1.57 (m, 4H), 1.52-1.42 (m, 3H), 1.27-1.18 (m, 3H), 1.14-1.01 (m, 3H); LC-MS (M+H)⁺: 515.5.

### Example 85: (2S)-2-(bicyclo[22.2] octane-2-carboxamido)-3-((R)-1-(3-(5,6,7,8-tetrahydro-1,8-naphthyridin-2-yl)propyl)piperidine-3-carboxamido)propanoic acid (285)

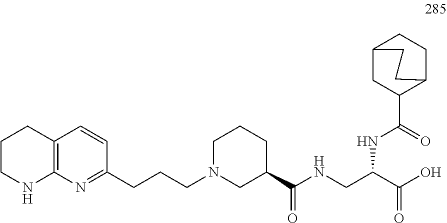 [SMILES: O=C(N[C@@H](CNC(=O)[C@@H]1CCCN(CCCc2ccc3c(n2)NCCC3)C1)C(=O)O)C1CC2CCC1CC2]

Compound 285 was prepared using scheme 12 illustrated in FIG. 6. ¹H NMR (400 MHz, DMSO-d₆) δ 8.09 (s, 1H), 7.70 (dd, J=7.2, J₂=4.8, 1H), 7.46 (m, 1H), 7.16-7.14 (m, 1H), 6.32 (d, J=7.2, 1H), 4.24-4.19 (m, 1H), 3.31-3.22 (m, 5H), 7.72-7.69 (m, 1H), 7.61 (d, J=7.2, 1H), 6.66 (d, J=7.2, 1H), 4.87-4.83 (m, 1H), 3.48-3.41 (m, 5H), 2.84-2.82 (m, 1H), 2.63-2.52 (m, 3H), 2.44-2.25 (m, 7H), 1.89 (s, 1H), 1.78-1.70 (m, 5H), 1.63-1.53 (m, 5H), 1.47-1.40 (m. 7H), 1.25-1.23 (m, 2H); LC-MS (M+H)⁺: 526.1.

The following compounds, set forth in Table 5, were prepared according to the general procedures provided in Scheme 12 or analogous procedures thereto:

TABLE 5

| Compound | |
|---|---|
| No. | ¹H NMR Data |
| 310 | (400 MHz, DMSO-d₆ + D₂O) δ 8.63 (d, J = 8.0 Hz, 1H), 8.13 (s, 1H), 7.54- |
| | 7.43 (m, 1H), 7.24-7.17 (m, 1H), 7.13 (t, J = 8.0 Hz, 2H), 6.36 (d, J = 6.8 |
| | Hz, 1H), 4.33 (t, J = 6.4 Hz, 1H), 3.70-3.63 (m, 2H), 3.31-3.20 (m, 4H), |
| | 2.89 (br s, 1H), 2.66-2.54 (m, 4H), 2.39-2.23(m, 4H), 1.81-1.72 (m, |
| | 4H), 1.66-1.59 (m, 2H), 1.51-1.43 (m, 2H). |
| 311 | (400 MHz, DMSO-d₆) δ 8.18 (s, 1H), 8.08 (br s, 1H), 7.78 (d, J = 8.0 Hz, |
| | 1H), 7.62 (br s, 1H), 7.16 (d, J = 7.2 Hz, 1H), 6.33 (d, J = 7.2 Hz, 1H), |
| | 4.18-4.17 (m, 1H), 3.85-3.82 (m, 3H), 3.45-3.25 (m, 8H), 2.80 (d, J = |
| | 9.2 Hz, 1H), 2.63 (t, J = 6.0 Hz, 2H), 2.43-2.32 (m, 6H), 1.77-1.44 (m, |
| | 12H). |
| 312 | (400 MHz, CDCl₃) δ 10.05 (s, 1H), 8.96 (s, 1H), 8.74-8.70 (m, 2H), 8.02 |
| | (d, J = 6.4 Hz, 1H), 7.78-7.76 (m, 2H), 6.33 (d, J = 7.2 Hz, 1H), 4.75- |
| | 4.70 (m, 1H), 3.95-3.87 (m, 1H), 3.84-3.78 (m, 1H), 3.47 (t, J = 5.6 Hz, |
| | 2H), 3.04-2.99 (m, 2H), 2.96-2.91 (m, 1H), 2.77-2.68 (m, 4H), 2.59 (s, |
| | 1H), 2.32-2.24 (m, 2H), 2.15 (s, 1H), 2.03-1.98 (m, 1H), 1.94-1.88 (m, |
| | 4H), 1.85-1.81 (m, 2H), 1.61-1.54 (m, 2H). |
| 313 | (400 MHz, CDCl₃) δ 10.30 (br s, 1H), 8.75 (br s, 1H), 8.06 (d, J = 6.4 Hz, |
| | 1H), 6.32 (d, J = 7.2 Hz, 1H), 4.61-4.51 (m, 1H), 3.93-3.79 (m, 2H), |
| | 3.49 (t, J = 5.2 Hz, 2H), 3.06-2.88 (m, 2H), 2.80-2.71 (m, 4H), 2.56 (br |
| | s, 1H), 2.39-2.28 (m, 2H), 2.27-2.16 (m, 2H), 1.99-1.86 (m, 6H), 1.78- |
| | 1.71 (m, 1H), 1.58-1.49 (m, 2H), 1.27-1.18 (m, 4H). |
| 348 | (400 MHz, DMSO-d₆) δ 8.42 (d, J = 7.2 Hz, 1H), 8.27-8.15 (m, 1H), 7.98 |
| | (br s, 1H), 7.58 (s, 2H), 7.21 (d, J = 7.2 Hz, 1H), 6.36 (m, 1H), 4.20 (t, J = |
| | 6.0 Hz, 1H), 3.61-3.50 (m, 2H), 3.43-3.34 (m, 1H), 3.28 (t, J = 5.2 Hz, |
| | 2H), 2.95-2.83 (m, 1H), 2.69-2.52 (m, 5H), 2.48-2.30 (m, 4H), 1.87- |
| | 1.70 (m, 4H), 1.69-1.42 (m, 4H). |
| 359 | (400 MHz, DMSO-d₆) δ 8.32 (d, J = 7.4 Hz, 1H), 8.13 (br s, 2H), 7.20 (d, |
| | J = 7.3 Hz, 1H), 6.79 (d, J = 11.8 Hz, 2H), 6.35 (d, J = 7.3 Hz, 1H), 4.97- |
| | 4.87 (m, 1H), 4.29 (q, J = 7.2 Hz, 1H), 4.17 (d, J = 12.8 Hz, 1H), 3.96 (dd, |
| | J₁ = 11.4 Hz, J₂ = 3.5 Hz, 1H), 3.75 (br d, J = 11.4 Hz, 1H), 3.53-3.42 (m, |
| | 5H), 3.30-3.20 (m, 5H), 2.87-2.77 (m, 1H), 2.65-2.62 (m, 2H), 2.41- |
| | 2.36 (m, 2H), 2.29-2.15 (m, 3H), 1.80-1.69 (m, 4H), 1.61 (br d, J = 8.1 |
| | Hz, 2H), 1.48-1.44 (m, 2H). |
| 360 | (400 MHz, DMSO-d₆) δ 8.30 (br s, 1H), 8.15 (d, J = 2.0 Hz, 1H), 7.93 (d, |
| | J = 8.4 Hz, 2H), 7.68 (br d, J = 8.4 Hz, 1H), 7.39 (t, J = 7.6 Hz, 2H), 7.27 |
| | (t, J = 7.2 Hz, 1H), 7.23-7.17 (m, 1H), 7.12 (d, J = 8.4 Hz, 1H), 6.38- |
| | 6.32 (m, 1H), 6.05 (d, J = 5.6 Hz, 1H), 3.93 (d, J = 4.0 Hz, 1H), 3.62-3.51 |
| | (m, 2H), 3.38-3.23 (m, 4H), 2.67-2.61 (m, 2H), 2.59-2.54 (m, 2H), 2.45 |
| | (br s, 2H), 2.41-2.33 (m, 3H), 1.85-1.70 (m, 4H), 1.63-1.43 (m, 4H). |
| 361 | (400 MHz, DMSO-d₆) δ 8.47 (t, J = 5.2 Hz, 1H), 8.08 (dd, J₁ = 14.8 Hz, J₂ = |
| | 1.2 Hz, 2H), 7.66-7.61 (m, 2H), 7.47 (t, J = 7.2 Hz, 2H), 7.41-7.37 (m, |
| | 1H), 7.26-7.19 (m, 2H), 6.38 (d, J = 7.2 Hz, 1H), 6.31 (d, J = 7.6 Hz, 1H), |
| | 4.13-4.09 (m, 1H), 3.58-3.52 (m, 3H), 3.46-3.41 (m, 2H), 3.28 (t, J = |
| | 5.2 Hz, 2H), 3.17-3.11 (m, 1H), 2.97 (br s, 1H), 2.84-2.70 (m, 3H), 2.64 |
| | (t, J = 6.4 Hz, 2H), 2.58-2.53 (m, 1H), 2.02-1.89 (m, 2H), 1.79-1.68 |
| | (m, 5H), 1.57-1.48 (m, 1H). |
| 362 | (400 MHz, DMSO-d₆) δ 8.60 (d, J = 7.6 Hz, 1H), 8.26 (br s, 1H), 8.07 (s, |
| | 1H), 7.48 (d, J = 2.4 Hz, 1H), 7.46 (s, 1H), 7.44-7.38 (m, 1H), 7.22 (d, J = |
| | 7.2 Hz, 1H), 6.36 (d, J = 7.2 Hz, 1H), 4.37-4.32 (m, 1H), 3.64-3.51 |
| | (m, 2H), 3.27 (t, J = 5.6 Hz, 2H), 3.24-3.16 (m, 1H), 2.95-2.86 (m, 1H), |
| | 2.64 (t, J = 6.0 Hz, 2H), 2.60-2.55 (m, 1H), 2.55-2.51 (m, 1H), 2.47- |
| | 2.42 (m, 1H), 2.42-2.23 (m, 3H), 2.21 (br s, 1H), 1.83-1.70 (m, 4H), |
| | 1.63-1.61 (m, 2H), 1.53-1.41 (m, 2H). |
| 363 | (400 MHz, DMSO-d₆) δ 8.74 (d, J = 8.0 Hz, 1H), 8.22 (s, 1H), 7.86 (d, J = |
| | 2.0 Hz, 2H), 7.82-7.81 (m, 1H), 7.17 (d, J = 7.2 Hz, 1H), 6.32 (d, J = 7.2 |
| | Hz, 1H), 4.38 (q, J = 7.6 Hz, 1H), 3.55-3.48 (m, 2H), 3.45-3.38 (m, 2H), |
| | 3.26 (br t, J = 5.6 Hz, 2H), 2.82-2.74 (m, 1H), 2.63 (t, J = 6.0 Hz, 2H), |
| | 2.41-2.29 (m, 3H), 2.27-2.11 (m, 3H), 1.78-1.58 (m, 6H), 1.44 (br t, J = |
| | 8.0 Hz, 2H). |
| 364 | (400 MHz, CDCl₃) δ 10.20 (s, 1H), 8.73 (s, 1H), 7.72 (d, J = 2.0 Hz, 1H), |
| | 7.39 (d, J = 6.4 Hz, 1H), 7.32-7.29 (m, 2H), 7.28 (s, 1H), 6.32 (d, J = 7.2 |
| | Hz, 1H), 4.64-4.60 (m, 1H), 3.94-3.82 (m, 2H), 3.49 (s, 2H), 2.96-2.89 |
| | (m, 2H), 2.80 (d, J = 9.2 Hz, 1H), 2.73 (t, J = 6.0 Hz, 4H), 2.59 (s, 2H), |
| | 2.32 (s, 1H), 1.95-1.91 (m, 4H), 1.85-1.77 (m, 2H), 1.58 (s, 2H). |
| 365 | (400 MHz, DMSO-d₆) δ 8.49 (d, J = 7.8 Hz, 1H), 8.15-8.00 (m, 2H), 7.70 |
| | (t, J = 4.8 Hz, 1H), 7.41 (d, J = 4.6 Hz, 2H), 7.20 (d, J = 7.2 Hz, 1H), 6.35 |
| | (d, J = 7.2 Hz, 1H), 4.34 (q, J = 7.6 Hz, 1H), 3.56-3.49 (m, 2H), 3.38- |
| | 3.31 (m, 2H), 3.28 (br t, J = 5.6 Hz, 2H), 2.80 (br s, 1H), 2.64 (t, J = 6.0 |
| | Hz, 2H), 2.42-2.36 (m, 3H), 2.30-2.18 (m, 3H), 1.80-1.71 (m, 4H), |
| | 1.63-1.61 (m, 2H), 1.52-1.42 (m, 2H). |
| 366 | (400 MHz, CDCl₃) δ 10.07 (s, 1H), 7.29 (s, 1H), 6.97 (s, 1H), 6.33 (d, J = |
| | 7.2 Hz, 1H), 4.46-4.33 (m, 1H), 3.81-3.57 (m, 3H), 3.49 (t, J = 5.6 Hz, |
| | 3H), 3.04-2.99 (m, 1H), 2.87-2.79 (m, 2H), 2.74 (t, J = 6.0 Hz, 3H), 2.34- |
| | 2.25 (m, 1H), 2.23-2.10 (m, 3H), 2.08-1.92 (m, 6H), 1.91-1.79 (m, |
| | 6H), 1.80-1.60 (m, 4H). |
| 367 | (400 MHz, DMSO-d₆) δ 8.64 (d, J = 8.0 Hz, 1H), 8.23 (s, 1H), 8.07 (dd, J₁ = |
| | 7.2 Hz, J₂ = 2.0 Hz, 1H), 7.91-7.84 (m, 1H), 7.83-7.65 (m, 1H), 7.53 |
| | (t, J = 8.8 Hz, 1H), 7.16 (d, J = 7.2 Hz, 1H), 6.32 (d, J = 7.6 Hz, 1H), 4.39 |
| | (dd, J₁ = 14.4 Hz, J₂ = 6.8 Hz, 1H), 3.56-3.38 (m, 3H), 3.26 (t, J = 5.2 Hz, |
| | 2H), 2.82-2.74 (m, 1H), 2.70-2.58 (m, 3H), 2.48-2.42 (m, 1H), 2.41- |
| | 2.33 (m, 2H), 2.31-2.13 (m, 3H), 1.81-1.66 (m, 4H), 1.65-1.57 (m, 2H), |
| | 1.51-1.37 (m, 2H). |
| 368 | (400 MHz, DMSO-d₆) δ 8.68 (d, J = 7.6 Hz, 1H), 8.22 (s, 1H), 8.08 (d, J = |
| | 2 Hz, 1H), 7.82 (dd, J₁ = 8.4 Hz, J₂ = 2.0 Hz, 1H), 7.78-7.73 (m, 1H), |
| | 7.17 (d, J = 7.6 Hz, 1H), 6.32 (d, J = 7.2 Hz, 1H), 4.37 (dd, J₁ = 14.4 Hz, |
| | J₂ = 6.8 Hz, 1H), 3.43-3.39 (m, 4H), 3.26 (t, J = 5.2 Hz, 3H), 2.79-2.72 |
| | (m, 1H), 2.63 (t, J = 6 Hz, 2H), 2.37-2.31 (m, 2H), 2.26-2.11 (m, 3H), |
| | 1.79-1.57 (m, 6H), 1.49-1.38 (m, 2H). |
| 369 | (400 MHz, DMSO-d₆) δ 8.31 (d, J = 7.2 Hz, 1H), 8.25 (s, 1H), 7.73 (d, J = |
| | 8.0 Hz, 2H), 7.59-7.40 (m, 1H), 7.18-7.11 (m, 3H), 6.34-6.28 (m, 1H), |
| | 4.41-4.34 (m, 1H), 3.56-3.49 (m, 3H), 3.44-3.40 (m, 2H), 3.25 (t, J = |
| | 4.8 Hz, 2H), 2.85-2.75 (m, 1H), 2.62 (t, J = 5.6 Hz, 3H), 2.45 (d, J = 8.4 |
| | Hz, 1H), 2.39 (s, 1H), 2.29-2.16 (m, 2H), 2.01-1.92 (m, 1H), 1.80-1.68 |
| | (m, 4H), 1.61 (s, 2H), 1.45 (d, J = 6.4 Hz, 2H). 1.00 (dd, /1 = 13.2 Hz, J₂ = |
| | 5.2 Hz, 2H), 0.75-0.69 (m, 2H). |
| 370 | (400 MHz, CDCl₃) δ 10.16 (m, 1H), 8.81 (br s, 1H), 7.67-7.39 (m, 3H), |
| | 7.33-7.27 (m, 1H), 7.26 (d, J = 4.0 Hz, 1H), 7.17 (d, J = 7.6 Hz, 1H), 6.31 |
| | (d, J = 7.2 Hz, 1H), 4.76-4.57 (m, 1H), 4.02-3.90 (m, 1H), 3.85-3.57 |
| | (m, 2H), 3.48 (d, J = 5.2 Hz, 3H), 3.06-2.81 (m, 3H), 2.79-2.67 (m, 3H), |
| | 2.65-2.51 (m, 1H), 2.32 (br s, 2H), 2.04-1.80 (m, 7H), 1.68-1.49 (m, |
| | 2H), 1.08-0.85 (m, 2H), 0.81-0.67 (m, 2H). |
| 371 | (400 MHz, DMSO-d₆) δ 8.17-8.14 (m, 3H), 7.66 (br s, 1H), 7.31 (t, J = |
| | 7.6 Hz, 2H), 7.16 (t, J = 6.0 Hz, 2H), 6.90 (d, J = 8.0 Hz, 1H), 6.32 (d, J = |
| | 7.2 Hz, 1H), 4.42-4.33 (m, 2H), 3.54-3.48 (m, 1H), 3.45-3.38 (m, 2H), |
| | 3.26 (br t, J = 5.2 Hz, 2H), 2.86 (br s, 1H), 2.63 (br t, J = 6.0 Hz, 2H), 2.45- |
| | 2.22 (m, 7H), 1.83-1.70 (m, 4H), 1.64-1.62 (m, 2H), 1.53-1.35 (m, |
| | 2H), 0.94-0.83 (m, 2H), 0.73-0.60 (m, 2H). |
| 372 | (400 MHz, DMSO-d₆) δ 8.52 (d, J = 8.0 Hz, 1H), 8.23 (s, 1H), 7.90-7.83 |
| | (m, 2H), 7.82-7.58 (m, 1H), 7.57-7.49 (m, 2H), 7.16 (d, J = 7.6 Hz, 1H), |
| | 6.32 (d, J = 7.2 Hz, 1H), 4.40-4.33 (m, 1H), 3.54-3.50 (m, 2H), 3.45- |
| | 3.37 (m, 2H), 3.26 (t, J = 5.6 Hz, 2H), 2.76 (d, J = 9.6 Hz, 1H), 2.62 (t, J = |
| | 6 Hz, 2H), 2.47-2.42 (m, 1H), 2.40-2.31 (m, 2H), 2.30-2.11 (m, 3H), |
| | 1.82-1.65 (m, 4H), 1.65-1.54 (m, 2H), 1.44 (t, J = 8.0 Hz, 2H). |
| 373 | (400 MHz, CDCl₃) δ 10.12 (s, 1H), 8.86 (br s, 1H), 7.91 (s, 1H), 7.77 (d, |
| | J = 7.6 Hz, 1H), 7.69 (br d, J = 5.2 Hz, 1H), 7.44-7.40 (m, 1H), 7.35 (t, J = |
| | 7.6 Hz, 1H), 7.28 (s, 1H), 6.33 (d, J = 7.6 Hz, 1H), 4.72-4.64 (m, 1H), |
| | 3.98-3.89 (m, 1H), 3.81-3.66 (m, 1H), 3.48 (t, J = 5.2 Hz, 2H), 3.09- |
| | 2.82 (m, 3H), 2.79-2.68 (m, 3H), 2.67-2.53 (m, 1H), 2.45-2.10 (m, 3H), |
| | 2.03-1.78 (m, 7H), 1.68-1.48 (m, 2H). |
| 375 | (400 MHz, DMSO-d₆) δ 8.64-8.46 (m, 1H), 8.37 (s, 1H), 8.17 (d, J = 2.4 |
| | Hz, 1H), 7.93 (d, J = 7.2 Hz, 2H), 7.69 (d, J = 8.4 Hz, 1H), 7.39 (t, J = 7.6 |
| | Hz, 2H), 7.27 (t, J = 7.6 Hz, 2H), 7.14 (dd, J₁ = 8.8 Hz, J₂ = 2.8 Hz, 1H), |
| | 6.39 (d, J = 7.2 Hz, 1H), 6.04 (d, J = 7.2 Hz, 1H), 3.90 (q, J = 5.6 Hz, 1H), |
| | 3.69-3.59 (m, 2H), 3.30 (t, J = 4.8 Hz, 2H), 3.19-3.08 (m, 2H), 2.76- |
| | 2.58 (m, 6H), 2.47-2.31 (m, 3H), 1.91-1.86 (m, 1H), 1.80-1.64 (m, 5H), |
| | 1.59-1.50 (m, 2H). |
| 376 | (400 MHz, DMSO-d₆) δ 8.28 (br s, 1H), 8.09-8.04 (m, 2H), 7.66 (d, J = |
| | 7.6 Hz, 2H), 7.47 (t, J = 7.2 Hz, 2H), 7.41-7.34 (m, 1H), 7.30-7.18 (m, |
| | 2H), 6.36 (d, J = 7.2 Hz, 1H), 5.98 (d, J = 8.4 Hz, 1H), 3.97-3.88 (m, 1H), |
| | 3.64-3.53 (m, 4H), 3.29-3.28 (m, 2H), 2.68-2.61 (m, 3H), 2.59-2.54 |
| | (m, 1H), 2.39-2.37 (m, 1H), 2.33-2.23 (m, 4H), 1.83-1.68 (m, 4H), 1.55 |
| | (br s, 3H), 1.45-1.38 (m, 1H) |
| 448 | (400 MHz, CD3CN-d3) δ 10.4-9.94 (m, 1H), 8.61-8.07 (m, 1H), 7.36 (d, |
| | J = 7.2 Hz, 1H), 7.18-7.12 (m, 1H), 7.11-7.06 (m, 1H), 6.85 (d, J = 2.0 |
| | Hz, 1H), 6.39 (d, J = 7.2 Hz, 1H), 6.04-5.81 (m, 1H), 4.05-3.93 (m, 1H), |
| | 3.40-3.28 (m, 5H), 3.11-2.99 (m, 1H), 2.98-2.89 (m, 2H), 2.76 (s, 3H), |
| | 2.74-2.66 (m, 3H), 2.63-2.56 (m, 2H), 2.43-2.34 (m, 4H), 1.89-1.82 |
| | (m, 3H), 1.79-1.72 (m 3H), 1.66-1.40 (m, 4H). |
| 449 | (400 MHz, DMSO-d₆) δ 8.49 (s, 1H), 8.22 (br s, 1H), 8.00 (s, 2H), 7.52- |
| | 7.47 (m, 3H), 7.20-7.14 (m, 2H), 6.34 (d, J = 7.2 Hz, 1H), 4.53 (br s, 1H), |
| | 3.59 (br s, 1H), 3.32-3.27 (m, 4H), 2.82 (br s, 1H), 3.63 (t, J = 6.0 Hz, |
| | 2H), 2.52-2.51 (m, 1H), 2.48-2.43 (m, 1H), 2.38 (br s, 2H), 2.29-2.21 |
| | (m, 3H), 1.77-1.59 (m, 6H), 1.47-1.45 (m, 2H). |
| 450 | (400 MHz, DMSO-d₆) δ 8.05 (s, 1H), 7.42 (dd, J₁ = 8.4 Hz, J₂ = 2.0 Hz, |
| | 1H), 7.34 (d, J = 1.6 Hz, 1H), 7.17 (d, J = 7.2 Hz, 1H), 6.92 (s, 1H), 6.46 |
| | (d, J = 8.4 Hz, 1H), 6.34 (d, J = 7.2 Hz, 1H), 3.41 (s, 2H), 3.39 (s, 2H), |
| | 3.29-3.23 (m, 4H), 3.20-3.14 (m, 2H), 2.92 (t, J = 8.4 Hz, 2H), 2.76 (s, |
| | 3H), 2.63 (t, J = 6.0 Hz, 2H), 2.48-2.41 (m, 4H), 2.39-2.33 (m, 2H), 1.76 |
| | (d, J = 4.4 Hz, 2H), 1.64-1.55 (m, 2H), 1.52-1.43 (m, 2H), 1.24-1.16 |
| | (m, 2H). |
| 451 | (400 MHz, DMSO-d₆) δ 8.63 (s, 2H), 8.18 (br s, 1H), 7.83 (d, J = 7.6 Hz, |
| | 1H), 7.16 (d, J = 7.2 Hz, 1H), 6.32 (d, J = 7.6 Hz, 1H), 4.37 (q, J = 6.4 Hz, |
| | 1H), 3.60-3.53 (m, 1H), 3.47-3.40 (m, 1H), 3.25 (t, J = 5.2 Hz, 2H), 2.83- |
| | 2.80 (m, 1H), 2.64-2.52 (m, 5H), 2.46-2.37 (m, 3H), 2.33-2.26 (m, |
| | 1H), 2.19 (br s, 1H), 1.76-1.73 (m, 4H), 1.61-1.58 (m, 2H), 1.45-1.42 (m, 2H). |
| 526 | (400 MHz, CDCl₃) δ 10.3 (br s, 1H), 8.93 (br s, 1H), 7.29 (s, 1H), 6.81 (d, |
| | J = 6.8 Hz, 1H), 6.65 (s, 1H), 6.32 (d, J = 7.2 Hz, 1H), 4.27-4.21 (m, 1H), |
| | 4.09-4.01 (m, 1H), 3.50 (s, 2H), 3.31-3.23 (m, 1H), 3.02-2.95 (m, 3H), |
| | 2.83-2.69 (m, 5H), 2.56 (s, 1H), 2.46-2.28 (m, 5H), 2.00-1.75 (m, |
| | 12H), 1.67-1.55 (m, 4H), 1.32-1.27 (m, 2H), 0.90-0.88 (m, 6H). |
| 542 | (400 MHz, DMSO-d₆) δ 8.19 (s, 1H), 8.03 (s, 1H), 7.41 (dd, J₁ = 8.4 Hz, |
| | J₂ = 2.0 Hz, 1H), 7.34 (s, 1H), 7.23 (d, J = 7.6 Hz, 1H), 6.92 (d, J = 2.0 Hz, |
| | 1H), 6.45 (d, J = 8.4 Hz, 1H), 6.36 (d, J = 7.2 Hz, 1H), 3.42 (d, J = 7.6 Hz, |
| | 2H), 3.38 (s, 1H), 3.36 (s, 1H), 3.25 (t, J = 5.2 Hz, 2H), 3.09-2.95 (m, |
| | 2H), 2.93-2.85 (m, 3H), 2.75 (s, 3H), 2.64 (t, J = 6.0 Hz, 2H), 2.58-2.52 |
| | (m, 2H), 2.48-2.43 (m, 1H), 2.36-2.19 (m, 4H), 1.78-1.74 (m, 2H), 1.63- |
| | 1.55 (m, 2H), 1.46 (d, J = 8.4 Hz, 2H), 1.25-1.16 (m, 2H). |
| 543 | (400 MHz, CDCl₃) δ 10.14 (br s, 1H), 8.65-8.36 (m, 1H), 7.78 (d, J = 7.6 |
| | Hz, 1H), 7.38 (d, J = 7.6 Hz, 1H), 7.27-7.21 (m, 2H), 6.30 (d, J = 7.2 Hz, |
| | 1H), 5.99 (br s, 1H), 3.97-3.87 (m, 1H), 3.64-3.55 (m, 1H), 3.46 (t, J = |
| | 5.2 Hz, 2H), 3.32 (d, J = 7.2 Hz, 2H), 3.25-3.13 (m, 1H), 2.98-2.85 (m, |
| | 3H), 2.83-2.70 (m, 4H), 2.68-2.62 (m, 1H), 2.55 (br s, 1H), 2.46-2.25 |
| | (m, 3H), 2.17-2.05 (m, 2H), 1.98-1.80 (m, 6H), 1.74-1.62 (m, 1H), 1.58- |
| | 1.45 (m, 2H). |
| 544 | (400 MHz, DMSO-d₆) δ 8.41 (d, J = 7.2 Hz, 1H), 8.25 (s, 1H), 7.85-7.83 |
| | (m, 2H), 7.56-7.46 (m, 3H), 7.17 (d, J = 7.2 Hz, 1H), 6.33 (d, J = 7.2 Hz, |
| | 1H), 4.42-4.32 (m, 1H), 3.59-3.54 (m, 2H), 3.28-3.25 (m, 2H), 2.77- |
| | 2.73 (m, 1H), 2.68-2.63 (m, 3H), 2.40-2.31 (m, 3H), 2.27-2.16 (m, 3H), |
| | 1.79-1.67 (m, 5H), 1.63-1.61 (m, 2H), 1.47-1.42 (m, 2H). |
| 545 | (400 MHz, CDCl₃) δ 10.3 (s, 1H), 8.57 (s, 1H), 7.43 (dd, J₁ = 2.0 Hz, J₂ = |
| | 1.6 Hz, 1H), 7.37 (dd, J₁ = 1.2 Hz, J₂ = 1.6 Hz, 1H), 7.25 (d, J = 7.2 Hz, |
| | 2H), 7.23-7.19 (m, 1H), 6.61 (d, J = 6.4 Hz, 1H), 6.28 (d, J = 7.2 Hz, 1H), |
| | 4.46-4.23 (m, 1H), 3.75-3.71 (m, 4H), 3.47 (t, J = 5.6 Hz, 2H), 2.87- |
| | 2.78 (m, 2H), 2.72 (t, J = 6.2 Hz, 4H), 2.48 (s, 1H), 2.23 (s, 2H), 2.16- |
| | 2.13 (m, 1H), 1.95-1.88 (m, 4H), 1.86-1.82 (m, 2H), 1.76-1.65 (m, 1H), |
| | 1.50 (d, J = 12.8 Hz, 2H). |
| 546 | (400 MHz, DMSO-d₆) δ 8.07 (br s, 1H), 7.68-7.42 (br s, 1H), 7.37-7.26 |
| | (m, 4H), 7.24-7.13 (m, 3H), 6.36-6.30 (m, 1H), 4.13-4.01 (m, 2H), 3.45- |
| | 3.21 (m, 7H), 2.79-2.71 (m, 1H), 2.62 (br t, J = 6.2 Hz, 2H), 2.48-2.43 |
| | (m, 1H), 2.38-2.26 (m, 3H), 2.17 (br d, J = 4.4 Hz, 2H), 1.80-1.70 (m, |
| | 4H), 1.59 (br s, 2H), 1.43 (d, J = 2.8 Hz, 6H). |
| 547 | (400 MHz, DMSO-d₆) δ 14.36 (s, 1H), 10.99 (s, 1H), 8.27 (s, 1H), 8.07 (s, |
| | 1H), 7.62 (d, J = 7.2 Hz, 1H), 7.36 (d, J = 4.4 Hz, 4H), 7.33-7.27 (m, 1H), |
| | 6.75 (d, J = 7.2 Hz, 1H), 6.68 (d, J = 7.6 Hz, 1H), 4.29 (d, J = 4.0 Hz, 1H), |
| | 3.34-3.32 (m, 1H), 3.23-3.12 (m, 2H), 3.07 (s, 2H), 2.88-2.70 (m, 8H), |
| | 2.22-2.09 (m, 2H), 1.99-1.66 (m, 6H), 1.50-1.17 (m, 4H), 1.02-0.94 |
| | (m, 2H). |
| 548 | (400 MHz, CDCl₃) δ 10.17 (s, 1H), 8.81 (s, 1H), 7.74 (s, 1H), 7.66 (d, J = |
| | 7.6 Hz, 1H), 7.48 (d, J = 6.0 Hz, 1H), 7.25-7.22 (m, 1H), 6.31 (d, J = 7.2 |
| | Hz, 1H), 4.66 (q, J = 6.0 Hz, 1H), 4.01-3.95 (m, 1H), 3.67-3.61 (m, 1H), |
| | 3.48 (t, J = 5.2 Hz, 2H), 3.00-2.71 (m, 10H), 2.58 (s, 1H), 2.31-2.20 (m, |
| | 3H), 2.12-1.80 (m, 9H), 1.57-1.50 (m, 2H). |
| 549 | (400 MHz, DMSO-d₆) δ 14.30 (s, 1H), 10.93 (s, 1H), 8.45 (t, J = 5.6 Hz, |
| | 1H), 8.33 (d, J = 7.6 Hz, 1H), 8.06 (s, 1H), 7.62 (d, J = 7.6 Hz, 1H), 7.43 |
| | (d, J = 7.6 Hz, 1H), 7.35 (d, J = 7.2 Hz, 1H), 7.21 (t, J = 7.6 Hz, 1H), 6.67 |
| | (d, J = 7.2 Hz, 1H), 4.55-4.46 (m, 1H), 3.64-3.55 (m, 2H), 3.45-3.34 |
| | (m, 5H), 3.07 (t, J = 9.2 Hz, 4H), 2.92-2.84 (m, 4H), 2.78-2.70 (m, 4H), |
| | 2.17-2.06 (m, 2H), 2.01-1.95 (m, 2H), 1.92-1.73 (m, 5H), 1.48-1.36 |
| | (m, 1H). |
| 550 | (400 MHz, DMSO-d₆) δ 8.53-8.33 (m, 2H), 8.04 (s, 1H), 7.58 (d, J = 7.2 |
| | Hz, 1H), 7.36-7.23 (m, 4H), 7.23-7.16 (m, 1H), 6.65 (d, J = 7.6 Hz, 1H), |
| | 4.32 (dd, J₁ = 12.8 Hz, J₂ = 6.8 Hz, 1H), 3.51-3.46 (m, 4H), 3.44-3.38 |
| | (m, 5H), 3.29-3.21 (m, 2H), 3.04 (t, J = 7.6 Hz, 2H), 2.85 (s, 2H), 2.79- |
| | 2.68 (m, 4H), 2.21-2.03 (m, 2H), 1.80 (s, 4H). |
| 562 | (400 MHz, CDCl₃) δ 9.86 (br s, 1H), 8.78 (s, 1H), 8.28-8.06 (m, 2H), |
| | 7.99 (d, J = 7.2 Hz, 1H), 7.42 (t, J = 7.6 Hz, 1H), 6.32 (d, J = 7.2 Hz, 1H), |
| | 4.99-4.92 (m, 1H), 4.11-3.70 (m, 3H), 3.44 (t, J = 4.8 Hz, 2H), 3.09- |
| | 2.64 (m, 8H), 2.45 (br s, 2H), 2.06-1.85 (m, 6H), 1.71-1.64 (m, 1H), |
| | 1.34-1.25 (m, 1H). |
| 563 | (400 MHz, DMSO-d₆) δ 8.65 (s, 1H), 8.49 (s, 1H), 8.25 (d, J = 4.8 Hz, |
| | 1H), 7.32 (br s, 1H), 7.20-7.16 (m, 1H), 6.36-6.33 (m, 1H), 4.41 (s, 1H), |
| | 3.59 (t, J = 4.8 Hz, 2H), 3.27 (s, 2H), 2.80 (br s, 1H), 2.67-2.61 (m, 3H), |
| | 2.40-2.32 (m, 7H), 1.77-1.76 (m, 4H), 1.62-1.59 (m, 2H), 1.47-1.45 |
| | (m, 2H). |
| 564 | (400 MHz, DMSO-d₆) δ 8.51 (s, 1H), 8.42 (t, J = 6.0 Hz, 1H), 7.98 (d, J = |
| | 4.0 Hz, 2H), 7.70 (br s, 1H), 7.53-7.49 (m, 3H), 7.13-7.10 (m, 2H), 6.74 |
| | (br s, 1H), 6.32 (d, J = 7.2 Hz, 1H), 4.78 (br s, 1H), 3.61-3.54 (m, 2H), |
| | 3.49-3.46 (m, 2H), 3.26 (t, J = 5.2 Hz, 4H), 2.96-2.90 (m, 3H), 2.81- |
| | 2.71 (m, 2H), 2.62 (t, J = 6.0 Hz, 2H), 2.00-1.96 (m, 2H), 1.78-1.74 (m, |
| | 5H), 1.48-1.44 (m, 1H). |
| 567 | (400 MHz, DMSO + D₂O) δ 7.37-7.35 (m, 2H), 7.21-7.18 (m, 3H), 6.90- |
| | 6.88 (m, 1H), 6.35 (dd, J₁ = 7.2 Hz, J₂ = 4.4 Hz, 1H), 4.01 (q, J = 6.0 Hz, |
| | 1H), 3.56-3.48 (m, 1H), 3.45-3.37 (m, 1H), 3.35-3.25 (m, 3H), 2.68- |
| | 2.62 (m, 1H), 2.53-2.51 (m, 3H), 2.50-2.47 (m, 4H), 1.81-1.52 (m, 8H). |
| 588 | (400 MHz, DMSO-d₆) δ 8.25 (s, 1H), 7.22 (d, J = 6.4 Hz, 1H), 7.10-7.02 |
| | (m, 2H), 6.65-6.56 (m, 2H), 6.36 (d, J = 7.2 Hz, 1H), 4.98 (s, 1H), 3.87 |
| | (s, 1H), 3.56-3.50 (m, 1H), 3.47-3.41 (m, 1H), 3.31-3.18 (m, 4H), 3.13- |
| | 2.97 (m, 3H), 2.67-2.69 (m, 4H), 2.45-2.40 (m, 3H), 2.37-2.27 (m, |
| | 4H), 1.80-1.71 (m, 4H), 1.61-1.43 (m, 4H). |
| 589 | (400 MHz, DMSO-d₆ + D₂O) δ 7.15 (d, J = 7.2 Hz, 1H), 7.00 (t, J = 7.2 Hz, |
| | 1H), 6.50-6.44 (m, 3H), 6.33 (d, J = 7.2 Hz, 1H), 3.76 (s, 3H), 3.46-3.43 |
| | (m, 2H), 3.25-3.22 (m, 5H), 2.95-2.78 (m, 5H), 2.67 (t, J = 6.8 Hz, 3H), |
| | 2.60 (t, J = 6.0 Hz, 3H), 1.82 (s, 2H), 1.73 (t, J = 4.8 Hz, 2H), 1.67-1.65 |
| | (m, 2H), 1.52 (s, 2H). |
| 590 | (400 MHz, DMSO-d₆) δ 8.32 (s, 1H), 7.25 (d, J = 6.8 Hz, 1H), 7.11-7.02 |
| | (m, 2H), 6.71-6.58 (m, 2H), 6.37 (d, J = 7.2 Hz, 1H), 4.93 (s, 1H), 3.79 |
| | (s, 1H), 3.74-3.65(m, 2H), 3.31-3.28 (m, 2H), 3.27-3.23 (m, 1H), 3.13- |
| | 3.02 (m, 3H), 2.98-2.91 (m, 1H), 2.70-2.56 (m, 5H), 2.44-2.32 (m, 3H), |
| | 2.28-2.12 (m, 3H), 1.82-1.74 (m, 3H), 1.72-1.58 (m, 3H), 1.55-1.41 |
| | (m, 2H). |
| 591 | (400 MHz, DMSO-d₆) δ 8.35 (br s, 1H), 7.82 (d, J = 4.4 Hz, 1H), 7.27- |
| | 7.21 (m, 1H), 7.11-6.99 (m, 2H), 6.40-6.34 (m, 1H), 5.26-5.17 (m, 1H), |
| | 3.81-3.76 (m, 2H), 3.68-3.63 (m, 2H), 3.48-3.42 (m, 2H), 3.30 (t, J = |
| | 4.8 Hz, 2H), 3.18-3.13 (m, 1H), 2.97-2.91 (m, 3H), 2.65 (t, J = 5.6 Hz, |
| | 3H), 2.45-2.38 (m, 2H), 2.33-2.13 (m, 3H), 1.82-1.74 (m, 3H), 1.71- |
| | 1.57 (m, 3H), 1.56-1.41 (m, 2H). |
| 592 | (400 MHz, DMSO-d₆ + D₂O) δ 7.23 (d, J = 7.2 Hz, 1H), 7.01 (t, J = 7.6 Hz, |
| | 1H), 6.57 (s, 1H), 6.51-6.46 (m, 2H), 6.37 (d, J = 7.2 Hz, 1H), 3.70-3.69 |
| | (m, 1H), 3.62-3.57 (m, 2H), 3.28-3.21 (m, 3H), 3.04-2.86 (m, 4H), 2.64- |
| | 2.54 (m, 6H), 2.45-2.33 (m, 5H), 1.83-1.49 (m, 8H). |
| 593 | (400 MHz, DMSO-d₆) δ 8.29 (br s, 1H), 7.81 (d, J = 4.8 Hz, 1H), 7.23 (d, |
| | J = 6.8 Hz, 1H), 7.10-7.03 (m, 1H), 6.97-6.91 (m, 1H), 6.37 (d, J = 7.2 |
| | Hz, 1H), 5.29 (d, J = 5.2 Hz, 1H), 3.90-3.83 (m, 2H), 3.50 (t, J = 5.2 Hz, |
| | 2H), 3.43-3.41 (m, 2H), 3.30-3.27 (m, 2H), 3.02-2.90 (m, 4H), 2.64 (t, |
| | J = 6.4 Hz, 3H), 2.41 (br s, 2H), 2.35-2.24 (m, 3H), 1.81-1.72 (m, 4H), |
| | 1.64-1.50 (m, 3H), 1.48-1.40 (m, 1H). |

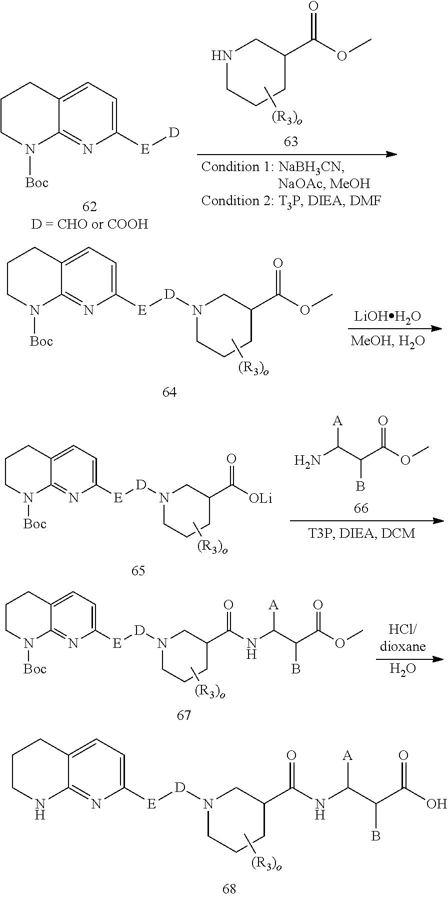

### FIG. 7

FIG. 7, Scheme 14, above, illustrates the general synthesis of compounds of the general formula 68.

### Preparation of Compound 64

Condition 1: To a solution of compound 62 (1.00 eq) and compound 63 (1.1 eq) in MeOH was added NaOAc (2.00 eq) and AcOH (0.200 eq) and the mixture was stirred at 25° C. for 1 hr. Then NaBH₃CN (2.00 eq) was added and the mixture was stirred at 25° C. for 12 hrs. The reaction mixture was concentrated, diluted with H₂O, extracted with ethyl acetate and washed with brine. The organic extracts were dried over Na₂SO₄, filtered and concentrated to give a residue which was purified by Prep-TLC or by flash silica gel chromatography or by Prep-HPLC to provide compound 64.

Condition 2: To a solution of carboxylic acid 62 (1.00 eq) in DMF was added compound 63 (1.05 eq), T3P (1.50 eq) and DIEA (3.00 eq). The mixture was stirred at 25° C. for 16 hrs. The reaction mixture was filtered, and the filtrate purified by Prep-HPLC to provide compound 64.

### Preparation of Compound 65

To a solution of compound 64 (1.00 eq) in MeOH was added a solution of LiOH·H₂O (2.00 eq) in H₂O (0.500 mL), then the mixture was stirred at 25° C. for 2 hrs. After the reaction was completed, the mixture was concentrated to give a residue containing compound 65.

### Preparation of Compound 67

To a solution of compound 65 (1.00 eq) in DMF was added T3P (1.50 eq), amino ester 66 (1.20 eq) and DIEA (3.00 eq), then the mixture was stirred at 25° C. for 2 hrs. After the reaction was completed the mixture was filtered and the residue was purified by Prep-TLC or by flash silica gel chromatography or by Prep-HPLC to provide compound 67.

### Preparation of Compound 68

Compound 67 (1.00 eq) was dissolved in a solution of HCl/dioxane, then the mixture was stirred at 60° C. for 2 hrs. After the reaction was completed, the reaction mixture was concentrated to give a residue. The residue was purified by flash silica gel chromatography or by Prep-HPLC to provide compound 68.

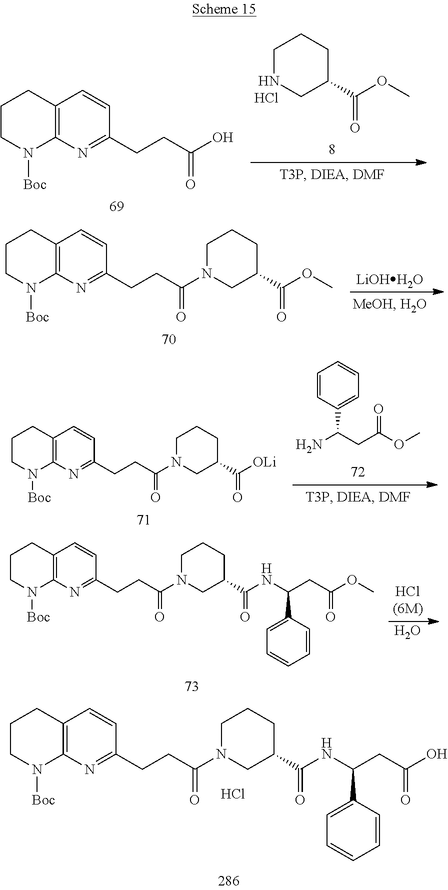

Scheme 15, above, illustrates the synthesis of compound 286 and exemplifies the preparation of compounds of general formula 68 shown in FIG. 7., Scheme 14.

### Preparation of Compound 70

To a solution of compound 69 (200 mg, 653 umol, 1.00 eq) in DMF (2.00 mL) was added compound 8 (123 mg, 685 umol, 1.05 eq, HCl), T3P (623 mg, 979 umol, 582, uL, 50.0% purity, 1.50 eq) and DIEA (253 mg, 1.96 mmol, 341 uL, 3.00 eq). The mixture was stirred at 25° C. for 16 hrs. The reaction mixture was filtered and the filtrate was purified by Prep-HPLC column: Phenomenex Gemini NX-C18 (75*30 mm*3 um): mobile phase: [water (10 mM NH₄HCO₃)−ACN]; B %: 28%-58%. 8 min to provide compound 70 (80.0 mg, 185 umol, 28.4% yield) as white solid. LC-MS (M+H)⁺: 432.3.

### Preparation of Compound 71

To a solution of compound 70 (80.0 mg, 185 umol, 1.00 eq) in MeOH (1.00 mL) was added a solution of LiOH·H₂O (15.6 mg, 371 umol, 2.00 eq) in H₂O (0.500 mL) and the mixture was then stirred at 25° C. for 2 hrs. The mixture was concentrated under reduced pressure to provide compound 71 (78.0 mg, 184 umol, 99.1% yield, Li) as a white solid. LC-MS (M+H)⁺: 418.5.

### Preparation of Compound 73

To a solution of compound 71 (78.0 mg, 184 umol, 1.00 eq, Li) in DMF (1.50 mL) was added T3P (175 mg, 276 umol, 164 uL, 50.0% purity, 1.50 eq) along with compound 72 (47.6 mg, 221 umol, 1.20 eq, HCl) and DIEA (71.3 mg, 551 umol, 96.0 uL, 3.00 eq). The mixture was stirred at 25° C. for 2 hrs, concentrated and the residue purified by Prep-HPLC (column: Waters Xbridge 150*25 mm*5 um: mobile phase: [water (0.05% ammonia hydroxide v/v)−ACN]; B %: 35%-65%, 10 min) to yield compound 73 (40.0 mg, 69.1 umol, 37.6% yield) as an off-white solid. LC-MS (M+H)⁺: 579.5.

### Example 86: (S)-3-phenyl-3-((S)-1-(3-(5,6,7,8-tetrahydro-1,8-naphthyridin-2-yl)propanoyl)piperidine-3-carboxamido)propanoic acid hydrochloride (286)

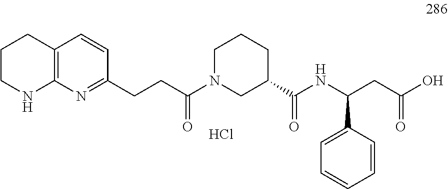 [SMILES: O=C(O)C[C@H](NC(=O)[C@H]1CCCN(C(=O)CCc2ccc3c(n2)NCCC3)C1)c1ccccc1 ; Cl]

Compound 73 (40.0 mg, 69.1 umol, 1.00 eq) was dissolved in a solution of HCl (6.00 M, 2.00 mL, 174 eq) and the mixture was stirred at 60° C. for 2 hrs. The reaction mixture was then concentrated to give a residue which was purified by Prep-HPLC (column: 3_Phenomenex Luna C18 75*30 mm*3 um; mobile phase: [water (0.05% HCl)−ACN]; B %: 14%-34%, 7 min) to yield compound 286 (21.52 mg, 43.0 umol, 62.1% yield, 99.9% purity, HCl) as a white solid. ¹H NMR (400 MHz, DMSO-dh, T=80° C.) S 8.14 (br s, 1H), 8.07-8.04 (m, 1H), 7.57 (d, J=7.6 Hz, 1H), 7.33-7.30 (m, 4H), 7.24-7.22 (m, 1H), 6.61 (d, J=7.6 Hz, 1H), 5.24-5.19 (m, 1H), 3.89-3.87 (m, 1H), 3.44 (t, J=5.6 Hz, 4H), 2.90-2.88 (m, 3H), 2.82-2.81 (m, 2H), 2.76-2.69 (m, 5H), 1.87-1.84 (m, 3H), 1.71-1.67 (m, 2H), 1.39-1.37 (m, 1H); LC-MS (M+H)⁺: 466.5.

### Example 87: (S)-3-phenyl-3-((S)-1-(4-(5,6,7,8-tetrahydro-1,8-naphthyridin-2-yl)butyl)piperidine-3-carboxamido)propanoic acid (294)

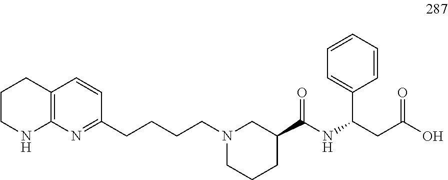 [SMILES: O=C(O)C[C@H](NC(=O)[C@H]1CCCN(CCCCc2ccc3c(n2)NCCC3)C1)c1ccccc1]

Compound 287 was prepared using the method illustrated in scheme 14, FIG. 7. ¹H NMR (400 MHz, DMSO-d₆) δ 14.2 (br s, 1H), 12.3 (br s, 1H), 10.7 (br s, 1H), 8.76 (d, J=6.4 Hz, 1H), 8.02 (br s, 1H), 7.59 (d, J=7.2 Hz, 1H), 7.32-7.31 (m, 4H), 7.26-7.23 (m, 1H), 6.62 (d, J=7.2 Hz, 1H), 5.16-5.12 (m, 1H), 3.41-3.40 (m, 3H), 3.03 (br s, 2H), 2.87-2.81 (m, 3H), 2.73-2.66 (m, 6H), 2.82-1.68 (m, 10H), 1.44 (br s, 1H): LC-MS (M+H)⁺: 465.5.

### Example 88: (S)-3-phenyl-3-((R)-1-(4-(5,6,7,8-tetrahydro-1,8-naphthyridin-2-yl)butyl)piperidine-3-carboxamido)propanoic acid hydrochloride (288)

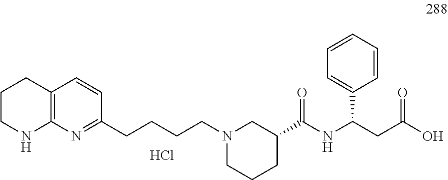 [SMILES: O=C(O)C[C@H](NC(=O)[C@@H]1CCCN(CCCCc2ccc3c(n2)NCCC3)C1)c1ccccc1 ; Cl]

Compound 288 was prepared using the method illustrated in scheme 14, FIG. 7. ¹H NMR (400 MHz, DMSO-d₆) δ 14.09 (s, 1H), 10.62 (brs, 1H), 8.76 (d, J=8.0 Hz, 1H), 8.01 (s, 1H), 7.61 (d, J=7.2 Hz, 1H), 7.33-7.29 (m, 4H), 7.24-7.20 (m, 1H), 6.64 (d, J=7.2 Hz, 1H), 5.18-5.11 (m, 1H), 3.42 (s, 3H), 3.05 (brs, 2H), 2.94-2.88 (m, 2H), 2.78-2.62 (m, 7H), 2.52-2.51 (m, 1H), 1.93-1.86 (m, 2H), 1.83-1.77 (m, 3H), 1.76-1.67 (m, 4H), 1.34-1.30 (m, 1H); LC-MS (M+H)⁺: 465.3.

### Example 89: (S)-3-phenyl-3-((R)-1-(3-(5,6,7,8-tetrahydro-1,8-naphthyridin-2-yl)propanoyl)piperidine-3-carboxamido)propanoic acid hydrochloride (289)

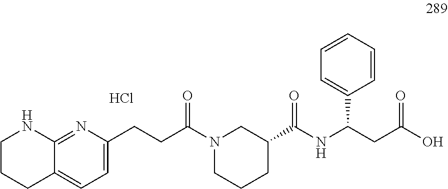 [SMILES: O=C(O)C[C@H](NC(=O)[C@@H]1CCCN(C(=O)CCc2ccc3c(n2)NCCC3)C1)c1ccccc1 ; Cl]

Compound 289 was prepared using the method illustrated in scheme 14, FIG. 7. ¹H NMR (400 MHz, DMSO-d₆) δ 13.99-13.79 (m, 1H), 8.50 (dd, J₁=28.0 Hz, J₂=8.4 Hz, 1H), 8.00 (d, J=16.8 Hz, 1H), 7.60-7.57 (m, 1H), 7.31-7.28 (m, 4H), 7.25-7.20 (m, 1H), 6.66-6.22 (m, 1H), 5.16 (q, J=7.6 Hz, 1H), 4.34-4.31 (m, 1H), 4.09 (d, J=12.0 Hz, 1H), 3.82-3.75 (m, 2H), 3.16-3.08 (m, 1H), 2.96-2.78 (m, 4H), 2.73-2.62 (m, 6H), 1.86-1.42 (m, 6H); LC-MS (M+H)⁺: 465.3.

### Example 90: (S)-3-phenyl-3-((R)-1-(2-(5,6,7,8-tetrahydro-1,8-naphthyridin-2-yl)ethyl)piperidine-3-carboxamido)propanoic acid (290)

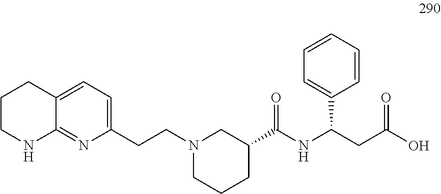 [SMILES: O=C(O)C[C@H](NC(=O)[C@@H]1CCCN(CCc2ccc3c(n2)NCCC3)C1)c1ccccc1]

Compound 290 was prepared using the method illustrated in scheme 14, FIG. 7. ¹H NMR (400 MHz, CDCl₃) δ 9.94 (br s, 2H), 7.38 (d, J=7.2 Hz, 2H), 7.29 (s, 1H), 7.25 (s, 1H), 7.21-7.13 (m, 2H), 6.24 (d, J=7.2 Hz, 1H), 5.39-5.34 (m, 1H), 3.43-3.34 (m, 2H), 3.07 (br s, 1H), 2.94-2.87 (m, 1H), 2.85-2.72 (m, 4H), 2.68 (t, J=6.4 Hz, 2H), 2.65-2.39 (M. 4H), 2.15-1.49 (m, 2H), 1.89-1.83 (m, 2H), 1.78-1.71 (m, 1H), 1.59-1.50 (m, 2H): LC-MS (M+H)⁺: 437.4.

### Example 91: (3S)-3-(5,5-difluoro-1-(3-(5,6,7,8-tetrahydro-1,8-naphthyridin-2-yl)propyl)piperidine-3-carboxamido)-3-phenylpropanoic acid (291)

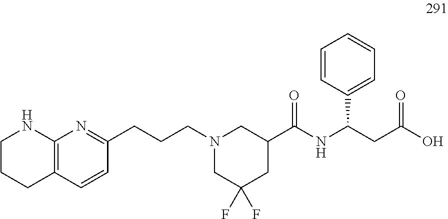 [SMILES: O=C(O)C[C@H](NC(=O)C1CN(CCCc2ccc3c(n2)NCCC3)CC(F)(F)C1)c1ccccc1]

Compound 291 was prepared using the method illustrated in scheme 14, FIG. 7 and the racemate resolved using conventional procedures.

291-A: ¹H NMR (400 MHz, CDCl₃) δ 11.3-10.6 (m, 1H), 9.13 (s, 1H), 7.42 (d, J=7.6 Hz, 2H), 7.35-7.28 (M, 2H), 7.25-7.18 (m, 2H), 6.27 (d, J=7.2 Hz, 1H), 5.36-5.19 (m, 1H), 3.51-3.33 (m, 3H), 2.99-2.86 (m, 1H), 2.85-2.76 (m, 4H), 2.74-2.64 (m, 3H), 2.60-2.43 (m, 3H), 2.26-2.04 (m, 4H), 1.93-1.84 (m, 2H), 1.75-1.63 (m, 1H); LC-MS (M+H)⁺: 487.4.

291-B: ¹H NMR (400 MHz, CDCl₃) δ 10.7 (s, 1H), 9.43 (d, J=7.2 Hz, 1H), 7.41 (d, J=7.6 Hz, 2H), 7.27-7.20 (m, 3H), 7.15 (t, J=7.2 Hz, 1H), 6.26 (d, J=7.2 Hz, 1H), 5.40-5.26 (m, 1H), 3.40 (t, J=5.6 Hz, 2H), 3.03-2.97 (m, 1H), 2.95-2.87 (m, 2H), 2.86-2.82 (m, 3H), 2.68 (t, 0.1=6.0 Hz, 2H), 2.63-2.54 (m, 3H). 2.49-2.42 (m, 2H), 2.15-1.94 (m, 3H), 1.92-1.81 (m, 3H); LC-MS (M+H)⁺: 487.3.

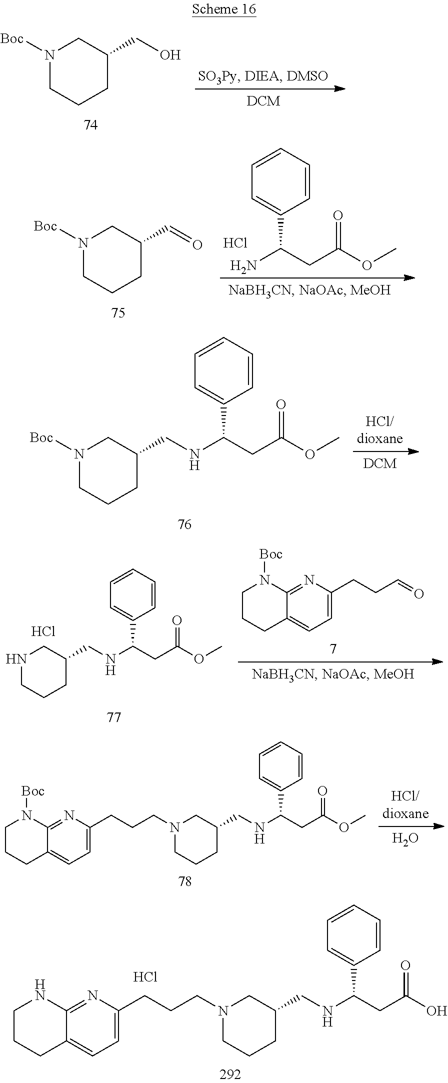

Scheme 16 illustrates a synthesis of compound 292.

### Preparation of Compound 75

To a solution of compound 74 (1.00 g, 4.64 mmol, 1.00 eq) in DCM (10.0 mL) was added DMSO (1.09 g, 13.9 mmol, 1.09 mL, 3.00 eq), DIEA (1.80 g, 13.9 mmol, 2.43 mL, 3.00 eq) and SO₃·Py (2.22 g, 13.9 mmol, 3.00 eq). The mixture was stirred at 25° C. for 2 hrs. The reaction mixture was washed with saturated citric acid solution (20.0 mL*3), dried over Na₂SO₄, filtered and concentrated under reduced pressure to give a residue. The residue was purified by flash silica gel chromatography to obtain compound 75 (1.50 g, crude, 72% yield) as a yellow oil. ¹H NMR (400 MHz, DMSO-d₆) δ 9.59 (s, 1H), 3.85-3.04 (m, 4H), 2.49-2.42 (m, 1H), 1.90-1.82 (m, 1H), 1.70-1.46 (m, 2H), 1.38 (s, 9H), 1.30-1.25 (m, 1H).

### Preparation of Compound 76

To a solution of compound 75 (1.20 g, 5.63 mmol, 1.00 eq) and methyl (S)-3-amino-3-phenylpropanoate (1.46 g, 6.75 mmol, 1.20 eq, HCl) in MeOH (15.0 mL) was added AcONa (600 mg, 7.31 mmol, 1.30 eq) and NaBH₃CN (707 mg, 11.2 mmol, 2.00 eq). The mixture was stirred at 25° C. for 2 hrs. The reaction mixture was quenched by addition water (10.0 mL) at 0° C., and then extracted with ethyl acetate (20.0 mL*3). The combined organic layers were washed with brine (30.0 mL), dried over Na₂SO₄, filtered and concentrated under reduced pressure to give a residue. The residue was purified by column chromatography (SiO₂, Petroleum ether: Ethyl acetate=1:0 to 0:1. Petroleum ether: Ethyl acetate=1:1, R_{f}═0.40). Compound 76 (0.380 g, 1.01 mmol) was obtained as a white solid. ¹H NMR (400 MHz, DMSO-d₆) δ 7.35-7.26 (m, 4H), 7.24-7.20 (m, 1H), 3.9-3.75 (m, 2H), 3.34-3.62 (m, 1H), 3.63 (s, 3H), 3.30-3.14 (m, 1H), 2.80-2.63 (m, 2H), 2.58-2.52 (m, 1H), 2.20-2.07 (m, 2H), 1.75-1.62 (m, 1H), 1.55-1.46 (m, 1H), 1.38 (s, 2H), 1.37 (s, 9H), 1.28-1.21 (m, 1H), 1.10-0.94 (m, 1H): LC-MS (M+H)⁺: 377.3.

### Preparation of Compound 77

To a solution of compound 76 (150 mg, 398 umol, 1.00 eq) in DCM (2.00 mL) was added HC/dioxane (4.00 M, 99.6 uL, 1.00 eq). The mixture was stirred at 25° C. for 2 hrs. The reaction mixture was concentrated under reduced pressure to give compound 77 (124 mg, crude, 92.0% yield HCl) as a white solid. LC-MS (M+H)⁺: 277.3.

### Preparation of Compound 78

To a solution of compound 77 (100 mg, 320 umol, 1.06 eq, HCl) in MeOH (2.00 mL) was added AcONa (32.2 mg, 392 umol, 1.30 eq), NaBH₃CN (19.0 mg, 301 umol, 1.00 eq) and compound 7 (87.6 mg, 301 umol, 1.00 eg). The reaction mixture was quenched by addition water (2.00 mL) at 0° C., and then extracted with ethyl acetate (5.00 mL*3). The combined organic layers were washed with brine (5.00 mL), dried over Na₂SO₄, filtered and concentrated under reduced pressure to give a residue. The residue was purified by prep-HPLC (basic condition, column: Phenomenex Gemini-NX C18 75*30 mm*3 um; mobile phase: [water (10 mM NH₄HCO₃)−ACN]: B %: 40%-70%, 8 min) to yield compound 78 (50.0 mg, 90.8 umol, 30.1% yield) as a colorless oil. LC-MS (M+H)⁺: 551.6.

### Example 92: (S)-3-phenyl-3-(S)-1-(3-(5,6,7,8-tetrahydro-1,8-naphthyridin-2-yl)propyl)piperidin-3-yl)methyl)amino)propanoic acid hydrochloride (292)

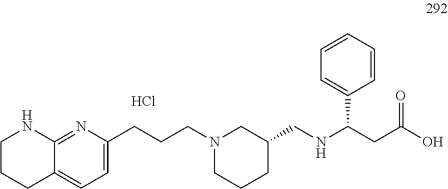 [SMILES: O=C(O)C[C@H](NC[C@@H]1CCCN(CCCc2ccc3c(n2)NCCC3)C1)c1ccccc1 ; Cl]

To a solution of compound 78 (40.0 mg, 72.6 umol, 1.00 eq) in H₂O (1.00 mL) was added HCl/dioxane (4.00 M, 1.45 mL, 80.0 eq). The mixture was stirred at 60° C. for 4 hrs. The reaction mixture was concentrated under reduced pressure to give a residue which was purified by pre-HPLC to provide compound 292 (29.36 mg, 60.0 umol, 82.6% yield, 96.7% purity, HCl) as a yellow solid. ¹H NMR (400 MHz, DMSO-d₆) δ 14.3 (s, 1H), 10.9 (s, 1H), 10.1 (s, 1H), 9.75 (s, 1H), 8.09 (s, 1H), 7.72-7.58 (m, 3H), 7.46-7.35 (m, 3H), 6.70 (d, J=7.2 Hz, 1H), 4.52 (s, 1H), 3.69 (d, J=10.8 Hz, 2H), 3.42-3.25 (m, 2H), 3.17-2.90 (m, 4H), 2.87-2.54 (m, 7H), 2.45-2.29 (m, 2H), 2.22-2.09 (m, 2H), 2.01-1.63 (m, 5H), 1.10-0.95 (m, 1H): LC-MS (M+H)⁺: 437.2.

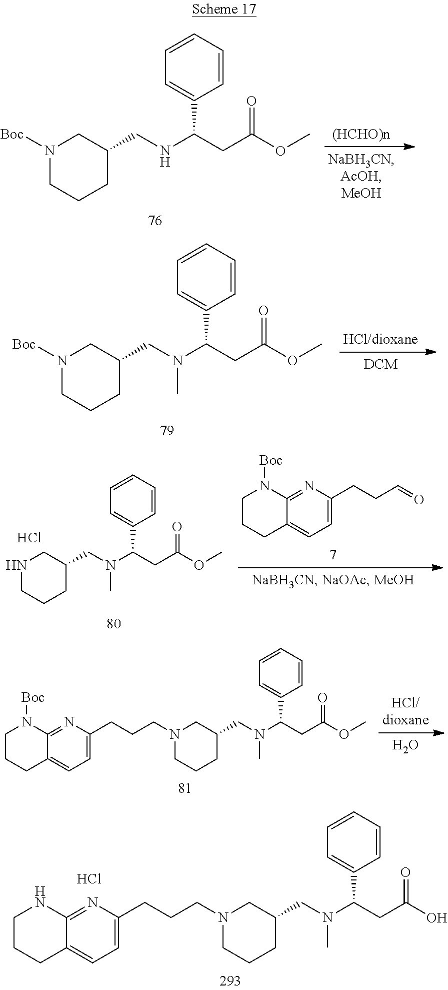

Scheme 17 illustrates the synthesis of compound 293.

### Preparation of Compound 79

To a solution of compound 76 (0.250 g, 664 umol, 1.00 eq) in MeOH (4.00 mL) was added HCHO (23.9 mg, 797 umol, 21.9 uL, 1.20 eq), NaBH₃CN (83.5 mg, 1.33 mmol, and AcOH (399 ug, 6.64 umol, 0.380 uL, 0.0100 eq). The reaction mixture was quenched by addition of water (4.00 mL) at 0° C., and then extracted with ethyl acetate (5.00 mL*3). The combined organic layers were washed with brine (5.00 mL), dried over Na₂SO₄, filtered and concentrated under reduced pressure to give a residue. The residue was purified by prep-TLC (SiO₂, Petroleum ether. Ethyl acetate=1:1, R_{f}=0.60) to provide compound 79 (160 mg, crude) as a colorless oil. ¹H NMR (400 MHz, DMSO-d₆) δ 7.36-7.22 (m, 5H), 4.10-4.00 (m, 1H), 3.88 (d, J=12.2 Hz, 1H), 3.76 (d, J=12.8 Hz, 1H), 3.56 (s, 3H), 2.99 (q, J=8.4 Hz, 1H), 2.78-2.62 (m, 2H), 2.40-2.22 (m, 1H), 2.09 (d, J=7.6 Hz, 2H), 2.00 (s, 3H), 1.71-1.60 (m, 1H), 1.57-1.45 (m, 2H), 1.38 (s, 9H), 1.33-1.21 (m, 1H), 1.07-0.92 (m, 1H).

### Preparation of Compound 80

To a solution of compound 79 (130 mg, 333 umol, 1.00 eq) in DCM (1.00 mL) was added HCl/dioxane (4.00 M, 5.83 mL, 70.0 eq). The mixture was stirred at 25° C. for 2 hrs, concentrated under reduced pressure to give compound 80 (109 mg, crude, 96.8% yield, HCl) as a white solid. LC-MS (M+H)⁺: 291.1,

### Preparation of Compound 81

To a solution of compound 80 (108 mg, 330 umol, 1.00 eq, HCl) in MeOH (2.00 mL) was added AcONa (32.5 mg, 3% umol, 1.20 eq), NaBH₃CN (41.5 mg, 661 umol, 2.00 eq) and compound 7 (106 mg, 363 umol, 1.10 eq). The mixture was stirred at 25° C. for 3 hrs. The reaction mixture was quenched by addition water (3.00 mL) at 0° C., and then extracted with ethyl acetate (4.00 mL*3). The combined organic layers were washed with brine (4.00 mL), dried over Na₂SO₄, filtered and concentrated under reduced pressure to give a residue. The residue was purified by prep-HPLC (neutral condition; column: Waters Xbridge 150*25 mm*5 um: mobile phase: [water (10 mM NH₄HCO₃)−ACN]; B %: 52%-82%. 10 min). Compound 81 (91.0 mg, 92.5 umol, 27.9% yield, 57.4% purity) was obtained as a yellow oil. LC-MS (M+H)⁺: 565.6.

### Example 93: Preparation of (S)-3-(methyl(((R)-1-(3-(5,6,7,8-tetrahydro-1,8-naphthyridin-2-yl)propyl)piperidin-3-yl)methyl)amino)-3-phenylpropanoic acid hydrochloride (293)

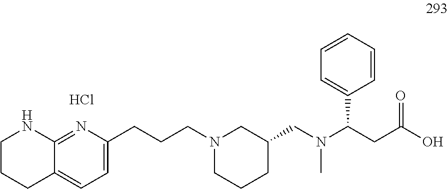 [SMILES: CN(C[C@@H]1CCCN(CCCc2ccc3c(n2)NCCC3)C1)[C@@H](CC(=O)O)c1ccccc1 ; Cl]

To a solution of compound 81 (81.0 mg, 143 umol, 1.00 eq) in H₂O (1.00 mL) was added HCl/dioxane (4.00 M, 2.51 mL, 70.0 eq). The mixture was stirred at 60° C. for 4 hrs and concentrated under reduced pressure to give a residue. The residue was purified by prep-HPLC (HCl condition: 3_Phenomenex Luna C18 75*30 mm*3 um; mobile phase: [water (0.05% HCl)−CAN]: B %: 4%-24%, 7 min) to provide compound 293 (56.5 mg, 110 umol) as a light-yellow oil. ¹H NMR (400 MHz, DMSO-d₆) δ 14.6-14.2 (m, 1H), 11.3-11.0 (m, 1H), 10.8 (s, 1H), 8.12 (s, 1H), 7.78-7.65 (m, 2H), 7.62 (d, J=7.6 Hz, 1H), 7.52-7.42 (m, 3H), 6.79-6.60 (m, 1H), 4.75 (s, 1H), 4.20-4.11 (m, 1H), 3.50-3.35 (m, 4H), 3.34-2.90 (m, 5H), 2.85-2.50 (m, 10H), 2.30-2.05 (m, 2H), 1.95-1.65 (m, 5H), 1.18-0.94 (m, 1H): LC-MS (M+H)⁺: 451.3.

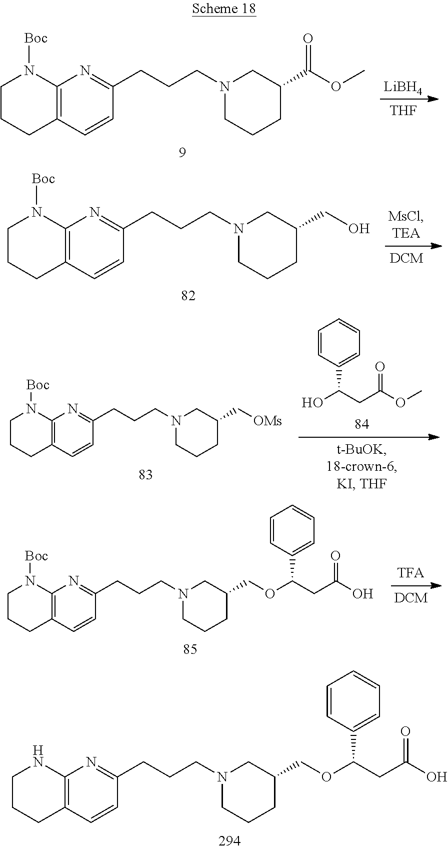

Scheme 18 illustrates the synthesis of compound 294.

### Preparation of Compound 82

To a solution of compound 9 (1.20 g, 2.87 mmol, 1.00 eq) in THF (15.0 mL) was added LiBH₄ (4 M, 934 uL, 1.30 eq) at 0° C. under N₂. The mixture was stirred at 25° C. for 5 hrs, quenched by saturated NH₄Cl solution 40.0 mL at 10° C., extracted with ethyl acetate (40.0 mL*3) and the combined organic layers were washed with brine 20.0 mL, dried over Na-2SO₄, filtered and concentrated under reduced pressure to give a residue. The residue was used to next step directly without any purification. Compound 82 (1.10 g, crude) was obtained as yellow oil. ¹H NMR (400 MHz, DMSO-d₆) δ 7.44-7.38 (m, 1H), 6.93-6.86 (m, 1H), 3.64-3.58 (m, 4H), 3.27-3.08 (m, 2H), 2.94 (d, J=11.6 Hz, 1H), 2.87-2.74 (m, 2H), 2.69 (t, J=6.4 Hz, 2H), 2.59 (t, J=7.6 Hz, 3H), 2.44-2.21 (m, 2H), 2.18-2.10 (m, 2H), 1.85-1.74 (m, 4H), 1.69-1.54 (m, 2H), 1.44 (s, 9H); LC-MS (M+H)⁺: 390.4.

### Preparation of Compound 83

To a solution of compound 82 (1.10 g, 2.82 mmol, 1.00 eq) in DCM (20.0 mL) was added MsCl (647 mg, 5.65 mmol, 437 uL, 2.00 eq) and TEA (857 mg, 8.47 mmol, 1.18 mL, 3.00 eq) at 0° C. The mixture was stirred at 25° C. for 2 hrs, quenched with saturated NaHCO₃ solution (30.0 mL), extracted with DCM (30.0 mL*3). Then the combined organic layers were dried over Na₂SO₄, filtered and concentrated under reduced pressure to give a residue. The residue was used in the next step directly without any purification. Compound 83 (1.20 g, crude) was obtained as yellow oil. ¹H NMR (400 MHz, CDCl₃;) δ 7.34-7.28 (m, 1H), 6.87-6.81 (m, 1H), 4.14-3.98 (m, 2H), 3.78-3.74 (m, 2H), 3.08 (d, J=9.6 Hz, 1H), 3.05-3.01 (m, 3H), 2.95-2.86 (m, 2H), 2.84-2.71 (m, 5H), 2.51-2.19 (m, 4H), 1.98-1.84 (m, 4H), 1.80-1.65 (m, 3H), 1.56-1.52 (m, 9H); LC-MS (M+H)⁺: 468.5.

### Preparation of Compound 85

To a solution of compound 83 (130 mg, 721 umol, 1.00 eq) and compound 84 (304 mg, 649 umol, 0.900 eq) in THF (10.0 mL) was added t-BuOK (89.0 mg, 794 umol, 1.10 eq), 18-crown-6 (153 mg, 577 umol, 0.800 eq), KI (35.9 mg, 216 umol, 0.300 eq). The mixture was stirred at 90° C. for 4 hrs, diluted with H₂O (20.0 mL) and extracted with EtOAc (20.0 mL*3). The combined organic layers were dried over Na₂SO₄, filtered and concentrated under reduced pressure to give a residue which was purified by Prep-HPLC (column: Waters Xbridge 150*25 mm*5 um; mobile phase: [water (10 mM NH₄HCO₃)−ACN]; B %: 49%-79%, 9 min) to provide compound 85 (15.0 mg, 27.9 umol, 3.87% yield) as a yellow oil. LC-MS (M+H)⁺: 538.5

### Example 94: (S)-3-phenyl-3-(((R)-1-(3-(5,6,7,8-tetrahydro-1,8-naphthyridin-2-yl)propyl)piperidin-3-yl)methoxy)propanoic acid (294)

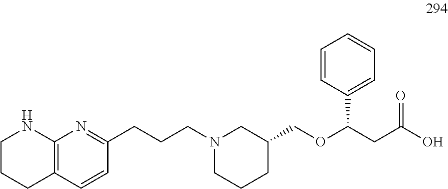 [SMILES: O=C(O)C[C@H](OC[C@@H]1CCCN(CCCc2ccc3c(n2)NCCC3)C1)c1ccccc1]

To a solution of compound 85 (10.0 mg, 18.6 umol, 1.00 eq) in DCM (0.500 mL) was added TFA (154 mg, 1.35 mmol, 0.100 mL, 72.6 eq). The mixture was stirred at 25° C. for 2 hrs, adjusted pH to about 7 with saturated NaHCO₃ solution at 0° C., diluted with H₂O (10.0 mL) and extracted with DCM (15.0 mL*3). The combined organic extracts were dried over Na₂SO₄, filtered and concentrated under reduced pressure to give a residue which was purified by Prep-HPLC (column: Waters Xbridge 150*25 mm*5 um; mobile phase: [water (10 mM NH₄HCO₃)−ACN]; B %: 31%-64%. 9 min) to yield compound 294 (4.10 mg, 8.79 umol, 47.3% yield) as a yellow gum. ¹H NMR (400 MHz, DMSO-d₆) δ 7.35-7.28 (m, 4H), 7.26-7.18 (m, 1H), 7.02 (d, J=7.2 Hz, 1H), 6.25 (d, J=7.2 Hz, 1H), 6.19 (s, 1H), 5.55-5.47 (m, 1H), 4.93 (t, J=6.8 Hz, 1H), 3.95-3.81 (m, 2H), 3.24-3.19 (m, 2H), 2.79 (brs, 2H), 2.65-2.55 (m, 4H), 2.42 (t, J=8.0 Hz, 3H), 2.11-1.96 (m, 1H), 1.84 (brs, 2H), 1.78-1.70 (m, 4H), 1.66-1.53 (m, 2H), 1.51-1.38 (m, 1H), 1.06-0.88 (m, 1H): LC-MS (M+H)⁺: 438.4,

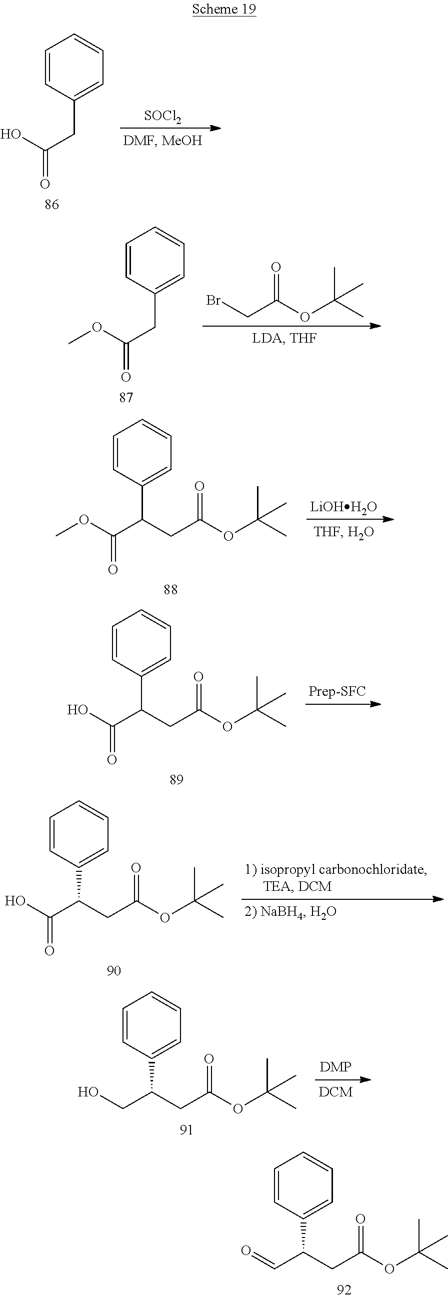

Scheme 19 illustrates the synthesis of intermediate 92.

### Preparation of Compound 87

To a solution of compound 86 (15.0 g, 110 mmol, 13.9 mL, 1.00 eq) in MeOH (50.0 mL) was added SOCl₂ (26.2 g. 220 mmol. 16.0 mL, 2.00 eq) at 0° C. Then DMF (805 mg, 11.0 mmol, 848 uL, 0.100 eq) was added and the mixture was stirred at 25° C. for 2 hrs, concentrated and purified by flash silica gel chromatography to obtain compound 87 (12.0 g, 79.9 mmol, 72.5% yield) as a yellow oil. ¹H NMR (400 MHz, CDCl₃) δ 7.33-7.29 (m, 2H), 7.25-7.24 (m, 3H), 3.67 (s, 3H), 3.62-3.61 (m, 2H), LC-MS (M+H)⁺: 151.1.

### Preparation of Compound 88

To a solution of compound 87 (5.00 g. 33.3 mmol, 4.67 mL, 1.00 eq) in THF (50.0 mL) was added dropwise LDA (2.00 M, 18.3 mL, 1.10 eq) at −78° C. under N₂ atmosphere, the mixture stirred at −78° C. for 2 hrs and then tert-butyl 2-bromoacetate (7.14 g, 36.6 mmol, 5.41 mL, 1.10 eq) was added dropwise. The mixture was then stirred at −78° C. for another 2 hrs, a solution of saturated NH₄Cl (30.0 mL) was added and the mixture was extracted with EtOAc (30.0 mL*3). The combined organic extracts were washed with H₂O (50.0 mL), dried over Na₂SO₄ and concentrated to give a residue which was purified by reversed-phase HPLC (0.1% NH₃·H₂O) to yield compound 88 (5.00 g, 18.9 mmol, 56.8% yield) as a yellow solid. ¹H NMR (400 MHz, CDCl₃) δ 7.33-7.28 (m, 5H), 4.03 (dd, J₁=10.0 Hz, J₂=5.6 Hz, 1H), 3.68 (s, 3H), 3.12 (dd, J₁=16.8 Hz, J₂=10.4 Hz, 1H), 2.61 (dd, J₁=16.4 Hz, J₂=5.6 Hz, 1H), 1.41 (s, 9H).

### Preparation of Compound 89

To a solution of compound 88 (5.00 g, 18.9 mmol, 1.00 eq) in THF (50.0 mL) was added a solution of LiOH·H₂O (1.59 g, 37.8 mmol, 2.00 eq) in H₂O (10.0 mL) and the mixture was stirred at 25° C. for 2 hrs. The mixture was concentrated, diluted with H₂O (30.0 mL) and extracted with EtOAc (30.0 mL*2). The pH of the aqueous phase was adjusted to about 4 by addition of a solution of HCl (1.00 M), which was then again extracted with EtOAc (50.0 mL*3). The combined organic extracts were washed with brine (50.0 mL), dried over Na₂SO₄ and concentrated to give compound 89 (4.00 g, 16.0 mmol, crude) as a yellow oil. ¹H NMR (400 MHz, CDCl₃) δ 7.34-7.30 (m, 5H), 4.05 (dd. J₁=10.0 Hz, J₂=5.2 Hz, 1H), 3.09 (dd, J₁=16.8 Hz, J₂=10.0 Hz, 1H), 2.63 (dd, J₁=16.8 Hz, J₂=5.6 Hz, 1H), 1.40 (s, 9H); LC-MS (M−H)⁺: 249.2.

### Preparation of Compound 90

The stereoisomers of compound 89 were separated by Prep-SFC (column: DAICEL CHIRALPAK IG (250 mm*30 mm, 10 um); mobile phase: [0.1% NH₃H₂O IPA]; B %: 30%-30%, 2.0; 40 min). Compound 90 (900 mg, 3.60 mmol, 45.0% yield) was obtained as yellow oil. LC-MS: (M−H)⁺: 249.1

### Preparation of Compound 91

To a solution of compound 90 (900 mg, 3.60 mmol, 1.00 eq) and isopropyl carbonochloridate (485 mg, 3.96 mmol, 549 uL, 1.10 eq) in DCM (10.0 mL) was added TEA (364 mg, 3.60 mmol, 500 uL, 1.00 eq) at 0° C. The mixture was stirred at 25° C. for 1 hrs, a solution of H₂O (30.0 mL) was added and the mixture extracted with DCM (20.0 mL*3). The combined organic extracts were washed with H₂O (30.0 mL), dried over Na₂SO₄ and concentrated to give a residue which was purified by Prep-TLC (Petroleum Ether: Ethyl Acetate=5:1) to provide compound 91 (500 mg, 2.12 mmol. 59.3% yield) as a light yellow oil. ¹H NMR (400 MHz, CDCl₃) δ 7.33-7.31 (m, 2H), 7.27-7.23 (m, 3H), 3.79-3.76 (m, 2H), 3.35-3.28 (m, 1H), 2.73 (dd, J₁=15.2 Hz, J₂=7.6 Hz, 1H), 2.58 (dd, J₁=15.2 Hz, J₂=7.6 Hz, 1H), 1.35 (s, 9H).

### Preparation of Compound 92

To a solution of compound 91 (200 mg, 846 umol, 1.00 eq) in DCM (5.00 mL) was added DMP (467 mg, 1.10 mmol, 341 uL, 1.30 eq) at 0° C. The mixture was stirred at 25° C. for 1 hrs, a solution of 20.0% Na₂SO₃ (20.0 mL) was added and the mixture was extracted with DCM (20.0 mL*3). The combined organic extracts were washed with H₂O (20.0 mL), dried over Na₂SO₄ and concentrated to give compound 92 (180 mg, crude) as yellow a solid. ¹H NMR (400 MHz, CDCl₃) δ 9.71 (s, 1H), 7.40-7.36 (m, 2H), 7.34-7.32 (m, 1H), 7.22-7.20 (m, 2H), 4.09 (dd, J₁=8.4 Hz, J: =2.0 Hz, 1H), 3.07 (dd, J₁=16.4 Hz, J₂=8.4 Hz, 1H), 2.56 (dd, J₁=16.4 Hz, J: =10.0 Hz, 1H), 1.40 (s, 9H).

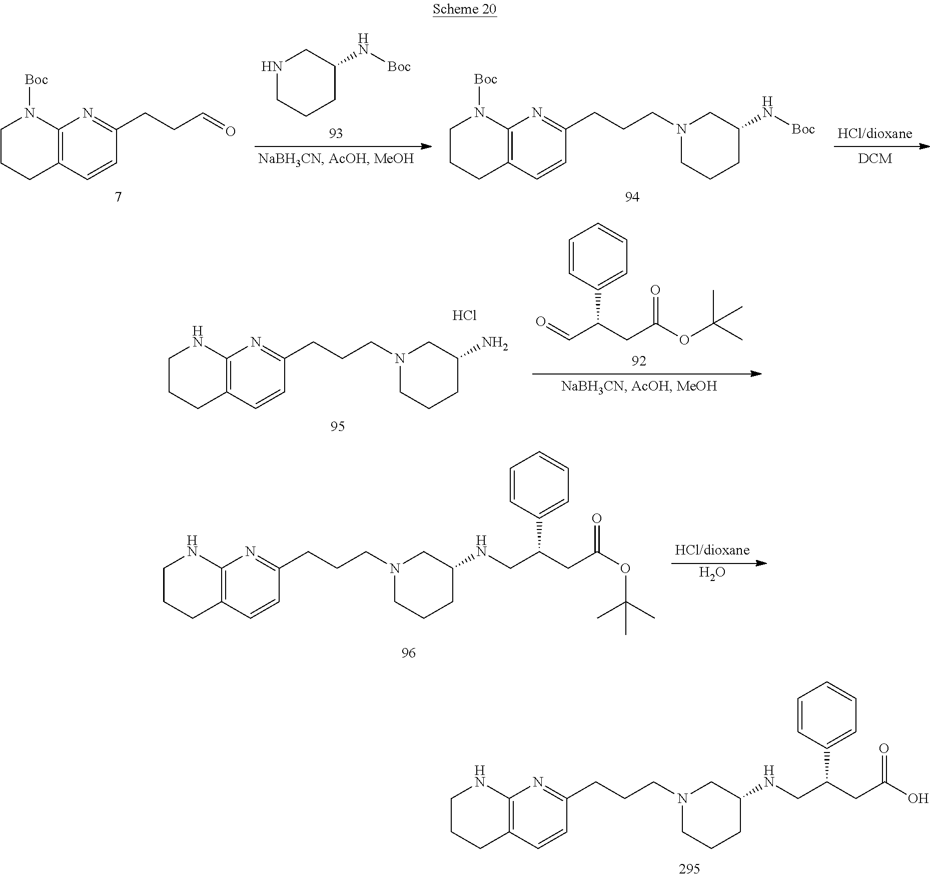

Scheme 20 illustrates the synthesis of compound 295.

### Preparation of Compound 94

To a solution of compound 7 (300 mg, 1.03 mmol, 1.00) eq) and compound 93 (228 mg, 1.14 mmol, 1.10 eq) in MeOH (3.00 mL) was added AcOH (6.20 mg, 103 umol. 5.91 uL, 0.100 eq). The mixture was stirred at 25° C. for 0.5 hrs, then NaBH₃CN (130 mg, 2.07 mmol, 2.00 eq) was added and the mixture was stirred at 25° C. for another 2 hrs, concentrated, diluted with H₂O (20.0 mL) and extracted with EtOAc (20.0 mL*3). The combined organic extracts were washed with brine (30.0 mL), dried over Na₂SO₄ and concentrated to give a residue, which was purified by Prep-HPLC (column: Waters Xbridge 150*25 mm*5 um; mobile phase: [water (10 mM NH₄HCO₃)−ACN]: B %: 51%-81%, 9 min) to yield compound 94 (250 mg, 527 umol, 51.0% yield) as a yellow oil. ¹H NMR (400 MHz, CDCl₃) δ 7.29 (d, J=7.6 Hz, 1H), 6.82 (d, J=7.6 Hz, 1H). 5.02 (br s, 1H), 3.76 (t, J=6.0 Hz, 3H), 2.72 (q, J=6.4 Hz, 4H), 2.47 (br s, 2H), 2.36 (t, J=6.4 Hz, 3H), 2.24-2.22 (m, 1H), 1.96-1.88 (m, 4H), 1.62 (s, 4H), 1.53 (s, 9H), 1.45 (s, 9H); LC-MS (M+H)⁺: 475.2.

### Preparation of Compound 95

To a solution of compound 94 (250 mg, 527 umol, 1.00 eq) in DCM (3.00 mL) was added HCl/dioxane (4.00 M, 1.00 mL, 7.59 eq) at 0° C. The mixture was stirred at 25° C. for 2 hrs and concentrated to give compound 95 (160 mg, 515 umol, crude) as a yellow solid.

### Preparation of Compound 96

To a solution of compound 95 (150 mg, 483 umol, 1.00 eq, HCl) and compound 92 (113 mg, 483 umol, 1.00 eq) in MeOH (5.00 mL) was added AcOH (2.90 mg, 48.3 umol, 2.76 uL, 0.100 eq). The mixture was stirred at 25° C. for 0.5 hrs, NaBH₃CN (45.5 mg, 724 umol, 1.50 eq) was added and the mixture was stirred at 25° C. for an additional 1.5 hrs, concentrated, diluted with H₂O (20.0 mL) and extracted with EtOAc (20.0 mL*3). The combined organic extracts were dried over Na₂SO₄ and concentrated to give a residue which was purified by Prep-HPLC (column: 3_Phenomenex Luna C18 75*30 mm*3 um; mobile phase: [water (0.05% HCl)−ACN]; B %: 12%-32%, 6.5 min) to provide compound 96 (150 mg, 304 umol, 63.1% yield) as a yellow solid. ¹H NMR (400 MHz, CDCl₃) δ 14.2 (br s, 1H), 8.00 (br s, 1H), 7.62 (d, J=6.8 Hz, 1H), 7.36-7.34 (m, 4H), 7.29-7.26 (m, 1H), 6.66 (d, J=6.8 Hz, 1H), 6.53-6.51 (m, 1H), 3.78-3.64 (m, 2H), 3.43-3.41 (m, 4H), 3.18-2.94 (m, 6H), 2.81-2.72 (m, 6H), 2.19-2.07 (m, 3H), 1.94-1.89 (m. 1H), 1.83-1.82 (m, 3H), 1.61-1.59 (m, 1H), 1.19 (s, 9H); LC-MS: (M+H)⁺: 493.2.

### Example 95: (S)-3-phenyl-4-(((R)-1-(3-(5,6,7,8-tetrahydro-1,8-naphthyridin-2-yl)propyl)piperidin-3-yl)amino)butanoic acid (295)

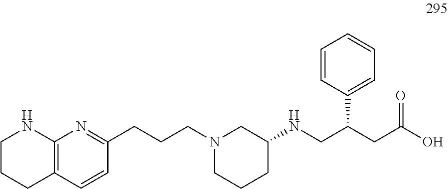 [SMILES: O=C(O)C[C@H](CN[C@@H]1CCCN(CCCc2ccc3c(n2)NCCC3)C1)c1ccccc1]

To a solution of compound 96 (40.0 mg, 81.2 umol, 1.00 eq) in H₂O (1.00 mL) was added HCl/dioxane (4.00 M, 1.00 mL, 49.3 eq). The mixture was stirred at 60° C. for 2 hrs, concentrated to give a residue which was purified by Prep-HPLC (column: Waters Xbridge 150*25 mm*5 um; mobile phase: [water (10 mM NH₄HCO₃)−ACN]: B %: 25%-55%, 10 min) to provide compound 295 (10.16 mg, 23.0 umol, 28.3% yield, 98.8% purity) as a light yellow oil. ¹H NMR (400 MHz, DMSO-d₆) δ 7.31-7.27 (m, 2H), 7.23-7.19 (m, 3H), 7.02 (d, J=7.2 Hz, 1H), 6.43 (s, 1H), 6.26-6.23 (m, 1H), 3.23 (s, 2H), 3.15-3.09 (m, 1H), 2.95-2.89 (m, 2H). 2.75-2.74 (m, 3H), 2.59 (d, J=6.0 Hz, 2H), 2.53-2.52 (m, 1H), 2.41-2.39 (m, 3H), 2.26 (t, J=7.2 Hz, 2H), 2.02-1.88 (m, 2H), 1.75-1.71 (m, 5H), 1.62-1.60 (m, 1H), 1.43-1.35 (m, 1H), 1.18-1.16 (m. 1H); LC-MS (M+H)⁺: 437.4.

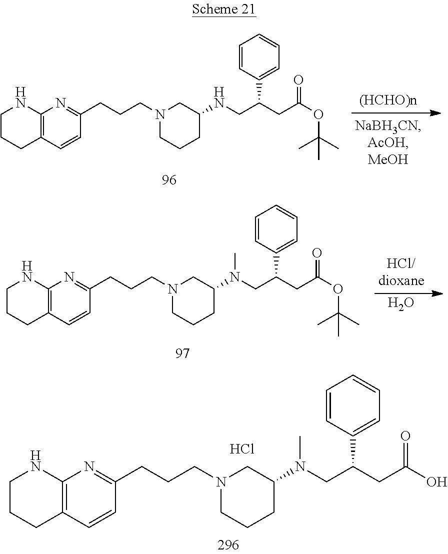 [SMILES: CC(C)(C)OC(=O)C[C@H](CN[C@@H]1CCCN(CCCc2ccc3c(n2)NCCC3)C1)c1ccccc1 ; CN(C[C@@H](CC(=O)OC(C)(C)C)c1ccccc1)[C@@H]1CCCN(CCCc2ccc3c(n2)NCCC3)C1 ; O ; CN(C[C@@H](CC(=O)O)c1ccccc1)[C@@H]1CCCN(CCCc2ccc3c(n2)NCCC3)C1 ; Cl]

Scheme 21 illustrates the synthesis of compound 296.

### Preparation of Compound 97

To a solution of compound 96 (40.0 mg, 81.2 umol, 1.00 eq) and (HCHO)n (20.0 mg) in MeOH (1.00 mL) was added AcOH (4.88 mg, 81.2 umol, 4.64 uL, 1.00 eq). The mixture was stirred at 25° C. for 0.5 hrs, NaBH₃CN (7.65 mg, 122 umol, 1.50 eq) was added and the mixture was stirred at 25° C. for another 1 hrs concentrated, diluted with H₂O (20.0 mL) and extracted with EtOAc (20.0 mL*5). The combined organic extracts were dried over Na₂SO₄ and concentrated to give a residue which was purified by Prep-TLC (Dichloromethane: Methanol=10:1, R_{f}═0.4) to provide compound 97 (35.0 mg, 69.1 umol, 85.1% yield) as a yellow oil. LC-MS (M+H)⁺: 507.5.

### Example 96: (S)-4-(methyl((R)-1-(3-(5,6,7,8-tetrahydro-1,8-naphthyridin-2-yl)propyl)piperidin-3-yl)amino)-3-phenylbutanoic acid (2%)

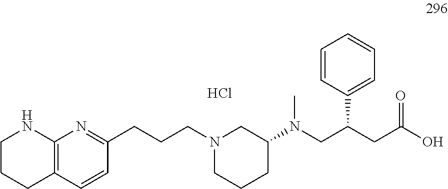 [SMILES: CN(C[C@@H](CC(=O)O)c1ccccc1)[C@@H]1CCCN(CCCc2ccc3c(n2)NCCC3)C1 ; Cl]

To a solution of compound 92 (35.0 mg, 69.1 umol, 1.00 eq) in H₂O (1.00 mL) was added HCl/dioxane (4.00 M, 1.00 mL, 57.9 eq) at 0° C. The mixture was stirred at 60° C. for 2 hrs and concentrated to give a residue which was purified by Prep-HPLC (column: 3_Phenomenex Luna C18 75*30 mm*3 um; mobile phase: [water (0.05% HCl)−ACN]; B %: 1%-21%, 6.5 min) to provide compound 2% (6.86 mg, 13.9 umol, 20.1% yield, 91.4% purity) as a light yellow oil. ¹H NMR (400 MHz, DMSO-d₆) δ 14.4 (br s, 1H), 11.5 (br s, 1H), 8.10 (s, 1H), 7.63 (d, J=7.2 Hz, 1H), 7.44 (d, J=6.8 Hz, 2H), 7.34 (t, J=7.2 Hz, 2H), 7.28-7.27 (m, 1H), 6.67 (d, J=7.2 Hz, 1H), 3.45-3.43 (m, 3H), 3.32-3.25 (m, 3H), 3.12-3.02 (m, 3H), 2.79-2.74 (m, 5H), 2.70-2.66 (m, 4H), 2.27-2.14 (m, 3H), 2.00-1.96 (m, 2H), 1.82-1.79 (m, 3H), 1.75 (s, 3H). LC-MS (M+H)⁺: 451.4.

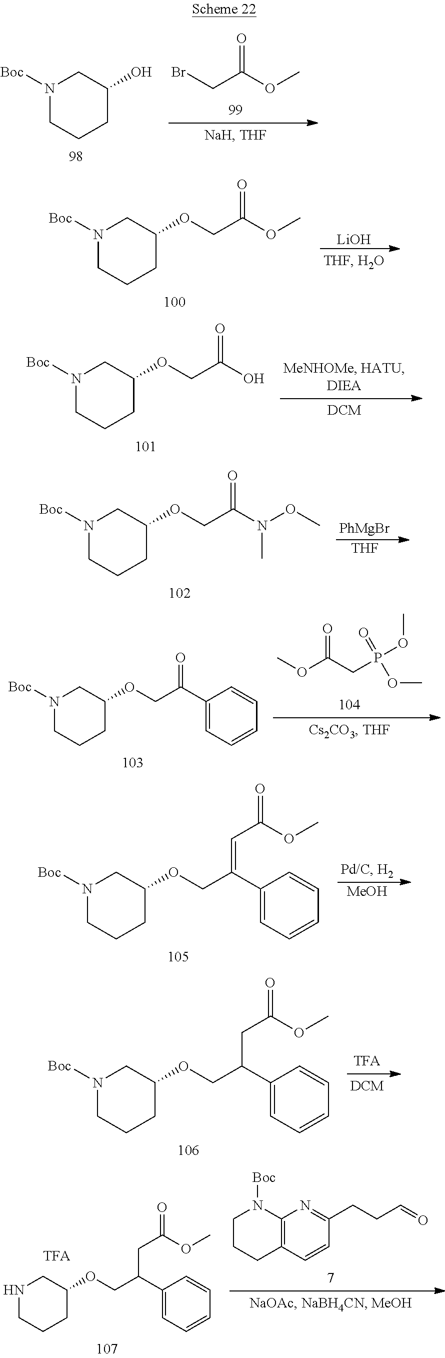

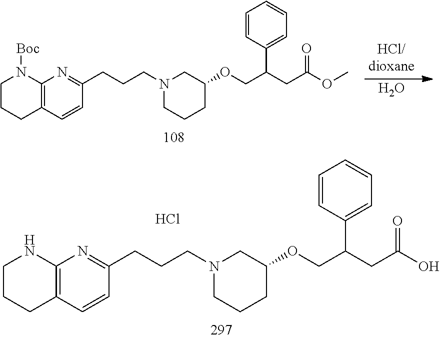 [SMILES: COC(=O)CC(CO[C@@H]1CCCN(CCCc2ccc3c(n2)N(C(=O)OC(C)(C)C)CCC3)C1)c1ccccc1 ; O ; O=C(O)CC(CO[C@@H]1CCCN(CCCc2ccc3c(n2)NCCC3)C1)c1ccccc1 ; Cl]

Scheme 22 illustrates the synthesis of compound 297.

### Preparation of Compound 100

To a solution of compound 98 (2.00 g. 9.94 mmol, 1.00 eq) and compound 99 (1.82 g, 11.9 mmol, 1.13 mL, 1.20 eq) in THF (20.0 mL) was added NaH (517 mg, 12.9 mmol, 60.0% purity, 1.30 eq) at 0° C. The mixture was stirred at 25° C. for 12 hrs, water (20.0 mL) was added, extracted with EtOAc 60.0 mL (20.0 mL*3). The combined extracts were dried over Na₂SO₄, concentrated to give a residue which was purified by flash silica gel chromatography to provide compound 100 (2.00 g, 7.32 mmol, 73.6% yield) as a yellow oil. LC-MS: (M+Na)⁺: 296.1.

### Preparation of Compound 101

To a solution of compound 100 (2.00 g, 7.32 mmol, 1.00 eq) in THF (10.0 mL) was added LiOH·H₂O (350 mg, 8.21 mmol, 1.12 eq) in H₂O (10.0 mL). The mixture was stirred at 25° C. for 4 hrs and concentrated to give compound 101 (1.90 g, crude) as an off-white liquid. LC-MS: (M-55)⁺: 204.1.

### Preparation of Compound 102

To a solution of compound 101 (1.90 g, 7.33 mmol, 1.00 eq) and MeNHOMe (1.07 g, 11.0 mmol, 1.50 eq, HCl) in DCM (30.0 mL) was added HATU (5.57 g, 14.7 mmol, 2.00 eq) and DIEA (3.79 g, 29.3 mmol, 5.11 mL, 4.00 eq). The mixture was stirred at 25° C. for 2 hrs, diluted with H₂O (50.0 mL), extracted with DCM (50.0 mL*5), dried over Na₂SO₄, filtered and concentrated give a residue which was purified by column chromatography (SiO₂, Petroleum ether: EtOAc=100:0 to 99:1) to yield compound 102 (1.30 g, 4.30 mmol, 58.7% yield) as an off-white liquid.

LC-MS: (M-99)⁺: 203.0.

### Preparation of Compound 103

To a solution of compound 102 (1.30 g, 4.30 mmol, 1.00 eq) in THF (15.0 mL) was added PhMgBr (2.90 M, 2.00 mL, 1.35 eq) at 0° C. The mixture was stirred at 25° C. for 3 hrs, water (20.0 mL) was added, extracted with EtOAc 90.0 mL (30.0 mL*3). The combined extracts were dried over Na₂SO₄, concentrated to yield compound 103 (1.35 g, 4.23 mmol, 98.3% yield) was obtained as yellow oil. ¹H NMR (400 MHz, CDCl₃) δ 7.94 (d, J=7.6 Hz, 2H), 7.61-7.57 (m, 1H), 7.49-7.57 (m, 2H), 3.77-3.73 (m, 2H), 3.61-3.56 (m, 1H), 3.49-3.45 (m, 1H), 3.16-3.08 (m. 2H), 1.89-1.84 (m, 2H), 1.82-1.75 (m, 1H), 1.46 (s, 9H); LC-MS (M+Na)⁺: 342.0.

### Preparation of Compound 105

To a solution of compound 103 (1.30 g, 4.07 mmol, 1.00 eq) and compound 104 (741.23 mg, 4.07 mmol, 588 uL, 1.00 eq) in THF (2.00 mL) was added Cs₂CO₃ (1.99 g, 6.11 mmol, 1.50 eq). The mixture was stirred at 25° C. for 2 hrs, concentrated to yield compound 105 (600 mg, 1.50 mmol, 37.0% yield, 94.1% purity) as a yellow oil. LC-MS (M+Na)⁺: 398.3.

### Preparation of Compound 106

To a solution of compound 105 (600 mg, 1.50 mmol, 94.1% purity. 1.00 eq) in MeOH (5.00 mL) was added Pd/C (100 mg, 10% purity) under N₂ and the suspension was degassed under vacuum and purged with H₂ several times. The reaction mixture was stirred under H₂ (15 psi) at 25° C. for 6 hrs and additional Pd/C (200 mg, 10% purity) was added and stirring was continued at 25° C. for another 12 hrs, filtered, concentrated to give compound 106 (400 mg, crude) as a yellow oil. LC-MS (M+Na)⁺: 400.2.

### Preparation of Compound 107

To a solution of compound 106 (200 mg, 530 umol, 1.00 eq) in DCM (2.00 mL) was added TFA (1.54 g, 13.5 mmol, 1.00 mL, 25.5 eq) at 0° C., the mixture was stirred at 25° C. for 2 hrs, concentrated to provide compound 107 (200 mg, crude, TFA) as a yellow oil. LC-MS (M+H)⁺: 277.9.

### Preparation of Compound 108

To a solution of compound 107 (50.0 mg, 128 umol, 1.00 eq, TFA) and compound 7 (42.0 mg, 145 umol, 1.13 eq) in MeOH (1.00 mL) was added NaOAc (14.2 mg, 173 umol, 1.35 eq). The mixture was stirred at 25° C. for 1 hrs, NaBH₃CN (13.6 mg, 217 umol, 1.69 eq) was added, stirring continued at 25° C. for an additional 1 hrs and concentrated to give a residue. The residue was purified by Prep-TLC (SiO₂, DCM: MeOH=10:1) to provide compound 108 as a yellow oil.

LC-MS (M+H)⁺: 552.2.

### Example 97: 3-phenyl-4-(((R)-1-(3-(5,6,7,8-tetrahydro-1,8-naphthyridin-2-yl)propyl)piperidin-3-yl)oxy)butanoic acid hydrochloride (297)

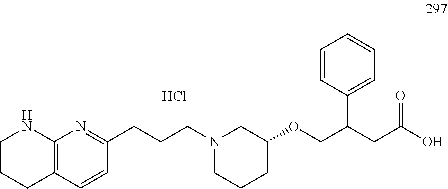 [SMILES: O=C(O)CC(CO[C@@H]1CCCN(CCCc2ccc3c(n2)NCCC3)C1)c1ccccc1 ; Cl]

To a solution of compound 108 (40.0 mg, 56.7 umol, 78.2% purity. 1.00 eq) in H₂O (2.00 mL) was added HCl/dioxane (4 M. 2.00 mL, 141 eq). The mixture was stirred at 60° C. for 1 hr., concentrated under vacuum to give a residue which was purified by Prep-HPLC (column: Phenomenex luna C18 150*25 mm*10 um; mobile phase: [water (0.05% HCl)−ACN]; B %: 4%-34%, 11 min) to yield compound 297 (9.97 mg, 22.0 umol, 38.8% yield, 96.6% purity, HCl) as a yellow gum. ¹H NMR (400 MHz, DMSO-d₆) δ 10.80 (br s, 1H), 8.15-8.06 (m, 1H), 7.63 (d, J=7.6 Hz, 1H), 7.29-7.18 (m, 5H), 6.68-6.65 (m, 1H), 3.80-3.66 (m, 3H), 3.65-3.56 (m, 2H), 3.50-3.43 (m, 3H), 3.34-3.22 (m, 2H), 3.05-3.04 (m, 2H), 3.03 (br s, 1H), 2.79-2.66 (m, 5H), 2.12 (br s, 3H), 1.83-1.75 (m, 4H), 1.66-1.43 (m, 1H): LC-MS (M+H)⁺: 438.4.

Stereoisomers of compound 297 were purified by Prep-SFC (column: DAICEL CHIRALPAK IG (250 mm*30 mm, 10 um); mobile phase: [0.1% NH₃·H₂O IPA]; B %: 40%-40%, 8; 60 min). Compound 297-A (14.49 mg, 32.8 umol, 47.8% yield, 98.9% purity) was obtained as yellow solid. Compound 297-B (21.47 mg, 48.3 umol, 70.5% yield, 98.5% purity) was obtained as off-white solid.

297-A: ¹H NMR (400 MHz, CDCl₃) δ 11.12 (br s. 1H). 7.32-7.28 (m, 3H), 7.26 (br s, 1H), 7.22-7.20 (m, 2H), 6.28 (d, J=7.2 Hz, 1H), 4.07 (br s, 1H), 3.90-3.88 (m, 1H), 3.72-3.71 (m, 2H), 3.64-3.61 (m, 1H), 3.45 (t, J=5.6 Hz, 2H), 3.10-3.07 (m, 1H), 2.73-2.70 (m, 4H), 2.61-2.58 (m, 2H), 2.47-2.40 (m, 2H), 2.26-2.14 (m, 2H), 1.91-1.69 (m, 7H), 1.30-1.26 (m, 1H): LC-MS (M+H)⁺: 438.2.

297-B: ¹H NMR (400 MHz, CDCl₃) δ 11.30 (br s, 1H), 7.31-7.28 (m, 2H), 7.26-7.24 (m, 2H), 7.21-7.19 (m, 2H), 6.26 (d, J=7.2 Hz, 1H), 4.14 (br s, 1H), 4.00 (dd, J₁=11.2 Hz, J₂=3.6 Hz, 1H), 3.78 (t, J=10.8 Hz, 1H), 3.47-3.44 (M. 3H). 3.36-3.33 (m, 1H), 2.99-2.92 (m, 1H). 2.73-2.70 (m, 4H), 2.55-2.43 (m, 5H), 2.08 (br s, 1H), 1.91-1.88 (m, 3H), 1.70-1.61 (m, 4H), 1.28-1.26 (m, 1H); LC-MS (M+H)⁺: 438.2.

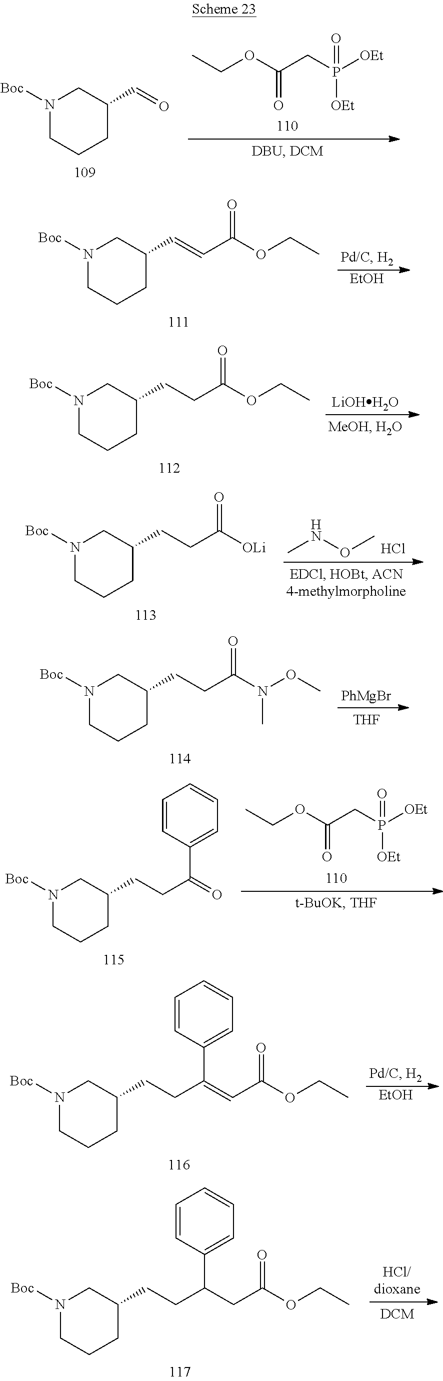

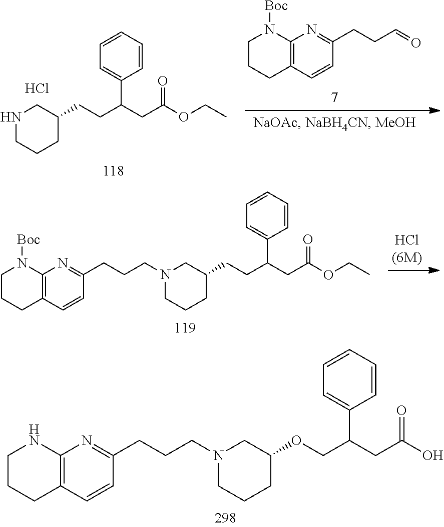 [SMILES: CCOC(=O)CC(CC[C@@H]1CCCNC1)c1ccccc1 ; Cl ; CC(C)(C)OC(=O)N1CCCc2ccc(CCC=O)nc21 ; CCOC(=O)CC(CC[C@@H]1CCCN(CCCc2ccc3c(n2)N(C(=O)OC(C)(C)C)CCC3)C1)c1ccccc1 ; O=C(O)CC(CO[C@@H]1CCCN(CCCc2ccc3c(n2)NCCC3)C1)c1ccccc1]

Scheme 23 illustrates the synthesis of compound 298.

### Preparation of Compound 111

To a solution of compound 109 (6.31 g, 28.1 mmol, 5.58 mL, 1.20 eq) in DCM (40.0 mL) was added DBU (7.14 g, 46.9 mmol, 7.07 mL, 2.00 eq) at 0° C., the mixture was stirred at 0° C. for 1 hr. Then compound 110 (5.00 g, 23.4 mmol, 1.00 eq) was added, the mixture was stirred at 25° C. for 3 hrs, diluted with H₂O (20.0 mL) and extracted with DCM (20.0 mL*3). The combined organic extracts were washed with brine (30.0 mL*2), dried over Na₂SO₄, filtered, concentrated to give a residue, which was purified by flash silica gel chromatography (ISCO®; 20 g SepaFlash® Silica Flash Column, Eluent of 0˜50% EtOAc: Petroleum ether gradient @ 20 mL/min) to provide compound 111 (4.00 g, 14.1 mmol, 60.2% yield) as a colorless oil. ¹H NMR (400 MHz, CDCl₃) δ 6.84 (dd, J₁=15.6 Hz, J₂=6.8 Hz, 1H), 5.86 (dd, J₁=16.0 Hz, J₂=1.2 Hz, 1H), 4.19 (q, J=6.8 Hz, 2H), 4.03-3.82 (m, 2H), 2.91-2.56 (m, 2H), 2.38-2.22 (m, 1H), 1.94-1.83 (m, 1H), 1.74-1.60 (m, 2H), 1.46 (s, 9H), 1.42-1.33 (m, 1H), 1.29 (t, J=6.8 Hz, 3H).

### Preparation of Compound 112

To solution of compound 111 (4.00 g, 14.1 mmol, 1.00 eq) in EtOH (40.0 mL) was added Pd/C (400 mg, 10% purity) under N₂. The suspension was degassed under vacuum, purged with H₂ 3 times, stirred under H₂ (45 psi) at 25° C. for 12 hrs, filtered and concentrated to yield compound 112 (3.00 g, 10.5 mmol, 74.5% yield) as a colorless oil which was used in the next step directly without any purification. LC-MS (M-99)⁺: 186.3: ¹H NMR (400 MHz, CDCl₃) δ 4.13 (q, J=6.8 Hz, 2H), 4.04-3.68 (m, 2H), 2.88-2.72 (m, 1H), 2.62-2.38 (m, 1H), 2.33 (t, J=8.0 Hz, 2H), 1.84-1.75 (m, 1H), 1.72 (s, 1H). 1.66-1.59 (m, 1H), 1.58-1.51 (m, 1H), 1.50-1.47 (m, 1H), 1.45 (s, 9H), 1.43-1.34 (m, 1H), 1.25 (t, J=87.2 Hz, 3H), 1.17-1.01 (m, 1H).

### Preparation of Compound 113

To a solution of compound 112 (2.00 g, 7.01 mmol, 1.00 eq) in MeOH (20.0 mL) was added LiOH·H₂O (382 mg, 9.11 mmol, 1.30 eq) in H₂O (1.00 mL). The mixture was stirred at 25° C. for 8 hrs, diluted with H₂O (20.0 mL) and extracted with DCM (30.0 mL*2). The aqueous layer was concentrated under reduced pressure to give a residue which was used in the next step directly without any purification. Compound 113 (1.90 g, 6.76 mmol, 96.4% yield, LiOH) was obtained as a white solid. ¹H NMR (400 MHz, DMSO_d₆) δ 3.93-3.63 (m, 2H), 2.78-2.63 (m, 1H), 1.88 (t, J=7.2 Hz, 2H), 1.76-1.64 (m, 1H), 1.60-1.50 (n, 1H), 1.41 (s, 1H), 1.38 (s, 9H), 1.35-1.21 (m, 4H), 1.09-0.92 (m, 1H); LC-MS (M-55)⁺: 202.1.

### Preparation of Compound 114

To a solution of compound 113 (1.90 g, 6.76 mmol, 1.00 eq, LiOH), N,O-dimethylhydroxylamine (1.32 g, 13.5 mmol, 2.00 eq. HCl) in acetonitrile (20.0 mL) was added EDCI (1.94 g, 10.1 mmol, 1.50 eq), HOBt (1.37 g, 10.1 mmol, 1.50 eq), 4-methylmorpholine (4.10 g, 40.5 mmol, 4.46 mL, 6.00 eq). The mixture was stirred at 25° C. for 2 hrs, concentrated, diluted with H₂O (30.0 mL) and extracted with EtOAc (30.0 mL*3). The combined organic extracts were washed with brine (30.0 mL*2) dried over Na₂SO₄, filtered, concentrated under reduced pressure to give a residue, which was purified by flash silica gel chromatography (ISCO®; 20 g SepaFlash® Silica Flash Column, Eluent of 0-50% EtOAc: Petroleum ether gradient @ 20 mL/min). The eluate was further purified by Prep-HPLC (column: Phenomenex Gemini−NX C18 75*30 mm*3 um: mobile phase: [water (10 mM NH₄HCO₃)−ACN]; B %: 35%-55%, 8 min) to yield compound 114 (1.30 g, 4.33 mmol, 64.1% yield) as a yellow oil. ¹H NMR (400 MHz, CDCl₃) δ 4.07-3.79 (m, 2H), 3.68 (s, 3H), 3.18 (s, 3H), 2.82-2.69 (m, 1H), 2.59-2.35 (m, 3H), 1.88-1.53 (m, 5H), 1.48 (s, 1H), 1.45 (s, 9H), 1.17-1.03 (m, 1H); LC-MS (M-99)⁺: 201.2.

### Preparation of Compound 115

To a solution of compound 114 (1.00 g, 3.33 mmol, 1.00 eq) in THF (30.0 mL) was added PhMgBr (3.00 M, 4.44 mL, 4.00 eq) in diethyl ether at 0° C. under N₂. The mixture was stirred at 0° C. for 1 hrs, at 25° C. for 6 hrs, quenched by NH₄Cl (30.0 mL) and extracted with DCM (30.0 mL*3). The organic extracts were washed with brine (30.0 mL*2), dried over Na₂SO₄, filtered, concentrated to give a residue, which was purified by flash silica gel chromatography (ISCO®; 20 g SepaFlash® Silica Flash Column, Eluent of 0-50% EtOAc: Petroleum ether gradient @ 20 mL/min) to yield compound 115 (700 mg, 2.21 mmol, 66.2% yield) as a white solid. ¹H NMR (400 MHz, CDCl₃) δ 7.99-7.94 (m, 2H), 7.60-7.53 (m, 1H), 7.47 (t, J=8.0 Hz, 2H), 4.14-3.85 (m, 2H), 3.15-2.95 (m, 2H), 2.88-2.73 (m, 1H), 2.66-2.41 (m, 1H), 1.92-1.86 (m, 1H), 1.75-1.62 (m, 3H), 1.58-1.48 (m, 2H), 1.46 (s, 9H), 1.22-1.09 (m, 1H); LC-MS (M-99)⁺: 218.2.

### Preparation of Compound 116

To a solution of compound 115 (700 mg, 3.15 mmol, 625 uL, 2.00 eq) in THF (10.0 mL) was added t-BuOK (442 mg, 3.94 mmol, 2.50 eq) at 0° C., and the mixture was stirred at 0° C. for 1 hr. Then compound 110 (500 mg, 1.58 mmol, 1.00 eq) was added. The mixture was stirred at 90° C. for 12 hrs, diluted with H₂O (20.0 mL) and extracted with EtOAc (20.0 mL*3). The combined organic extracts were washed with brine (30.0 mL*1), dried over Na₂SO₄, filtered, concentrated under reduced pressure to give a residue which was purified by Prep-TLC (SiO₂, Petroleum ether: EtOAc=5:1) to provide compound 116 (180 mg, 464 umol, 29.5% yield) was obtained as colorless oil. ¹H NMR (400 MHz, CDCl₃) δ 7.46-7.34 (m, 5H), 6.03 (s, 1H), 4.22 (q, J=7.2 Hz, 2H), 4.00-3.87 (m, 2H), 3.26-3.12 (m, 1H), 3.11-2.99 (m, 1H), 2.77-2.67 (m, 1H), 2.50-2.46 (m, 1H), 1.93-1.82 (m, 1H), 1.66-1.60 (m, 2H), 1.53-1.48 (m, 1H), 1.45 (s, 9H), 1.44-1.41 (m, 1H), 1.41-1.37 (m, 1H), 1.32 (t, J=7.2 Hz, 3H) LC-MS (M-99)⁺: 288.6.

### Preparation of Compound 117

To solution of compound 116 (180 mg. 464 umol, 1.00 eq) in EtOH (20.0 mL) was added Pd/C (30.0 mg, 10% purity) under N₂. The suspension was degassed under vacuum, purged with H₂ 3 times, stirred under H₂ (45 psi) at 25° C. for 4 hrs, filtered concentrated to provide compound 117 (100 mg, 257 umol. 55.3% yield) was obtained as colorless oil, which was used directly in the next step without any purification. LC-MS (M-99)⁺: 290.5.

### Preparation of Compound 118

To a solution of compound 117 (100 mg, 257 umol, 1.00 eq) in DCM (3.0) mL) was added HCl/dioxane (4.00 M, 1.28 mL, 20.0 eq). The mixture was stirred at 25° C. for 5 hrs, concentrated to provide compound 118 (80.0 mg, crude, HCl) was obtained as light-yellow oil which was used to next step directly without any purification. LC-MS (M+H)⁺: 290.7.

### Preparation of Compound 119

To a solution of compound 118 (80.0 mg, 245 umol, 1.00 eq, HCl) in MeOH (2.0) mL) was added NaOAc (40.3 mg, 491 umol, 2.00 eq), compound 7 (85.5 mg, 295 umol, 1.20 eq). The mixture was stirred at 25° C. for 1 hrs, then NaBH₃CN (30.8 mg, 491 umol, 2.00 eq) followed by additional stirring at 25° C. for 3 hrs, concentrated, diluted with H₂O (20.0 mL) and extracted with EtOAc (20.0 mL*3), The combined organic extracts were washed with saturated NaHCO₃(30.0 mL*1), brine (30.0 mL*1), dried over Na₂SO₄, filtered, concentrated to give a residue, which was purified by Prep-TLC (SiO₂, DCM: MeOH=10:1) to provide compound 119 (50.0 mg, 88.7 umol, 36.1% yield) as a yellow oil. LC-MS (M+H)⁺: 564.5.

### Example 98: 3-phenyl-5-((R)-1-(3-(5,6,7,8-tetrahydro-1,8-naphthyridin-2-yl)propyl)piperidin-3-yl)pentanoi c acid (298)

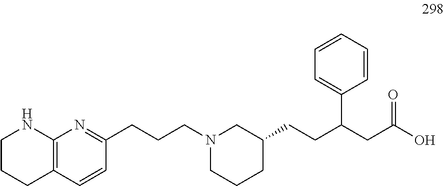 [SMILES: O=C(O)CC(CC[C@@H]1CCCN(CCCc2ccc3c(n2)NCCC3)C1)c1ccccc1]

A solution of compound 119 (50.0 mg, 88.7 umol, 1.00 eq) in HCl (6.00 M. 2.00 mL, 135 eq) was stirred at 60° C. for 2 hrs and washed with DCM (10.0 mL*3), The aqueous phase was adjusted pH to −7 with NaHCO₃ solution, concentrated give a residue, which was purified by Prep-SFC. Sample preparation: Add IPA and CH₂Cl₂ 100 mL into sample Instrument: Waters 150 SFC Mobile Phase: 40% IPA (0.1% NH₃·H₂O) in Supercritical CO₂ Flow Rate: 90 g/min Cycle Time: 4.5 min, total time: 40 min Single injection volume: 4.5 mL Back Pressure: 100 bar to keep the CO₂ in Supercritical flow).

Compound 298-A (14.24 mg, 31.4 umol, 35.5% yield, 96.2% purity) was obtained as yellow gum. Compound 296-B (16.86 mg, 38.6 umol, 43.5% yield, 99.7% purity) was obtained as yellow gum.

298-A: ¹H NMR (400 MHz, CDCl₃) δ 11.13 (s, 1H), 7.25-7.20 (m, 2H), 7.19-7.07 (m, 4H), 6.18 (d, J=7.2 Hz, 1H), 3.69-3.58 (m, 1H), 3.37 (br s, 2H), 3.26 (t, J=10.8 Hz, 1H), 3.13-3.05 (m, 1H), 2.75 (d, J=7.2 Hz, 1H), 2.62 (t, J=6.0 Hz, 3H), 2.54-2.40 (m, 3H), 2.36-2.29 (m, 1H), 2.21-2.11 (m, 2H), 2.08-1.99 (m, 1H), 1.94-1.78 (m, 4H), 1.72-1.61 (m. 1H), 1.57-1.50 (m, 1H), 1.47-1.39 (m, 2H), 1.25-1.12 (m, 1H), 1.07-0.91 (m, 2H), 0.82-0.69 (m, 1H); LC-MS (M+H)⁺: 436.4.

298-B: ¹H NMR (400 MHz, DMSO_d₆) δ 14.60-13.92 (m, 1H), 10.75-9.89 (m, 1H), 8.03 (s, 1H), 7.61 (d, J=7.2 Hz, 1H), 7.31-7.16 (m, 4H), 6.64 (d, J=7.2 Hz, 1H), 3.46-3.41 (m, 3H), 3.29-3.23 (m, 2H), 3.04-3.86 (m, 3H), 2.77-2.69 (m, 4H), 2.64-2.55 (m, 1H), 2.44 (d, J=8.4 Hz, 1H), 2.15-1.96 (m, 2H), 1.85-1.73 (m, 4H), 1.69-1.51 (m, 2H), 1.35-1.18 (m, 3H), 1.16-1.02 (m, 1H), 0.97-0.80 (m, 2H): LC-MS (M+H)⁺: 436.6.

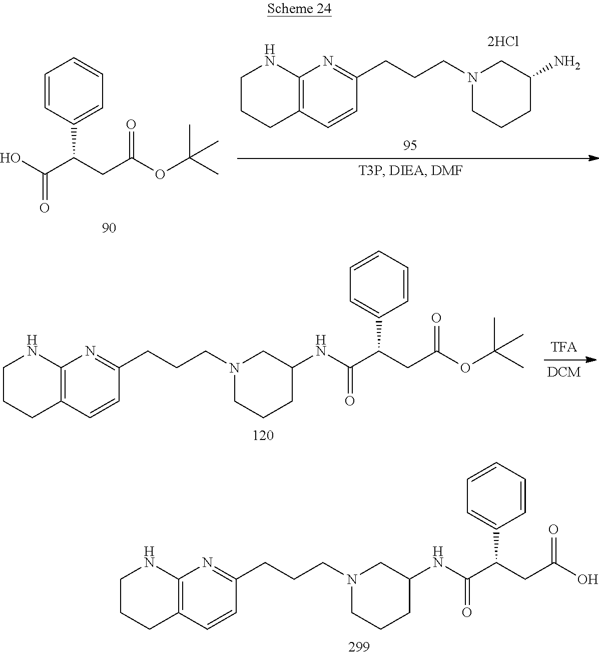

Scheme 24 illustrates the synthesis of compound 299.

### Preparation of Compound 120

To a solution of compound 90 (50.0 mg, 129 umol, 89.9% purity, 1.00 eq, 2HCl) and compound 95 (26.0 mg, 104 umol, 0.8 eq) in DCM (2.00 mL) was added T3P (165 mg, 259 umol, 154 uL, 50.0% purity, 2.00 eq) and DIEA (83.6 mg, 647 umol, 113 uL, 5.00 eq). The mixture was stirred at 25° C. for 3 hrs, concentrated to give a residue which was purified by Prep-TLC (SiO₂, DCM: MeOH=10:1) to yield compound 120 (40.0 mg, 79.0 umol, 61.0% yield) as a yellow oil.

LC-MS (M+H)⁺: 507.5.

### Example 99: (S)-4-oxo-3-phenyl-4-(((R)-1-(3-(5,6,7,8-tetrahydro-1,8-naphthyridin-2-yl)propyl)piperidin-3-yl)amino)butanoic acid (299)

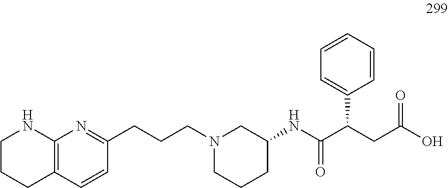 [SMILES: O=C(O)C[C@H](C(=O)N[C@@H]1CCCN(CCCc2ccc3c(n2)NCCC3)C1)c1ccccc1]

To a solution of compound 120 (20.0 mg, 39.5 umol, 1.00 eq) in DCM (1.00 mL) was added TFA (3.08 g, 27.0 mmol, 2.00 mL, 684 eq) at 0° C. The reaction mixture was stirred at 25° C. for 2 hrs, concentrated to give a residue which was purified by Prep-HPLC (column: Waters xbridge 150*25 mm 10 um; mobile phase: [water (10 mM NH₄HCO₃)−ACN]; B %: 13%-43%, 11 min) to yield compound 299 (3.01 mg, 6.68 umol, 16.9% yield, 100% purity) as a white solid. ¹H NMR (400 MHz, DMSO-d₆) δ 7.84 (d, J=8.4 Hz, 1H), 7.33-7.25 (m, 3H), 7.22-7.12 (m, 2H), 6.32 (d, J=7.2 Hz, 1H), 3.90 (dd. J₁=10.0 Hz, J₂=4.4 Hz, 1H), 3.72-3.69 (m, 1H), 3.27-3.24 (M, 4H), 2.94 (dd, J₁=16.8 Hz, J₂=10.0 Hz, 1H), 2.62 (t, J=6.0 Hz, 2H), 2.44-2.29 (m, 5H), 2.25-2.19 (m, 1H), 2.28-2.18 (m, 2H), 1.78-1.63 (m, 4H), 1.48-1.31 (m, 2H), 1.17-1.12 (m, 1H): LC-MS (M+H)⁺: 451.4.

The following compounds, set forth in Table 6, were prepared according to the general procedures provided in Scheme 24 or analogous procedures thereto.

TABLE 6

| Compound | |
|---|---|
| No. | ¹H NMR Data |
| 414 | (400 MHz, CDCl₃) δ 10.42 (s, 1H), 8.24 (d, J = 8.4 Hz, 1H), 7.28 (s, 2H), |
| | 7.27 (s, 2H), 7.23 (d, J = 7.6 Hz, 1H), 7.21-7.15 (m, 1H), 6.28 (d, J = 7.2 |
| | Hz, 1H), 4.57-4.55 (m, 1H), 3.60-3.53 (m, 2H), 3.44 (t, J = 5.6 Hz, 2H), |
| | 3.25 (br t, J = 6.8 Hz 1H), 3.06 (d, J = 10.0 Hz, 1H), 2.86-2.82 (m, 1H), |
| | 2.74-2.64 (m, 8H), 2.40 (br s, 1H), 2.27-2.20 (m, 2H), 1.94-1.88 (m, |
| | 3H), 1.81-1.74 (m, 2H), 1.72-1.67 (m, 2H). |
| 415 | (400 MHz, DMSO-d₆) δ 7.86 (d, J = 7.2 Hz, 1H), 7.25-7.18 (m, 4H), 7.16- |
| | 7.11 (m, 1H), 7.02 (d, J = 7.2 Hz, 1H), 6.28 (s, 1H), 6.24 (d, J = 7.2 Hz, |
| | 1H), 4.05-4.00 (m, 1H), 3.45-3.43 (m, 2H), 3.24-3.21 (m, 2H), 2.61- |
| | 2.55 (m, 3H), 2.53-2.51 (m, 1H), 2.45-2.39 (m, 3H), 2.36-2.23 (m, |
| | 5H), 2.01-1.94 (m, 2H), 1.77-1.71 (m, 2H), 1.58-1.50 (m, 2H), 1.48- |
| | 1.41 (m, 1H), 1.38-1.30 (m, 2H). |
| 416 | (400 MHz, CDCl₃) δ 10.93-10.53 (m, 1H), 8.37-7.92 (m, 1H), 7.29 (s, |
| | 1H), 7.26-7.22 (m, 3H), 7.22-7.17 (m, 1H), 6.39-6.26 (m, 1H), 4.71- |
| | 4.49 (m, 1H), 3.80-3.73 (m, 1H), 3.67-3.59 (m, 2H), 3.54-3.48 (m, |
| | 2H), 3.46-3.39 (m, 2H), 2.88-2.80 (m, 1H), 2.79-2.77 (m, 1H), 2.75- |
| | 2.70 (m, 3H), 2.67 (d, J = 6.0 Hz, 2H), 2.64-2.61 (m, 1H), 2.60-2.50 (m, |
| | 1H), 2.44-2.34 (m, 1H), 2.18-2.09 (m, 3H), 2.07-1.98 (m, 1H), 1.95- |
| | 1.87 (m, 2H). |
| 417 | (400 MHz, DMSO-d₆) δ 8.13 (s, 1H), 7.95 (dd, J₁ = 26.8 Hz, J₂ = 6.4 Hz, |
| | 1H), 7.28-7.14 (m, 5H), 7.12-7.05 (m, 1H), 6.44-6.34 (m, 1H), 6.28 |
| | (dd, J₁ = 7.2 Hz, J₂ = 5.2 Hz, 1H), 3.49-3.44 (m, 2H), 3.25 (d, J = 3.2 Hz, |
| | 2H), 3.13-3.05 (m, 2H), 2.69-2.53 (m, 4H), 2.47-2.44 (m, 2H), 2.42- |
| | 2.32 (m, 3H), 2.21-2.09 (m, 2H), 1.87-1.72 (m, 5H), 1.54-1.39 (m, 1H). |
| 418 | (400 MHz, DMSO-d₆ + D₂O) δ 7.34-7.20 (m, 5H), 7.06-7.00 (m, 1H), |
| | 6.28-6.21 (m, 1H), 4.24-4.08 (m, 1H), 3.58-3.53 (m, 1H), 3.41-3.31 |
| | (m, 3H), 3.22 (br s, 2H), 3.17-3.08 (m, 1H), 2.62-2.57 (m, 2H), 2.46- |
| | 2.40 (m, 2H), 2.23-2.11 (m, 3H), 2.07-1.93 (m, 3H), 1.87-1.69 (m, 6H). |
| 419 | (400 MHz, DMSO-d₆) δ 8.00 (dd, J₁ = 17.6 Hz, J₂ = 6.4 Hz, 1H), 7.35- |
| | 7.21 (m, 5H), 7.03 (t, J = 6.8 Hz, 1H), 6.38 (d, J = 50.8 Hz, 1H), 6.24 (q, |
| | J = 4.0 Hz, 1H), 4.22-4.11 (m, 1H), 3.57 (q, J = 6.0 Hz, 1H), 3.46-3.42 |
| | (m, 2H), 3.35-3.32 (m, 1H), 3.23 (br s, 2H), 3.17-3.14 (m, 1H), 2.60 (t, |
| | J = 5.6 Hz, 2H), 2.46-2.39 (m, 2H), 2.23-2.12 (m, 3H), 2.06-1.92 (m, |
| | 3H), 1.87-1.63 (m, 6H). |
| 420 | (400 MHz, DMSO-d₆) δ 7.95 (br d, J = 7.8 Hz, 1H), 7.33-7.20 (m, 5H), |
| | 7.06 (d, J = 7.2 Hz, 1H), 6.92 (s, 1H), 6.25 (d, J = 7.2 Hz, 1H), 4.17-4.08 |
| | (m, 1H), 3.44 (t, J = 6.8 Hz, 1H), 3.24 (t, J = 4.4 Hz, 2H), 2.65-2.58 (m, |
| | 3H), 2.52 (d, J = 2.0 Hz, 1H), 2.45-2.34 (m, 4H), 2.34-2.26 (m, 2H), |
| | 2.19-2.09 (m, 1H), 2.06-1.98 (m, 3H), 1.87-1.79 (m, 1H), 1.77-1.71 |
| | (m, 2H), 1.69-1.62 (m, 1H), 1.56-1.47 (m, 2H), 1.46-1.37 (m, 2H). |
| 421 | (400 MHz, DMSO-d₆) δ 7.96 (br d, J = 6.8 Hz, 1H), 7.31-7.19 (m, 5H), |
| | 7.01 (d, J = 7.6 Hz, 1H), 6.36 (s, 1H), 6.24 (d, J = 7.6 Hz, 1H), 4.10 (t, J = |
| | 4.0 Hz, 1H), 3.41 (t, J = 7.6 Hz, 1H), 3.22 (br t, J = 5.2 Hz, 2H), 2.63- |
| | 2.57 (m, 3H), 2.52 (d, J = 1.6 Hz, 1H), 2.44-2.32 (m, 6H), 2.18-2.08 (m, |
| | 1H), 2.06-1.94 (m, 3H), 1.85-1.77 (m, 1H), 1.77-1.70 (m, 2H), 1.61- |
| | 1.46 (m, 3H), 1.45-1.36 (m, 2H). |
| 422 | (400 MHz, DMSO-d₆) δ 8.01 (dd, J₁ = 18.1 Hz, J₂ = 6.6 Hz, 1H), 7.33- |
| | 7.22 (m, 5H), 7.03 (t, J = 5.6 Hz, 1H), 6.45-6.28 (m, 1H), 6.25-6.23 (m, |
| | 1H), 4.24-4.10 (m, 1H), 3.59-3.55 (m, 1H), 3.47-3.40 (m, 3H), 3.23 (s, |
| | 2H), 3.17-3.12 (m, 1H), 2.60 (t, J = 5.1 Hz, 2H), 2.45-2.41 (m, 2H), |
| | 2.22-2.15 (m, 3H), 2.06-1.91 (m, 3H), 1.87-1.69 (m, 6H). |
| 423 | (400 MHz, DMSO-d₆) δ 8.01 (dd, J₁ = 19.1 Hz, J₂ = 6.6 Hz, 1H), 7.39- |
| | 7.18 (m, 5H), 7.04-7.01 (m, 1H), 6.41 (s, 1H), 6.26-6.23 (m, 1H), 4.24- |
| | 4.13 (m, 1H), 3.58-3.54 (m, 1H), 3.50-3.46 (m, 1H), 3.42-3.33 (m, |
| | 2H), 3.26-3.21 (m, 2H), 3.15-3.09 (m, 1H), 2.63-2.56 (m, 2H), 2.43 (q, |
| | J = 6.8 Hz, 2H), 2.24-2.09 (m, 3H), 2.09-1.92 (m, 3H), 1.90-1.63 (m, 6H). |
| 424 | (400 MHz, DMSO-d₆) δ 8.20-8.12 (m, 1H), 7.89 (d, J = 7.6 Hz, 2H), 7.67 |
| | (s, 2H), 7.66-7.61 (m, 2H), 7.00 (d, J = 7.2 Hz, 1H), 6.26-6.20 (m, 2H), |
| | 4.12-4.04 (m, 2H), 3.69 (d, J = 2.4 Hz, 1H), 3.24-3.20 (m, 3H), 2.68- |
| | 2.61 (m, 2H), 2.37-2.32 (m, 3H), 2.25-2.14 (m, 2H), 2.09-1.93 (m, |
| | 4H), 1.83-1.62 (m, 7H), 1.61 (s, 6H), 1.45-1.32 (m, 3H). |
| 424 | (400 MHz, DMSO-d₆) δ 7.91 (br d, J = 5.6 Hz, 1H), 7.34-7.29 (m, 2H), |
| | 7.28-7.22 (m, 3H), 7.09-7.04 (m, 1H), 6.98-6.92 (m, 1H), 6.25 (d, J = |
| | 7.2 Hz, 1H), 4.14-4.08 (m, 1H), 3.45 (br s, 2H), 3.24 (br s, 1H), 2.69- |
| | 2.64 (m, 1H), 2.62-2.58 (m, 4H), 2.44-2.39 (m, 3H), 2.34-2.26 (m, |
| | 3H), 2.14-2.07 (m, 1H), 2.03-2.00 (m, 2H), 1.86-1.79 (m, 1H), 1.79- |
| | 1.72 (m, 2H), 1.70-1.61 (m, 1H), 1.56-1.47 (m, 2H), 1.45-1.39 (m, 2H). |
| 425 | (400 MHz, CDCl₃) δ 10.89 (br s, 1H), 8.22 (d, J = 7.6 Hz, 1H), 7.23 (d, J = |
| | 7.2 Hz, 1H), 6.59 (d, J = 7.6 Hz, 1H), 6.25 (d, J = 7.2 Hz, 1H), 4.51-4.42 |
| | (m, 3 H), 3.47 (t, J = 4.8 Hz, 2H), 3.13-3.08 (m, 1H), 3.02 (d, J = 9.6 Hz, |
| | 1H), 2.90-2.82 (m, 1H), 2.71 (t, J = 6.0 Hz, 3H), 2.66-2.58 (m, 1H), |
| | 2.45-2.42 (m, 1H), 2.38-2.31 (m, 1H ), 2.27-2.16 (m, 3H), 2.04-1.98 |
| | (m, 3H), 1.94-1.85 (m, 3H), 1.76-1.71 (m, 6H), 1.65-1.57 (m, 10H). |
| 426 | (400 MHz, DMSO-d₆) δ 7.94-7.92 (m, 1H), 7.21 (d, J = 6.8 Hz, 1H), 7.03 |
| | (d, J = 7.2 Hz, 1H), 6.49 (s, 1H), 6.25 (d, J = 7.2 Hz, 1H), 4.09 (br s, 1H), |
| | 3.99-3.93 (m, 1H), 3.22 (br s, 2H), 2.83 (d, J = 6.4 Hz, 1H), 2.60 (t, J = |
| | 6.0 Hz, 3H), 2.43 (t, J = 7.6 Hz, 2H), 2.11-1.98 (m, 4H), 1.91-1.84 (m, |
| | 2H), 1.77-1.71 (m, 3H), 1.63-1.55 (m, 11H), 1.54-1.44 (m, 9H). |
| 428 | (400 MHz, DMSO-d₆) δ 8.10 (dd, J₁ = 25.6 Hz, J₂ = 6.8 Hz, 1H), 7.35 (t, |
| | J = 6.8 Hz, 1H), 7.05 (d, J = 7.2 Hz, 1H), 6.51 (s, 1H), 6.26 (dd, J₁ = 7.2 Hz, |
| | J₂ = 2.8 Hz, 1H), 4.28-4.12 (m, 1H), 4.12-4.02 (m, 1H), 3.58 (dd, J₁ = |
| | 10.8 Hz, J₂ = 6.4 Hz, 1H), 3.46-3.39 (m, 4H), 3.34-3.32 (m, 2H), 3.24 |
| | (s, 1H), 3.20-3.12 (m, 2H), 2.61 (t, J = 6.4 Hz, 2H), 2.45 (d, J = 5.2 Hz, |
| | 2H), 2.25-2.16 (m, 2H), 2.14-2.08 (m, 2H), 2.06-1.91 (m, 2H), 1.83- |
| | 1.75 (m, 4H), 1.64-1.59 (m, 5H), 1.58-1.55 (m, 1H), 1.55-1.49 (m, 5H). |
| 429 | (400 MHz, DMSO-d₆) δ 8.14-8.02 (m, 1H), 7.35 (t, J = 6.8 Hz, 1H), 7.05 |
| | (d, J = 7.2 Hz, 1H), 6.51 (s, 1H), 6.26 (dd, J₁ = 7.2 Hz, J₂ = 2.8 Hz, 1H), |
| | 4.28-4.12 (m, 1H), 4.12-4.02 (m, 1H), 3.58 (dd, J₁ = 10.8 Hz, J₂ = 6.4 |
| | Hz, 1H), 3.46-3.39 (m, 4H), 3.34-3.32 (m, 2H), 3.24 (s, 1H), 3.20-3.12 |
| | (m, 2H), 2.61 (t, J = 6.4 Hz, 2H), 2.45 (d, J = 5.2 Hz, 2H), 2.25-2.16 (m, |
| | 2H), 2.14-2.08 (m, 2H), 2.06-1.91 (m, 2H), 1.83-1.75 (m, 4H), 1.64- |
| | 1.59 (m, 5H), 1.58-1.55 (m, 1H), 1.55-1.49 (m, 5H). |
| 430 | (400 MHz, DMSO-d₆) δ 14.30-14.15 (m, 1H), 10.99-10.79 (m, 1H), |
| | 8.50-8.28 (m, 1H), 8.07 (br d, J = 1.4 Hz, 1H), 7.61 (br d, J = 7.2 Hz, |
| | 1H), 7.48-7.37 (m, 1H), 6.67-6.57 (m, 1H), 4.44-4.27 (m, 1H), 4.17- |
| | 4.09 (m, 1H), 3.47-3.46 (m, 1H), 3.16-3.11 (m, 2H), 2.75-2.68 (m, |
| | 4H), 2.13 (br s, 4H), 2.03-1.96 (m, 2H), 1.82 (br d, J = 4.8 Hz, 4H), 1.69 |
| | (br d, J = 1.6 Hz, 5H), 1.65-1.59 (m, 6H), 1.52 (br s, 7H), 1.32-1.22 (m, |
| | 2H). |
| 431 | (400 MHz, DMSO-d₆) δ 8.11 (d, J = 6.4 Hz, 1H), 7.19 (d, J = 6.8 Hz, 1H), |
| | 7.08 (d, J = 7.6 Hz, 1H), 7.03 (s, 1H), 6.26 (d, J = 7.6 Hz, 1H), 4.14 (s, |
| | 1H), 4.03-3.95 (m, 1H), 3.26-3.22 (m, 2H), 2.90-2.83 (m, 1H), 2.71- |
| | 2.65 (m, 2H), 2.60 (t, J = 6.0 Hz, 3H), 2.45 (d, J = 5.6 Hz, 2H), 2.13-2.03 |
| | (m, 4H), 1.93-1.82 (m, 2H), 1.77-1.71 (m, 2H), 1.64-1.57 (m, 8H), |
| | 1.56-1.46 (m, 11H). |
| 432 | (400 MHz, DMSO-d₆) δ 8.09 (dd, J₁ = 14.8 Hz, J₂ = 6.8 Hz, 1H), 7.34 (d, |
| | J = 7.6 Hz, 1H), 7.04 (d, J = 7.2 Hz, 1H), 6.42 (br d, J = 9.6 Hz, 1H), 6.24 |
| | (dd, J₁ = 3.6 Hz, J₂ = 3.6 Hz, 1H), 4.26-4.11 (m, 1H), 4.10-4.02 (m, |
| | 1H), 3.58 (dd, J₁ = 6.0 Hz, J₂ = 4.4 Hz, 1H), 3.43-3.38 (m, 2H), 3.25- |
| | 3.22 (m, 2H), 3.16-3.12 (m, 1H), 2.60 (t, J = 6.2 Hz, 2H), 2.47-2.41 (m, |
| | 2H), 2.23-2.07 (m, 4H), 2.05-1.90 (m, 2H), 1.84-1.69 (m, 6H), 1.64- |
| | 1.48 (m, 13H). |
| 433 | (400 MHz, DMSO-d₆) δ 8.09 (dd, J₁ = 25.6 Hz, J₂ = 6.8 Hz, 1H), 7.33- |
| | 7.30 (m, 1H), 7.05 (d, J = 7.3 Hz, 1H), 6.61 (br s, 1H), 6.25 (dd, J₁ = 7.2 |
| | Hz, J₂ = 3.2 Hz, 1H), 4.25-4.13 (m, 1H), 4.09-4.05 (m, 1H), 3.57 (dd, J₁ = |
| | 10.4 Hz, J₂ = 6.2 Hz, 1H), 3.46-3.33 (m, 2H), 3.24 (br t, J = 5.3 Hz, |
| | 2H), 3.19-3.10 (m, 1H), 2.60 (t, J = 6.4 Hz, 2H), 2.46-2.42 (m, 2H), |
| | 2.23-2.16 (m, 2H), 2.13-2.06 (m, 2H), 2.03-1.87 (m, 2H), 1.86-1.69 |
| | (m, 6H), 1.65-1.47 (m, 13H). |
| 434 | (400 MHz, DMSO-d₆) δ 8.20-8.12 (m, 1H), 7.89 (d, J = 7.6 Hz, 2H), 7.67 |
| | (s, 2H), 7.66-7.61 (m, 2H), 7.00 (d, J = 7.2 Hz, 1H), 6.26-6.20 (m, 2H), |
| | 4.12-4.04 (m, 2H), 3.69 (d, J = 2.4 Hz, 1H), 3.24-3.20 (m, 3H), 2.68- |
| | 2.61 (m, 2H), 2.37-2.32 (m, 3H), 2.25-2.14 (m, 2H), 2.09-1.93 (m, |
| | 4H), 1.83-1.62 (m, 7H), 1.61 (s, 6H), 1.45-1.32 (m, 3H). |
| 435 | (400 MHz, DMSO-d₆) δ 8.19-8.12 (m, 1H), 7.91-7.87 (m, 2H), 7.75- |
| | 7.70 (m, 1H), 7.70-7.66 (m, 1H), 7.64 (d, J = 7.6 Hz, 2H), 7.00 (d, J = 7.2 |
| | Hz, 1H), 6.27-6.20 (m, 2H), 4.13-4.05 (m, 2H), 3.73-3.66 (m, 1H), |
| | 3.24-3.17 (m, 3H), 2.59 (br d, J = 6.4 Hz, 3H), 2.43-2.37 (m, 3H), 2.35- |
| | 2.29 (m, 3H), 2.23-2.18 (m, 1H), 2.07-1.98 (m, 3H), 1.86-1.79 (m, |
| | 2H), 1.75-1.65 (m, 4H), 1.59-1.43 (m, 6H), 1.43-1.37 (m, 2H). |
| 436 | (400 MHz, DMSO-d₆) δ 8.22-8.12 (m, 1H), 7.96 (d, J = 7.6 Hz, 1H), 7.90- |
| | 7.83 (m, 2H), 7.76-7.68 (m, 1H), 7.66-7.59 (m, 2H), 7.07 (d, J = 7.2 |
| | Hz, 2H), 6.26 (d, J = 7.2 Hz, 1H), 4.19-4.02 (m, 4H), 3.45-3.41 (m, 1H), |
| | 3.24 (t, J = 5.2 Hz, 2H), 3.20-3.14 (m, 1H), 2.93 (d, J = 6.0 Hz, 1H), 2.79- |
| | 2.67 (m, 2H), 2.60 (t, J = 6.0 Hz, 4H), 2.46-2.40 (m, 2H), 2.18-2.07 |
| | (m, 3H), 1.96-1.84 (m, 2H), 1.81-1.70 (m, 4H), 1.69-1.57 (m, 3H), |
| | 1.56-1.40 (m, 4H). |
| 437 | (400 MHz, DMSO-d₆) δ 8.23-8.17 (m, 1H), 7.96 (br d, J = 7.6 Hz, 1H), |
| | 7.87 (d, J = 7.4 Hz, 2H), 7.76-7.68 (m, 1H), 7.67-7.59 (m, 2H), 7.07 (br |
| | d, J = 7.4 Hz, 1H), 6.26 (d, J = 7.4 Hz, 1H), 4.20-4.02 (m, 4H), 3.45- |
| | 3.41 (m, 1H), 3.25-3.13 (m, 3H), 2.93 (br d, J = 5.0 Hz, 1H), 2.80-2.58 |
| | (m, 6H), 2.47-2.41 (m, 2H), 2.18-2.04 (m, 3H), 1.99-1.85 (m, 2H), |
| | 1.82-1.70 (m, 4H), 1.69-1.57 (m, 3H), 1.56 (br s, 4H). |
| 438 | (400 MHz, DMSO-d₆) δ 8.23-8.07 (m, 1H), 7.93-7.79 (m, 3H), 7.76- |
| | 7.68 (m, 1H), 7.66-7.59 (m, 2H), 7.06-6.99 (m, 1H), 6.43 (s, 1H), 6.26- |
| | 6.20 (m, 1H), 4.21-4.09 (m, 2H), 3.97 (s, 1H), 3.61-3.57 (m, 2H), 3.26- |
| | 3.20 (m, 3H), 3.19-3.10 (m, 3H), 2.59 (t, J = 6.0 Hz, 2H), 2.45-2.39 (m, |
| | 2H), 2.23-2.07 (m, 4H), 2.03-1.89 (m, 2H), 1.85-1.69 (m, 8H), 1.63- |
| | 1.46 (m, 2H). |
| 439 | (400 MHz, DMSO-d₆) δ 8.14 (br d, J = 7.4 Hz, 1H), 8.06 (dd, J₁ = 26.4 |
| | Hz, J₂ = 5.2 Hz, 1H), 7.88 (d, J = 7.8 Hz, 2H), 7.75-7.69 (m, 1H), 7.66- |
| | 7.60 (m, 2H), 7.04 (br d, J = 7.2 Hz, 1H), 6.65-6.56 (m, 1H), 6.25 (dd, J₁ = |
| | 7.2 Hz, J₂ = 3.2 Hz, 1H), 4.26-4.21 (m, 1H), 4.18-4.14 (m, 1H), 4.11 |
| | (br s, 1H), 3.58 (dd, J₁ = 10.4 Hz, J₂ = 6.4 Hz, 1H), 3.46-3.38 (m, 3H), |
| | 3.23 (br t, J = 5.2 Hz, 2H), 3.19-3.12 (m, 2H), 2.59 (br t, J = 6.0 Hz, 2H), |
| | 2.47-2.41 (m, 2H), 2.23-2.12 (m, 4H), 2.06-1.92 (m, 2H), 1.87-1.62 |
| | (m, 9H), 1.52-1.45 (m, 1H). |
| 440 | (400 MHz, DMSO-d₆) δ 8.18-8.01 (m, 2H), 7.88 (d, J = 8.0 Hz, 2H), 7.73- |
| | 7.70 (m, 1H), 7.67-7.58 (m, 2H), 7.07 (d, J = 7.6 Hz, 1H), 6.76 (br d, J = |
| | 18.6 Hz, 1H), 6.26 (dd, J₁ = 6.8 Hz, J₂ = 1.2 Hz, 1H), 4.25-4.09 (m, |
| | 3H), 3.60-3.56 (m, 2H), 3.43-3.38 (m, 3H), 3.32-3.29 (m, 1H), 3.24- |
| | 3.12 (m, 4H), 2.60 (br t, J = 6.0 Hz, 2H), 2.24-2.11 (m, 4H), 2.06-1.90 |
| | (m, 2H), 1.87-1.59 (m, 9H), 1.52-1.39 (m, 1H). |
| 441 | (400 MHz, DMSO-d₆) δ 8.15-8.01 (m, 2H), 7.86 (dd, J₁ = 7.2 Hz, J₂ = |
| | 1.2 Hz, 2H), 7.76-7.59 (m, 3H), 7.10-7.02 (m, 1H), 6.70 (d, J = 35.2 Hz, |
| | 1H), 6.27-6.24 (m, 1H), 4.27-4.09 (m, 3H), 3.60-3.56 (m, 1H), 3.48- |
| | 3.38 (m, 4H), 3.24 (br t, J = 4.8 Hz, 2H), 3.19-3.15 (m, 2H), 2.60 (br t, J = |
| | 5.2 Hz, 2H), 2.46-2.42 (m, 2H), 2.25-2.10 (m, 4H), 2.05-1.91 (m, |
| | 2H), 1.84-1.72 (m, 7H), 1.67-1.57 (m, 1H), 1.54-1.44 (m, 1H). |
| 520 | (400 MHz, CDCl₃) δ 11.0-10.8 (m, 1H), 8.31-8.15 (m, 1H), 7.31-7.29 |
| | (m, 2H), 7.26-7.19 (m, 4H), 6.33-6.30 (m, 1H), 4.72-4.52 (m, 2H), |
| | 3.78-3.43 (m, 7H), 2.89-2.49 (m, 9H), 2.44-2.32 (m, 1H), 2.20-1.98 |
| | (m, 4H), 1.94-2.88 (m, 2H). |
| 521 | (400 MHz, CDCl₃) δ 10.7 (br s, 1H), 8.40 (br s, 1H), 8.06 (d, J = 8.0 Hz, |
| | 1H), 7.77 (d, J = 6.0 Hz, 1H), 7.29-7.27 (m, 1H), 7.26-7.16 (m, 5H), |
| | 6.31 (dd, J₁ = 7.2 Hz, J₂ = 3.6 Hz, 1H), 4.60-4.48 (m, 1H), 3.73-3.38 (m, |
| | 7H), 2.82-2.55 (m, 9H), 2.48-2.29 (m, 1H), 2.14-1.97 (m, 4H), 1.91- |
| | 1.88 (m, 2H). |
| 522 | (400 MHz, CDCl₃) δ 10.8 (br s, 1H), 8.08-8.00 (m, 1H), 7.52-7.28 (m, |
| | 3H), 7.27-7.18 (m, 4H), 6.33 (d, J = 7.2 Hz, 1H), 4.69-4.57 (m, 1H), |
| | 3.77-3.45 (m, 7H), 2.82-2.67 (m, 8H), 2.61-2.49 (m, 1H), 2.35-2.34 |
| | (m, 1H), 2.09-2.00 (m, 4H), 1.94-1.89 (m, 2H). |

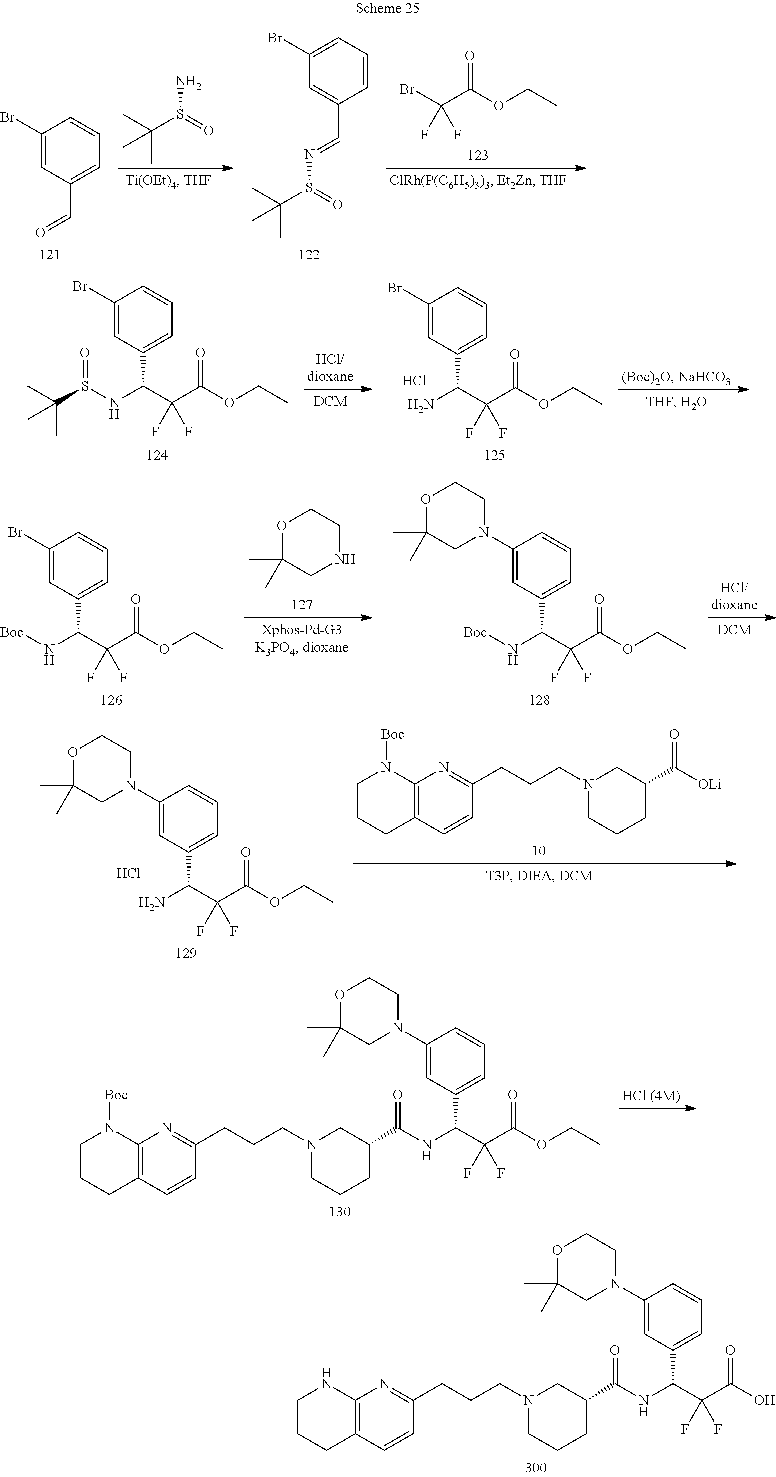

Scheme 25 illustrates the synthesis of compound 300.

### Preparation of Compound 122

To a solution of compound 121 (12.0 g, 64.9 mmol, 7.55 mL, 1.00 eq) and (R)-2-methylpropane-2-sulfinamide (15.7 g, 130 mmol, 2.00 eq) in THF (50.0 mL) was added Ti(OEt)₄ (14.8 g, 64.9 mmol, 13.5 mL, 1.00 eq). The mixture was stirred at 66° C. for 5 hrs, diluted with H₂O (50.0 ml), filtered and extracted with ethyl acetate (50.0 mL*3). The combined organic extracts were washed with brine (60.0 mL), dried over Na₂SO₄, concentrated to give a residue which was purified by column chromatography (SiO₂, Petroleum ether: Ethyl acetate=50:1 to 5:1, Petroleum ether: Ethyl acetate=5:1, R_{f}=0.50) to provide compound 122 (10.0 g, 34.7 mmol, 53.5% yield) as a yellow oil. ¹H NMR (400 MHz, DMSO-d₆) δ 8.55 (s. 1H), 8.13 (t, J=2.0 Hz, 1H), 7.97-7.94 (m, 1H), 7.79-7.78 (m, 1H), 7.51 (t, J=7.6 Hz, 1H), 1.19 (s, 9H); LC-MS (M+H)⁺: 288.1.

### Preparation of Compound 124

To a solution of compound 122 (5.00 g, 17.4 mmol, 1.00 eq), ClRh(P(C₆H₅)₃)₃ (642 mg, 694 umol, 0.0400 eq) and compound 123 (7.04 g, 34.7 mmol, 4.46 mL, 2.00 eq) in THF (50.0 mL) was added dropwise Et₂Zn (1.00 M. 34.7 mL, 2.00 eq) at −78° C. under N₂. The mixture was stirred at 0° C. for 1 hrs under N₂, a solution of NH₄Cl (50.0 mL) was added, extracted with ethyl acetate (50.0 mL*3). The combined organic extracts were washed with brine (50.0 mL*2), dried over Na₂SO₄ and concentrated to give a residue which was purified by column chromatography (SiO₂, Petroleum ether: Ethyl acetate=30:1 to 3:1, Petroleum ether: Ethyl acetate=3:1, R_{f}=0.30, I₂) to provide compound 124 (3.00 g, 7.28 mmol, 41.9% yield) was obtained as yellow oil. ¹H NMR (400 MHz, DMSO-d₆) δ 7.90 (s, 1H), 7.60-7.56 (m, 2H), 7.37-7.33 (m, 1H), 6.35 (d, J=10.8 Hz, 1H), 5.04-4.94 (m, 1H), 4.30-4.23 (m, 2H), 1.27-1.23 (m, 3H), 1.10 (s, 9H): LC-MS (M+H)⁺: 413.9.

### Preparation of Compound 125

To a solution of compound 124 (3.00 g, 7.28 mmol, 1.00 eq) in DCM (0.500 mL) was added HCl/dioxane (4.00 M, 30.00 mL, 16.49 eq) at 0° C. The mixture was stirred at 25° C. for 1 hrs and concentrated to compound 125 (2.50 g, crude, HCl) as a yellow solid. LC-MS (M+H)⁺: 309.4.

### Preparation of Compound 126

To a solution of compound 125 (2.50 g, 7.26 mmol, 1.00 eq, HCl) in THF (20.0 mL) and H₂O (5.00 mL) was added (Boc)₂O (2.38 g, 10.9 mmol, 2.50 mL, 1.50 eq) and NaHCO₃(1.83 g, 21.8 mmol, 847 uL, 3.00 eq). The mixture was stirred at 25° C. for 12 hrs, diluted with H₂O (30.0 mL) and extracted with ethyl acetate (20.0 mL*3). The combined organic extracts were washed with brine (30.0 mL*2), dried over Na₂SO₄ and concentrated to give a residue which was purified by column chromatography (SiO₂, Petroleum ether: Ethyl acetate=100:1 to 5:1, R_{f}=0.50, I₂) to yield compound 126 (2.00 g, 4.90 mmol, 67.5% yield) was obtained as yellow solid. ¹H NMR (400 MHz, CDCl₃) δ 7.51-7.49 (m, 2H), 7.31-7.23 (m, 2H), 5.37 (s, 2H), 4.32-4.26 (m, 2H), 1.43 (s, 9H), 1.30 (t, J=7.2 Hz, 3H); LC-MS (M-55)⁺: 351.9.

### Preparation of Compound 128

To a solution of compound 126 (300 mg, 735 umol, 1.00 eq) and compound 127 (169 mg, 1.47 mmol, 2.00 eq) in dioxane (3.00 mL) was added K₃PO₄ (468 mg, 2.20 mmol, 3.00 eq) and Xphos-Pd-G3 (187 mg, 220 umol, 0.300 eq) under N₂. The mixture was stirred at 90° C. for 3 hrs, diluted with H₂O (20.0 mL) and extracted with ethyl acetate (20.0 mL*3). The combined organic extracts was washed with brine (30.0 mL*2), dried over Na₂SO₄, concentrated to give a residue which was purified by Prep-TLC (Petroleum ether: Ethyl acetate=5:1, R_{f}═0.45) to yield compound 128 (90.0 mg, 203 umol, 27.7% yield) was obtained as yellow oil. LC-MS (M+H)⁺: 443.1.

### Preparation of Compound 129

To a solution of compound 128 (90.0 mg, 203 umol, 1.00 eq) in DCM (1.00 mL) was added HCl/dioxane (4.00 M, 900 uL, 17.7 eq) at 0° C. The mixture was stirred at 25° C. for 1 hrs, concentrated to compound 129 (75.0 mg, 198 umol, 97.3% yield, HCl) as a yellow solid. LC-MS (M+H)⁺: 343.1.

### Preparation of Compound 130

To a solution of compound 129 (83.4 mg. 203 umol, 1.10 eq, Li) and compound 10 (70.0 mg, 185 umol, 1.00 eq, HCl) in DCM (2.00 mL) was added T3P (176 mg, 277 umol, 165 uL, 50.0% purity, 1.50 eq) and DIEA (71.6 mg, 554 umol, 96.6 uL, 3.00 eq). The mixture was stirred at 25° C. for 1.5 hrs, diluted with H₂O (20.0 mL) and extracted with a mixture of dichloromethane and methanol (Dichloromethane: Methanol=10:1, 20.0 mL*3). The combined organic phase was dried over Na₂SO₄ and concentrated to give a residue which was purified by Prep-TLC (Dichloromethane: Methanol=10:1, R_{f}=0.50) to provide compound 130 (80.0 mg, 110 umol, 59.5% yield) as a yellow solid. LC-MS (M+H)⁺: 728.1.

### Example 100: (R)-3-(3-(2,2-dimethylmorpholino)phenyl)-2,2-difluoro-3-((R)-1-(3-(5,6,7,8-tetrahydro-1,8-naphthyridin-2-yl)propyl)piperidine-3-carboxamido)propanoic acid (300)

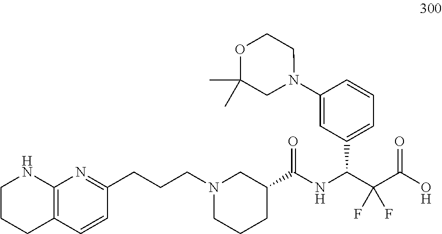 [SMILES: CC1(C)CN(c2cccc([C@@H](NC(=O)[C@@H]3CCCN(CCCc4ccc5c(n4)NCCC5)C3)C(F)(F)C(=O)O)c2)CCO1]

A solution of compound 130 (70.0 mg, 96.2 umol, 1.00 eq) in HCl (4.00 M. 3.50 mL, 146 eq) was stirred at 60° C. for 2 hrs and concentrated to a residue. The residue was purified by Prep-HPLC (column: Waters xbridge 150*25 mm 10 um; mobile phase: [water (10 mM NH₄HCO₃)−ACN]: B %: 24%-44%, 11 min) and Prep-SFC (column: REGIS (S, S) WHELK−O1 (250 mm*25 mm, 10 um): mobile phase: [0.1% NH₃H₂O IPA]: B %: 80%-80%, 4; 30 min) to provide compound 300 (11.88 mg, 19.2 umol, 19.9% yield, 96.9% purity) as an off-white solid. ¹H NMR (400 MHz, CDCl₃) δ 10.5 (s, 1H), 9.47 (d, J=9.2 Hz, 1H), 7.25 (m, 1H), 7.18 (t, J=7.6 Hz, 1H), 7.07-7.04 (m, 2H), 6.77 (d, J=6.8 Hz, 1H), 6.30 (d, J=7.2 Hz, 1H), 5.73-5.66 (m, 1H), 3.81 (t, J=4.8 Hz, 2H), 3.40-3.36 (m, 3H), 3.04-2.88 (m, 6H), 2.71 (d, J=6.0 Hz, 2H), 2.64-2.51 (m, 3H), 2.45-2.39 (m, 1H), 2.22-2.18 (m, 2H), 2.01-1.99 (m, 2H), 1.89-1.86 (m. 3H), 1.62-1.49 (m, 3H), 1.27-1.24 (m, 6H); LC-MS (M+H)⁺: 600.3.

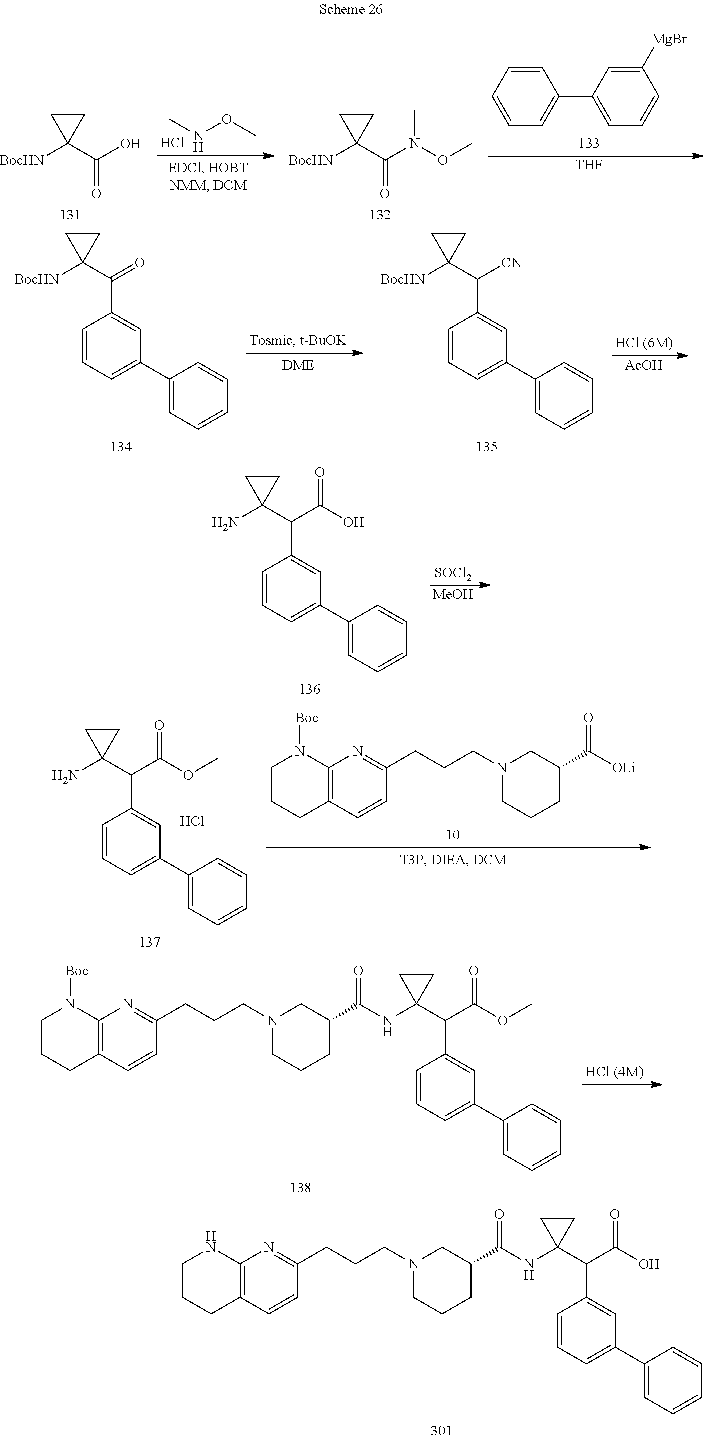

Scheme 26 illustrates the synthesis of compound 301.

### Preparation of Compound 132

To a solution of compound 131 (5.00 g, 24.9 mmol, 1.00 eq) in Dichloromethane (30.0 mL) was added N,O-dimethylhydroxylamine hydrochloride (2.42 g, 24.9 mmol, 1.00 eq), EDCI (7.15 g, 37.3 mmol, 1.50 eq), HOBT (5.04 g, 37.3 mmol, 1.50 eq), NMM (12.6 g, 124 mmol, 13.7 mL, 5.00 eq). The mixture was stirred at 25° C. for 4 hrs, diluted with H₂O (40.0 mL), extracted with Dichloromethane (40.0 mL*3). The combined organic extracts were washed with saturated NaHCO₃ solution (50.0 mL*1), brine (50.0 mL*2), dried over Na₂SO₄, filtered and concentrated to give a residue which was used in the next step directly without any purification. Compound 132 (5.20 g, crude) was obtained as a white solid. LC-MS (M-99)⁺: 145.1.

### Preparation of Compound 134

A solution of compound 132 (3.00 g, 12.3 mmol, 1.00 eq) in THF (40.0 mL) was degassed and purged with N₂ 3 times and compound 133 (0.500 M, 49.1 mL. 2.00 eq) was added dropwise at −40° C. The mixture was stirred at −40° C. for 3 hrs under N₂ atmosphere, quenched by addition of saturated NH₄Cl solution 20.0 mL at 0° C., and then extracted with ethyl acetate 60.0 mL (20.0 mL*3). The combined organic extracts were washed with brine (30.0 mL (15.0 mL*2)), dried over Na₂SO₄, filtered and concentrated to give a residue which were purified by column chromatography (SiO₂, Petroleum ether: Ethyl acetate=1:0 to 0:1) to provide compound 134 (550 mg, 1.63 mmol, 13.3% yield) as a white solid. LC-MS (M-99)⁺: 238.3.

### Preparation of Compound 135

To a solution of compound 134 (550 mg, 1.63 mmol, 1.00 eq) in DME (4.00 mL) was added t-BuOK (183 mg, 1.63 mmol, 1.00 eq) and Tosmic (637 mg, 3.26 mmol, 2.00 eq). The mixture was stirred at 25° C. for 3 hrs, filtered to remove insoluble solid, which was then rinsed with DME (30.0 mL). The combined filtrates were concentrated to give a residue which was purified by Prep-TLC (SiO₂, Petroleum ether: Ethyl acetate=3:1, R_{f}=0.60) to provide compound 135 (80.0 mg, 230 umol, 14.1% yield) as a white solid. LC-MS (M-55)⁺: 293.0.

### Preparation of Compound 136

To a solution of compound 135 (120 mg, 344 umol, 1.00 eq) in AcOH (1.05 g, 17.5 mmol, 1.00 mL, 50.8 eq) was added HCl (6 M, 6.00 mL, 105 eq). The mixture was stirred at 125° C. for 8 hrs, concentrated to give a residue to provide compound 136 (100 mg, crude, HCl) as a yellow oil. LC-MS (M+H)⁺: 268.0.

### Preparation of Compound 137

To a solution of compound 136 (100 mg, 329 umol, 1.00 eq, HCl) in MeOH (2.00 mL) was added SOCl₂ (3.28 g, 27.6 mmol, 2.00 mL, 83.8 eq) at 0° C. The mixture was stirred at 25° C. for 5 hrs was concentrated to give compound 137 (90.0 mg, 283 umol, 86.0% yield, HCl) as a yellow oil.

LC-MS (M+H)⁺: 282.1.

### Preparation of Compound 138

To a solution of compound 137 (120 mg, 292 umol, 1.00 eq, Li) and compound 10 (83.6 mg, 263 umol, 0.900 eq, HCl) in dichloromethane (3.00 mL) was added T3P (372 mg, 585 umol, 348 uL. 50.0% purity, 2.00 eq) and DIEA (151 mg, 1.17 mmol. 204 uL, 4.00 eq) at 0° C. The mixture was stirred at 25° C. for 2 hrs, diluted with saturated NaHCO₃ solution (20.0 mL) and extracted with dichloromethane 30.0 mL (10.0 mL*3). The combined organic extracts was washed with brine 20.0 mL (10.0 mL*2), dried over Na₂SO₄, filtered and concentrated to give a residue which was purified by Prep-TLC (SiO₂, Dichloromethane: Methanol=10:1. R_{f}=0.60) to give compound 138 (60.0 mg, 90.0 umol, 30.8% yield) as a yellow oil. LC-MS (M+H)⁺: 667.6 Example 101: Synthesis of 2-([1,1′-biphenyl]-3-yl)-2-(1-((R)-1-(3-(5,6,7,8-tetrahydro-1,8-naphthyridin-2-yl)propyl)piperidine-3-carboxamido)cyclopropyl)acetic acid (301)

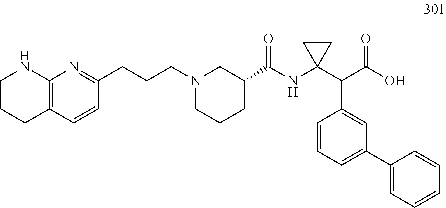 [SMILES: O=C(O)C(c1cccc(-c2ccccc2)c1)C1(NC(=O)[C@@H]2CCCN(CCCc3ccc4c(n3)NCCC4)C2)CC1]

A solution of compound 137 (60.0 mg, 90.0 umol, 1.00 eq) in HCl (4 M, 2.00 mL. 88.9 eq) was stirred at 60° C. for 2 hrs, concentrated to give a residue which was purified by Prep-HPLC (column: Waters xbridge 150*25 mm 10 um; mobile phase: [water (10 mM NH₄HCO₃)−ACN]; B %: 20%-50%, 11 min) to give compound 301 (45.0 mg. 81.4 umol, 90.5% yield) as a yellow oil. LC-MS (M+H)⁺: 553.3.

The stereoisomers of compound 301 were purified by Prep-SFC (column: DAICEL CHIRALPAK AD (250 mm*30 mm, 10 um); mobile phase: [0.1% NH₃H₂O IPA]; B %: 40%-40%, 5.35 min). 301-A (19.74 mg, 35.5 umol, 43.7% yield, 99.5% purity) was obtained as yellow gum. 301-B (22.47 mg, 40.7 umol, 49.9% yield, 100% purity) was obtained as yellow gum.

301-A: ¹H NMR (400 MHz, DMSO-d₆) δ 8.33 (s, 1H), 7.61 (d, J=8.0 Hz, 2H), 7.56-7.50 (m, 2H), 7.47 (t, J=7.6 Hz, 2H), 7.41-7.34 (m, 2H), 7.29 (d, J=7.6 Hz. 1H). 7.09 (d, J=7.2 Hz. 1H), 7.05-6.89 (m, 1H), 6.28 (d, J=7.6 Hz, 1H), 4.08 (s, 1H), 3.51-3.46 (m, 2H), 3.24 (t, J=5.2 Hz. 2H), 2.61 (t, J=6.0 Hz, 2H), 2.46-2.41 (m, 2H), 2.27-2.15 (m, 3H), 2.12-1.96 (m, 2H), 1.78-1.64 (m, 4H), 1.53-1.42 (m, 2H), 1.35-1.31 (m, 1H), 1.23 (s, 1H), 0.84-0.68 (m, 3H), 0.62-0.54 (m, 1H); LC-MS (M+H)⁺: 553.3.

301-B: ¹H NMR (400 MHz, DMSO-d₆) δ 8.28 (s, 1H), 7.61 (d, J=7.6 Hz, 2H), 7.55 (s, 1H), 7.51 (d, J=7.6 Hz, 1H), 7.46 (t, J=8.0 Hz, 2H), 7.40-7.33 (m, 2H), 7.32-7.26 (m, 1H), 7.09 (d, J=7.2 Hz, 1H), 7.03 (br s, 1H), 6.26 (d, J=7.2 Hz, 1H), 4.14 (s, 1H), 3.51-3.45 (m, 2H), 3.24 (s, 3H), 2.61 (t, J=5.6 Hz, 2H), 2.40 (t, J=7.6 Hz, 2H), 2.19-2.11 (m, 2H), 2.09-2.02 (m, 1H), 1.94-1.89 (m, 1H), 1.79-1.71 (m, 2H), 1.66-1.56 (m, 2H), 1.52-1.43 (m, 2H), 1.31-1.24 (m, 2H), 0.80 (s, 3H), 0.63-0.56 (m, 1H); LC-MS (M+H)⁺: 553.3.

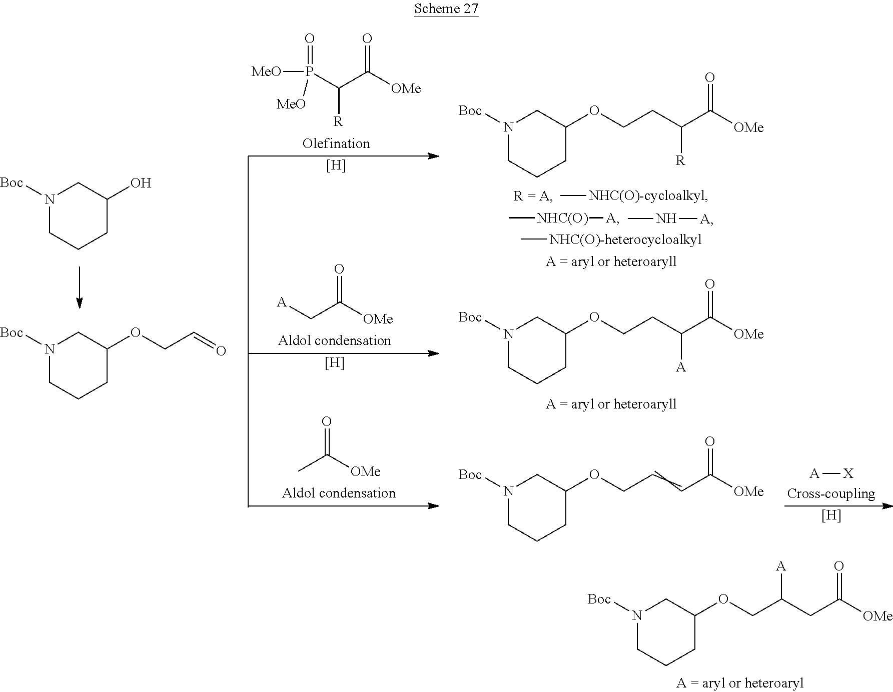

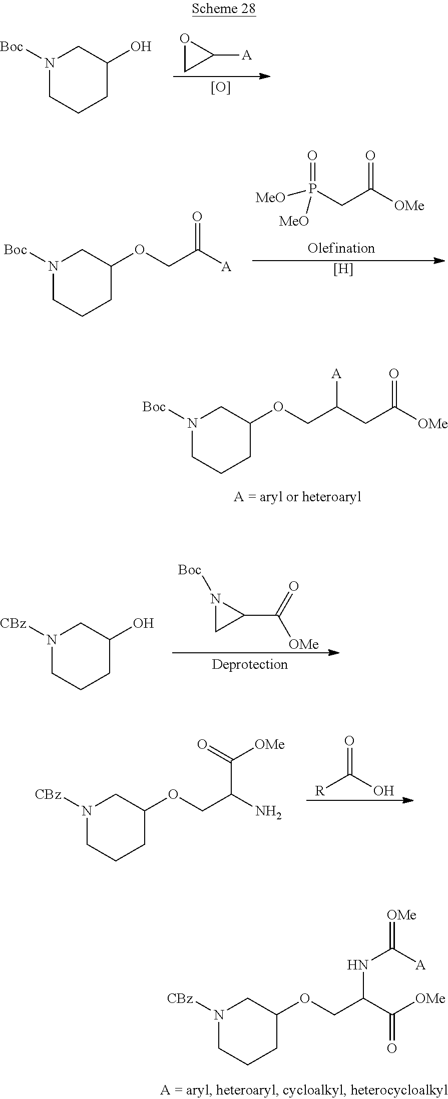

### Example 102: Synthesis of Compound 335

### 1. General Procedure for Preparation of Intermediate 142

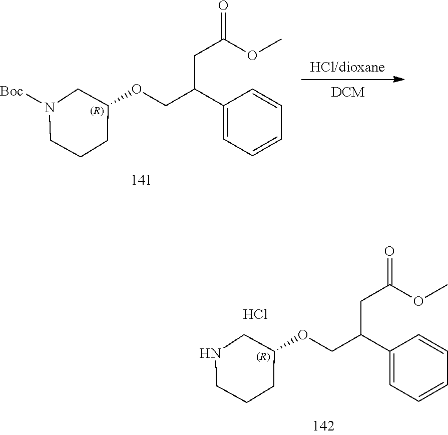

A solution of compound 141 (590 mg, 1.56 mmol, 1.00 eq) in DCM (5.90 mL) was added HCl/dioxane (4.00 M, 5.90 mL, 15.1 eq) at 0° C., the reaction mixture was stirred at 20° C. for 1 hr. LC-MS showed compound 141 was consumed, and one peak with desired mass was detected. The reaction mixture was concentrated under the vacuum to give a residue. Compound 142 (490 mg, 1.56 mmol, 99.9% yield, HCl) was obtained as a yellow oil. LC-MS: (M+H)⁺: 278.2.

### 2. General Procedure for Preparation of Intermediate 144

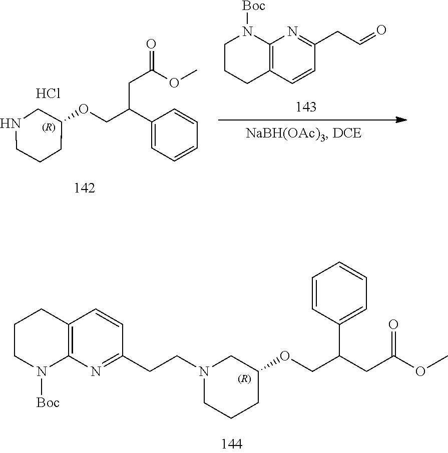

A solution of compound 142 (50.0 mg, 159 umol, 1.00 eq, HCl) and compound 143 (66.0 mg, 239 umol, 1.50 eq) in DCE (1.00 mL) was added NaBH(OAc)₃ (67.5 mg, 319 umol, 2.00 eq), the reaction mixture was stirred at 25° C. for 2 hrs. LC-MS showed compound 142 was consumed, and one peak with desired mass was detected. The reaction mixture was diluted with H₂O 20.0 mL and extracted with a mixture of DCM and MeOH (DCM: MeOH=5:1, 20.0 mL*3), the combined organic phase was dried over Na₂SO₄ and concentrated to give a residue. Compound 144 (70.0 mg, crude) was obtained as a yellow oil. LC-MS: (M+H)⁺: 538.3.

### 3. General Procedure for Preparation of Intermediate 7

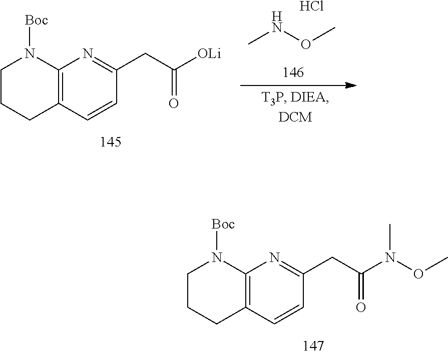

A solution of compound 145 (3M) mg, 1.00 mmol, 1.00 eq, Li) in DCM (4.00 mL) was added compound 146 (147 mg, 1.50 mmol, 1.50 eq), T3P (1.28 g, 2.00 mmol, 1.19 mL, 50.0% purity, 2.00 eq) and DIEA (389 mg, 3.01 mmol, 524 uL, 3.00 eq), the reaction mixture was stirred at 25° C. for 2 hrs. LC-MS showed compound 145 was consumed, and one peak with desired mass was detected. The reaction mixture was diluted with H₂O 20.0 mL and extracted with a mixture of DCM and MeOH (DCM: MeOH=10:1, 20.0 mL*3), the combined organic phase was dried over Na₂SO₄ and concentrated to give a residue. The residue was purified by Prep-TLC (DCM: MeOH=10; 1, R_{f}=0.70). Compound 147 (250 mg. 745 umol, 74.4% yield) was obtained as a yellow oil, confirmed by LC-MS: (M+H)⁺: 336.2.

### 4. General Procedure for Preparation of Intermediate 143

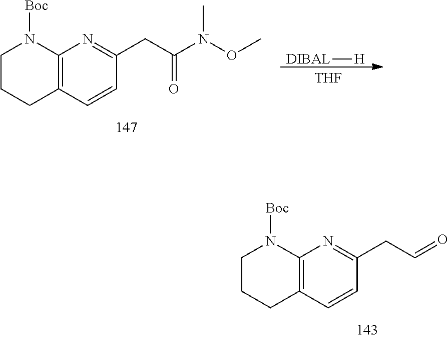

A solution of compound 147 (100 mg, 298 umol, 1.00 eq) in THF (5.00 mL) was added DIBAL-H (1.00 M, 894 uL, 3.00 eq) at −78° C., the reaction mixture was stirred at −78° C. for 2 hrs. TLC (DCM: MeOH=10:1) showed the compound 147 was consumed completely and there was a mainly new spot formed. The reaction mixture was quenched by H₂O 50.0 uL, 15.0% NaOH solution 50.0 uL, and H₂O 150 uL at 0° C., the mixture was stirred at 0° C. for 0.500 hr. Then anhydrous Na₂SO₄ was added after filtered and concentrated under reduced pressure to give a residue. Compound 143 (100 mg, crude) was obtained as a yellow oil.

### 5. General Procedure for Preparation of Compound 335

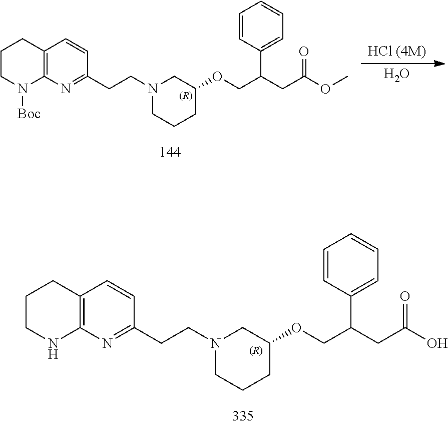

A solution of compound 144 (60.0 mg, 112 umol, 1.00 eq) in HCl (4.00 M, 0.600 mL, 21.5 eq) was stirred at 60° C. for 2 hrs. LC-MS showed compound 144 was consumed, and one peak with desired mass was detected. The reaction mixture was concentrated under the vacuum to give a residue. The residue was purified by Prep-HPLC (column: Waters xbridge 150*25 mm 10 um; mobile phase: [water (10 mM NH₄HCO₃)−ACN]; B %: 12%-42%, 11 min). Compound 335 (14.94 mg, 35.3 umol, 31.6% yield) was obtained as a yellow gum, confirmed by H NMR (400 MHz, DMSO-d₆) δ 7.27-7.19 (m, 5H), 7.02-6.99 (m, 1H), 6.29-6.25 (m, 2H), 3.54-3.50 (m, 3H), 3.22-3.21 (m, 5H), 2.91-2.85 (m, 1H), 2.70-2.54 (m, 6H), 2.45-2.41 (m, 1H), 1.93-1.70 (m, 5H), 1.62-1.57 (m, 1H), 1.39-1.34 (m, 1H), 1.15-1.01 (m, 1H). LC-MS: (M+H)⁺: 424.2.

The following compounds, set forth in Table 7, were prepared according to the general procedures provided in Schemes 1, 22, 27, and 28 or analogous procedures thereto.

TABLE 7

| Compound | |
|---|---|
| No. | ¹H NMR Data |
| 336 | (400 MHz, CDCl₃) δ 9.23 (br s, 1H), 7.30-7.28 (m, 2H), 7.27-7.24 (m, |
| | 2H), 7.19-7.13 (m, 2H), 6.43-6.40 (m, 1H), 3.77-3.59 (m, 2H), 3.53- |
| | 3.35 (m, 6H), 2.87-2.67 (m, 4H), 2.63-2.55 (m, 1H), 2.46-2.27 (m, 3H), |
| | 1.89-1.87 (m, 2H), 1.76-1.68 (m, 2H), 1.45-1.41 (m, 2H). |
| 337 | (400 MHz, DMSO-d₆) δ 10.47-10.15 (m, 1H), 7.31-7.28 (m, 1H), 7.27- |
| | 7.14 (m, 5H), 6.32 (d, J = 7.2 Hz, 1H), 4.14-4.08 (m, 1H), 3.92 (s, 1H), |
| | 3.91-3.87 (m, 1H), 3.75-3.67 (m, 1H), 3.76-3.60 (m, 1H), 3.67-3.51 |
| | (m, 1H), 3.50-3.38 (m, 3H), 3.24-2.91 (m, 4H), 2.79-2.59 (m, 6H), 2.43- |
| | 2.27 (m, 1H), 2.03 (s, 1H), 1.98-1.58 (m, 2H), 1.25 (s, 1H). |
| 353 | (400 MHz, CDCl₃) δ 9.85 (s, 1H), 7.30-7.22 (m, 4H), 7.22-7.11 (m, 2H), |
| | 6.30-6.27 (m, 1H), 3.93-3.79 (m, 1H), 3.63 (dd, J₁ = 10.8 Hz, J; = 5.2 |
| | Hz, 1H), 3.55-3.31 (m, 5H), 3.06-2.96 (m, 1H), 2.88-2.73 (m, 2H), |
| | 2.72-2.48 (m, 6H), 2.19-1.97 (m, 2H), 1.93-1.74 (m, 3H), 1.73-1.62 |
| | (m, 1H), 1.51-1.38 (m, 1H), 1.35-1.18 (m, 1H). |
| 354 | (400 MHz, CDCl₃) δ 9.58 (br s, 1H), 7.31-7.28 (m, 3H), 7.25 (br s, 1H), |
| | 7.23-7.10 (m, 2H), 6.28 (d, J = 7.2 Hz, 1H), 3.89-3.86 (m, 1H), 3.77- |
| | 3.72 (m, 1H), 3.64-3.56 (m, 1H), 3.53-3.36 (m, 4H), 2.98-2.67 (m, 8H), |
| | 2.58 (dd, J₁ = 15.2 Hz, J₂ = 5.2 Hz, 1H), 2.42-2.09 (m, 2H), 1.96-1.82 |
| | (m, 3H), 1.80-1.68 (m, 1H), 1.63-1.49 (m, 1H), 1.43-1.30 (m, 1H). |
| 355 | (400 MHz, CDCl₃) δ 7.28 (s, 2H), 7.27-7.25 (m, 1H), 7.25-7.06 (m, 3H), |
| | 6.40 (d, J = 7.2 Hz, 1H), 3.72-3.65 (m, 2H), 3.56-3.34 (m, 6H), 2.72- |
| | 2.61 (m, 6H), 2.36 (br s, 2H), 1.95-1.75 (m, 3H), 1.65-1.36 (m, 3H). |
| 356 | (400 MHz, CDCl₃) δ 7.31-7.28 (m, 3H), 7.27-7.24 (m, 1H), 7.21-7.15 |
| | (m, 2H), 6.42 (d, J = 7.2 Hz, 1H), 3.84-3.73 (m, 1H), 3.66-3.47 (m, 4H), |
| | 3.46-3.38 (m, 3H), 2.85 (dd, J₁ = 15.2 Hz, J₂ = 9.2 Hz, 1H), 2.75-2.65 |
| | (m, 3H), 2.63-2.48 (m, 2H), 2.47-2.33 (m, 2H), 1.95-1.78 (m, 3H), 1.74- |
| | 1.63 (m, 1H), 1.58-1.42 (m, 2H). |
| 357 | (400 MHz, DMSO-d₆) δ 7.29-7.17 (m, 5H), 7.02 (d, J = 7.2 Hz, 1H), 6.42 |
| | (br s, 1H), 6.24 (d, J = 7.6 Hz, 1H), 3.57-3.47 (m, 2H), 3.32-3.16 (m, |
| | 4H), 2.88 (br d, J = 8.8 Hz, 1H), 2.76-2.54 (m, 4H), 2.48-2.39 (m, 3H), |
| | 2.37-2.27 (m, 2H), 2.03-1.84 (m, 2H), 1.83-1.68 (m, 3H), 1.66-1.48 |
| | (m, 3H), 1.48-1.27 (m, 3H), 1.15-0.99 (m, 1H). |
| 358 | (400 MHz, DMSO-d₆) δ 7.29-7.16 (m, 5H), 7.01 (d, J = 7.2 Hz, 1H), 6.39 |
| | (br s, 1H), 6.24 (d, J = 7.2 Hz, 1H), 3.56-3.48 (m, 2H), 3.27-3.16 (m, |
| | 4H), 2.82 (br d, J = 8.0 Hz, 1H), 2.72-2.66 (m, 1H), 2.59 (br t, J = 6.0 Hz, |
| | 3H), 2.47-2.39 (m, 3H), 2.28-2.21 (m, 2H), 1.89-1.66 (m, 5H), 1.64- |
| | 1.48 (m, 3H), 1.43-1.28 (m, 3H), 1.10-0.96 (m, 1H). |
| 442 | (400 MHz, DMSO-d₆) δ 7.30-7.23 (m, 4H), 7.21-7.15 (m, 1H), 7.05 (d, |
| | J = 7.2 Hz, 1H), 6.80 (s, 1H), 6.28-6.24 (m, 1H), 3.54 (d, J = 3.6 Hz, 2H), |
| | 3.26-3.19 (m, 4H), 2.97 (d, J = 9.2 Hz, 1H), 2.69-2.58 (m, 4H), 2.47- |
| | 2.38 (m, 3H), 2.32-2.20 (m, 2H), 1.89-1.78 (m, 2H), 1.78-1.58 (m, 6H), |
| | 1.40-1.28 (m, 1H), 1.08-1.04 (m, 1H). |
| 445 | (400 MHz, DMSO-d₆) δ 7.47-7.14 (m, 6H), 7.04 (d, J = 7.2 Hz, 1H), 6.28- |
| | 6.21 (m, 1H), 3.23 (s, 1H), 2.64-2.56 (m, 4H), 2.45-2.35 (m, 4H), 2.31- |
| | 2.22 (m, 2H), 1.88-1.56 (m, 9H), 1.39-1.36 (m, 2H), 1.25-1.21 (m, |
| | 2H), 1.04-1.02 (m, 2H). |
| 456 | (400 MHz, DMSO-d₆) δ 7.64 (d, J = 7.6 Hz, 2H), 7.54 (s, 1H), 7.50-7.42 |
| | (m, 3H), 7.40-7.33 (m, 2H), 7.25 (d, J = 7.6 Hz, 1H), 7.04 (d, J = 7.2 Hz, |
| | 1H), 6.74 (s, 1H), 6.25 (d, J = 7.6 Hz, 1H), 3.65-3.60 (m, 2H), 3.32-3.26 |
| | (m, 3H), 3.23-3.22 (m, 3H), 3.01-2.94 (m, 1H), 2.75-2.69 (m, 1H), 2.62- |
| | 2.54 (m, 4H), 2.44-2.37 (m, 2H), 2.34-2.24 (m, 2H), 1.78-1.70 (m, |
| | 4H), 1.64-1.56 (m, 1H), 1.42-1.30 (m, 1H), 1.23 (s, 1H), 1.13-0.99 (m, |
| | 1H). |
| 457 | (400 MHz, DMSO-d₆) δ 7.64 (d, J = 7.2 Hz, 2H), 7.54 (s, 1H), 7.51-7.43 |
| | (m, 3H), 7.40-7.33 (m, 2H), 7.25 (d, J = 7.6 Hz, 1H), 7.03 (d, J = 7.2 Hz, |
| | 1H), 6.68 (s, 1H), 6.25 (d, J = 7.2 Hz, 1H), 3.62 (d, J = 6.8 Hz, 2H), 3.39- |
| | 3.30 (m, 3H), 3.23-3.22 (m, 3H), 2.91 (d, J = 8.4 Hz, 1H), 2.76-2.70 (m, |
| | 1H), 2.64-2.56 (m, 4H), 2.45-2.37 (m, 2H), 2.33-2.23 (m, 2H), 1.88- |
| | 1.80 (m, 2H), 1.76-1.67 (m, 4H), 1.42-1.35 (m, 1H), 1.13-1.03 (m, 1H). |
| 458 | (400 MHz, DMSO-d₆) δ 7.63 (d, J = 7.6 Hz, 1H), 7.57 (d, J = 8.4 Hz, 1H), |
| | 7.45 (t, J = 7.6 Hz, 1H), 7.38-7.30 (m, 3H), 7.05 (d, J = 7.2 Hz, 1H), 6.76 |
| | (s, 1H), 6.27 (d, J = 7.2 Hz, 1H), 3.63-3.56 (m, 3H), 3.24 (t, J = 5.6 Hz, |
| | 1H), 3.01 (d, J = 9.6 Hz, 1H), 2.76-2.64 (m, 2H), 2.60 (t, J = 6.0 Hz, 3H), |
| | 2.47-2.40 (m, 4H), 2.34-2.31 (m, 1H), 2.01-1.90 (m, 1H), 1.85-1.77 |
| | (m, 2H), 1.76-1.70 (m, 3H), 1.66-1.59 (m, 1H), 1.44-1.33 (m, 1H), 1.16- |
| | 1.05 (m, 1H). |
| 459 | (400 MHz, DMSO-d₆) δ 7.63 (d, J = 7.2 Hz, 1H), 7.57 (d, J = 8.4 Hz, 1H), |
| | 7.45 (t, J = 7.6 Hz, 1H), 7.38-7.31 (m, 3H), 7.03 (d, J = 7.2 Hz, 1H), 6.68 |
| | (s, 1H), 6.25 (d, J = 7.2 Hz, 1H), 3.58 (d, J = 6.8 Hz, 3H), 3.23 (t, J = 4.0 |
| | Hz, 1H), 2.90 (d, J = 7.2 Hz, 1H), 2.77-2.64 (m, 2H), 2.59 (t, J = 6.0 Hz, |
| | 3H), 2.44-2.36 (m, 3H), 2.31-2.21 (m, 2H), 1.90-1.80 (m, 2H), 1.77- |
| | 1.67 (m, 4H), 1.65-1.59 (m, 1H), 1.43-1.32 (m, 1H), 1.10-1.03 (m, 1H). |
| 460 | (400 MHz, DMSO-d₆) δ 7.42-7.21 (m, 9H), 7.17-7.05 (m, 2H), 6.27 (d, |
| | J = 7.6 Hz, 1H), 3.51-3.40 (m, 4H), 3.24 (br s, 2H), 3.10-3.04 (m, 1H), |
| | 2.89 (br d, J = 8.8 Hz, 1H), 2.63-2.58 (m, 2H), 2.56-2.54 (m, 1H), 2.45- |
| | 2.35 (m, 2H), 2.31-2.23 (m, 1H), 2.17-2.08 (m, 1H), 1.79-1.69 (m, 4H), |
| | 1.66-1.57 (m, 2H), 1.53-1.46 (m, 1H), 1.39-1.23 (m, 2H), 1.06 (t, J = |
| | 7.2 Hz, 1H), 0.95-0.84 (m, 1H). |
| 461 | (400 MHz, DMSO-d₆) δ 7.43-7.30 (m, 7H), 7.26-7.22 (m, 1H), 7.14- |
| | 7.04 (m, 2H), 6.84 (br s, 1H), 6.25 (d, J = 7.3 Hz, 1H), 3.59-3.46 (m, 2H), |
| | 3.45-3.38 (m, 2H), 3.23 (br s, 2H), 3.05-2.97 (m, 1H), 2.76 (br d, J = 7.8 |
| | Hz, 1H), 2.62-2.58 (m, 2H), 2.56-2.53 (m, 1H), 2.44-2.33 (m, 2H), |
| | 2.27-2.14 (m, 2H), 1.81-1.61 (m, 6H), 1.56-1.47 (m, 2H), 1.29-1.22 |
| | (m, 1H), 1.10-0.83 (m, 2H). |
| 462 | (400 MHz, DMSO-d₆) δ 7.10-7.01 (m, 2H), 6.67 (s, 1H), 6.54-6.45 (m, |
| | 3H), 6.26 (d, J = 7.2 Hz, 1H), 3.98-3.89 (m, 4H), 3.63 (d, J = 11.2 Hz, |
| | 1H), 3.54-3.48 (m, 2H), 3.29 (s, 1H), 3.25-3.22 (m, 3H), 3.14-3.12 (m, |
| | 1H), 2.98-2.92 (m, 1H), 2.69-2.64 (m, 1H), 2.63-2.57 (m, 4H), 2.44- |
| | 2.39 (m, 3H), 2.35-2.25 (m, 3H), 2.04-1.81 (m, 6H), 1.77-1.65 (m, 6H). |
| 463 | (400 MHz, DMSO-d₆) δ 7.09-7.02 (m, 2H), 6.64 (s, 1H), 6.54-6.46 (m, |
| | 3H), 6.25 (d, J = 7.2 Hz, 1H), 3.96-3.91 (m, 4H), 3.65-3.60 (m, 2H), |
| | 3.52-3.50 (m, 2H), 3.24-3.23 (m, 3H), 3.16-3.13 (m, 2H), 2.91-2.86 |
| | (m, 1H), 2.60 (t, J = 6.4 Hz, 4H), 2.43-2.38 (m, 3H), 2.28-2.21 (m, 2H), |
| | 2.03-1.92 (m, 2H), 1.89-1.82 (m, 3H), 1.76-1.71 (m, 2H), 1.69-1.60 |
| | (m, 4H), 1.44-1.34 (m, 1H), 1.11-1.01 (m, 1H). |
| 464 | (400 MHz, DMSO-d₆) δ 7.10-7.02 (m, 2H), 6.59 (s, 1H), 6.50 (t, J = 7.2 |
| | Hz, 3H), 6.27 (d, J = 7.2 Hz, 1H), 3.96-3.91 (m, 4H), 3.62 (d, J = 9.2 Hz, |
| | 2H), 3.53 (s, 2H), 3.26-3.23 (m, 4H), 3.19-3.17 (m, 2H), 3.00-2.96 (m, |
| | 1H), 2.73-2.64 (m, 3H), 2.60 (t, J = 5.2 Hz, 3H), 2.45 (s, 2H), 2.03-1.97 |
| | (m, 2H), 1.94-1.83 (m, 3H), 1.75-1.65 (m. 7H), 1.45 (s, 1H). |
| 465 | (400 MHz, DMSO-d₆) δ 7.08-7.02 (m, 2H), 6.64 (s, 1H), 6.53-6.46 (m, |
| | 3H), 6.25 (d, J = 7.2 Hz, 1H), 3.96-3.90 (m, 4H), 3.63 (d, J = 10.4 Hz, |
| | 1H), 3.55-3.49 (m, 2H), 3.29-3.22 (m, 5H), 3.14-3.10 (m, 1H), 2.94 (d, |
| | J = 9.2 Hz, 1H), 2.68-2.63 (m, 1H), 2.60 (t, J = 6.0 Hz, 4H), 2.45-2.39 |
| | (m, 3H), 2.31-2.24 (m, 2H), 2.02-1.80 (m, 6H), 1.77-1.65 (m, 6H). |
| 481 | (400 MHz, DMSO-d₆) δ 7.08-7.02 (m, 2H), 6.99-6.93 (m, 2H), 6.91- |
| | 6.85 (m, 1H), 6.26 (d, J = 7.2 Hz, 1H), 3.60-3.45 (m, 4H), 3.28-3.21 (m, |
| | 3H), 2.98 (d, J = 9.6 Hz, 1H), 2.66-2.55 (m, 4H), 2.48-2.37 (m, 3H), |
| | 2.35-2.20 (m, 2H), 1.90-1.78 (m, 3H), 1.77-1.71 (m, 3H), 1.70-1.66 |
| | (m, 1H), 1.63-1.55 (m, 1H), 1.42-1.30 (m, 1H), 1.10-0.95 (m, 1H), 0.93- |
| | 0.84 (m, 2H), 0.64-0.55 (m, 2H). |
| 489 | (400 MHz, DMSO-d₆) δ 8.21 (dd, J₁ = 3.6 Hz, J₂ = 2.0 Hz, 2H), 7.29 (t, J = |
| | 1.8 Hz, 1H), 7.06 (d, J = 7.2 Hz, 1H), 6.89 (br s, 1H), 6.26 (d, J = 7.2 |
| | Hz, 1H), 3.57 (dd, J₁ = 6.8 Hz, J₂ = 2.4 Hz, 2H), 3.30-3.14 (m, 4H), 2.96 |
| | (br d, J = 8.8 Hz, 1H), 2.70-2.55 (m, 4H), 2.48-2.35 (m, 3H), 2.35-2.21 |
| | (m, 2H), 1.94-1.83 (m, 2H), 1.82-1.64 (m, 6H), 1.63-1.54 (m, 1H), 1.37- |
| | 1.22 (m, 1H), 1.08-1.00 (m, 1H), 1.00-0.93 (m, 2H), 0.74-0.67 (m, 2H). |
| 490 | (400 MHz, DMSO-d₆) δ 8.25 (d, J = 5.2 Hz, 1H), 7.18 (s, 1H), 7.06 (d, J = |
| | 7.6 Hz, 1H), 7.01 (dd, J₁ = 5.2 Hz, J₂ = 1.2 Hz, 1H), 6.88 (s, 1H), 6.26 (d, J = |
| | 7.2 Hz, 1H), 3.59-3.55 (m, 3H), 3.34-3.31 (m, 2H), 3.25-3.21 (m, |
| | 3H), 2.91 (d, J = 8.0 Hz, 1H), 2.68-2.58 (m, 4H), 2.52 (d, J = 2.0 Hz, 1H), |
| | 2.44-2.39 (m, 2H), 2.33-2.24 (m, 2H), 2.04-1.99 (m, 1H), 1.85-1.81 |
| | (m, 1H), 1.76-1.66 (m, 4H), 1.41-1.33 (m, 1H), 1.27-1.21 (m, 1H), 1.09- |
| | 1.00 (m, 1H), 0.91-0.86 (m, 4H). |
| 491 | (400 MHz, DMSO-d₆ + D₂O) δ 7.20 (t, J = 7.6 Hz, 1H), 7.14 (s, 1H), 7.11- |
| | 7.04 (m, 3H), 6.26 (d, J = 7.2 Hz, 1H), 4.00 (t, J = 8.0 Hz, 1H), 3.95-3.88 |
| | (m, 1H), 3.77 (q, J = 8.0 Hz, 1H), 3.56-3.51 (m, 4H), 3.34-3.29 (m, 1H), |
| | 3.26-3.20 (m, 3H), 3.19-3.14 (m, 1H), 2.94 (d, J = 8.4 Hz, 1H), 2.69- |
| | 2.63 (m, 1H), 2.60 (t, J = 6.0 Hz, 3H), 2.45-2.38 (m, 3H), 2.33-2.24 (m, |
| | 3H), 1.92-1.84 (m, 2H), 1.77-1.72 (m, 4H), 1.63-1.56 (m, 1H), 1.39- |
| | 1.31 (m, 1H), 1.26-1.19 (m, 1H), 1.09-0.98 (m, 1H). |
| 493 | (400 MHz, DMSO-d₆ + D₂O) δ 7.21 (t, J = 7.6 Hz, 1H), 7.15 (s, 1H), 7.11- |
| | 7.03 (m, 3H), 6.26 (d, J = 7.2 Hz, 1H), 4.00 (t, J = 8.0 Hz, 1H), 3.95-3.88 |
| | (m, 1H), 3.77 (q, J = 8.0 Hz, 1H), 3.54-3.49 (m, 4H), 3.36-3.30 (m, 2H), |
| | 3.25-3.19 (m, 3H), 2.89 (d, J = 9.2 Hz, 1H), 2.69-2.64 (m, 1H), 2.59 (t, J = |
| | 6.4 Hz, 3H), 2.46-2.37 (m, 3H), 2.35-2.23 (m, 3H), 1.91-1.86 (m, |
| | 1H), 1.83-1.78 (m, 1H), 1.76-1.67 (m, 4H), 1.65-1.59 (m, 1H), 1.43- |
| | 1.33 (m, 1H), 1.23-1.21 (m, 1H), 1.15-1.03 (m, 1H). |
| 506 | (400 MHz, DMSO-d₆ + D₂O) δ 7.20 (t, J = 7.6 Hz, 1H), 7.13 (s, 1H), 7.11- |
| | 7.04 (m, 3H), 6.27 (d, J = 7.2 Hz, 1H), 4.00 (t, J = 6.8 Hz, 1H), 3.95-3.88 |
| | (m, 1H), 3.77 (q, J = 8.0 Hz, 1H), 3.50-3.47 (m, 2H), 3.37-3.20 (m, 5H), |
| | 3.19-3.12 (m, 1H), 2.94 (d, J = 8.0 Hz, 1H), 2.68-2.62 (m, 1H), 2.61- |
| | 2.54 (m, 4H), 2.45-2.38 (m, 3H), 2.33-2.23 (m, 3H), 1.93-1.84 (m, 2H), |
| | 1.78-1.71 (m, 4H), 1.63-1.57 (m, 1H), 1.39-1.31 (m, 1H), 1.28-1.20 |
| | (m, 1H), 1.10-0.99 (m, 1H). |
| 507 | (400 MHz, DMSO-d₆ + D₂O) δ 7.20 (t, J = 7.6 Hz, 1H), 7.14 (s, 1H), 7.10- |
| | 7.04 (m, 3H), 6.26 (d, J = 7.2 Hz, 1H), 4.00 (t, J = 7.6 Hz, 1H), 3.94-3.88 |
| | (m, 1H), 3.77 (q, J = 8.0 Hz, 1H), 3.53-3.47 (m, 4H), 3.35-3.28 (m, 2H), |
| | 3.25-3.18 (m, 3H), 2.87 (d, J = 10.8 Hz, 1H), 2.68-2.62 (m, 1H), 2.61- |
| | 2.57 (m, 3H), 2.46-2.37 (m, 3H), 2.32-2.21 (m, 3H), 1.90-1.81 (m, 2H), |
| | 1.75-1.67 (m, 4H), 1.63-1.57 (m, 1H), 1.41-1.31 (m, 1H), 1.28-1.18 |
| | (m, 1H), 1.11-1.01 (m, 1H). |
| 508 | (400 MHz, DMSO-d₆) δ 7.08-7.02 (m, 2H), 6.99-6.93 (m, 1H), 6.91- |
| | 6.85 (m, 2H), 6.26 (d, J = 7.2 Hz, 1H), 3.58-3.50 (m, 3H), 3.35-3.27 (m, |
| | 1H), 2.23 (t, J = 5.6 Hz, 2H), 2.89 (d, J = 8.0 Hz, 1H), 2.65-2.55 (m, 4H), |
| | 2.49-2.36 (m, 3H), 2.34-2.18 (m, 2H), 1.90-1.78 (m, 3H), 1.77-1.70 |
| | (m, 3H), 1.69-1.62 (m, 2H), 1.61-1.55 (m, 1H), 1.42-1.31 (m, 1H), 1.09- |
| | 0.97 (m, 1H), 0.94-0.84 (m, 2H), 0.65-0.56 (m, 2H). |
| 513 | (400 MHz, DMSO-d₆) δ 8.21 (br s, 2H), 7.30 (s, 1H), 7.05 (d, J = 7.2 Hz, |
| | 1H), 6.81 (br s, 1H), 6.26 (d, J = 7.2 Hz, 1H), 3.61-3.51 (m, 2H), 3.35- |
| | 3.28 (m, 1H), 3.24-3.23 (m, 3H), 2.88 (br d, J = 8.6 Hz, 1H), 2.67 (dd, J₁ = |
| | 16.0 Hz, J₂ = 6.8 Hz, 1H), 2.60 (br t, J = 6.4 Hz, 3H), 2.48-2.35 (m, |
| | 3H), 2.31-2.23 (m, 2H), 1.93-1.57 (m, 9H), 1.39-1.23 (m, 1H), 1.05- |
| | 1.03 (m, 1H), 1.00-0.94 (m, 2H), 0.76-0.66 (m, 2H). |
| 514 | (400 MHz, DMSO-d₆) δ 8.25 (d, J = 5.2 Hz, 1H), 7.18 (s, 1H), 7.07 (d, J = |
| | 7.2 Hz, 1H), 7.00 (dd, J₁ = 4.8 Hz, J₂ = 1.2 Hz, 2H), 6.27 (d, J = 7.2 Hz, |
| | 1H), 3.58 (d, J = 6.8 Hz, 3H), 3.25-3.22 (m, 3H), 3.15 (t, J = 7.2 Hz, 2H), |
| | 3.01 (d, J = 9.6 Hz, 1H), 2.68-2.63 (m, 1H), 2.61 (t, J = 6.4 Hz, 3H), 2.52 |
| | (d, J = 1.6 Hz, 1H), 2.46-2.42 (m, 2H), 2.34-2.26 (m, 2H), 2.05-1.98 |
| | (m, 1H), 1.92-1.84 (m, 1H), 1.77-1.73 (m, 3H), 1.63-1.57 (m, 1H), 1.40- |
| | 1.32 (m, 1H), 1.25-1.22 (m, 1H), 1.08-0.99 (m, 1H), 0.91-0.86 (m, 4H). |
| 568 | (400 MHz, DMSO-d₆) δ 7.47 (br s, 1H), 7.35 (d, J = 4.8 Hz, 1H), 7.11 (t, |
| | J = 8.0 Hz, 1H), 6.84 (s, 1H), 6.75 (dd, J₁ = 8.0 Hz, J₂ = 2.0 Hz, 1H), 6.69 |
| | (d, J = 7.6 Hz, 1H), 6.48 (d, J = 7.2 Hz, 1H), 3.74-3.71 (m, 3H), 3.61- |
| | 3.57 (m, 3H), 3.34-3.30 (m, 3H), 3.23-3.14 (m, 3H), 3.03-3.00 (m, 4H), |
| | 2.94-2.91 (m, 3H), 2.67-2.61 (m, 5H), 2.07-2.06 (m, 2H), 1.80-1.72 |
| | (m, 5H), 1.45 (br s, 1H), 1.22 (s, 6H). |
| 569 | (400 MHz, DMSO-d₆) δ 7.10 (t, J = 7.6 Hz, 1H), 7.04 (d, J = 7.2 Hz, 1H), |
| | 6.81 (s, 1H), 6.74 (dd, J₁ = 8.4 Hz, J₂ = 2.0 Hz, 1H), 6.67 (d, J = 8.0 Hz, |
| | 2H), 6.25 (d, J = 7.2 Hz, 1H), 3.73 (t, J = 4.8 Hz, 2H), 3.52 (d, J = 6.8 Hz, |
| | 2H), 3.36-3.29 (m, 1H), 3.23-3.22 (m, 3H), 3.01 (t, J = 4.8 Hz, 2H), 2.90- |
| | 2.86 (m, 3H), 2.60-2.58 (m, 4H), 2.42-2.40 (m, 3H), 2.30-2.21 (m, |
| | 2H), 1.75-1.67 (m, 8H), 1.42-1.33 (m, 1H), 1.22 (s, 6H), 1.10-1.01 (m, 1H). |
| 570 | (400 MHz, DMSO-d₆) δ 7.10-7.01 (m, 2H), 6.78 (s, 1H), 6.71 (dd, J₁ = |
| | 8.2 Hz, J₂ = 2.0 Hz, 1H), 6.67 (br s, 1H), 6.61 (d, J = 7.6 Hz, 1H), 6.25 (d, |
| | J = 7.2 Hz, 1H), 3.60-3.46 (m, 3H), 3.30-3.21 (m, 3H), 3.15-3.11 (m, |
| | 1H), 3.03-2.98 (m, 2H), 2.95 (br d, J = 8.8 Hz, 1H), 2.77 (s, 2H), 2.68- |
| | 2.57 (m, 4H), 2.46-2.38 (m, 3H), 2.32-2.26 (m, 2H), 1.86-1.67 (m, 6H), |
| | 1.66-1.58 (m, 3H), 1.39-1.25 (m, 3H), 1.06 (br d, J = 12.6 Hz, 1H), 0.95 |
| | (s, 6H). |
| 571 | (400 MHz, DMSO-d₆) δ 7.11-7.01 (m, 2H), 6.81 (s, 1H), 6.75 (d, J = 8.0 |
| | Hz, 1H), 6.68 (s, 1H), 6.61 (d, J = 7.6 Hz, 1H), 6.26 (d, J = 7.6 Hz, 1H), |
| | 3.55-3.51 (m, 4H), 3.24-3.23 (m, 3H), 3.10 (t, J = 5.2 Hz, 4H), 2.94 (d, J = |
| | 8.4 Hz, 1H), 2.68-2.64 (m, 1H), 2.63-2.57 (m, 4H), 2.44-2.39 (m, |
| | 3H), 2.33-2.25 (m, 2H), 1.91-1.80 (m, 2H), 1.78-1.68 (m, 5H), 1.63- |
| | 1.56 (m, 1H), 1.41 (t, J = 5.6 Hz, 4H), 0.94 (s, 6H). |
| 572 | (400 MHz, DMSO-d₆) δ 7.11-7.01 (m, 2H), 6.78 (s, 1H), 6.74-6.65 (m, |
| | 2H), 6.61 (d, J = 7.6 Hz, 1H), 6.25 (d, J = 7.2 Hz, 1H), 3.52 (br d, J = 6.8 |
| | Hz, 2H), 3.34-3.28 (m, 1H), 3.23-3.20 (m, 2H), 3.19-3.11 (m, 1H), |
| | 3.03-2.98 (m, 2H), 2.90 (br d, J = 9.2 Hz, 1H), 2.77 (s, 2H), 2.68-2.57 |
| | (m, 4H), 2.48-2.33 (m, 4H), 2.32-2.22 (m, 2H), 1.86-1.83 (m, 2H), 1.76- |
| | 1.67 (m, 4H), 1.66-1.59 (m, 3H), 1.46-1.35 (m, 1H), 1.33-1.27 (m, |
| | 2H), 1.12-1.02 (m, 1H), 0.95 (s, 6H). |
| 573 | (400 MHz, DMSO-d₆) δ 7.11-7.02 (m, 2H), 6.81 (s, 1H), 6.77-6.72 (m, |
| | 1H), 6.65 (s, 1H), 6.61 (d, J = 7.6 Hz, 1H), 6.25 (d, J = 7.2 Hz, 1H), 3.52 |
| | (d, J = 6.8 Hz, 4H), 3.23 (s, 3H), 3.10 (t, J = 5.6 Hz, 4H), 2.88 (d, J = 8.4 |
| | Hz, 1H), 2.68-2.64 (m, 1H), 2.60 (t, J = 6.4 Hz, 4H), 2.46-2.39 (m, 3H), |
| | 2.33-2.25 (m, 2H), 1.87-1.80 (m, 2H), 1.76-1.65 (m, 5H), 1.63-1.57 |
| | (m, 1H), 1.41 (t, J = 5.6 Hz, 4H), 0.94 (s, 6H). |
| 574 | (400 MHz, DMSO-d₆) δ 7.42-7.33 (m, 2H), 7.32-7.24 (m, 2H), 7.05 (d, |
| | J = 7.2 Hz, 1H), 6.80 (s, 1H), 6.26 (d, J = 7.2 Hz, 1H), 6.05 (s, 1H), 3.61 |
| | (d, J = 10.4 Hz, 2H), 3.32-3.21 (m, 6H), 2.99 (d, J = 8.8 Hz, 1H), 2.73- |
| | 2.67 (m, 1H), 2.60 (t, J = 6.0 Hz, 3H), 2.52 (d, J = 1.6 Hz, 1H), 2.48-2.37 |
| | (m, 3H), 2.35-2.31 (m, 1H), 2.26 (s, 3H), 2.17 (s, 3H), 1.95-1.85 (m, |
| | 1H), 1.78-1.72 (m, 4H), 1.62-1.57 (m, 1H), 1.40-1.31 (m, 1H), 1.11- |
| | 1.00 (m, 1H). |
| 575 | (400 MHz, DMSO-d₆) δ 7.43-7.36 (m, 2H), 7.34-7.27 (m, 2H), 7.24- |
| | 7.09 (m, 1H), 6.35 (d, J = 7.6 Hz, 1H), 6.06 (s, 1H), 3.72 (s, 1H), 3.68- |
| | 3.58 (m, 3H), 3.38-3.31 (m, 2H), 3.28-3.22 (m, 3H), 3.00 (s, 2H), 2.82 |
| | (dd, J₁ = 16.0 Hz, J₂ = 5.6 Hz, 1H), 2.65-2.61 (m, 2H), 2.60-2.54 (m, |
| | 2H), 2.52 (d, J = 1.6 Hz, 2H), 2.27 (s, 3H), 2.17 (s, 3H), 1.97 (d, J = 7.2 |
| | Hz, 1H), 1.79-1.72 (m, 3H), 1.70-1.61 (m, 1H), 1.55 (s, 1H), 1.35-1.07 |
| | (m, 2H). |
| 576 | (400 MHz, DMSO-d₆) δ 7.49-7.37 (m, 4H), 7.05 (d, J = 7.2 Hz, 1H), 6.83 |
| | (s, 1H), 6.75 (s, 1H), 6.26 (d, J = 7.2 Hz, 1H), 3.62 (d, J = 6.8 Hz, 2H), |
| | 3.34-3.29 (m, 4H), 3.23 (s, 3H), 2.98 (d, J = 9.6 Hz, 1H), 2.74-2.67 (m, |
| | 1H), 2.62-2.53 (m, 4H), 2.46-2.36 (m, 3H), 2.32 (s, 3H), 1.95-1.84 (m, |
| | 1H), 1.78-1.72 (m, 4H), 1.63-1.55 (m, 1H), 1.43-1.28 (m, 1H), 1.12- |
| | 1.00 (m, 1H). |
| 577 | (400 MHz, DMSO-d₆) δ 7.50-7.37 (m, 4H), 7.05 (d, J = 7.2 Hz, 1H), 6.75 |
| | (s, 1H), 6.71 (s, 1H), 6.25 (d, J = 7.2 Hz, 1H), 3.63-3.60 (m, 2H), 3.39- |
| | 3.33 (m, 4H), 3.23 (s, 3H), 2.93 (d, J = 6.8 Hz, 1H), 2.77-2.69 (m, 1H), |
| | 2.63-2.54 (m, 4H), 2.45-2.37 (m, 3H), 2.32 (s, 3H), 1.84-1.79 (m, 1H), |
| | 1.77-1.69 (m, 4H), 1.66-1.60 (m, 1H), 1.45-1.33 (m, 1H), 1.19-1.03 |
| | (m, 1H) 8 7.64 (d, J = 7.2 Hz, 2H), 7.54 (s, 1H), 7.51-7.43 (m, 3H), 7.40- |
| | 7.33 (m, 2H), 7.25 (d, J = 7.6 Hz, 1H), 7.03 (d, J = 7.2 Hz, 1H), 6.68 (s, |
| | 1H), 6.25 (d, J = 7.2 Hz, 1H), 3.62 (d, J = 6.8 Hz, 2H), 3.39-3.30 (m, 3H), |
| | 3.23-3.22 (m, 3H), 2.91 (d, J = 8.4 Hz, 1H), 2.76-2.70 (m, 1H), 2.64- |
| | 2.56 (m, 4H), 2.45-2.37 (m, 2H), 2.33-2.23 (m, 2H), 1.88-1.80 (m, 2H), |
| | 1.76-1.67 (m, 4H), 1.42-1.35 (m, 1H), 1.13-1.03 (m, 1H). |
| 579 | (400 MHz, DMSO-d₆) δ 7.04 (d, J = 8.0 Hz, 1H), 6.68 (s, 1H), 6.58 (d, J = |
| | 8.0 Hz, 2H), 6.49 (s, 1H), 6.25 (d, J = 4.0 Hz, 1H), 3.72 (t, J = 4.0 Hz, 2H), |
| | 3.56-3.45 (m, 3H), 3.32 (s, 2H), 3.23 (d, J = 4.0 Hz, 2H), 3.10 (t, J = 4.0 |
| | Hz, 1H), 3.00-2.93 (m, 3H), 2.88 (s, 2H), 2.67-2.64 (m, 2H), 2.59 (t, J = |
| | 8.0 Hz, 4H), 2.33-2.29 (m, 3H), 2.20 (s, 3H), 1.84-1.79 (m, 2H), 1.75- |
| | 1.58 (m, 6H), 1.22 (s, 6H). |
| 581 | (400 MHz, DMSO-d₆) δ 7.04 (d, J = 8.0 Hz, 1H), 6.66 (s, 1H), 6.58 (d, J = |
| | 16 Hz, 2H), 6.49 (s, 1H), 6.25 (s, J = 8.0 Hz, 1H), 3.72 (t, J = 4.0 Hz, 3H), |
| | 3.50 (d, J = 8.0 Hz, 3H), 3.33-3.31 (m, 2H), 3.23 (s, 2H), 3.13 (q, J = 8.0 |
| | Hz, 1H), 2.99 (t, J = 4.0 Hz, 2H), 2.90-2.88 (m, 3H), 2.66 (d, J = 4.0 Hz, |
| | 1H), 2.60 (q, J = 4.0 Hz, 4H), 2.41 (q, J = 8.0 Hz, 3H), 2.20 (s, 3H), 1.84 |
| | (d, J = 8.0 Hz, 2H), 1.77-1.59 (m, 6H), 1.22 (s, 6H). |
| 647 | (400 MHz, DMSO-d6) δ 7.30 (d, J = 8.0 Hz, 1H), 7.07 (br d, J = 7.2 Hz, |
| | 1H), 7.02 (s, 1H), 6.95 (br d, J = 8.4 Hz, 1H), 6.63 (br s, 1H), 6.28 (br d, J = |
| | 7.2 Hz, 1H), 3.75 (br s, 2H), 3.62-3.53 (m, 3H), 3.46 (s, 1H), 3.30- |
| | 3.17 (m, 4H), 3.08 (br d, J = 10.4 Hz, 1H), 2.88 (s, 2H), 2.74-2.70 (m, |
| | 4H), 2.67 (br d, J = 6.4 Hz, 1H), 2.61 (br t, J = 5.6 Hz, 3H), 2.48-2.40 (m, |
| | 3H), 1.93-1.63 (m, 7H), 1.48 (br s, 1H), 1.26 (s, 6H). |
| 648 | (400 MHz, DMSO-d6) δ 7.29 (d, J = 8.4 Hz, 1H), 7.07-7.00 (m, 2H), 6.93 |
| | (br d, J = 8.0 Hz, 1H), 6.73 (br s, 1H), 6.25 (d, J = 7.2 Hz, 1H), 3.75 (br s, |
| | 2H), 3.58-3.52 (m, 2H), 3.33-3.28 (m, 1H), 3.25-3.20 (m, 3H), 2.88 (br |
| | s, 3H), 2.74 (s, 2H), 2.68-2.56 (m, 4H), 2.47-2.37 (m, 3H), 2.33-2.19 |
| | (m, 2H), 1.91-1.81 (m, 2H), 1.77-1.56 (m, 6H), 1.44-1.34 (m, 1H), 1.26 |
| | (s, 6H), 1.12-0.99 (m, 1H). |
| 649 | (400 MHz, DMSO-d6) δ 8.18 (d, J = 2.4 Hz, 1H), 7.92 (s, 1H), 7.31 (br s, |
| | 1H), 7.06 (d, J = 7.2 Hz, 1H), 6.71 (br s, 1H), 6.27 (d, J = 7.4 Hz, 1H), 3.64- |
| | 3.52 (m, 3H), 3.39-3.35 (m, 4H), 3.25-3.21 (m, 3H), 2.93 (br d, J = 9.2 |
| | Hz, 1H), 2.73-2.67 (m, 2H), 2.60 (br t, J = 6.0 Hz, 2H), 2.54 (s, 1H), 2.48- |
| | 2.28 (m, 4H), 2.11-1.95 (m, 5H), 1.86-1.57 (m, 6H), 1.51-1.32 (m, |
| | 1H), 1.23 (s, 1H), 1.19-1.06 (m, 1H). |
| 650 | (400 MHz, DMSO-d6) δ 7.05 (d, J = 7.2 Hz, 1H), 6.77 (d, J = 6.4 Hz, 2H), |
| | 6.69 (s, 1H), 6.26 (d, J = 7.2 Hz, 1H), 3.71 (s, 2H), 3.53 (d, J = 6.4 Hz, |
| | 2H), 3.25 (s, 2H), 3.25-3.10 (m, 2H), 3.05 (s, 2H), 2.96 (s, 2H), 2.90- |
| | 2.85 (m, 1H), 2.66 (s, 1H), 2.61-2.58 (m, 3H), 2.45-2.42 (m, 3H), 2.42- |
| | 2.41 (m, 2H), 1.84-1.81 (m, 2H), 1.75-1.68 (m, 6H), 1.37-1.36 (m, 2H), |
| | 1.20 (s, 6H). |
| 651 | (400 MHz, DMSO-d6) δ 7.13-7.08 (m, 1H), 7.05 (d, J = 7.2 Hz, 1H), 6.85 |
| | (s, 1H), 6.79 (d, J = 8.4 Hz, 1H), 6.70-6.65 (m, 2H), 6.26 (d, J = 7.2 Hz, |
| | 1H), 3.57-3.48 (m, 3H), 3.35 (s, 2H), 3.23 (t, J = 5.6 Hz, 4H), 3.18 (d, J = |
| | 6.8 Hz, 1H), 3.01 (s, 2H), 2.92 (d, J = 9.2 Hz, 1H), 2.70-2.63 (m, 2H), |
| | 2.60 (t, J = 6.0 Hz, 2H), 2.48-2.40 (m, 3H), 2.38-2.28 (m, 2H), 2.14- |
| | 2.05 (m, 2H), 2.01-1.94 (m, 1H), 1.86-1.78 (m, 2H), 1.76-1.72 (m, 2H), |
| | 1.65-1.59 (m, 1H), 1.43-1.36 (m, 1H), 1.23 (s, 1H), 1.07 (s, 6H). |

### Example 102: Synthesis of Compound 382

### 1. General Procedure for Preparation of Intermediate 153

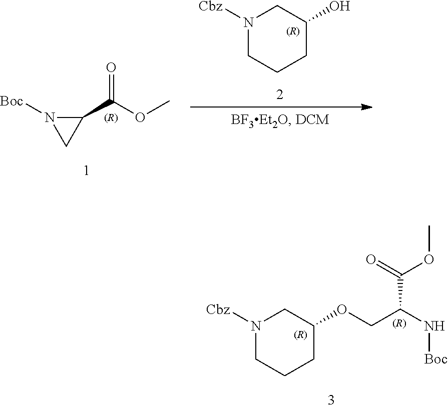

To a solution of compound 151 (0.500 g, 2.48 mmol, 1.00 eq) in DCM (5.00 mL) was added compound 152 (1.17 g, 4.97 mmol, 2.00 eq) and BF₃⋅Et₂O (35.2 mg, 248 umol, 30.6 uL, 0.100 eq) at 0° C. The mixture was stirred at 25° C. for 2 hrs. LC-MS showed˜35.9% of desired mass was detected. The reaction mixture was diluted with DCM (20.0 mL) and washed with sat·aq NaHCO₃(15.0 mL*2), dried over Na₂SO₄, filtered and concentrated under reduced pressure to give a residue. The residue was purified by Prep-TLC (SiO₂, Petroleum ether EtOAc=1:1). Compound 153 (0.300 g, 687 umol, 27.6% yield) was obtained as a light yellow oil and confirmed by ¹H NMR (400 MHz, CDCl₃) δ 7.43-7.26 (m, 5H), 5.07 (s, 2H), 4.19 (q, J=6.4 Hz, 1H), 3.70-3.40 (m, 10H), 1.86-1.72 (m, 1H), 1.67-1.57 (m, 1H), 1.55-1.43 (m, 2H), 1.37 (s, 9H).

### 2. General Procedure for Preparation of Intermediate 154

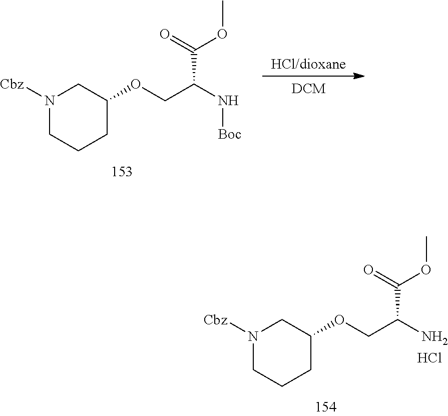 [SMILES: COC(=O)[C@@H](CO[C@@H]1CCCN(C(=O)OCc2ccccc2)C1)NC(=O)OC(C)(C)C ; COC(=O)[C@H](N)CO[C@@H]1CCCN(C(=O)OCc2ccccc2)C1 ; Cl]

To a solution of compound 153 (0.250 g, 572 umol, 1.00 eq) in DCM (2.00 mL) was added HC/dioxane (4.00 M, 2.86 mL, 20.0 eq) at 0° C. The mixture was stirred at 25° C. for 2 hrs. LC-MS showed compound 153 was consumed completely and one main peak with desired mass was detected. The reaction mixture was concentrated under reduced pressure to give a residue. Compound 154 (0.200 g, crude, HCl) was obtained as a yellow oil. LC-MS: (M+H)⁺: 337.2

### 3. General procedure for preparation of intermediate 156

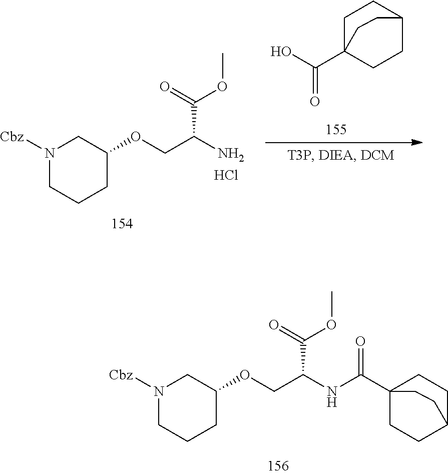 [SMILES: COC(=O)[C@H](N)CO[C@@H]1CCCN(C(=O)OCc2ccccc2)C1 ; Cl ; O=C(O)C12CCC(CC1)CC2 ; COC(=O)[C@@H](CO[C@@H]1CCCN(C(=O)OCc2ccccc2)C1)NC(=O)C12CCC(CC1)CC2]

A mixture of compound 154 (0.200 g, 536 umol, 1.00 eq, HCl), compound 5 (99.2 mg. 643 umol, 1.20 eq), T3P (682 mg, 1.07 mmol, 638 uL, 50.0% purity, 2.00 eq), DIEA (138 mg, 1.07 mmol, 186 uL, 2.00 eq) in DCM (2.00 mL) was degassed and purged with N₂ for 3 times, and then the mixture was stirred at 25° C. for 8 hrs under N₂ atmosphere. LC-MS showed compound 154 was consumed completely and one main peak with desired mass was detected. The reaction mixture was diluted with water (10.0 mL) and extracted with DCM (8.00 mL*3). The combined organic layers were washed with brine (15.0 mL), dried over Na₂SO₄, filtered and concentrated under reduced pressure to give a residue. The residue was purified by Prep-TLC (SiO₂, Petroleum ether: EtOAc=1:1). Compound 156 (150 mg, crude) was obtained as a light yellow oil and confirmed by LC-MS (M+H)⁺: 473.2.

### 4. General Procedure for Preparation of Intermediate 157

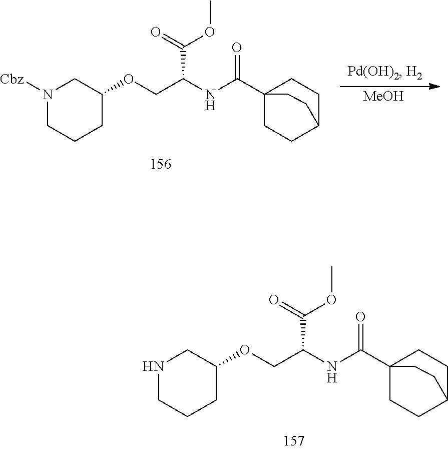 [SMILES: "MeOH" ; COC(=O)[C@@H](CO[C@@H]1CCCN(C(=O)OCc2ccccc2)C1)NC(=O)C12CCC(CC1)CC2 ; COC(=O)[C@@H](CO[C@@H]1CCCNC1)NC(=O)C12CCC(CC1)CC2]

To a solution of compound 156 (0.130 g, 275 umol, 1.00 eq) in MeOH (2.00 mL) was added Pd(OH)₂ (20%. 10 mg) under N₂ atmosphere. The suspension was degassed and purged with H₂ for 3 times. The mixture was stirred under H₂ (15 Psi) at 25° C. for 4 hrs. LC-MS showed compound 156 was consumed completely and one main peak with desired mass was detected. The reaction mixture was concentrated under reduced pressure to give a residue. Compound 157 (80.0 mg, crude) was obtained as a light yellow oil and confirmed by H NMR (400 MHz, CDCl₃) δ 6.38 (d, J=8.0 Hz, 1H), 4.71-4.65 (m, 1H), 3.90 (dd, J₁=9.2 Hz, J₂=2.8 Hz, 1H), 3.74 (s, 3H), 3.67 (dd, J₁=9.2 Hz, J₂=3.2 Hz, 1H), 3.36-3.31 (m, 1H), 2.91-2.86 (m, 1H), 2.80-2.72 (m, 1H), 1.98-1.85 (m, 2H), 1.77-1.68 (m, 8H), 1.66-1.58 (m, 7H), 1.49-1.40 (m, 1H), 1.27-1.16 (m, 1H).

### 5. General procedure for preparation of intermediate 159

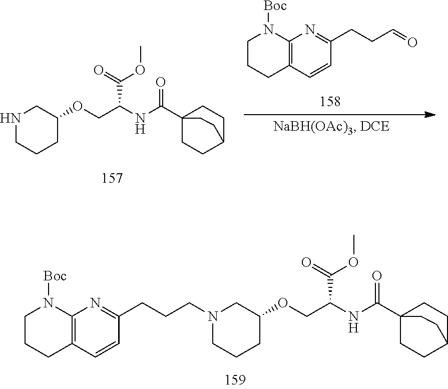 [SMILES: COC(=O)[C@@H](CO[C@@H]1CCCNC1)NC(=O)C12CCC(CC1)CC2 ; CC(C)(C)OC(=O)N1CCCc2ccc(CCC=O)nc21 ; COC(=O)[C@@H](CO[C@@H]1CCCN(CCCc2ccc3c(n2)N(C(=O)OC(C)(C)C)CCC3)C1)NC(=O)C12CCC(CC1)CC2]

A mixture of compound 157 (70.0 mg, 206 umol, 1.00 eq), compound 158 (120 mg, 413 umol, 2.00 eq). NaBH(OAc)₃ (65.7 mg, 310 umol, 1.50 eq) in DCE (1.00 mL) was degassed and purged with N₂ for 3 times, and then the mixture was stirred at 25° C. for 2 hrs under N₂ atmosphere. LC-MS showed 21.9% of desired mass was detected. The reaction mixture was concentrated under reduced pressure to give a residue. The residue was purified by prep-TLC (SiO₂, DCM: MeOH=10:1). Compound 159 (30.0 mg, 48.9 umol, 23.6% yield) was obtained as a yellow oil and confirmed by LC-MS (M+H)⁺: 613.5.

### 6. General Procedure for Preparation of Compound 382

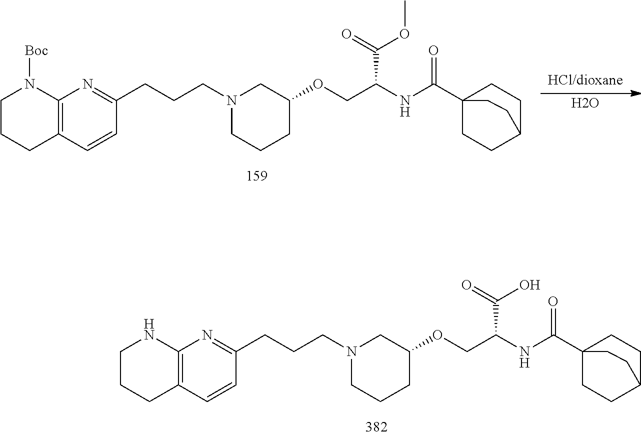 [SMILES: COC(=O)[C@@H](CO[C@@H]1CCCN(CCCc2ccc3c(n2)N(C(=O)OC(C)(C)C)CCC3)C1)NC(=O)C12CCC(CC1)CC2 ; O ; O=C(O)[C@@H](CO[C@@H]1CCCN(CCCc2ccc3c(n2)NCCC3)C1)NC(=O)C12CCC(CC1)CC2]

To a solution of compound 159 (25.0 mg. 40.8 umol, 1.00 eq) in H₂O (0.500 mL) was added HCl/dioxane (4.00 M, 509 uL, 50.0 eq). The mixture was stirred at 60° C. for 2 hrs. LC-MS showed compound 159 was consumed completely and one main peak with desired mass was detected. The reaction mixture was concentrated under reduced pressure to give a residue. The residue was purified by Prep-HPLC (neutral condition; column: Waters xbridge 150*25 mm 10 um; mobile phase: [water (NH₄HCO₃)−ACN]; B %: 15%-45%, 11 min). Compound 382 (11.38 mg, 22.2 umol, 54.4% yield, 97.3% purity) was obtained as an off-white solid and confirmed by ¹H NMR (400 MHz, DMSO-d₆) δ 7.04 (dd. J₁=22.0 Hz, J₂=7.2 Hz, 2H), 6.64 (br s, 1H), 6.28 (d, J=7.2 Hz, 1H), 4.21-4.11 (m, 1H), 3.41 (br s, 1H), 3.24 (s, 2H), 2.86 (d, J=10.0 Hz, 1H), 2.70-2.52 (m, 4H), 2.48-2.38 (m, 5H), 2.24 (br s, 2H), 1.83-1.66 (m, 6H), 1.64-1.47 (m, 13H), 1.44-1.35 (m, 1H), 1.27-1.17 (m, 1H).

The following compounds, set forth in Table 8, were prepared according to the general procedures provided in Schemes 1, 22, 27, and 28 or analogous procedures thereto.

TABLE 8

| Compound | |
|---|---|
| No. | ¹H NMR Data |
| 331 | (400 MHz, CDCl₃) δ 10.14 (br s, 1H), 7.23 (d, J = 7.2 Hz, 1H), 6.66 (d, J = |
| | 6.0 Hz, 1H), 6.30 (d, J = 6.0 Hz, 1H), 4.43-4.41 (m, 1H), 3.80-3.63 |
| | (m, 3H), 3.50-3.47 (m, 3H), 2.92-2.85 (m, 1H), 2.72 (d, J = 6.4 Hz, |
| | 4H), 2.61-2.43 (m, 2H), 2.10-2.01 (m, 4H), 1.93-1.90 (m, 4H), 1.77- |
| | 1.73 (m, 8H), 1.61-1.57 (m, 8H), 1.42-1.30 (m, 1H). |
| 332 | (400 MHz, CDCl₃) δ 10.40 (br s, 1H), 8.62 (s, 1H), 7.94 (d, J = 8.4 Hz, |
| | 1H), 7.78 (d, J = 8.4 Hz, 1H), 7.70-7.68 (m, 2H), 7.41 (t, J = 7.2 Hz, |
| | 1H), 7.21 (d, J = 7.2 Hz, 1H), 6.24 (d, J = 7.6 Hz, 1H), 4.96-4.92 (m, |
| | 1H), 3.95-3.93 (m, 1H), 3.82-3.78 (m, 1H), 3.51-3.46 (m, 3H), 3.08 |
| | (br s, 1H), 2.73-2.68 (m, 6H), 2.39-2.34 (m, 5H), 1.91-1.88 (m, 4H), |
| | 1.79-1.70 (m, 2H), 1.59-1.58 (m, 2H). |
| 333 | (400 MHz, CDCl₃) δ 10.34 (br s, 1H), 7.22 (d, J = 7.2 Hz, 1H), 6.98 (d, J = |
| | 6.8 Hz, 1H), 6.28 (d, J = 7.2 Hz, 1H), 4.46-4.42 (m, 1H), 3.73-3.70 |
| | (m, 2H), 3.47-3.44 (m, 3H), 3.21-3.19 (m, 1H), 2.75-2.69 (m, 5H), |
| | 2.58 (br s, 1H), 2.35 (br s, 2H), 2.19-2.18 (m, 2H), 2.12-1.98 (m, 2H), |
| | 1.88-1.85 (m, 3H), 1.79-1.75 (m, 8H), 1.61-1.58 (m, 8H), 1.49-1.43 |
| | (m, 1H). |
| 334 | (400 MHz, CDCl₃) δ 10.21 (br s, 1H), 8.62 (s, 1H), 7.91 (d, J = 8.4 Hz, |
| | 1H), 7.71-7.60 (m, 2H), 7.25 (d, J = 7.2 Hz, 1H), 6.32 (d, J = 7.2 Hz, |
| | 1H), 4.92-4.88 (m, 1H), 3.93-3.90 (m, 1H), 3.79-3.76 (m, 2H), 3.44 (s, |
| | 3H), 2.90-2.87 (m, 1H), 2.73-2.70 (m, 5H), 2.54 (br s, 1H), 2.37-2.33 |
| | (m, 2H), 2.18-2.02 (m, 3H), 1.91-1.88 (m, 3H), 1.77-1.70 (m, 2H), |
| | 1.45-1.42 (m, 2H). |
| 381 | (400 MHz, CDCl₃) δ 10.24 (s, 1H), 7.22 (d, J = 6.8 Hz, 1H), 6.98 (d, J = |
| | 5.2 Hz, 1H), 6.41 (d, J = 6.8 Hz, 1H), 4.38-4.33 (m, 1H), 3.76 (d, J = |
| | 13.6 Hz, 1H), 3.62-3.57 (m, 1H), 3.51-3.41 (m, 4H), 3.35 (d, J = 13.6 |
| | Hz, 1H), 2.74 (t, J = 6.0 Hz, 2H), 2.62-2.57 (m, 2H), 2.27-2.22 (m, 2H), |
| | 1.95-1.89 (m, 3H), 1.77-1.73 (m, 8H), 1.62-1.58 (m, 8H), 1.42-1.39 |
| | (m, 2H). |
| 383 | (400 MHz, CDCl₃) δ 7.24 (d, J = 7.2 Hz, 1H), 6.87 (d, J = 6.4 Hz, 1H), |
| | 6.37 (d, J = 7.2 Hz, 1H), 4.45-4.40 (m, 1H), 3.77-3.70 (m, 1H), 3.60- |
| | 3.53 (m, 1H), 3.47 (t, J = 5.6 Hz, 3H), 3.20 (d, J = 11.4 Hz, 1H), 3.00- |
| | 2.94 (m, 2H), 2.73 (t, J = 6.0 Hz, 3H), 2.33-2.23 (m, 2H), 2.19-2.09 (m, |
| | 2H), 1.93-1.89 (m, 2H), 1.78-1.73 (m, 8H), 1.64-1.57 (m, 10H), 0.88- |
| | 0.83 (m, 1H). |
| 384 | (400 MHz, CDCl₃) δ 10.85 (s, 1H), 7.20 (d, J = 7.2 Hz, 1H), 6.99 (d, J = |
| | 7.6 Hz, 1H), 6.23 (d, J = 6.8 Hz, 1H), 4.46 (s, 1H), 3.79-3.71 (m, 2H), |
| | 3.52-3.40 (m, 4H), 2.89-2.83 (m, 1H), 2.75-2.67 (m, 3H), 2.66-2.58 |
| | (m, 3H), 2.52-2.46 (m, 1H), 2.22-2.15 (m, 1H), 2.05-1.96 (m, 1H), |
| | 1.95-1.86 (m, 3H), 1.84-1.65 (m, 12H), 1.64-1.57 (m, 8H), 1.54-1.52 |
| | (m, 1H). |
| 385 | (400 MHz, CDCl₃) δ 10.83 (br s, 1H), 8.59 (s, 1H), 8.02 (br d, J = 8.4 Hz, |
| | 1H), 7.80-7.75 (m, 1H), 7.70-7.65 (m, 2H), 7.43-7.38 (m, 1H), 7.17 |
| | (br d, J = 7.2 Hz, 1H), 6.17 (d, J = 7.2 Hz, 1H), 5.07-5.00 (m, 1H), 3.93- |
| | 3.84 (m, 2H), 3.52-3.45 (m, 3H), 2.69 (br t, J = 5.6 Hz, 3H), 2.57-2.32 |
| | (m, 8H), 2.31-2.17 (m, 1H), 1.96-1.86 (m, 3H), 1.78-1.61 (m, 3H), |
| | 1.57-1.45 (m, 3H), 1.43-1.31 (m, 1H). |
| 386 | (400 MHz, CDCl₃) δ 9.28 (br s, 1H), 8.59 (s, 1H), 7.94 (d, J = 8.4 Hz, |
| | 1H), 7.73 (d, J = 8.4 Hz, 1H), 7.63 (t, J = 6.8 Hz, 2H), 7.33 (t, J = 7.2 Hz, |
| | 1H), 7.16 (d, J = 7.2 Hz, 1H), 6.29 (d, J = 7.2 Hz, 1H), 4.93 (q, J = 5.6 |
| | Hz, 1H), 3.84-3.67 (m, 2H), 3.51 (br s, 1H), 3.46-3.36 (m, 2H), 3.05- |
| | 2.87 (m, 2H), 2.86-2.77 (m, 3H), 2.71-2.61 (m, 2H), 2.58-2.30 (m, |
| | 5H), 1.92-1.65 (m, 4H), 1.55-1.37 (m, 2H). |
| 387 | (400 MHz, DMSO-d₆) δ 8.45 (s, 1H), 8.36 (d, J = 8.0 Hz, 1H), 8.23 (d, J = |
| | 7.6 Hz, 1H), 7.85-7.74 (m, 1H), 7.74-7.69 (m, 1H), 7.55 (t, J = 7.6 |
| | Hz, 1H), 7.04 (d, J = 7.2 Hz, 1H), 6.39 (d, J = 7.2 Hz, 1H), 6.34 (s, 1H), |
| | 4.82 (m, 1H), 3.60-3.53 (m, 2H), 3.31-3.28 (m, 4H), 3.22 (s, 2H), 2.86 |
| | (d, J = 8.8 Hz, 1H), 2.62-2.57 (m, 2H), 2.17-2.05 (m, 2H), 2.00-1.90 |
| | (m, 2H), 1.76-1.72 (m, 3H), 1.71-1.55 (m, 1H), 1.38-1.32 (m, 1H), |
| | 1.23-1.06 (m, 1H). |
| 388 | (400 MHz, DMSO-d₆) δ 8.41-8.20 (m, 2H), 7.54-7.09 (m, 2H), 6.95 (d, |
| | J = 6.4 Hz, 1H), 6.36 (s, 1H), 6.14 (d, J = 6.0 Hz, 1H), 5.23 (s, 1H), 4.15 |
| | (s, 1H), 3.60 (s, 3H), 3.21 (t, J = 4.8 Hz, 3H), 2.83 (s, 2H), 2.72-2.64 (m, |
| | 2H), 2.56 (t, J = 6.1 Hz, 2H), 2.42-2.28 (m, 3H), 2.11-1.94 (m, 1H), |
| | 1.86-1.78 (m, 2H), 1.74-1.68 (m, 2H), 1.60 (s, 2H), 1.46-1.39 (m, 1H). |
| 397 | (400 MHz, CDCl₃) δ 10.12 (s, 1H), 7.22 (d, J = 7.2 Hz, 1H), 7.01 (d, J = |
| | 6.0 Hz, 1H), 6.37 (d, J = 7.2 Hz, 1H), 4.39-4.34 (m, 1H), 3.58 (d, J = 4.0 |
| | Hz, 2H), 3.49 (d, J = 3.6 Hz, 2H), 2.96-2.94 (m, 1H), 2.73 (t, J = 6.0 Hz, |
| | 3H), 2.45 (d, J = 10.8 Hz, 1H), 2.33-2.25 (m, 2H), 2.14-2.03 (m, 3H), |
| | 1.91 (t, J = 5.2 Hz, 2H), 1.76-1.72 (m, 8H), 1.61-1.55 (m, 8H), 1.50- |
| | 1.43 (m, 2H). |
| 398 | (400 MHz, CDCl₃) a 10.17 (s, 1H), 7.25 (s, 1H), 6.83 (d, J = 5.2 Hz, 1H), |
| | 6.36 (d, J = 7.2 Hz, 1H), 4.35-4.29 (m, 1H), 3.82-3.75 (m, 1H), 3.50 (t, |
| | J = 5.6 Hz, 2H), 3.43-3.35 (m, 2H), 3.19 (d, J = 11.2 Hz, 1H), 2.99- |
| | 2.96 (m, 1H), 2.87-2.83 (m, 2H), 2.73 (t, J = 6.0 Hz, 3H), 2.40 (s, 1H), |
| | 2.29-2.23 (m, 2H), 1.95-1.91 (m, 2H), 1.79-1.70 (m, 8H), 1.65-1.55 |
| | (m, 10H), 0.89-0.82 (m, 1H). |
| 399 | (400 MHz, CDCl₃) δ 10.57 (s, 1H), 7.21 (d, J = 7.2 Hz, 1H), 6.75 (d, J = |
| | 5.6 Hz, 1H), 6.25 (d, J = 7.2 Hz, 1H), 4.38-4.33 (m, 1H), 3.78-3.72 (m, |
| | 1H), 3.50-3.45 (m, 3H), 3.43-3.38 (m, 1H), 3.07-2.97 (m, 1H), 2.73- |
| | 2.68 (m, 3H), 2.66-2.52 (m, 3H), 2.43-2.35 (m, 1H), 2.28-2.19 (m, |
| | 2H), 2.16-2.07 (m, 2H), 1.95-1.88 (m, 2H), 1.81-1.68 (m, 12H), 1.62- |
| | 1.57 (m, 8H), 1.51-1.45 (m, 1H). |
| 405 | (400 MHz, CDCl₃) δ 10.42 (br s, 1H), 8.62 (s, 1H), 7.89 (d, J = 8.0 Hz, |
| | 1H), 7.79 (br d, J = 8.4 Hz, 1H), 7.69 (t, J = 7.2 Hz, 1H), 7.43 (br t, J = 7.6 |
| | Hz, 1H), 7.33-7.28 (m, 1H), 7.23 (d, J = 7.2 Hz, 1H), 6.35-6.23 (m, |
| | 1H), 4.82-4.77 (m, 1H), 3.89-3.83 (m, 1H), 3.70-3.59 (m, 2H), 3.52- |
| | 3.48 (br s, 2H), 3.45-3.41 (m, 1H), 3.06-2.96 (m, 1H), 2.78-2.69 (m, |
| | 3H), 2.62-2.49 (m, 3H), 2.45-2.33 (m, 2H), 2.31-2.10 (m, 2H), 1.95- |
| | 1.89 (m, 2H), 1.85-1.71 (m, 4H), 1.67-1.41 (m, 4H). |
| 406 | (400 MHz, CDCl₃) δ 9.36 (br s, 1H), 8.60 (s, 1H), 7.91 (d, J = 8.0 Hz, |
| | 1H), 7.75-7.55 (m, 3H), 7.32-7.26 (m, 1H), 7.24 (d, J = 7.6 Hz, 1H), |
| | 6.39 (d, J = 7.6 Hz, 1H), 4.78 (q, J = 5.6 Hz, 1H), 3.85-3.77 (m, 1H), |
| | 3.62-3.53 (m, 1H), 3.51-3.41 (m, 3H), 3.05-2.83 (m, 5H), 2.72 (t, J = |
| | 6.0 Hz, 2H), 2.65-2.46 (m, 4H), 2.46-2.34 (m, 1H), 1.97-1.82 (m, 3H), |
| | 1.70-1.45 (m, 3H). |
| 407 | (400 MHz, DMSO-d₆) δ = 8.43 (s, 1H), 8.31 (d, J = 8.4 Hz, 1H), 8.19 (d, |
| | J = 7.6 Hz, 1H), 7.85-7.75 (m, 1H), 7.71-7.69 (m, 1H), 7.57-7.48 (m, |
| | 1H), 6.95 (d, J = 7.2 Hz, 1H), 6.35-6.26 (m, 2H), 4.83-4.78 (m, 1H), |
| | 3.63-3.58 (m, 2H), 3.22-3.17 (m, 5H), 2.80-2.76 (m, 1H), 2.56 (s, 3H), |
| | 2.13-2.09 (m, 1H), 1.88-1.81 (m, 2H), 1.75-1.69 (m, 2H), 1.63-1.55 |
| | (m, 1H), 1.33-1.28 (m, 1H), 1.23-1.22 (m, 2H), 1.16-1.06 (m, 1H). |
| 443 | (400 MHz, DMSO-d₆) δ 7.16 (d, J = 7.2 Hz, 1H), 7.09 (d, J = 7.2 Hz, |
| | 1H), 6.87 (s, 1H), 6.29 (d, J = 7.2 Hz, 1H), 4.20-4.14 (m, 1H), 3.33- |
| | 3.30 (m, 3H), 3.27-3.23 (m, 3H), 3.00 (d, J = 10.8 Hz, 1H), 2.61 (m, |
| | 3H), 2.47-2.42 (m, 3H), 2.19-2.13 (m, 1H), 2.08 (d, J = 11.2 Hz, 1H), |
| | 1.96-1.88 (m, 1H), 1.84-1.69 (m, 7H), 1.64-1.58 (m, 6H), 1.56-1.54 |
| | (m, 1H), 1.53-1.48 (m, 6H), 1.44-1.37 (m, 1H), 1.21-1.13 (m, 1H). |
| 444 | (400 MHz, DMSO-d₆) δ 8.44 (s, 1H), 8.32 (d, J = 8.4 Hz, 1H), 8.13 (d, J = |
| | 7.2 Hz, 1H), 7.79-7.75 (m, 1H), 7.68 (d, J = 7.6 Hz, 1H), 7.52-7.49 |
| | (m, 1H), 7.28 (s, 1H), 7.10 (d, J = 7.2 Hz, 1H), 6.29 (d, J = 7.2 Hz, 1H), |
| | 4.83-4.78 (m, 1H), 3.64-3.60 (m, 1H), 3.57-3.51 (m, 1H), 3.31-3.29 |
| | (m, 1H), 3.23 (t, J = 5.2 Hz, 2H), 3.17 (s, 1H), 3.10 (d, J = 8.0 Hz, 1H), |
| | 2.63-2.59 (t, J = 6.0 Hz, 3H), 2.43-2.41 (m, 2H), 2.34-2.31 (m, 1H), |
| | 2.20-2.17 (m, 1H), 2.09-2.04 (m, 2H), 1.80-1.68 (m, 6H), 1.67-1.62 |
| | (m, 2H), 1.40-1.31 (m, 1H), 1.17-1.10 (m, 1H). |
| 446 | (400 MHz, DMSO-d₆) δ 8.45 (s, 1H), 8.31 (d, J = 8.4 Hz, 1H), 8.16 (d, J = |
| | 7.6 Hz, 1H), 7.81-7.75 (m, 1H), 7.71-7.67 (m, 1H), 7.52-7.49 (m, |
| | 8.2 Hz, 1H), 7.03 (d, J = 7.2 Hz, 1H), 6.71 (s, 1H), 6.21 (d, J = 7.4 Hz, |
| | 1H), 4.83-4.73 (m, 1H), 3.64-3.61 (m, 1H), 3.58-3.53 (m, 1H), 3.31- |
| | 3.29 (m, 1H), 3.23 (t, J = 5.2 Hz, 2H), 2.87-2.79 (m, 1H), 2.62-2.56 (m, |
| | 3H), 2.34 (t, J = 7.6 Hz, 2H), 2.28-2.24 (m, 1H), 2.20-2.09 (m, 3H), |
| | 1.88-1.70 (m, 5H), 1.68-1.60 (m, 3H), 1.40-1.33 (m, 1H), 1.18-1.12 |
| | (m, 1H). |
| 447 | (400 MHz, DMSO-d₆) δ 7.21 (d, J = 7.6 Hz, 1H), 7.07 (d, J = 7.2 Hz, |
| | 1H), 6.75 (s, 1H), 6.28 (d, J = 7.2 Hz, 1H), 4.22-4.13 (m, 1H), 3.32- |
| | 3.29 (m, 3H), 3.24 (s, 2H), 2.90 (d, J = 9.6 Hz, 1H), 2.61 (t, J = 6.4 Hz, |
| | 3H), 2.45 (t, J = 7.6 Hz, 3H), 2.37-2.31 (m, 1H), 2.15-2.08 (m, 1H), |
| | 2.06-1.99 (m, 1H), 1.91-1.84 (m, 2H), 1.80-1.70 (m, 6H), 1.63-1.58 |
| | (m, 6H), 1.55 (d, J = 3.2 Hz, 1H), 1.53-1.48 (m, 6H), 1.42 (d, J = 3.6 |
| | 1H), 1.20 (s, 1H). |
| 466 | (400 MHz, DMSO-d₆) δ 8.40 (d, J = 7.6 Hz, 1H), 7.17-7.06 (m, 2H), |
| | 6.79 (d, J = 11.6 Hz, 2H), 6.30 (d, J = 7.2 Hz, 1H), 4.96-4.86 (m, 1H), |
| | 4.42-4.34 (m, 1H), 4.17 (d, J = 12.8 Hz, 1H), 3.96 (dd, J₁ = 11.2 Hz, J₂ = |
| | 3.6 Hz, 1H), 3.78-3.72 (m, 1H), 3.59-3.41 (m, 5H), 3.35-3.20 (m, 5H), |
| | 3.06 (d, J = 8.4 Hz, 1H), 2.61 (t, J = 6.4 Hz, 3H), 2.46-2.38 (m, 3H), 2.35- |
| | 2.31 (m, 1H), 2.04-1.94 (m, 2H), 1.85-1.73 (m, 5H), 1.70-1.66 (m, |
| | 1H), 1.45-1.34 (m, 1H), 1.24-1.14 (m, 1H). |
| 467 | (400 MHz, DMSO-d₆) δ 8.29 (d, J = 8.0 Hz, 1H), 7.10 (d, J = 8.0 Hz, |
| | 2H), 6.65 (d, J = 12.0 Hz, 2H), 6.29 (d, J = 4.0 Hz, 1H), 4.35 (q, J = 8.0 |
| | Hz, 1H), 3.70 (t, J = 4.0 Hz, 4H), 3.53 (t, J = 8.0 Hz, 2H), 3.25 (d, J = 4.0 |
| | Hz, 4H), 3.20 (t, J = 4.0 Hz, 4H), 3.04 (s, 1H), 2.61 (t, J = 8.0 Hz, 4H), |
| | 2.45-2.41 (m, 4H), 2.10-1.91 (m, 4H), 1.82-1.66 (m, 6H). |
| 468 | (400 MHz, DMSO-d₆) δ 8.69 (d, J = 7.6 Hz, 1H), 7.54-7.45 (m, 1H), |
| | 7.27 (s, 1H), 7.17-7.09 (m, 3H), 6.31 (d, J = 7.2 Hz, 1H), 4.47-4.41 (m, |
| | 1H), 3.58-3.47 (m, 5H), 3.34-3.30 (m, 1H), 3.26 (t, J = 5.2 Hz, 2H), |
| | 3.10 (d, J = 9.2 Hz, 1H), 2.62 (t, J = 6.0 Hz, 3H), 2.46-2.39 (m, 2H), |
| | 2.38-2.33 (m, 1H), 2.11-1.94 (m, 3H), 1.84-1.72 (m, 5H), 1.46-1.38 |
| | (m, 1H), 1.24-1.17 (m, 1H). |
| 469 | (400 MHz, DMSO-d₆) δ 8.37 (d, J = 7.6 Hz, 1H), 7.85 (d, J = 7.2 Hz, |
| | 2H), 7.53 (t, J = 7.6 Hz, 1H), 7.46 (t, J = 7.6 Hz, 2H), 7.23 (s, 1H), 7.11 |
| | (d, J = 7.2 Hz, 1H), 6.30 (d, J = 7.6 Hz, 1H), 4.50-4.42 (m, 1H), 3.59- |
| | 3.56 (m, 2H), 3.53-3.50 (m, 2H), 3.35-3.29 (m, 2H), 3.24 (t, J = 5.2 Hz, |
| | 2H), 3.09 (d, J = 10.0 Hz, 1H), 2.61 (t, J = 6.0 Hz, 3H), 2.47-2.39 (m, |
| | 2H), 2.37-2.30 (m, 1H), 2.10-2.01 (m, 2H), 1.97-1.89 (m, 2H), 1.82- |
| | 1.71 (m, 4H), 1.46-1.34 (m, 1H), 1.24-1.12 (m, 1H). |
| 470 | (400 MHz, DMSO-d₆) δ 8.42 (s, 1H), 8.11 (d, J = 5.2 Hz, 1H), 7.74 (d, J = |
| | 7.2 Hz, 1H), 7.38 (d, J = 5.2 Hz, 1H), 7.04 (d, J = 7.2 Hz, 1H), 6.73 (s, |
| | 1H), 6.23 (d, J = 7.2 Hz, 1H), 4.76-4.65 (m, 1H), 3.62-3.52 (m, 2H), |
| | 3.30-3.20 (m, 4H), 2.85 (d, J = 10.4 Hz, 1H), 2.60 (t, J = 6.0 Hz, 3H), |
| | 2.36 (t, J = 7.6 Hz, 2H), 2.31-2.25 (m, 1H), 2.20-2.16 (m, 1H), 2.12- |
| | 2.02 (m, 2H), 1.99-1.93 (m, 1H), 1.86-1.78 (m, 2H), 1.77-1.71 (m, |
| | 2H), 1.68-1.62 (m, 2H), 1.44-1.31 (m, 1H), 1.20-1.08 (m, 1H). |
| 472 | (400 MHz, DMSO-d₆) δ 8.31 (s, 1H), 7.93-7.88 (m, 1H), 7.72-7.69 (m, |
| | 1H), 7.59-7.54 (m, 1H), 7.42-7.32 (m, 1H), 7.12 (d, J = 7.2 Hz, 1H), |
| | 6.30 (d, J = 7.2 Hz, 1H), 4.80-4.74 (m, 1H), 3.63 (d, J = 4.8 Hz, 1H), |
| | 3.55 (d, J = 4.4 Hz, 1H), 3.25 (s, 2H), 3.16-3.14 (m, 1H), 2.70-2.61 (m, |
| | 3H), 2.60 (s, 2H), 2.42 (s, 2H), 2.34-2.32 (m, 2H), 2.17 (s, 1H), 1.99- |
| | 1.93 (m, 2H), 1.88-1.82 (m, 2H), 1.78-1.74 (m, 2H), 1.65 (s, 2H), 1.38- |
| | 1.33 (m, 1H), 1.15 (s, 1H). |
| 473 | (400 MHz, DMSO-d₆) δ 8.28 (s, 1H), 7.31-7.20 (m, 1H), 7.18 (s, 1H), |
| | 7.12 (d, J = 8.0 Hz, 1H), 7.07-7.02 (m, 1H), 6.32 (d, J = 4.0 Hz, 1H), |
| | 4.55-4.43 (m, 1H), 3.58 (t, J = 4.0 Hz, 2H), 2.28-2.23 (m, 2H), 2.92 (d, |
| | J = 12.0 Hz, 1H), 2.64-2.58 (m, 6H), 2.53-2.51 (m, 4H), 2.25-2.05 (m, |
| | 3H), 2.03-1.95 (m, 1H), 1.90-1.63 (m, 7H), 1.50-1.37 (m, 2H). |
| 474 | (400 MHz, DMSO-d₆) δ 8.52-8.40 (m, 2H), 8.39-8.24 (m, 1H), 7.69- |
| | 7.56 (m, 1H), 7.12 (d, J = 7.2 Hz, 1H), 6.30 (d, J = 7.2 Hz, 1H), 4.83- |
| | 4.71 (m, 1H), 3.60 (s, 1H), 3.23-3.21 (m, 2H), 3.13-3.10 (m, 1H), 2.60 |
| | (d, J = 6.0 Hz, 2H), 2.42 (d, J = 8.4 Hz, 4H), 2.33 (s, 2H), 2.27-2.17 (m, |
| | 2H), 2.10-2.01 (m, 2H), 1.90 (s, 2H), 1.78-1.72 (m, 4H), 1.64 (d, J = |
| | 2.8 Hz, 2H), 1.39-1.33 (m, 1H), 1.19-1.11 (m, 1H). |
| 475 | (400 MHz, DMSO-d₆) δ 8.60-8.53 (m, 2H), 8.01-7.88 (m, 1H), 7.80- |
| | 7.65 (m, 1H), 7.02 (d, J = 7.2 Hz, 1H), 6.68 (s, 1H), 6.21 (d, J = 7.2 Hz, |
| | 1H), 4.85-4.73 (m, 1H), 3.63-3.61 (m, 2H), 3.23 (t, J = 5.2 Hz, 3H), |
| | 2.86 (s, 1H), 2.59 (t, J = 6.0 Hz, 3H), 2.38-2.31 (m, 3H), 2.28-2.21 (m, |
| | 1H), 2.18-2.04 (m, 3H), 1.98-1.87 (m, 1H), 1.81-1.68 (m, 4H), 1.67- |
| | 1.58 (m, 2H), 1.44-1.32 (m, 1H), 1.21-1.13 (m, 1H). |
| 479 | (400 MHz, DMSO-d₆) δ 8.39 (d, J = 7.6 Hz, 1H), 7.85 (d, J = 7.2 Hz, |
| | 2H), 7.53 (t, J = 7.2 Hz, 1H), 7.45 (t, J = 7.6 Hz, 2H), 7.06 (d, J = 7.2 Hz, |
| | 1H), 6.84 (s, 1H), 6.25 (d, J = 7.2 Hz, 1H), 4.45-4.40 (m, 1H), 3.59- |
| | 3.49 (m, 4H), 3.35-3.30 (m, 1H), 3.24 (t, J = 5.2 Hz, 2H), 2.87 (d, J = |
| | 9.2 Hz, 1H), 2.60 (t, J = 6.0 Hz, 3H), 2.41 (t, J = 7.6 Hz, 2H), 2.36-2.26 |
| | (m, 2H), 2.07-1.93 (m, 4H), 1.77-1.69 (m, 4H), 1.43-1.34 (m, 1H), |
| | 1.24-1.14 (m, 1H). |
| 480 | (400 MHz, DMSO-d₆) δ 8.73 (d, J = 7.6 Hz, 1H), 7.54-7.44 (m, 1H), |
| | 7.13 (t, J = 7.6 Hz, 2H), 7.06 (d, J = 7.6 Hz, 1H), 6.82 (s, 1H), 6.27 (d, J = |
| | 7.6 Hz, 1H), 4.45-4.37 (m, 1H), 3.57-3.49 (m, 2H), 3.38-3.31 (m, 1H), |
| | 3.24 (t, J = 5.2 Hz, 2H), 2.91 (d, J = 10.4 Hz, 1H), 2.60 (t, J = 6.0 Hz, |
| | 3H), 2.43 (t, J = 7.6 Hz, 3H), 2.37-2.33 (m, 1H), 2.07-1.95 (m, 3H), |
| | 1.88-1.72 (m, 6H), 1.66 (m, 1H), 1.43-1.34 (m, 1H), 1.20-1.17 (m, 1H). |
| 494 | (400 MHz, DMSO-d₆) δ 8.49 (d, J = 7.6 Hz, 1H), 7.06 (d, J = 7.2 Hz, |
| | 1H), 6.83-6.68 (m, 3H), 6.27 (d, J = 7.6 Hz, 1H), 4.97-4.84 (m, 1H), |
| | 4.43-4.32 (m, 1H), 4.17 (d, J = 12.8 Hz, 1H), 3.96 (dd, J₁ = 11.2 Hz, J₂ = |
| | 3.2 Hz, 1H), 3.75 (d, J = 11.6 Hz, 1H), 3.58-3.36 (m, 6H), 3.28-3.22 |
| | (m, 3H), 2.93 (d, J = 9.6 Hz, 1H), 2.60 (t, J = 6.0 Hz, 2H), 2.44 (t, J = 7.6 |
| | Hz, 4H), 2.15 (s, 2H), 2.01-1.94 (m, 1H), 1.91-1.83 (m, 1H), 1.81- |
| | 1.67 (m, 6H), 1.47-1.37 (m, 1H), 1.26-1.21 (m, 1H). |
| 497 | (400 MHz, DMSO-d₆) δ 8.59-8.53 (m, 2H), 7.95 (s, 1H), 7.78-7.69 (m, |
| | 1H), 7.56-7.25 (m, 1H), 7.12 (d, J = 7.6 Hz, 1H), 6.30 (d, J = 7.6 Hz, |
| | 1H), 4.86-4.78 (m, 1H), 3.63-3.59 (m, 2H), 3.23 (t, J = 5.2 Hz, 3H), |
| | 3.15-3.12 (m, 1H), 2.61 (t, J = 6.0 Hz, 3H), 2.45-2.39 (m, 3H), 2.34- |
| | 2.31 (m, 1H), 2.28-2.03 (m, 4H), 1.85-1.72 (m, 4H), 1.70-1.60 (m, |
| | 2H), 1.41-1.33 (m, 1H), 1.19-1.11 (m, 1H). |
| 498 | (400 MHz, DMSO-d₆) δ 8.32 (s, 1H), 7.90 (d, J = 8.0 Hz, 1H), 7.71 (d, J = |
| | 6.0 Hz, 1H), 7.57 (d, J = 6.0 Hz, 1H), 7.44-7.31 (m, 1H), 7.12 (d, J = |
| | 7.2 Hz, 1H), 6.30 (d, J = 7.2 Hz, 1H), 4.82-4.69 (m, 1H), 3.61-3.59 (m, |
| | 1H), 3.53 (s, 1H), 3.24 (s, 2H), 3.13 (d, J = 8.0 Hz, 1H), 2.63-2.59 (m, |
| | 3H), 2.57-2.55 (m, 2H), 2.44-2.37 (m, 3H), 2.30-2.25 (m, 1H), 2.19- |
| | 2.13 (m, 1H), 2.00-1.92 (m, 2H), 1.85-1.79 (m, 2H), 1.77-1.73 (m, |
| | 2H), 1.68-1.59 (m, 2H), 1.41-1.31 (m, 1H), 1.14-1.07 (m, 1H). |
| 502 | (400 MHz, DMSO-d₆) δ 8.27 (s, 1H), 7.21-7.14 (m, 2H), 7.12 (d, J = 8.0 |
| | Hz, 1H), 7.04-6.87 (m, 1H), 6.32 (d, J = 8.0 Hz, 1H), 4.42 (d, J = 4.0 Hz, |
| | 1H), 3.65-3.62 (m, 1H), 3.25 (t, J = 4.0 Hz, 3H), 2.99 (d, J = 8.0 Hz, |
| | 1H), 2.68-2.66 (m, 1H), 2.62 (t, J = 8.0 Hz, 3H), 2.59 (s, 3H), 2.54-2.52 |
| | (m, 2H), 2.47-2.42 (m, 2H), 2.18-2.09 (m, 2H), 1.97-1.65 (m, 7H), |
| | 1.52-1.25 (m, 3H). |
| 503 | (400 MHz, DMSO-d₆) δ 8.50-8.43 (m, 1H), 8.41 (s, 1H), 8.38-8.20 (m, |
| | 1H), 7.62 (s, 1H), 7.12 (d, J = 7.2 Hz, 1H), 6.30 (d, J = 7.2 Hz, 1H), 4.77 |
| | (d, J = 2.8 Hz, 1H), 3.59 (s, 1H), 3.23 (s, 2H), 3.14-3.11 (m, 1H), 2.61 |
| | (s, 2H), 2.47-2.38 (m, 4H), 2.37-2.27 (m, 2H), 2.20 (d, J = 2.4 Hz, 2H), |
| | 2.09-2.00 (m, 2H), 1.90 (s, 2H), 1.77-1.71 (m, 4H), 1.69-1.58 (m, 2H), |
| | 1.37-1.32 (m, 1H), 1.14 (s, 1H), |
| 511 | (400 MHz, DMSO-d₆) δ 8.41 (s, 1H), 8.10 (d, J = 5.2 Hz, 1H), 7.70 (d, J = |
| | 7.2 Hz, 1H), 7.38 (d, J = 5.2 Hz, 1H), 7.19 (s, 1H), 7.10 (d, J = 7.6 Hz, |
| | 1H), 6.30 (d, J = 7.2 Hz, 1H), 4.77-4.64 (m, 1H), 3.61-3.58 (m, 2H), |
| | 3.30-3.28 (m, 1H), 3.26-3.21 (m, 3H), 3.10 (d, J = 9.2 Hz, 1H), 2.61 (t, |
| | J = 5.6 Hz, 3H), 2.46-2.27 (m, 4H), 2.19-2.11 (m, 1H), 2.05-1.96 (m, |
| | 2H), 1.89-1.79 (m, 2H), 1.77-1.62 (m, 4H), 1.41-1.31 (m, 1H), 1.15- |
| | 1.08 (m, 1H). |
| 512 | (400 MHz, DMSO-d₆) δ 8.28 (d, J = 8.0 Hz, 1H), 7.05 (d, J = 8.0 Hz, |
| | 1H), 6.78 (s, 1H), 6.64 (d, J = 12.0 Hz, 2H), 6.27 (d, J = 8.0 Hz, 1H), 4.32 |
| | (q, J = 8.0 Hz, 1H), 3.69 (t, J = 4.0 Hz, 4H), 3.52 (s, 2H), 3.35 (t, J = 4.0 |
| | Hz, 2H), 3.24 (t, J = 4.0 Hz, 2H), 3.21 (t, J = 4.0 Hz, 4H), 2.90 (d, J = 8.0 |
| | Hz, 1H), 2.60 (t, J = 4.0 Hz, 4H), 2.45-2.36 (m, 4H), 2.11-1.83 (m, 4H), |
| | 1.75-1.65 (m, 6H). |

The following compounds, set forth in Table 9, were prepared according to the general procedures provided in Schemes 1, 22, 27, and 28 or analogous procedures thereto.

TABLE 9

| Compound | |
|---|---|
| No. | ¹H NMR Data |
| 329 | (400 MHz, DMSO-d6) δ 7.65-7.61 (m, 2H), 7.55-7.52 (m, 2H), 7.47- |
| | 7.34 (m, 4H), 7.27 (d, J = 7.6 Hz, 1H), 7.04 (d, J = 7.2 Hz, 1H), 6.73 (s, |
| | 1H), 6.26 (d, J = 7.2 Hz, 1H), 3.69 (t, J = 7.2 Hz, 1H), 3.43-3.34 (m, |
| | 2H), 3.22-3.20 (m, 2H), 2.94-2.88 (m, 1H), 2.59 (t, J = 6.0 Hz, 4H), |
| | 2.44-2.40 (m. 2H), 2.28-2.22 (m, 2H), 1.90-1.78 (m, 4H), 1.77-1.63 |
| | (m, 6H), 1.41-1.30 (m, 1H), 1.11-1.02 (m, 1H). |
| 330 | (400 MHz, DMSO-d6) δ 7.62-7.58 (m, 2H), 7.52 (s, 1H), 7.44 (t, J = 7.2 |
| | Hz, 3H), 7.36-7.28 (m, 2H), 7.24 (d, J = 7.6 Hz, 1H), 7.00 (d, J = 7.2 Hz, |
| | 1H), 6.42 (s, 1H), 6.22 (d, J = 7.2 Hz, 1H), 3.38-3.27 (m, 4H), 3.24- |
| | 3.14 (m, 4H), 2.84 (d, J = 8.4 Hz, 1H), 2.58 (d, J = 6.0 Hz, 2H), 2.38 (t, J = |
| | 7.6 Hz, 2H), 2.23-2.18 (m, 2H), 1.84 (s, 1H), 1.81-1.58 (m, 8H), 1.39- |
| | 1.28 (m, 1H), 1.10-0.98 (m, 1H). |
| 652 | (400 MHz, DMSO-d6) δ 7.10 (t, J = 7.6 Hz, 1H), 7.04 (d, J = 7.2 Hz, 1H), |
| | 6.85 (s, 1H), 6.79 (d, J = 8.4 Hz, 1H), 6.71-6.66 (m, 2H), 6.26 (d, J = 7.2 |
| | Hz. 1H), 3.59-3.48 (m, 3H), 3.43-3.36 (m, 1H), 3.29 (s. 1H), 3.25- |
| | 3.21 (m, 4H), 3.17-3.12 (m, 1H), 3.01 (s, 2H), 2.98-2.93 (m, 1H), 2.70- |
| | 2.63 (m, 1H), 2.62-2.58 (m, 3H), 2.47-2.39 (m, 3H), 2.31 (d, J = 6.8 |
| | Hz, 2H), 2.14-2.05 (m, 2H), 1.91 (s, 1H), 1.83-1.77 (m, 2H), 1.76- |
| | 1.72 (m, 2H), 1.65-1.57 (m, 1H), 1.41-1.32 (m, 1H), 1.23 (s, 1H), 1.07 |
| | (s, 6H). |

### Biological Evaluation

The compounds exemplified in this document were tested for their ability to inhibit αvβ1 and αvβ6 in below described solid phase integrin assays. The assays result of the examples are listed in Table 1.

### Solid Phase Integrin αvβ1 Assay

96-well microtiter plates (4 HBX Immulon; Thermo Fisher Scientific, Waltham, MA) were coated with 100 μL/well of 1 μg/mL recombinant TGFb1-LAP in TBS at 4° C., overnight. The coating solution was removed, and plates were blocked with 200 μL/well of blocking and binding buffer (2% BSA/TBST, 1 mM MnCl₂) at room temperature for 1 hr. Blocking buffer was removed and 50 μL of binding buffer and testing compounds were added. 50 μL of diluted αvβ1 (0.2 ug/mL in binding buffer) was added to wells (100 μL/well total) for and plates incubated for 90 min at room temperature. Wells were washed thrice with washing buffer (TBS, 0.05% Tween, 1 mM MnCl₂) and plates were incubated with 100 μL/well of a 1:12500 dilution of streptavidin-horseradish peroxidase conjugate (Thermo Fisher Scientific) in binding buffer for 20 min at room temperature. Bound protein was detected using the substrate TMB. The IC₅₀ values for testing compounds were calculated by Levenberg-Marquardt four parameter fitting logistics.

### Solid Phase Integrin αvβ6 Assay

96-well microtiter plates (4 HBX Immulon; Thermo Fisher Scientific, Waltham, MA) were coated with 100 μL/well of 1 μg/mL recombinant TGFb1-LAP in TBS at 4° C., overnight. The coating solution was removed, and plates were blocked with 200 μL/well of blocking and binding buffer (2% BSA/TBST, 1 mM MnCl₂) at room temperature for 1 hr. Blocking buffer was removed and 50 uL of binding buffer and testing compounds were added. 50 μL of diluted αvβ6 (0.2 μg/mL in binding buffer) was added to wells (100 uL/well total) and plates incubated for 1 hr at room temperature. Wells were washed thrice with washing buffer (TBS, 0.05% Tween, 1 mM MnCl₂) and plates were incubated with 100 μL/well of a 1:12500) dilution of streptavidin-horseradish peroxidase conjugate (Thermo Fisher Scientific) in binding buffer for 20 min at room temperature. Bound protein was detected using the substrate TMB. The IC₅₀ values for testing compounds were calculated by Levenberg-Marquardt four parameter fitting logistics.

Table 10, below, reports the biological activity of compounds 201 to 299 as measured by the Solid Phase Integrin αvβ1 Assay and the Solid Phase Integrin αvβ6 Assay above.

TABLE 10

| Compound No. | αVβ1 IC₅₀ | αVβ6 IC₅₀ |
|---|---|---|
| 201-A | B | A |
| 201-B | A | A |
| 202 | A | A |
| 203-A | C | B |
| 203-B | A | A |
| 204-A | A | A |
| 204-B | A | A |
| 205 | A | A |
| 206 | A | A |
| 207 | B | B |
| 208-A | A | A |
| 208-B | C | B |
| 209-A | A | A |
| 209-B | C | B |
| 210 | B | B |
| 211-A | A | A |
| 211-B | C | B |
| 212-A | C | C |
| 212-B | B | A |
| 213 | A | A |
| 214 | A | A |
| 215-A | A | A |
| 215-B | A | A |
| 216-A | B | A |
| 216-B | C | C |
| 217 | A | A |
| 218 | A | A |
| 219 | A | A |
| 220 | A | A |
| 221 | A | A |
| 222 | A | A |
| 223 | A | A |
| 224 | A | A |
| 225 | A | A |
| 226 | A | A |
| 227 | A | A |
| 228 | A | A |
| 229 | A | A |
| 230 | A | A |
| 231 | A | A |
| 232 | A | A |
| 233 | A | A |
| 234 | A | A |
| 235 | A | A |
| 236 | A | A |
| 237 | A | A |
| 238 | A | A |
| 239 | A | A |
| 240 | A | A |
| 241-A | A | A |
| 241-B | A | A |
| 242 | A | A |
| 243 | A | A |
| 244-A | A | A |
| 244-B | A | A |
| 245-A | B | A |
| 245-B | A | A |
| 246 | A | A |
| 247 | A | A |
| 248-A | B | B |
| 248-B | A | A |
| 249-A | A | A |
| 249-B | A | A |
| 250-A | A | A |
| 250-B | B | C |
| 251-A | B | B |
| 251-B | A | A |
| 252-A | A | B |
| 252-B | C | A |
| 253-A | A | A |
| 253-B | B | C |
| 254-A | A | B |
| 254-B | A | A |
| 255 | A | A |
| 256-A | B | B |
| 256-B | A | A |
| 257-A | A | A |
| 257-B | A | A |
| 258-A | B | B |
| 258-B | A | A |
| 259-A | A | A |
| 259-B | A | B |
| 260-A | A | A |
| 260-B | B | C |
| 261 | A | A |
| 262 | A | A |
| 263 | A | A |
| 264 | A | A |
| 265 | A | A |
| 266 | A | A |
| 267 | A | A |
| 268 | A | A |
| 269 | C | B |
| 270 | A | A |
| 271 | A | A |
| 272 | A | A |
| 273 | A | A |
| 274 | A | A |
| 275 | B | A |
| 276 | A | A |
| 277 | A | A |
| 278 | A | A |
| 279 | A | A |
| 280 | A | A |
| 281 | A | A |
| 282 | A | A |
| 283 | A | A |
| 284 | A | A |
| 285 | A | A |
| 286 | C | C |
| 287 | C | C |
| 288 | B | A |
| 289 | B | B |
| 290 | C | B |
| 291-A | B | A |
| 291-B | B | B |
| 292 | C | C |
| 293 | C | C |
| 294 | C | C |
| 295 | A | A |
| 296 | B | C |
| 297 | A | A |
| 297-A | B | B |
| 297-B | A | A |
| 298-A | A | B |
| 298-B | A | A |
| 299 | C | C |
| 300 | A | |
| 301-A | C | C |
| 301-B | C | C |
| 302 | A | A |
| 303 | A | A |
| 304 | A | A |
| 305 | A | A |
| 306 | B | B] |
| 307 | A | A |
| 308 | B | B |
| 309 | A | A |
| 310 | | B |
| 311 | A | A |
| 312 | A | A |
| 313 | A | A |
| 314 | B | B |
| 315 | A | A |
| 316 | B | C |
| 317 | A | A |
| 318 | B | C |
| 319 | A | A |
| 320 | C | C |
| 321 | A | B |
| 322 | B | C |
| 323 | A | A |
| 324 | A | C |
| 325 | A | A |
| 326 | A | A |
| 327 | A | A |
| 328 | B | B |
| 329 | C | C |
| 330 | C | A |
| 331 | A | A |
| 332 | C | B |
| 333 | B | B |
| 334 | A | A |
| 335 | B | B |
| 336 | C | B |
| 337 | B | B |
| 338 | A | A |
| 339 | A | A |
| 340 | B | B |
| 341 | C | C |
| 342 | A | A |
| 343 | | A |
| 344 | A | A |
| 345 | C | C |
| 346 | A | A |
| 347 | A | A |
| 348 | A | A |
| 349 | C | C |
| 350 | C | C |
| 351 | C | C |
| 352 | C | C |
| 353 | C | B |
| 354 | C | C |
| 355 | B | C |
| 356 | B | C |
| 357 | B | B |
| 358 | A | C |
| 359 | A | A |
| 360 | A | A |
| 361 | A | A |
| 362 | A | A |
| 363 | A | A |
| 364 | A | A |
| 365 | A | A |
| 366 | A | A |
| 367 | A | A |
| 368 | A | A |
| 369 | A | A |
| 370 | | A |
| 371 | A | A |
| 372 | A | A |
| 373 | A | A |
| 374 | B | A |
| 375 | A | A |
| 376 | A | A |
| 377 | A | A |
| 378 | C | B |
| 379 | A | A |
| 380 | B | A |
| 381 | A | A |
| 382 | C | C |
| 383 | A | B |
| 384 | C | C |
| 385 | C | B |
| 386 | C | C |
| 387 | A | B |
| 388 | B | A |
| 389 | C | C |
| 390 | C | C |
| 391 | C | C |
| 392 | C | C |
| 393 | C | C |
| 394 | C | C |
| 395 | C | C |
| 396 | C | C |
| 397 | B | C |
| 398 | A | A |
| 399 | A | A |
| 400 | A | A |
| 401 | C | C |
| 402 | B | A |
| 403 | C | C |
| 404 | C | C |
| 405 | B | A |
| 406 | B | B |
| 407 | C | C |
| 408 | C | C |
| 409 | C | A |
| 410 | C | C |
| 411 | C | C |
| 412 | A | A |
| 413 | A | A |
| 414 | A | C |
| 415 | B | B |
| 416 | C | C |
| 417 | A | B |
| 418 | C | C |
| 419 | C | C |
| 420 | C | C |
| 421 | C | C |
| 422 | C | C |
| 423 | C | C |
| 424 | C | C |
| 425 | C | C |
| 426 | A | A |
| 427 | C | C |
| 428 | A | A |
| 429 | C | C |
| 430 | A | A |
| 431 | C | C |
| 432 | A | A |
| 433 | C | C |
| 434 | A | A |
| 435 | A | B |
| 436 | B | C |
| 437 | A | C |
| 438 | A | A |
| 439 | A | B |
| 440 | B | C |
| 441 | A | C |
| 442 | B | B |
| 443 | B | B |
| 444 | C | C |
| 445 | B | B |
| 446 | B | A |
| 447 | A | A |
| 448 | A | A |
| 449 | A | A |
| 450 | C | B |
| 451 | A | A |
| 452 | C | C |
| 453 | C | C |
| 454 | C | C |
| 455 | C | C |
| 456 | A | A |
| 457 | C | B |
| 458 | A | A |
| 459 | C | B |
| 460 | C | C |
| 461 | C | C |
| 462 | A | A |
| 463 | B | B |
| 464 | B | B |
| 465 | A | A |
| 466 | A | B |
| 467 | A | A |
| 468 | A | A |
| 469 | A | A |
| 470 | B | B |
| 471 | B | B |
| 472 | C | A |
| 473 | C | C |
| 474 | B | A |
| 475 | C | C |
| 476 | A | A |
| 477 | C | C |
| 478 | C | C |
| 479 | B | B |
| 480 | B | B |
| 481 | A | A |
| 482 | A | A |
| 483 | A | A |
| 484 | C | C |
| 485 | C | C |
| 486 | B | A |
| 487 | A | A |
| 488 | A | A |
| 489 | A | A |
| 490 | A | A |
| 491 | A | A |
| 492 | A | A |
| 493 | B | B |
| 494 | C | C |
| 495 | A | C |
| 496 | C | B |
| 497 | B | A |
| 498 | A | A |
| 499 | C | A |
| 500 | C | B |
| 501 | C | B |
| 502 | A | A |
| 503 | C | B |
| 504 | A | A |
| 505 | C | C |
| 506 | A | A |
| 507 | A | A |
| 508 | A | A |
| 509 | A | A |
| 510 | C | C |
| 511 | A | A |
| 512 | C | C |
| 513 | A | A |
| 514 | A | A |
| 515 | B | A |
| 516 | A | A |
| 517 | A | A |
| 518 | A | A |
| 519 | A | A |
| 520 | A | B |
| 521 | A | B |
| 522 | A | B |
| 523 | B | A |
| 524 | A | B |
| 525 | A | B |
| 526 | A | |
| 527 | B | B |
| 528 | C | C |
| 529 | B | B |
| 530 | C | C |
| 531 | C | C |
| 532 | C | C |
| 533 | A | A |
| 534 | A | A |
| 535 | A | A |
| 536 | A | A |
| 537 | A | |
| 538 | A | A |
| 539 | A | A |
| 540 | B | B |
| 541 | A | A |
| 542 | B | A |
| 543 | A | A |
| 544 | A | A |
| 545 | | A |
| 546 | A | A |
| 547 | A | A |
| 548 | | A |
| 549 | A | A |
| 550 | | A |
| 551 | B | A |
| 552 | B | B |
| 553 | B | B |
| 554 | C | B |
| 555 | B | B |
| 556 | B | B |
| 557 | C | B |
| 558 | A | |
| 559 | C | B |
| 560 | B | B |
| 561 | A | A |
| 562 | A | A |
| 563 | A | A |
| 564 | | A |
| 565 | A | A |
| 566 | A | A |
| 567 | A | A |
| 568 | A | A |
| 569 | B | B |
| 570 | A | A |
| 571 | A | A |
| 572 | A | A |
| 573 | B | B |
| 574 | A | A |
| 575 | B | A |
| 576 | A | A |
| 577 | A | A |
| 578 | A | A |
| 579 | A | A |
| 580 | A | A |
| 581 | B | B |
| 582 | A | A |
| 583 | A | A |
| 584 | A | A |
| 585 | A | A |
| 586 | A | A |
| 587 | A | A |
| 588 | A | A |
| 589 | A | A |
| 590 | | A |
| 591 | | A |
| 592 | | A |
| 593 | A | A |
| 594 | | B |
| 595 | A | A |
| 596 | | A |
| 597 | A | A |
| 598 | | C |
| 599 | A | A |
| 600 | | B |
| 601 | A | A |
| 602 | | B |
| 603 | A | A |
| 604 | A | A |
| 605 | A | A |
| 606 | A | B |
| 607 | A | A |
| 608 | C | C |
| 609 | C | C |
| 610 | C | C |
| 611 | C | C |
| 612 | C | C |
| 613 | A | A |
| 614 | A | A |
| 615 | A | A |
| 616 | A | A |
| 617 | A | A |
| 618 | A | A |
| 619 | A | A |
| 620 | A | A |
| 621 | A | A |
| 622 | B | B |
| 623 | A | A |
| 624 | B | C |
| 625 | C | B |
| 626 | A | A |
| 627 | A | A |
| 628 | A | A |
| 629 | C | B |
| 630 | C | C |
| 631 | A | A |
| 632 | B | B |
| 633 | A | A |
| 634 | B | B |
| 635 | B | B |
| 636 | A | A |
| 637 | B | A |
| 638 | A | A |
| 639 | B | B |
| 640 | B | B |
| 641 | B | B |
| 642 | C | C |
| 643 | A | A |
| 644 | B | B |
| 645 | B | C |
| 646 | C | B |
| 647 | A | A |
| 648 | B | B |
| 649 | A | A |
| 650 | A | A |
| 651 | A | A |
| 652 | A | A |

A: IC₅₀ <100 nM;
B: IC₅₀ 100-1000 nM;
C: IC₅₀ >1000 nM

What is claimed is:

1. A compound of Formula (I): 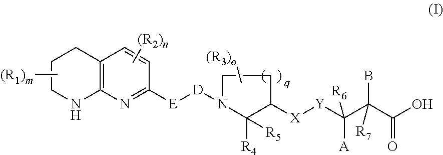
or pharmaceutically acceptable salts, hydrates or solvates thereof wherein:
each R₁ is independently hydrogen, alkyl, substituted alkyl, alkenyl, substituted alkenyl, alkynyl, substituted alkynyl, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl or substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl, substituted heteroarylalkynyl,
halo, —C(O)NR₈R₉, —C(O)OR₁₀, —NR₁₁C(O)OR¹², —NR₁₃C(O)OR¹⁴, —OC(O)OR₁₅, —CN, —CF₃, —NR₁₆S O₂R₁₇ or —OR₁₈;
m is 0, 1, 2 or 3;
each R₂ is independently hydrogen, alkyl, substituted alkyl, alkenyl, substituted alkenyl, alkynyl, substituted alkynyl, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl, substituted heteroarylalkynyl,
halo, —C(O)NR₁₉R₂₀), —C(O)OR₂₁, —NR₂₂C(O)OR²³, —NR₂₄C(O)OR₂₅, —OC(O)OR²⁶, —CN, —CF₃, —NR₂₇SO₂R₂₈ or —OR²⁹;
n is 0, 1 or 2;
each R₃ is independently hydrogen, alkyl, substituted alkyl, alkenyl, substituted alkenyl, alkynyl, substituted alkynyl, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl, substituted heteroarylalkynyl,
halo, —C(O)NR₃₀R₃₁, —C(O)OR₃₂, —NR₃₃C(O)OR₃₄, —NR₃₅C(O)OR₃₆, —OC(O)OR₃₇, —CN, —CF₃, —NR₃₈SO₂R₃₉ or —OR⁴⁰;
q is 0, 1, 2 or 3;
o is 0, 1 or 2 when q is 0;
o is 0, 1, 2 or 3 when q is 1;
o is 0, 1, 2, 3 or 4 when q is 2;
o is 0, 1, 2, 3, 4 or 5 when q is 3;
R₄ is hydrogen, alkyl, substituted alkyl, alkenyl, substituted alkenyl, alkynyl, substituted alkynyl, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl, substituted heteroarylalkynyl, —F, —C(O)NR₄₁R₄₂, —C(O)R₄₃, —C(O)OR₄₄, —CN, —CF₃ or R₄ and R₅ together with the atom to which they are bonded form a C₄-C₈ cycloalkyl ring;
E is —CH₂— or —CH₂Z—;
Z is —NR₄₆—, —S—, —SO₂— or —O—;
D is —(CH₂)₂—, —(CH₂)₃—, —CH═CHCH₂—, —C(O)—, —C≡CCH₂—, phenyl, cyclohexyl or cyclopentyl when E is —CH₂—; D is —(CH₂)₂—, —(CH₂)₃—, —C(O)—, phenyl, cyclohexyl or cyclopentyl when Z is NR₄₅ or —O—; D is —(CH₂)₂—, —(CH₂)₃—, phenyl, cyclohexyl or cyclopentyl when Z is —SO₂— or —S—;
X—Y is —C(O)NR₄₆—, —NR₄₇C(O)—, —C(O)O—, —CH₂CH₂—, —CH═CH—, —C≡C—, —NR₄₈CH₂—, —CH₂NR₄₉—, —O—CH₂—, —CH₂—O—, —SO₂NR₅₀—, —NR₅₁SO₂— or cyclopropyl;
A is hydrogen, —OR₅₂, alkyl, substituted alkyl, alkenyl, substituted alkenyl, alkynyl, substituted alkynyl, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl, substituted heteroarylalkynyl or halo;
B is hydrogen, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl, substituted heteroarylalkynyl, halo, —NR₅₃R₅₄, —O—R₅₅, —S—R₅₆ or —SO₂-R₅₇; R₈-R₅₃ and R₅₈-R₆₄ are independently hydrogen, alkyl, substituted alkyl, alkenyl, substituted alkenyl, alkynyl, substituted alkynyl, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl or substituted heteroarylalkynyl;
R₅₄ is alkyl, substituted alkyl, alkenyl, substituted alkenyl, alkynyl, substituted alkynyl, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl, substituted heteroarylalkynyl, —C(O)R₅₈, —C(O)OR₅₉, —C(O)NR₆₀R₆₁, or —SO₂R₆₂;
R₅₅-R₅₇ are alkyl, substituted alkyl, alkenyl, substituted alkenyl, alkynyl, substituted alkynyl, aryl, substituted aryl, arylalkyl, substituted arylalkyl, arylalkenyl, substituted arylalkenyl, arylalkynyl, substituted arylalkynyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroalkyl, substituted heteroalkyl, heteroalkenyl, substituted heteroalkenyl, heteroalkynyl, substituted heteroalkynyl, heteroaryl, substituted heteroaryl, heteroarylalkyl, substituted heteroarylalkyl, heteroarylalkenyl, substituted heteroarylalkenyl, heteroarylalkynyl or substituted heteroarylalkynyl;
R₅ is hydrogen or —F;
R₆ is hydrogen, —F or —OR₅₃; and
R₇ is hydrogen, —F or —OR₆₄;
provided that R₅ is hydrogen when R₄ is —C(O)NR₄₁R₄₂, —C(O)R₄₃, —C(O)OR₄₄ or —CN;
provided that A is not hydrogen, halo or —OR₅₂ when B is hydrogen or halo;
provided that B is not hydrogen or halo when A is hydrogen, halo or —OR₅₂;
provided that R₇ is hydrogen when B is halo, —NR₅₃R₅₄, —O—R₅₅, —S—R₅₆ or —SO₂R₅₇;
provided that R₆ is —OR₆₃ only when X—Y are —CH₂CH₂—, —CH═CH—, —C≡C— or cyclopropyl;
provided that A is not —OR₅₂ when R₆ is —OR₆₃; and
provided that A is not —Cl, —Br or —I when R₆ is —F.

2. The compound of claim 1 of Formula (II):

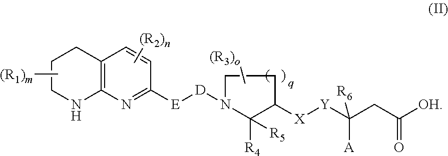

3. The compound of claim 1 of Formula (III):

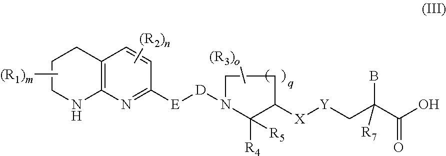

4. The compound of claim 1 of Formula (IV):

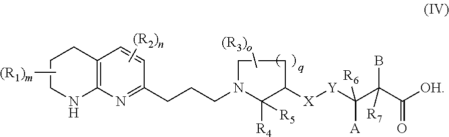

5. The compound of claim 1 of Formula (V):

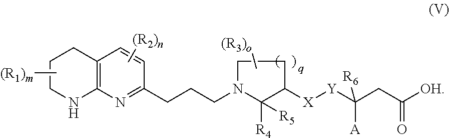

6. The compound of claim 1 of Formula (VI):

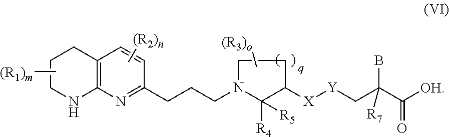

7. The compound of any one of claims 1-6, wherein each R₁ is independently alkyl, substituted alkyl, alkenyl, substituted alkenyl, phenyl, substituted phenyl, cycloalkyl, cycloheteroalkyl, heteroalkyl, —F, —C(O)NR₈R₉, —C(O)OR₁₀, —OC(O)OR₁₅, —CF₃, or —OR₁₅.

8. The compound of any one of claims 1-6, wherein each R₁ is independently (C₁-C₄) alkyl, (C₂-C₄) alkenyl, phenyl, substituted phenyl, (C₅-C₇) cycloalkyl, (C₅-C₇)cycloheteroalkyl, —F, or —CF₃.

9. The compound of any one of claims 1-6, wherein m is 0 or 1.

10. The compound of any one of claims 1-6, wherein each R₂ is independently alkyl, substituted alkyl, alkenyl, substituted alkenyl, phenyl, substituted phenyl, cycloalkyl, substituted cycloalkyl, cycloalkenyl, substituted cycloalkenyl, heteroalkyl, cycloheteroalkyl, cycloheteroalkenyl, halo, —C(O)NR₁₉R₂₀, —C(O)OR₂₁, —NR₂₂C(O)OR₂₃, —NR₂₄C(O)OR₂₅, —OC(O)OR₂₆, —CN, —CF₃, —NR₂₇SO₂R is or —OR₂₉.

11. The compound of any one of claims 1-6, wherein each R₂ is independently (C₁-C₄) alkyl, (C₂-C₄) alkenyl, phenyl, substituted phenyl, (C₅-C₇) cycloalkyl, (C₅-C₇) cycloheteroalkyl, halo, C(O)NR₁₉R₂₀, —C(O)OR₂₁, —NR₂₂C(O)OR₂₃, —NR₂₄C(O)OR₂₅, —OC(O)OR₂₆, —CN, —CF₃, —NR₂₇SO₂R₂₈ or —OR₂₉.

12. The compound of any one of claims 1-6, wherein n is 0 or 1.

13. The compound of any one of claims 1-6, wherein each R₃ is independently, alkyl, substituted alkyl, alkenyl, substituted alkenyl, phenyl, substituted phenyl, cycloalkyl, heteroalkyl, cycloheteroalkyl, —F, —C(O)NR₃₀R₃₁, —C(O)OR₃₂, —OC(O)OR₃₇, —CF₃, or —OR₄o.

14. The compound of any one of claims 1-6, wherein each R₃ is independently (C₁-C₄) alkyl, (C₂-C₄) alkenyl, phenyl, substituted phenyl, (C₅-C₇) cycloalkyl, (C₅-C₇) cycloheteroalkyl, —F, or —CF₃.

15. The compound of any one of claims 1-6, wherein o is 0 or 1.

16. The compound of any one of claims 1-6, wherein o is 0, 1, 2 or 3.

17. The compound of any one of claims 1-6, wherein R₄ is hydrogen, alkyl, substituted alkyl, alkenyl, substituted alkenyl, —F, —C(O)NR₄₁R₄₂, —C(O)R₄₃, —C(O)OR₄₄ or —CF₃.

18. The compound of any one of claims 1-6, wherein R₄ is hydrogen, (C₁-C₄) alkyl, (C₂-C₄) alkenyl, —F or —CF₃.

19. The compound of any one of claims 1-6, wherein R₈-R₅₃ and R₅₈-R₆₄ are independently hydrogen, alkyl, alkenyl, aryl, substituted aryl, arylalkyl, substituted arylalkyl, cycloalkyl, substituted cycloalkyl, heteroalkyl, heteroalkenyl, heteroaryl, substituted heteroaryl, cycloheteroalkyl or substituted cycloheteroalkyl.

20. The compound of any one of claims 1-6, wherein R₈-R₅₃ and R₅₈-R₆₄ are independently hydrogen, (C₁-C₄) alkyl, aryl, substituted aryl, cycloalkyl, substituted cycloalkyl, heteroaryl, substituted heteroaryl, cycloheteroalkyl or substituted cycloheteroalkyl.

21. The compound of any one of claims 1-6, wherein each R₁ is independently alkyl, substituted alkyl, alkenyl, substituted alkenyl, phenyl, substituted phenyl, cycloalkyl, heteroalkyl, cycloheteroalkyl, —F, —C(O)NR₈R₉, —C(O)OR₁₀, —OC(O)OR₁₅, —CF₃, or —OR₁₅, m is 0 or 1, each R₂ is independently alkyl, substituted alkyl, alkenyl, substituted alkenyl, phenyl, substituted phenyl, cycloalkyl, substituted cycloalkyl, cycloalkenyl, substituted cycloalkenyl, heteroalkyl, cycloheteroalkyl,
halo, —C(O)NR₁₉R₂₀, —C(O)OR₂₁, —NR₂₂C(O)OR₂₃, —NR₂₄C(O)OR₂₅, —OC(O)OR₂₆, —CN, —CF₃, —NR₂₇SO₂R₂₈ or —OR₂₉, n is 0 or 1, each R₃ is independently, alkyl, substituted alkyl, alkenyl, substituted alkenyl, phenyl, substituted phenyl, cycloalkyl, heteroalkyl, cycloheteroalkyl, —F, —C(O)NR₃₀R₃₁, —C(O)OR₃₂, —OC(O)OR₃₇, —CF₃, or —OR₄₀, o is 0, 1, 2 or 3, R₄ is hydrogen, alkyl, substituted alkyl, alkenyl, substituted alkenyl, —F, —C(O)NR₄₁R₄₂, —C(O)OR₄₃, —C(O)OR₄₄ or —CF₃ and R₈-R₃₃ and R₅₈-R₆₄ are independently hydrogen, alkyl, alkenyl, aryl, substituted aryl, arylalkyl, substituted arylalkyl, cycloalkyl, substituted cycloalkyl, heteroalkyl, heteroalkenyl, heteroaryl, substituted heteroaryl, cycloheteroalkyl or substituted cycloheteroalkyl.

22. The compound of any one of claims 1-6, wherein each R₁ is independently (C₁-C₄) alkyl, (C₂-C₄) alkenyl, phenyl, substituted phenyl, (C₅-C₇) cycloalkyl, (C₅-C₇) cycloheteroalkyl, —F, or —CF₃, m is 0 or 1, each R₂ is independently (C₁-C₄) alkyl, (C₂-C₄) alkenyl, phenyl, substituted phenyl, (C₅-C₇) cycloalkyl, (C₅-C₇) cycloheteroalkyl, halo, C(O)NR₁₉R₂₀, —C(O)OR₂₁, —NR₂₂C(O)OR₂₃, —NR₂₄C(O)OR₂₅, —OC(O)OR₂₆, —CN, —CF₃, —NR₂₇SO₂R₂₈ or —OR₂₉, is 0 or 1, each R₃ is independently (C₁-C₄) alkyl, (C₂-C₄) alkenyl, phenyl, substituted phenyl, (C₅-C₇) cycloalkyl, (C₅-C₇) cycloheteroalkyl, —F, or —CF₃, o is 0 or 1, R₄ is hydrogen, (C₁-C₄) alkyl, (C₂-C₄) alkenyl, —F or —CF₃ and R₈-R₅₃ and R₅₈-R₆₄ are independently hydrogen, (C₁-C₄) alkyl, aryl, substituted aryl, cycloalkyl, substituted cycloalkyl, heteroaryl, substituted heteroaryl, cycloheteroalkyl or substituted cycloheteroalkyl.

23. The compound of any one of claims 1, 2, 4 or 5, wherein A is aryl, substituted aryl, arylalkyl, substituted arylalkyl, arylalkenyl, substituted arylalkenyl, cycloalkyl, substituted cycloalkyl, cycloalkenyl, substituted cycloalkenyl, heteroaryl, substituted heteroaryl, heteroarylalkyl, substituted heteroarylalkyl, heteroarylalkenyl, substituted heteroarylalkenyl, cycloheteroalkyl, substituted cycloheteroalkyl, cycloheteroalkenyl or substituted cycloheteroalkenyl.

24. The compound of any one of claims 1, 2, 4 or 5, wherein A is aryl, substituted aryl, arylalkyl, substituted arylalkyl, heteroaryl, substituted heteroaryl, heteroarylalkyl or substituted heteroarylalkyl.

25. The compound of any one of claims 1, 2, 4 or 5, wherein A is aryl, substituted aryl, heteroaryl or substituted heteroaryl.

26. The compound of any one of claims 1, 3, 4 or 6, wherein B is aryl, substituted aryl, arylalkyl, substituted arylalkyl, arylalkenyl, substituted arylalkenyl, cycloalkyl, substituted cycloalkyl, cycloalkenyl, substituted cycloalkenyl, heteroaryl, substituted heteroaryl, heteroarylalkyl, substituted heteroarylalkyl, heteroarylalkenyl, substituted heteroarylalkenyl, cycloheteroalkyl, substituted cycloheteroalkyl, cycloheteroalkenyl or substituted cycloheteroalkenyl.

27. The compound of any one of claims 1, 3, 4 or 6, wherein B is aryl, substituted aryl, arylalkyl, substituted arylalkyl, heteroaryl, substituted heteroaryl, heteroarylalkyl or substituted heteroarylalkyl.

28. The compound of any one of claims 1, 3, 4 or 6, wherein B is aryl, substituted aryl, heteroaryl or substituted heteroaryl.

29. The compound of any one of claims 1, 3, 4 or 6, wherein B is —NR₅₃R₅₄.

30. The compound of any one of claims 1, 3, 4 or 6, wherein B is —NHR₅₄, R₅₄ is aryl, substituted aryl, arylalkyl, substituted arylalkyl, heteroaryl, substituted heteroaryl, heteroarylalkyl or substituted heteroarylalkyl —C(O)R₅₈, —C(O)OR₅₉, or —SO₂R₆₂.

31. The compound of any one of claims 1, 3, 4 or 6, wherein B is —NHR₅₄ and R₅₄ is aryl, substituted aryl, heteroaryl, substituted heteroaryl, —C(O)R₅₈, —C(O)OR₅₉, or —SO₂R₆₂.

32. The compound of claim 1 of Formula (VI):

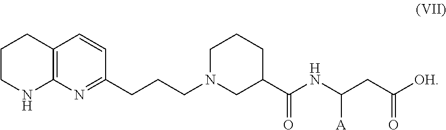

33. The compound of claim 32, wherein A is aryl, substituted aryl, heteroaryl or substituted heteroaryl.

34. The compound of claim 32, wherein A is aryl, substituted phenyl, heteroaryl or substituted heteroaryl.

35. The compound of claim 1 of Formula (VIII):

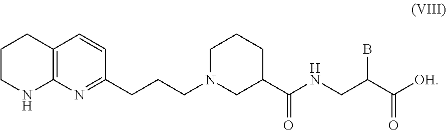

36. The compound of claim 35, wherein B is aryl, substituted aryl, heteroaryl or substituted heteroaryl.

37. The compound of claim 35, wherein B is aryl, substituted phenyl, heteroaryl or substituted heteroaryl.

38. The compound of claim 35, wherein B is —NHR₅₄ and R₅₄ is aryl, substituted aryl, heteroaryl, substituted heteroaryl, —C(O)R₅₈, —C(O)OR₅₉, or —SO₂R₆₂.

39. The compound of claim 1 of Formula (IX):

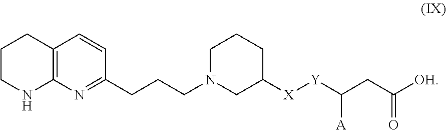

40. The compound of claim 1, wherein A is phenyl or substituted phenyl.

41. A compound of Formula (Ia): 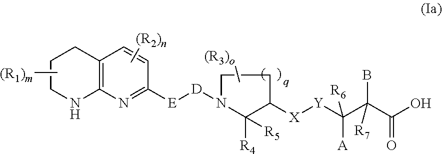
or pharmaceutically acceptable salts thereof, wherein:
q is 1, 2 or 3;
R₁ and R₃ are each independently selected at each occurrence from halogen, C₁₋₄ alkyl, C₁₋₄ haloalkyl, —OR¹¹, —SR¹¹, —N(R¹¹)₂, —C(O)N(R¹¹)₂, —C(O)OR¹¹, ═O, ═S, and —CN;
m is selected from 0, 1, 2, 3, 4, 5, and 6;
o is selected from 0, 1, 2, 3, 4, 5, 6, 7, and 8;
R₂ is independently selected at each occurrence from halogen, C₁₋₄ alkyl, C₁₋₄haloalkyl, —OR₁₂, —SR¹¹, —N(R¹¹)₂, —CN, and —NO₂;
n is 0, 1 or 2;
R₄ and R₅ are each independently selected from hydrogen, halogen, C₁₋₄ alkyl, C₁₋₄haloalkyl, —OR¹³, —SR¹³, —N(R¹³)₂, and —CN; or R₄ and R₅ are taken together to form a double bonded substituent selected from ═O, ═S and ═N(R¹³);
D is selected from a bond, —C(O)—, —C≡CCH₂—, and —CH═CHCH₂—;
E is selected from C₁₋₄ alkylene and —(CH₂)Z—,
wherein Z is selected from —NH—, —S—, —SO₂—, and —O—;
X—Y is selected from: ^{λ}—C(O)N(R¹⁴)—, ^{λ}—N(R₁₄)C(O)—, ^{λ}—N(R¹⁴)C(O)C(R¹⁴)₂—, ^{λ}—C(O)O—, ^{λ}—C(R¹⁵)₂C(R¹⁵)₂—, ^{λ}—CH═CH—, ^{λ}—C≡C—, ^{λ}—N(R¹⁴)C(R¹⁵)₂—, ^{λ}—C(R¹⁵)₂N(R¹⁴)—, ^{λ}—O—, ^{λ}—OC(R¹⁵)₂—, ^{λ}—C(R¹⁵)₂—, ^{λ}—SO₂N(R¹⁴)—, and ^{λ}—N(R₁₄)SO₂—;
wherein denotes the attachment of X—Y to 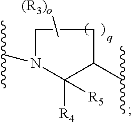
R₆ and R₇ are each independently selected at each occurrence from:
hydrogen, halogen, C₁₋₄alkyl, C₁₋₄haloalkyl, —OR¹⁶, and —CN;
A is selected from (i) and (ii):
(i) hydrogen, halogen, and —CN, or A and R⁶ come together to form a C₃₋₆ carbocycle or 3- to 6-membered heterocycle;
(ii) —OR¹⁷, —SR¹⁷, —N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —C(O)R¹⁷, —C(O)OR¹⁷, —OC(O)R¹⁷, —OC(O)N(R¹⁷)₂, —C(O)N(R¹⁷)₂, —S(O)R¹⁷, —S(O)₂R¹⁷, and —S(O)₂N(R¹⁷)₂;
C₁₋₆ alkyl optionally substituted with one or more substituents independently selected from:
halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═N(R¹⁷), —N₃, and —CN, C₃₋₁₀ carbocycle, and 3- to 10-membered heterocycle,
wherein the C₃₋₁₀ carbocycle and 3- to 10-membered heterocycle are each optionally substituted with one or more substituents independently selected from halogen, C₁₋₆ alkyl, C₁₋₄ haloalkyl, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═N(R¹⁷), —N₃, and —CN; and
C₃₋₁₂ carbocycle and 3- to 12-membered heterocycle, any of which is optionally substituted with one or more substituents independently selected from:
halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═N(R¹⁷), —N₃, and —CN,
C₁₋₆alkyl optionally substituted with one or more substituents independently selected from halogen, —OR¹⁷, —SR¹⁷, —N(R¹⁷)₂, —C(O)R¹⁷, —C(O)OR¹⁷, —OC(O)R¹⁸, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)C(S)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, ═S, ═N(R¹⁷), —N₃, and —CN; and
C₃₋₁₀ carbocycle and 3- to 10-membered heterocycle, any of which is optionally substituted with one or more substituents independently selected from halogen, C₁₋₄ alkyl, C₁₋₄haloalkyl, and ═O:
B is selected from (I) when A is selected from (ii), or
B is selected from (II) when A is selected from (i):
(I) hydrogen, halogen, and —CN, or B and R¹⁷ are taken together to form a C₃₋₆ carbocycle or a 3- to 6-membered heterocycle;
(II) —OR¹⁸, —SR¹⁸, —N(R¹⁸)₂, —C(O)R¹⁸, —C(O)OR¹⁸, —OC(O)R¹³, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)R¹⁸, and —S(O)N(R¹⁸)₂;
C₁₋₆ alkyl optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═N(R¹⁸), —N₃, —CN, C₃₋₁₀ carbocycle and 3- to 10-membered heterocycle,
wherein the C₃₋₁₀ carbocycle and 3- to 10-membered heterocycle are optionally substituted with one or more substituents independently selected from halogen, C₁₋₆ alkyl, C₁₋₆ haloalkyl, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═N(R¹⁸), —N₃, and —CN; and
C₃₋₁₂ carbocycle and 3- to 12-membered heterocycle, any of which is optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═N(R¹⁸), —N₃, and —CN;
C₁₋₆ alkyl optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═N(R¹⁸), —N₃, —CN, C₃₋₆ carbocycle and 3- to 6-membered heterocycle,
wherein the C₃₋₆ carbocycle and 3- to 6-membered heterocycle are each optionally substituted with one or more substituents independently selected from halogen, C₁₋₄ alkyl, C₁₋₄ haloalkyl, and ═O; and
C₃₋₁₀ carbocycle and 3- to 10-membered heterocycle, any of which is optionally substituted with one or more substituents independently selected from halogen, C₁₋₄-alkyl, C₁₋₄ haloalkyl, and ═O;
R¹¹, R¹², R¹³, R¹⁴, and R¹⁶ are each independently selected at each occurrence from hydrogen, C₁₋₄ alkyl, and C₁₋₄ haloalkyl;
R¹⁵ is independently selected at each occurrence from hydrogen, halogen, C₁₋₄ alkyl, and C₁₋₄ haloalkyl;
R¹⁷ is independently selected at each occurrence from:
hydrogen;
C₁₋₆ alkyl optionally substituted with one or more substituents independently selected from: halogen, —OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —NO₂, ═O, ═S, ═N(R²¹), —N₃, and —CN; and
C₃₋₆ carbocycle and 3- to 6-membered heterocycle, any of which are optionally substituted with one or more substituents independently selected from: halogen, C₁₋₄ alkyl, C₁₋₄haloalkyl, —OR²¹, —SR²¹, —N(R²¹)₂, —C(O)R²¹, —C(O)OR²¹, —OC(O)R²¹, —OC(O)N(R²¹)₂, —C(O)N(R²¹)₂, —N(R²¹)C(O)R²¹, —N(R²¹)C(O)OR²¹, —N(R²¹)C(O)N(R²¹)₂, —N(R²¹)C(S)N(R²¹)₂, —N(R²¹)S(O)₂(R²¹), —S(O)R²¹, —S(O)₂R²¹, —S(O)₂N(R²¹)₂, —NO₂, ═O, ═S, ═N(R²¹), —N₃, and —CN;
R¹⁸ is independently selected at each occurrence from:
hydrogen;
C₁₋₆ alkyl optionally substituted with one or more substituents independently selected from:
halogen, —OR²², —SR²², —N(R²²)₂, —C(O)R²², —C(O)OR²², —OC(O)R²², —OC(O)N(R²)₂, —C(O)N(R²²)₂, —N(R²²)C(O)R²², —NO₂, ═O, ═S, ═N(R²²), —N₃, —CN, C₃₋₁₀ carbocycle, and 3- to 10-membered heterocycle,
wherein the C₃₋₁₀ carbocycle and 3- to 10-membered heterocycle each are optionally substituted with one or more substituents independently selected from halogen, C₁₋₆ alkyl, C₁₋₆ haloalkyl, —OR²², —SR²², and —N(R²²)₂; and
C₃₋₁₀ carbocycle and 3- to 10-membered heterocycle, any of which is optionally substituted with one or more substituents independently selected from:
halogen, C₁₋₆ alkyl, C₁₋₆haloalkyl, —OR²², —SR²², —N(R²²)₂, —C(O)R²², —C(O)OR²², —OC(O)R²², —OC(O)N(R²²)₂, —C(O)N(R²²)₂, —N(R²²)C(O)R²², —N(R²²)C(O)OR²², —N(R²²)C(O)N(R²²)₂, —N(R²²)C(S)N(R²²)₂, —N(R²²)S(O)₂(R²²), —S(O)R²², —S(O)₂R²², —S(O)₂N(R²²)₂, —NO₂, ═O, ═S, ═N(R²²), —N₃, —CN, C₃₋₆ carbocycle, and 3- to 6-membered heterocycle;
wherein the C₃₋₆ carbocycle and 3- to 6-membered heterocycle are each optionally substituted with one or more substituents independently selected from halogen, C₁₋₄ alkyl, and C₁₋₄ haloalkyl;
R²¹ and R²² are each independently selected at each occurrence from:
hydrogen;
C₁₋₄ alkyl optionally substituted with one or more substituents independently selected from halogen, hydroxyl, C₃₋₆ carbocycle, and 3- to 6-membered heterocycle, wherein each C₃₋₆ carbocycle and 3- to 6-membered heterocycle is optionally substituted with one or more substituents independently selected from C₁₋₄ alkyl, —N(R²³)₂, and —C(O)N(R²³)₂; and
C₃₋₆ carbocycle and 3- to 12-membered heterocycle, any of which is optionally substituted with one or more substituents independently selected from halogen, C₁₋₄ alkyl, C₁₋₄haloalkyl, C₁₋₄ alkoxy, and ═O; and
R²³ is independently selected at each occurrence from hydrogen and C₁₋₄ alkyl.

42. The compound or salt of claim 41, wherein R³ is selected at each occurrence from halogen, C₁₋₄ alkyl, and —C(O)N(R¹¹)₂; and o is 0, 1, or 2.

43. The compound or salt of claim 41 or 42, wherein q is 1 or 2.

44. The compound or salt of any one of claims 41 to 43, wherein m is 0 and n is 0.

45. The compound or salt of any one of claims 41 to 44, wherein R⁴ and R⁵ are each hydrogen.

46. The compound or salt of any one of claims 41 to 45, wherein D is a bond or —C(O)—.

47. The compound or salt of any one of claims 41 to 46, wherein E is selected from C₁₋₄ alkylene.

48. The compound or salt of any one of claims 41 to 47, wherein X—Y is selected from ^{λ}—C(O)N(R¹⁴)—, ^{λ}—N(R¹⁴)C(O)—, ^{λ}—N(R¹⁴)C(O)CH₂, ^{λ}—CH₂CH₂, ^{λ}—N(R¹⁴)CH₂—, ^{λ}—CH₂N(R¹⁴)—, ^{λ}—O—, —OCH₂—, and ^{λ}—CH₂O—; and R¹⁴ is selected at each occurrence from hydrogen and C₁₋₄ alkyl.

49. The compound or salt of claim 48, wherein X—Y is selected from ^{λ}—C(O)N(R¹⁴)—, ^{λ}—N(R¹⁴)C(O)—, and ^{λ}—OCH₂—; and R¹⁴ is hydrogen.

50. The compound or salt of any one of claims 41 to 49, wherein R¹¹, R¹², R¹³, R¹⁴, R¹⁵, and R¹⁶ are each selected at each occurrence from hydrogen.

51. The compound or salt of any one of claims 41 to 50, wherein R⁶ is selected from hydrogen and —OH, or A and R⁶ are taken together to form a C₃₋₆ carbocycle; and R⁷ is selected from hydrogen and halogen.

52. The compound or salt of any one of claims 41 to 51, wherein R⁶ and R⁷ are each selected from hydrogen.

53. The compound or salt of any one of claims 41 to 52, wherein A is selected from hydrogen, halogen, and —CN, or A and R⁶ are taken together to form a C₃₋₆ carbocycle.

54. The compound or salt of claim 53, wherein A is hydrogen.

55. The compound or salt of any one of claims 41 to 52, wherein B is selected from hydrogen, halogen, and —CN.

56. The compound or salt of claim 55, wherein B is selected from hydrogen and halogen.

57. The compound or salt of any one of claims 41 to 50 and 55 to 56, wherein A is selected from C₃₋₁₂ carbocycle and 3- to 12-membered heterocycle, any of which is optionally substituted with one or more substituents.

58. The compound or salt of claim 57, wherein the C₃₋₁₂ carbocycle and 3- to 12-membered heterocycle of A are selected from phenyl; pyridine; indane; chromane; benzodioxole; 2,3-dihydrobenzofuran: quinoline; 1,2,3,4-tetrahydronaphthalene; naphthalene; quinoxaline; 2′,3′-dihydrospiro[cyclopropane-1,1′-indene]; and pyrazole, any of which is optionally substituted with one or more substituents.

59. The compound or salt of claim 57 or claim 58, wherein the one or more optional substituents on A are selected from:
halogen, —OR¹⁷, N(R¹⁷)₂, —C(O)R¹⁷, —N(R¹⁷)C(O)R¹⁷, —N(R¹⁷)S(O)₂(R¹⁷), ═O, ═S, and —CN;
C₁₋₆ alkyl optionally substituted with one or more substituents independently selected from halogen, —OR¹⁷, —N(R¹⁷)₂, —N(R¹⁷)C(O)R¹⁷, —N(R¹⁷)S(O)₂(R¹⁷), —S(O)R¹⁷, ═O, and —CN; and
C₃₋₁₀ carbocycle and 3- to 10-membered heterocycle, any of which is optionally substituted with one or more substituents independently selected from halogen, C₁₋₄ alkyl, C₁₋₄ haloalkyl, and ═O.

60. The compound or salt of claim 59, wherein the one or more optional substituents on A are selected from: halogen, —OR¹⁷, C₁₋₆ alkyl, C₁₋₆ haloalkyl, C₃₋₁₀ carbocycle, and 3- to 10-membered heterocycle, wherein the C₃₋₁₀ carbocycle, and 3- to 10-membered heterocycle are each optionally substituted with one or more substituents independently selected from C₁₋₄ alkyl, C₁₋₄ haloalkyl, and ═O.

61. The compound or salt of any one of claims 41 to 52 and 55 to 60, wherein R¹⁷ is independently selected at each occurrence from hydrogen, C₁₋₆ alkyl, C₃₋₆ carbocycle and 3- to 6-membered heterocycle.

62. The compound or salt of any one of claims 41 to 52 and 57 to 61, wherein the one or more optional substituents on A is selected from: halogen, hydroxyl, methoxy, trifluoromethyl, propyl, cyclopropyl, cyclopentyl, phenyl, phenoxy,

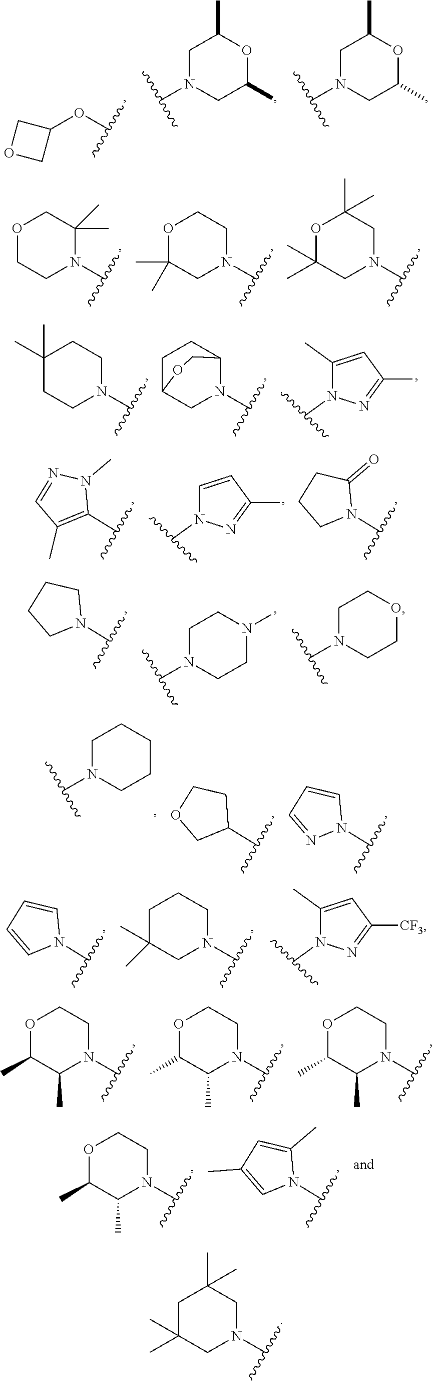

63. The compound or salt of any one of claims 41 to 52 and 55 to 56, wherein A is selected from:

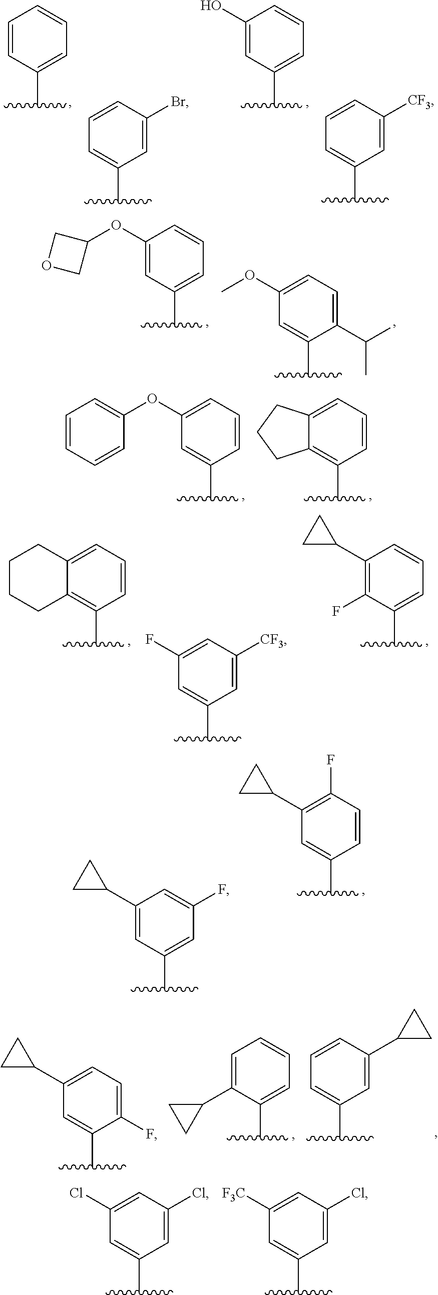

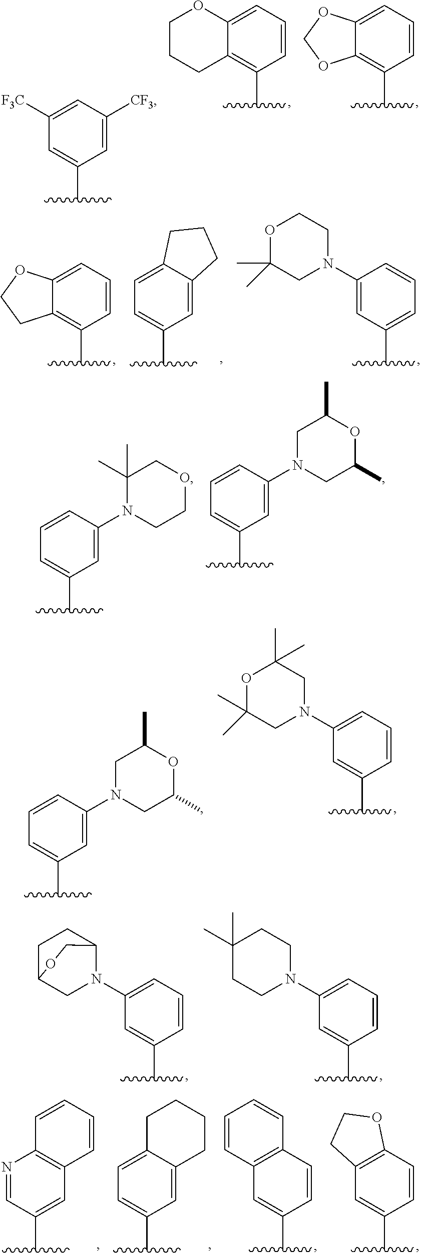

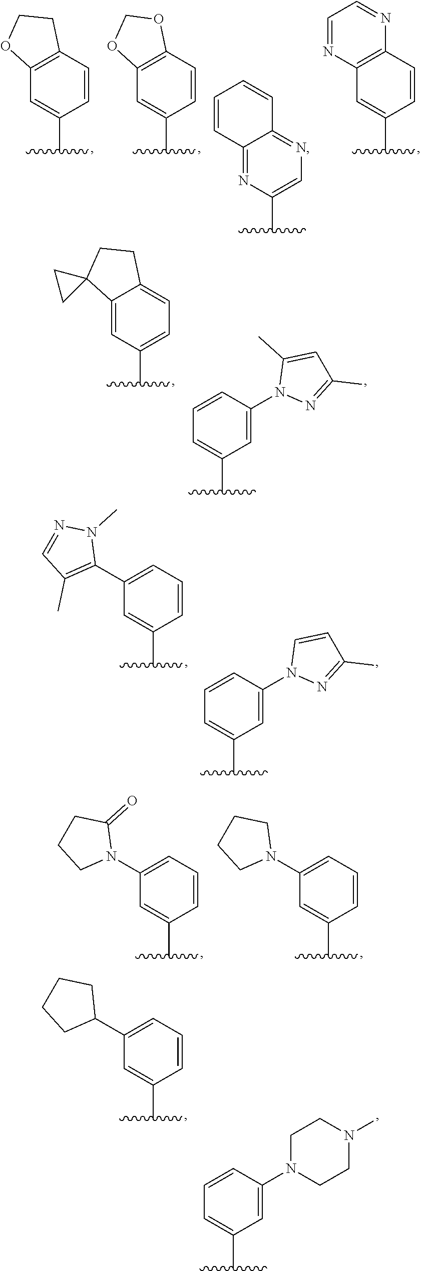

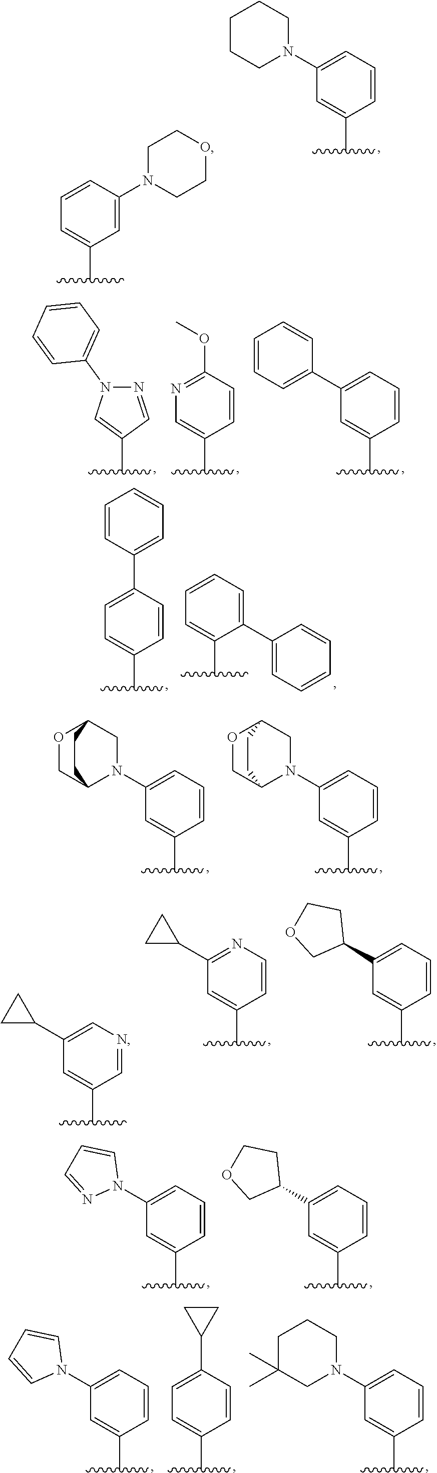

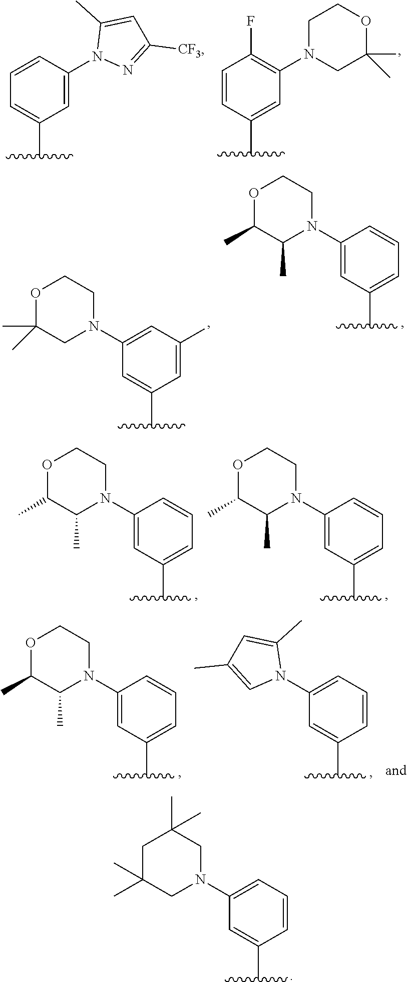

64. The compound or salt of any one of claims 41 to 54, wherein B is selected from:
—OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, and —S(O)₂N(R¹⁸)₂;
C₃₋₁₂ carbocycle, and 3- to 12-membered heterocycle, any of which is optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, ═N(R¹⁸), —N₃, and —CN;
C₁₋₆ alkyl optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, —S(O)₂N(R¹⁸)₂, —NO₂, ═O, ═S, —N₃, —CN, C₃₋₆ carbocycle, and 3- to 6-membered heterocycle,
wherein the C₃₋₆ carbocycle and 3- to 6-membered heterocycle are each optionally substituted with one or more substituents independently selected from halogen, C₁₋₄ alkyl, C₁₋₄ haloalkyl, and ═O; and
C₃₋₁₀ carbocycle and 3- to 10-membered heterocycle, any of which is optionally substituted with one or more substituents independently selected from halogen, C₁₋₄alkyl, C₁₋₄ haloalkyl, and ═O.

65. The compound or salt of claim 64, wherein B is selected from —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)C(S)N(R¹⁸)₂, —N(R¹⁸)S(O)₂(R¹⁸), —S(O)R¹⁸, —S(O)₂R¹⁸, and —S(O)₂N(R¹⁸)₂.

66. The compound or salt of claim 65, wherein B is selected from —N(R¹⁸)₂, —N(R¹⁸)C(O)R¹⁸, —N(R¹⁸)C(O)OR¹⁸, —N(R¹⁸)C(O)N(R¹⁸)₂, and —N(R¹⁸)S(O)₂(R¹⁸).

67. The compound or salt of any one of claims 64 to 66, wherein R¹⁸ is independently selected at each occurrence from hydrogen, C₁₋₆ alkyl, C₃₋₁₀ carbocycle, and 3- to 10-membered heterocycle, wherein the C₁₋₆ alkyl, C₃₋₁₀ carbocycle, and 3- to 10-membered heterocycle are each optionally substituted with one or more substituents.

68. The compound or salt of any one of claims 64 to 67, wherein the C₃₋₁₀ carbocycle and 3- to 10-membered heterocycle of R¹⁸ is independently selected at each occurrence from pyrrolidine, piperidine, phenyl, indoline, bicyclo[2.2.2]octane, cyclohexane, tetrahydropyran, pyridine, oxadiazole, pyrimidine, quinazoline, naphthalene, quinoline, thieno[3,2-d]pyrimidine, thieno[2,3-d]pyrimidine, benzothiazole, indane, thieno[2,3-d]pyrimidine oxide, and cyclopropyl, any of which is optionally substituted with one or more substituents.

69. The compound or salt of any one of claims 64 to 68, wherein the C₃₋₁₀ carbocycle and 3- to 10-membered heterocycle of R¹⁸ are each optionally substituted at each occurrence with one or more substituents independently selected from: halogen, C₁₋₆ alkyl, C₁₋₆haloalkyl, —OR²², —N(R²²)₂, —C(O)R²², —C(O)N(R²²)₂, —N(R²²)C(O)R²², —S(O)₂R²², ═O, —CN, C₃₋₆ carbocycle, and 3- to 6-membered heterocycle, wherein the C₃₋₆ carbocycle and 3- to 6-membered heterocycle are each optionally substituted with one or more substituents independently selected from halogen, C₁₋₄ alkyl, and C₁₋₄haloalkyl.

70. The compound or salt of claim 69, wherein the C₃₋₁₀ carbocycle and 3- to 10-membered heterocycle of R¹⁸ are each optionally substituted at each occurrence with one or more substituents independently selected from halogen, C₁₋₆ alkyl, C₁₋₆ haloalkyl, —N(R²²)₂, —C(O)R²², —C(O)N(R²²)₂, —S(O)₂R²², ═O, C₃₋₆ carbocycle, and 3- to 6-membered heterocycle, wherein the C₃₋₆ carbocycle and 3- to 6-membered heterocycle are each optionally substituted with one or more substituents independently selected from halogen and C₁₋₄ haloalkyl.

71. The compound or salt of any one of claims 64 to 70, wherein R²² is independently selected at each occurrence from hydrogen, C₁₋₄ alkyl, C₃₋₆ carbocycle, and 3- to 6-membered heterocycle, wherein the C₃₋₆ carbocycle and 3- to 6-membered heterocycle are each optionally substituted with one or more substituents independently selected from C₁₋₄ alkyl and C₁₋₄ alkoxy.

72. The compound or salt of any one of claims 64 to 71, wherein the C₃₋₁₀ carbocycle and 3- to 10-membered heterocycle of R¹⁸ are each optionally substituted at each occurrence with one or more substituents independently selected from: halogen, methyl, trifluoromethyl, cyclopropyl,
phenyl, —NH₂, ═O,

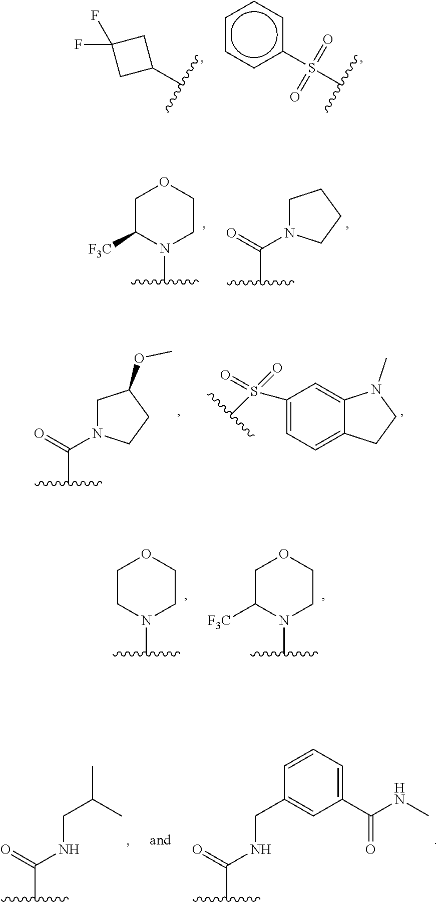

73. The compound or salt of any one of claims 41 to 54, wherein B is selected from:

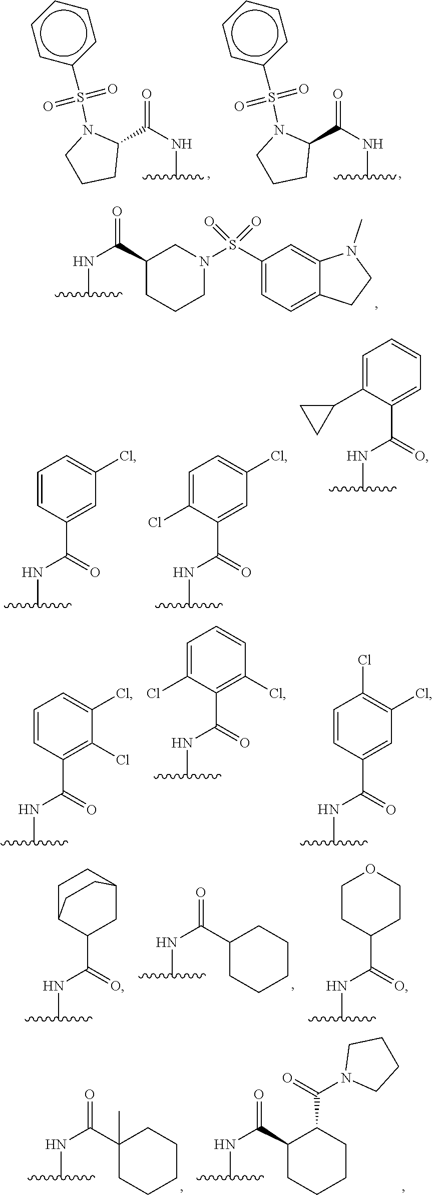

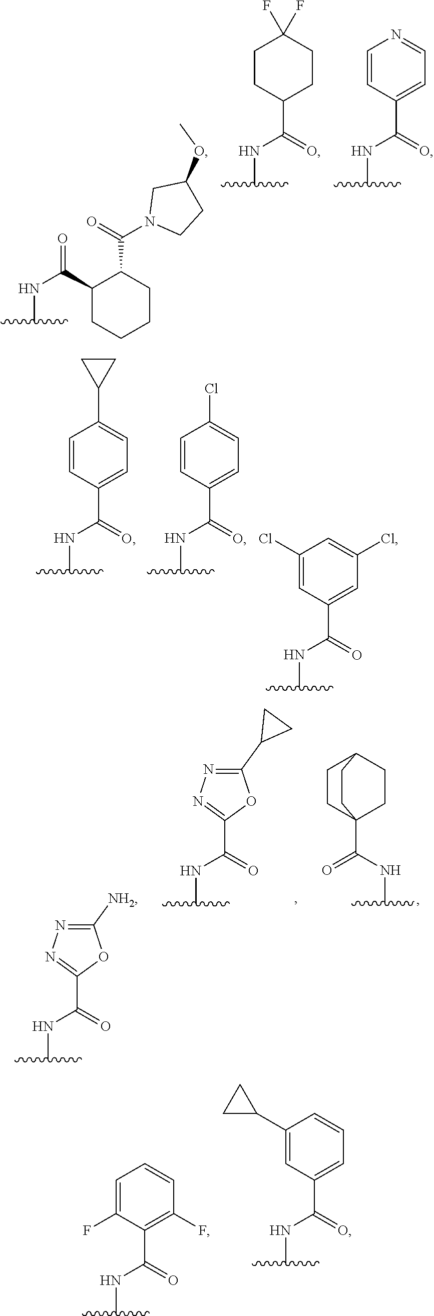

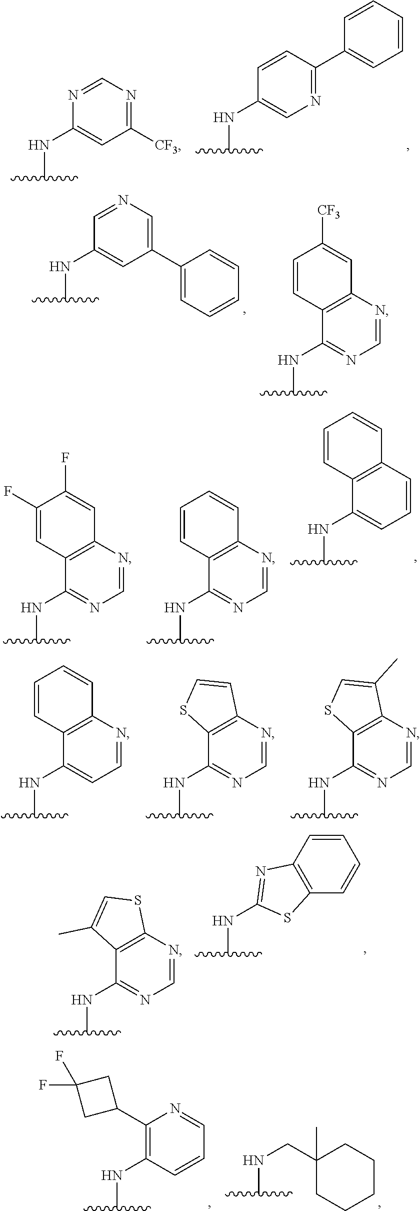

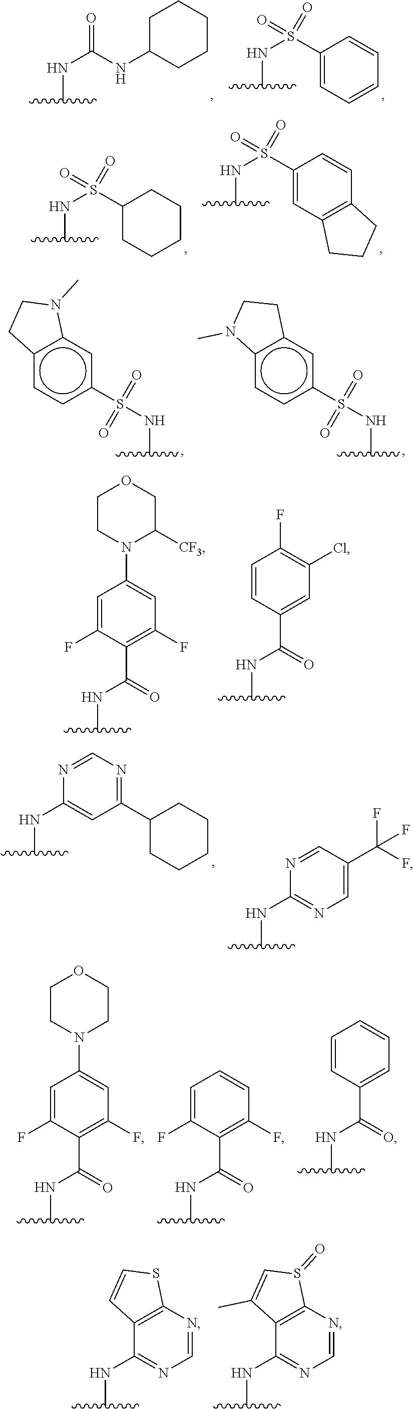

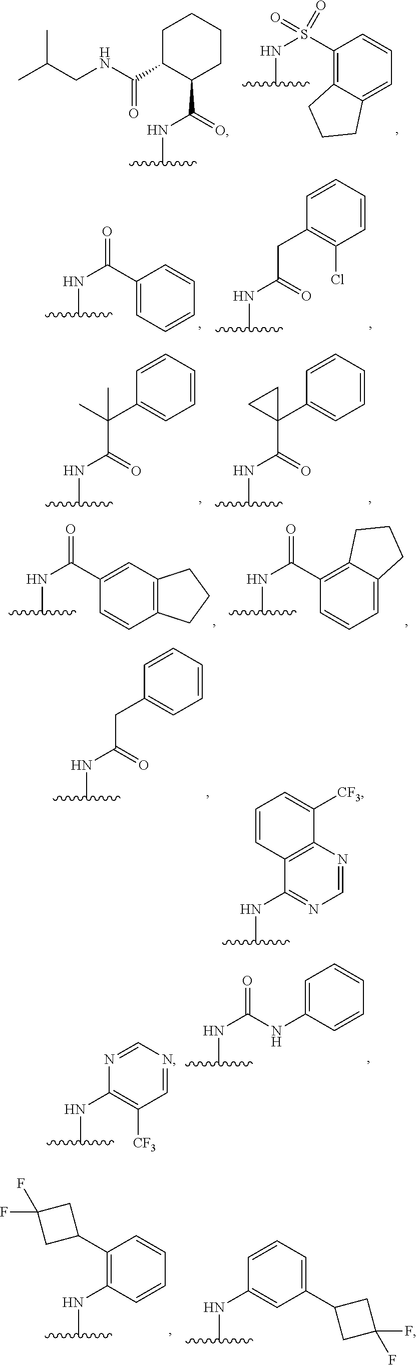

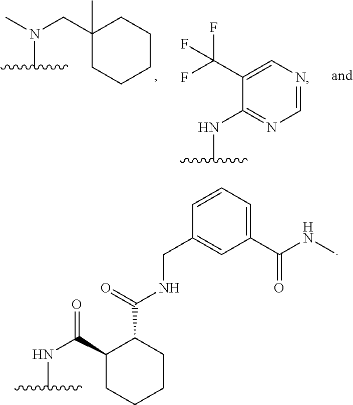

74. The compound or salt of claim 64, wherein B is selected from C₃₋₁₂ carbocycle and 3- to 12-membered heterocycle, any of which is optionally substituted with one or more substituents.

75. The compound or salt of claim 74, wherein the C₃₋₁₂ carbocycle and 3- to 12-membered heterocycle of B is selected from phenyl; pyridinyl, naphthyl; 1,2,3,4-tetrahydronaphthalene; indane; 7-azaindole: indazole; and chromane: any of which is optionally substituted with one or more substituents.

76. The compound or salt of any of claims 74 to 75, wherein the C₃₋₁₂ carbocycle and 3- to 12-membered heterocycle of B are each optionally substituted with one or more substituents independently selected from:
halogen, —OR¹⁸, —N(R¹⁸)₂, —C(O)R¹⁸, ═O, and —CN;
C₁₋₄ alkyl optionally substituted with one or more substituents independently selected from halogen, —OR¹⁸, —SR¹⁸, —N(R¹⁸)₂, —C(O)R¹⁸, ═O, —CN, C₃₋₆ carbocycle, and 3- to 6-membered heterocycle, wherein the C₃₋₆ carbocycle and 3- to 6-membered heterocycle are each optionally substituted with one or more substituents independently selected from halogen, C₁₋₄ alkyl, C₁₋₄ haloalkyl, and ═O; and
C₃₋₁₀ carbocycle and 3- to 10-membered heterocycle, any of which is optionally substituted with one or more substituents independently selected from halogen, C₁₋₄ alkyl, C₁₋₄ haloalkyl, and ═O.

77. The compound or salt of claim 76, wherein the C₃₋₁₂ carbocycle and 3- to 12-membered heterocycle of B are each optionally substituted with one or more substituents independently selected from:
halogen and —OR¹⁸;
C₁₋₆ alkyl optionally substituted with one or more substituents independently selected from halogen, OR¹⁸, and C₃₋₆ carbocycle; and
C₃₋₁₀ carbocycle and 3- to 10-membered heterocycle, wherein the C₃₋₁₀ carbocycle and 3- to 10-membered heterocycle are each optionally substituted with one or more substituents independently selected from halogen and C₁₋₄ alkyl.

78. The compound or salt of any of claims 74 to 77, wherein the C₃₋₁₂ carbocycle and 3- to 12-membered heterocycle of B are each optionally substituted with one or more substituents independently selected from halogen, trifluoromethyl, cyclopropyl, phenyl,

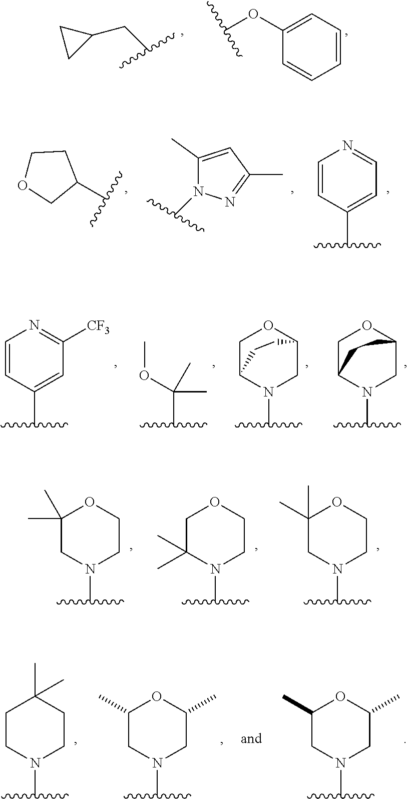

79. The compound or salt of any of claims 51 to 54, wherein B is selected from:

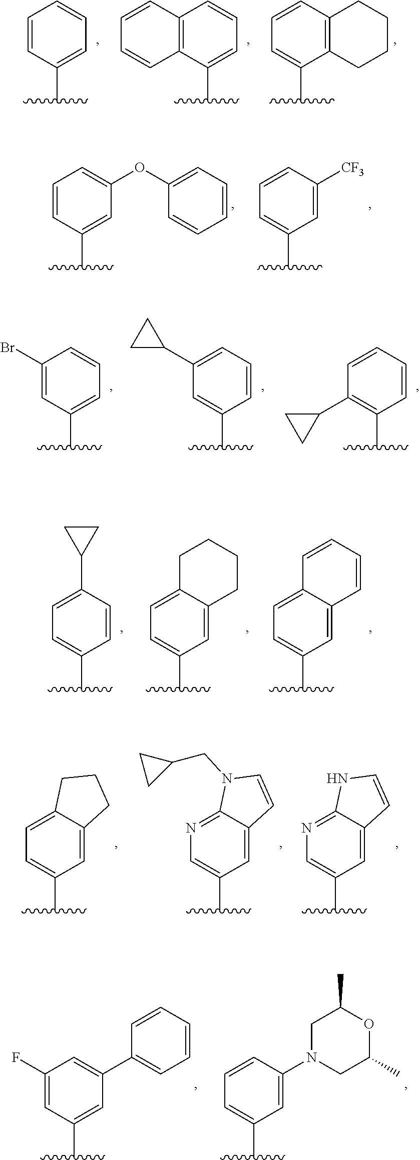

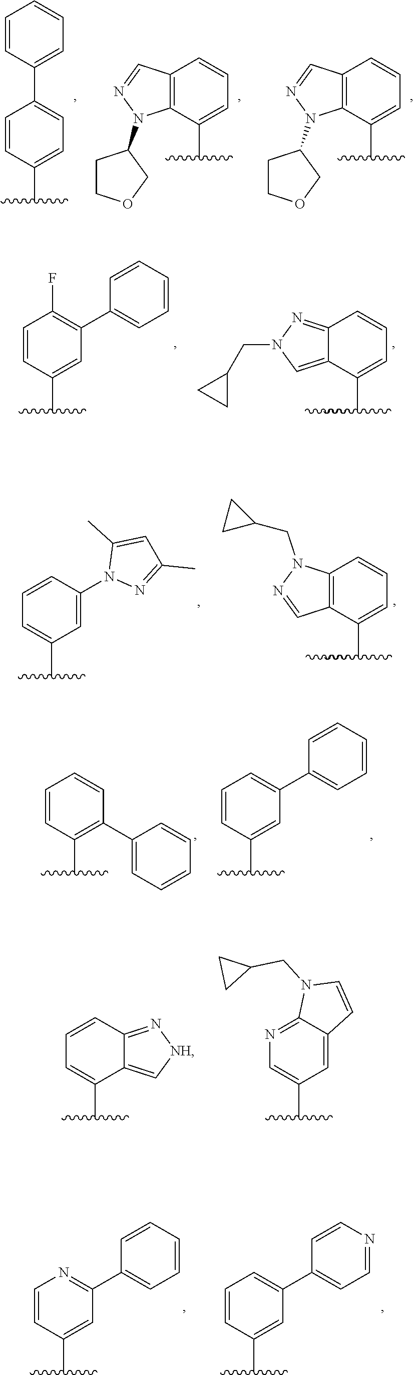

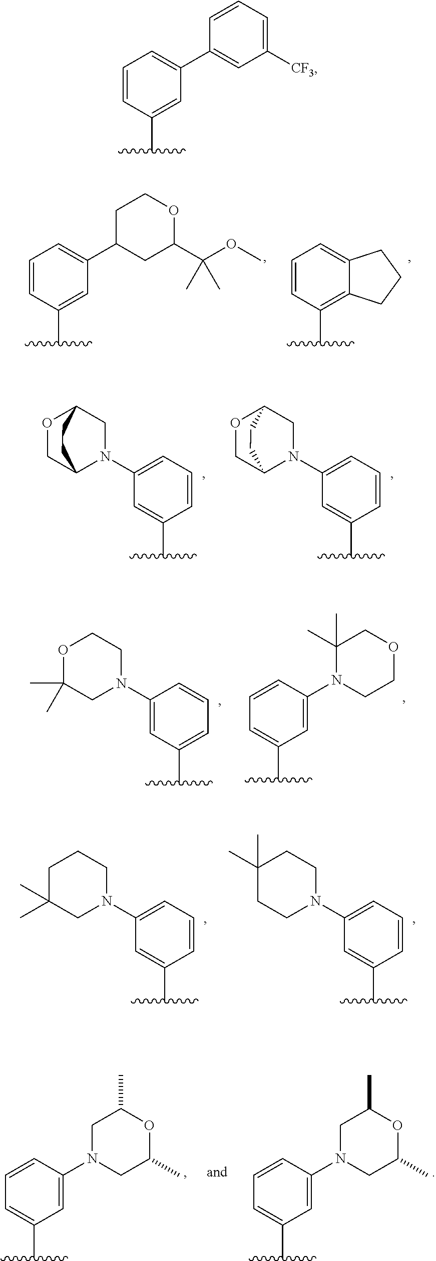

80. The compound or salt of claim 41, wherein the compound of Formula (Ia) is selected from: 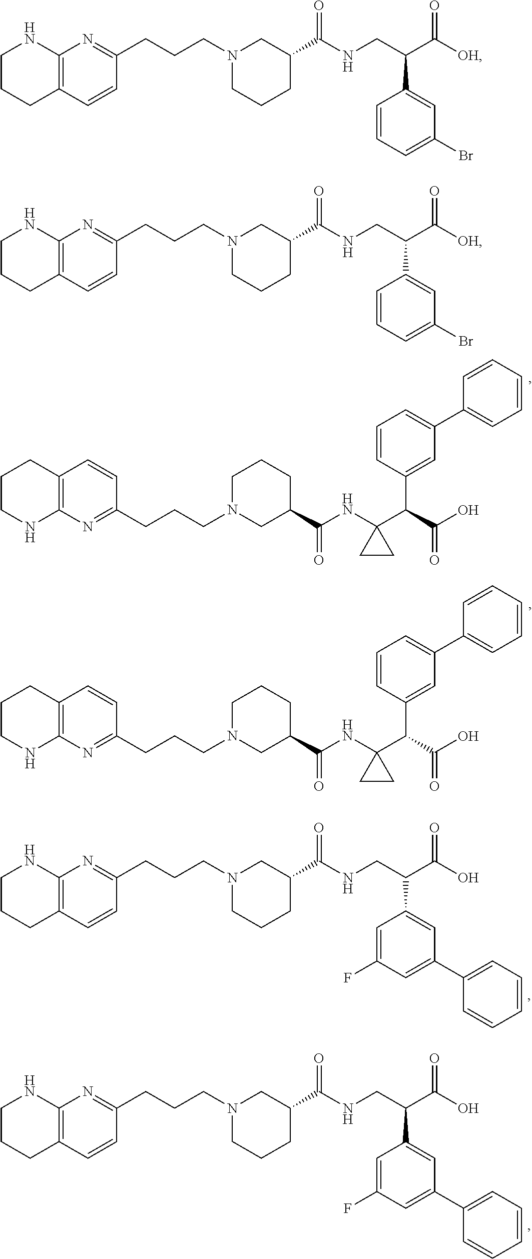 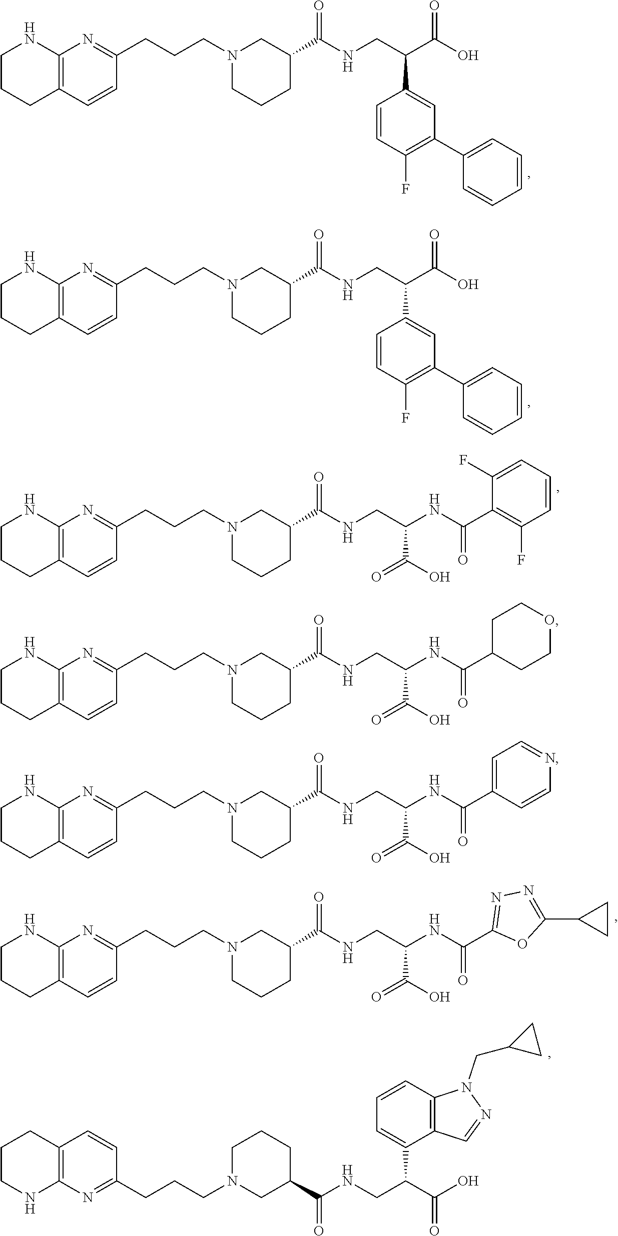 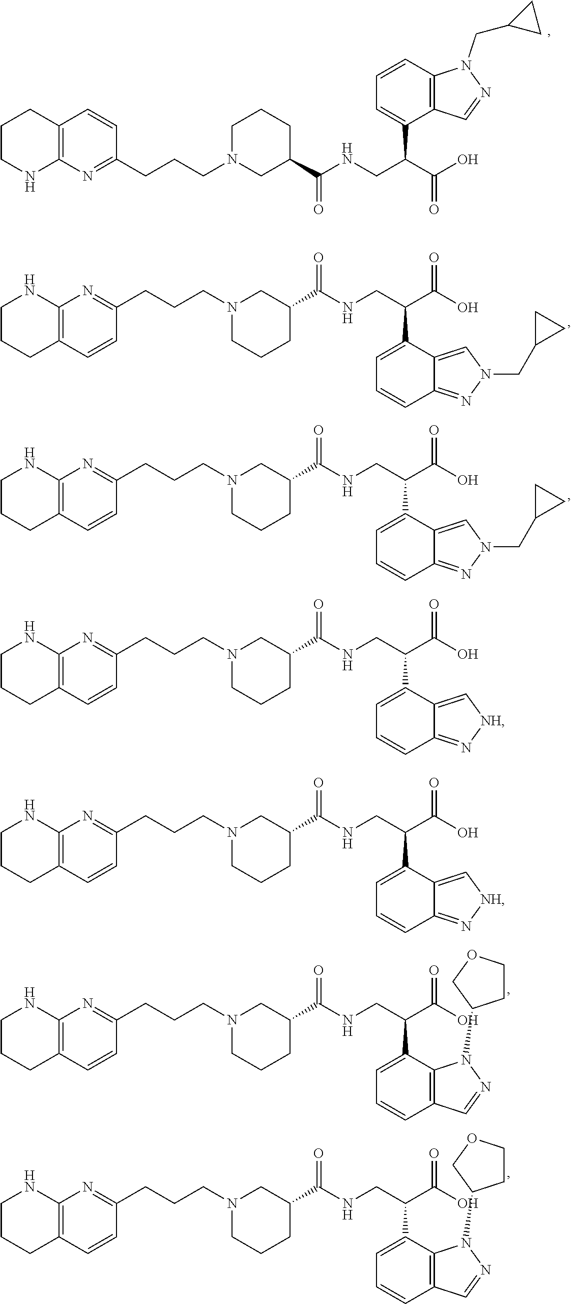 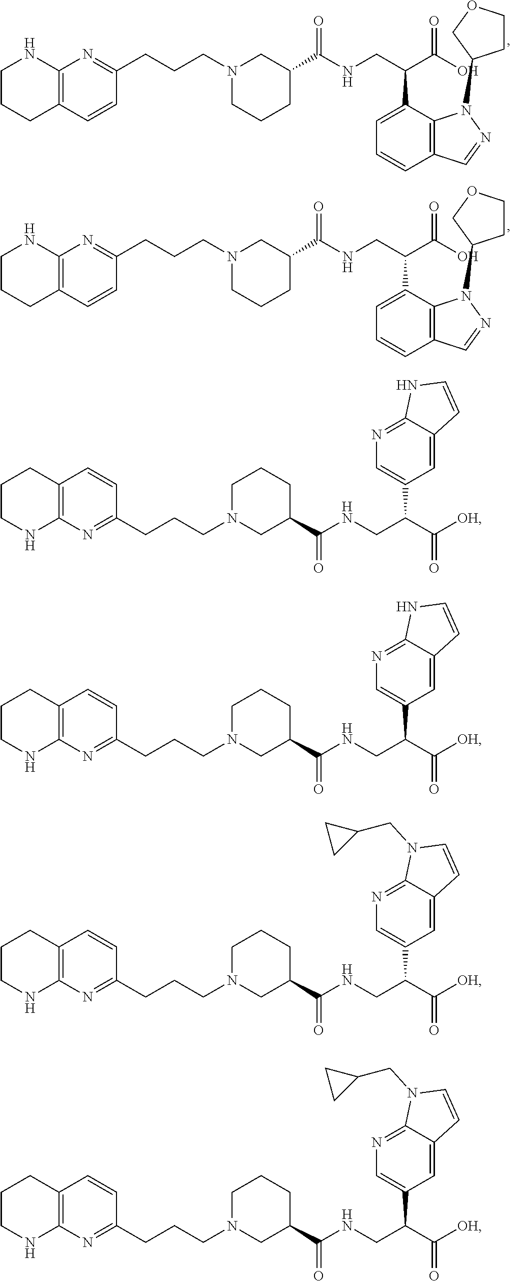 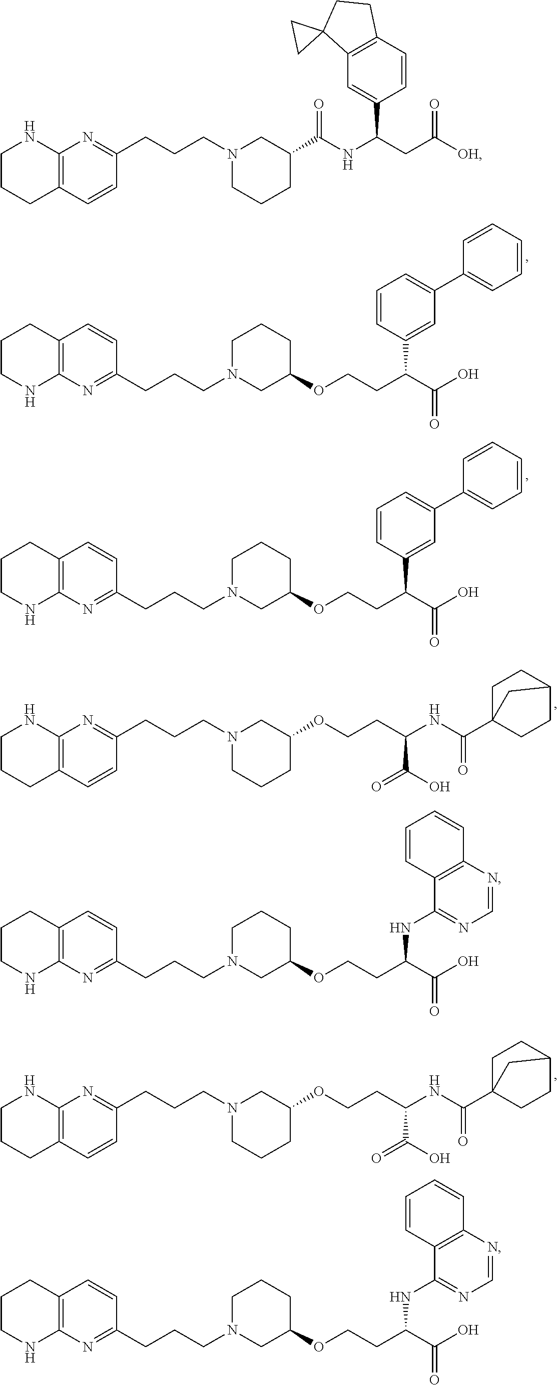 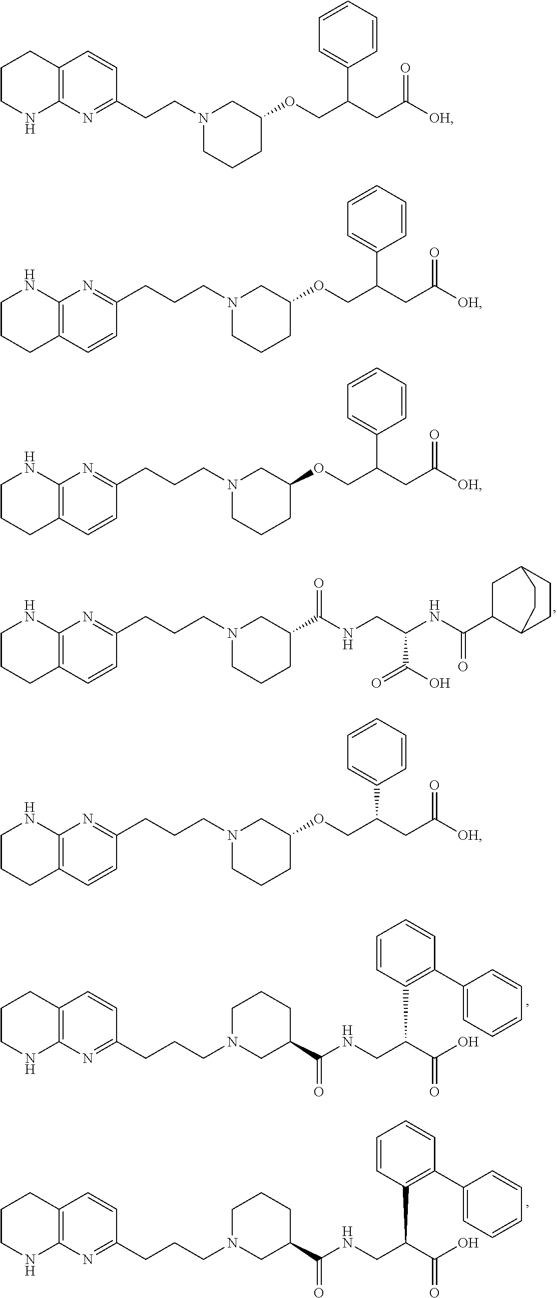 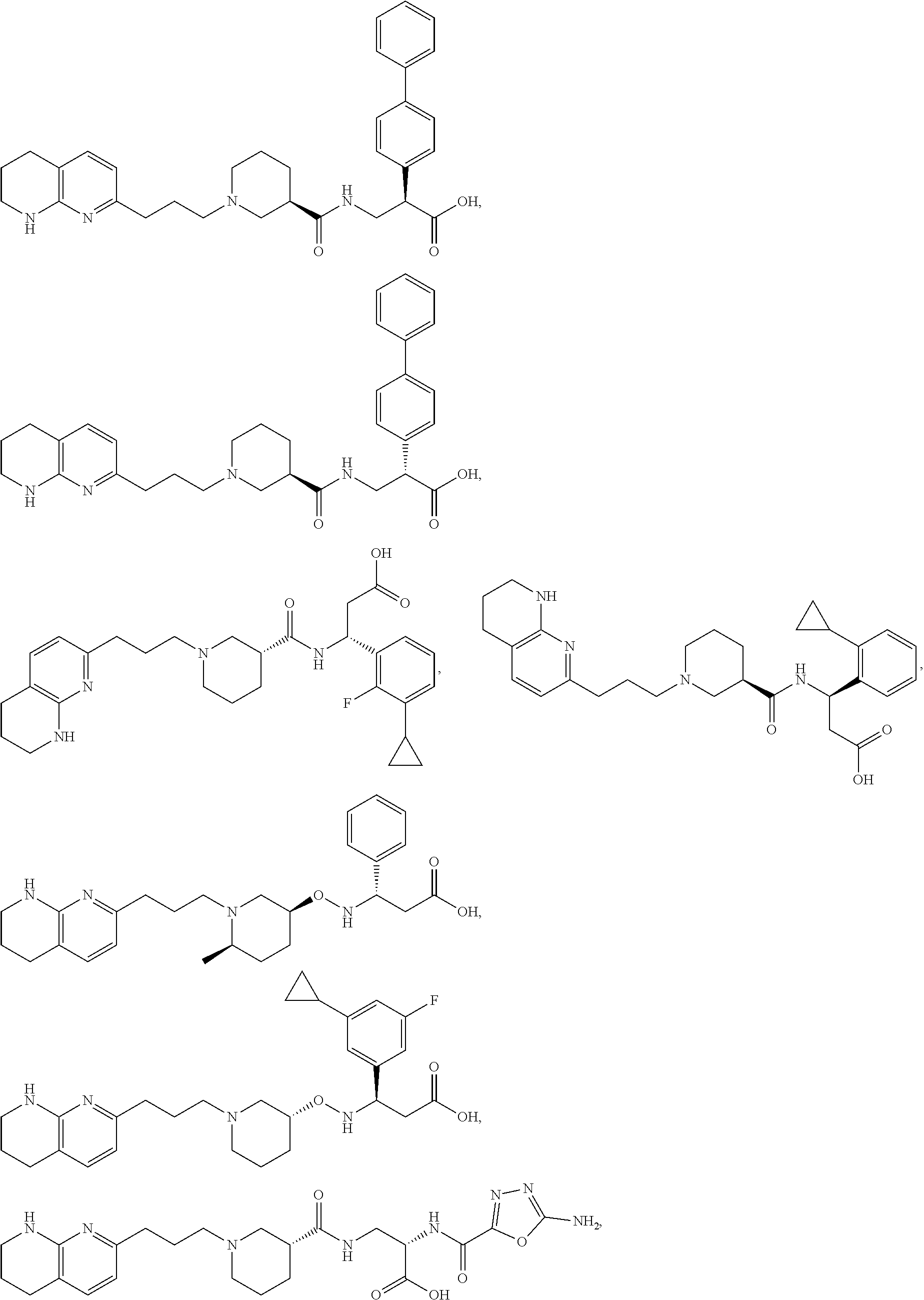 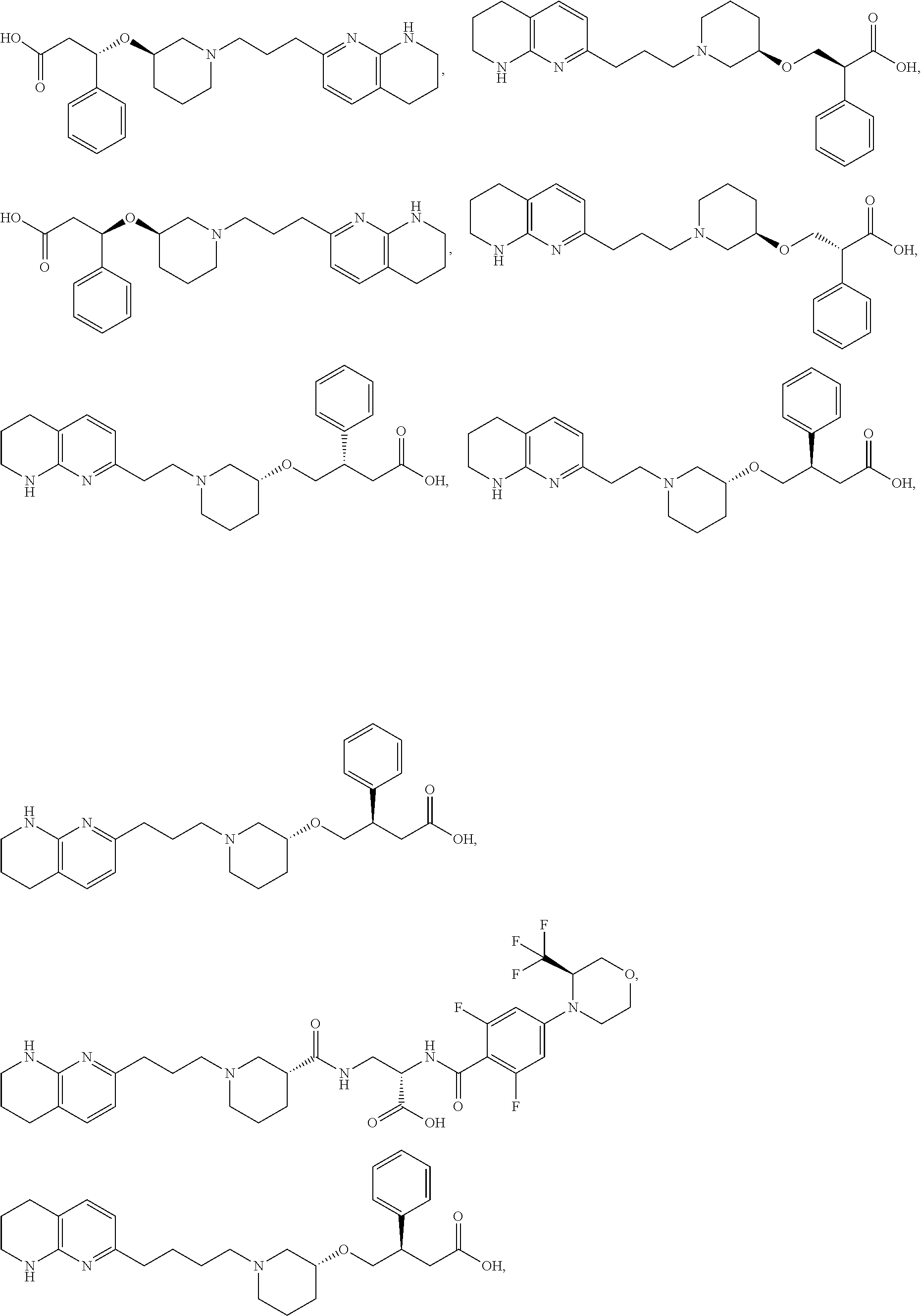 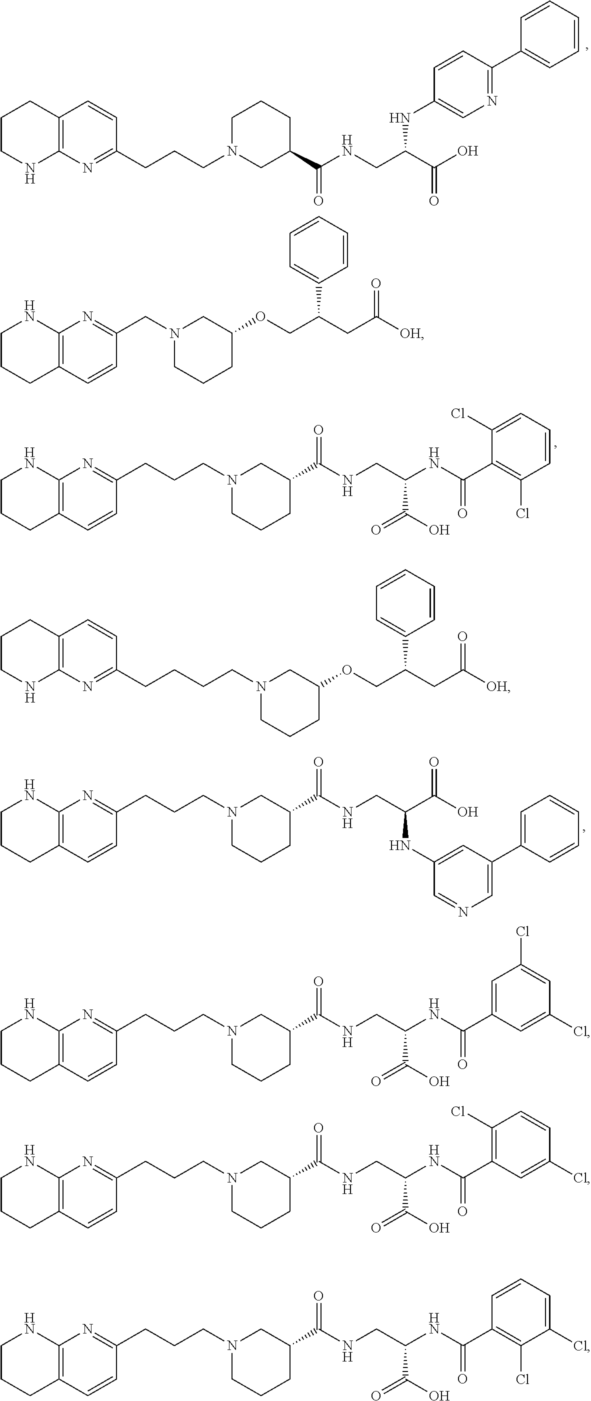 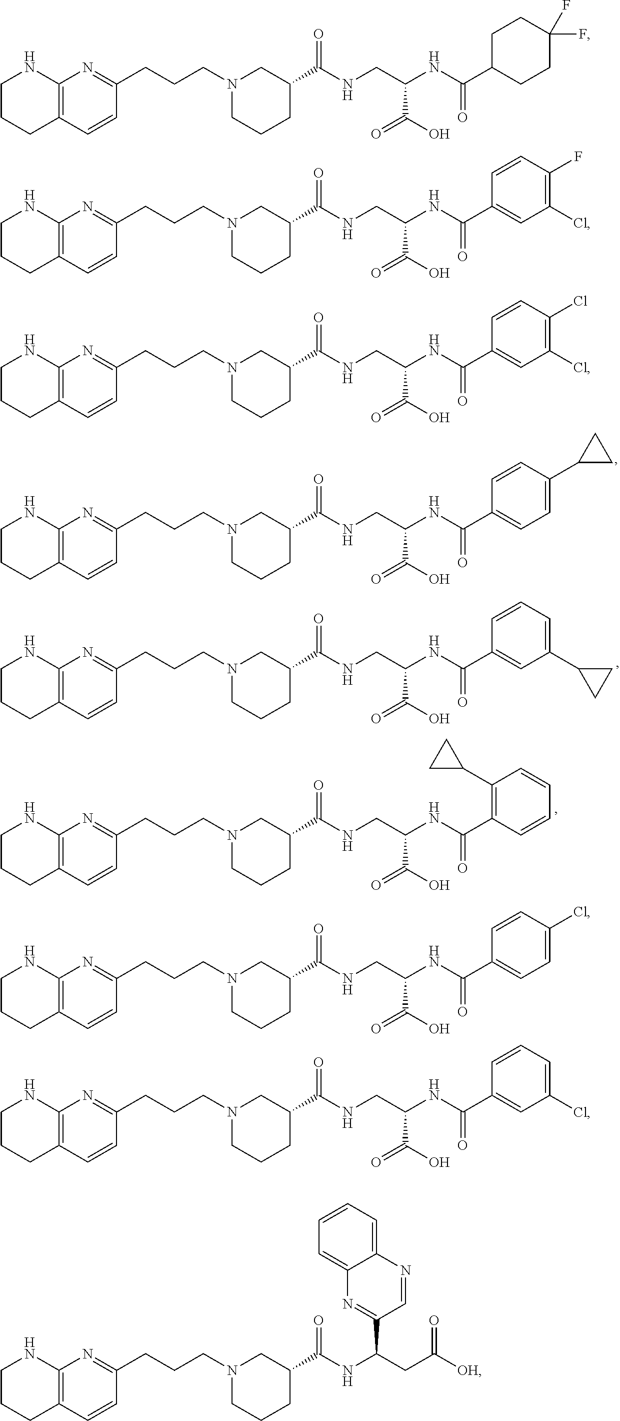 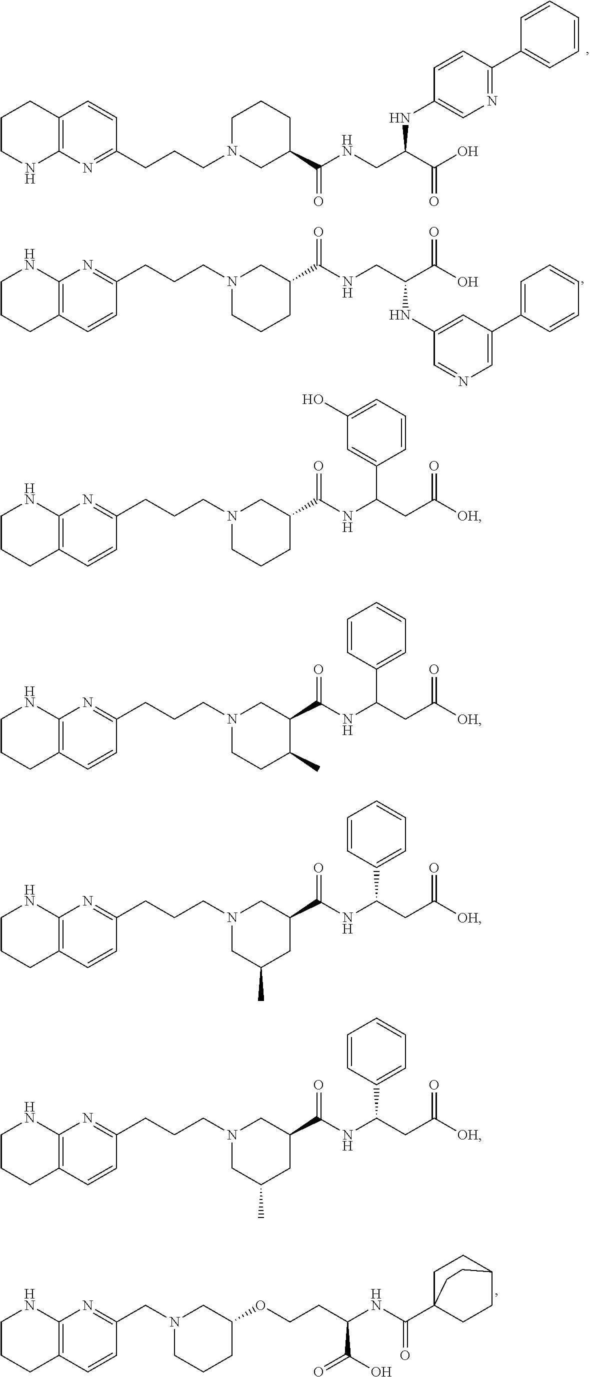 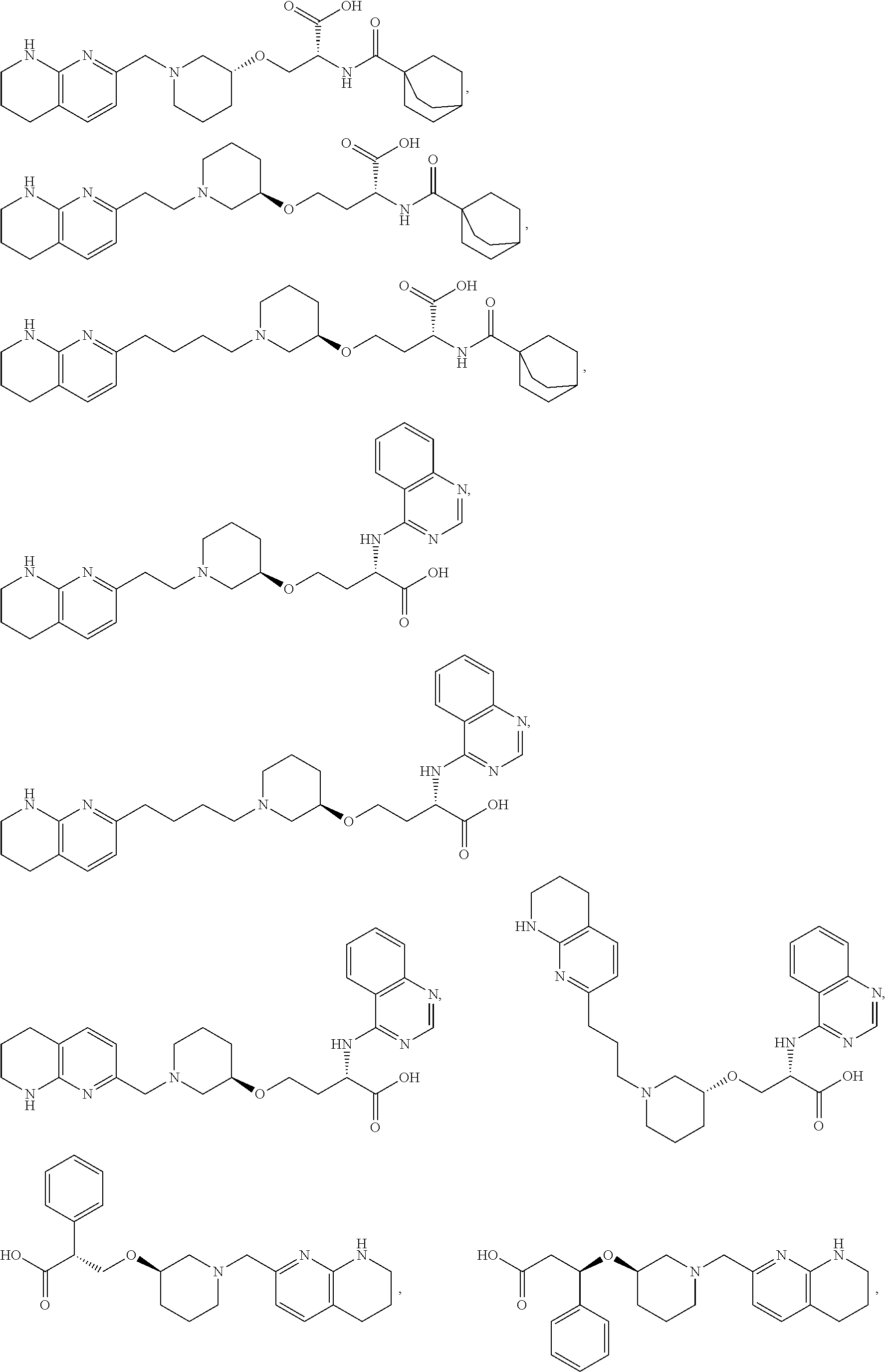 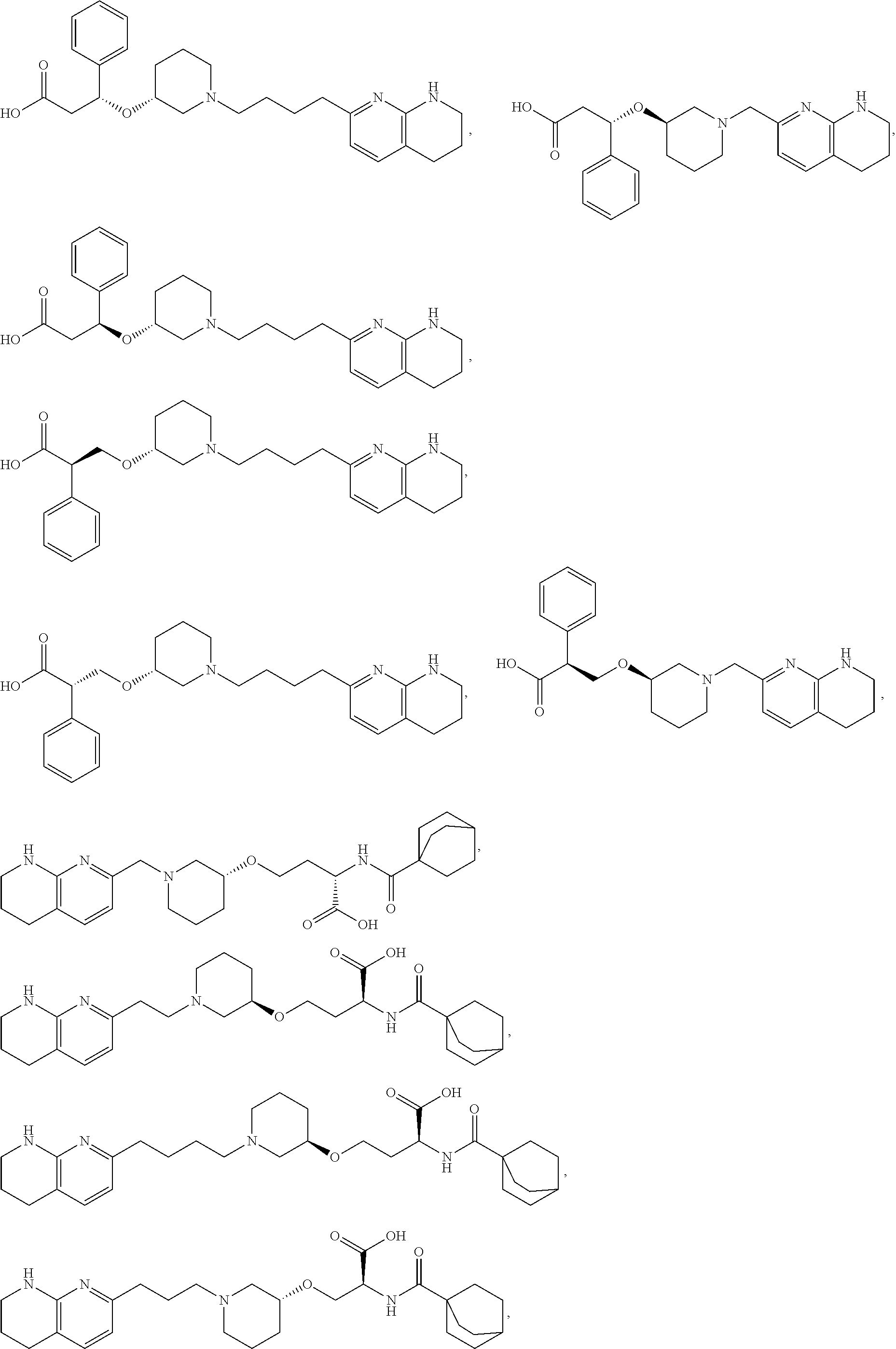 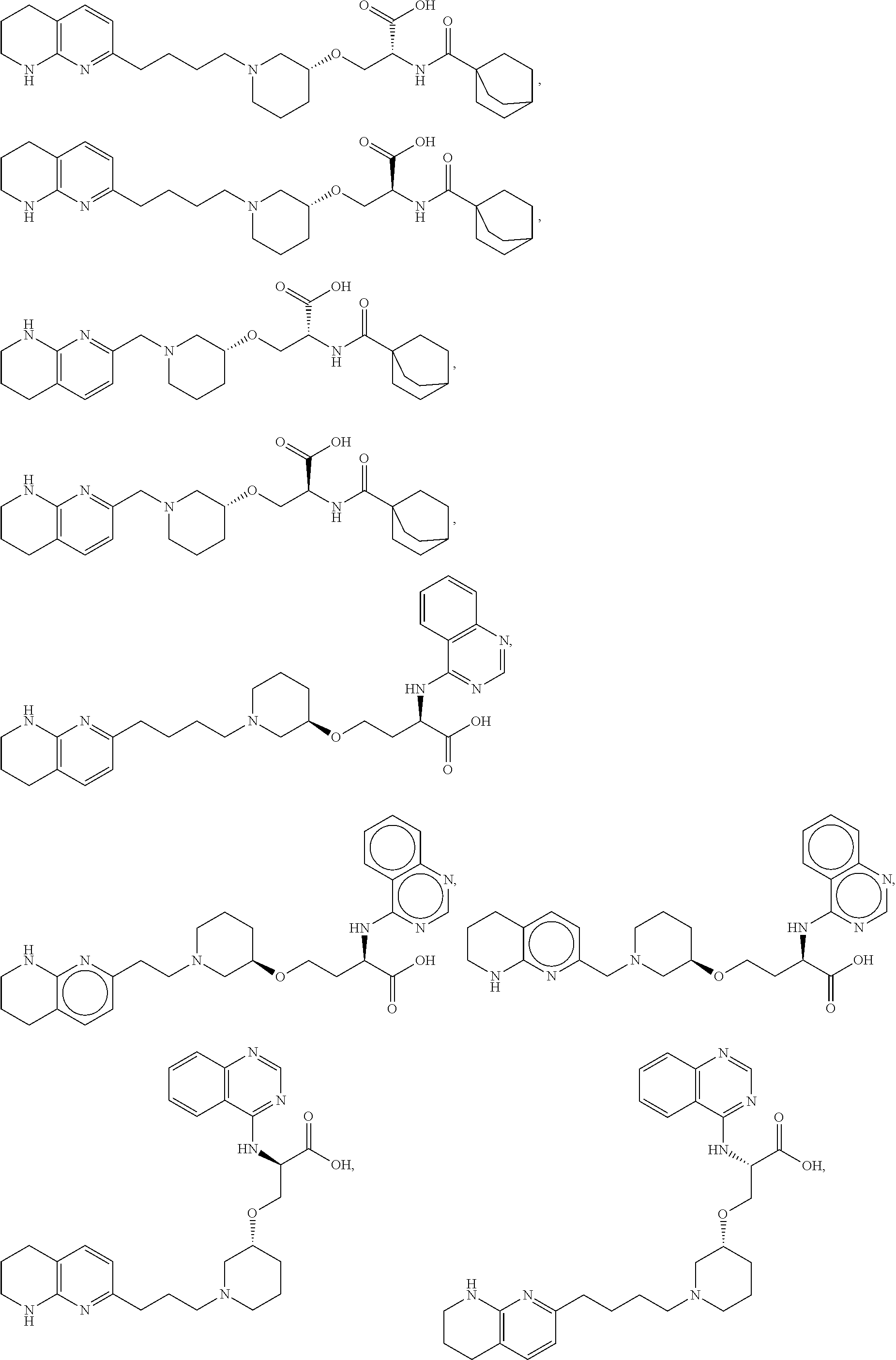 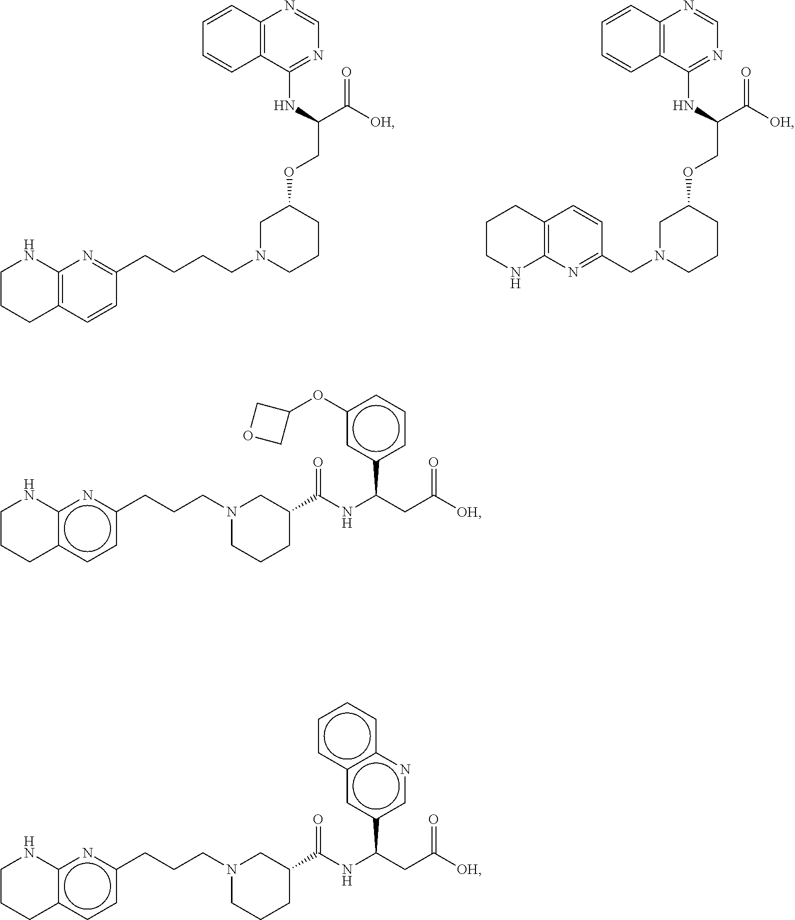 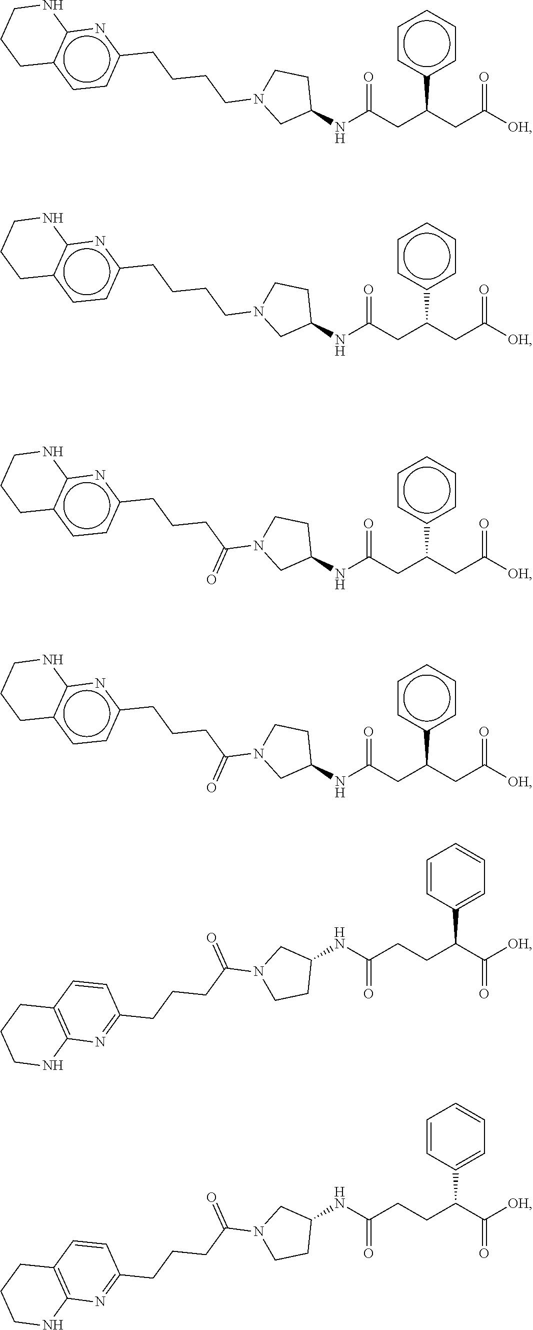 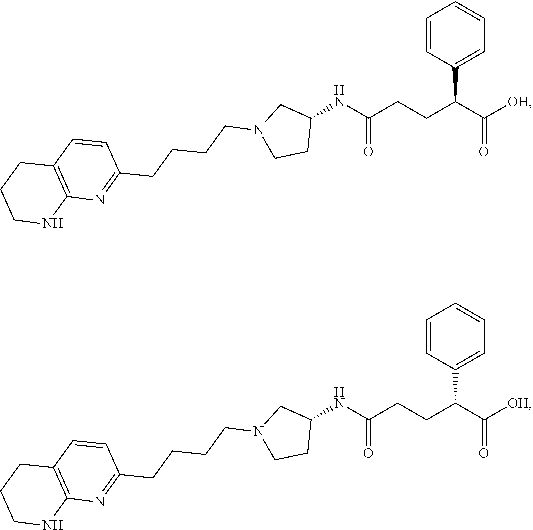 [SMILES: O=C(CC[C@H](C(=O)O)c1ccccc1)N[C@@H]1CCN(CCCCc2ccc3c(n2)NCCC3)C1 ; O=C(CC[C@@H](C(=O)O)c1ccccc1)N[C@@H]1CCN(CCCCc2ccc3c(n2)NCCC3)C1] 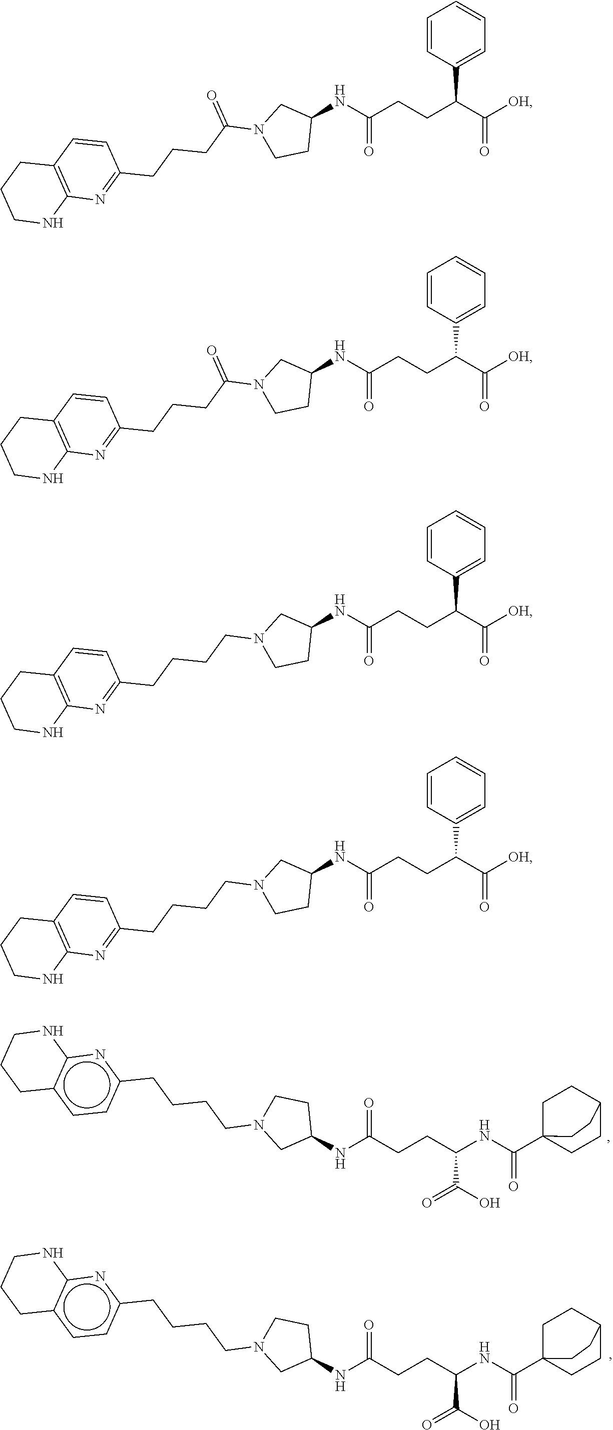 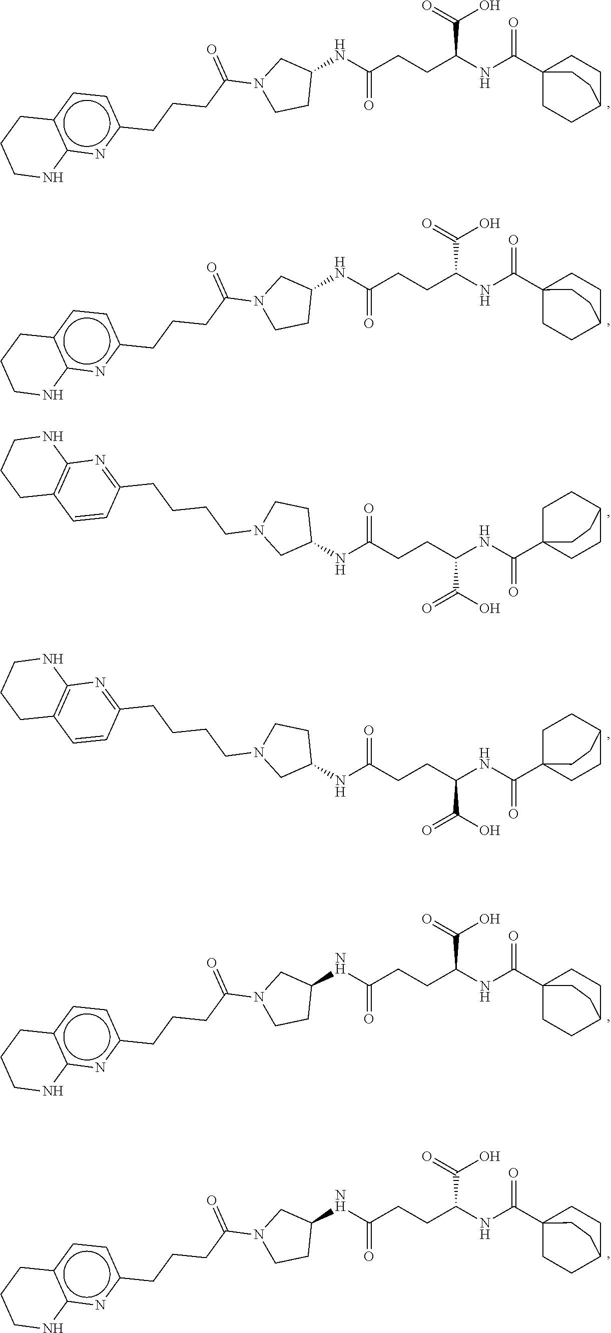 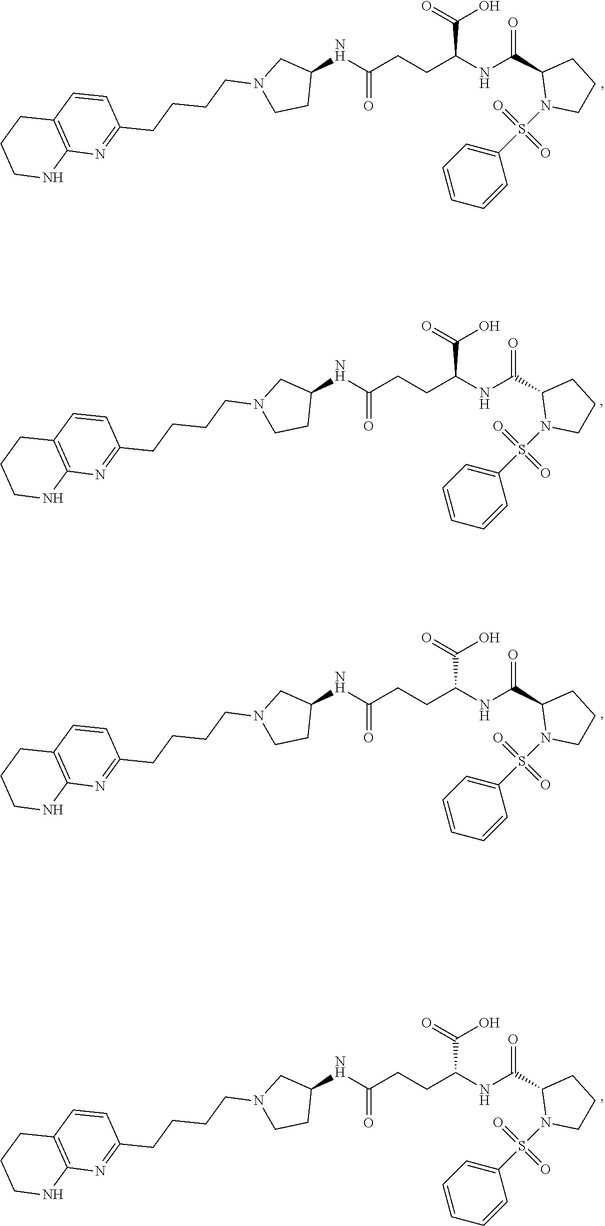 [SMILES: O=C(CC[C@H](NC(=O)[C@H]1CCCN1S(=O)(=O)c1ccccc1)C(=O)O)N[C@H]1CCN(CCCCc2ccc3c(n2)NCCC3)C1 ; O=C(CC[C@H](NC(=O)[C@@H]1CCCN1S(=O)(=O)c1ccccc1)C(=O)O)N[C@H]1CCN(CCCCc2ccc3c(n2)NCCC3)C1 ; O=C(CC[C@@H](NC(=O)[C@H]1CCCN1S(=O)(=O)c1ccccc1)C(=O)O)N[C@H]1CCN(CCCCc2ccc3c(n2)NCCC3)C1 ; O=C(CC[C@@H](NC(=O)[C@@H]1CCCN1S(=O)(=O)c1ccccc1)C(=O)O)N[C@H]1CCN(CCCCc2ccc3c(n2)NCCC3)C1] 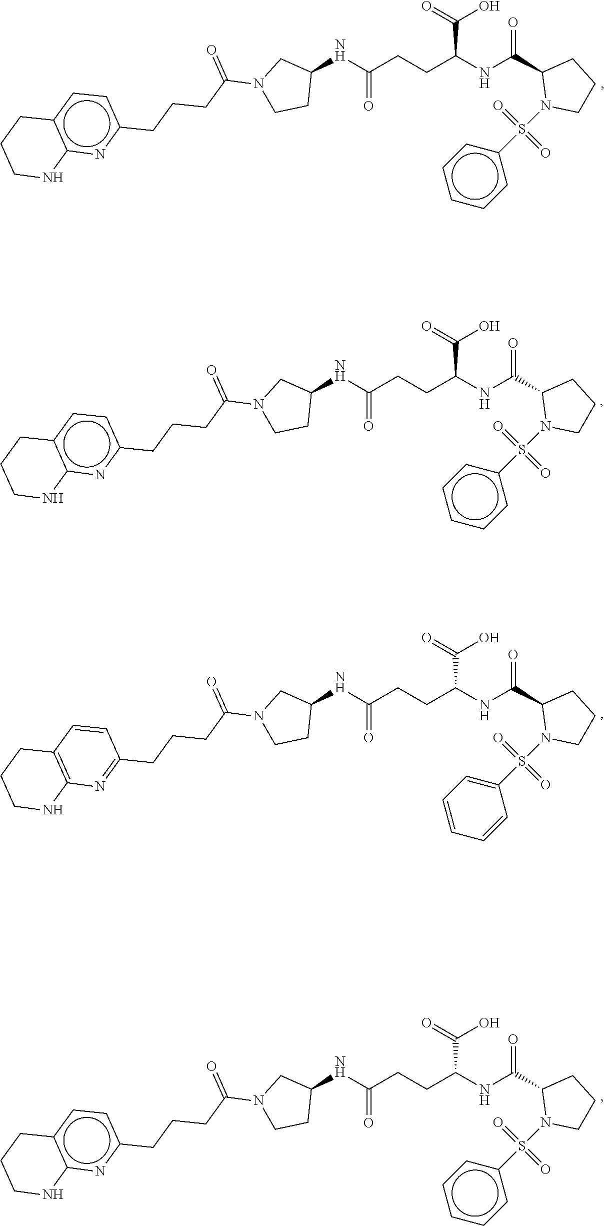 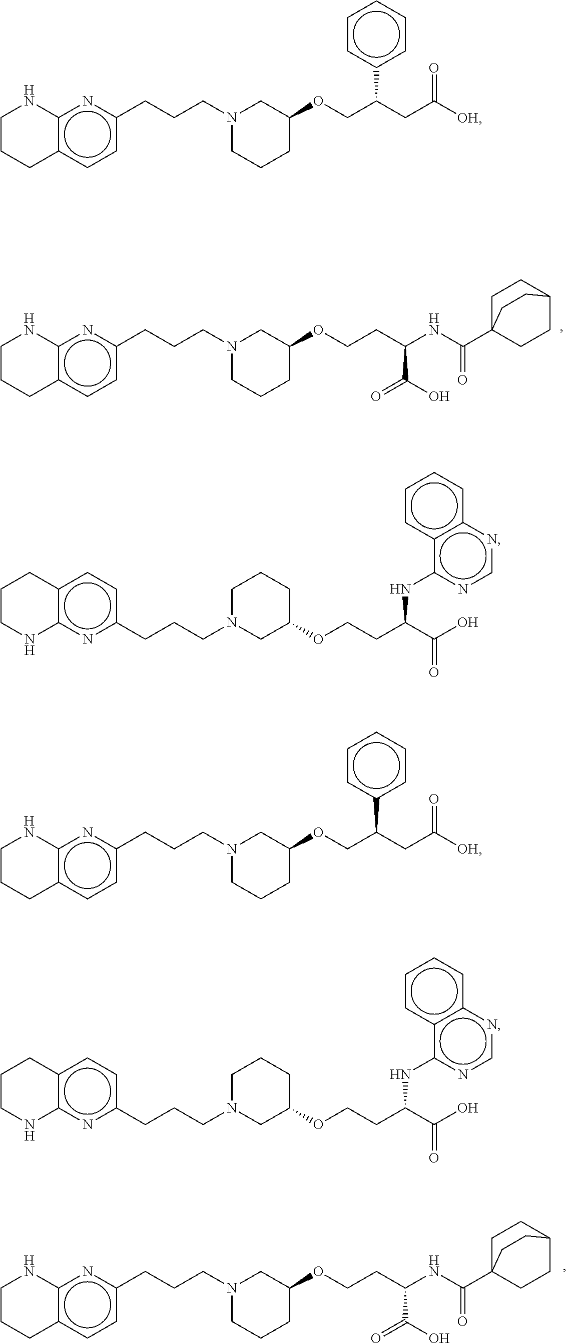 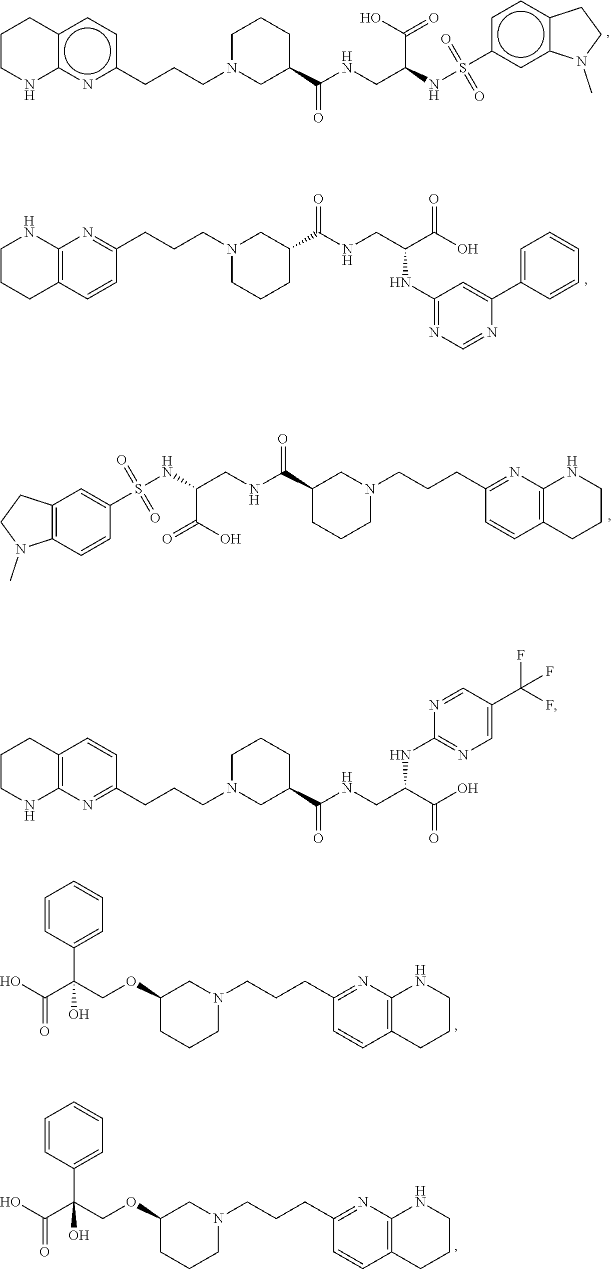 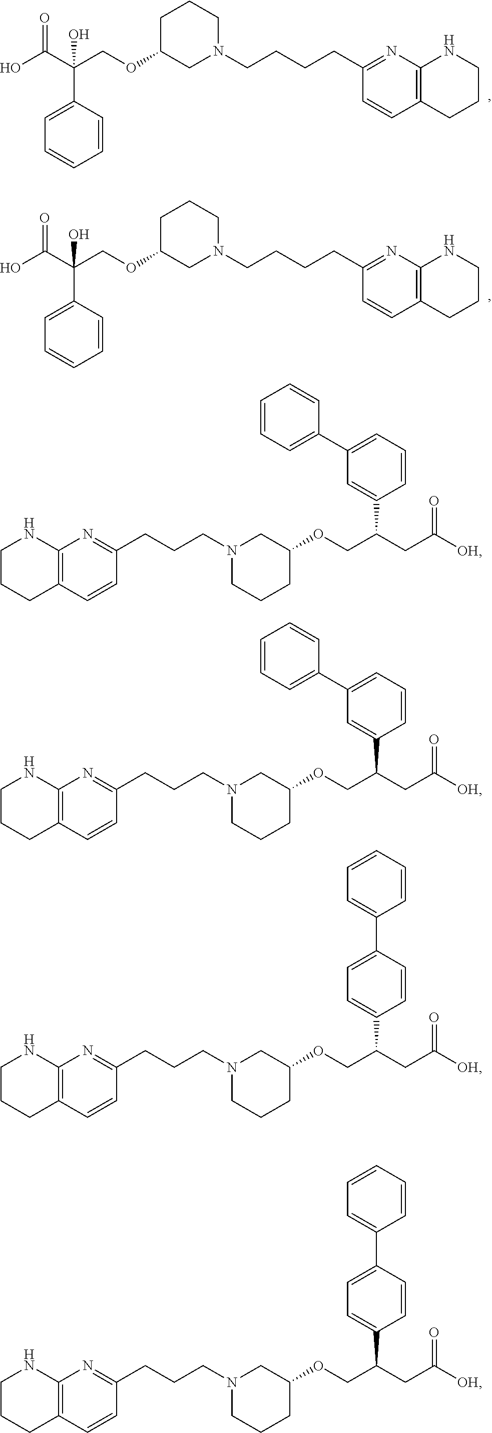 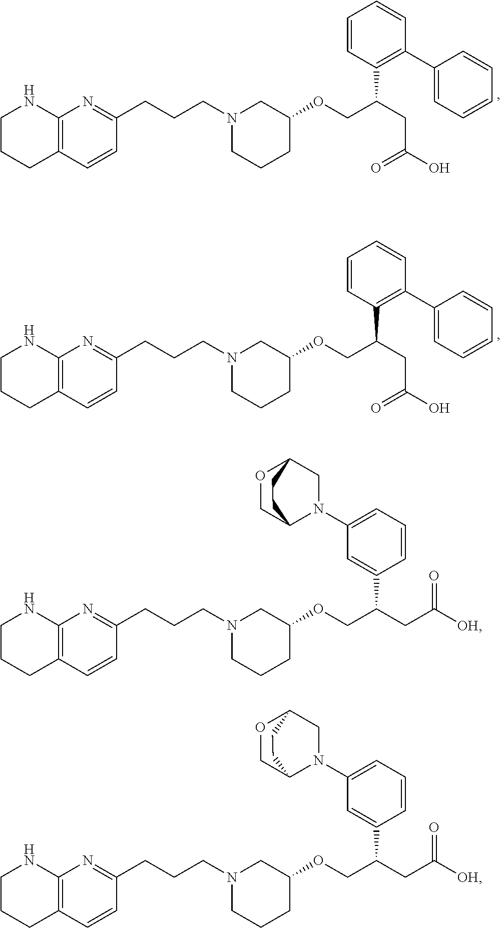 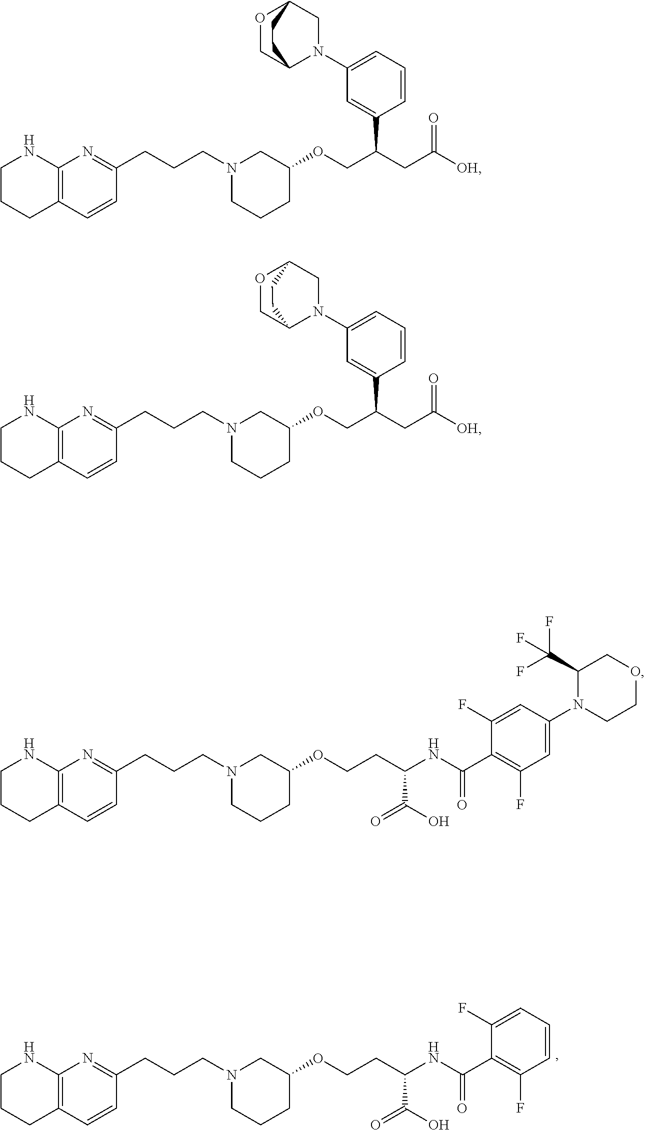 [SMILES: O=C(O)C[C@@H](CO[C@@H]1CCCN(CCCc2ccc3c(n2)NCCC3)C1)c1cccc(N2C[C@@H]3CC[C@H]2CO3)c1 ; O=C(O)C[C@@H](CO[C@@H]1CCCN(CCCc2ccc3c(n2)NCCC3)C1)c1cccc(N2C[C@H]3CC[C@@H]2CO3)c1 ; O=C(N[C@@H](CCO[C@@H]1CCCN(CCCc2ccc3c(n2)NCCC3)C1)C(=O)O)c1c(F)cc(N2CCOC[C@@H]2C(F)(F)F)cc1F ; O=C(N[C@@H](CCO[C@@H]1CCCN(CCCc2ccc3c(n2)NCCC3)C1)C(=O)O)c1c(F)cccc1F] 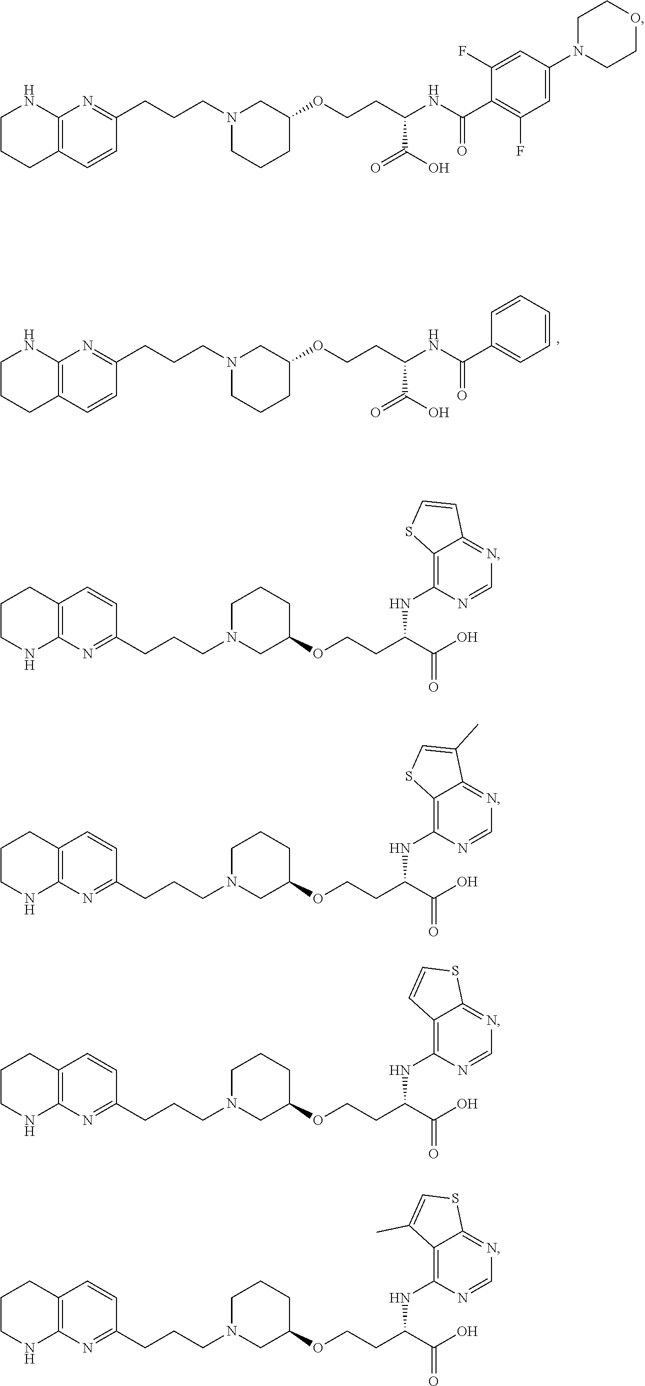 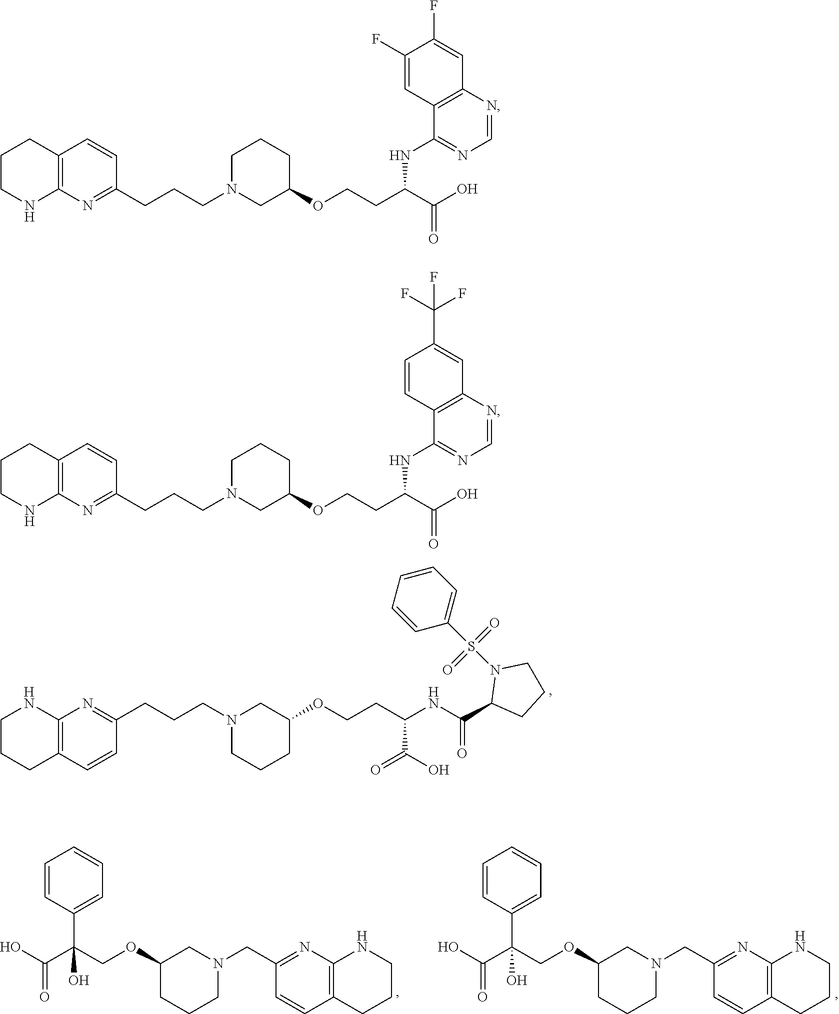 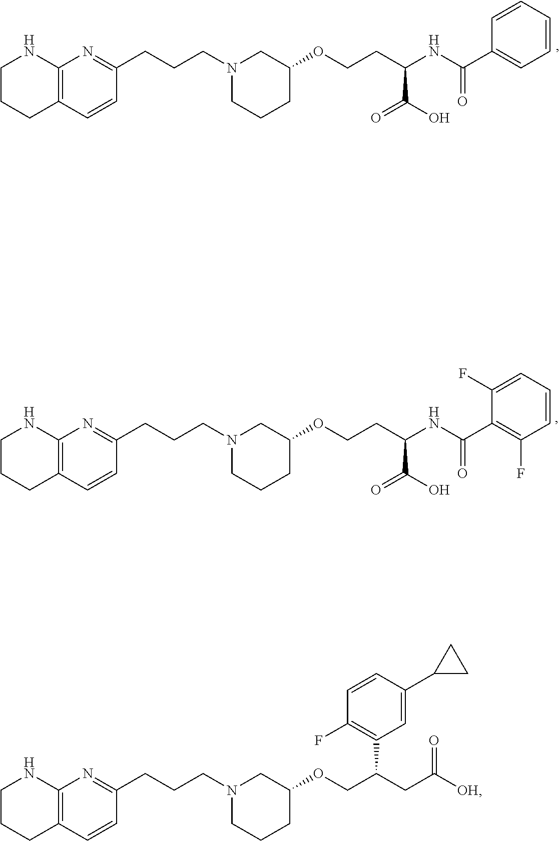 [SMILES: O=C(N[C@H](CCO[C@@H]1CCCN(CCCc2ccc3c(n2)NCCC3)C1)C(=O)O)c1ccccc1 ; O=C(N[C@H](CCO[C@@H]1CCCN(CCCc2ccc3c(n2)NCCC3)C1)C(=O)O)c1c(F)cccc1F ; O=C(O)C[C@H](CO[C@@H]1CCCN(CCCc2ccc3c(n2)NCCC3)C1)c1cc(C2CC2)ccc1F] 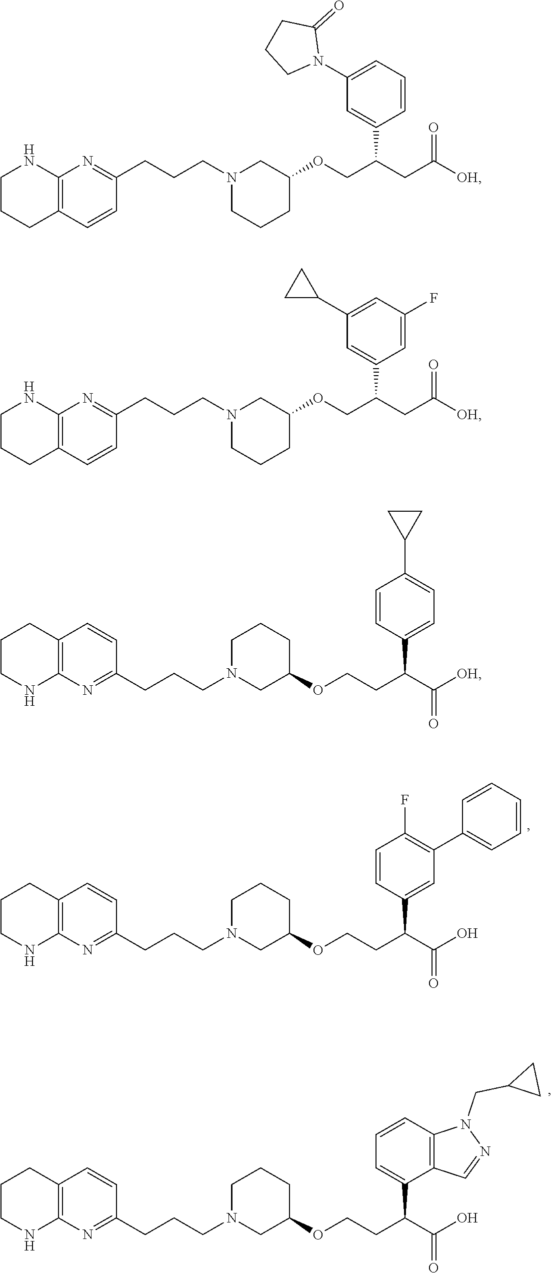 [SMILES: O=C(O)C[C@H](CO[C@@H]1CCCN(CCCc2ccc3c(n2)NCCC3)C1)c1cccc(N2CCCC2=O)c1 ; O=C(O)C[C@H](CO[C@@H]1CCCN(CCCc2ccc3c(n2)NCCC3)C1)c1cc(F)cc(C2CC2)c1 ; O=C(O)[C@@H](CCO[C@@H]1CCCN(CCCc2ccc3c(n2)NCCC3)C1)c1ccc(C2CC2)cc1 ; O=C(O)[C@@H](CCO[C@@H]1CCCN(CCCc2ccc3c(n2)NCCC3)C1)c1ccc(F)c(-c2ccccc2)c1 ; O=C(O)[C@@H](CCO[C@@H]1CCCN(CCCc2ccc3c(n2)NCCC3)C1)c1cccc2c1cnn2CC1CC1] 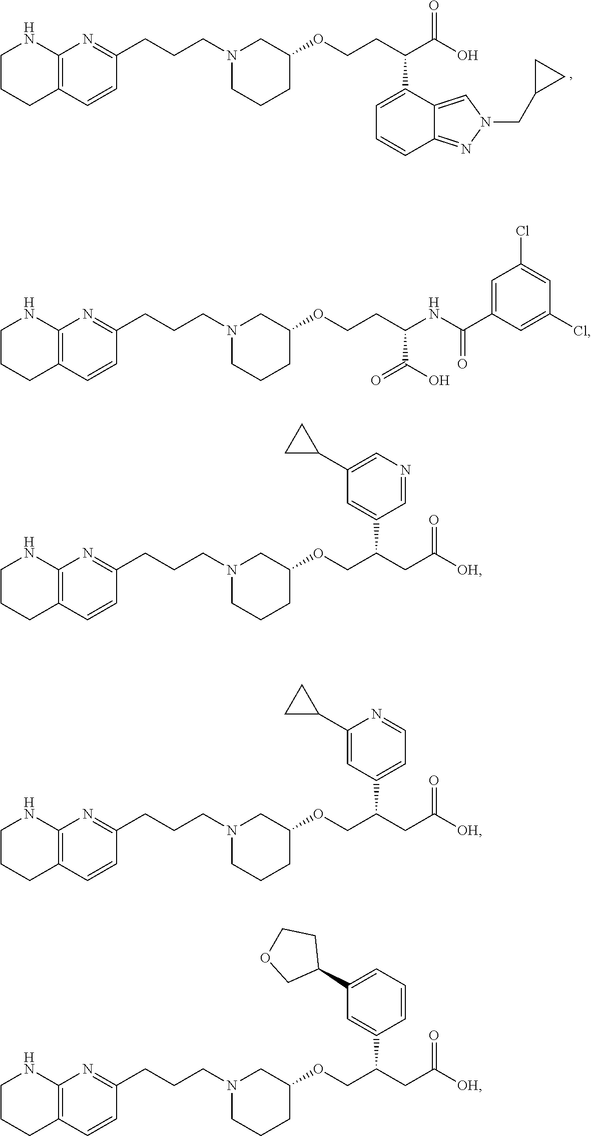 [SMILES: O=C(O)[C@@H](CCO[C@@H]1CCCN(CCCc2ccc3c(n2)NCCC3)C1)c1cccc2nn(CC3CC3)cc12 ; O=C(N[C@@H](CCO[C@@H]1CCCN(CCCc2ccc3c(n2)NCCC3)C1)C(=O)O)c1cc(Cl)cc(Cl)c1 ; O=C(O)C[C@H](CO[C@@H]1CCCN(CCCc2ccc3c(n2)NCCC3)C1)c1cncc(C2CC2)c1 ; O=C(O)C[C@H](CO[C@@H]1CCCN(CCCc2ccc3c(n2)NCCC3)C1)c1ccnc(C2CC2)c1 ; O=C(O)C[C@H](CO[C@@H]1CCCN(CCCc2ccc3c(n2)NCCC3)C1)c1cccc([C@@H]2CCOC2)c1] 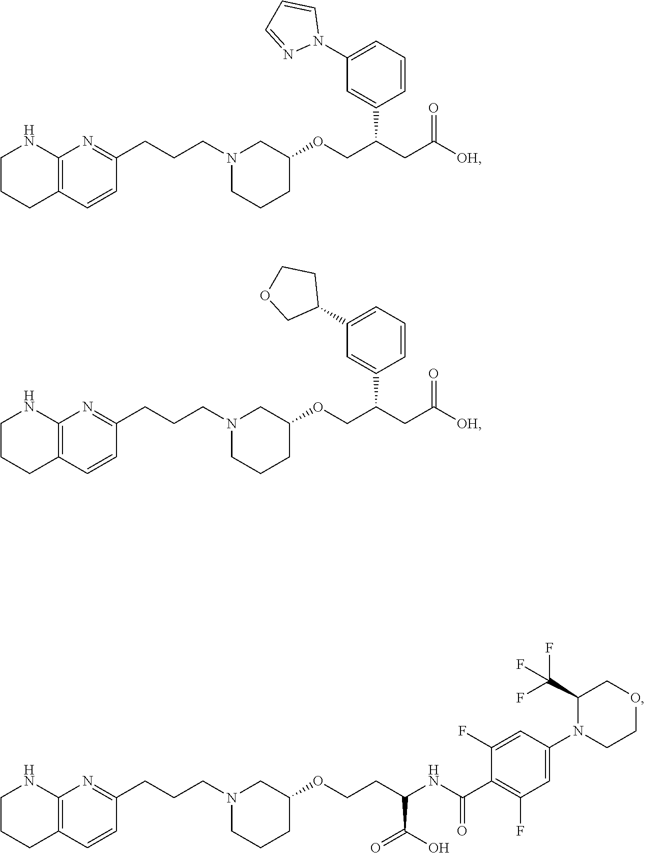 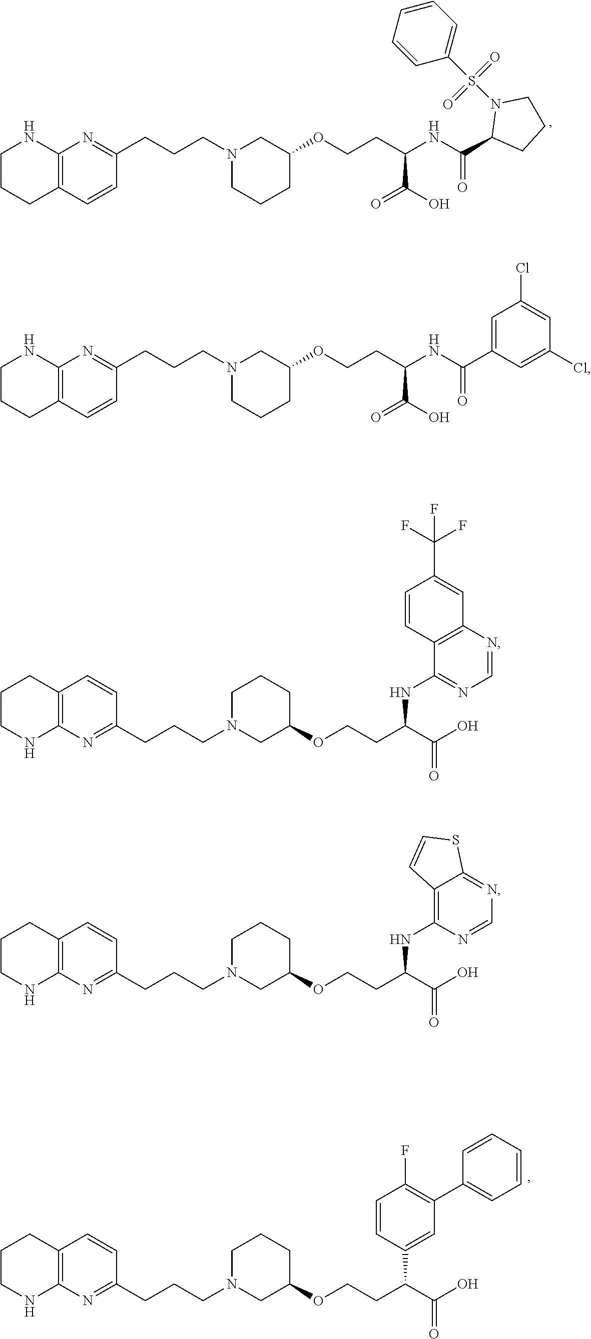 [SMILES: O=C(O)[C@@H](CCO[C@@H]1CCCN(CCCc2ccc3c(n2)NCCC3)C1)NC(=O)[C@@H]1CCCN1S(=O)(=O)c1ccccc1 ; O=C(N[C@H](CCO[C@@H]1CCCN(CCCc2ccc3c(n2)NCCC3)C1)C(=O)O)c1cc(Cl)cc(Cl)c1 ; O=C(O)[C@@H](CCO[C@@H]1CCCN(CCCc2ccc3c(n2)NCCC3)C1)Nc1ncnc2cc(C(F)(F)F)ccc12 ; O=C(O)[C@@H](CCO[C@@H]1CCCN(CCCc2ccc3c(n2)NCCC3)C1)Nc1ncnc2sccc12 ; O=C(O)[C@H](CCO[C@@H]1CCCN(CCCc2ccc3c(n2)NCCC3)C1)c1ccc(F)c(-c2ccccc2)c1] 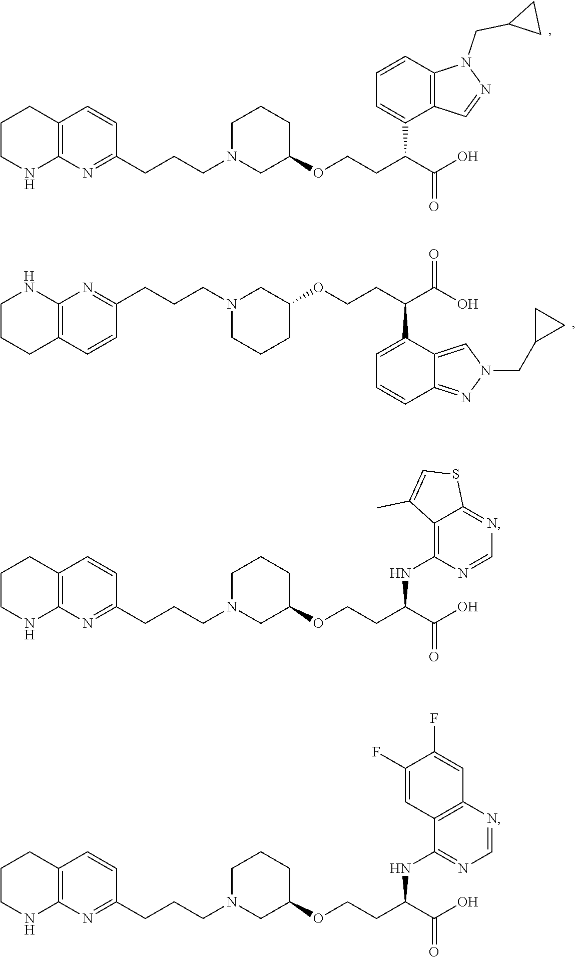 [SMILES: O=C(O)[C@H](CCO[C@@H]1CCCN(CCCc2ccc3c(n2)NCCC3)C1)c1cccc2c1cnn2CC1CC1 ; O=C(O)[C@H](CCO[C@@H]1CCCN(CCCc2ccc3c(n2)NCCC3)C1)c1cccc2nn(CC3CC3)cc12 ; Cc1csc2ncnc(N[C@H](CCO[C@@H]3CCCN(CCCc4ccc5c(n4)NCCC5)C3)C(=O)O)c12 ; O=C(O)[C@@H](CCO[C@@H]1CCCN(CCCc2ccc3c(n2)NCCC3)C1)Nc1ncnc2cc(F)c(F)cc12] 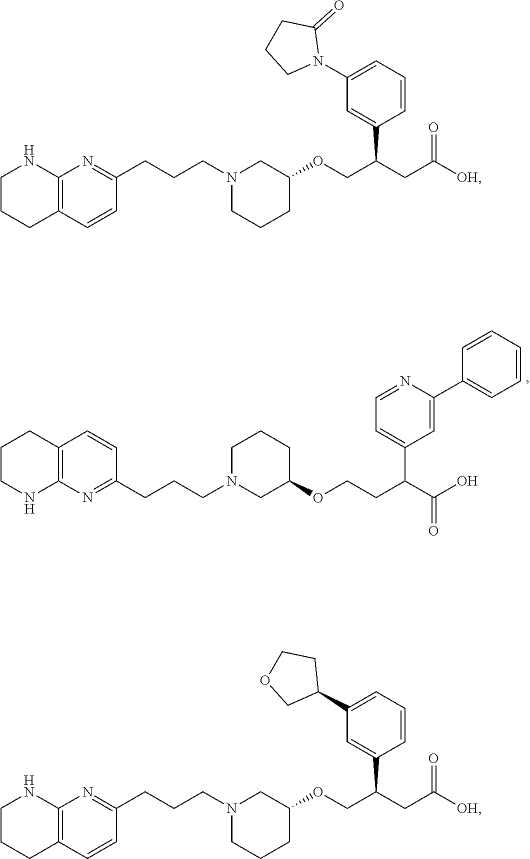 [SMILES: O=C(O)C[C@@H](CO[C@@H]1CCCN(CCCc2ccc3c(n2)NCCC3)C1)c1cccc(N2CCCC2=O)c1 ; O=C(O)C(CCO[C@@H]1CCCN(CCCc2ccc3c(n2)NCCC3)C1)c1ccnc(-c2ccccc2)c1 ; O=C(O)C[C@@H](CO[C@@H]1CCCN(CCCc2ccc3c(n2)NCCC3)C1)c1cccc([C@@H]2CCOC2)c1] 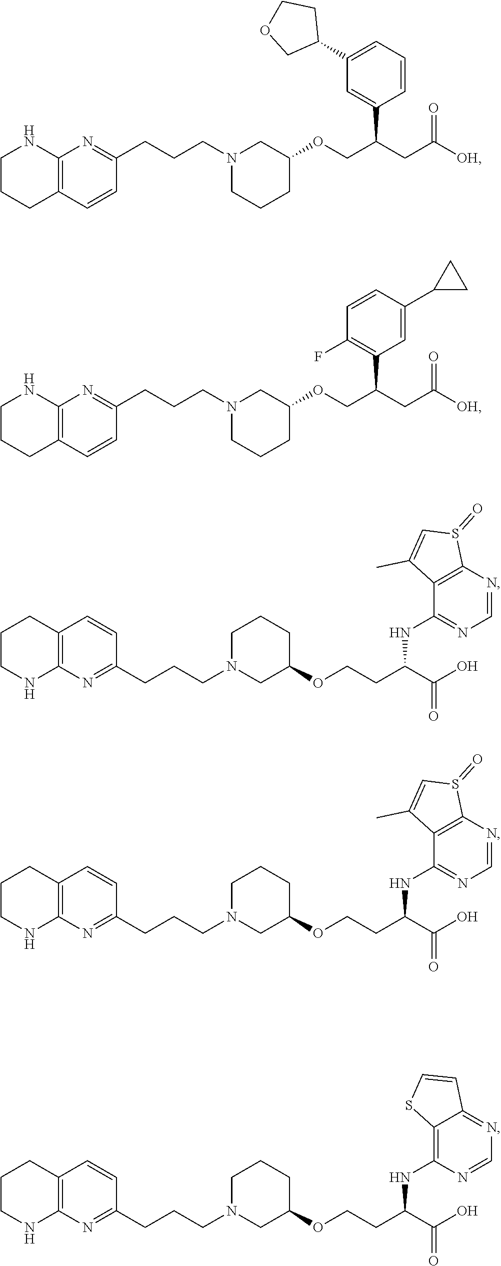 [SMILES: O=C(O)C[C@@H](CO[C@@H]1CCCN(CCCc2ccc3c(n2)NCCC3)C1)c1cccc([C@H]2CCOC2)c1 ; O=C(O)C[C@@H](CO[C@@H]1CCCN(CCCc2ccc3c(n2)NCCC3)C1)c1cc(C2CC2)ccc1F ; CC1=CS(=O)c2ncnc(N[C@@H](CCO[C@@H]3CCCN(CCCc4ccc5c(n4)NCCC5)C3)C(=O)O)c21 ; CC1=CS(=O)c2ncnc(N[C@H](CCO[C@@H]3CCCN(CCCc4ccc5c(n4)NCCC5)C3)C(=O)O)c21 ; O=C(O)[C@@H](CCO[C@@H]1CCCN(CCCc2ccc3c(n2)NCCC3)C1)Nc1ncnc2ccsc12] 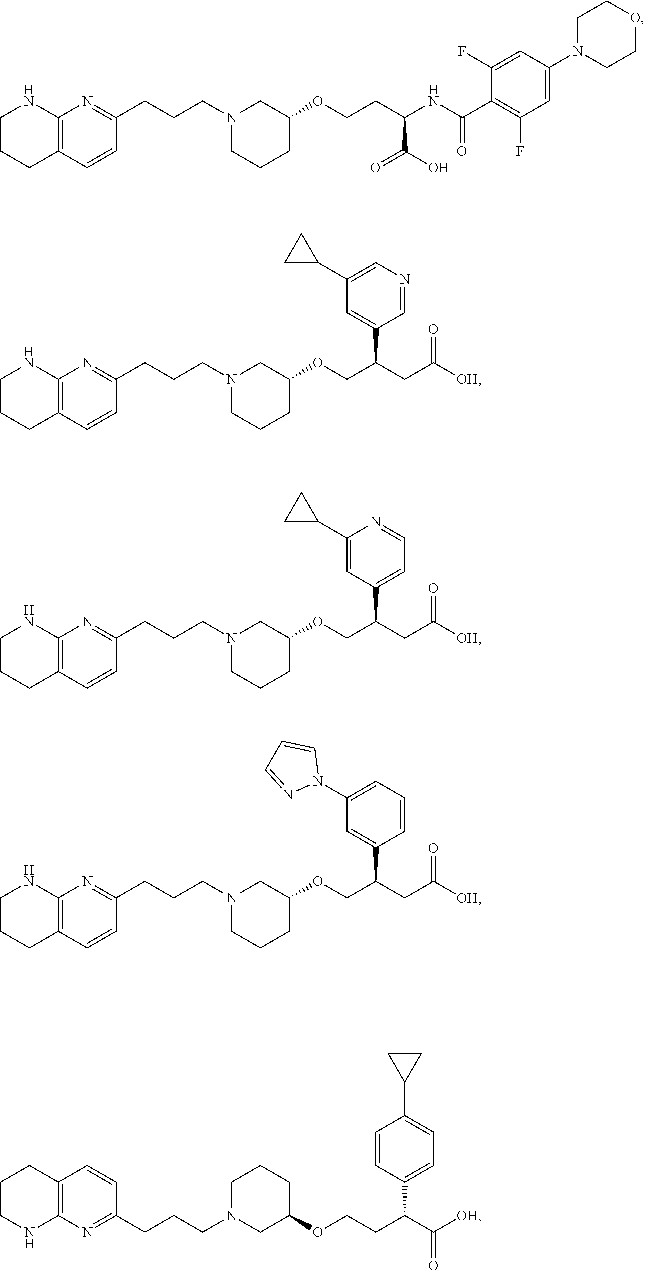 [SMILES: O=C(N[C@H](CCO[C@@H]1CCCN(CCCc2ccc3c(n2)NCCC3)C1)C(=O)O)c1c(F)cc(N2CCOCC2)cc1F ; O=C(O)C[C@@H](CO[C@@H]1CCCN(CCCc2ccc3c(n2)NCCC3)C1)c1cncc(C2CC2)c1 ; O=C(O)C[C@@H](CO[C@@H]1CCCN(CCCc2ccc3c(n2)NCCC3)C1)c1ccnc(C2CC2)c1 ; O=C(O)C[C@@H](CO[C@@H]1CCCN(CCCc2ccc3c(n2)NCCC3)C1)c1cccc(-n2cccn2)c1 ; O=C(O)[C@H](CCO[C@@H]1CCCN(CCCc2ccc3c(n2)NCCC3)C1)c1ccc(C2CC2)cc1] 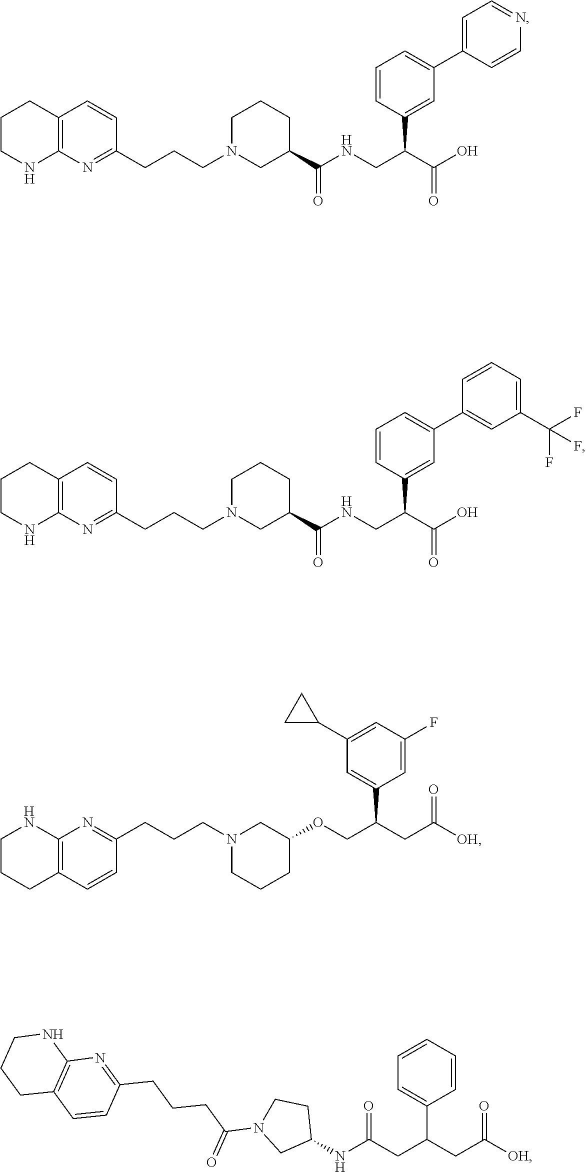 [SMILES: O=C(NC[C@H](C(=O)O)c1cccc(-c2ccncc2)c1)[C@@H]1CCCN(CCCc2ccc3c(n2)NCCC3)C1 ; O=C(NC[C@H](C(=O)O)c1cccc(-c2cccc(C(F)(F)F)c2)c1)[C@@H]1CCCN(CCCc2ccc3c(n2)NCCC3)C1 ; O=C(O)C[C@@H](CO[C@@H]1CCCN(CCCc2ccc3c(n2)NCCC3)C1)c1cc(F)cc(C2CC2)c1 ; O=C(O)CC(CC(=O)N[C@H]1CCN(C(=O)CCCc2ccc3c(n2)NCCC3)C1)c1ccccc1] 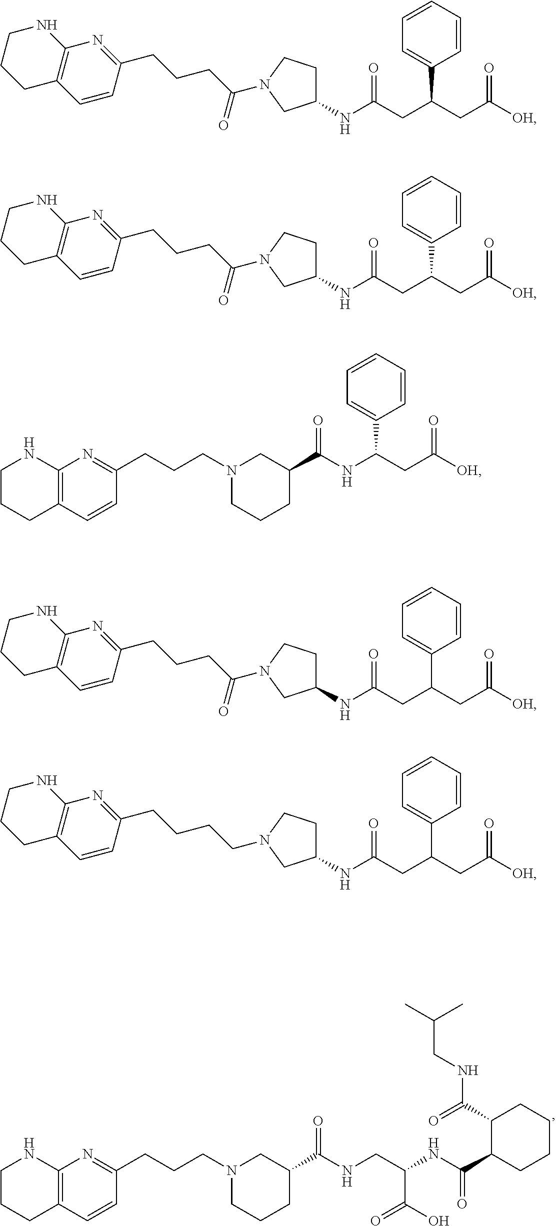 [SMILES: O=C(O)C[C@@H](CC(=O)N[C@H]1CCN(C(=O)CCCc2ccc3c(n2)NCCC3)C1)c1ccccc1 ; O=C(O)C[C@H](CC(=O)N[C@H]1CCN(C(=O)CCCc2ccc3c(n2)NCCC3)C1)c1ccccc1 ; O=C(O)C[C@H](NC(=O)[C@H]1CCCN(CCCc2ccc3c(n2)NCCC3)C1)c1ccccc1 ; O=C(O)CC(CC(=O)N[C@@H]1CCN(C(=O)CCCc2ccc3c(n2)NCCC3)C1)c1ccccc1 ; O=C(O)CC(CC(=O)N[C@H]1CCN(CCCCc2ccc3c(n2)NCCC3)C1)c1ccccc1 ; CC(C)CNC(=O)[C@@H]1CCCC[C@H]1C(=O)N[C@@H](CNC(=O)[C@@H]1CCCN(CCCc2ccc3c(n2)NCCC3)C1)C(=O)O] 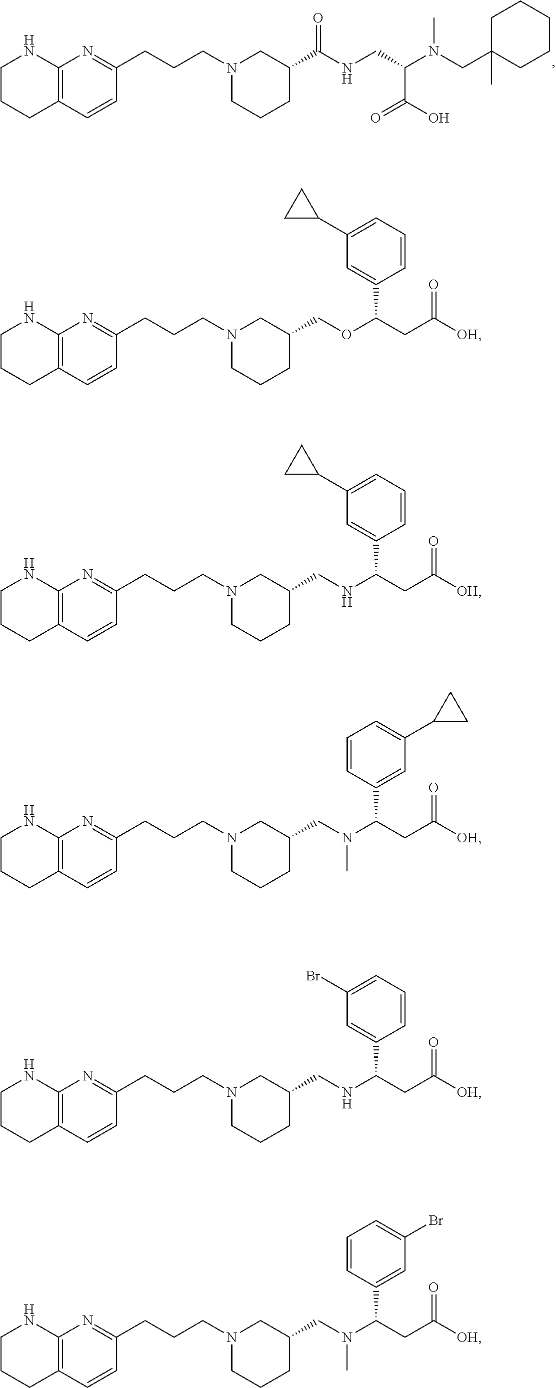 [SMILES: CN(CC1(C)CCCCC1)[C@@H](CNC(=O)[C@@H]1CCCN(CCCc2ccc3c(n2)NCCC3)C1)C(=O)O ; O=C(O)C[C@H](OC[C@@H]1CCCN(CCCc2ccc3c(n2)NCCC3)C1)c1cccc(C2CC2)c1 ; O=C(O)C[C@H](NC[C@@H]1CCCN(CCCc2ccc3c(n2)NCCC3)C1)c1cccc(C2CC2)c1 ; CN(C[C@@H]1CCCN(CCCc2ccc3c(n2)NCCC3)C1)[C@@H](CC(=O)O)c1cccc(C2CC2)c1 ; O=C(O)C[C@H](NC[C@@H]1CCCN(CCCc2ccc3c(n2)NCCC3)C1)c1cccc(Br)c1 ; CN(C[C@@H]1CCCN(CCCc2ccc3c(n2)NCCC3)C1)[C@@H](CC(=O)O)c1cccc(Br)c1] 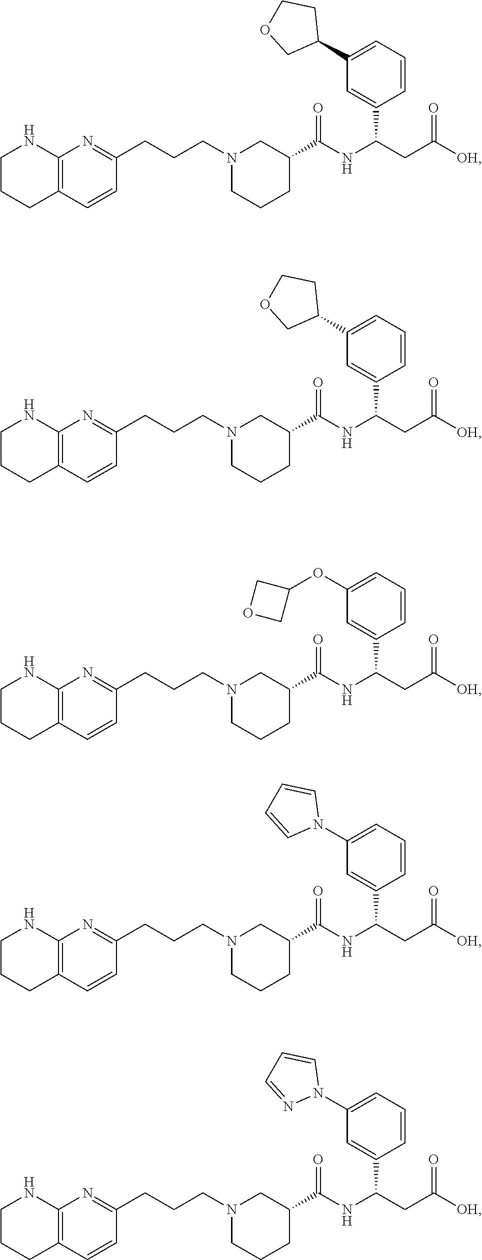 [SMILES: O=C(O)C[C@H](NC(=O)[C@@H]1CCCN(CCCc2ccc3c(n2)NCCC3)C1)c1cccc([C@@H]2CCOC2)c1 ; O=C(O)C[C@H](NC(=O)[C@@H]1CCCN(CCCc2ccc3c(n2)NCCC3)C1)c1cccc([C@H]2CCOC2)c1 ; O=C(O)C[C@H](NC(=O)[C@@H]1CCCN(CCCc2ccc3c(n2)NCCC3)C1)c1cccc(OC2COC2)c1 ; O=C(O)C[C@H](NC(=O)[C@@H]1CCCN(CCCc2ccc3c(n2)NCCC3)C1)c1cccc(-n2cccc2)c1 ; O=C(O)C[C@H](NC(=O)[C@@H]1CCCN(CCCc2ccc3c(n2)NCCC3)C1)c1cccc(-n2cccn2)c1] 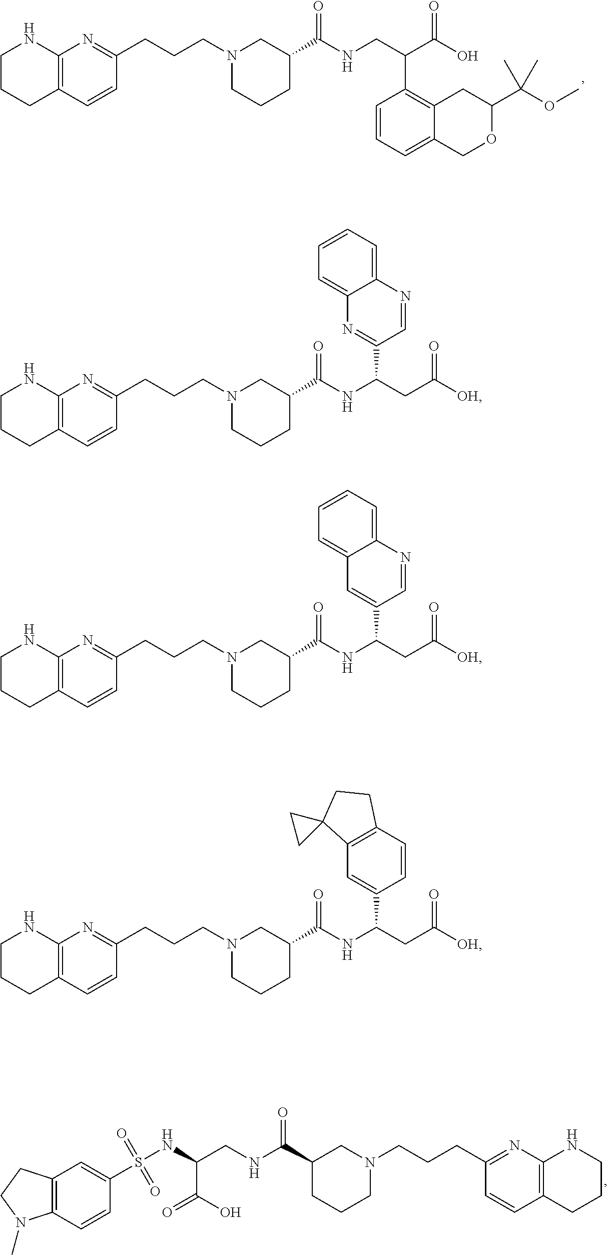 [SMILES: COC(C)(C)C1Cc2c(cccc2C(CNC(=O)[C@@H]2CCCN(CCCc3ccc4c(n3)NCCC4)C2)C(=O)O)CO1 ; O=C(O)C[C@H](NC(=O)[C@@H]1CCCN(CCCc2ccc3c(n2)NCCC3)C1)c1cnc2ccccc2n1 ; O=C(O)C[C@H](NC(=O)[C@@H]1CCCN(CCCc2ccc3c(n2)NCCC3)C1)c1cnc2ccccc2c1 ; O=C(O)C[C@H](NC(=O)[C@@H]1CCCN(CCCc2ccc3c(n2)NCCC3)C1)c1ccc2c(c1)C1(CC2)CC1 ; CN1CCc2cc(S(=O)(=O)N[C@@H](CNC(=O)[C@@H]3CCCN(CCCc4ccc5c(n4)NCCC5)C3)C(=O)O)ccc21] 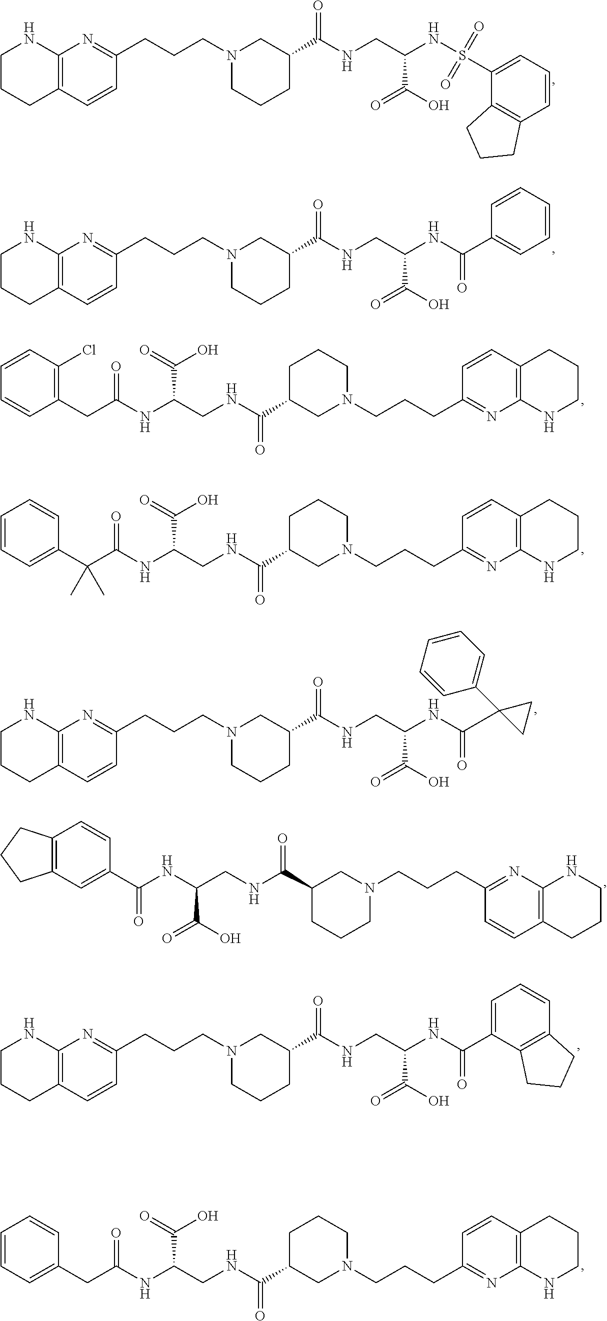 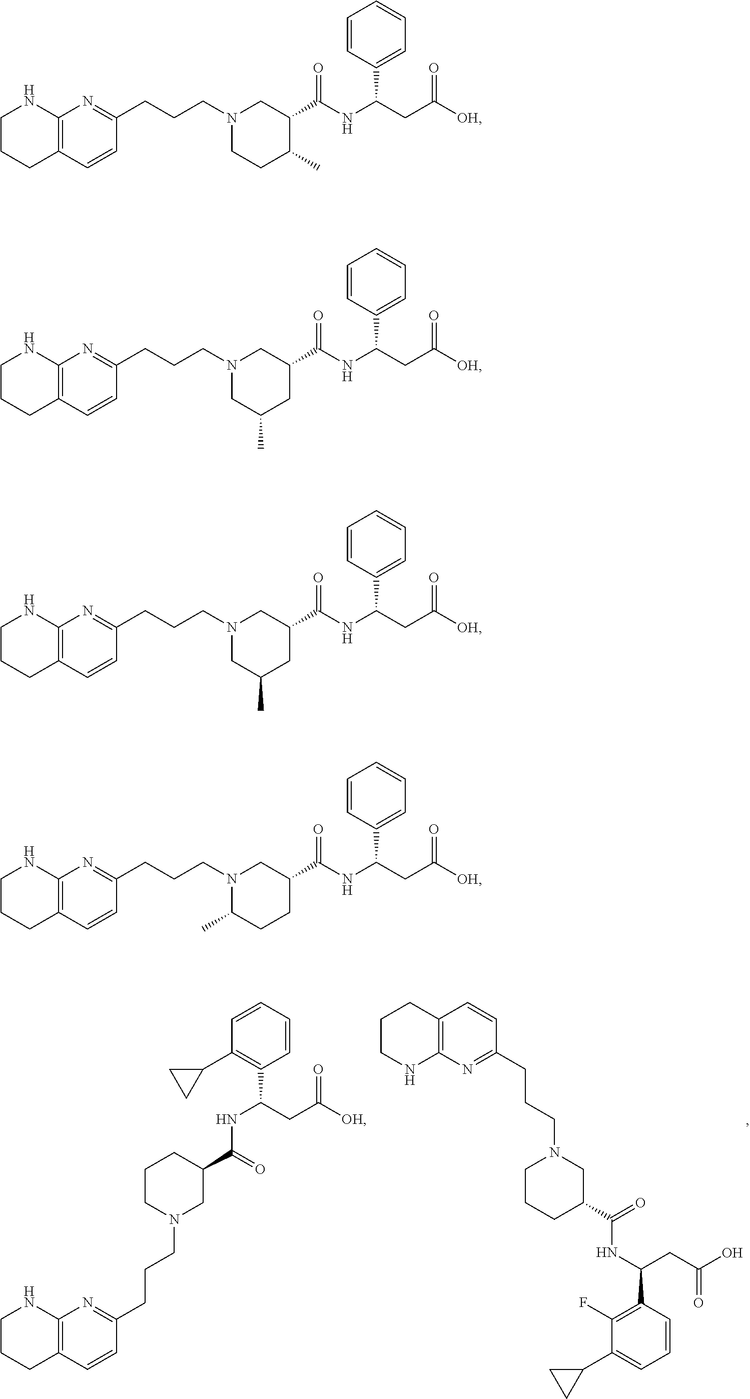 [SMILES: C[C@@H]1CCN(CCCc2ccc3c(n2)NCCC3)C[C@@H]1C(=O)N[C@@H](CC(=O)O)c1ccccc1 ; C[C@H]1C[C@@H](C(=O)N[C@@H](CC(=O)O)c2ccccc2)CN(CCCc2ccc3c(n2)NCCC3)C1 ; C[C@@H]1C[C@@H](C(=O)N[C@@H](CC(=O)O)c2ccccc2)CN(CCCc2ccc3c(n2)NCCC3)C1 ; C[C@H]1CC[C@@H](C(=O)N[C@@H](CC(=O)O)c2ccccc2)CN1CCCc1ccc2c(n1)NCCC2 ; O=C(O)C[C@H](NC(=O)[C@@H]1CCCN(CCCc2ccc3c(n2)NCCC3)C1)c1ccccc1C1CC1 ; O=C(O)C[C@H](NC(=O)[C@@H]1CCCN(CCCc2ccc3c(n2)NCCC3)C1)c1cccc(C2CC2)c1F] 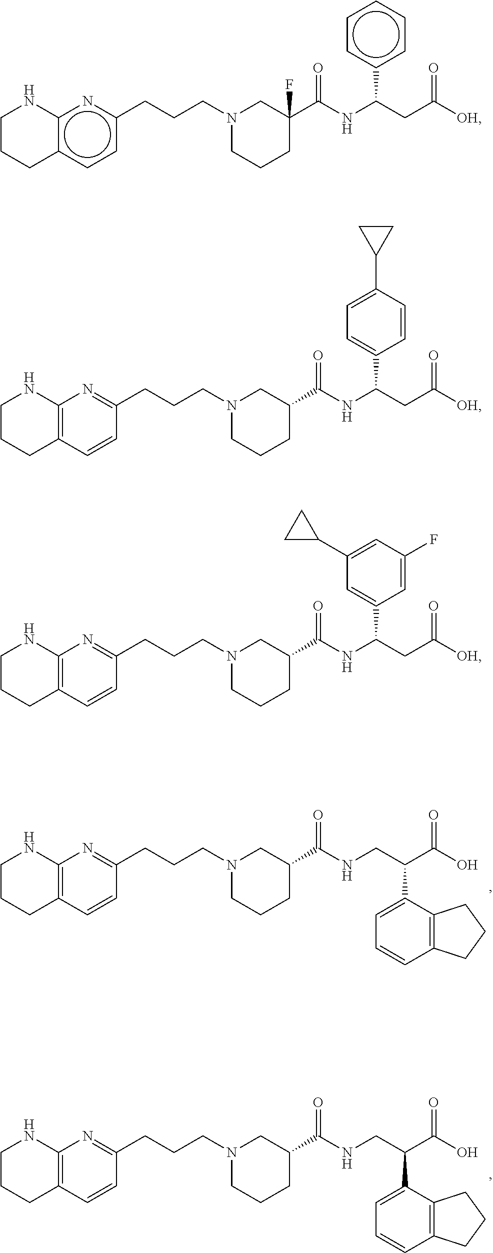 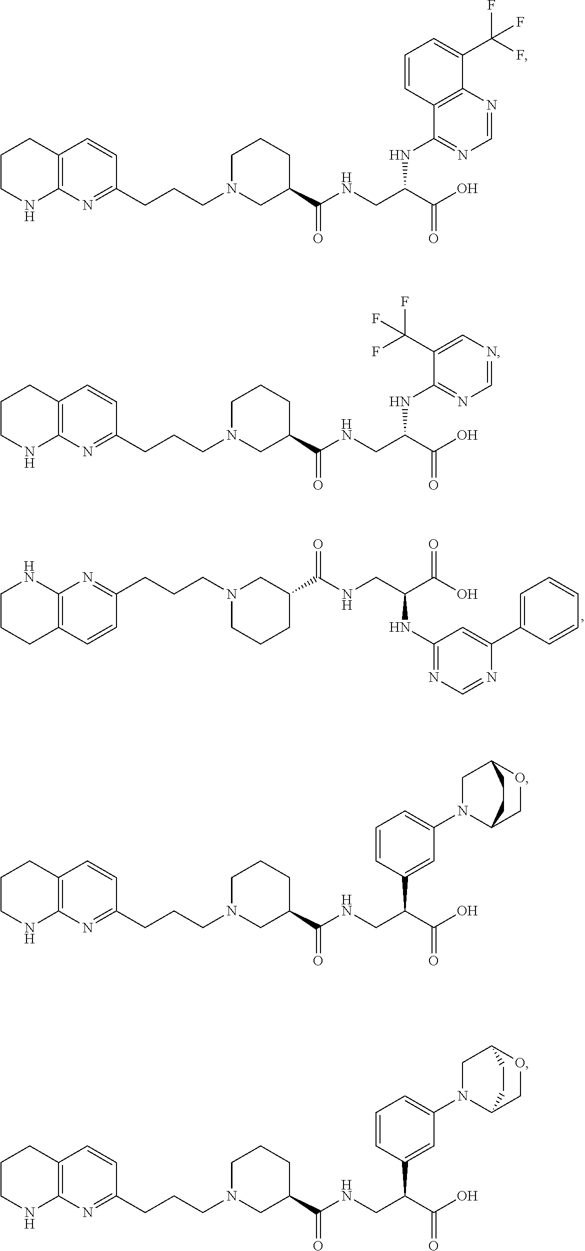 [SMILES: O=C(NC[C@H](Nc1ncnc2c(C(F)(F)F)cccc12)C(=O)O)[C@@H]1CCCN(CCCc2ccc3c(n2)NCCC3)C1 ; O=C(NC[C@H](Nc1ncncc1C(F)(F)F)C(=O)O)[C@@H]1CCCN(CCCc2ccc3c(n2)NCCC3)C1 ; O=C(NC[C@H](Nc1cc(-c2ccccc2)ncn1)C(=O)O)[C@@H]1CCCN(CCCc2ccc3c(n2)NCCC3)C1 ; O=C(NC[C@H](C(=O)O)c1cccc(N2C[C@H]3CC[C@@H]2CO3)c1)[C@@H]1CCCN(CCCc2ccc3c(n2)NCCC3)C1 ; O=C(NC[C@H](C(=O)O)c1cccc(N2C[C@@H]3CC[C@H]2CO3)c1)[C@@H]1CCCN(CCCc2ccc3c(n2)NCCC3)C1] 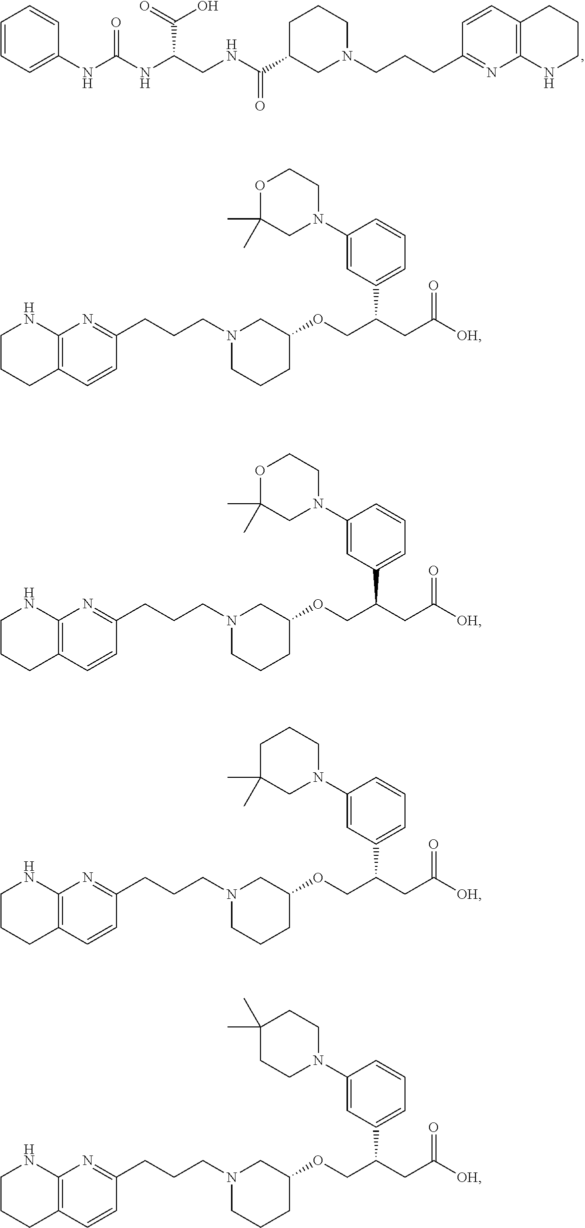 [SMILES: O=C(Nc1ccccc1)N[C@@H](CNC(=O)[C@@H]1CCCN(CCCc2ccc3c(n2)NCCC3)C1)C(=O)O ; CC1(C)CN(c2cccc([C@@H](CO[C@@H]3CCCN(CCCc4ccc5c(n4)NCCC5)C3)CC(=O)O)c2)CCO1 ; CC1(C)CN(c2cccc([C@H](CO[C@@H]3CCCN(CCCc4ccc5c(n4)NCCC5)C3)CC(=O)O)c2)CCO1 ; CC1(C)CCCN(c2cccc([C@@H](CO[C@@H]3CCCN(CCCc4ccc5c(n4)NCCC5)C3)CC(=O)O)c2)C1 ; CC1(C)CCN(c2cccc([C@@H](CO[C@@H]3CCCN(CCCc4ccc5c(n4)NCCC5)C3)CC(=O)O)c2)CC1] 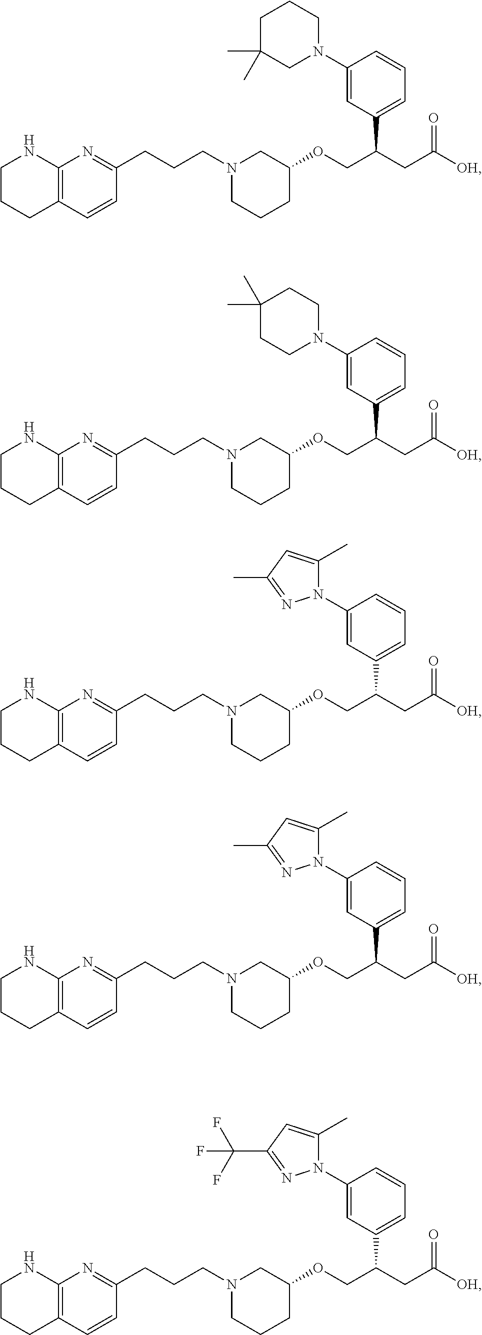 [SMILES: CC1(C)CCCN(c2cccc([C@H](CO[C@@H]3CCCN(CCCc4ccc5c(n4)NCCC5)C3)CC(=O)O)c2)C1 ; CC1(C)CCN(c2cccc([C@H](CO[C@@H]3CCCN(CCCc4ccc5c(n4)NCCC5)C3)CC(=O)O)c2)CC1 ; Cc1cc(C)n(-c2cccc([C@@H](CO[C@@H]3CCCN(CCCc4ccc5c(n4)NCCC5)C3)CC(=O)O)c2)n1 ; Cc1cc(C)n(-c2cccc([C@H](CO[C@@H]3CCCN(CCCc4ccc5c(n4)NCCC5)C3)CC(=O)O)c2)n1 ; Cc1cc(C(F)(F)F)nn1-c1cccc([C@@H](CO[C@@H]2CCCN(CCCc3ccc4c(n3)NCCC4)C2)CC(=O)O)c1] 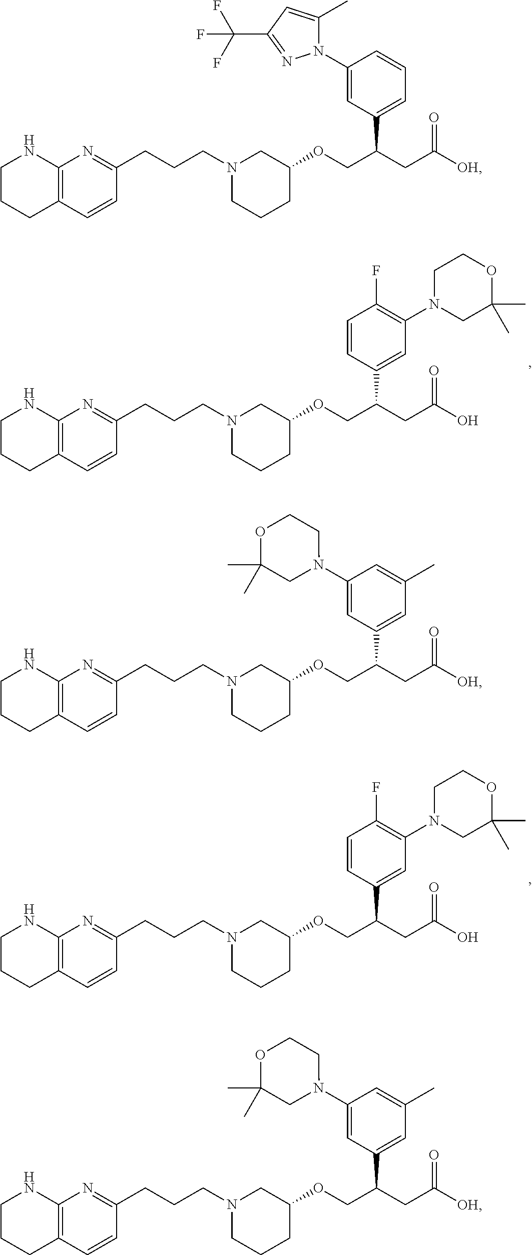 [SMILES: Cc1cc(C(F)(F)F)nn1-c1cccc([C@H](CO[C@@H]2CCCN(CCCc3ccc4c(n3)NCCC4)C2)CC(=O)O)c1 ; CC1(C)CN(c2cc([C@@H](CO[C@@H]3CCCN(CCCc4ccc5c(n4)NCCC5)C3)CC(=O)O)ccc2F)CCO1 ; Cc1cc([C@@H](CO[C@@H]2CCCN(CCCc3ccc4c(n3)NCCC4)C2)CC(=O)O)cc(N2CCOC(C)(C)C2)c1 ; CC1(C)CN(c2cc([C@H](CO[C@@H]3CCCN(CCCc4ccc5c(n4)NCCC5)C3)CC(=O)O)ccc2F)CCO1 ; Cc1cc([C@H](CO[C@@H]2CCCN(CCCc3ccc4c(n3)NCCC4)C2)CC(=O)O)cc(N2CCOC(C)(C)C2)c1] 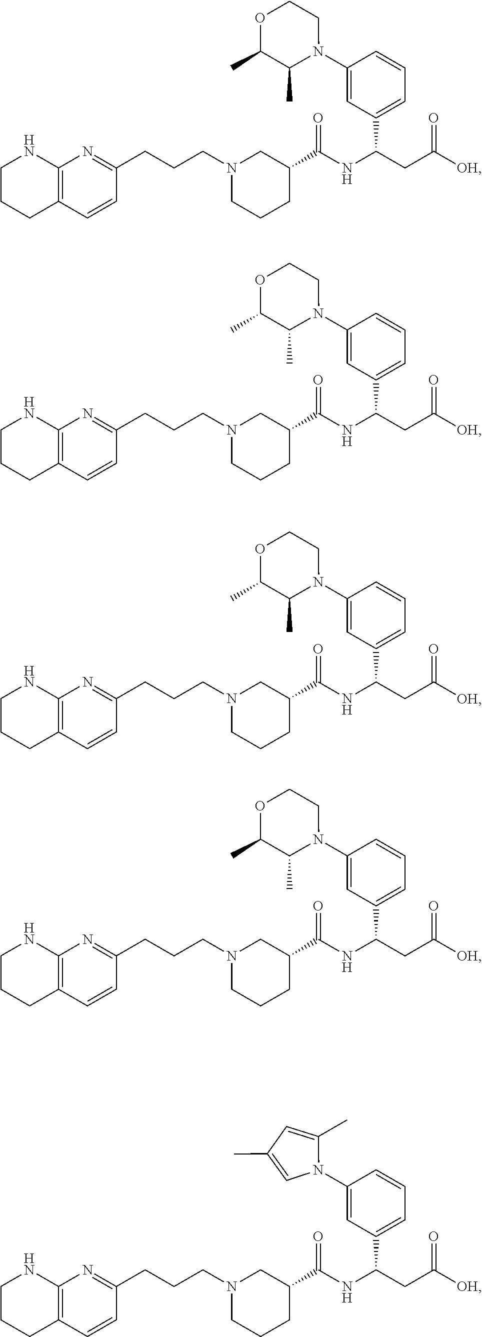 [SMILES: C[C@H]1OCCN(c2cccc([C@H](CC(=O)O)NC(=O)[C@@H]3CCCN(CCCc4ccc5c(n4)NCCC5)C3)c2)[C@H]1C ; C[C@@H]1OCCN(c2cccc([C@H](CC(=O)O)NC(=O)[C@@H]3CCCN(CCCc4ccc5c(n4)NCCC5)C3)c2)[C@@H]1C ; C[C@@H]1OCCN(c2cccc([C@H](CC(=O)O)NC(=O)[C@@H]3CCCN(CCCc4ccc5c(n4)NCCC5)C3)c2)[C@H]1C ; C[C@@H]1[C@@H](C)OCCN1c1cccc([C@H](CC(=O)O)NC(=O)[C@@H]2CCCN(CCCc3ccc4c(n3)NCCC4)C2)c1 ; Cc1cc(C)n(-c2cccc([C@H](CC(=O)O)NC(=O)[C@@H]3CCCN(CCCc4ccc5c(n4)NCCC5)C3)c2)c1] 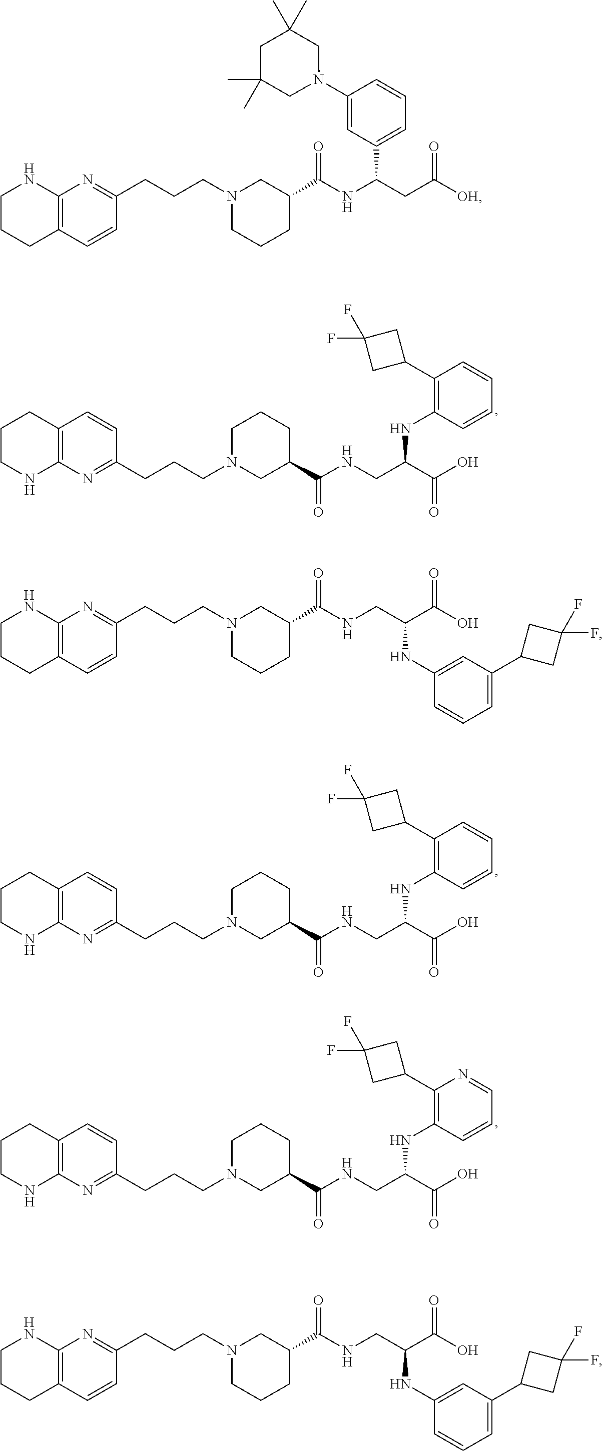 [SMILES: CC1(C)CN(c2cccc([C@H](CC(=O)O)NC(=O)[C@@H]3CCCN(CCCc4ccc5c(n4)NCCC5)C3)c2)CC(C)(C)C1 ; O=C(NC[C@@H](Nc1ccccc1C1CC(F)(F)C1)C(=O)O)[C@@H]1CCCN(CCCc2ccc3c(n2)NCCC3)C1 ; O=C(NC[C@@H](Nc1cccc(C2CC(F)(F)C2)c1)C(=O)O)[C@@H]1CCCN(CCCc2ccc3c(n2)NCCC3)C1 ; O=C(NC[C@H](Nc1ccccc1C1CC(F)(F)C1)C(=O)O)[C@@H]1CCCN(CCCc2ccc3c(n2)NCCC3)C1 ; O=C(NC[C@H](Nc1cccnc1C1CC(F)(F)C1)C(=O)O)[C@@H]1CCCN(CCCc2ccc3c(n2)NCCC3)C1 ; O=C(NC[C@H](Nc1cccc(C2CC(F)(F)C2)c1)C(=O)O)[C@@H]1CCCN(CCCc2ccc3c(n2)NCCC3)C1] 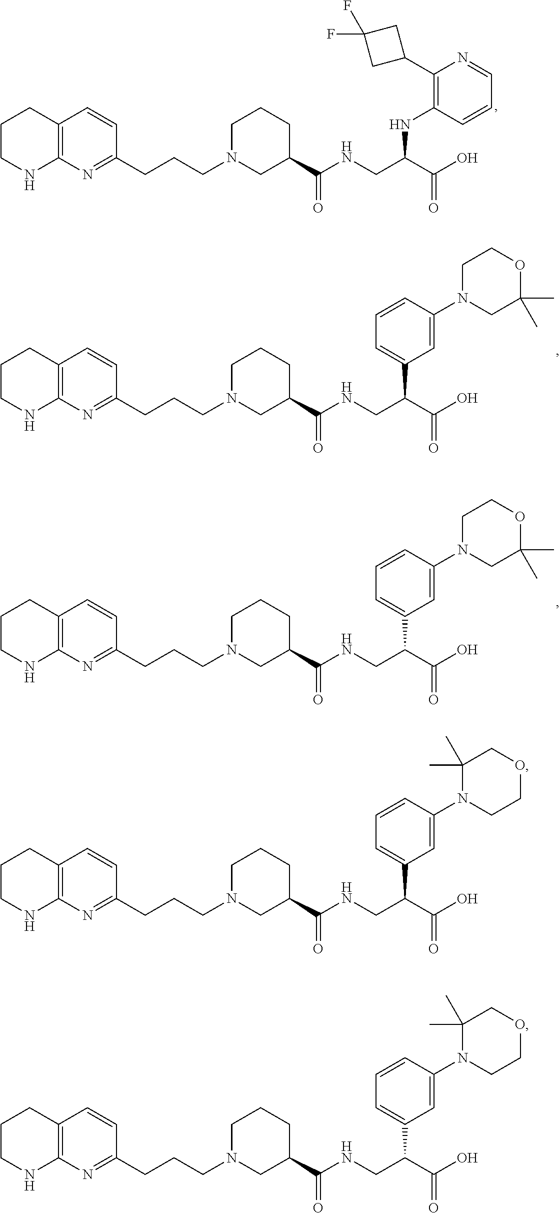 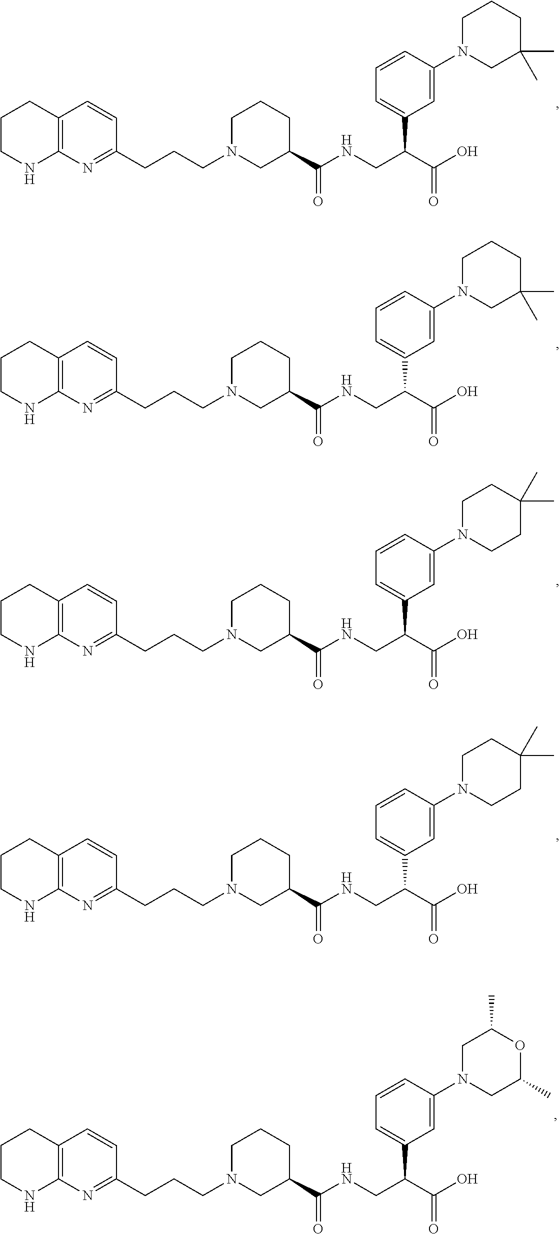 [SMILES: CC1(C)CCCN(c2cccc([C@H](CNC(=O)[C@@H]3CCCN(CCCc4ccc5c(n4)NCCC5)C3)C(=O)O)c2)C1 ; CC1(C)CCCN(c2cccc([C@@H](CNC(=O)[C@@H]3CCCN(CCCc4ccc5c(n4)NCCC5)C3)C(=O)O)c2)C1 ; CC1(C)CCN(c2cccc([C@H](CNC(=O)[C@@H]3CCCN(CCCc4ccc5c(n4)NCCC5)C3)C(=O)O)c2)CC1 ; CC1(C)CCN(c2cccc([C@@H](CNC(=O)[C@@H]3CCCN(CCCc4ccc5c(n4)NCCC5)C3)C(=O)O)c2)CC1 ; C[C@@H]1CN(c2cccc([C@H](CNC(=O)[C@@H]3CCCN(CCCc4ccc5c(n4)NCCC5)C3)C(=O)O)c2)C[C@H](C)O1] 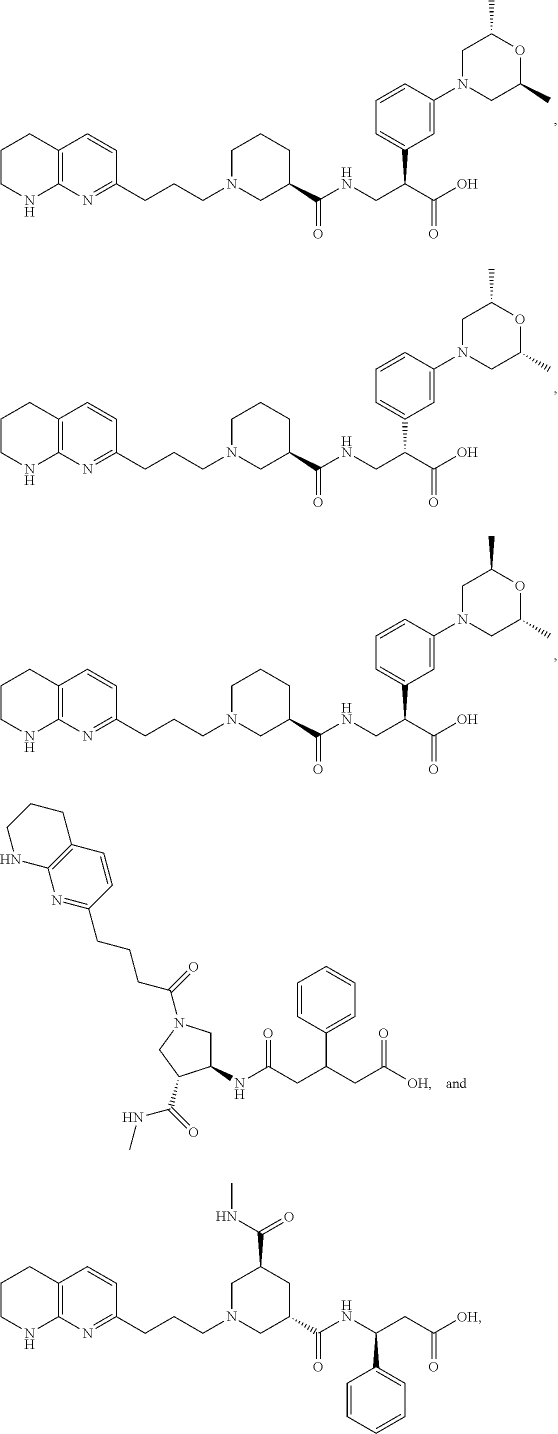 [SMILES: C[C@H]1CN(c2cccc([C@H](CNC(=O)[C@@H]3CCCN(CCCc4ccc5c(n4)NCCC5)C3)C(=O)O)c2)C[C@H](C)O1 ; C[C@@H]1CN(c2cccc([C@@H](CNC(=O)[C@@H]3CCCN(CCCc4ccc5c(n4)NCCC5)C3)C(=O)O)c2)C[C@H](C)O1 ; C[C@@H]1CN(c2cccc([C@H](CNC(=O)[C@@H]3CCCN(CCCc4ccc5c(n4)NCCC5)C3)C(=O)O)c2)C[C@@H](C)O1 ; CNC(=O)[C@@H]1CN(C(=O)CCCc2ccc3c(n2)NCCC3)C[C@H]1NC(=O)CC(CC(=O)O)c1ccccc1 ; CNC(=O)[C@H]1C[C@H](C(=O)N[C@@H](CC(=O)O)c2ccccc2)CN(CCCc2ccc3c(n2)NCCC3)C1]
or pharmaceutically acceptable salts thereof.

81. A pharmaceutical composition comprising pharmaceutically acceptable excipient and a compound or salt of any one of claim 1 to 80.

82. A method of modulating an alpha V integrin in a subject in need thereof, comprising administering to the subject a compound or salt of any one of claims 1 to 80 or a pharmaceutical composition of claim 81.

83. The method of claim 82, wherein the alpha V integrin is an alpha V beta 1 integrin.

84. The method of claim 82, wherein the alpha V integrin is an alpha V beta 6 integrin.

85. A method of treating a disease or condition comprising administering to a subject in need thereof a compound or salt of any one of claims 1 to 80 or a pharmaceutical composition of claim 81.

86. The method of claim 85, wherein the disease or condition is selected from: idiopathic pulmonary fibrosis, systemic lupus erythematosus associated interstitial lung disease, rheumatoid arthritis, diabetic nephropathy, focal segmental glomerulosclerosis, chronic kidney disease, nonalcoholic steatohepatitis, primary biliary cholangitis, primary sclerosing cholangitis, solid tumors, hematological tumors, organ transplant, Alport syndrome, interstitial lung disease, radiation-induced fibrosis, bleomycin-induced fibrosis, asbestos-induced fibrosis, flu-induced fibrosis, coagulation-induced fibrosis, vascular injury-induced fibrosis, aortic stenosis, and cardiac fibrosis.
